(19) United States
(12) Patent Application Publication
(10) Pub. No.: US 2025/0282798 A1
(43) Pub. Date: Sep. 11, 2025

(54) HETEROARYL-SUBSTITUTED BICYCLIC COMPOUND AND USE THEREOF

(71) Applicant: Medshine Discovery Inc., Nanjing, Jiangsu (CN)

(72) Inventors: Zhengxia Chen, Shanghai (CN); Yang Zhang, Shanghai (CN); Shuhui Chen, Shanghai (CN)

(73) Assignee: Medshine Discovery Inc., Nanjing, Jiangsu (CN)

(21) Appl. No.: 18/860,595

(22) PCT Filed: Apr. 27, 2023
(86) PCT No.: PCT/CN2023/091252

§ 371 Date: Oct. 25, 2024

(30) Foreign Application Priority Data
Apr. 27, 2022 (CN) 20221045171.1
Jun. 6, 2022 (CN) 202210632218.3
Jul. 11, 2022 (CN) 202210818491.5
Aug. 16, 2022 (CN) 202210983311.9
Sep. 26, 2022 (CN) 202211177697.0
Nov. 4, 2022 (CN) 202211429833.0
Dec. 15, 2022 (CN) 202211618983.6

(51) Int. Cl. C07D 519/00; A61K 31/437; A61K 31/4985; A61K 31/5383; A61K 31/55; A61K 31/551

(52) U.S. Cl. CPC C07D 519/00; A61K 31/437; A61K 31/4985; A61K 31/5383; A61K 31/55; A61K 31/551

(57) ABSTRACT

Disclosed are a series of heteroaryl-substituted bicyclic compounds and use thereof, and particularly, disclosed are a compound represented by formula (P), a stereoisomer thereof, and a pharmaceutically acceptable salt thereof.

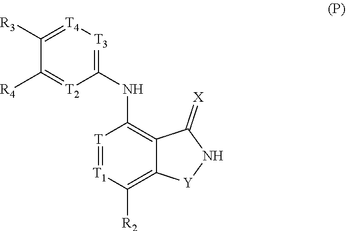

### CROSS REFERENCE TO RELATED APPLICATIONS

The present disclosure is a National Stage filing under 35 U.S.C. 371 of International PCT Application No. PCT/CN2023/091252, filed on Apr. 27, 2023, which claims the priority of:
CN202210457171.1, filed on Apr. 27, 2022;
CN202210632218.3, filed on Jun. 6, 2022;
CN202210818491.5, filed on Jul. 11, 2022;
CN202210983311.9, filed on Aug. 16, 2022;
CN202211177697.0, filed on Sep. 26, 2022;
CN202211429833.0, filed on Nov. 4, 2022; and
CN202211618983.6, filed on Dec. 15, 2022. The Chinese Patent Application Nos.
CN202210457171.1, filed on Apr. 27, 2022;
CN202210632218.3, filed on Jun. 6, 2022;
CN202210818491.5, filed on Jul. 11, 2022;
CN202210983311.9, filed on Aug. 16, 2022;
CN202211177697.0, filed on Sep. 26, 2022;
CN202211429833.0, filed on Nov. 4, 2022; and
CN202211618983.6, filed on Dec. 15, 2022 are incorporated herein by reference as part of the disclosure of the present application.

### TECHNICAL FIELD

The present disclosure relates to a series of heteroaryl-substituted bicyclic compounds and use thereof, and specifically relates to a compound represented by formula (P), stereoisomers and pharmaceutically acceptable salts thereof.

### BACKGROUND

Hematopoietic progenitor kinase 1 (HPK1) is an immunosuppressive regulatory kinase and a member of serine/threonine protein kinase superfamily (Serine/threonine kinases, SLK) of mammalian cells. It is a microtubule-associated protein, and restrictedly expressed in hematopoietic stem cells. HPK1 is a negative signal regulator of the T cell receptor (TCR). After TCR activation, cytoplasmic HPK1 is recruited to the vicinity of the cell membrane. The activated HPK1 phosphorylates the adaptor protein SLP76, thereby activating SLP76 as a docking site for the negative regulatory protein 14-3-3π, ultimately leading to the instability of the TCR signaling complex, thereby downregulating the TCR signal.

HPK1 can inhibit the proliferation, infiltration and metastasis of lung cancer cells. HPK1 deficiency also plays an important role in the pathogenesis of pancreatic ductal carcinoma, indicating that HPK1 kinase plays an extremely important role in disease treatment. Therefore, the discovery of small molecule inhibitors of HPK1 kinase is an urgent need for current drug research and development. We aim to develop highly active HPK1 inhibitors for tumor treatment.

### SUMMARY

The present disclosure provides a compound represented by formula (P), a stereoisomer or a pharmaceutically acceptable salt thereof,

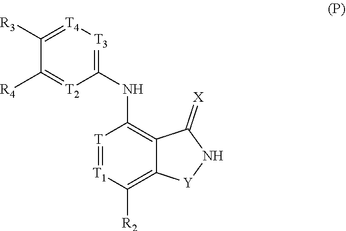

wherein,
X is selected from O and S;
Y is selected from C(R₈)₂ and C(R₈)═C(R₈);
T is selected from CH and N;
T₁ is selected from CH and N;
T₂ and T₃ are each independently selected from CH, CF and N;
T₄ is selected from CR₆ and N;
R₂ is heteroaryl having 5-membered ring fused to 6-membered ring, wherein the heteroaryl having 5-membered ring fused to 6-membered ring is optionally substituted with 1, 2 or 3 Rₐ;
R₃ and R₄ are taken together with the carbon atoms to which they are attached to form

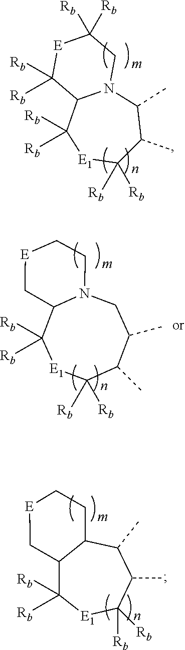

E and E₁ are each independently selected from —C(R₇)₂—, —O— and —N(R₅)—;
n and m are each independently selected from 0 and 1;
R₅ is selected from H and C₁₋₃ alkyl, wherein the C₁₋₃ alkyl is optionally substituted with 1, 2 or 3 R;
R₆ is selected from H, halogen and —C₁₋₃ alkyl-C₁₋₃ alkylamino;
R₇ is selected from H, F, Cl, Br, I, OH, NH₂, C₁₋₃ alkyl and C₁₋₃ alkoxy;
R₈ is selected from H and D;
each Rₐ is independently selected from D, halogen, C₁₋₃ alkyl and C₁₋₃ alkoxy, wherein the C₁₋₃ alkyl and C₁₋₃ alkoxy are each independently and optionally substituted with 1, 2 or 3 R;
each R_{b} is independently selected from H, D, OH, halogen and C₁₋₃ alkyl, wherein the C₁₋₃ alkyl is optionally substituted with 1, 2 or 3 halogens;
or, two R_{b} are taken together with the carbon atom to which they are jointly attached to form C═O or cyclopropyl;
each R is independently selected from halogen and D.

In some embodiments of the present disclosure, disclosed herein is the above compound, a stereoisomer or a pharmaceutically acceptable salt thereof, which is selected from:

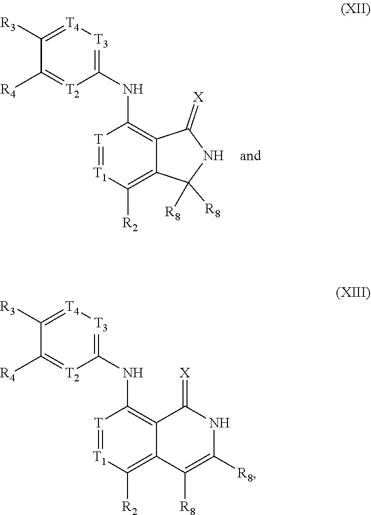

wherein,
X, T, T₁, T₂, T₃, T₄, R₂, R₃, R₄ and R₈ are as defined in the present disclosure.

In some embodiments of the present disclosure, each of the above Rₐ is independently selected from D, F, CH₃ and CD₃, and other variables are as defined in the present disclosure.

In some embodiments of the present disclosure, each of the above Rₐ is independently selected from D, F and CH₃, and other variables are as defined in the present disclosure.

In some embodiments of the present disclosure, each of the above R_{b} is independently selected from H, D, F, Cl, Br, I, OH, CH₃, CH₂CH₃ and CH₂CH₂CH₃, wherein the CH₃, CH₂CH₃ and CH₂CH₂CH₃ are optionally substituted with 1, 2 or 3 halogens, and other variables are as defined in the present disclosure.

In some embodiments of the present disclosure, each of the above R_{b} is independently selected from H, D, CH₃, CH₂CH₃ and CH₂CH₂CH₃, wherein the CH₃, CH₂CH₃ and CH₂CH₂CH₃ are optionally substituted with 1, 2 or 3 halogens, and other variables are as defined in the present disclosure.

In some embodiments of the present disclosure, each of the above R_{b} is independently selected from H, D, F, OH, CH₃ and CF₃, and other variables are as defined in the present disclosure.

In some embodiments of the present disclosure, each of the above R_{b} is independently selected from H, D, CH₃ and CF₃, and other variables are as defined in the present disclosure.

In some embodiments of the present disclosure, the above R₂ is selected from pyrrolopyridinyl and imidazopyridinyl, wherein the pyrrolopyridinyl and imidazopyridinyl are optionally substituted with 1, 2 or 3 Rₐ, and Rₐ and other variables are as defined in the present disclosure.

In some embodiments of the present disclosure, the above R₂ is selected from

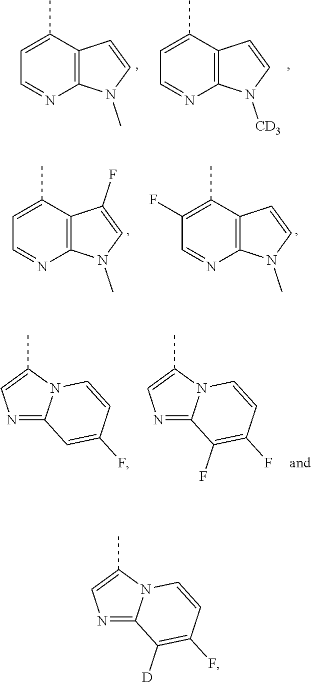

and other variables are as defined in the present disclosure.

In some embodiments of the present disclosure, the above R₂ is selected from

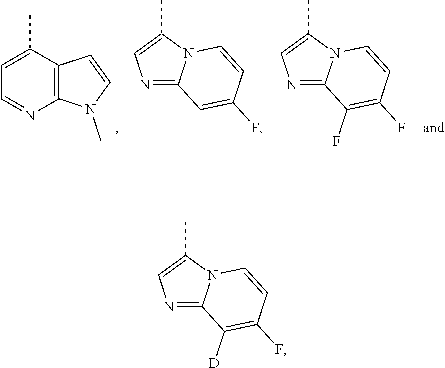

and other variables are as defined in the present disclosure.

In some embodiments of the present disclosure, the above R₆ is selected from H, F and —CH₂—N(CH₃)₂, and other variables are as defined in the present disclosure.

In some embodiments of the present disclosure, the above R₆ is selected from H and F, and other variables are as defined in the present disclosure.

In some embodiments of the present disclosure, the above R₅ is selected from H, CH₃, CD₃, CH₂CH₃ and CH(CH₃)₂, and other variables are as defined in the present disclosure.

In some embodiments of the present disclosure, the above R₇ is selected from H, F, Cl, Br, I, OH, NH₂, CH₃, CH₂CH₃ and CH₂CH₂CH₃, and other variables are as defined in the present disclosure.

In some embodiments of the present disclosure, the above R₇ is selected from H, F, OH, CH₃ and OCH₃, and other variables are as defined in the present disclosure.

In some embodiments of the present disclosure, the above R₇ is selected from F, OH, CH₃ and OCH₃, and other variables are as defined in the present disclosure.

In some embodiments of the present disclosure, the above R₇ is selected from F, OH and CH₃, and other variables are as defined in the present disclosure.

In some embodiments of the present disclosure, the above T₂ is selected from N, CF and CH, and other variables are as defined in the present disclosure.

In some embodiments of the present disclosure, the above T₃ is selected from CH and N, and other variables are as defined in the present disclosure.

In some embodiments of the present disclosure, the above T₄ is selected from CH and N, and other variables are as defined in the present disclosure.

In some embodiments of the present disclosure, the above E and E₁ are each independently selected from —CH₂—, —CHF—, —CF₂—, —CH(OH)—, —CH(CH₃)—,

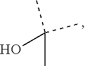

—O—, —NH—, —N(CH₃)—, —N(CD₃)-, —N(CH₂CH₃)—,

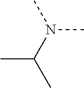

and —CH(OCH₃)—, and other variables are as defined in the present disclosure.

In some embodiments of the present disclosure, the above E and E₁ are each independently selected from —CH₂—, —CHF—, —CF₂—, —CH(OH)—, —CH(CH₃)—,

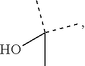

—O—, —NH—, —N(CH₃)—, —N(CD₃)-, —N(CH₂CH₃)— and

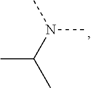

and other variables are as defined in the present disclosure.

In some embodiments of the present disclosure, the above E is selected from —CH₂—, —CHF—, —CF₂—, —CH(OH)—, —CH(CH₃)—,

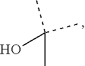

—O—, —NH—, —N(CH₃)—, —N(CD₃)-, —N(CH₂CH₃)— and

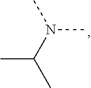

and other variables are as defined in the present disclosure.

In some embodiments of the present disclosure, the above E₁ is selected from —CH₂—, —CHF—, —CF₂—, —CH(OH)—, —CH(CH₃)—,

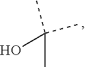

—O—, —NH—, —N(CH₃)—, —N(CD₃)-, —N(CH₂CH₃)— and

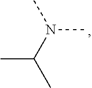

and other variables are as defined in the present disclosure.

In some embodiments of the present disclosure, the above E and E₁ are each independently selected from —CH₂—, —CF₂—, —CH(OH)—, —CH(CH₃)—, —O—, —NH—, —N(CH₃)—, —N(CH₂CH₃)— and

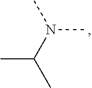

and other variables are as defined in the present disclosure.

In some embodiments of the present disclosure, the above E and E₁ are each independently selected from —CH₂—, —C(OH)—, —C(CH₃)—, —O—, NH, —N(CH₃)— and —N(CH₂CH₃)—, and other variables are as defined in the present disclosure.

In some embodiments of the present disclosure, the above R₃ and R₄ are taken together with the carbon atoms to which they are attached to form

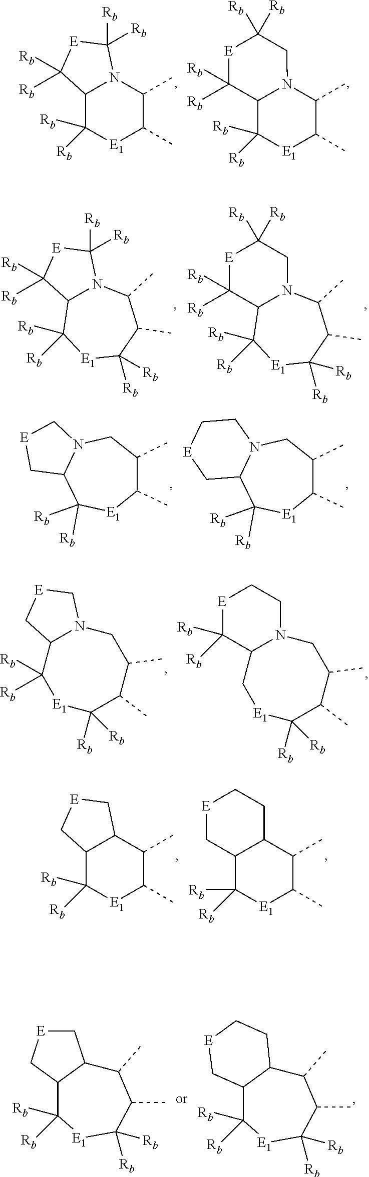

and other variables are as defined in the present disclosure.

In some embodiments of the present disclosure, the above R₃ and R₄ are taken together with the carbon atoms to which they are attached to form

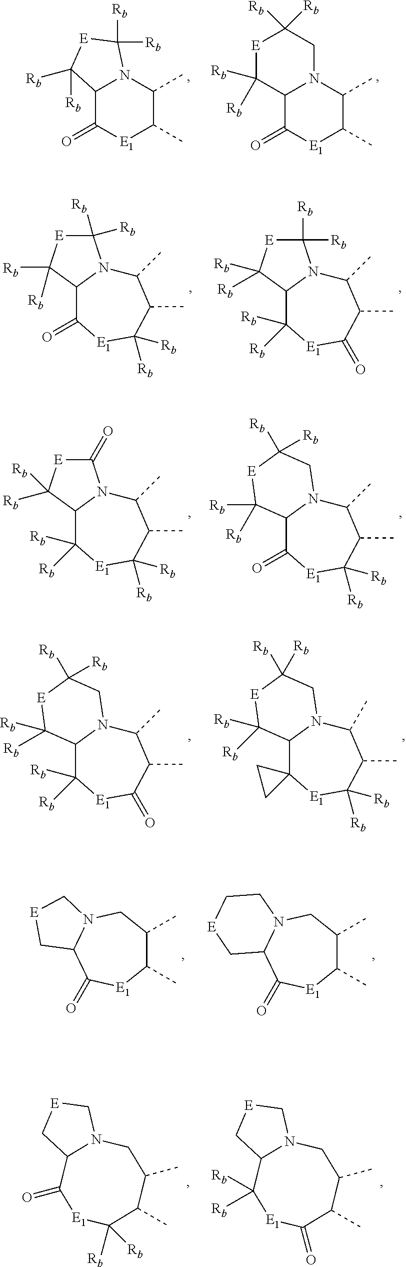

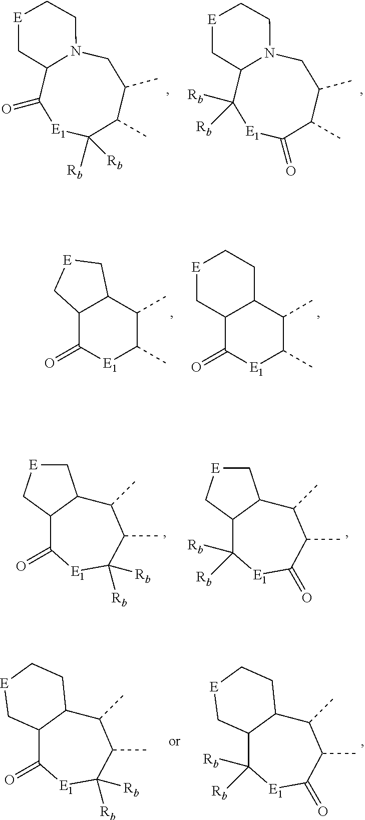

and each R_{b} and other variables are as defined in the present disclosure.

In some embodiments of the present disclosure, the above R₃ and R₄ are taken together with the carbon atoms to which they are attached to form

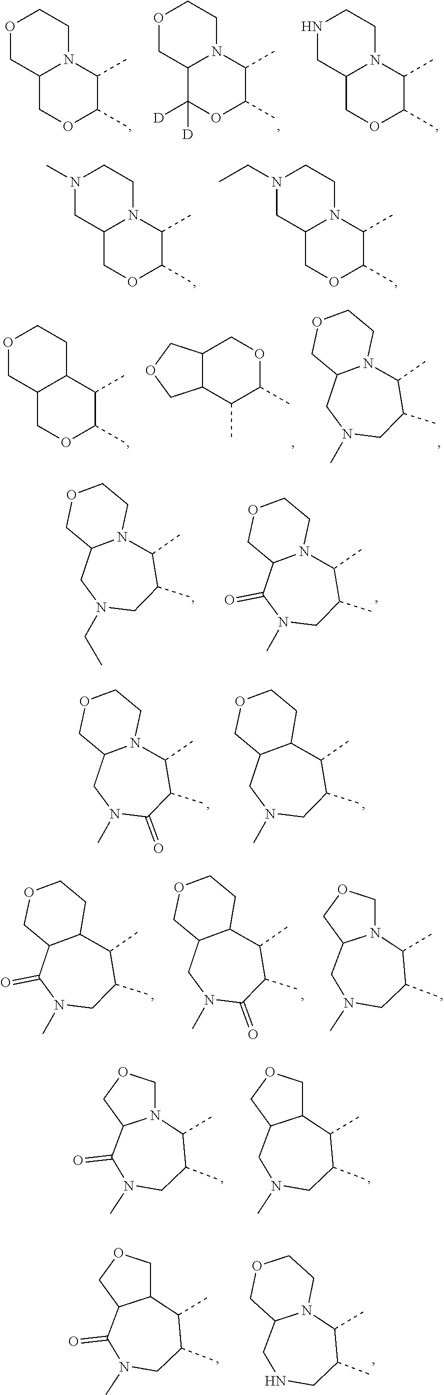

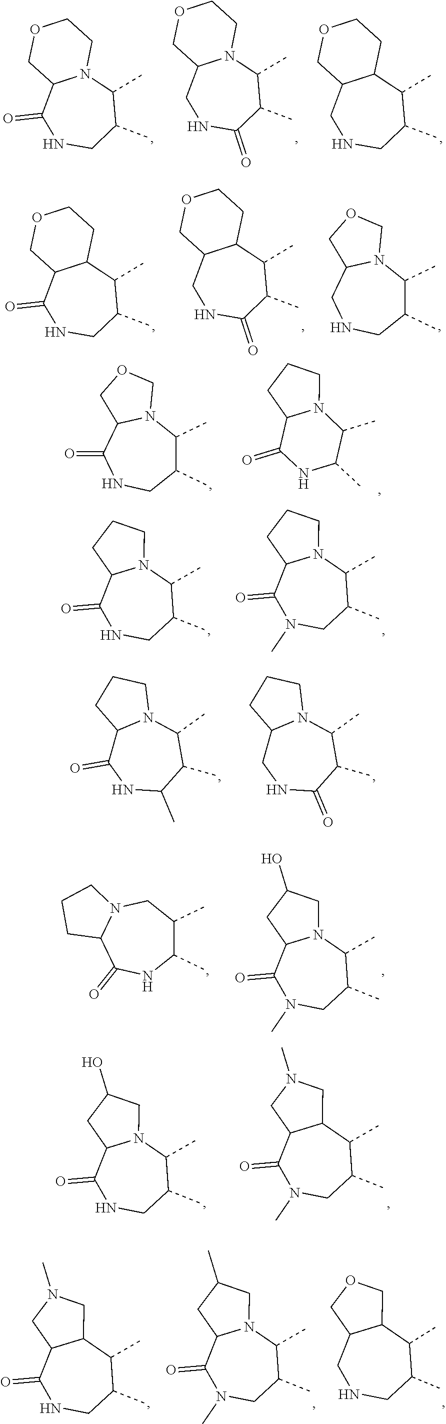

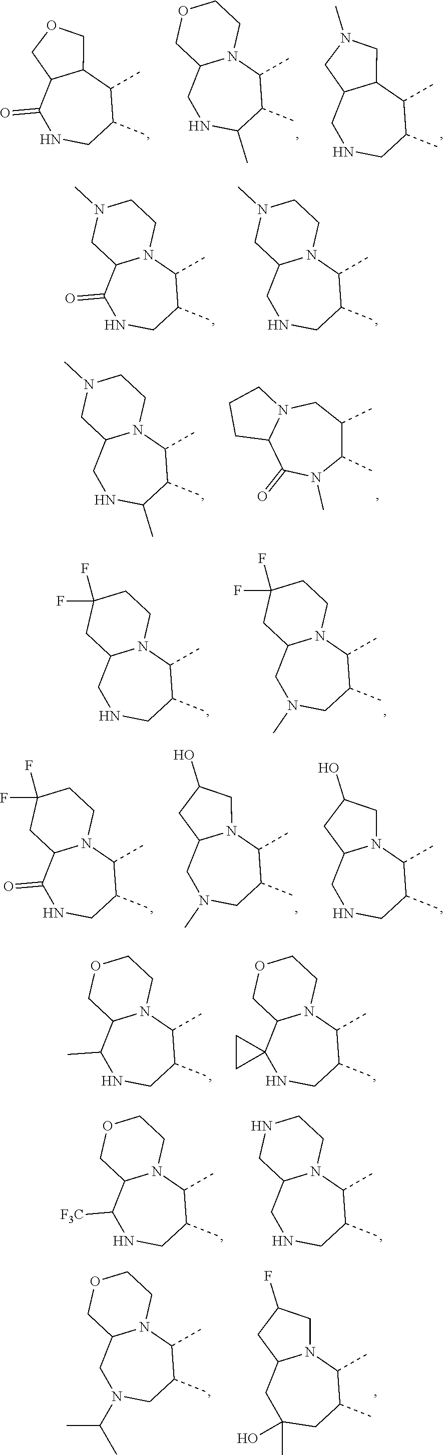

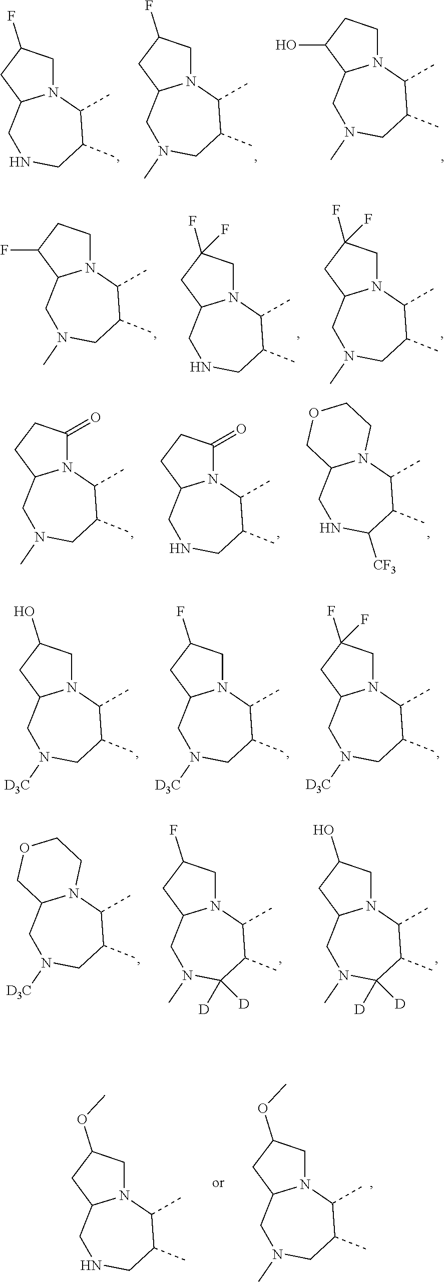

and other variables are as defined in the present disclosure.

The present disclosure also provides a compound represented by formula (XII), a stereoisomer or a pharmaceutically acceptable salt thereof,

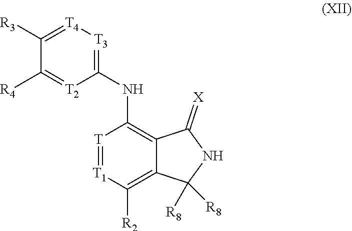

wherein,
X is selected from O and S;
T₁ is selected from CH and N;
T₂ and T₃ are each independently selected from CH, CF and N;
T₄ is selected from CR₆ and N;
R₂ is heteroaryl having 5-membered ring fused to 6-membered ring, wherein the heteroaryl having 5-membered ring fused to 6-membered ring is optionally substituted with 1, 2 or 3 Rₐ;
when T is CH or N, R₃ and R₄ are taken together with the carbon atoms to which they are attached to form

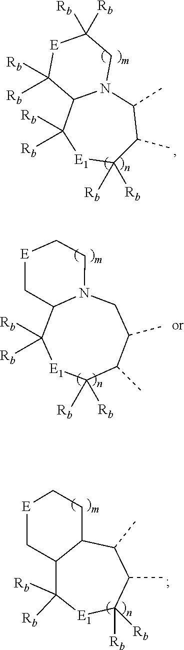

or, when T is CF or N, R₃ is

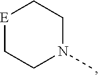

and R₄ is selected from C₁₋₃ alkyl, C₁₋₃ alkoxy, C₁₋₃ alkylamino and —CH₂—C₁₋₃ alkylamino;
E and E₁ are each independently selected from —C(R₇)₂—, —O— and —N(R₅)—;
n and m are each independently selected from 0 and 1;
R₅ is selected from H and C₁₋₃ alkyl, wherein the C₁₋₃ alkyl is optionally substituted with 1, 2 or 3 R;
R₆ is selected from H, halogen and —C₁₋₃ alkyl-C₁₋₃ alkylamino;
R₇ is selected from H, F, Cl, Br, I, OH, NH₂, C₁₋₃ alkyl and C₁₋₃ alkoxy;
R₈ is selected from H and D;
each Rₐ is independently selected from D, halogen, C₁₋₃ alkyl and C₁₋₃ alkoxy, wherein the C₁₋₃ alkyl and C₁₋₃ alkoxy are each independently and optionally substituted with 1, 2 or 3 R;
each R_{b} is independently selected from H, D, OH, halogen and C₁₋₃ alkyl, wherein the C₁₋₃ alkyl is optionally substituted with 1, 2 or 3 halogens;
or, two R_{b} are taken together with the carbon atom to which they are jointly attached to form C═O or cyclopropyl;
each R is independently selected from halogen and D.

The present disclosure also provides a compound represented by formula (XIII), a stereoisomer or a pharmaceutically acceptable salt thereof,

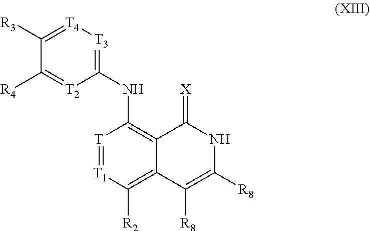

wherein,
X is selected from O and S;
T₁ is selected from CH and N;
T₂ and T₃ are each independently selected from CH, CF and N;
T₄ is selected from CR₆ and N;
R₂ is heteroaryl having 5-membered ring fused to 6-membered ring, wherein the heteroaryl having 5-membered ring fused to 6-membered ring is optionally substituted with 1, 2 or 3 Rₐ;
when T is CH or N, R₃ and R₄ are taken together with the carbon atoms to which they are attached to form

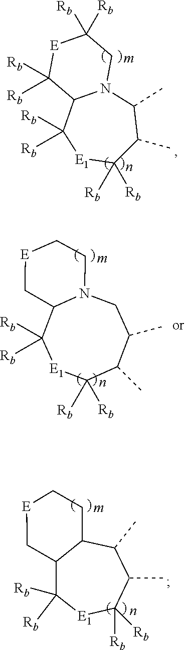

or, when T is CF or N, R₃ is

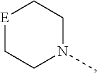

and R₄ is selected from C₁₋₃ alkyl, C₁₋₃ alkoxy, C₁₋₃ alkylamino and —CH₂—C₁₋₃ alkylamino;
E and E₁ are each independently selected from —C(R₇)₂—, —O— and —N(R₅)—;
n and m are each independently selected from 0 and 1;
R₅ is selected from H and C₁₋₃ alkyl, wherein the C₁₋₃ alkyl is optionally substituted with 1, 2 or 3 R;
R₆ is selected from H, halogen and —C₁₋₃ alkyl-C₁₋₃ alkylamino;
R₇ is selected from H, F, Cl, Br, I, OH, NH₂, C₁₋₃ alkyl and C₁₋₃ alkoxy;
R₈ is selected from H and D;
each Rₐ is independently selected from D, halogen, C₁₋₃ alkyl and C₁₋₃ alkoxy, wherein the C₁₋₃ alkyl and C₁₋₃ alkoxy are each independently and optionally substituted with 1, 2 or 3 R;
each R_{b} is independently selected from H, D, OH, halogen and C₁₋₃ alkyl, wherein the C₁₋₃ alkyl is optionally substituted with 1, 2 or 3 halogens;
or, two R_{b} are taken together with the carbon atom to which they are jointly attached to form C═O or cyclopropyl;
each R is independently selected from halogen and D.

The present disclosure also provides a compound represented by formula (XII), a stereoisomer or a pharmaceutically acceptable salt thereof,

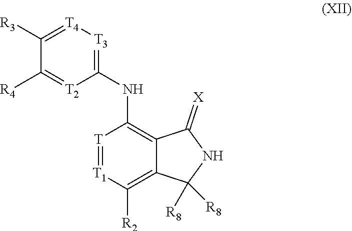

wherein,
X is selected from O and S;
T is selected from CH, CF and N;
T₁ is selected from CH and N;
T₂ and T₃ are each independently selected from CH, CF and N;
   T₄ is selected from CR₆ and N;
   R₂ is heteroaryl having 5-membered ring fused to 6-membered ring, wherein the heteroaryl having 5-membered ring fused to 6-membered ring is optionally substituted with 1, 2 or 3 Rₐ;
   when T is CH or N, R₃ and R₄ are taken together with the carbon atoms to which they are attached to form

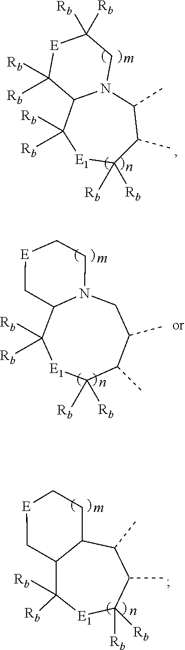

or, when T is CF or N, R₃ is

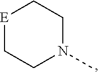

and R₄ is selected from C₁₋₃ alkyl, C₁₋₃ alkoxy, C₁₋₃ alkylamino and —CH₂—C₁₋₃ alkylamino;
E and E₁ are each independently selected from —C(R₇)₂—, —O— and —N(R₅)—;
n and m are each independently selected from 0 and 1;
R₅ is selected from H and C₁₋₃ alkyl, wherein the C₁₋₃ alkyl is optionally substituted with 1, 2 or 3 R;
R₆ is selected from H, halogen and —C₁₋₃ alkyl-C₁₋₃ alkylamino;
R₇ is selected from H, F, Cl, Br, I, OH, NH₂ and C₁₋₃ alkyl;
R₈ is selected from H and D;
each Rₐ is independently selected from D, halogen, C₁₋₃ alkyl and C₁₋₃ alkoxy, wherein the C₁₋₃ alkyl and C₁₋₃ alkoxy are each independently and optionally substituted with 1, 2 or 3 R;
each R_{b} is independently selected from H, D, OH, halogen and C₁₋₃ alkyl, wherein the C₁₋₃ alkyl is optionally substituted with 1, 2 or 3 halogens;
or, two R_{b} are taken together with the carbon atom to which they are jointly attached to form C═O or cyclopropyl;
R is selected from halogen and D.

The present disclosure also provides a compound represented by formula (V), a stereoisomer or a pharmaceutically acceptable salt thereof,

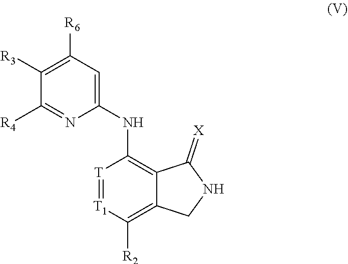

wherein,
X is selected from O and S;
T is selected from CH, CF and N;
T₁ is selected from CH and N;
R₂ is heteroaryl having 5-membered ring fused to 6-membered ring, wherein the heteroaryl having 5-membered ring fused to 6-membered ring is optionally substituted with 1, 2 or 3 Rₐ;
when T is CH or N, R₃ and R₄ are taken together with the carbon atoms to which they are attached to form

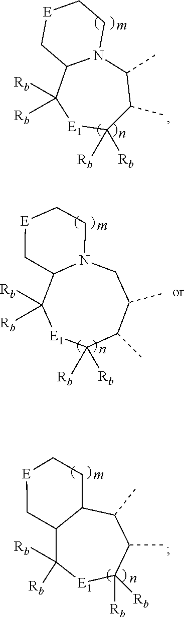

or, when T is CF or N, R₃ is

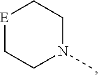

and R₄ is selected from C₁₋₃ alkyl, C₁₋₃ alkoxy, C₁₋₃ alkylamino and —CH₂—C₁₋₃ alkylamino;
E and E₁ are each independently selected from —C(R₇)₂—, —O— and —N(R₅)—;
n and m are each independently selected from 0 and 1;
R₅ is selected from H and C₁₋₃ alkyl;
R₆ is selected from H and —C₁₋₃ alkyl-C₁₋₃ alkylamino;
R₇ is selected from H, F, Cl, Br, I, OH, NH₂ and C₁₋₃ alkyl;
each Rₐ is independently selected from D, halogen, C₁₋₃ alkyl and C₁₋₃ alkoxy;
each R_{b} is independently selected from H, D and C₁₋₃ alkyl, wherein the C₁₋₃ alkyl is optionally substituted with 1, 2 or 3 halogens;
or, two R_{b} are taken together with the carbon atom to which they are jointly attached to form C═O or cyclopropyl;
The present disclosure also provides a compound represented by formula (V), a stereoisomer or a pharmaceutically acceptable salt thereof,

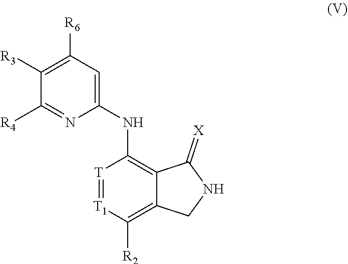

wherein,
X is selected from O and S;
T is selected from CH, CF and N;
T₁ is selected from CH and N;
R₂ is heteroaryl having 5-membered ring fused to 6-membered ring, wherein the heteroaryl having 5-membered ring fused to 6-membered ring is optionally substituted with 1, 2 or 3 Rₐ;
when T is CH or N, R₃ and R₄ are taken together with the carbon atoms to which they are attached to form

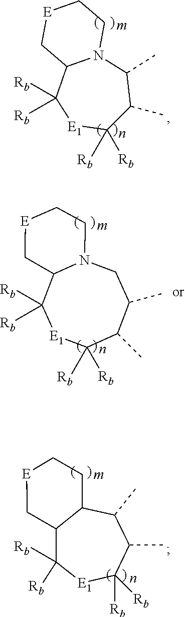

or, when T is CF or N, R₃ is

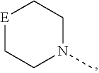

and R₄ is selected from C₁₋₃ alkyl, C₁₋₃ alkoxy, C₁₋₃ alkylamino and —CH₂—C₁₋₃ alkylamino;
E and E₁ are each independently selected from —C(R₇)₂—, —O— and —N(R₅)—;
n and m are each independently selected from 0 and 1;
R₅ is selected from H and C₁₋₃ alkyl;
R₆ is selected from H and —C₁₋₃ alkyl-C₁₋₃ alkylamino;
R₇ is selected from H, F, Cl, Br, I, OH, NH₂ and C₁₋₃ alkyl;

each Rₐ is independently selected from D, halogen, C₁₋₃ alkyl and C₁₋₃ alkoxy;

each R_{b} is independently selected from H, D and C₁₋₃ alkyl;

or, two R_{b} are taken together with the carbon atom to which they are jointly attached to form C═O.

The present disclosure also provides a compound represented by formula (V), a stereoisomer or a pharmaceutically acceptable salt thereof,

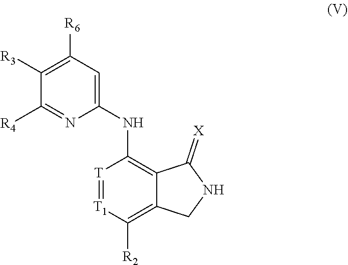

wherein,
X is selected from O and S;
T is selected from CH, CF and N;
T₁ is selected from CH and N;
R₂ is heteroaryl having 5-membered ring fused to 6-membered ring, wherein the heteroaryl having 5-membered ring fused to 6-membered ring is optionally substituted with 1, 2 or 3 Rₐ;
when T is CH or N, R₃ and R₄ are taken together with the carbon atoms to which they are attached to form

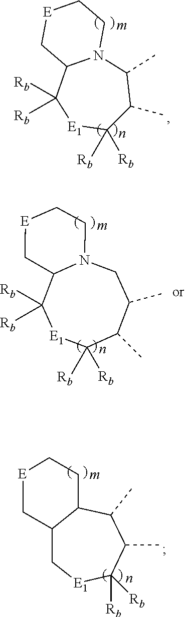

when T is CF or N, R₃ is

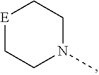

and R₄ is selected from C₁₋₃ alkyl, C₁₋₃ alkoxy, C₁₋₃ alkylamino and —CH₂—C₁₋₃ alkylamino;
E and E₁ are each independently selected from —C(R₇)₂—, —O— and —N(R₅)—;
n and m are each independently selected from 0 and 1;
R₅ is selected from H and C₁₋₃ alkyl;
R₆ is selected from H and —C₁₋₃ alkyl-C₁₋₃ alkylamino;
R₇ is selected from H, F, Cl, Br, I, OH, NH₂ and C₁₋₃ alkyl;
each Rₐ is independently selected from D, halogen, C₁₋₃ alkyl and C₁₋₃ alkoxy;
each R_{b} is independently selected from H, D and C₁₋₃ alkyl;
or, two R_{b} are taken together with the carbon atom to which they are jointly attached to form C═O.

The present disclosure also provides a compound represented by formula (V), a stereoisomer or a pharmaceutically acceptable salt thereof,

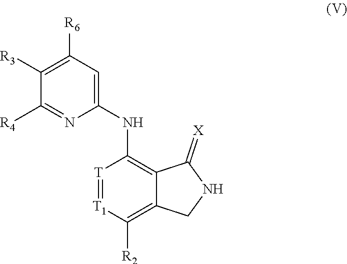

wherein,
X is selected from O and S;
T is selected from CH, CF and N;
T₁ is selected from CH and N;
R₂ is heteroaryl having 5-membered ring fused to 6-membered ring, wherein the heteroaryl having 5-membered ring fused to 6-membered ring is optionally substituted with 1, 2 or 3 Rₐ;
when T is CH or N, R₃ and R₄ are taken together with the carbon atoms to which they are attached to form

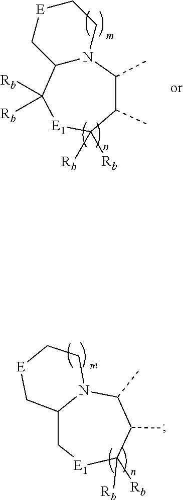

when T is CF or N, R₃ is

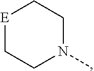

and R₄ is selected from C₁₋₃ alkyl, C₁₋₃ alkoxy, C₁₋₃ alkylamino and —CH₂—C₁₋₃ alkylamino;
E and E₁ are each independently selected from —O— and —N(R₅)—;
n and m are each independently selected from 0 and 1;
R₅ is selected from H and C₁₋₃ alkyl;
R₆ is selected from H and —C₁₋₃ alkyl-C₁₋₃ alkylamino;
each Rₐ is independently selected from D, halogen, C₁₋₃ alkyl and C₁₋₃ alkoxy;
each R_{b} is independently selected from H and D;
or, two R_{b} are taken together with the carbon atom to which they are jointly attached to form C═O.

In some embodiments of the present disclosure, each of the above Rₐ is independently selected from D, F, CH₃ and CD₃, and other variables are as defined in the present disclosure.

In some embodiments of the present disclosure, each of the above R_{b} is independently selected from H, D, F, OH, CH₃ and CF₃, and other variables are as defined in the present disclosure.

In some embodiments of the present disclosure, the above R₂ is selected from pyrrolopyridinyl and imidazopyridinyl, wherein the pyrrolopyridinyl and imidazopyridinyl are optionally substituted with 1, 2 or 3 Rₐ, and Rₐ and other variables are as defined in the present disclosure.

In some embodiments of the present disclosure, the above R₂ is selected from pyrrolopyridinyl and imidazopyridinyl, wherein the pyrrolopyridinyl and imidazopyridinyl are optionally substituted with 1, 2 or 3 Rₐ, and other variables are as defined in the present disclosure.

In some embodiments of the present disclosure, the above R₂ is selected from

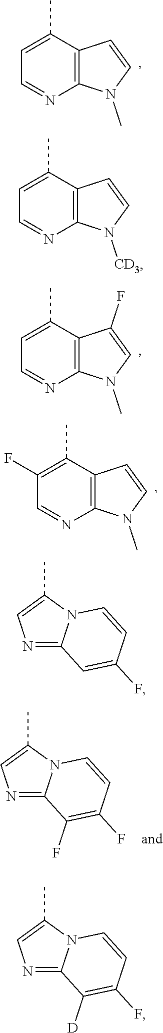

and other variables are as defined in the present disclosure.

In some embodiments of the present disclosure, the above R₆ is selected from H, F and —CH₂—N(CH₃)₂, and other variables are as defined in the present disclosure.

In some embodiments of the present disclosure, the above R₆ is selected from H and —CH₂—N(CH₃)₂, and other variables are as defined in the present disclosure.

In some embodiments of the present disclosure, the above R₅ is selected from H, CH₃, CD₃, CH₂CH₃ and CH(CH₃)₂, and other variables are as defined in the present disclosure.

In some embodiments of the present disclosure, the above R₇ is selected from H, F, OH, CH₃ and OCH₃, and other variables are as defined in the present disclosure.

In some embodiments of the present disclosure, the above T₂ is selected from N, CF and CH, and other variables are as defined in the present disclosure.

In some embodiments of the present disclosure, the above T₃ is selected from CH and N, and other variables are as defined in the present disclosure.

In some embodiments of the present disclosure, the above T₄ is selected from CH and N, and other variables are as defined in the present disclosure.

In some embodiments of the present disclosure, the above E and E₁ are each independently selected from —CH₂—, —CHF—, —CF₂—, —CH(OH)—, —CH(CH₃)—,

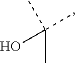

—O—, —NH—, —N(CH₃)—, —N(CD₃)-, —N(CH₂CH₃)—,

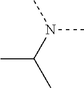

and —CH(OCH₃)—, and other variables are as defined in the present disclosure.

In some embodiments of the present disclosure, the above E and E₁ are each independently selected from —O—, —N(CH₃)— and —N(CH₂CH₃)—, and other variables are as defined in the present disclosure.

In some embodiments of the present disclosure, when the above T is CH or N, R₃ and R₄ are taken together with the carbon atoms to which they are attached to form

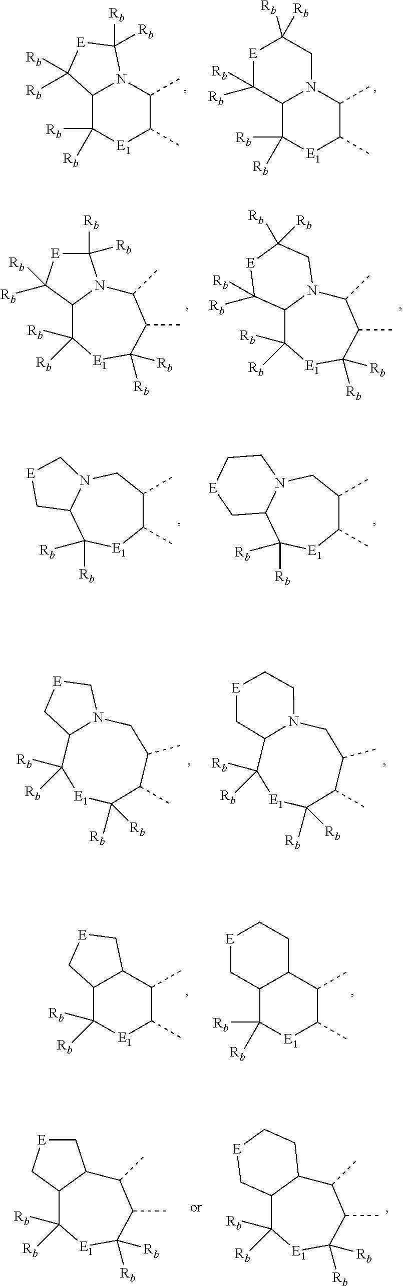

and other variables are as defined in the present disclosure.

In some embodiments of the present disclosure, when the above T is CH or N, R₃ and R₄ are taken together with the carbon atoms to which they are attached to form

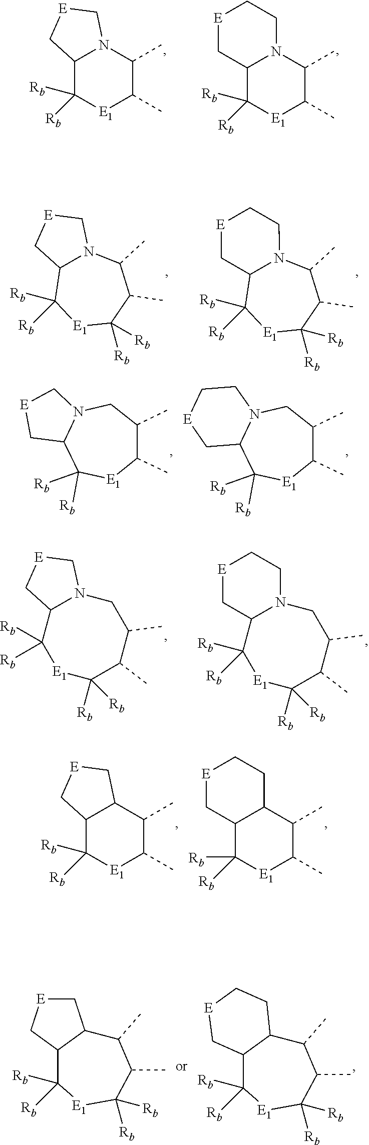

and other variables are as defined in the present disclosure.

In some embodiments of the present disclosure, when the above T is CH or N, R₃ and R₄ are taken together with the carbon atoms to which they are attached to form

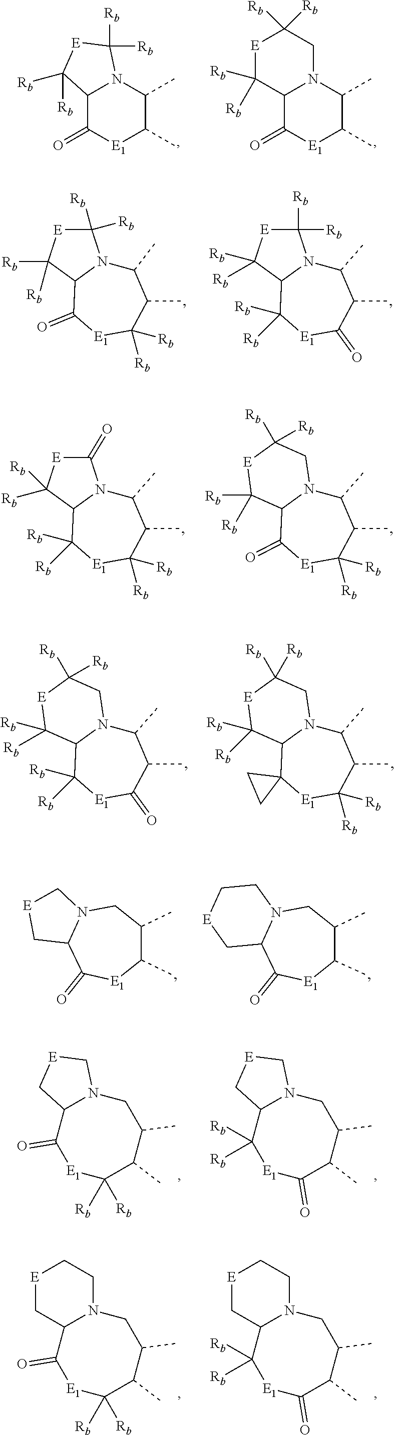

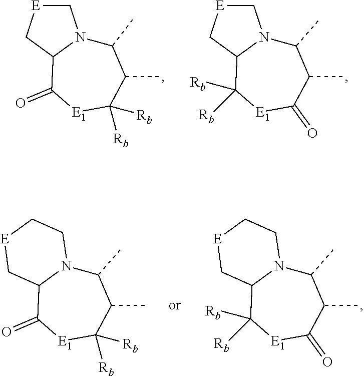

each R_{b} is independently selected from H, D, OH, halogen and C₁₋₃ alkyl, wherein the C₁₋₃ alkyl is optionally substituted with 1, 2 or 3 halogens, and other variables are as defined in the present disclosure.

In some embodiments of the present disclosure, the above R₃ and R₄ are taken together with the carbon atoms to which they are attached to form

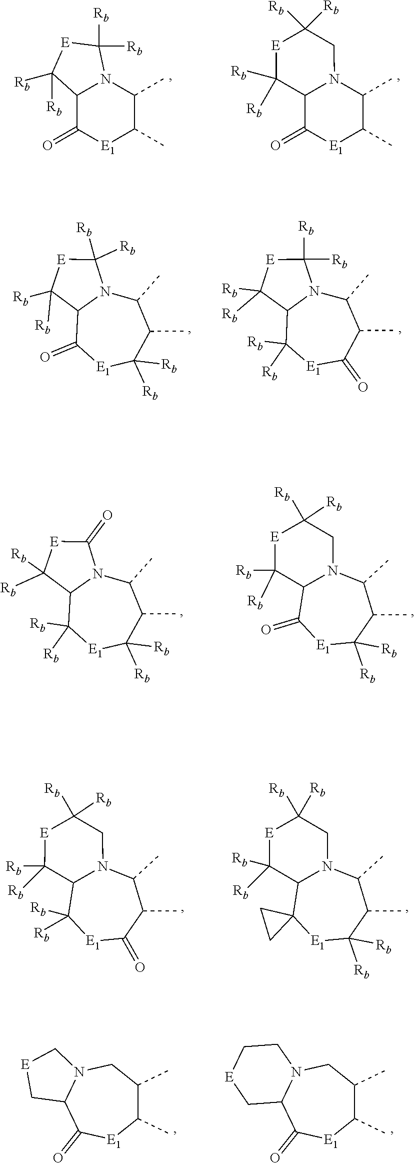

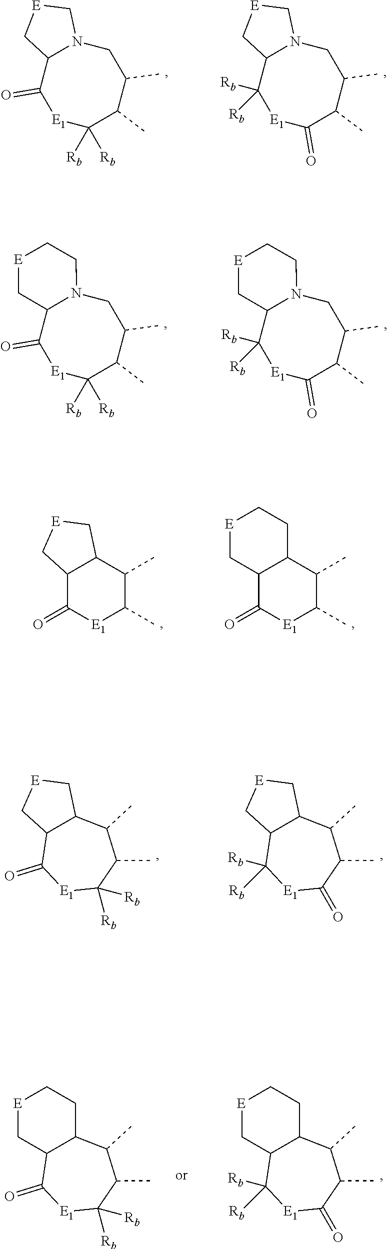

each R_{b} is independently selected from H, D, OH, halogen and C₁₋₃ alkyl, wherein the C₁₋₃ alkyl is optionally substituted with 1, 2 or 3 halogens, and other variables are as defined in the present disclosure.

In some embodiments of the present disclosure, when the above T is CH or N, R₃ and R₄ are taken together with the carbon atoms to which they are attached to form

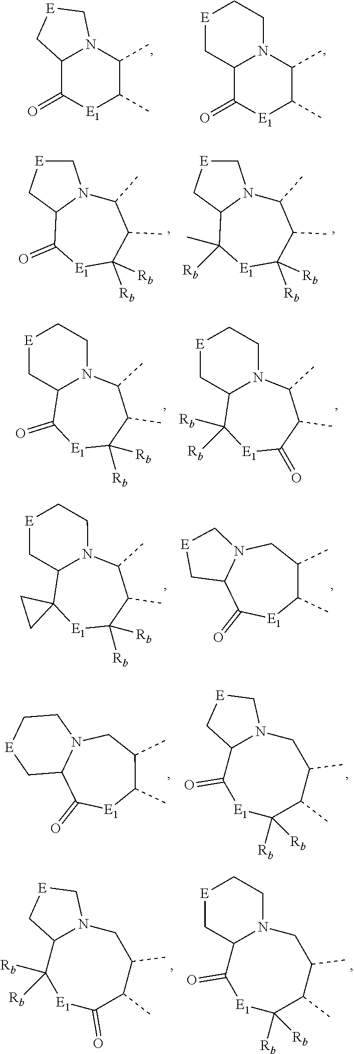

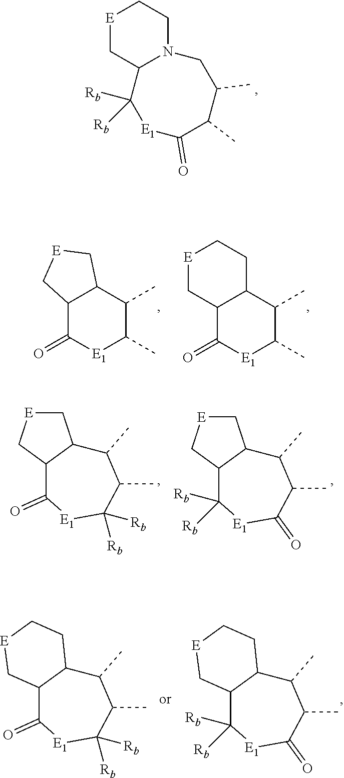

and other variables are as defined in the present disclosure.

In some embodiments of the present disclosure, when the above T is CH or N, R₃ and R₄ are taken together with the carbon atoms to which they are attached to form

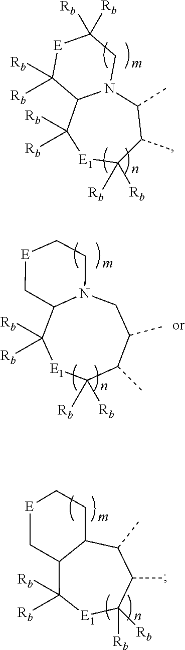

or, when T is CH or N, R₃ and R₄ are taken together with the carbon atoms to which they are attached to form

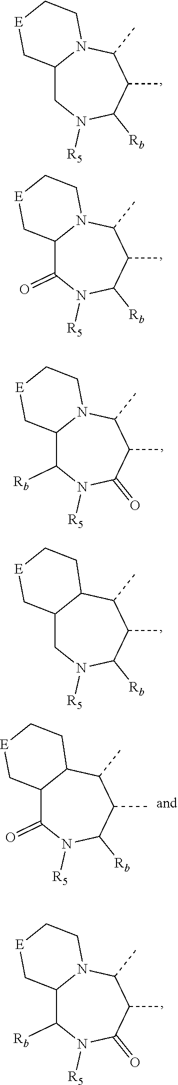

and other variables are as defined in the present disclosure.

In some embodiments of the present disclosure, when the above T is CH or N, R₃ and R₄ are taken together with the carbon atoms to which they are attached to form

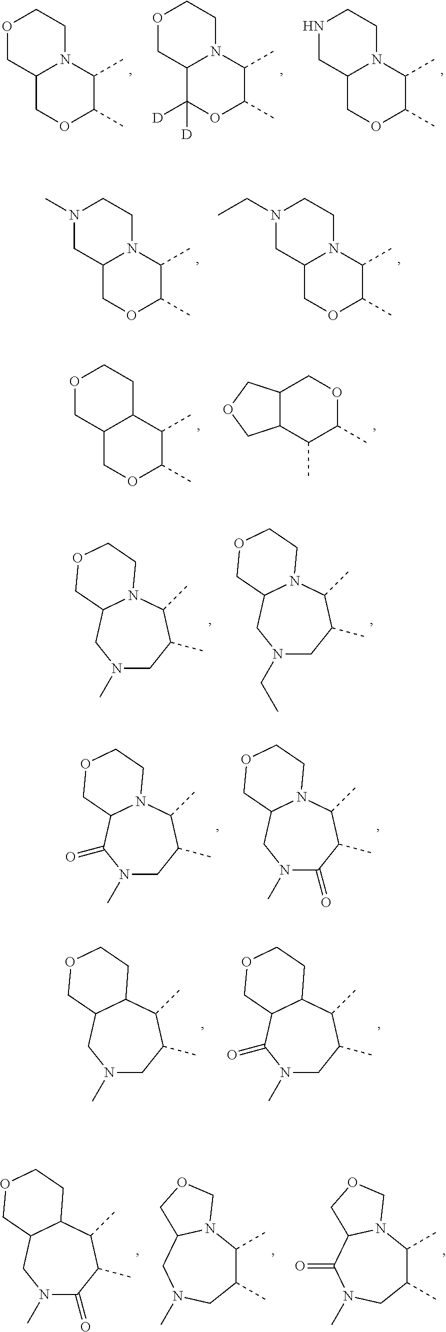

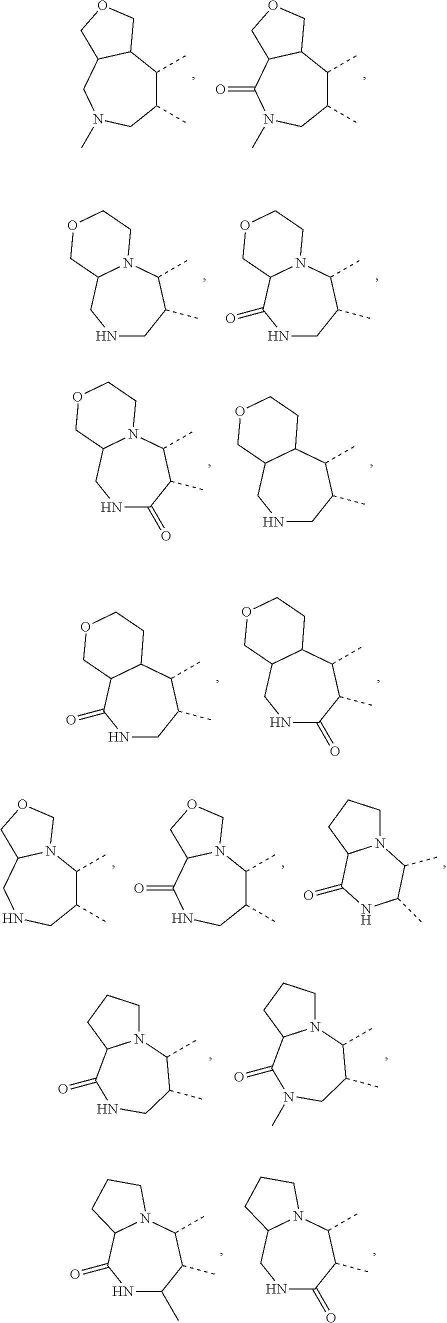

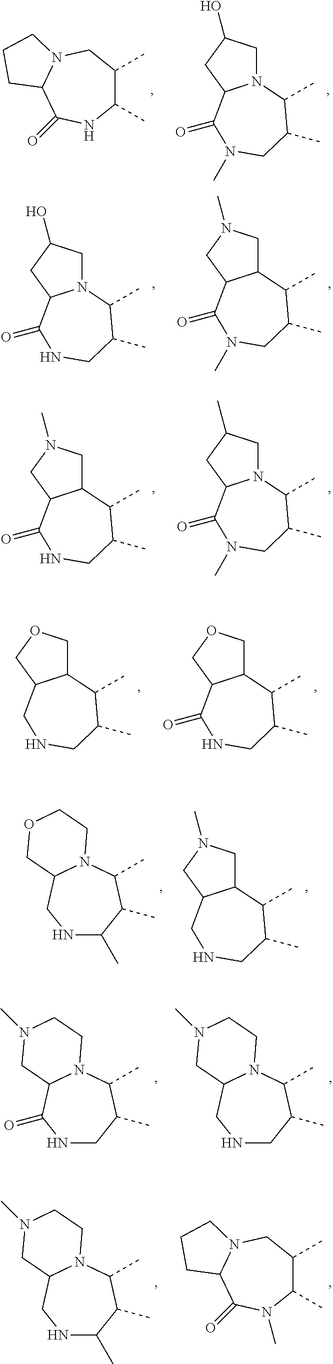

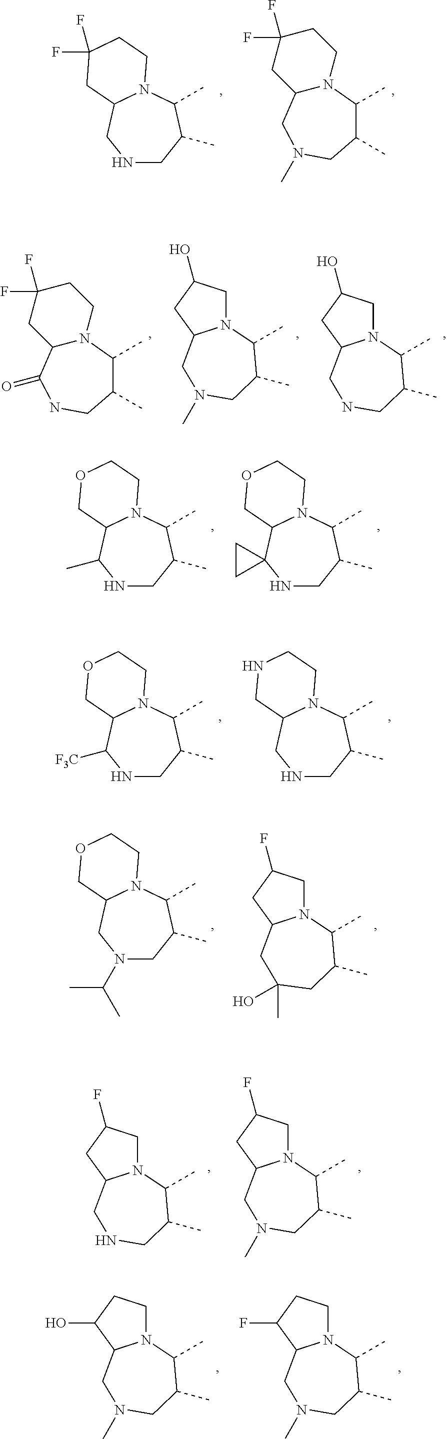

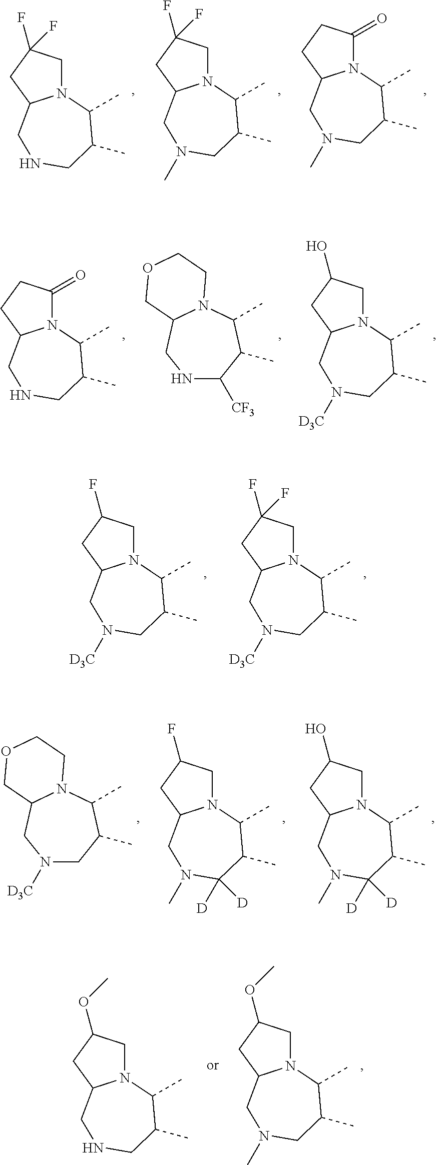

and other variables are as defined in the present disclosure.

In some embodiments of the present disclosure, when the above T is CH or N, R₃ and R₄ are taken together with the carbon atoms to which they are attached to form

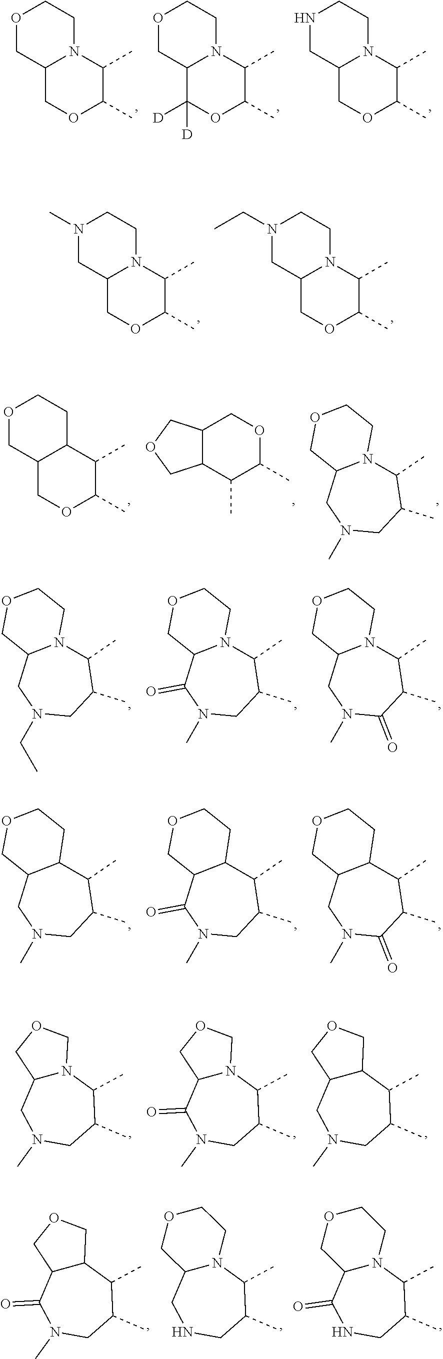

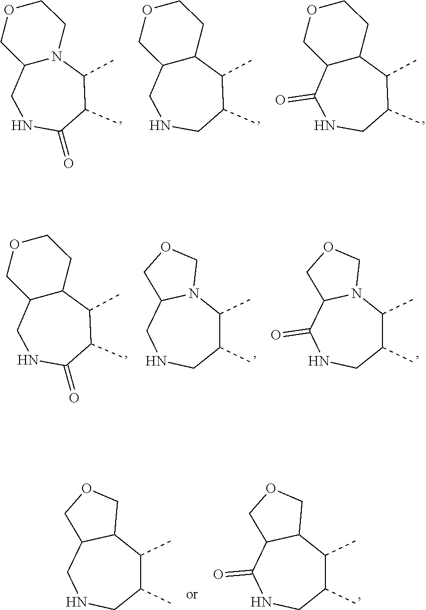

and other variables are as defined in the present disclosure.

In some embodiments of the present disclosure, when the above T is CH or N, R₃ and R₄ are taken together with the carbon atoms to which they are attached to form

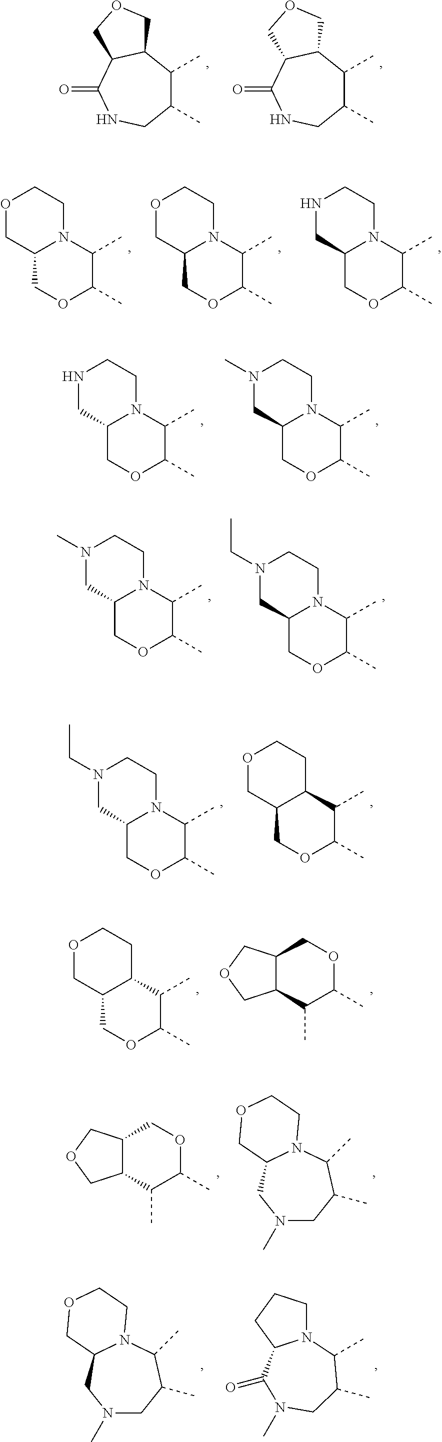

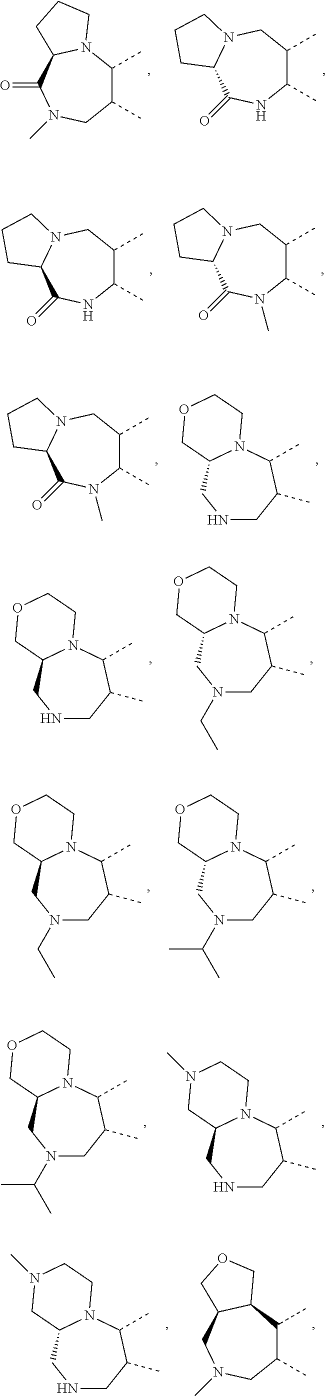

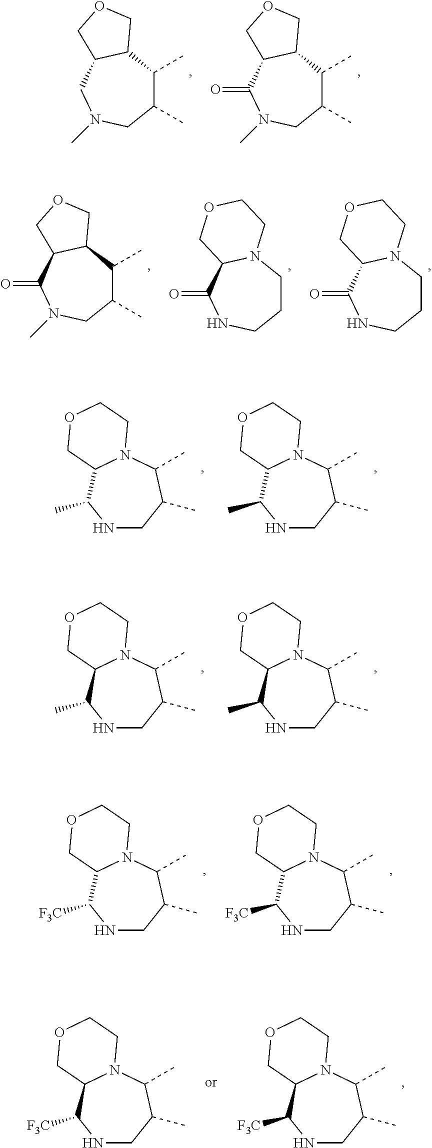

and other variables are as defined in the present disclosure.

In some embodiments of the present disclosure, when the above T is CH or N, R₃ and R₄ are taken together with the carbon atoms to which they are attached to form

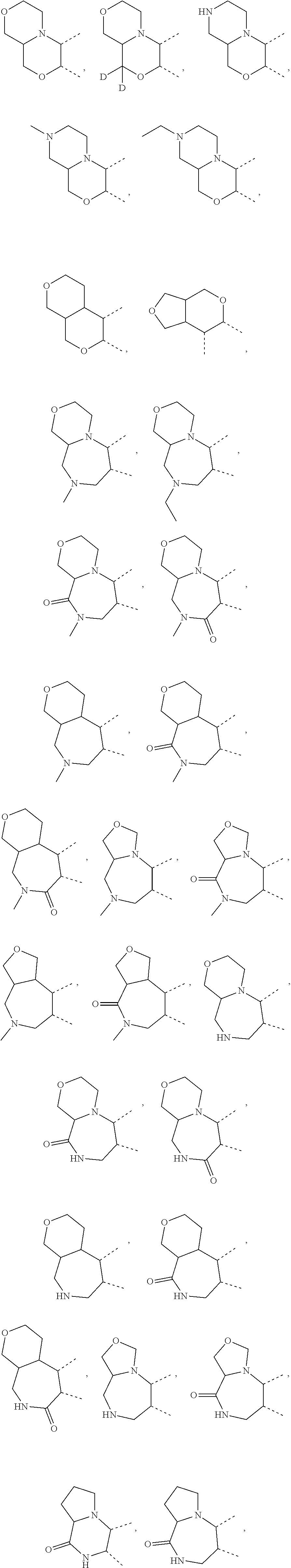

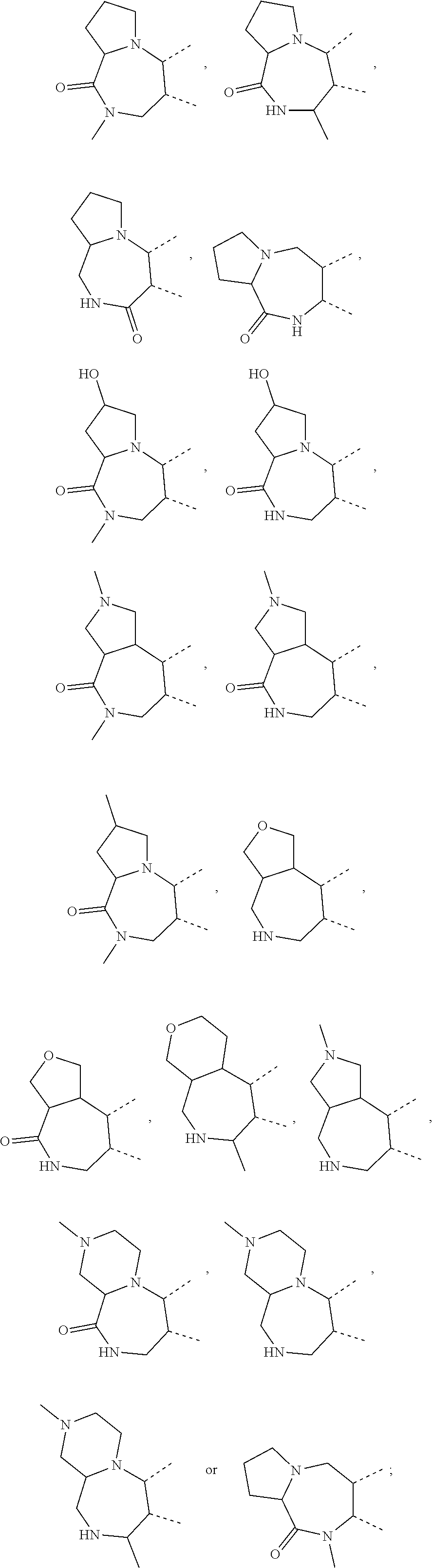

or, when T is CH or N, R₃ and R₄ are taken together with the carbon atoms to which they are attached to form

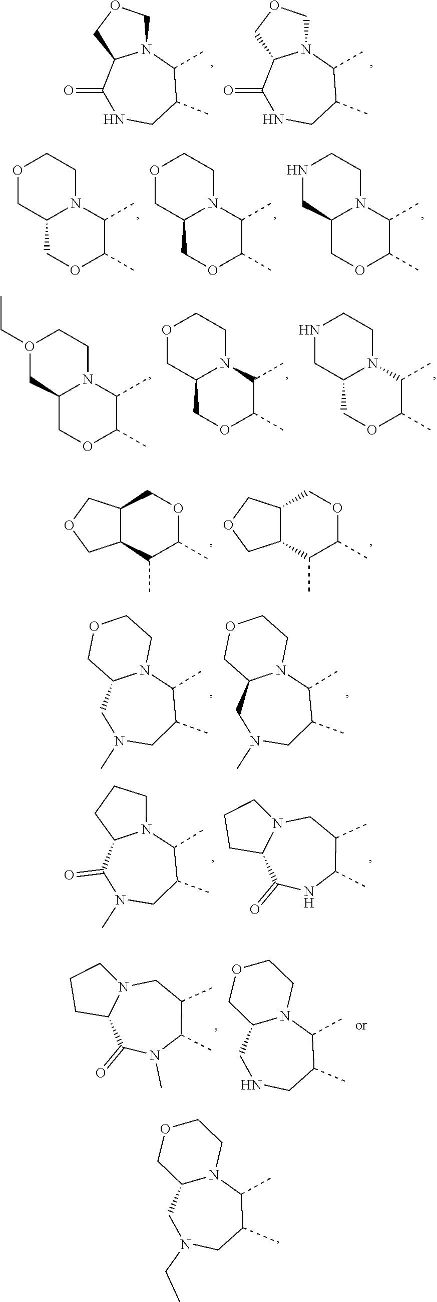

and other variables are as defined in the present disclosure.

In some embodiments of the present disclosure, when the above T is CF or N, R₃ is

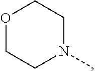

R₄ is —CH₂—N(CH₃)₂, and the other variables are as defined in the present disclosure.

Some other embodiments of the present disclosure are obtained by any combination of the above variables.

In some embodiments of the present disclosure, disclosed herein is the above compound, a stereoisomer or a pharmaceutically acceptable salt thereof, which is selected from,

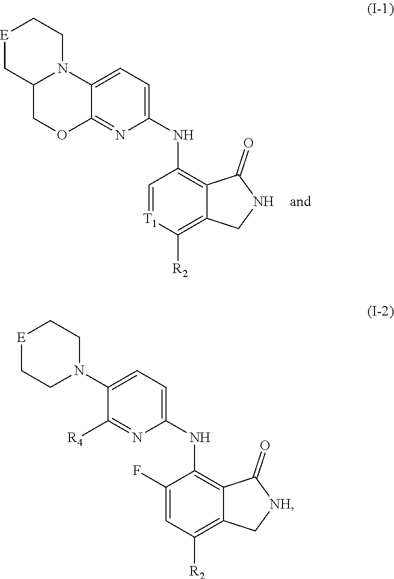

wherein E, T₁, R₂ and R₄ are as defined in the present disclosure.

In some embodiments of the present disclosure, disclosed herein is the above compound, a stereoisomer or a pharmaceutically acceptable salt thereof, which is selected from,

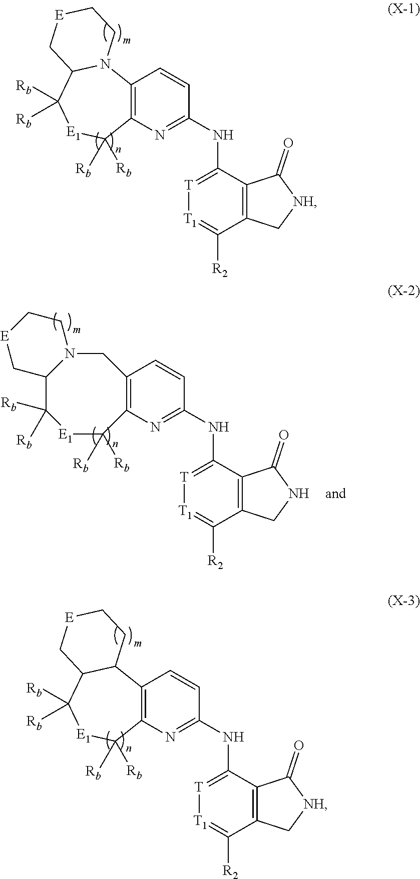

wherein, E, E₁, T, T₁, R₂, R_{b}, m and n are as defined in the present disclosure.

In some embodiments of the present disclosure, disclosed herein is the above compound, a stereoisomer or a pharmaceutically acceptable salt thereof, which is selected from:

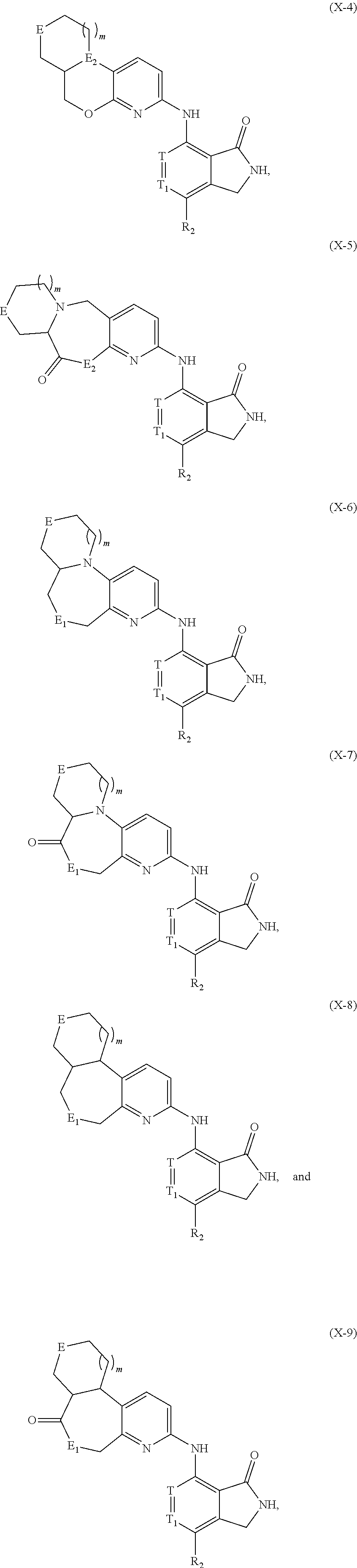

wherein, E, E₁, T, T₁, R₂ and m are as defined in the present disclosure.
E₂ is selected from N and CH.

In some embodiments of the present disclosure, disclosed herein is the above compound, a stereoisomer or a pharmaceutically acceptable salt thereof, which is selected from:

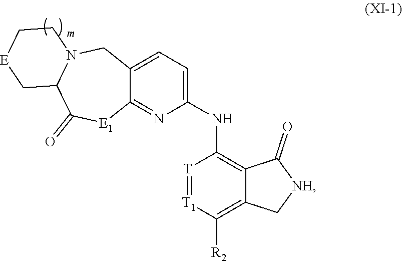

wherein, E, E₁, T, T₁, R₂ and m are as defined in the present disclosure.

The present disclosure also provides a compound represented by the following formula, a stereoisomer or a pharmaceutically acceptable salt thereof,

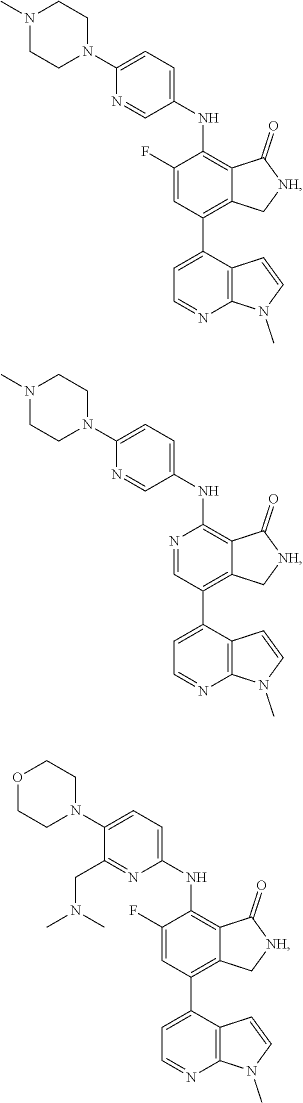 [SMILES: CN1CCN(c2ccc(Nc3c(F)cc(-c4ccnc5c4ccn5C)c4c3C(=O)NC4)cn2)CC1 ; CN1CCN(c2ccc(Nc3ncc(-c4ccnc5c4ccn5C)c4c3C(=O)NC4)cn2)CC1 ; CN(C)Cc1nc(Nc2c(F)cc(-c3ccnc4c3ccn4C)c3c2C(=O)NC3)ccc1N1CCOCC1]

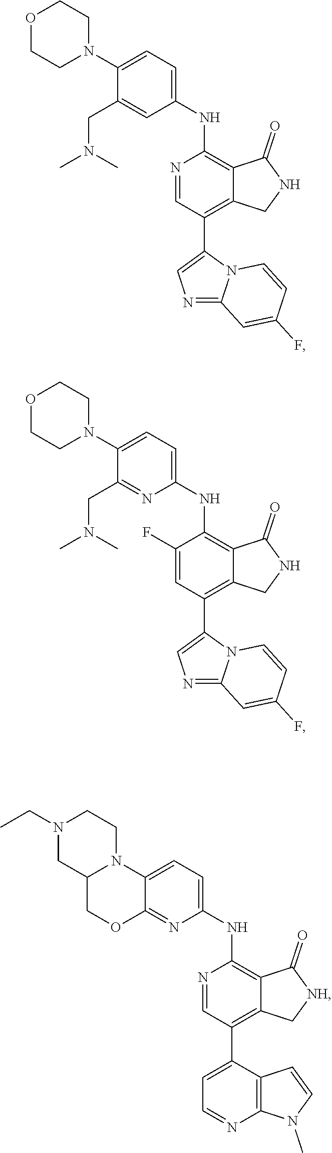 [SMILES: CN(C)Cc1cc(Nc2ncc(-c3cnc4cc(F)ccn34)c3c2C(=O)NC3)ccc1N1CCOCC1 ; CN(C)Cc1nc(Nc2c(F)cc(-c3cnc4cc(F)ccn34)c3c2C(=O)NC3)ccc1N1CCOCC1 ; CCN1CCN2c3ccc(Nc4ncc(-c5ccnc6c5ccn6C)c5c4C(=O)NC5)nc3OCC2C1]

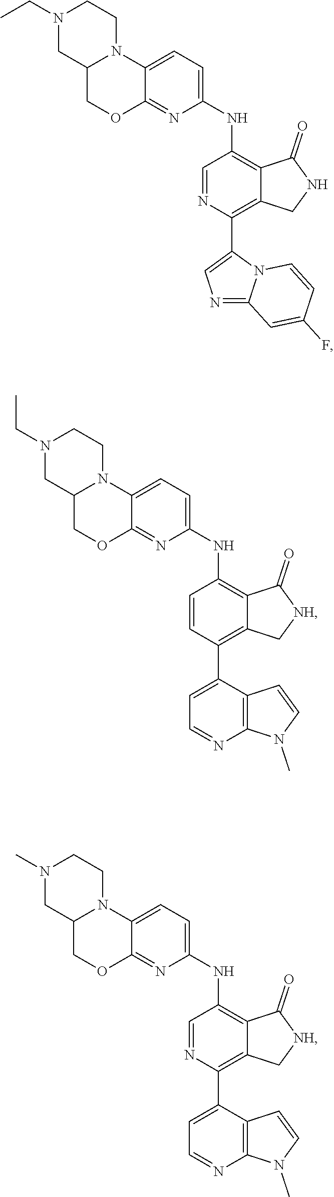 [SMILES: CCN1CCN2c3ccc(Nc4cnc(-c5cnc6cc(F)ccn56)c5c4C(=O)NC5)nc3OCC2C1 ; CCN1CCN2c3ccc(Nc4ccc(-c5ccnc6c5ccn6C)c5c4C(=O)NC5)nc3OCC2C1 ; CN1CCN2c3ccc(Nc4cnc(-c5ccnc6c5ccn6C)c5c4C(=O)NC5)nc3OCC2C1]

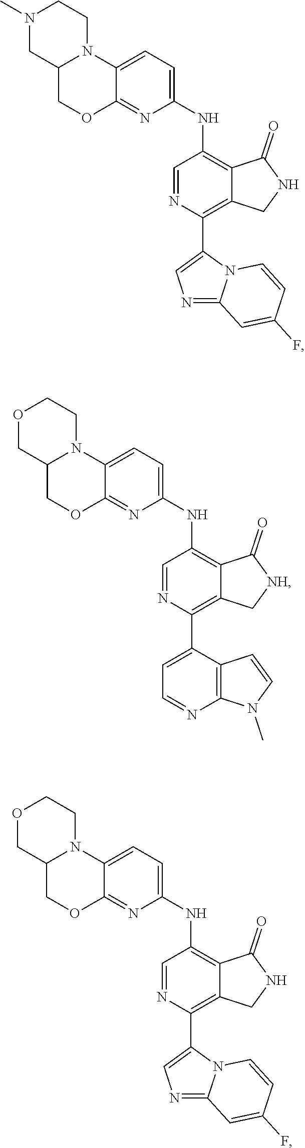 [SMILES: CN1CCN2c3ccc(Nc4cnc(-c5cnc6cc(F)ccn56)c5c4C(=O)NC5)nc3OCC2C1 ; Cn1ccc2c(-c3ncc(Nc4ccc5c(n4)OCC4COCCN54)c4c3CNC4=O)ccnc21 ; O=C1NCc2c(-c3cnc4cc(F)ccn34)ncc(Nc3ccc4c(n3)OCC3COCCN43)c21]

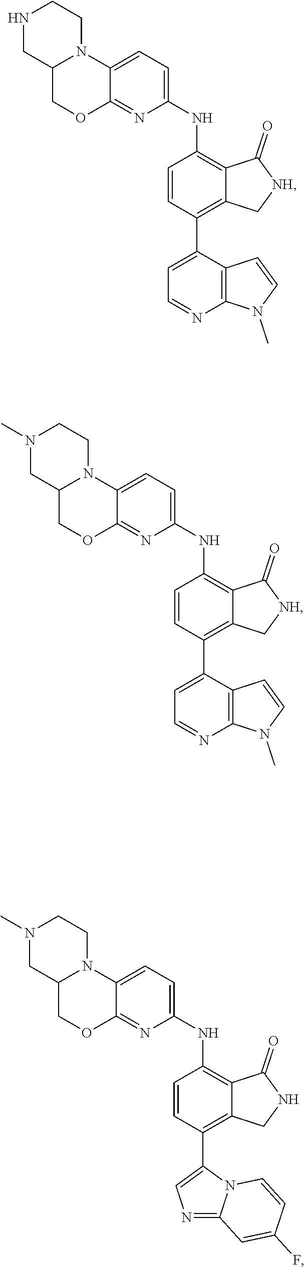 [SMILES: Cn1ccc2c(-c3ccc(Nc4ccc5c(n4)OCC4CNCCN54)c4c3CNC4=O)ccnc21 ; CN1CCN2c3ccc(Nc4ccc(-c5ccnc6c5ccn6C)c5c4C(=O)NC5)nc3OCC2C1 ; CN1CCN2c3ccc(Nc4ccc(-c5cnc6cc(F)ccn56)c5c4C(=O)NC5)nc3OCC2C1]

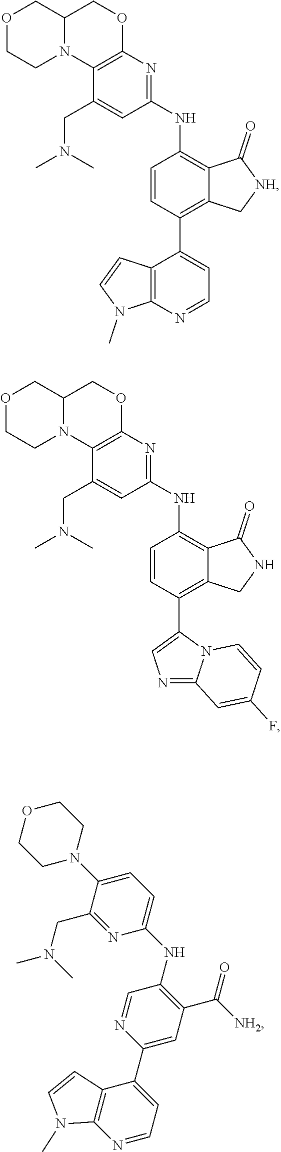

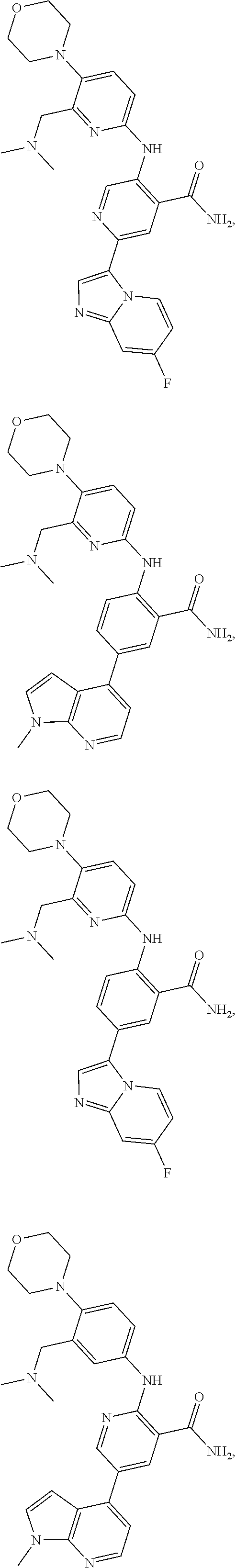 [SMILES: CN(C)Cc1nc(Nc2cnc(-c3cnc4cc(F)ccn34)cc2C(N)=O)ccc1N1CCOCC1 ; CN(C)Cc1nc(Nc2ccc(-c3ccnc4c3ccn4C)cc2C(N)=O)ccc1N1CCOCC1 ; CN(C)Cc1nc(Nc2ccc(-c3cnc4cc(F)ccn34)cc2C(N)=O)ccc1N1CCOCC1 ; CN(C)Cc1cc(Nc2ncc(-c3ccnc4c3ccn4C)cc2C(N)=O)ccc1N1CCOCC1]

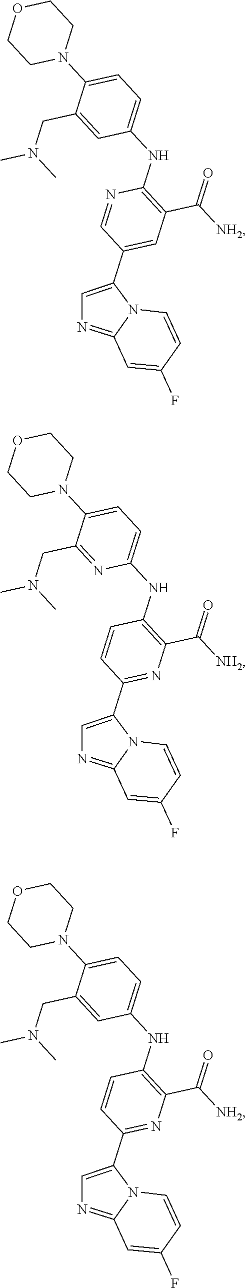 [SMILES: CN(C)Cc1cc(Nc2ncc(-c3cnc4cc(F)ccn34)cc2C(N)=O)ccc1N1CCOCC1 ; CN(C)Cc1nc(Nc2ccc(-c3cnc4cc(F)ccn34)nc2C(N)=O)ccc1N1CCOCC1 ; CN(C)Cc1cc(Nc2ccc(-c3cnc4cc(F)ccn34)nc2C(N)=O)ccc1N1CCOCC1]

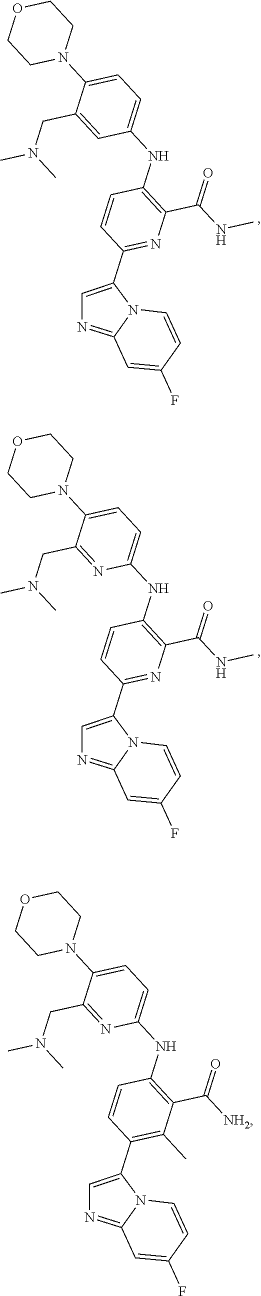

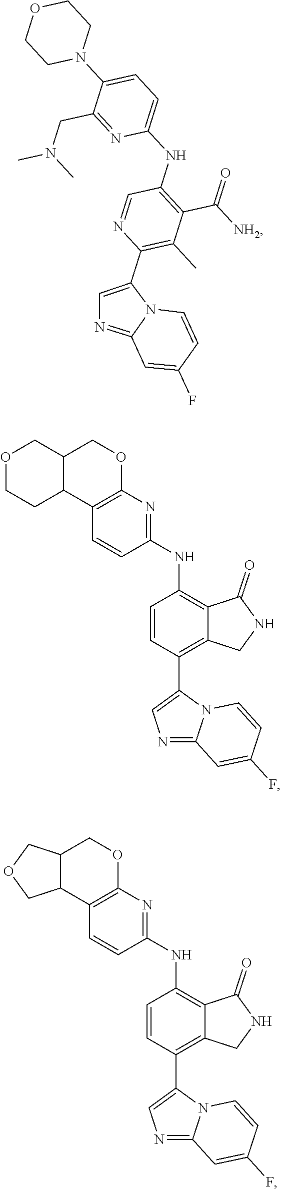 [SMILES: Cc1c(-c2cnc3cc(F)ccn23)ncc(Nc2ccc(N3CCOCC3)c(CN(C)C)n2)c1C(N)=O ; O=C1NCc2c(-c3cnc4cc(F)ccn34)ccc(Nc3ccc4c(n3)OCC3COCCC43)c21 ; O=C1NCc2c(-c3cnc4cc(F)ccn34)ccc(Nc3ccc4c(n3)OCC3COCC43)c21]

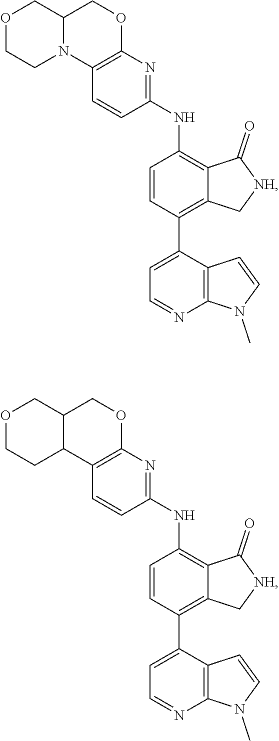 [SMILES: Cn1ccc2c(-c3ccc(Nc4ccc5c(n4)OCC4COCCN54)c4c3CNC4=O)ccnc21 ; Cn1ccc2c(-c3ccc(Nc4ccc5c(n4)OCC4COCCC54)c4c3CNC4=O)ccnc21]

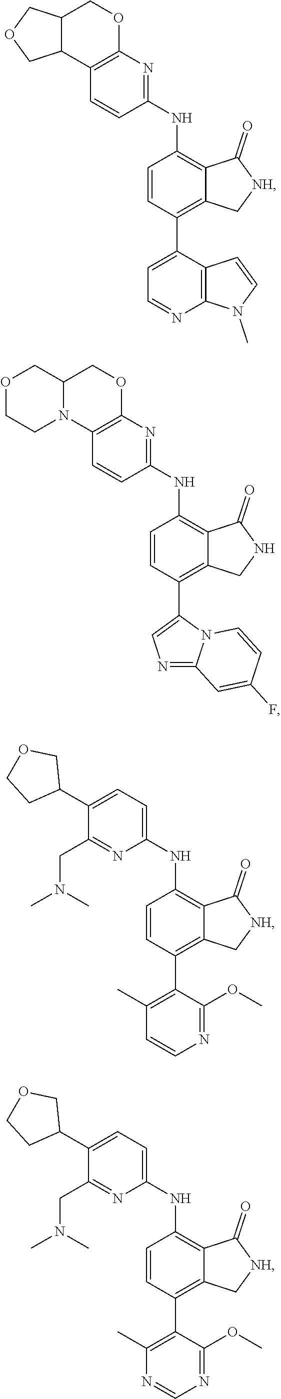 [SMILES: Cn1ccc2c(-c3ccc(Nc4ccc5c(n4)OCC4COCC54)c4c3CNC4=O)ccnc21 ; O=C1NCc2c(-c3cnc4cc(F)ccn34)ccc(Nc3ccc4c(n3)OCC3COCCN43)c21 ; COc1nccc(C)c1-c1ccc(Nc2ccc(C3CCOC3)c(CN(C)C)n2)c2c1CNC2=O ; COc1ncnc(C)c1-c1ccc(Nc2ccc(C3CCOC3)c(CN(C)C)n2)c2c1CNC2=O]

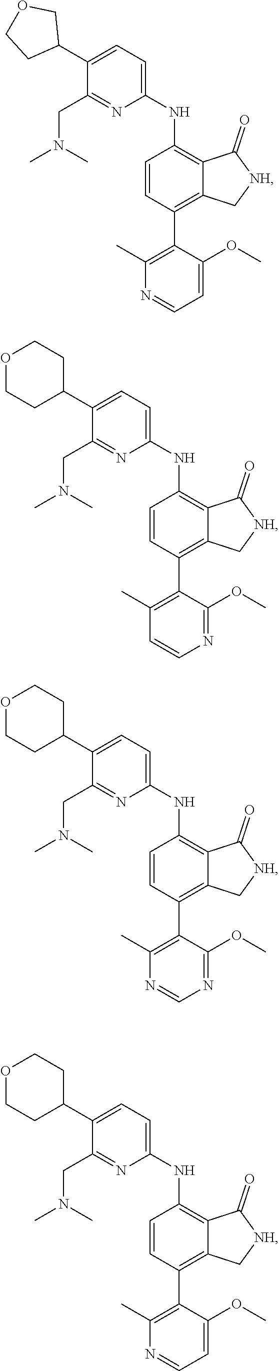 [SMILES: COc1ccnc(C)c1-c1ccc(Nc2ccc(C3CCOC3)c(CN(C)C)n2)c2c1CNC2=O ; COc1nccc(C)c1-c1ccc(Nc2ccc(C3CCOCC3)c(CN(C)C)n2)c2c1CNC2=O ; COc1ncnc(C)c1-c1ccc(Nc2ccc(C3CCOCC3)c(CN(C)C)n2)c2c1CNC2=O ; COc1ccnc(C)c1-c1ccc(Nc2ccc(C3CCOCC3)c(CN(C)C)n2)c2c1CNC2=O]

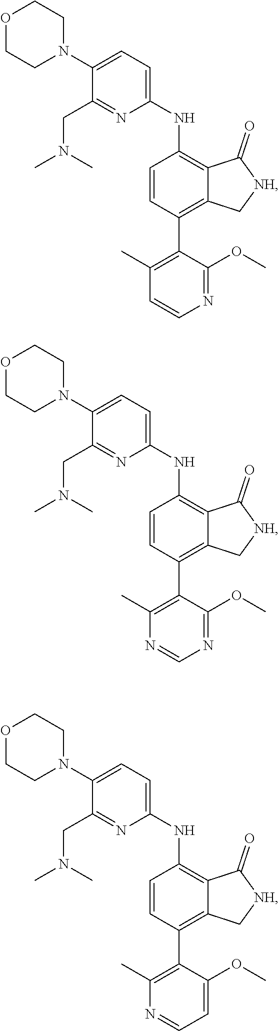 [SMILES: COc1nccc(C)c1-c1ccc(Nc2ccc(N3CCOCC3)c(CN(C)C)n2)c2c1CNC2=O ; COc1ncnc(C)c1-c1ccc(Nc2ccc(N3CCOCC3)c(CN(C)C)n2)c2c1CNC2=O ; COc1ccnc(C)c1-c1ccc(Nc2ccc(N3CCOCC3)c(CN(C)C)n2)c2c1CNC2=O]

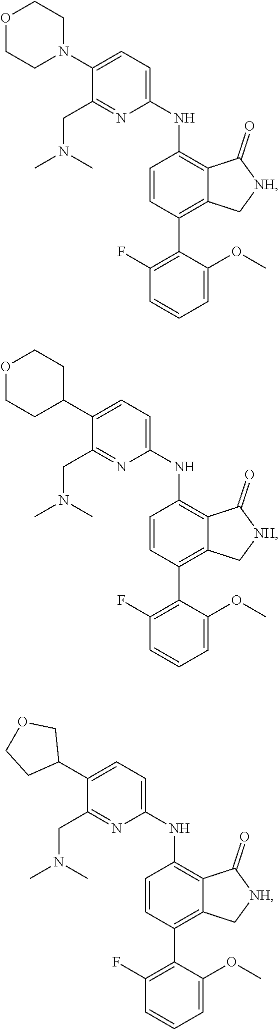 [SMILES: COc1cccc(F)c1-c1ccc(Nc2ccc(N3CCOCC3)c(CN(C)C)n2)c2c1CNC2=O ; COc1cccc(F)c1-c1ccc(Nc2ccc(C3CCOCC3)c(CN(C)C)n2)c2c1CNC2=O ; COc1cccc(F)c1-c1ccc(Nc2ccc(C3CCOC3)c(CN(C)C)n2)c2c1CNC2=O]

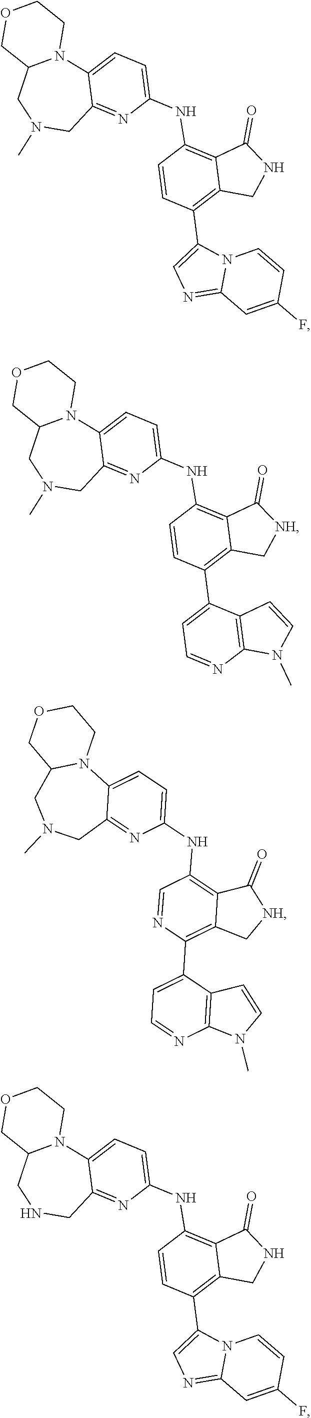 [SMILES: CN1Cc2nc(Nc3ccc(-c4cnc5cc(F)ccn45)c4c3C(=O)NC4)ccc2N2CCOCC2C1 ; CN1Cc2nc(Nc3ccc(-c4ccnc5c4ccn5C)c4c3C(=O)NC4)ccc2N2CCOCC2C1 ; CN1Cc2nc(Nc3cnc(-c4ccnc5c4ccn5C)c4c3C(=O)NC4)ccc2N2CCOCC2C1 ; O=C1NCc2c(-c3cnc4cc(F)ccn34)ccc(Nc3ccc4c(n3)CNCC3COCCN43)c21]

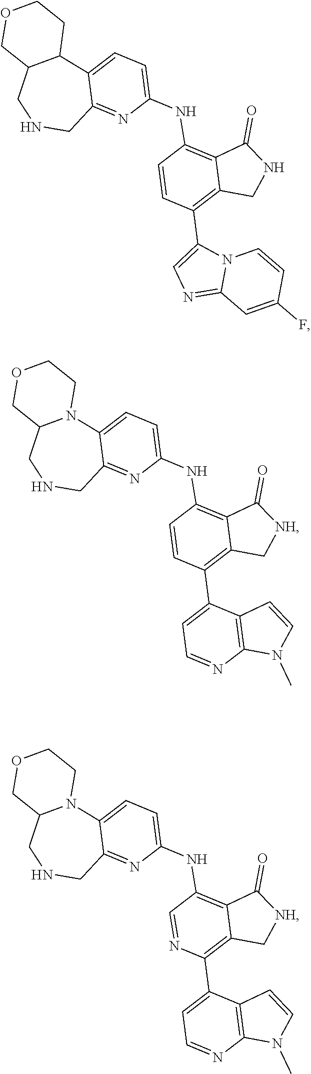 [SMILES: O=C1NCc2c(-c3cnc4cc(F)ccn34)ccc(Nc3ccc4c(n3)CNCC3COCCC43)c21 ; Cn1ccc2c(-c3ccc(Nc4ccc5c(n4)CNCC4COCCN54)c4c3CNC4=O)ccnc21 ; Cn1ccc2c(-c3ncc(Nc4ccc5c(n4)CNCC4COCCN54)c4c3CNC4=O)ccnc21]

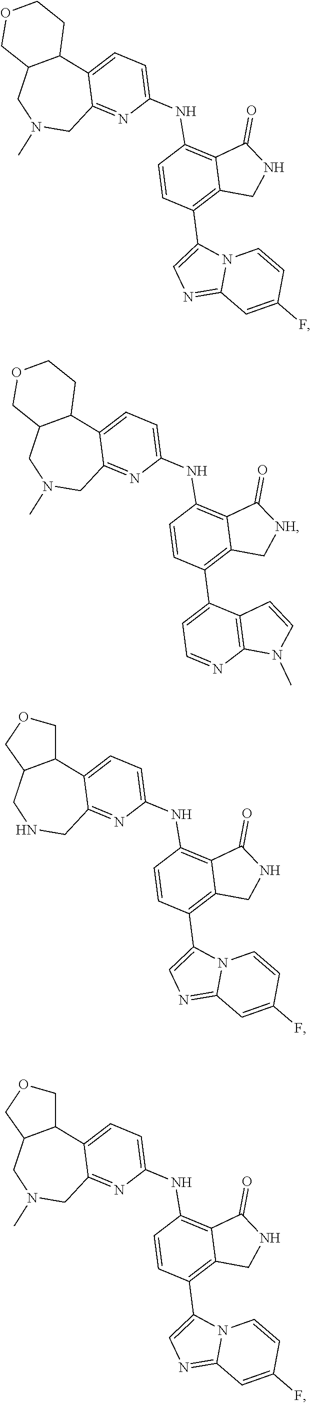 [SMILES: CN1Cc2nc(Nc3ccc(-c4cnc5cc(F)ccn45)c4c3C(=O)NC4)ccc2C2CCOCC2C1 ; CN1Cc2nc(Nc3ccc(-c4ccnc5c4ccn5C)c4c3C(=O)NC4)ccc2C2CCOCC2C1 ; O=C1NCc2c(-c3cnc4cc(F)ccn34)ccc(Nc3ccc4c(n3)CNCC3COCC43)c21 ; CN1Cc2nc(Nc3ccc(-c4cnc5cc(F)ccn45)c4c3C(=O)NC4)ccc2C2COCC2C1]

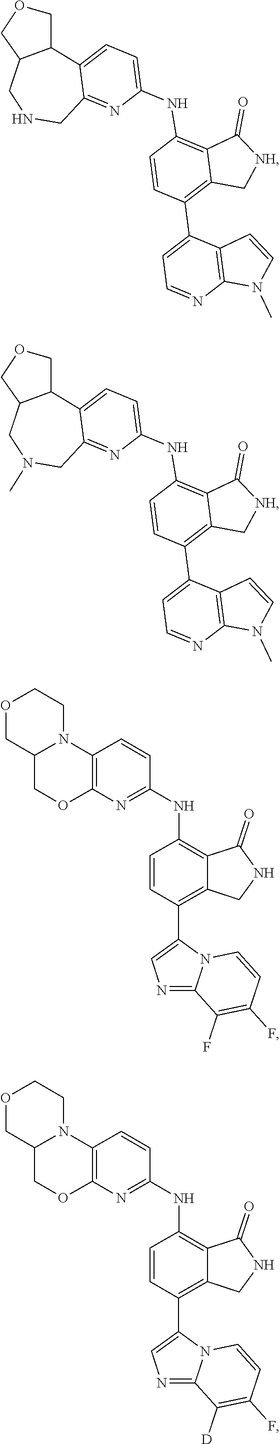 [SMILES: Cn1ccc2c(-c3ccc(Nc4ccc5c(n4)CNCC4COCC54)c4c3CNC4=O)ccnc21 ; CN1Cc2nc(Nc3ccc(-c4ccnc5c4ccn5C)c4c3C(=O)NC4)ccc2C2COCC2C1 ; O=C1NCc2c(-c3cnc4c(F)c(F)ccn34)ccc(Nc3ccc4c(n3)OCC3COCCN43)c21 ; [2H]c1c(F)ccn2c(-c3ccc(Nc4ccc5c(n4)OCC4COCCN54)c4c3CNC4=O)cnc12]

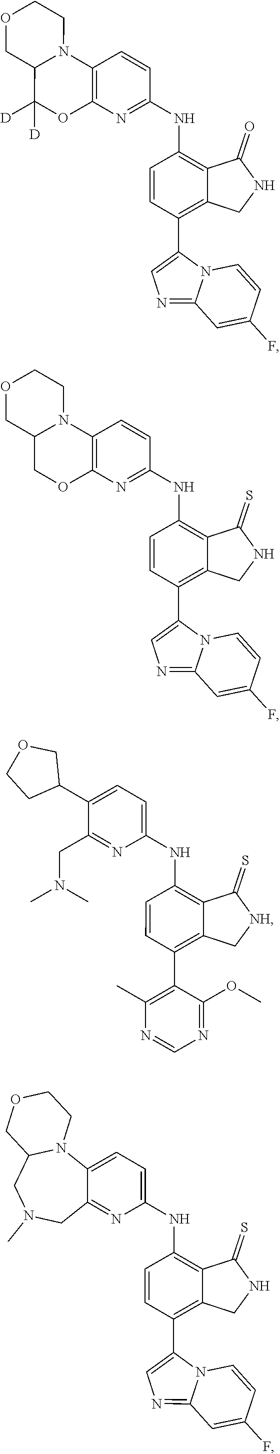 [SMILES: [2H]C1([2H])Oc2nc(Nc3ccc(-c4cnc5cc(F)ccn45)c4c3C(=O)NC4)ccc2N2CCOCC21 ; Fc1ccn2c(-c3ccc(Nc4ccc5c(n4)OCC4COCCN54)c4c3CNC4=S)cnc2c1 ; COc1ncnc(C)c1-c1ccc(Nc2ccc(C3CCOC3)c(CN(C)C)n2)c2c1CNC2=S ; CN1Cc2nc(Nc3ccc(-c4cnc5cc(F)ccn45)c4c3C(=S)NC4)ccc2N2CCOCC2C1]

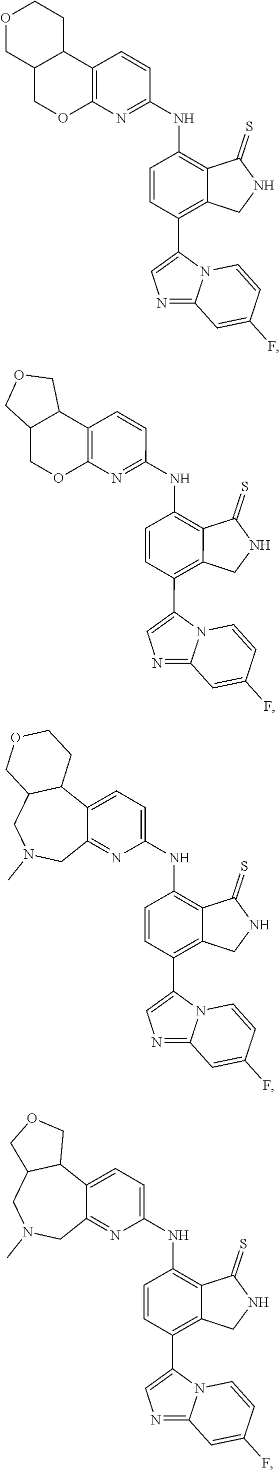 [SMILES: Fc1ccn2c(-c3ccc(Nc4ccc5c(n4)OCC4COCCC54)c4c3CNC4=S)cnc2c1 ; Fc1ccn2c(-c3ccc(Nc4ccc5c(n4)OCC4COCC54)c4c3CNC4=S)cnc2c1 ; CN1Cc2nc(Nc3ccc(-c4cnc5cc(F)ccn45)c4c3C(=S)NC4)ccc2C2CCOCC2C1 ; CN1Cc2nc(Nc3ccc(-c4cnc5cc(F)ccn45)c4c3C(=S)NC4)ccc2C2COCC2C1]

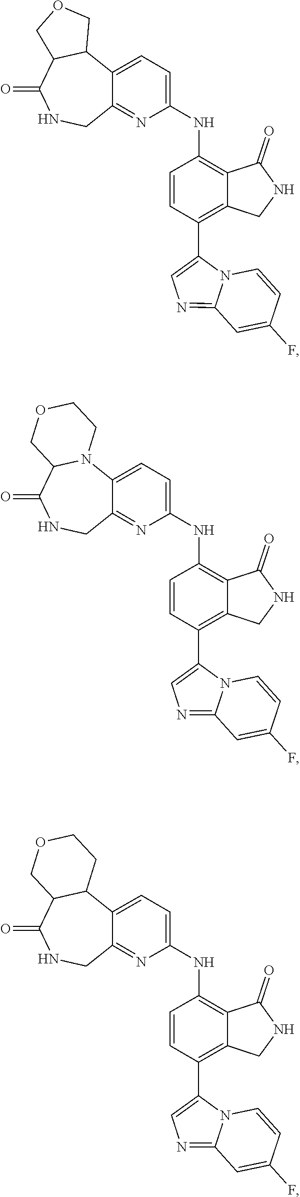 [SMILES: O=C1NCc2c(-c3cnc4cc(F)ccn34)ccc(Nc3ccc4c(n3)CNC(=O)C3COCC43)c21 ; O=C1NCc2c(-c3cnc4cc(F)ccn34)ccc(Nc3ccc4c(n3)CNC(=O)C3COCCN43)c21 ; O=C1NCc2c(-c3cnc4cc(F)ccn34)ccc(Nc3ccc4c(n3)CNC(=O)C3COCCC43)c21]

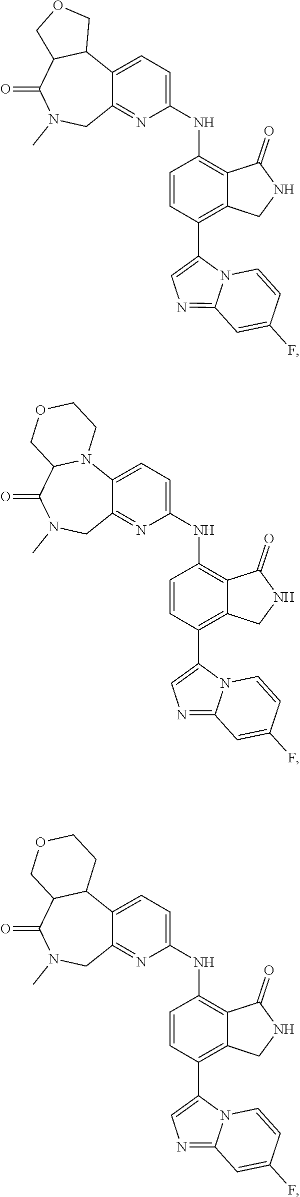 [SMILES: CN1Cc2nc(Nc3ccc(-c4cnc5cc(F)ccn45)c4c3C(=O)NC4)ccc2C2COCC2C1=O ; CN1Cc2nc(Nc3ccc(-c4cnc5cc(F)ccn45)c4c3C(=O)NC4)ccc2N2CCOCC2C1=O ; CN1Cc2nc(Nc3ccc(-c4cnc5cc(F)ccn45)c4c3C(=O)NC4)ccc2C2CCOCC2C1=O]

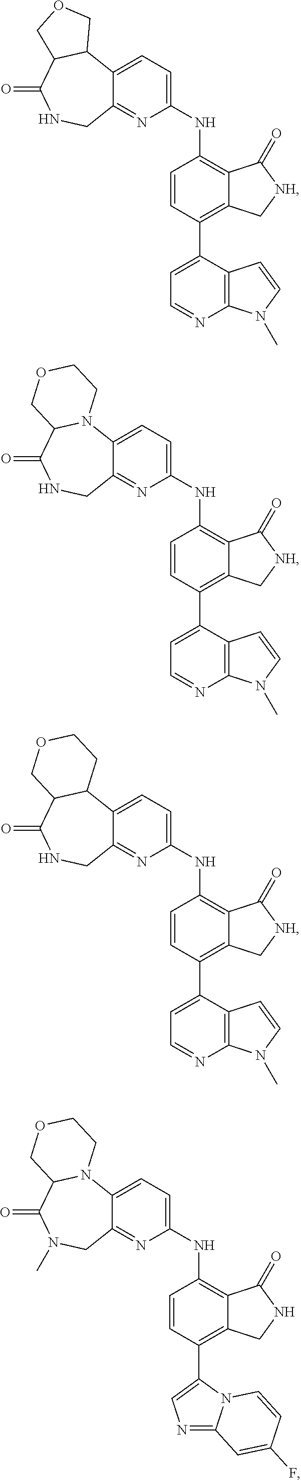 [SMILES: Cn1ccc2c(-c3ccc(Nc4ccc5c(n4)CNC(=O)C4COCC54)c4c3CNC4=O)ccnc21 ; Cn1ccc2c(-c3ccc(Nc4ccc5c(n4)CNC(=O)C4COCCN54)c4c3CNC4=O)ccnc21 ; Cn1ccc2c(-c3ccc(Nc4ccc5c(n4)CNC(=O)C4COCCC54)c4c3CNC4=O)ccnc21 ; CN1Cc2nc(Nc3ccc(-c4cnc5cc(F)ccn45)c4c3C(=O)NC4)ccc2N2CCOCC2C1=O]

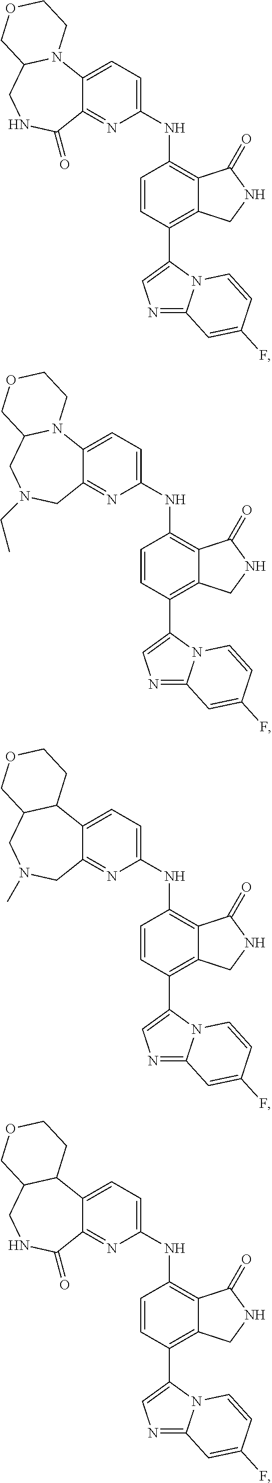 [SMILES: O=C1NCC2COCCN2c2ccc(Nc3ccc(-c4cnc5cc(F)ccn45)c4c3C(=O)NC4)nc21 ; CCN1Cc2nc(Nc3ccc(-c4cnc5cc(F)ccn45)c4c3C(=O)NC4)ccc2N2CCOCC2C1 ; CN1Cc2nc(Nc3ccc(-c4cnc5cc(F)ccn45)c4c3C(=O)NC4)ccc2C2CCOCC2C1 ; O=C1NCC2COCCC2c2ccc(Nc3ccc(-c4cnc5cc(F)ccn45)c4c3C(=O)NC4)nc21]

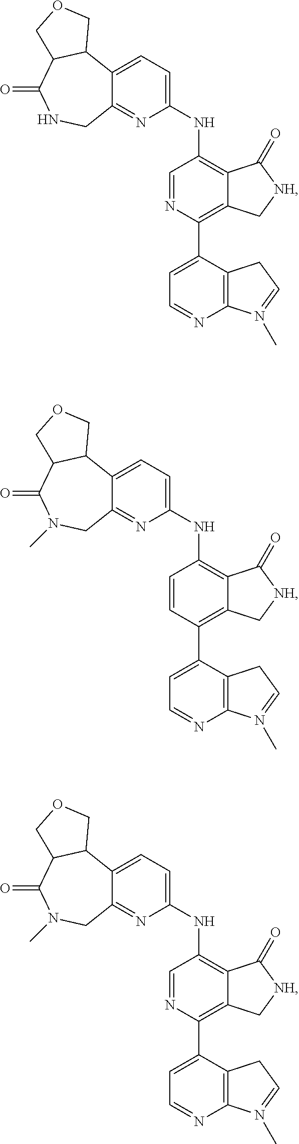

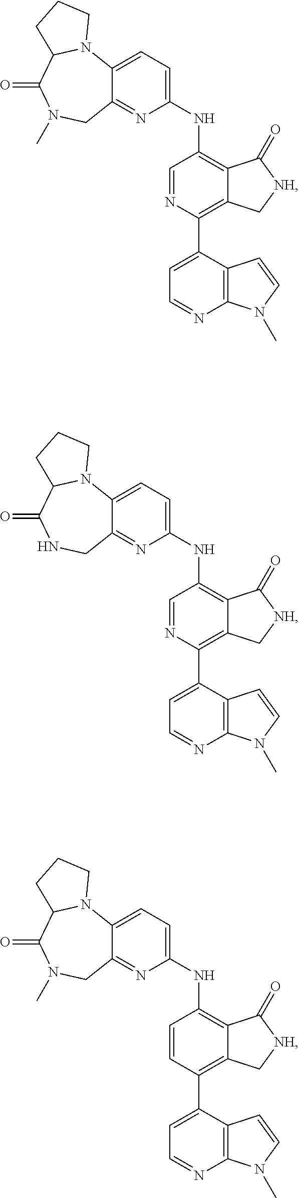 [SMILES: CN1Cc2nc(Nc3cnc(-c4ccnc5c4ccn5C)c4c3C(=O)NC4)ccc2N2CCCC2C1=O ; Cn1ccc2c(-c3ncc(Nc4ccc5c(n4)CNC(=O)C4CCCN54)c4c3CNC4=O)ccnc21 ; CN1Cc2nc(Nc3ccc(-c4ccnc5c4ccn5C)c4c3C(=O)NC4)ccc2N2CCCC2C1=O]

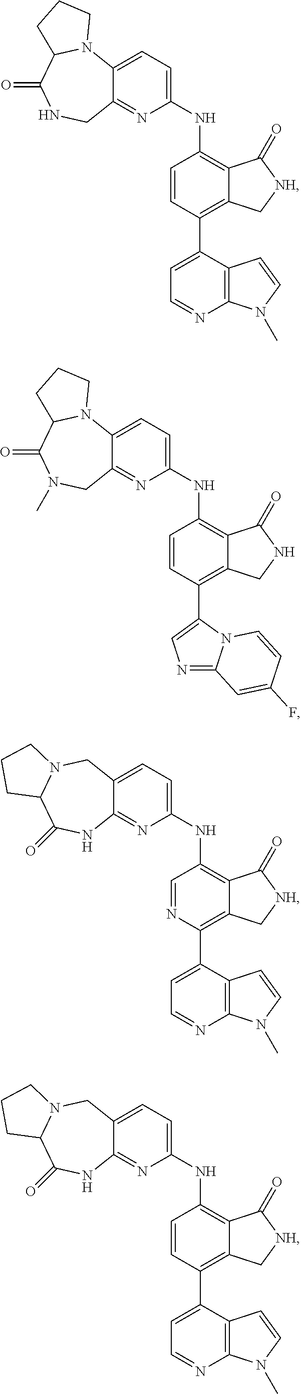 [SMILES: Cn1ccc2c(-c3ccc(Nc4ccc5c(n4)CNC(=O)C4CCCN54)c4c3CNC4=O)ccnc21 ; CN1Cc2nc(Nc3ccc(-c4cnc5cc(F)ccn45)c4c3C(=O)NC4)ccc2N2CCCC2C1=O ; Cn1ccc2c(-c3ncc(Nc4ccc5c(n4)NC(=O)C4CCCN4C5)c4c3CNC4=O)ccnc21 ; Cn1ccc2c(-c3ccc(Nc4ccc5c(n4)NC(=O)C4CCCN4C5)c4c3CNC4=O)ccnc21]

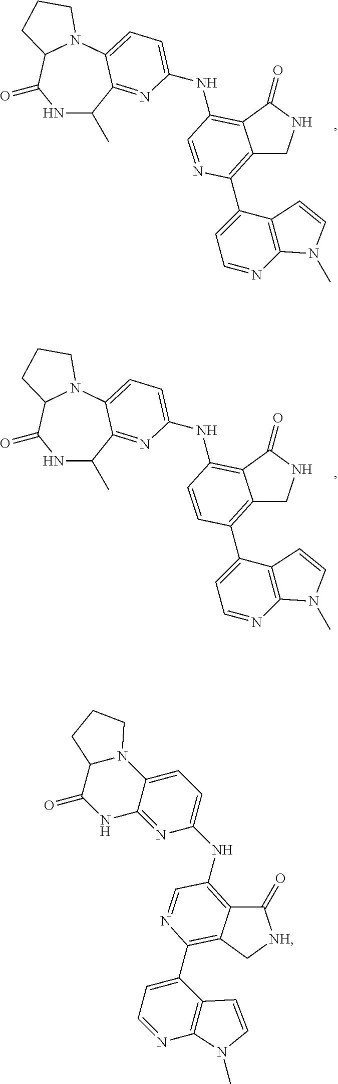 [SMILES: CC1NC(=O)C2CCCN2c2ccc(Nc3cnc(-c4ccnc5c4ccn5C)c4c3C(=O)NC4)nc21 ; CC1NC(=O)C2CCCN2c2ccc(Nc3ccc(-c4ccnc5c4ccn5C)c4c3C(=O)NC4)nc21 ; Cn1ccc2c(-c3ncc(Nc4ccc5c(n4)NC(=O)C4CCCN54)c4c3CNC4=O)ccnc21]

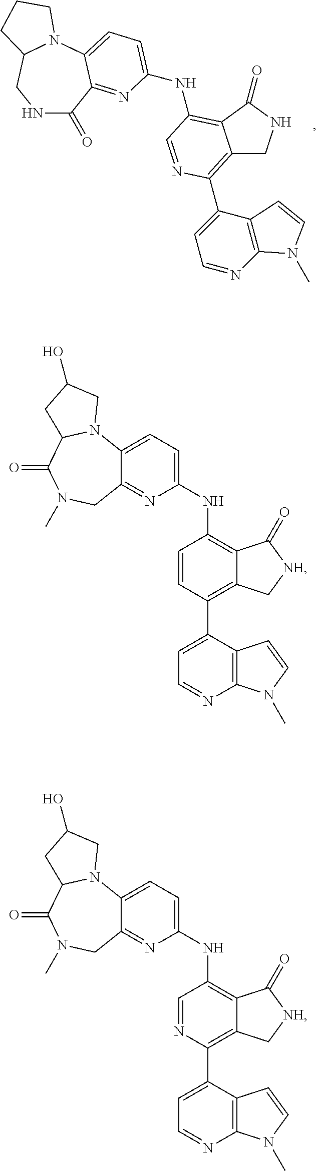 [SMILES: Cn1ccc2c(-c3ncc(Nc4ccc5c(n4)C(=O)NCC4CCCN54)c4c3CNC4=O)ccnc21 ; CN1Cc2nc(Nc3ccc(-c4ccnc5c4ccn5C)c4c3C(=O)NC4)ccc2N2CC(O)CC2C1=O ; CN1Cc2nc(Nc3cnc(-c4ccnc5c4ccn5C)c4c3C(=O)NC4)ccc2N2CC(O)CC2C1=O]

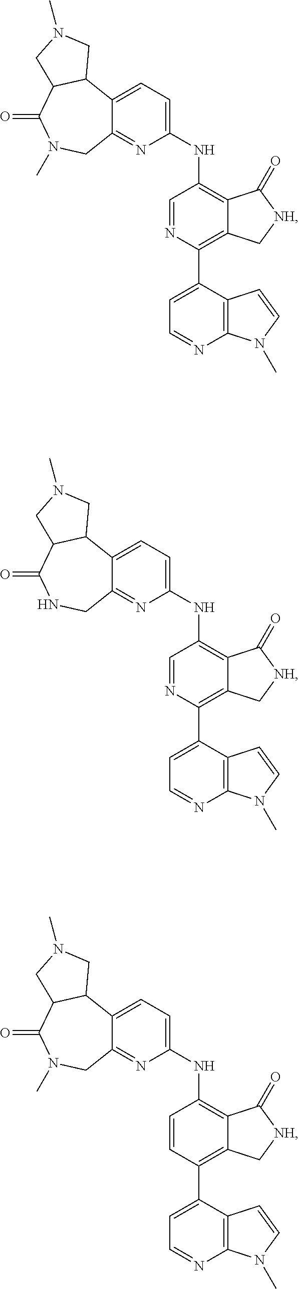 [SMILES: CN1CC2C(=O)N(C)Cc3nc(Nc4cnc(-c5ccnc6c5ccn6C)c5c4C(=O)NC5)ccc3C2C1 ; CN1CC2C(=O)NCc3nc(Nc4cnc(-c5ccnc6c5ccn6C)c5c4C(=O)NC5)ccc3C2C1 ; CN1CC2C(=O)N(C)Cc3nc(Nc4ccc(-c5ccnc6c5ccn6C)c5c4C(=O)NC5)ccc3C2C1]

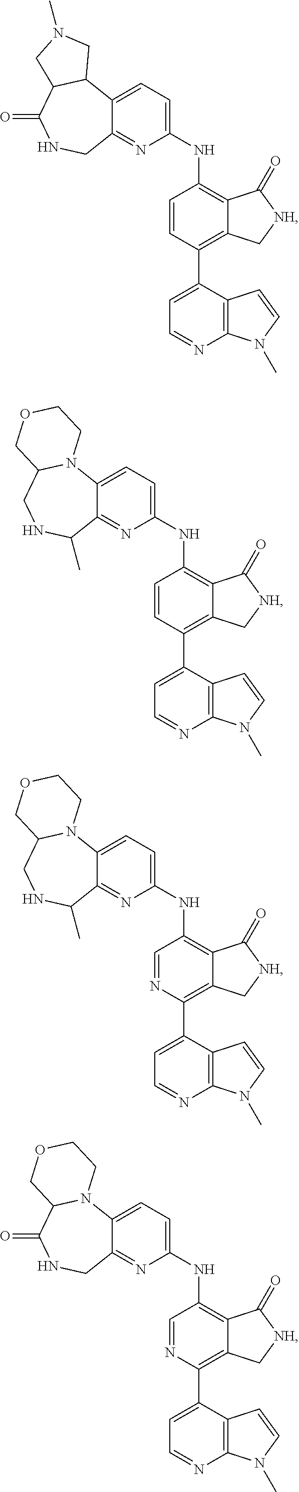 [SMILES: CN1CC2C(=O)NCc3nc(Nc4ccc(-c5ccnc6c5ccn6C)c5c4C(=O)NC5)ccc3C2C1 ; CC1NCC2COCCN2c2ccc(Nc3ccc(-c4ccnc5c4ccn5C)c4c3C(=O)NC4)nc21 ; CC1NCC2COCCN2c2ccc(Nc3cnc(-c4ccnc5c4ccn5C)c4c3C(=O)NC4)nc21 ; Cn1ccc2c(-c3ncc(Nc4ccc5c(n4)CNC(=O)C4COCCN54)c4c3CNC4=O)ccnc21]

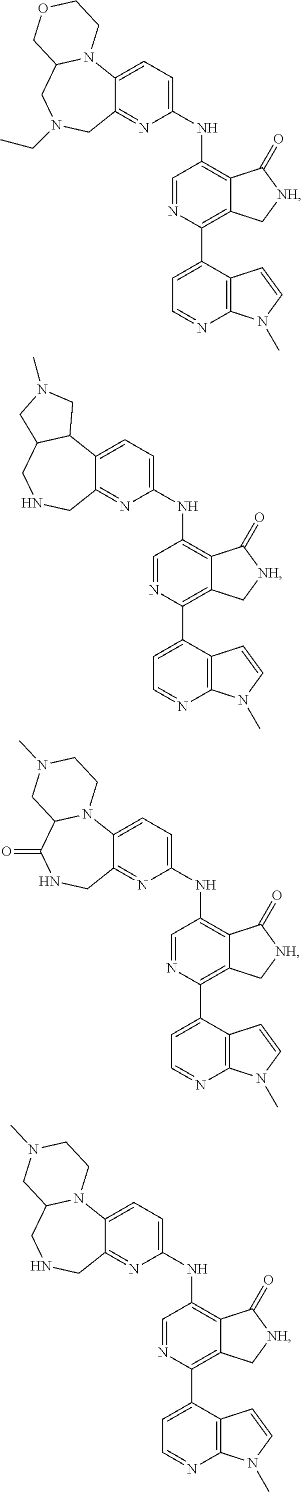 [SMILES: CCN1Cc2nc(Nc3cnc(-c4ccnc5c4ccn5C)c4c3C(=O)NC4)ccc2N2CCOCC2C1 ; CN1CC2CNCc3nc(Nc4cnc(-c5ccnc6c5ccn6C)c5c4C(=O)NC5)ccc3C2C1 ; CN1CCN2c3ccc(Nc4cnc(-c5ccnc6c5ccn6C)c5c4C(=O)NC5)nc3CNC(=O)C2C1 ; CN1CCN2c3ccc(Nc4cnc(-c5ccnc6c5ccn6C)c5c4C(=O)NC5)nc3CNCC2C1]

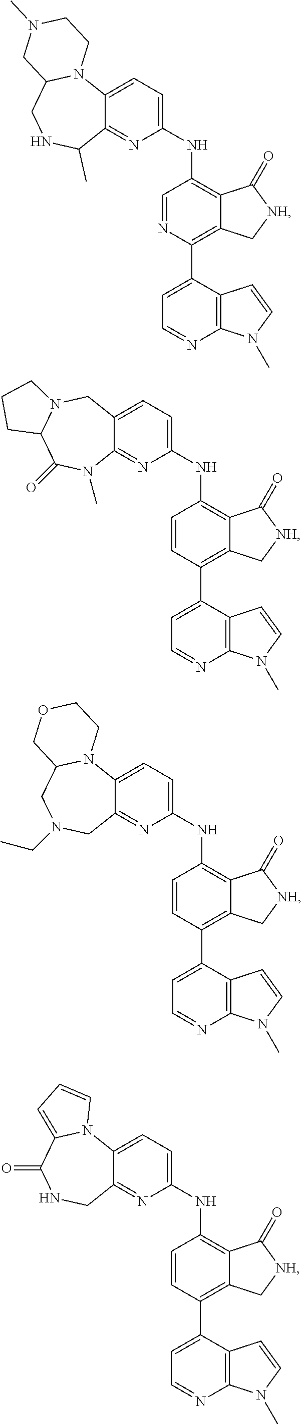 [SMILES: CC1NCC2CN(C)CCN2c2ccc(Nc3cnc(-c4ccnc5c4ccn5C)c4c3C(=O)NC4)nc21 ; CN1C(=O)C2CCCN2Cc2ccc(Nc3ccc(-c4ccnc5c4ccn5C)c4c3C(=O)NC4)nc21 ; CCN1Cc2nc(Nc3ccc(-c4ccnc5c4ccn5C)c4c3C(=O)NC4)ccc2N2CCOCC2C1 ; Cn1ccc2c(-c3ccc(Nc4ccc5c(n4)CNC(=O)c4cccn4-5)c4c3CNC4=O)ccnc21]

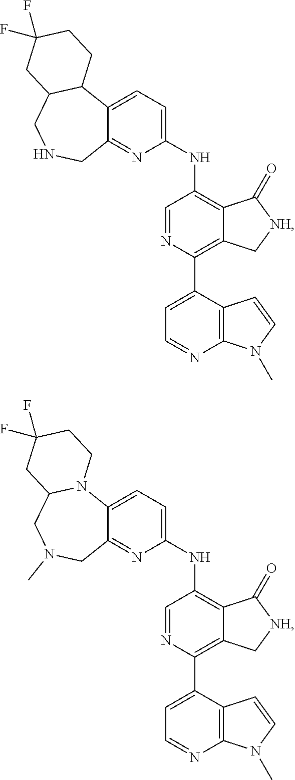 [SMILES: Cn1ccc2c(-c3ncc(Nc4ccc5c(n4)CNCC4CC(F)(F)CCC54)c4c3CNC4=O)ccnc21 ; CN1Cc2nc(Nc3cnc(-c4ccnc5c4ccn5C)c4c3C(=O)NC4)ccc2N2CCC(F)(F)CC2C1]

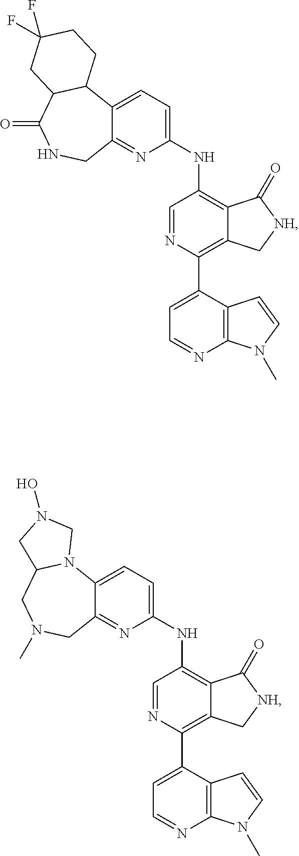 [SMILES: Cn1ccc2c(-c3ncc(Nc4ccc5c(n4)CNC(=O)C4CC(F)(F)CCC54)c4c3CNC4=O)ccnc21 ; CN1Cc2nc(Nc3cnc(-c4ccnc5c4ccn5C)c4c3C(=O)NC4)ccc2N2CN(O)CC2C1]

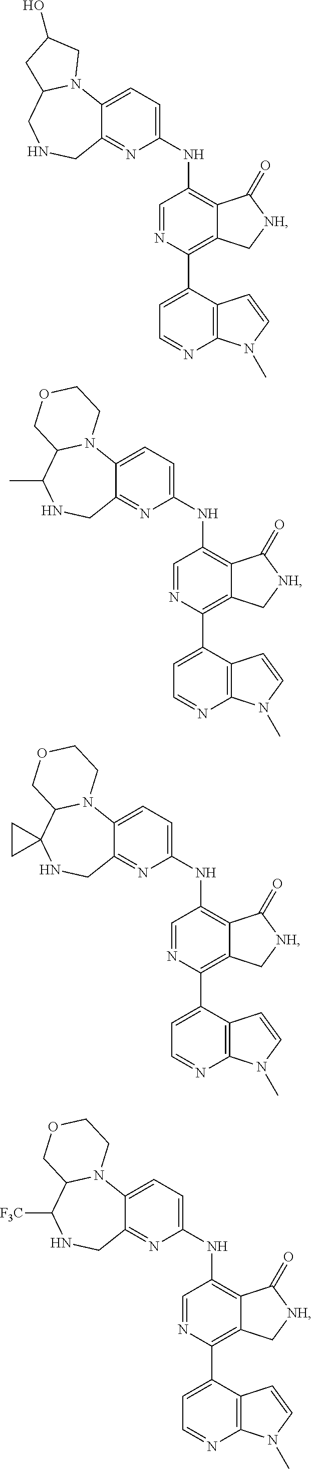 [SMILES: Cn1ccc2c(-c3ncc(Nc4ccc5c(n4)CNCC4CC(O)CN54)c4c3CNC4=O)ccnc21 ; CC1NCc2nc(Nc3cnc(-c4ccnc5c4ccn5C)c4c3C(=O)NC4)ccc2N2CCOCC12 ; Cn1ccc2c(-c3ncc(Nc4ccc5c(n4)CNC4(CC4)C4COCCN54)c4c3CNC4=O)ccnc21 ; Cn1ccc2c(-c3ncc(Nc4ccc5c(n4)CNC(C(F)(F)F)C4COCCN54)c4c3CNC4=O)ccnc21]

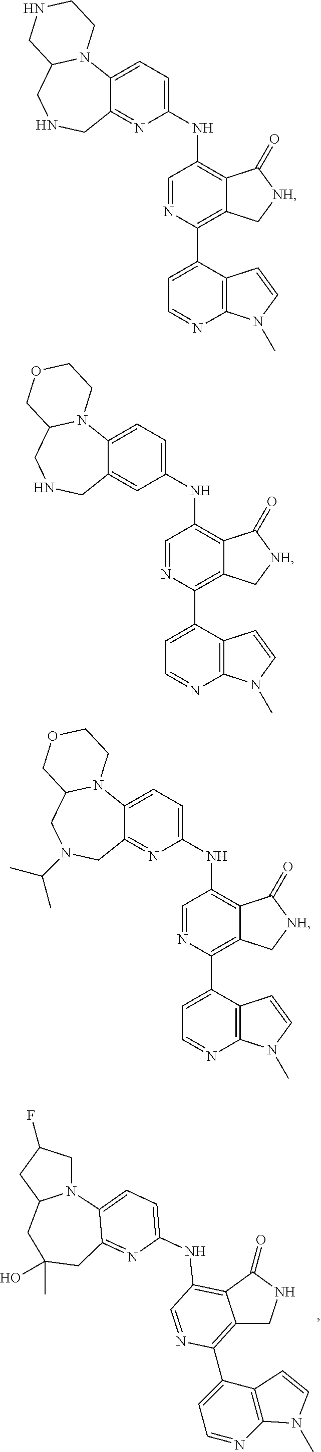 [SMILES: Cn1ccc2c(-c3ncc(Nc4ccc5c(n4)CNCC4CNCCN54)c4c3CNC4=O)ccnc21 ; Cn1ccc2c(-c3ncc(Nc4ccc5c(c4)CNCC4COCCN54)c4c3CNC4=O)ccnc21 ; CC(C)N1Cc2nc(Nc3cnc(-c4ccnc5c4ccn5C)c4c3C(=O)NC4)ccc2N2CCOCC2C1 ; Cn1ccc2c(-c3ncc(Nc4ccc5c(n4)CC(C)(O)CC4CC(F)CN54)c4c3CNC4=O)ccnc21]

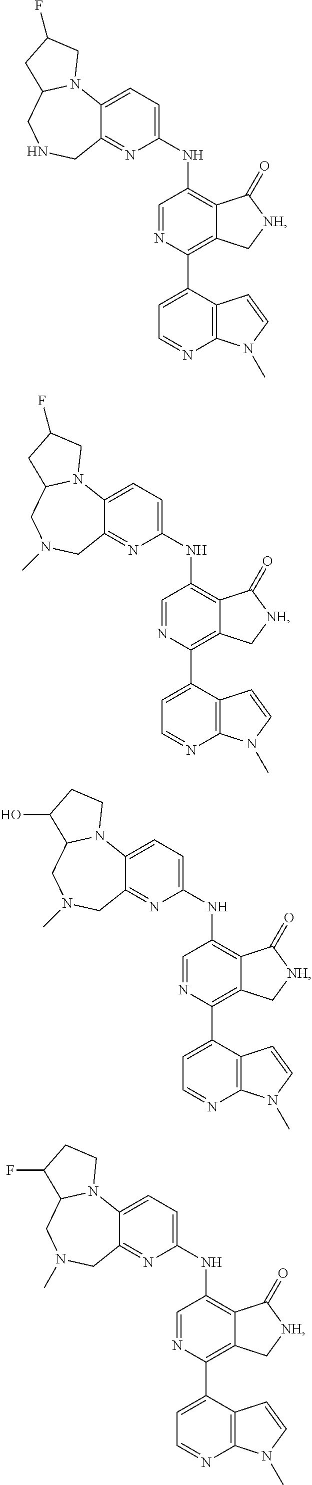 [SMILES: Cn1ccc2c(-c3ncc(Nc4ccc5c(n4)CNCC4CC(F)CN54)c4c3CNC4=O)ccnc21 ; CN1Cc2nc(Nc3cnc(-c4ccnc5c4ccn5C)c4c3C(=O)NC4)ccc2N2CC(F)CC2C1 ; CN1Cc2nc(Nc3cnc(-c4ccnc5c4ccn5C)c4c3C(=O)NC4)ccc2N2CCC(O)C2C1 ; CN1Cc2nc(Nc3cnc(-c4ccnc5c4ccn5C)c4c3C(=O)NC4)ccc2N2CCC(F)C2C1]

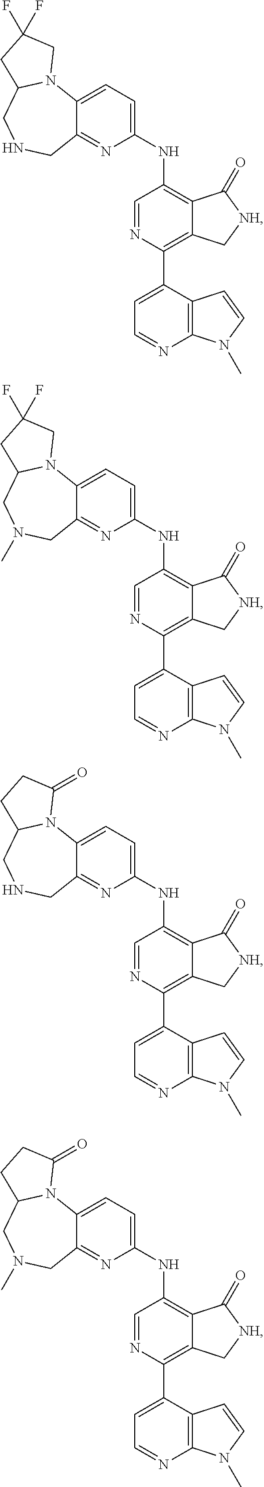 [SMILES: Cn1ccc2c(-c3ncc(Nc4ccc5c(n4)CNCC4CC(F)(F)CN54)c4c3CNC4=O)ccnc21 ; CN1Cc2nc(Nc3cnc(-c4ccnc5c4ccn5C)c4c3C(=O)NC4)ccc2N2CC(F)(F)CC2C1 ; Cn1ccc2c(-c3ncc(Nc4ccc5c(n4)CNCC4CCC(=O)N54)c4c3CNC4=O)ccnc21 ; CN1Cc2nc(Nc3cnc(-c4ccnc5c4ccn5C)c4c3C(=O)NC4)ccc2N2C(=O)CCC2C1]

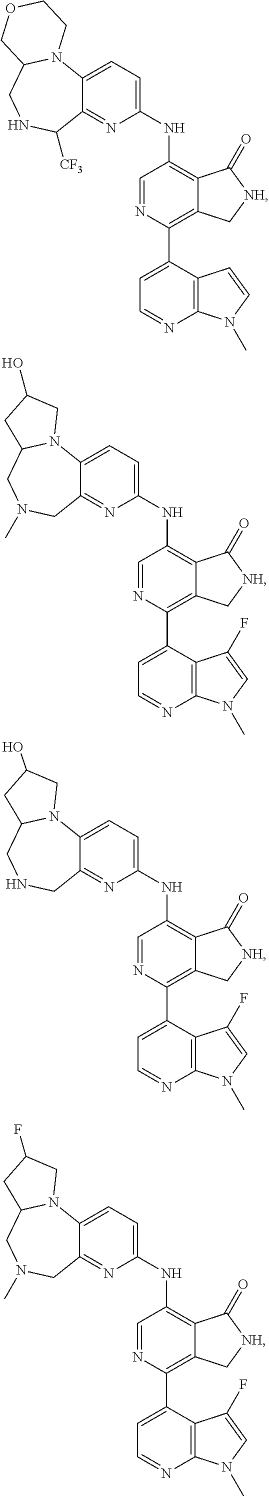 [SMILES: Cn1ccc2c(-c3ncc(Nc4ccc5c(n4)C(C(F)(F)F)NCC4COCCN54)c4c3CNC4=O)ccnc21 ; CN1Cc2nc(Nc3cnc(-c4ccnc5c4c(F)cn5C)c4c3C(=O)NC4)ccc2N2CC(O)CC2C1 ; Cn1cc(F)c2c(-c3ncc(Nc4ccc5c(n4)CNCC4CC(O)CN54)c4c3CNC4=O)ccnc21 ; CN1Cc2nc(Nc3cnc(-c4ccnc5c4c(F)cn5C)c4c3C(=O)NC4)ccc2N2CC(F)CC2C1]

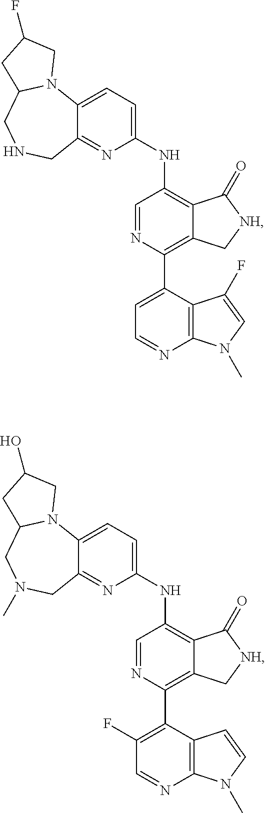 [SMILES: Cn1cc(F)c2c(-c3ncc(Nc4ccc5c(n4)CNCC4CC(F)CN54)c4c3CNC4=O)ccnc21 ; CN1Cc2nc(Nc3cnc(-c4c(F)cnc5c4ccn5C)c4c3C(=O)NC4)ccc2N2CC(O)CC2C1]

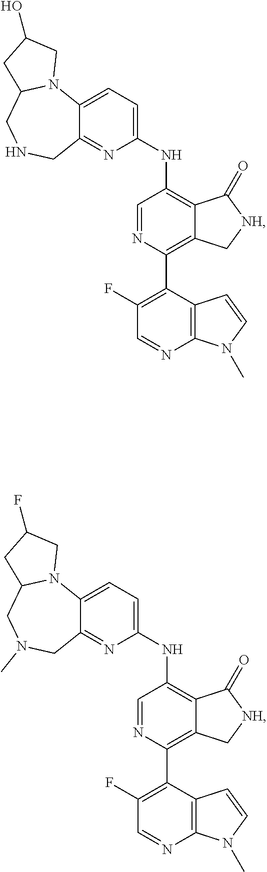 [SMILES: Cn1ccc2c(-c3ncc(Nc4ccc5c(n4)CNCC4CC(O)CN54)c4c3CNC4=O)c(F)cnc21 ; CN1Cc2nc(Nc3cnc(-c4c(F)cnc5c4ccn5C)c4c3C(=O)NC4)ccc2N2CC(F)CC2C1]

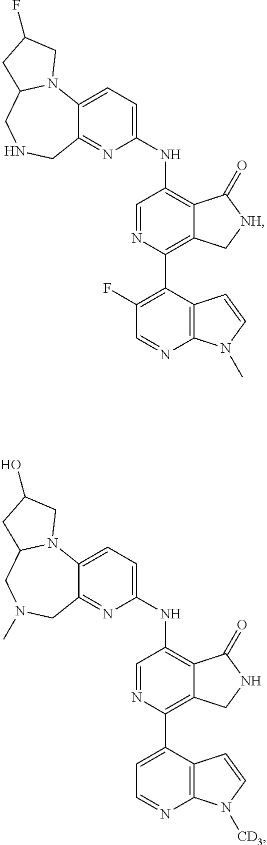 [SMILES: Cn1ccc2c(-c3ncc(Nc4ccc5c(n4)CNCC4CC(F)CN54)c4c3CNC4=O)c(F)cnc21 ; [2H]C([2H])([2H])n1ccc2c(-c3ncc(Nc4ccc5c(n4)CN(C)CC4CC(O)CN54)c4c3CNC4=O)ccnc21]

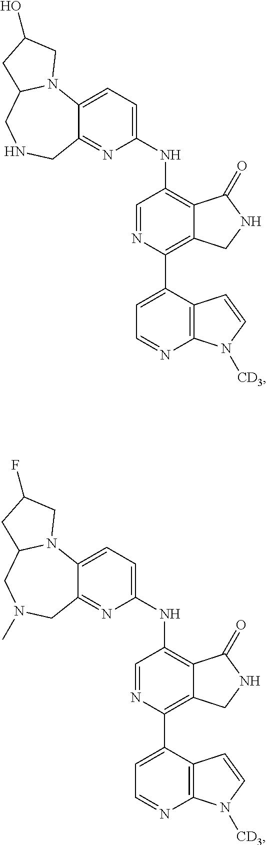 [SMILES: [2H]C([2H])([2H])n1ccc2c(-c3ncc(Nc4ccc5c(n4)CNCC4CC(O)CN54)c4c3CNC4=O)ccnc21 ; [2H]C([2H])([2H])n1ccc2c(-c3ncc(Nc4ccc5c(n4)CN(C)CC4CC(F)CN54)c4c3CNC4=O)ccnc21]

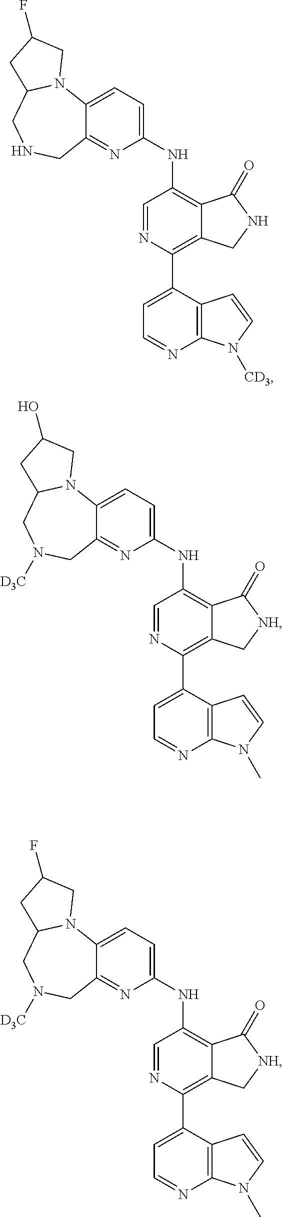 [SMILES: [2H]C([2H])([2H])n1ccc2c(-c3ncc(Nc4ccc5c(n4)CNCC4CC(F)CN54)c4c3CNC4=O)ccnc21 ; [2H]C([2H])([2H])N1Cc2nc(Nc3cnc(-c4ccnc5c4ccn5C)c4c3C(=O)NC4)ccc2N2CC(O)CC2C1 ; [2H]C([2H])([2H])N1Cc2nc(Nc3cnc(-c4ccnc5c4ccn5C)c4c3C(=O)NC4)ccc2N2CC(F)CC2C1]

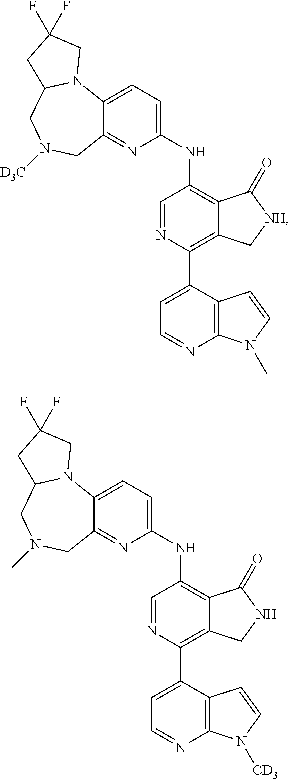 [SMILES: [2H]C([2H])([2H])N1Cc2nc(Nc3cnc(-c4ccnc5c4ccn5C)c4c3C(=O)NC4)ccc2N2CC(F)(F)CC2C1 ; [2H]C([2H])([2H])n1ccc2c(-c3ncc(Nc4ccc5c(n4)CN(C)CC4CC(F)(F)CN54)c4c3CNC4=O)ccnc21]

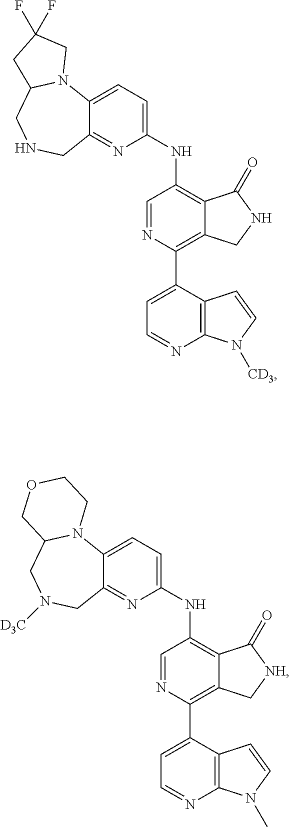 [SMILES: [2H]C([2H])([2H])n1ccc2c(-c3ncc(Nc4ccc5c(n4)CNCC4CC(F)(F)CN54)c4c3CNC4=O)ccnc21 ; [2H]C([2H])([2H])N1Cc2nc(Nc3cnc(-c4ccnc5c4ccn5C)c4c3C(=O)NC4)ccc2N2CCOCC2C1]

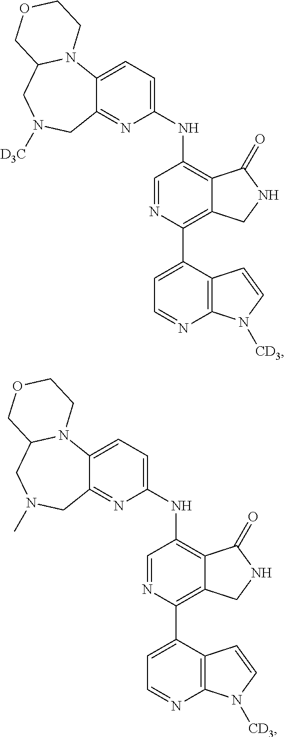 [SMILES: [2H]C([2H])([2H])N1Cc2nc(Nc3cnc(-c4ccnc5c4ccn5C([2H])([2H])[2H])c4c3C(=O)NC4)ccc2N2CCOCC2C1 ; [2H]C([2H])([2H])n1ccc2c(-c3ncc(Nc4ccc5c(n4)CN(C)CC4COCCN54)c4c3CNC4=O)ccnc21]

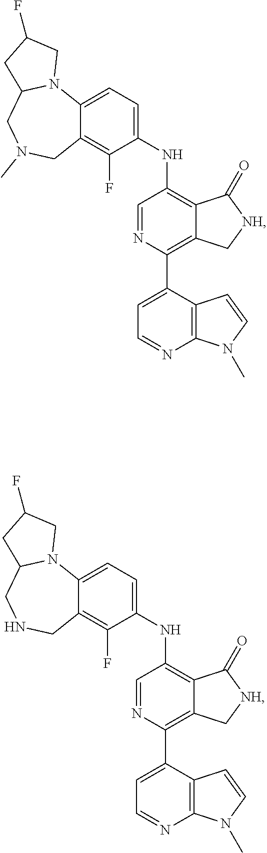 [SMILES: CN1Cc2c(ccc(Nc3cnc(-c4ccnc5c4ccn5C)c4c3C(=O)NC4)c2F)N2CC(F)CC2C1 ; Cn1ccc2c(-c3ncc(Nc4ccc5c(c4F)CNCC4CC(F)CN54)c4c3CNC4=O)ccnc21]

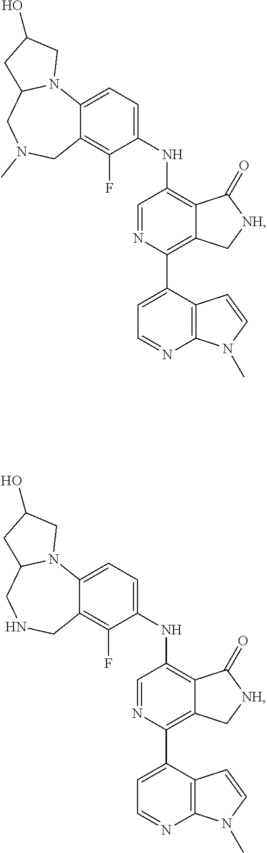 [SMILES: CN1Cc2c(ccc(Nc3cnc(-c4ccnc5c4ccn5C)c4c3C(=O)NC4)c2F)N2CC(O)CC2C1 ; Cn1ccc2c(-c3ncc(Nc4ccc5c(c4F)CNCC4CC(O)CN54)c4c3CNC4=O)ccnc21]

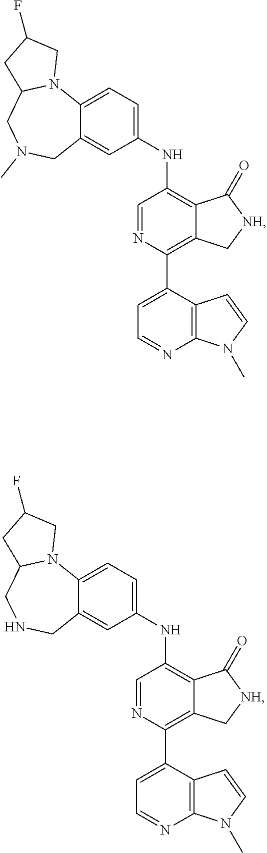 [SMILES: CN1Cc2cc(Nc3cnc(-c4ccnc5c4ccn5C)c4c3C(=O)NC4)ccc2N2CC(F)CC2C1 ; Cn1ccc2c(-c3ncc(Nc4ccc5c(c4)CNCC4CC(F)CN54)c4c3CNC4=O)ccnc21]

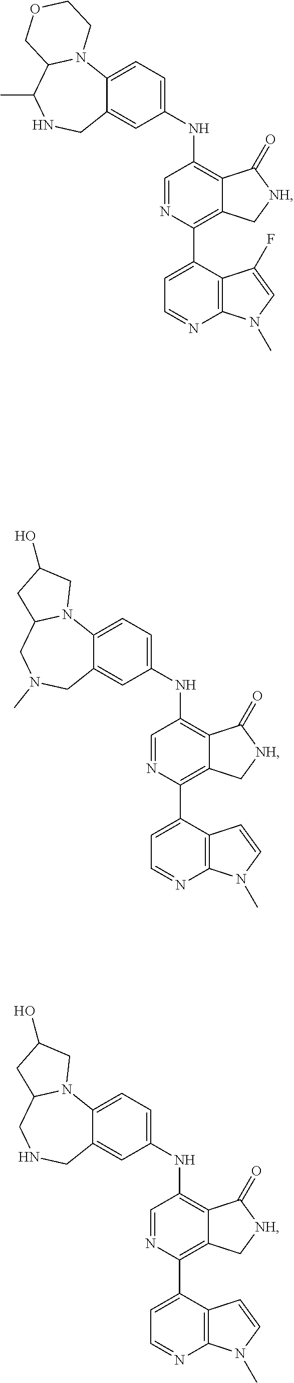 [SMILES: CC1NCc2cc(Nc3cnc(-c4ccnc5c4c(F)cn5C)c4c3C(=O)NC4)ccc2N2CCOCC12 ; CN1Cc2cc(Nc3cnc(-c4ccnc5c4ccn5C)c4c3C(=O)NC4)ccc2N2CC(O)CC2C1 ; Cn1ccc2c(-c3ncc(Nc4ccc5c(c4)CNCC4CC(O)CN54)c4c3CNC4=O)ccnc21]

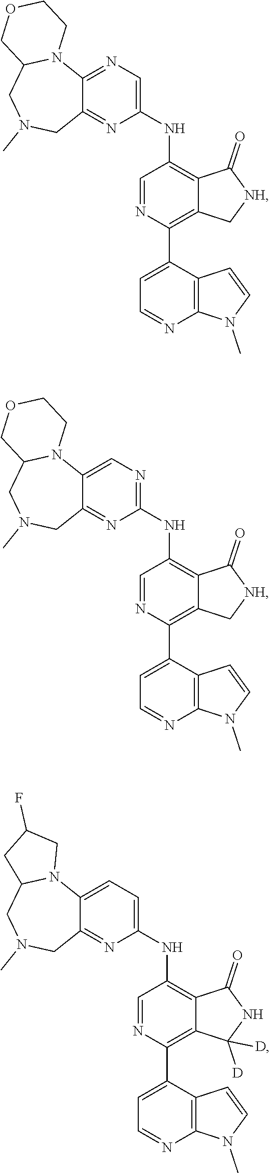

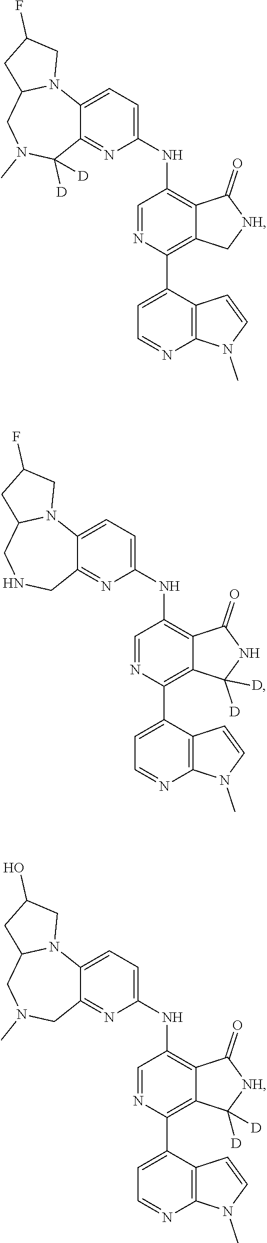

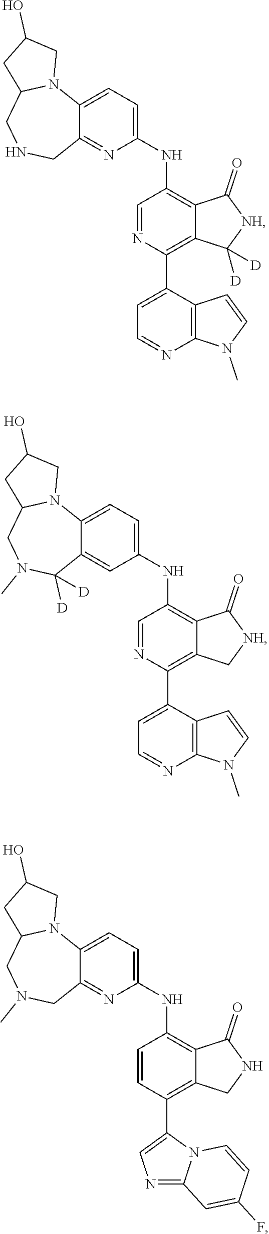 [SMILES: [2H]C1([2H])NC(=O)c2c(Nc3ccc4c(n3)CNCC3CC(O)CN43)cnc(-c3ccnc4c3ccn4C)c21 ; [2H]C1([2H])c2cc(Nc3cnc(-c4ccnc5c4ccn5C)c4c3C(=O)NC4)ccc2N2CC(O)CC2CN1C ; CN1Cc2nc(Nc3ccc(-c4cnc5cc(F)ccn45)c4c3C(=O)NC4)ccc2N2CC(O)CC2C1]

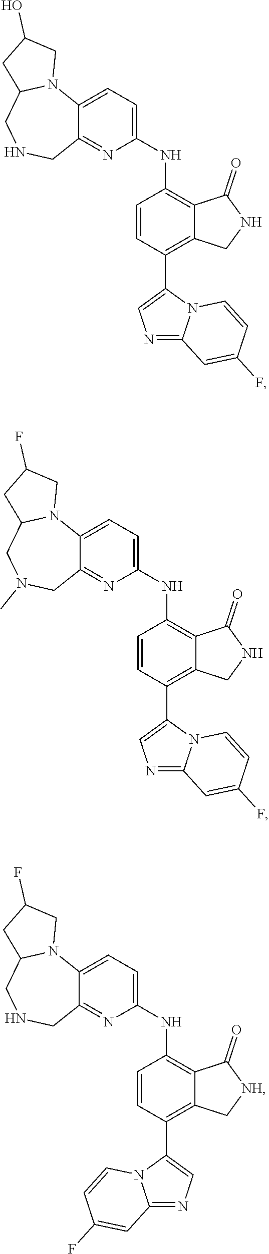 [SMILES: O=C1NCc2c(-c3cnc4cc(F)ccn34)ccc(Nc3ccc4c(n3)CNCC3CC(O)CN43)c21 ; CN1Cc2nc(Nc3ccc(-c4cnc5cc(F)ccn45)c4c3C(=O)NC4)ccc2N2CC(F)CC2C1 ; O=C1NCc2c(-c3cnc4cc(F)ccn34)ccc(Nc3ccc4c(n3)CNCC3CC(F)CN43)c21]

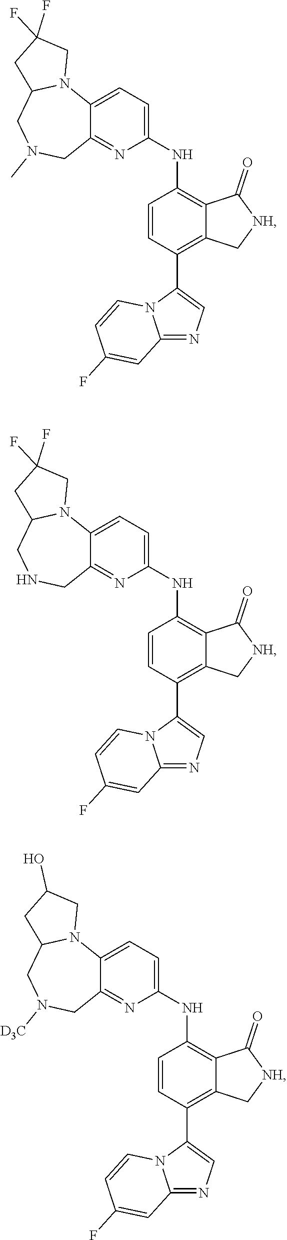 [SMILES: CN1Cc2nc(Nc3ccc(-c4cnc5cc(F)ccn45)c4c3C(=O)NC4)ccc2N2CC(F)(F)CC2C1 ; O=C1NCc2c(-c3cnc4cc(F)ccn34)ccc(Nc3ccc4c(n3)CNCC3CC(F)(F)CN43)c21 ; [2H]C([2H])([2H])N1Cc2nc(Nc3ccc(-c4cnc5cc(F)ccn45)c4c3C(=O)NC4)ccc2N2CC(O)CC2C1]

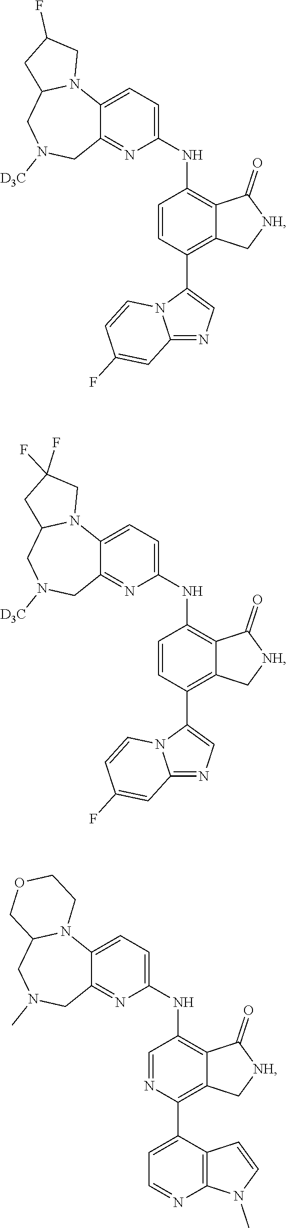 [SMILES: [2H]C([2H])([2H])N1Cc2nc(Nc3ccc(-c4cnc5cc(F)ccn45)c4c3C(=O)NC4)ccc2N2CC(F)CC2C1 ; [2H]C([2H])([2H])N1Cc2nc(Nc3ccc(-c4cnc5cc(F)ccn45)c4c3C(=O)NC4)ccc2N2CC(F)(F)CC2C1 ; CN1Cc2nc(Nc3cnc(-c4ccnc5c4ccn5C)c4c3C(=O)NC4)ccc2N2CCOCC2C1]

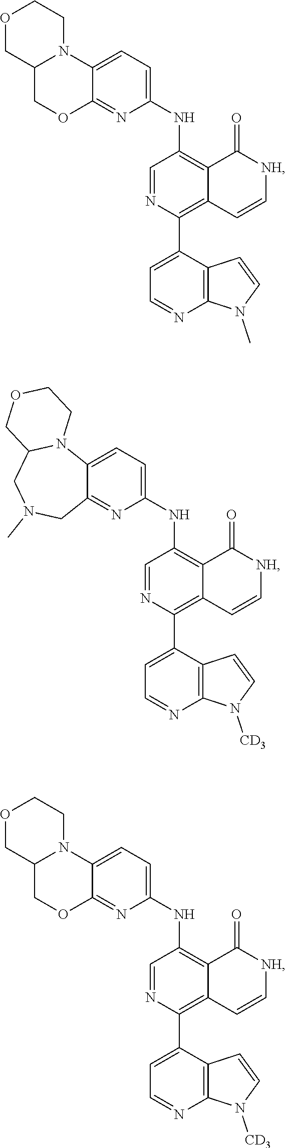 [SMILES: Cn1ccc2c(-c3ncc(Nc4ccc5c(n4)OCC4COCCN54)c4c(=O)[nH]ccc34)ccnc21 ; [2H]C([2H])([2H])n1ccc2c(-c3ncc(Nc4ccc5c(n4)CN(C)CC4COCCN54)c4c(=O)[nH]ccc34)ccnc21 ; [2H]C([2H])([2H])n1ccc2c(-c3ncc(Nc4ccc5c(n4)OCC4COCCN54)c4c(=O)[nH]ccc34)ccnc21]

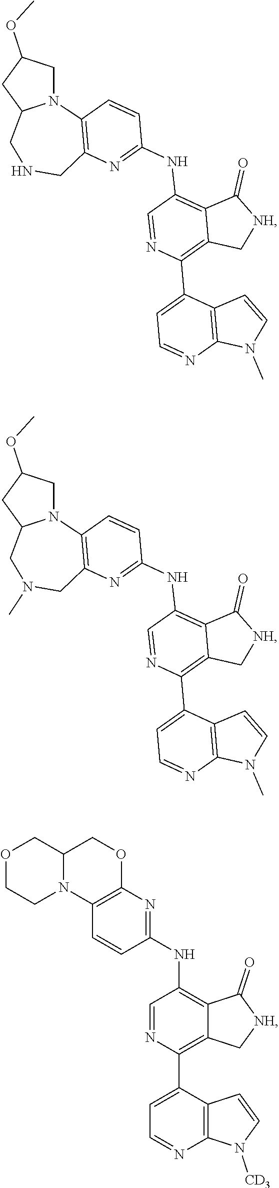 [SMILES: COC1CC2CNCc3nc(Nc4cnc(-c5ccnc6c5ccn6C)c5c4C(=O)NC5)ccc3N2C1 ; COC1CC2CN(C)Cc3nc(Nc4cnc(-c5ccnc6c5ccn6C)c5c4C(=O)NC5)ccc3N2C1 ; [2H]C([2H])([2H])n1ccc2c(-c3ncc(Nc4ccc5c(n4)OCC4COCCN54)c4c3CNC4=O)ccnc21]

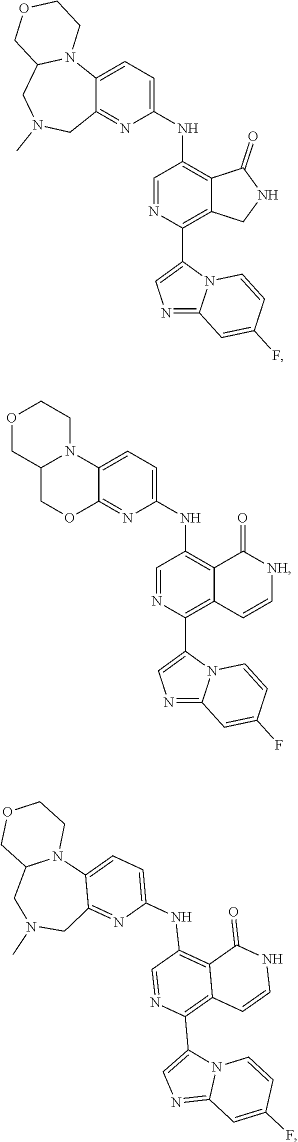 [SMILES: CN1Cc2nc(Nc3cnc(-c4cnc5cc(F)ccn45)c4c3C(=O)NC4)ccc2N2CCOCC2C1 ; O=c1[nH]ccc2c(-c3cnc4cc(F)ccn34)ncc(Nc3ccc4c(n3)OCC3COCCN43)c12 ; CN1Cc2nc(Nc3cnc(-c4cnc5cc(F)ccn45)c4cc[nH]c(=O)c34)ccc2N2CCOCC2C1]

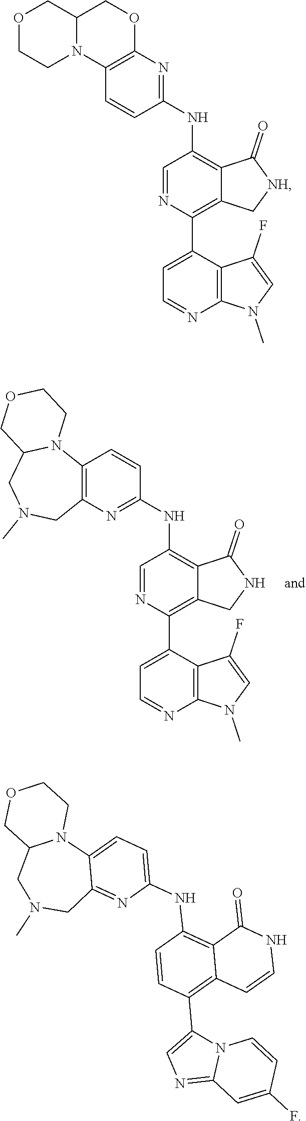 [SMILES: Cn1cc(F)c2c(-c3ncc(Nc4ccc5c(n4)OCC4COCCN54)c4c3CNC4=O)ccnc21 ; CN1Cc2nc(Nc3cnc(-c4ccnc5c4c(F)cn5C)c4c3C(=O)NC4)ccc2N2CCOCC2C1 ; CN1Cc2nc(Nc3ccc(-c4cnc5cc(F)ccn45)c4cc[nH]c(=O)c34)ccc2N2CCOCC2C1]

In some embodiments of the present disclosure, disclosed herein is the above compound, a stereoisomer or a pharmaceutically acceptable salt thereof, which is selected from:

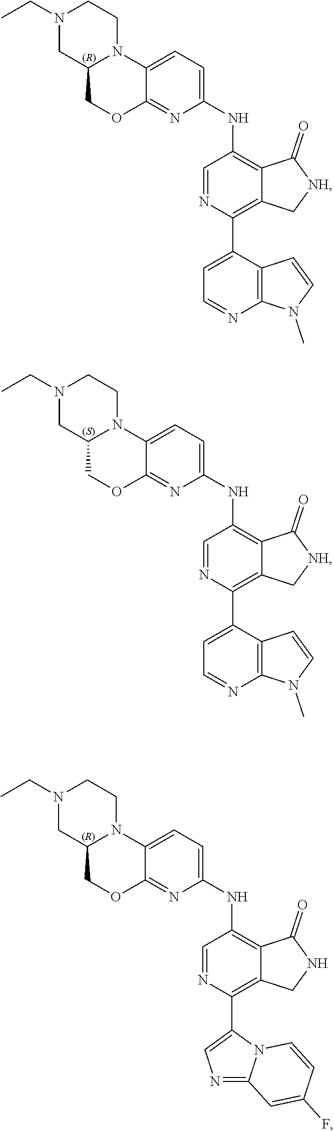

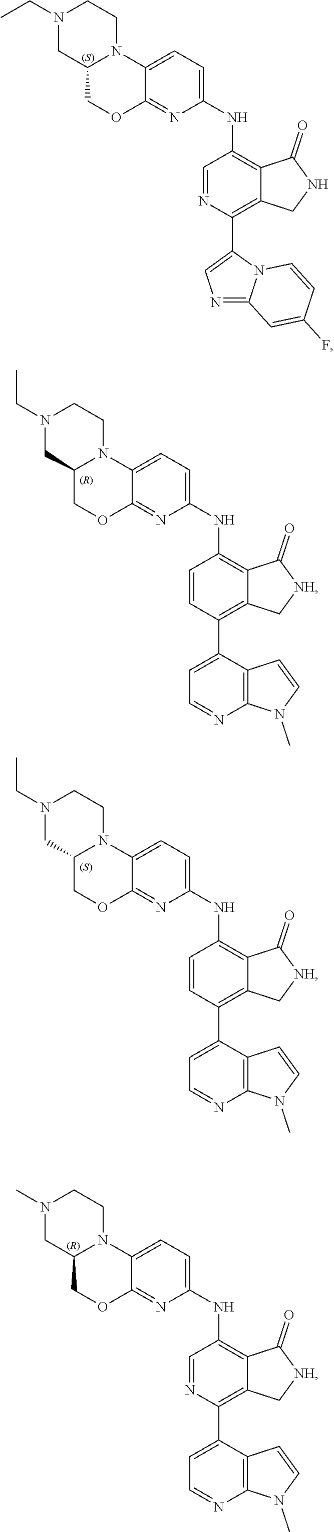

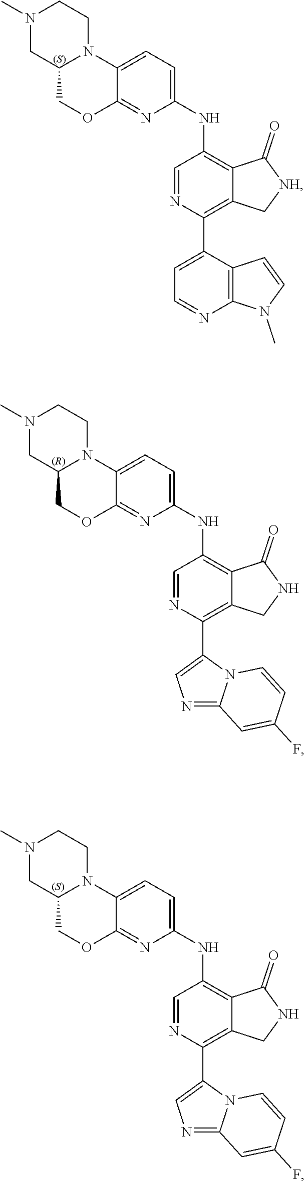

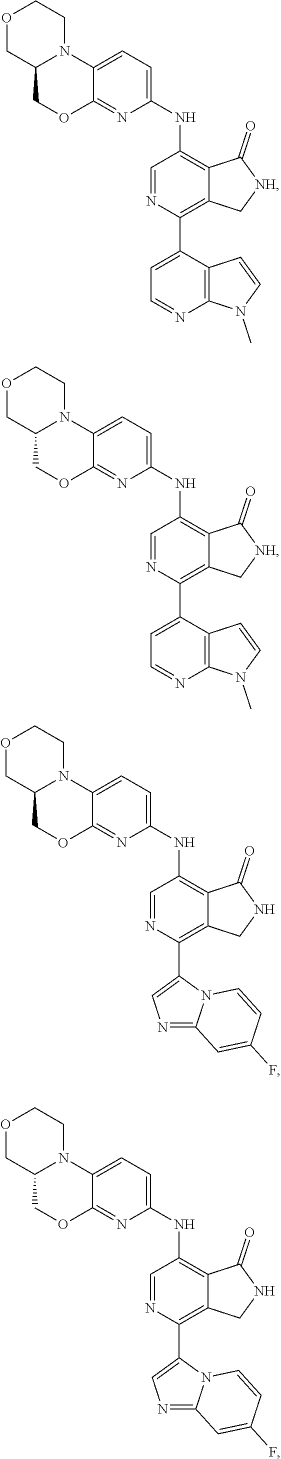 [SMILES: Cn1ccc2c(-c3ncc(Nc4ccc5c(n4)OC[C@H]4COCCN54)c4c3CNC4=O)ccnc21 ; Cn1ccc2c(-c3ncc(Nc4ccc5c(n4)OC[C@@H]4COCCN54)c4c3CNC4=O)ccnc21 ; O=C1NCc2c(-c3cnc4cc(F)ccn34)ncc(Nc3ccc4c(n3)OC[C@H]3COCCN43)c21 ; O=C1NCc2c(-c3cnc4cc(F)ccn34)ncc(Nc3ccc4c(n3)OC[C@@H]3COCCN43)c21]

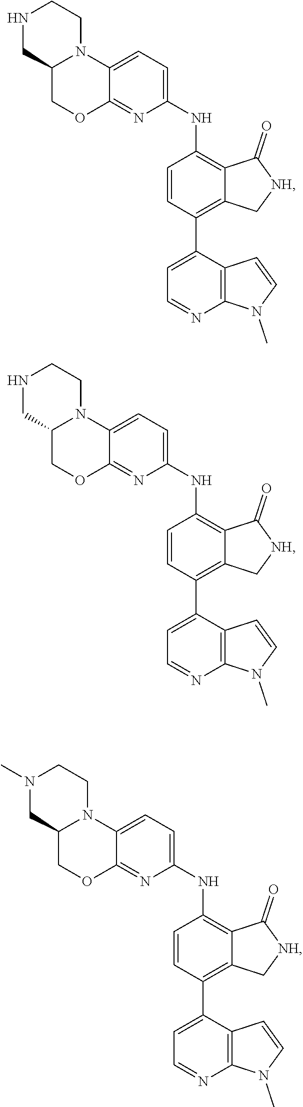 [SMILES: Cn1ccc2c(-c3ccc(Nc4ccc5c(n4)OC[C@H]4CNCCN54)c4c3CNC4=O)ccnc21 ; Cn1ccc2c(-c3ccc(Nc4ccc5c(n4)OC[C@@H]4CNCCN54)c4c3CNC4=O)ccnc21 ; CN1CCN2c3ccc(Nc4ccc(-c5ccnc6c5ccn6C)c5c4C(=O)NC5)nc3OC[C@H]2C1]

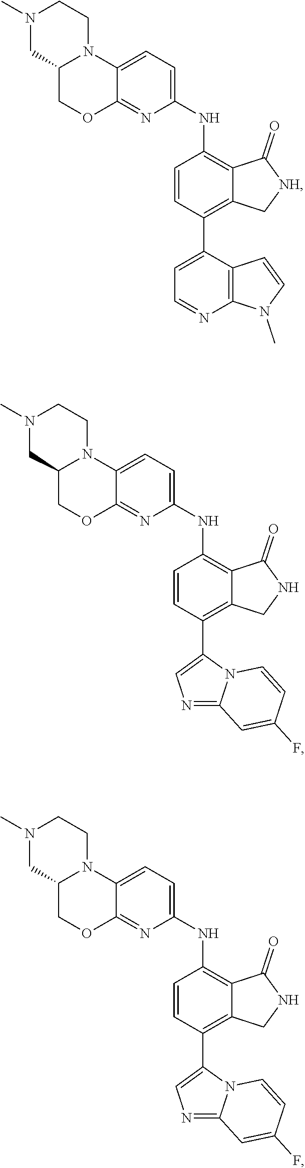 [SMILES: CN1CCN2c3ccc(Nc4ccc(-c5ccnc6c5ccn6C)c5c4C(=O)NC5)nc3OC[C@@H]2C1 ; CN1CCN2c3ccc(Nc4ccc(-c5cnc6cc(F)ccn56)c5c4C(=O)NC5)nc3OC[C@H]2C1 ; CN1CCN2c3ccc(Nc4ccc(-c5cnc6cc(F)ccn56)c5c4C(=O)NC5)nc3OC[C@@H]2C1]

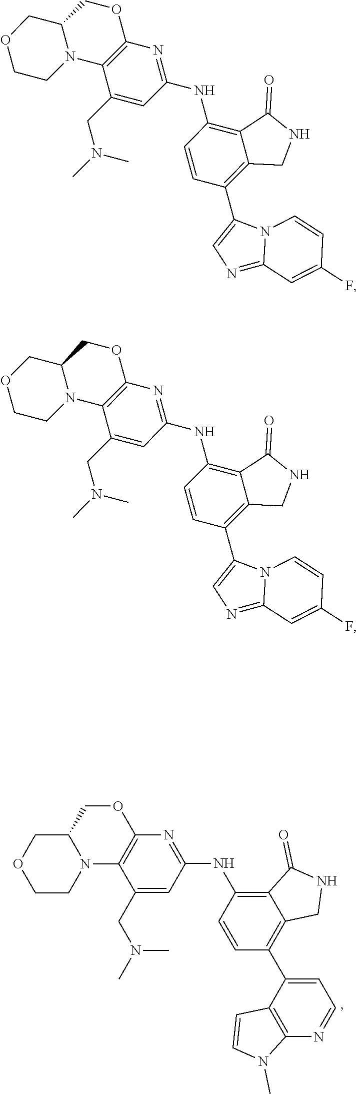 [SMILES: CN(C)Cc1cc(Nc2ccc(-c3cnc4cc(F)ccn34)c3c2C(=O)NC3)nc2c1N1CCOC[C@@H]1CO2 ; CN(C)Cc1cc(Nc2ccc(-c3cnc4cc(F)ccn34)c3c2C(=O)NC3)nc2c1N1CCOC[C@H]1CO2 ; CN(C)Cc1cc(Nc2ccc(-c3ccnc4c3ccn4C)c3c2C(=O)NC3)nc2c1N1CCOC[C@@H]1CO2]

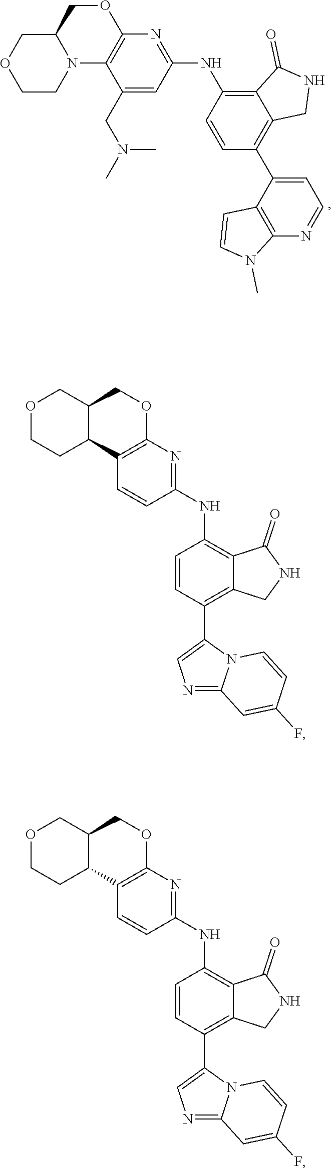 [SMILES: CN(C)Cc1cc(Nc2ccc(-c3ccnc4c3ccn4C)c3c2C(=O)NC3)nc2c1N1CCOC[C@H]1CO2 ; O=C1NCc2c(-c3cnc4cc(F)ccn34)ccc(Nc3ccc4c(n3)OC[C@@H]3COCC[C@H]43)c21 ; O=C1NCc2c(-c3cnc4cc(F)ccn34)ccc(Nc3ccc4c(n3)OC[C@@H]3COCC[C@@H]43)c21]

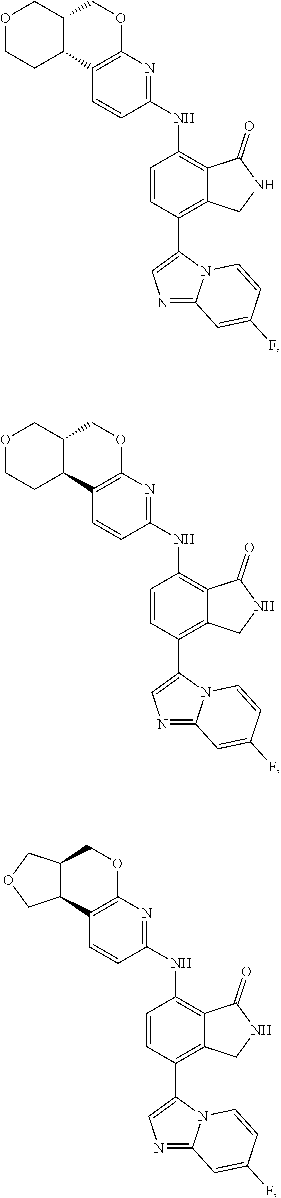 [SMILES: O=C1NCc2c(-c3cnc4cc(F)ccn34)ccc(Nc3ccc4c(n3)OC[C@H]3COCC[C@@H]43)c21 ; O=C1NCc2c(-c3cnc4cc(F)ccn34)ccc(Nc3ccc4c(n3)OC[C@H]3COCC[C@H]43)c21 ; O=C1NCc2c(-c3cnc4cc(F)ccn34)ccc(Nc3ccc4c(n3)OC[C@@H]3COC[C@H]43)c21]

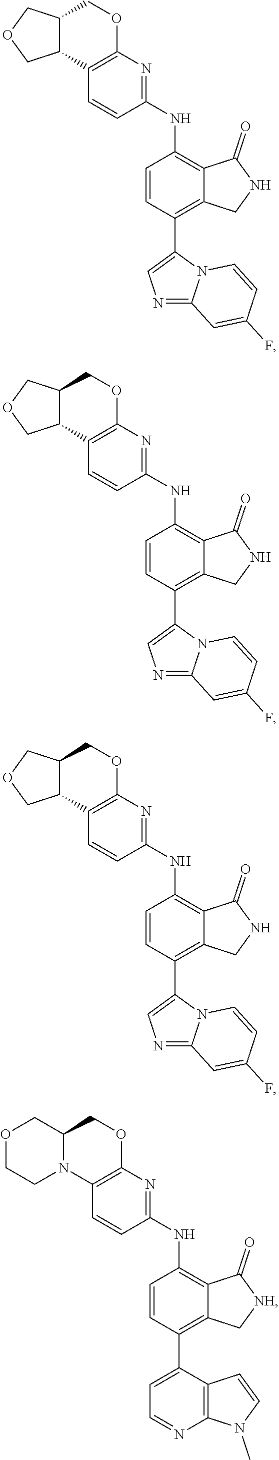 [SMILES: O=C1NCc2c(-c3cnc4cc(F)ccn34)ccc(Nc3ccc4c(n3)OC[C@H]3COC[C@@H]43)c21 ; O=C1NCc2c(-c3cnc4cc(F)ccn34)ccc(Nc3ccc4c(n3)OC[C@@H]3COC[C@@H]43)c21 ; O=C1NCc2c(-c3cnc4cc(F)ccn34)ccc(Nc3ccc4c(n3)OC[C@@H]3COC[C@@H]43)c21 ; Cn1ccc2c(-c3ccc(Nc4ccc5c(n4)OC[C@@H]4COCCN54)c4c3CNC4=O)ccnc21]

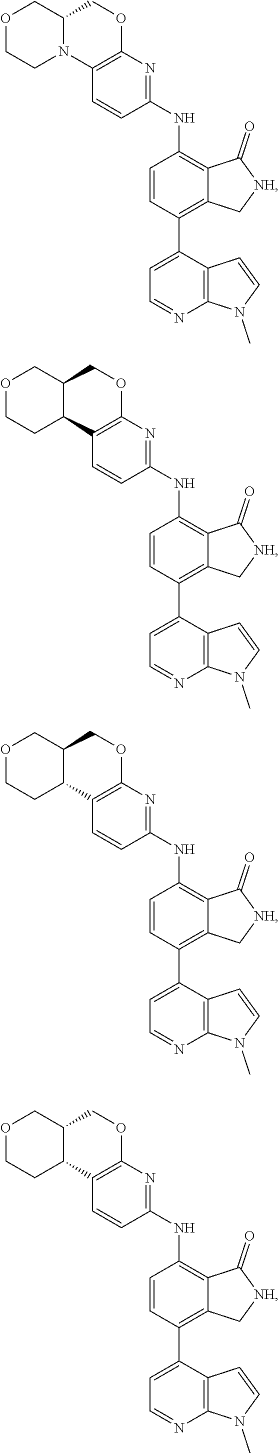 [SMILES: Cn1ccc2c(-c3ccc(Nc4ccc5c(n4)OC[C@H]4COCCN54)c4c3CNC4=O)ccnc21 ; Cn1ccc2c(-c3ccc(Nc4ccc5c(n4)OC[C@@H]4COCC[C@H]54)c4c3CNC4=O)ccnc21 ; Cn1ccc2c(-c3ccc(Nc4ccc5c(n4)OC[C@@H]4COCC[C@@H]54)c4c3CNC4=O)ccnc21 ; Cn1ccc2c(-c3ccc(Nc4ccc5c(n4)OC[C@H]4COCC[C@@H]54)c4c3CNC4=O)ccnc21]

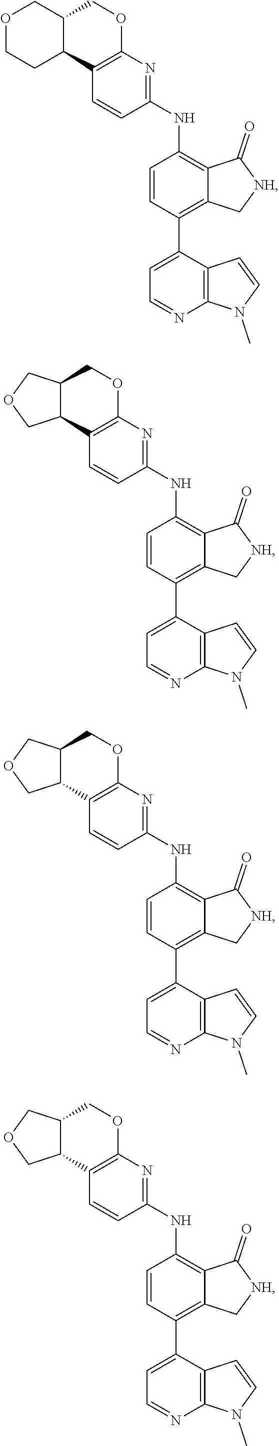 [SMILES: Cn1ccc2c(-c3ccc(Nc4ccc5c(n4)OC[C@H]4COCC[C@H]54)c4c3CNC4=O)ccnc21 ; Cn1ccc2c(-c3ccc(Nc4ccc5c(n4)OC[C@@H]4COC[C@H]54)c4c3CNC4=O)ccnc21 ; Cn1ccc2c(-c3ccc(Nc4ccc5c(n4)OC[C@@H]4COC[C@@H]54)c4c3CNC4=O)ccnc21 ; Cn1ccc2c(-c3ccc(Nc4ccc5c(n4)OC[C@H]4COC[C@@H]54)c4c3CNC4=O)ccnc21]

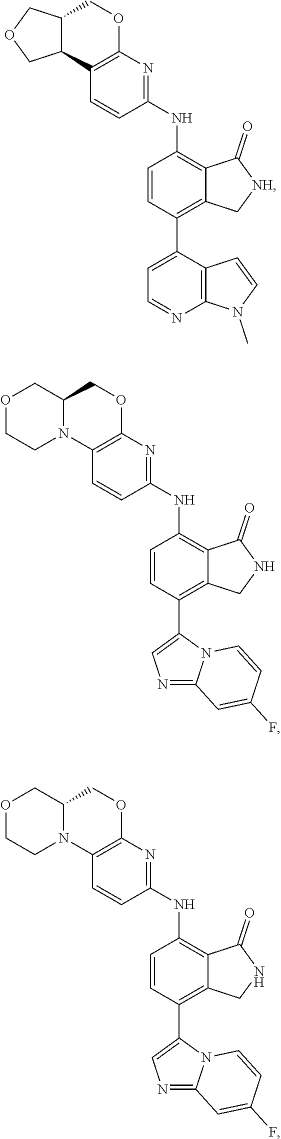 [SMILES: Cn1ccc2c(-c3ccc(Nc4ccc5c(n4)OC[C@H]4COC[C@H]54)c4c3CNC4=O)ccnc21 ; O=C1NCc2c(-c3cnc4cc(F)ccn34)ccc(Nc3ccc4c(n3)OC[C@@H]3COCCN43)c21 ; O=C1NCc2c(-c3cnc4cc(F)ccn34)ccc(Nc3ccc4c(n3)OC[C@H]3COCCN43)c21]

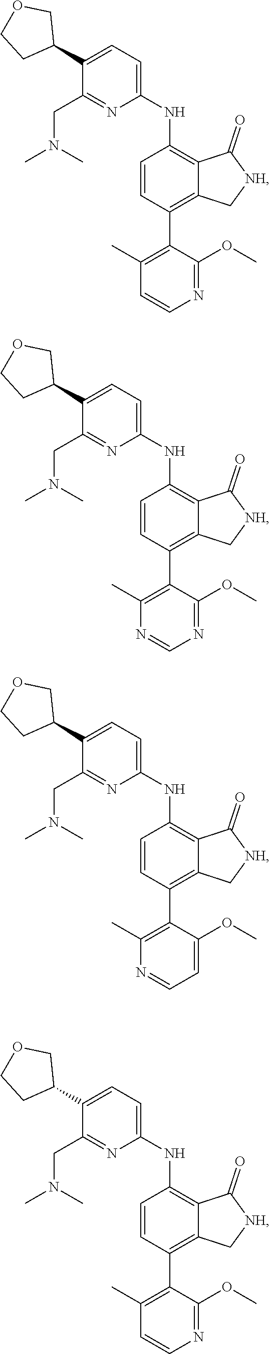 [SMILES: COc1nccc(C)c1-c1ccc(Nc2ccc([C@H]3CCOC3)c(CN(C)C)n2)c2c1CNC2=O ; COc1ncnc(C)c1-c1ccc(Nc2ccc([C@H]3CCOC3)c(CN(C)C)n2)c2c1CNC2=O ; COc1ccnc(C)c1-c1ccc(Nc2ccc([C@H]3CCOC3)c(CN(C)C)n2)c2c1CNC2=O ; COc1nccc(C)c1-c1ccc(Nc2ccc([C@@H]3CCOC3)c(CN(C)C)n2)c2c1CNC2=O]

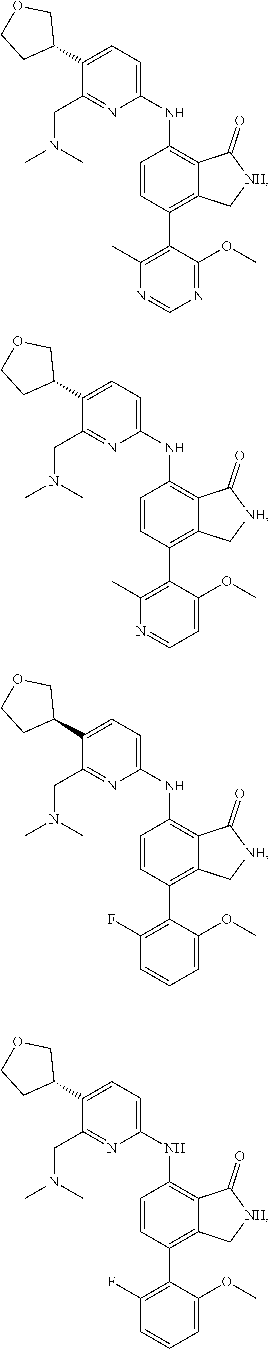 [SMILES: COc1ncnc(C)c1-c1ccc(Nc2ccc([C@@H]3CCOC3)c(CN(C)C)n2)c2c1CNC2=O ; COc1ccnc(C)c1-c1ccc(Nc2ccc([C@@H]3CCOC3)c(CN(C)C)n2)c2c1CNC2=O ; COc1cccc(F)c1-c1ccc(Nc2ccc([C@H]3CCOC3)c(CN(C)C)n2)c2c1CNC2=O ; COc1cccc(F)c1-c1ccc(Nc2ccc([C@@H]3CCOC3)c(CN(C)C)n2)c2c1CNC2=O]

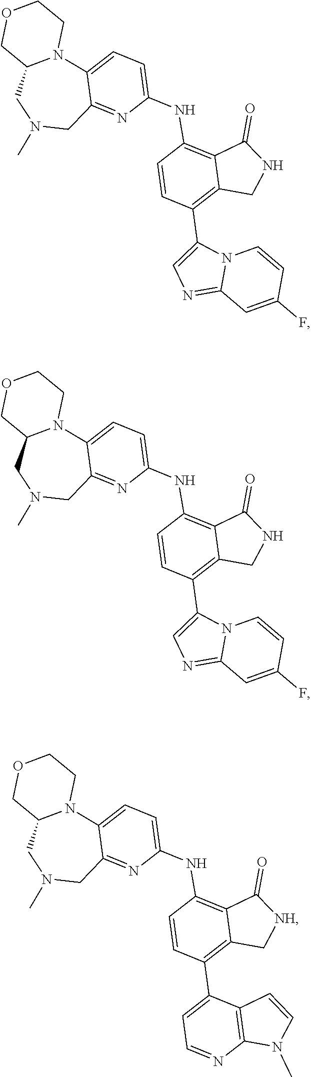 [SMILES: CN1Cc2nc(Nc3ccc(-c4cnc5cc(F)ccn45)c4c3C(=O)NC4)ccc2N2CCOC[C@H]2C1 ; CN1Cc2nc(Nc3ccc(-c4cnc5cc(F)ccn45)c4c3C(=O)NC4)ccc2N2CCOC[C@@H]2C1 ; CN1Cc2nc(Nc3ccc(-c4ccnc5c4ccn5C)c4c3C(=O)NC4)ccc2N2CCOC[C@H]2C1]

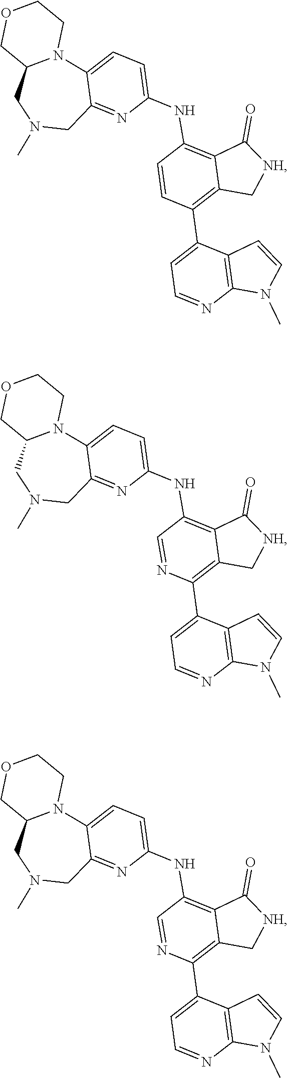 [SMILES: CN1Cc2nc(Nc3ccc(-c4ccnc5c4ccn5C)c4c3C(=O)NC4)ccc2N2CCOC[C@@H]2C1 ; CN1Cc2nc(Nc3cnc(-c4ccnc5c4ccn5C)c4c3C(=O)NC4)ccc2N2CCOC[C@H]2C1 ; CN1Cc2nc(Nc3cnc(-c4ccnc5c4ccn5C)c4c3C(=O)NC4)ccc2N2CCOC[C@@H]2C1]

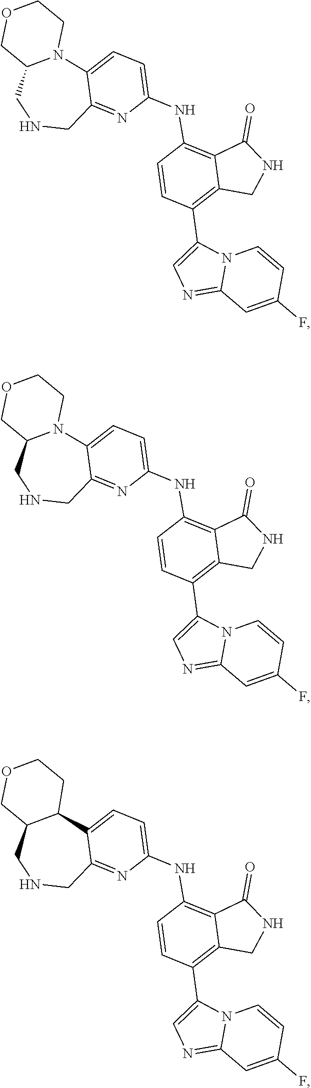 [SMILES: O=C1NCc2c(-c3cnc4cc(F)ccn34)ccc(Nc3ccc4c(n3)CNC[C@@H]3COCCN43)c21 ; O=C1NCc2c(-c3cnc4cc(F)ccn34)ccc(Nc3ccc4c(n3)CNC[C@H]3COCCN43)c21 ; O=C1NCc2c(-c3cnc4cc(F)ccn34)ccc(Nc3ccc4c(n3)CNC[C@H]3COCC[C@@H]43)c21]

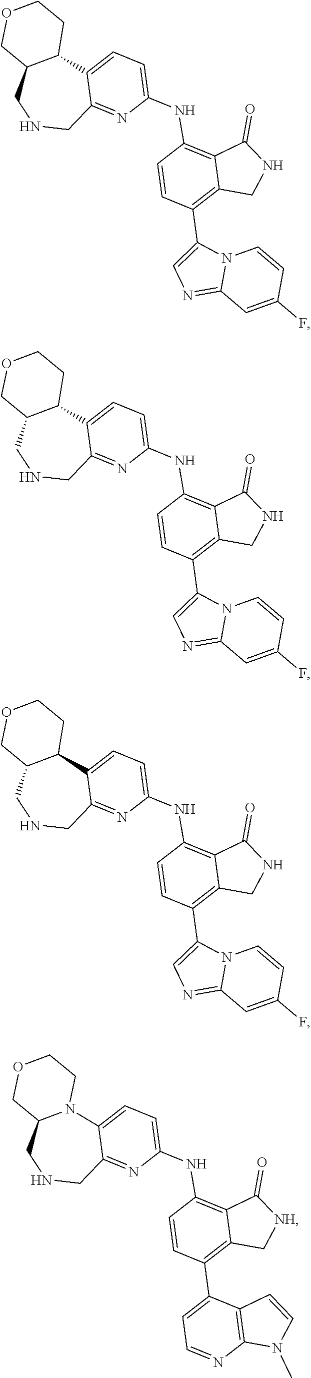 [SMILES: O=C1NCc2c(-c3cnc4cc(F)ccn34)ccc(Nc3ccc4c(n3)CNC[C@H]3COCC[C@H]43)c21 ; O=C1NCc2c(-c3cnc4cc(F)ccn34)ccc(Nc3ccc4c(n3)CNC[C@@H]3COCC[C@H]43)c21 ; O=C1NCc2c(-c3cnc4cc(F)ccn34)ccc(Nc3ccc4c(n3)CNC[C@@H]3COCC[C@@H]43)c21 ; Cn1ccc2c(-c3ccc(Nc4ccc5c(n4)CNC[C@H]4COCCN54)c4c3CNC4=O)ccnc21]

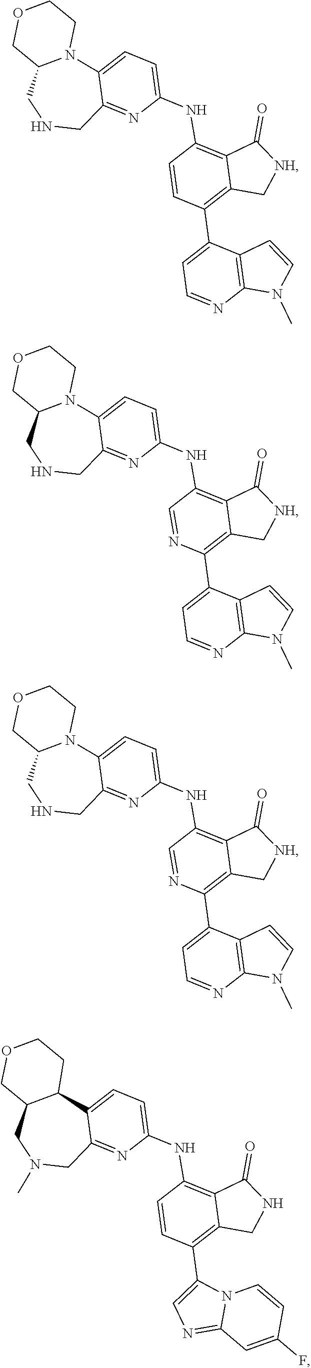 [SMILES: Cn1ccc2c(-c3ccc(Nc4ccc5c(n4)CNC[C@@H]4COCCN54)c4c3CNC4=O)ccnc21 ; Cn1ccc2c(-c3ncc(Nc4ccc5c(n4)CNC[C@H]4COCCN54)c4c3CNC4=O)ccnc21 ; Cn1ccc2c(-c3ncc(Nc4ccc5c(n4)CNC[C@@H]4COCCN54)c4c3CNC4=O)ccnc21 ; CN1Cc2nc(Nc3ccc(-c4cnc5cc(F)ccn45)c4c3C(=O)NC4)ccc2[C@@H]2CCOC[C@@H]2C1]

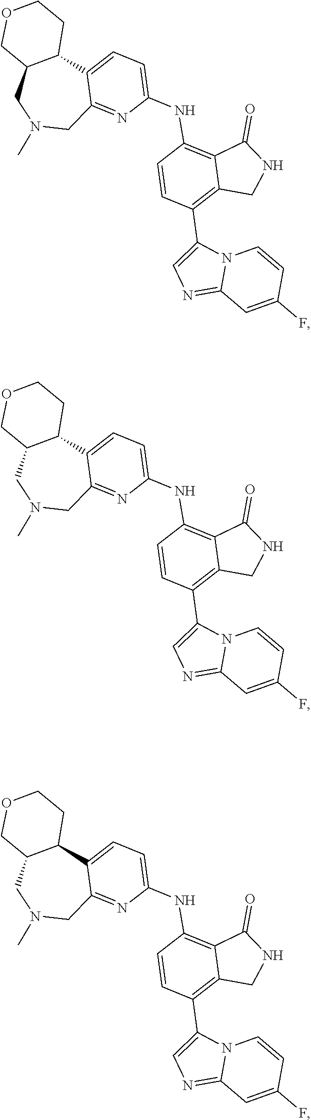 [SMILES: CN1Cc2nc(Nc3ccc(-c4cnc5cc(F)ccn45)c4c3C(=O)NC4)ccc2[C@H]2CCOC[C@@H]2C1 ; CN1Cc2nc(Nc3ccc(-c4cnc5cc(F)ccn45)c4c3C(=O)NC4)ccc2[C@H]2CCOC[C@H]2C1 ; CN1Cc2nc(Nc3ccc(-c4cnc5cc(F)ccn45)c4c3C(=O)NC4)ccc2[C@@H]2CCOC[C@H]2C1]

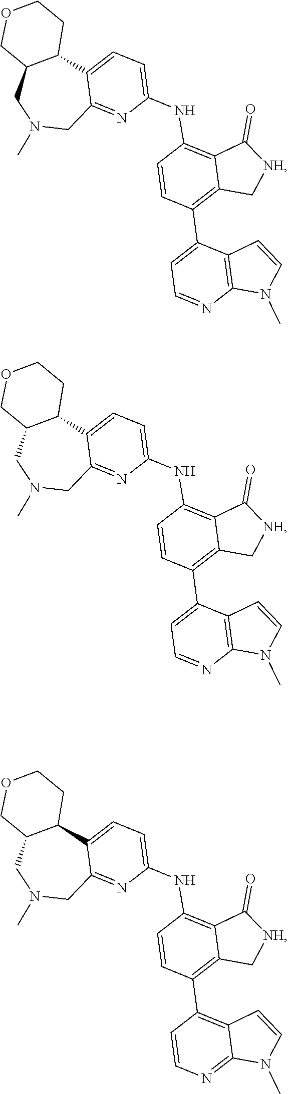 [SMILES: CN1Cc2nc(Nc3ccc(-c4ccnc5c4ccn5C)c4c3C(=O)NC4)ccc2[C@H]2CCOC[C@@H]2C1 ; CN1Cc2nc(Nc3ccc(-c4ccnc5c4ccn5C)c4c3C(=O)NC4)ccc2[C@H]2CCOC[C@H]2C1 ; CN1Cc2nc(Nc3ccc(-c4ccnc5c4ccn5C)c4c3C(=O)NC4)ccc2[C@@H]2CCOC[C@H]2C1]

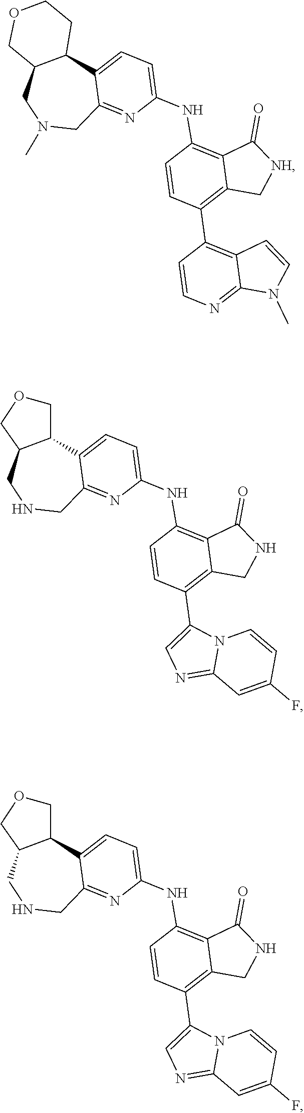 [SMILES: CN1Cc2nc(Nc3ccc(-c4ccnc5c4ccn5C)c4c3C(=O)NC4)ccc2[C@@H]2CCOC[C@@H]2C1 ; O=C1NCc2c(-c3cnc4cc(F)ccn34)ccc(Nc3ccc4c(n3)CNC[C@H]3COC[C@H]43)c21 ; O=C1NCc2c(-c3cnc4cc(F)ccn34)ccc(Nc3ccc4c(n3)CNC[C@@H]3COC[C@@H]43)c21]

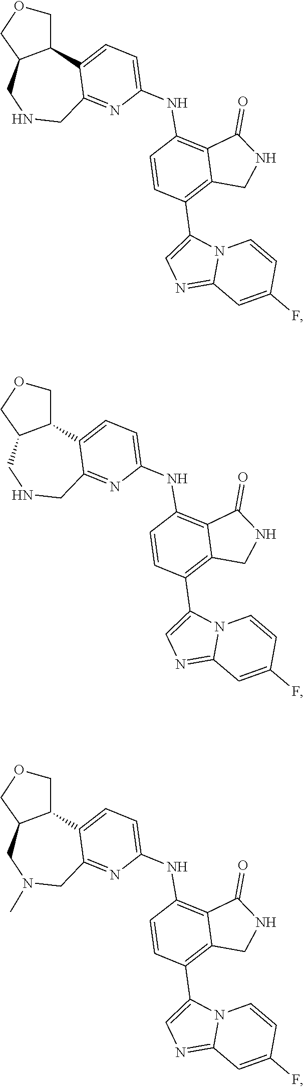 [SMILES: O=C1NCc2c(-c3cnc4cc(F)ccn34)ccc(Nc3ccc4c(n3)CNC[C@H]3COC[C@@H]43)c21 ; O=C1NCc2c(-c3cnc4cc(F)ccn34)ccc(Nc3ccc4c(n3)CNC[C@@H]3COC[C@H]43)c21 ; CN1Cc2nc(Nc3ccc(-c4cnc5cc(F)ccn45)c4c3C(=O)NC4)ccc2[C@H]2COC[C@@H]2C1]

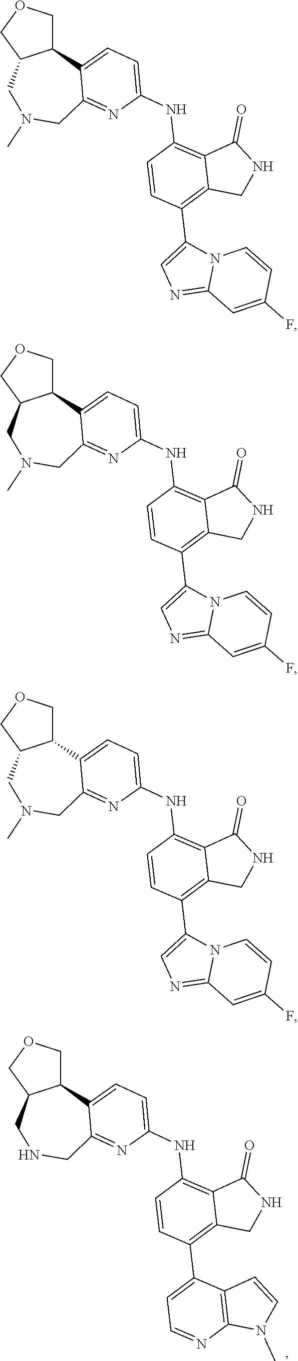 [SMILES: CN1Cc2nc(Nc3ccc(-c4cnc5cc(F)ccn45)c4c3C(=O)NC4)ccc2[C@@H]2COC[C@H]2C1 ; CN1Cc2nc(Nc3ccc(-c4cnc5cc(F)ccn45)c4c3C(=O)NC4)ccc2[C@@H]2COC[C@@H]2C1 ; CN1Cc2nc(Nc3ccc(-c4cnc5cc(F)ccn45)c4c3C(=O)NC4)ccc2[C@H]2COC[C@H]2C1 ; Cn1ccc2c(-c3ccc(Nc4ccc5c(n4)CNC[C@H]4COC[C@@H]54)c4c3CNC4=O)ccnc21]

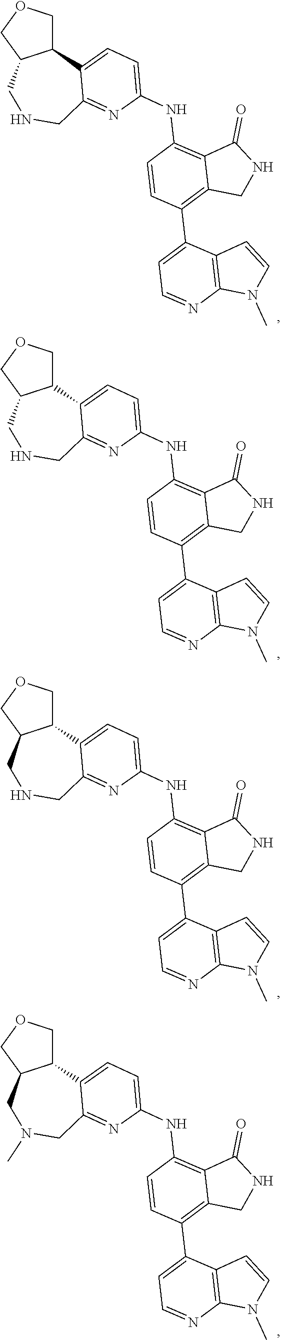

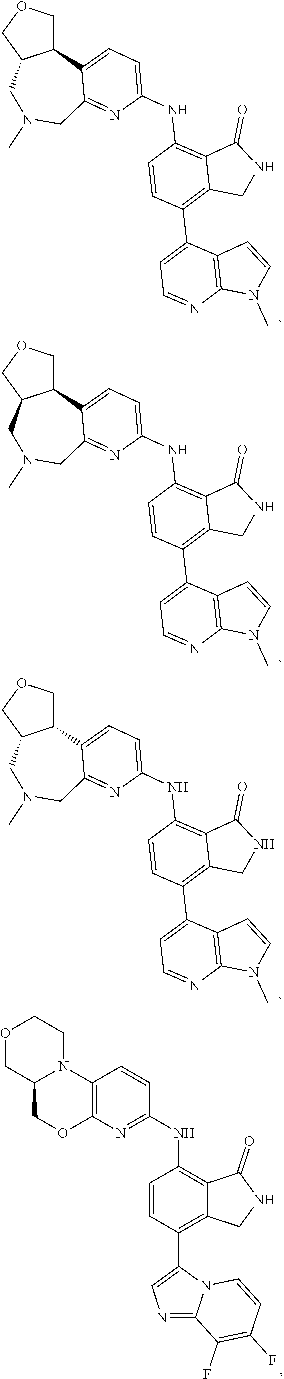 [SMILES: CN1Cc2nc(Nc3ccc(-c4ccnc5c4ccn5C)c4c3C(=O)NC4)ccc2[C@@H]2COC[C@H]2C1 ; CN1Cc2nc(Nc3ccc(-c4ccnc5c4ccn5C)c4c3C(=O)NC4)ccc2[C@@H]2COC[C@@H]2C1 ; CN1Cc2nc(Nc3ccc(-c4ccnc5c4ccn5C)c4c3C(=O)NC4)ccc2[C@H]2COC[C@H]2C1 ; O=C1NCc2c(-c3cnc4c(F)c(F)ccn34)ccc(Nc3ccc4c(n3)OC[C@H]3COCCN43)c21]

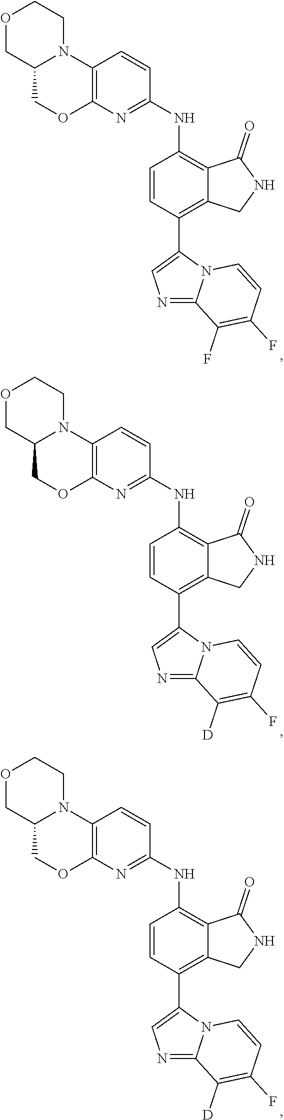 [SMILES: O=C1NCc2c(-c3cnc4c(F)c(F)ccn34)ccc(Nc3ccc4c(n3)OC[C@@H]3COCCN43)c21 ; [2H]c1c(F)ccn2c(-c3ccc(Nc4ccc5c(n4)OC[C@H]4COCCN54)c4c3CNC4=O)cnc12 ; [2H]c1c(F)ccn2c(-c3ccc(Nc4ccc5c(n4)OC[C@@H]4COCCN54)c4c3CNC4=O)cnc12]

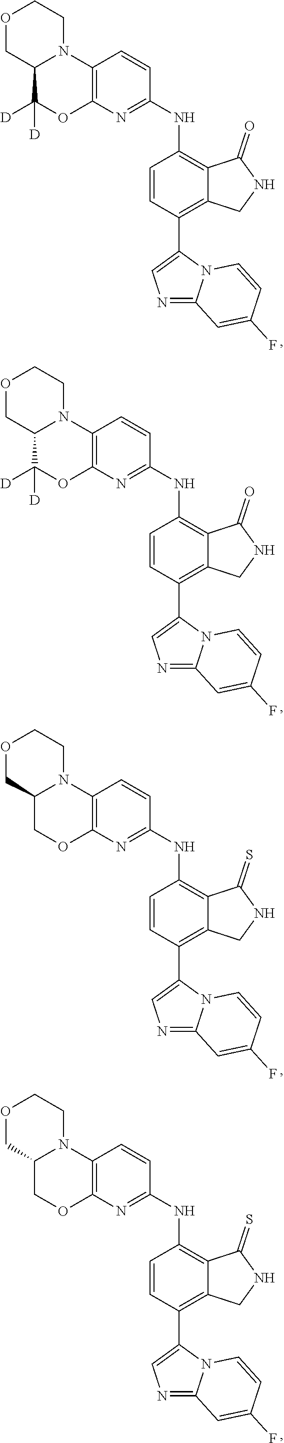 [SMILES: [2H]C1([2H])Oc2nc(Nc3ccc(-c4cnc5cc(F)ccn45)c4c3C(=O)NC4)ccc2N2CCOC[C@@H]21 ; [2H]C1([2H])Oc2nc(Nc3ccc(-c4cnc5cc(F)ccn45)c4c3C(=O)NC4)ccc2N2CCOC[C@H]21 ; Fc1ccn2c(-c3ccc(Nc4ccc5c(n4)OC[C@H]4COCCN54)c4c3CNC4=S)cnc2c1 ; Fc1ccn2c(-c3ccc(Nc4ccc5c(n4)OC[C@@H]4COCCN54)c4c3CNC4=S)cnc2c1]

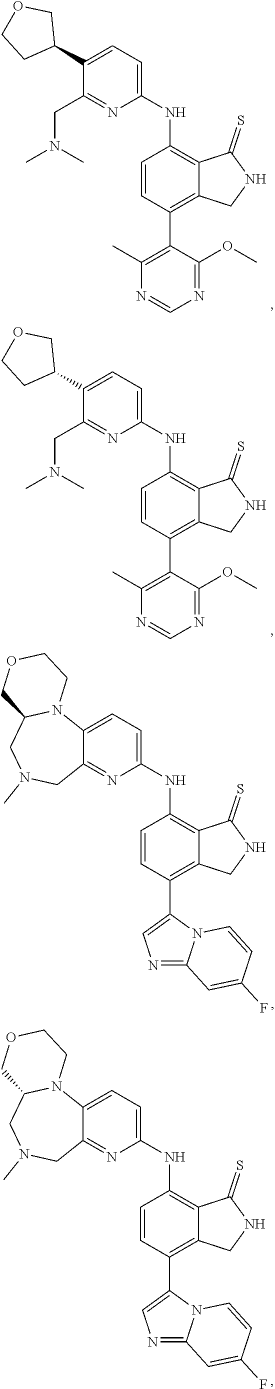 [SMILES: COc1ncnc(C)c1-c1ccc(Nc2ccc([C@H]3CCOC3)c(CN(C)C)n2)c2c1CNC2=S ; COc1ncnc(C)c1-c1ccc(Nc2ccc([C@@H]3CCOC3)c(CN(C)C)n2)c2c1CNC2=S ; CN1Cc2nc(Nc3ccc(-c4cnc5cc(F)ccn45)c4c3C(=S)NC4)ccc2N2CCOC[C@@H]2C1 ; CN1Cc2nc(Nc3ccc(-c4cnc5cc(F)ccn45)c4c3C(=S)NC4)ccc2N2CCOC[C@H]2C1]

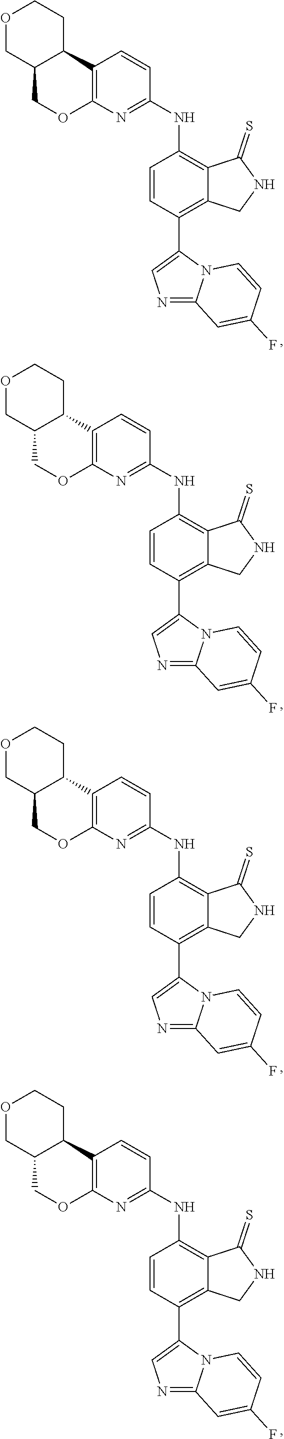 [SMILES: Fc1ccn2c(-c3ccc(Nc4ccc5c(n4)OC[C@H]4COCC[C@@H]54)c4c3CNC4=S)cnc2c1 ; Fc1ccn2c(-c3ccc(Nc4ccc5c(n4)OC[C@@H]4COCC[C@H]54)c4c3CNC4=S)cnc2c1 ; Fc1ccn2c(-c3ccc(Nc4ccc5c(n4)OC[C@H]4COCC[C@H]54)c4c3CNC4=S)cnc2c1 ; Fc1ccn2c(-c3ccc(Nc4ccc5c(n4)OC[C@@H]4COCC[C@@H]54)c4c3CNC4=S)cnc2c1]

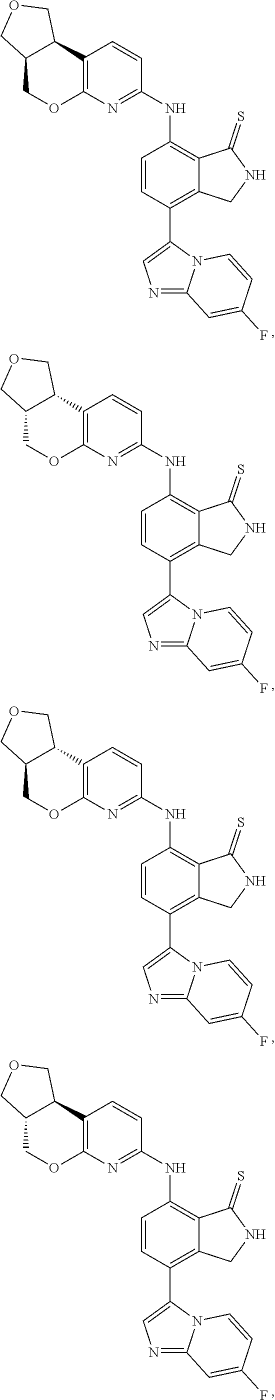 [SMILES: Fc1ccn2c(-c3ccc(Nc4ccc5c(n4)OC[C@H]4COC[C@@H]54)c4c3CNC4=S)cnc2c1 ; Fc1ccn2c(-c3ccc(Nc4ccc5c(n4)OC[C@@H]4COC[C@H]54)c4c3CNC4=S)cnc2c1 ; Fc1ccn2c(-c3ccc(Nc4ccc5c(n4)OC[C@H]4COC[C@H]54)c4c3CNC4=S)cnc2c1 ; Fc1ccn2c(-c3ccc(Nc4ccc5c(n4)OC[C@@H]4COC[C@@H]54)c4c3CNC4=S)cnc2c1]

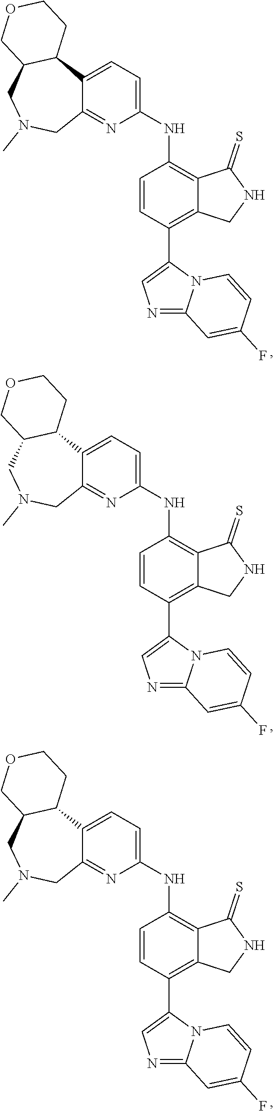 [SMILES: CN1Cc2nc(Nc3ccc(-c4cnc5cc(F)ccn45)c4c3C(=S)NC4)ccc2[C@@H]2CCOC[C@@H]2C1 ; CN1Cc2nc(Nc3ccc(-c4cnc5cc(F)ccn45)c4c3C(=S)NC4)ccc2[C@H]2CCOC[C@H]2C1 ; CN1Cc2nc(Nc3ccc(-c4cnc5cc(F)ccn45)c4c3C(=S)NC4)ccc2[C@H]2CCOC[C@@H]2C1]

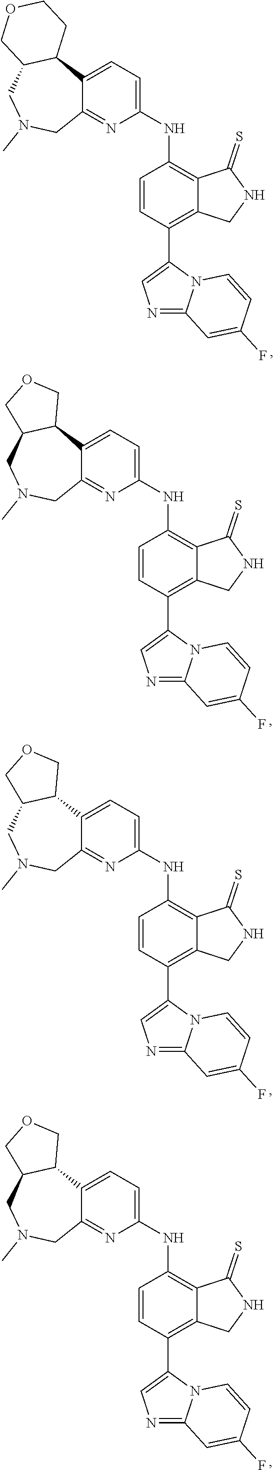 [SMILES: CN1Cc2nc(Nc3ccc(-c4cnc5cc(F)ccn45)c4c3C(=S)NC4)ccc2[C@@H]2CCOC[C@H]2C1 ; CN1Cc2nc(Nc3ccc(-c4cnc5cc(F)ccn45)c4c3C(=S)NC4)ccc2[C@@H]2COC[C@@H]2C1 ; CN1Cc2nc(Nc3ccc(-c4cnc5cc(F)ccn45)c4c3C(=S)NC4)ccc2[C@H]2COC[C@H]2C1 ; CN1Cc2nc(Nc3ccc(-c4cnc5cc(F)ccn45)c4c3C(=S)NC4)ccc2[C@H]2COC[C@@H]2C1]

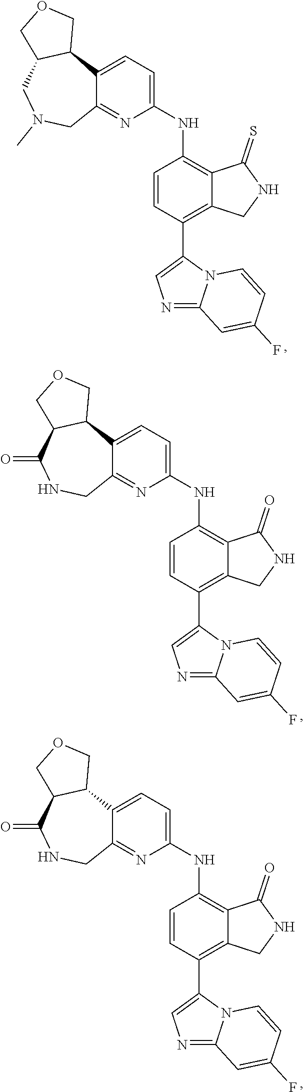 [SMILES: CN1Cc2nc(Nc3ccc(-c4cnc5cc(F)ccn45)c4c3C(=S)NC4)ccc2[C@@H]2COC[C@H]2C1 ; O=C1NCc2c(-c3cnc4cc(F)ccn34)ccc(Nc3ccc4c(n3)CNC(=O)[C@H]3COC[C@@H]43)c21 ; O=C1NCc2c(-c3cnc4cc(F)ccn34)ccc(Nc3ccc4c(n3)CNC(=O)[C@H]3COC[C@H]43)c21]

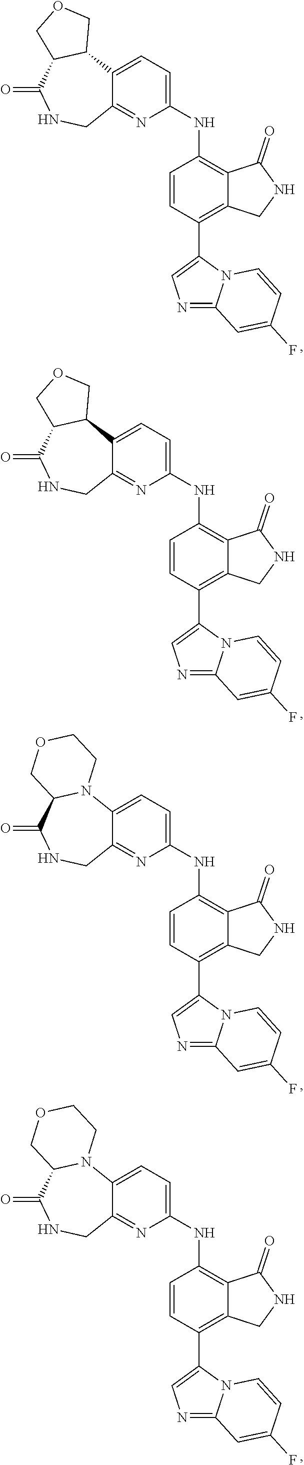 [SMILES: O=C1NCc2c(-c3cnc4cc(F)ccn34)ccc(Nc3ccc4c(n3)CNC(=O)[C@@H]3COC[C@H]43)c21 ; O=C1NCc2c(-c3cnc4cc(F)ccn34)ccc(Nc3ccc4c(n3)CNC(=O)[C@@H]3COC[C@@H]43)c21 ; O=C1NCc2c(-c3cnc4cc(F)ccn34)ccc(Nc3ccc4c(n3)CNC(=O)[C@H]3COCCN43)c21 ; O=C1NCc2c(-c3cnc4cc(F)ccn34)ccc(Nc3ccc4c(n3)CNC(=O)[C@@H]3COCCN43)c21]

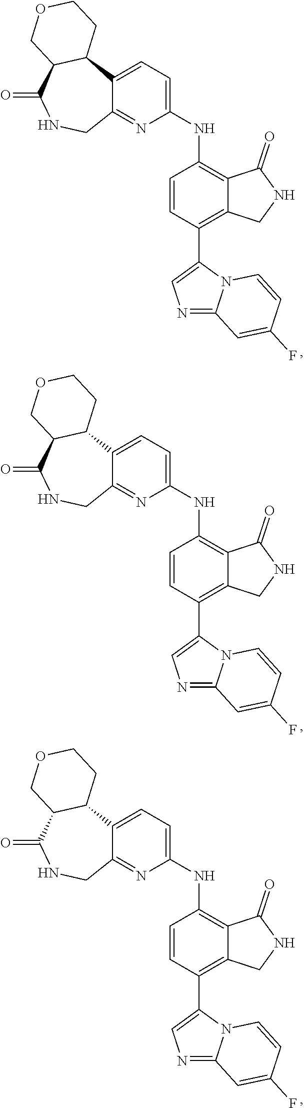 [SMILES: O=C1NCc2c(-c3cnc4cc(F)ccn34)ccc(Nc3ccc4c(n3)CNC(=O)[C@H]3COCC[C@@H]43)c21 ; O=C1NCc2c(-c3cnc4cc(F)ccn34)ccc(Nc3ccc4c(n3)CNC(=O)[C@H]3COCC[C@H]43)c21 ; O=C1NCc2c(-c3cnc4cc(F)ccn34)ccc(Nc3ccc4c(n3)CNC(=O)[C@@H]3COCC[C@H]43)c21]

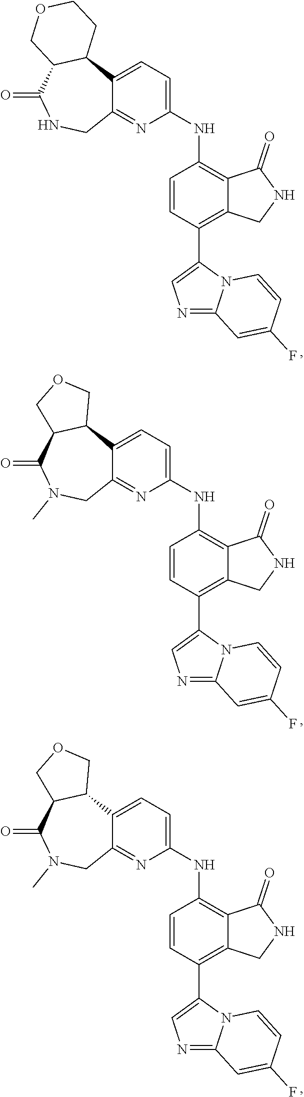 [SMILES: O=C1NCc2c(-c3cnc4cc(F)ccn34)ccc(Nc3ccc4c(n3)CNC(=O)[C@@H]3COCC[C@@H]43)c21 ; CN1Cc2nc(Nc3ccc(-c4cnc5cc(F)ccn45)c4c3C(=O)NC4)ccc2[C@@H]2COC[C@@H]2C1=O ; CN1Cc2nc(Nc3ccc(-c4cnc5cc(F)ccn45)c4c3C(=O)NC4)ccc2[C@H]2COC[C@@H]2C1=O]

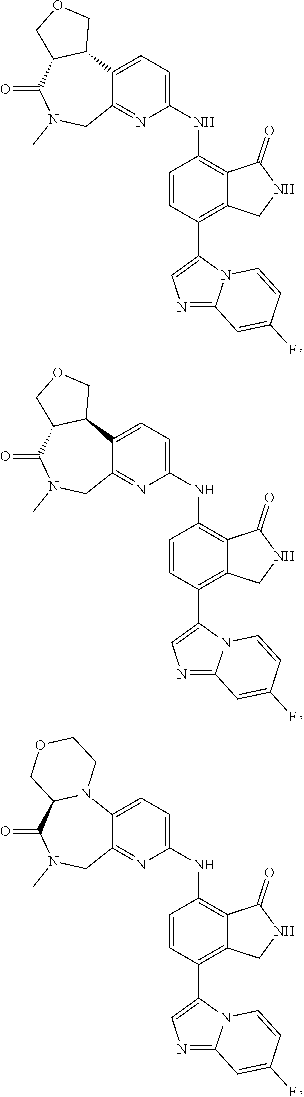 [SMILES: CN1Cc2nc(Nc3ccc(-c4cnc5cc(F)ccn45)c4c3C(=O)NC4)ccc2[C@H]2COC[C@H]2C1=O ; CN1Cc2nc(Nc3ccc(-c4cnc5cc(F)ccn45)c4c3C(=O)NC4)ccc2[C@@H]2COC[C@H]2C1=O ; CN1Cc2nc(Nc3ccc(-c4cnc5cc(F)ccn45)c4c3C(=O)NC4)ccc2N2CCOC[C@@H]2C1=O]

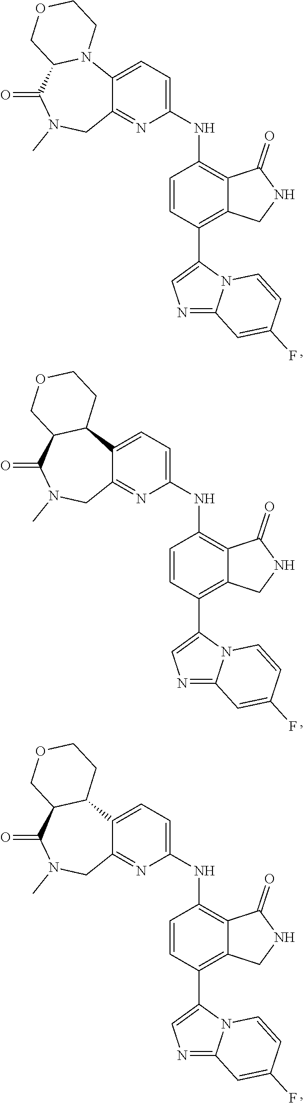 [SMILES: CN1Cc2nc(Nc3ccc(-c4cnc5cc(F)ccn45)c4c3C(=O)NC4)ccc2N2CCOC[C@H]2C1=O ; CN1Cc2nc(Nc3ccc(-c4cnc5cc(F)ccn45)c4c3C(=O)NC4)ccc2[C@@H]2CCOC[C@@H]2C1=O ; CN1Cc2nc(Nc3ccc(-c4cnc5cc(F)ccn45)c4c3C(=O)NC4)ccc2[C@H]2CCOC[C@@H]2C1=O]

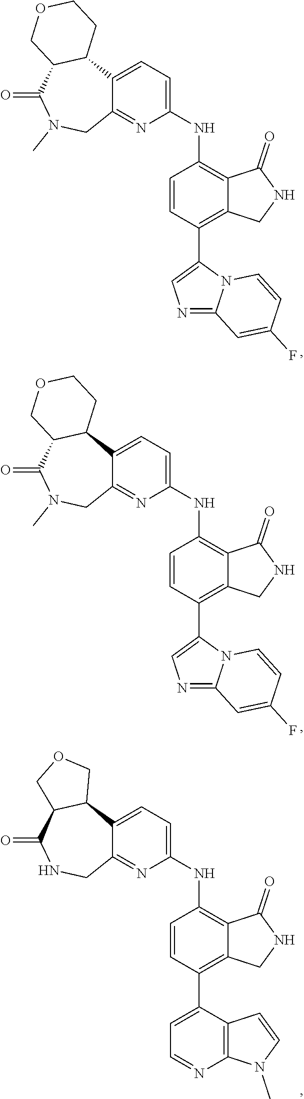 [SMILES: CN1Cc2nc(Nc3ccc(-c4cnc5cc(F)ccn45)c4c3C(=O)NC4)ccc2[C@H]2CCOC[C@H]2C1=O ; CN1Cc2nc(Nc3ccc(-c4cnc5cc(F)ccn45)c4c3C(=O)NC4)ccc2[C@@H]2CCOC[C@H]2C1=O ; Cn1ccc2c(-c3ccc(Nc4ccc5c(n4)CNC(=O)[C@H]4COC[C@@H]54)c4c3CNC4=O)ccnc21]

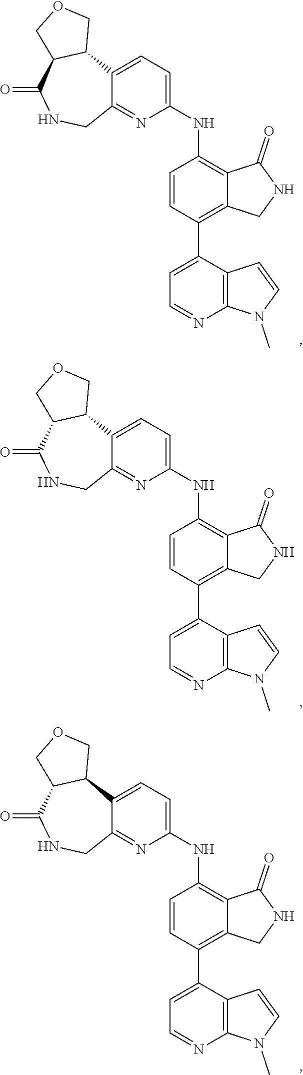 [SMILES: Cn1ccc2c(-c3ccc(Nc4ccc5c(n4)CNC(=O)[C@H]4COC[C@H]54)c4c3CNC4=O)ccnc21 ; Cn1ccc2c(-c3ccc(Nc4ccc5c(n4)CNC(=O)[C@@H]4COC[C@H]54)c4c3CNC4=O)ccnc21 ; Cn1ccc2c(-c3ccc(Nc4ccc5c(n4)CNC(=O)[C@@H]4COC[C@@H]54)c4c3CNC4=O)ccnc21]

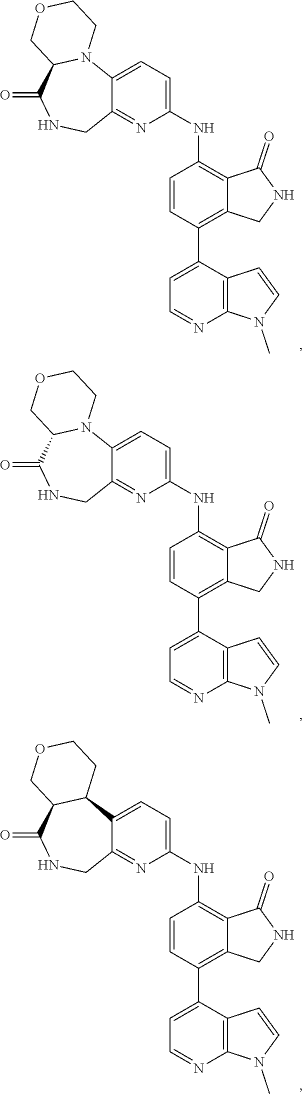 [SMILES: Cn1ccc2c(-c3ccc(Nc4ccc5c(n4)CNC(=O)[C@H]4COCCN54)c4c3CNC4=O)ccnc21 ; Cn1ccc2c(-c3ccc(Nc4ccc5c(n4)CNC(=O)[C@@H]4COCCN54)c4c3CNC4=O)ccnc21 ; Cn1ccc2c(-c3ccc(Nc4ccc5c(n4)CNC(=O)[C@H]4COCC[C@@H]54)c4c3CNC4=O)ccnc21]

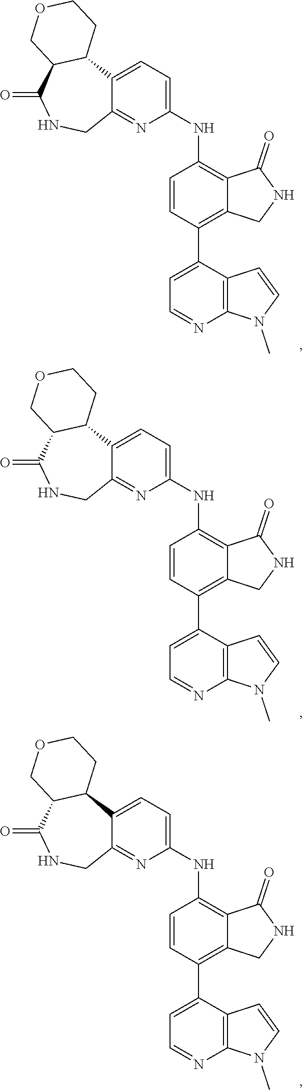 [SMILES: Cn1ccc2c(-c3ccc(Nc4ccc5c(n4)CNC(=O)[C@H]4COCC[C@H]54)c4c3CNC4=O)ccnc21 ; Cn1ccc2c(-c3ccc(Nc4ccc5c(n4)CNC(=O)[C@@H]4COCC[C@H]54)c4c3CNC4=O)ccnc21 ; Cn1ccc2c(-c3ccc(Nc4ccc5c(n4)CNC(=O)[C@@H]4COCC[C@@H]54)c4c3CNC4=O)ccnc21]

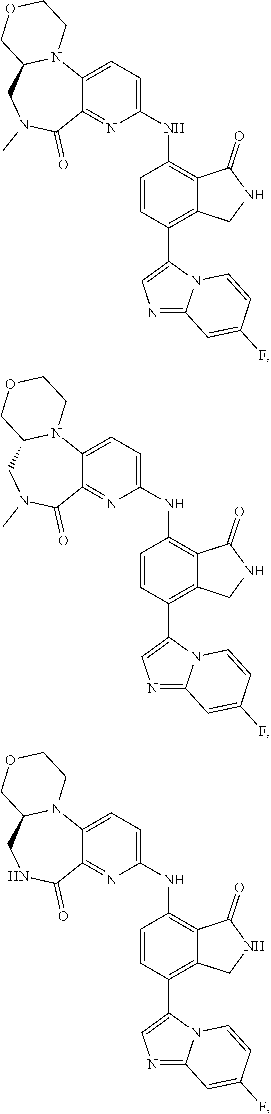 [SMILES: CN1C[C@H]2COCCN2c2ccc(Nc3ccc(-c4cnc5cc(F)ccn45)c4c3C(=O)NC4)nc2C1=O ; CN1C[C@@H]2COCCN2c2ccc(Nc3ccc(-c4cnc5cc(F)ccn45)c4c3C(=O)NC4)nc2C1=O ; O=C1NC[C@H]2COCCN2c2ccc(Nc3ccc(-c4cnc5cc(F)ccn45)c4c3C(=O)NC4)nc21]

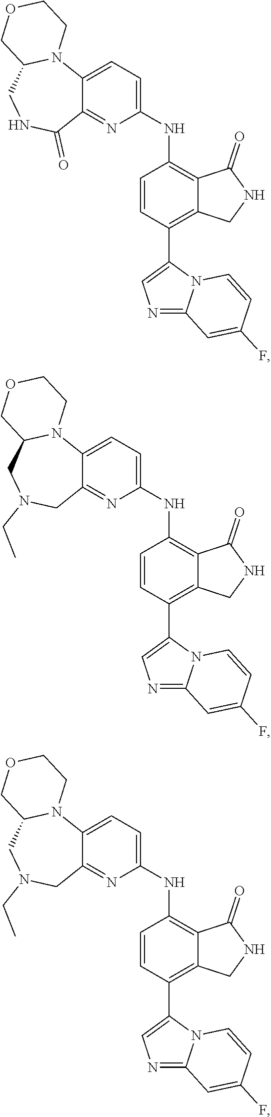 [SMILES: O=C1NC[C@@H]2COCCN2c2ccc(Nc3ccc(-c4cnc5cc(F)ccn45)c4c3C(=O)NC4)nc21 ; CCN1Cc2nc(Nc3ccc(-c4cnc5cc(F)ccn45)c4c3C(=O)NC4)ccc2N2CCOC[C@@H]2C1 ; CCN1Cc2nc(Nc3ccc(-c4cnc5cc(F)ccn45)c4c3C(=O)NC4)ccc2N2CCOC[C@H]2C1]

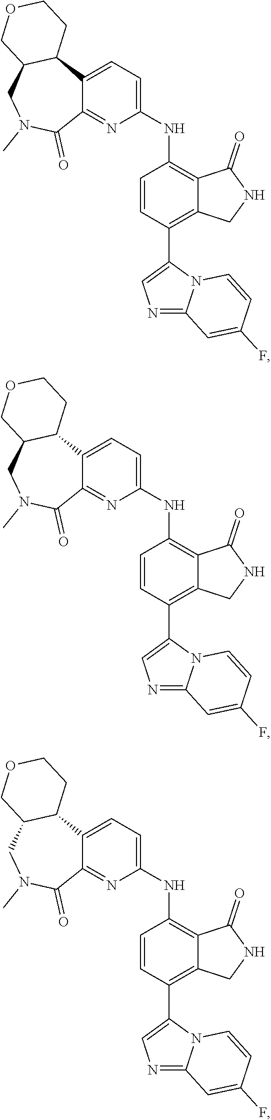 [SMILES: CN1C[C@H]2COCC[C@H]2c2ccc(Nc3ccc(-c4cnc5cc(F)ccn45)c4c3C(=O)NC4)nc2C1=O ; CN1C[C@H]2COCC[C@@H]2c2ccc(Nc3ccc(-c4cnc5cc(F)ccn45)c4c3C(=O)NC4)nc2C1=O ; CN1C[C@@H]2COCC[C@@H]2c2ccc(Nc3ccc(-c4cnc5cc(F)ccn45)c4c3C(=O)NC4)nc2C1=O]

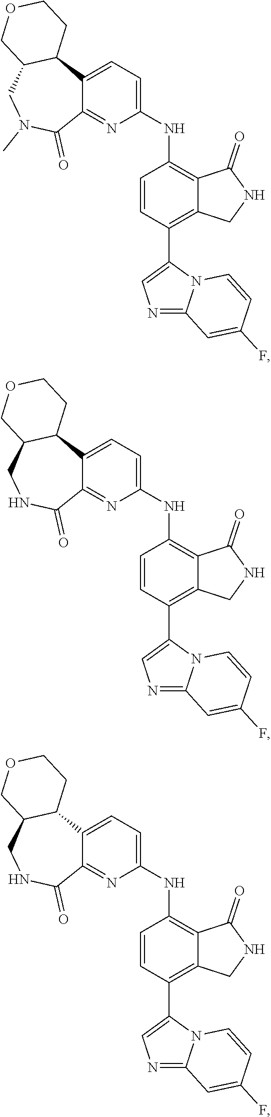 [SMILES: CN1C[C@@H]2COCC[C@H]2c2ccc(Nc3ccc(-c4cnc5cc(F)ccn45)c4c3C(=O)NC4)nc2C1=O ; O=C1NC[C@H]2COCC[C@H]2c2ccc(Nc3ccc(-c4cnc5cc(F)ccn45)c4c3C(=O)NC4)nc21 ; O=C1NC[C@H]2COCC[C@@H]2c2ccc(Nc3ccc(-c4cnc5cc(F)ccn45)c4c3C(=O)NC4)nc21]

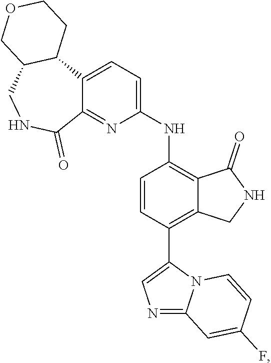 [SMILES: O=C1NC[C@@H]2COCC[C@@H]2c2ccc(Nc3ccc(-c4cnc5cc(F)ccn45)c4c3C(=O)NC4)nc21]

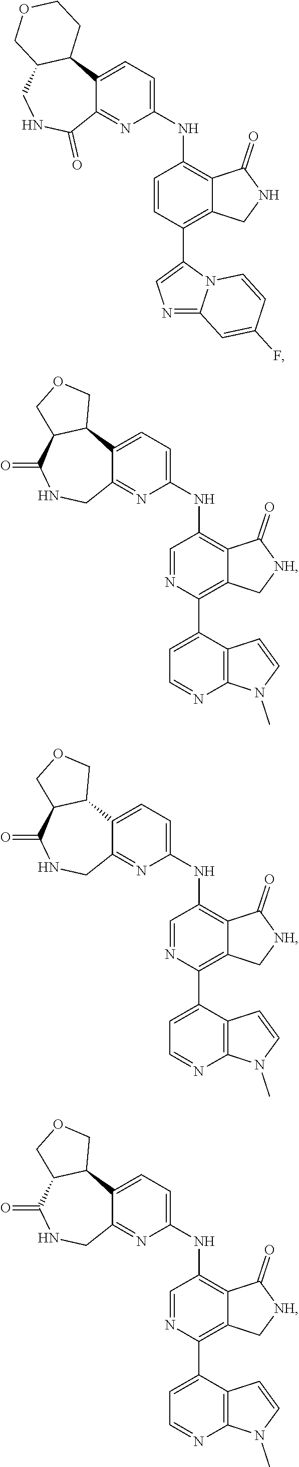 [SMILES: O=C1NC[C@@H]2COCC[C@H]2c2ccc(Nc3ccc(-c4cnc5cc(F)ccn45)c4c3C(=O)NC4)nc21 ; Cn1ccc2c(-c3ncc(Nc4ccc5c(n4)CNC(=O)[C@H]4COC[C@@H]54)c4c3CNC4=O)ccnc21 ; Cn1ccc2c(-c3ncc(Nc4ccc5c(n4)CNC(=O)[C@H]4COC[C@H]54)c4c3CNC4=O)ccnc21 ; Cn1ccc2c(-c3ncc(Nc4ccc5c(n4)CNC(=O)[C@@H]4COC[C@@H]54)c4c3CNC4=O)ccnc21]

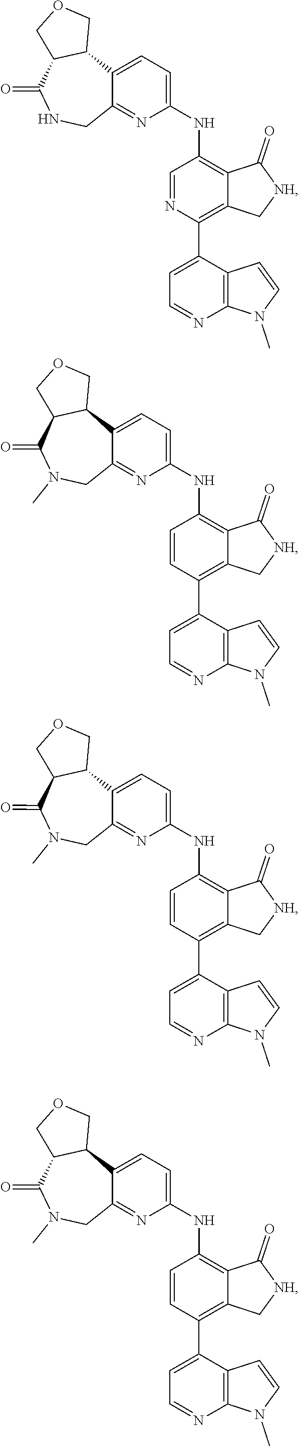 [SMILES: Cn1ccc2c(-c3ncc(Nc4ccc5c(n4)CNC(=O)[C@@H]4COC[C@H]54)c4c3CNC4=O)ccnc21 ; CN1Cc2nc(Nc3ccc(-c4ccnc5c4ccn5C)c4c3C(=O)NC4)ccc2[C@@H]2COC[C@@H]2C1=O ; CN1Cc2nc(Nc3ccc(-c4ccnc5c4ccn5C)c4c3C(=O)NC4)ccc2[C@H]2COC[C@@H]2C1=O ; CN1Cc2nc(Nc3ccc(-c4ccnc5c4ccn5C)c4c3C(=O)NC4)ccc2[C@@H]2COC[C@H]2C1=O]

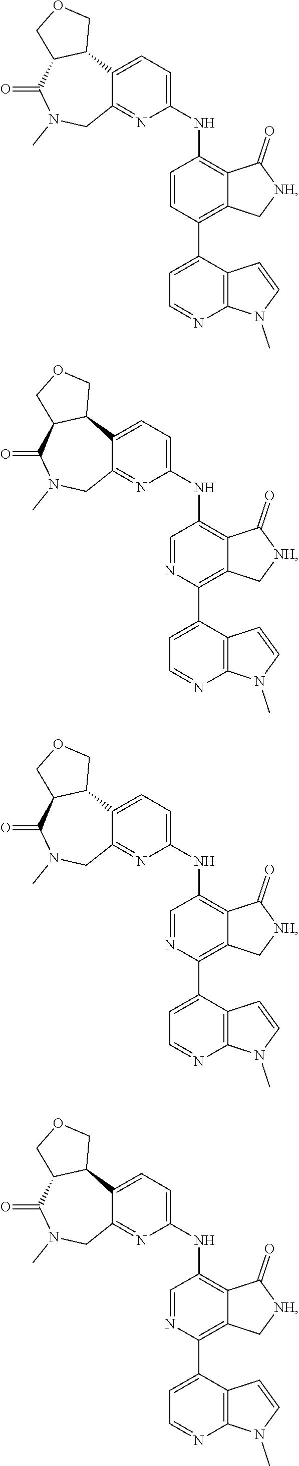 [SMILES: CN1Cc2nc(Nc3ccc(-c4ccnc5c4ccn5C)c4c3C(=O)NC4)ccc2[C@H]2COC[C@H]2C1=O ; CN1Cc2nc(Nc3cnc(-c4ccnc5c4ccn5C)c4c3C(=O)NC4)ccc2[C@@H]2COC[C@@H]2C1=O ; CN1Cc2nc(Nc3cnc(-c4ccnc5c4ccn5C)c4c3C(=O)NC4)ccc2[C@H]2COC[C@@H]2C1=O ; CN1Cc2nc(Nc3cnc(-c4ccnc5c4ccn5C)c4c3C(=O)NC4)ccc2[C@@H]2COC[C@H]2C1=O]

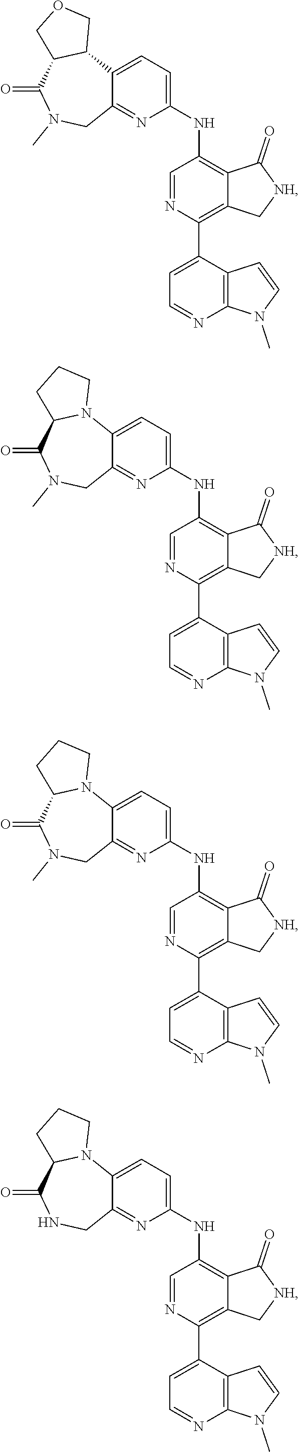 [SMILES: CN1Cc2nc(Nc3cnc(-c4ccnc5c4ccn5C)c4c3C(=O)NC4)ccc2[C@H]2COC[C@H]2C1=O ; CN1Cc2nc(Nc3cnc(-c4ccnc5c4ccn5C)c4c3C(=O)NC4)ccc2N2CCC[C@@H]2C1=O ; CN1Cc2nc(Nc3cnc(-c4ccnc5c4ccn5C)c4c3C(=O)NC4)ccc2N2CCC[C@H]2C1=O ; Cn1ccc2c(-c3ncc(Nc4ccc5c(n4)CNC(=O)[C@H]4CCCN54)c4c3CNC4=O)ccnc21]

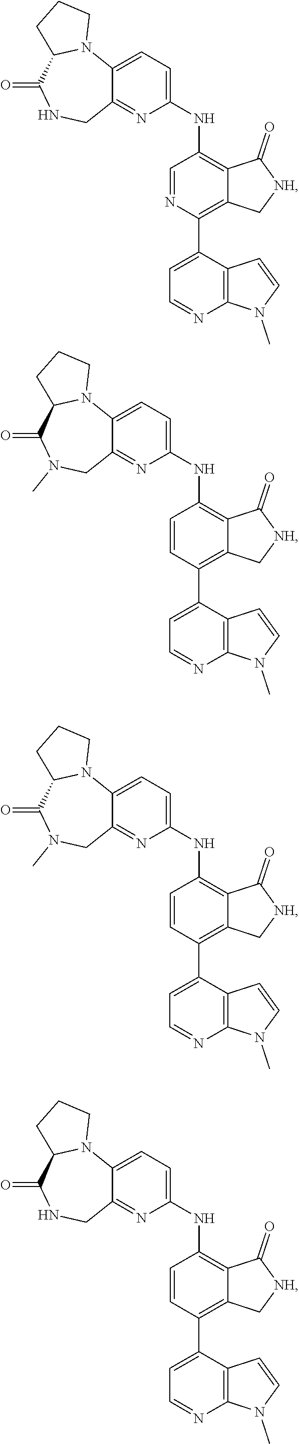 [SMILES: Cn1ccc2c(-c3ncc(Nc4ccc5c(n4)CNC(=O)[C@@H]4CCCN54)c4c3CNC4=O)ccnc21 ; CN1Cc2nc(Nc3ccc(-c4ccnc5c4ccn5C)c4c3C(=O)NC4)ccc2N2CCC[C@@H]2C1=O ; CN1Cc2nc(Nc3ccc(-c4ccnc5c4ccn5C)c4c3C(=O)NC4)ccc2N2CCC[C@H]2C1=O ; Cn1ccc2c(-c3ccc(Nc4ccc5c(n4)CNC(=O)[C@H]4CCCN54)c4c3CNC4=O)ccnc21]

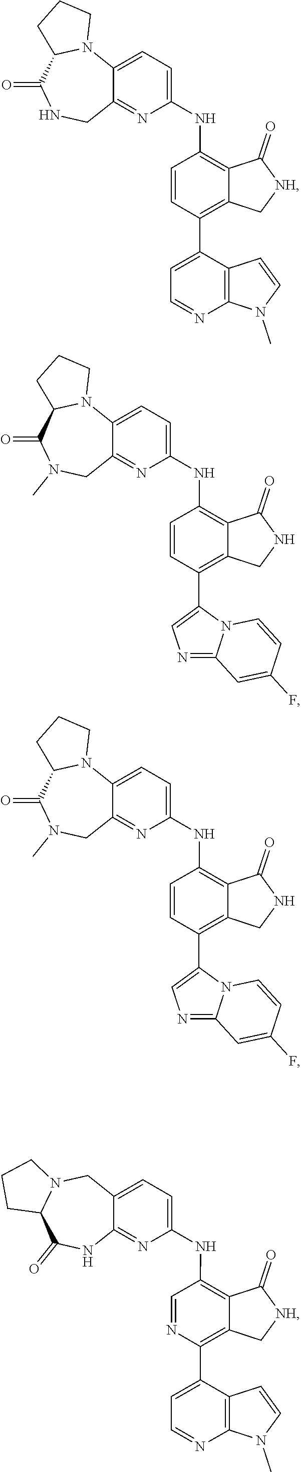 [SMILES: Cn1ccc2c(-c3ccc(Nc4ccc5c(n4)CNC(=O)[C@@H]4CCCN54)c4c3CNC4=O)ccnc21 ; CN1Cc2nc(Nc3ccc(-c4cnc5cc(F)ccn45)c4c3C(=O)NC4)ccc2N2CCC[C@@H]2C1=O ; CN1Cc2nc(Nc3ccc(-c4cnc5cc(F)ccn45)c4c3C(=O)NC4)ccc2N2CCC[C@H]2C1=O ; Cn1ccc2c(-c3ncc(Nc4ccc5c(n4)NC(=O)[C@H]4CCCN4C5)c4c3CNC4=O)ccnc21]

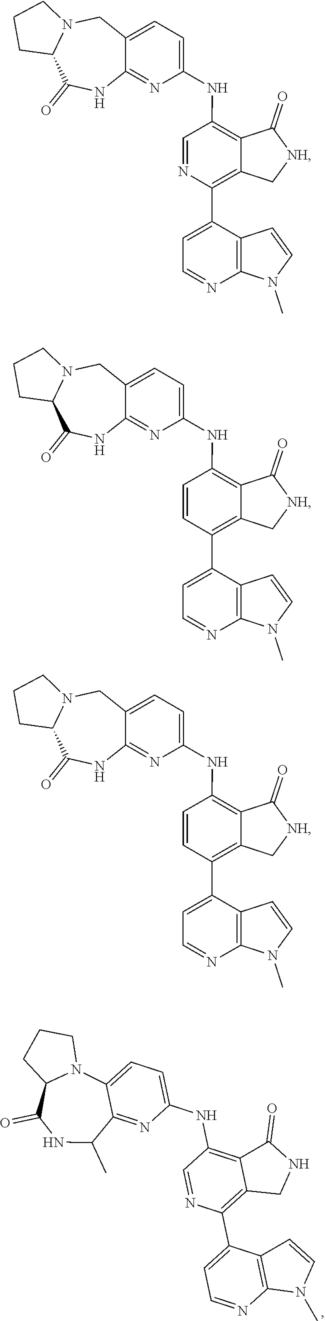

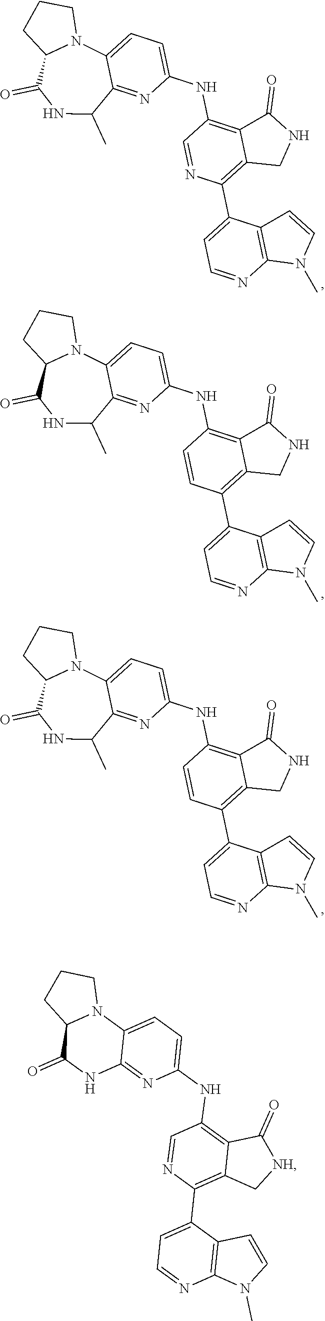

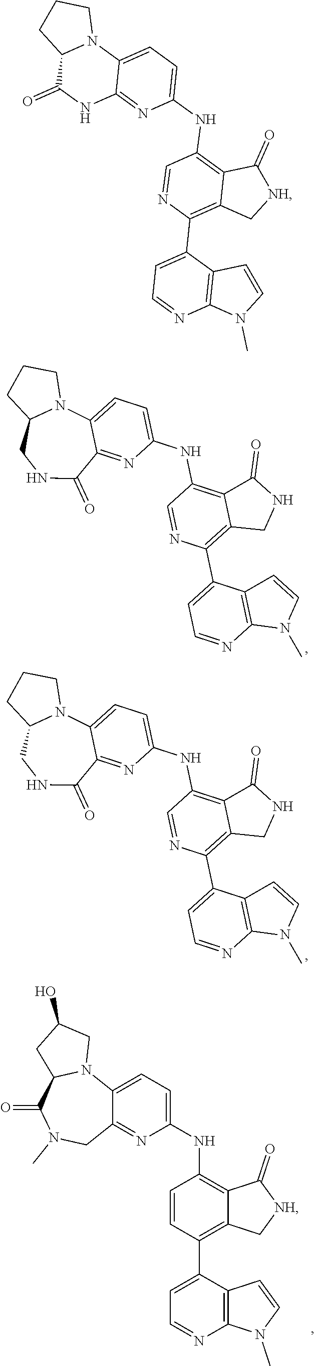 [SMILES: Cn1ccc2c(-c3ncc(Nc4ccc5c(n4)NC(=O)[C@@H]4CCCN54)c4c3CNC4=O)ccnc21 ; Cn1ccc2c(-c3ncc(Nc4ccc5c(n4)C(=O)NC[C@H]4CCCN54)c4c3CNC4=O)ccnc21 ; Cn1ccc2c(-c3ncc(Nc4ccc5c(n4)C(=O)NC[C@@H]4CCCN54)c4c3CNC4=O)ccnc21 ; CN1Cc2nc(Nc3ccc(-c4ccnc5c4ccn5C)c4c3C(=O)NC4)ccc2N2C[C@H](O)C[C@@H]2C1=O]

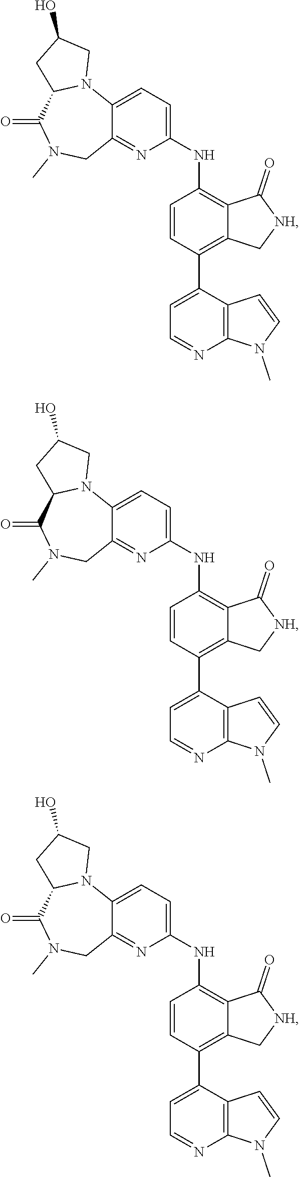 [SMILES: CN1Cc2nc(Nc3ccc(-c4ccnc5c4ccn5C)c4c3C(=O)NC4)ccc2N2C[C@H](O)C[C@H]2C1=O ; CN1Cc2nc(Nc3ccc(-c4ccnc5c4ccn5C)c4c3C(=O)NC4)ccc2N2C[C@@H](O)C[C@@H]2C1=O ; CN1Cc2nc(Nc3ccc(-c4ccnc5c4ccn5C)c4c3C(=O)NC4)ccc2N2C[C@@H](O)C[C@H]2C1=O]

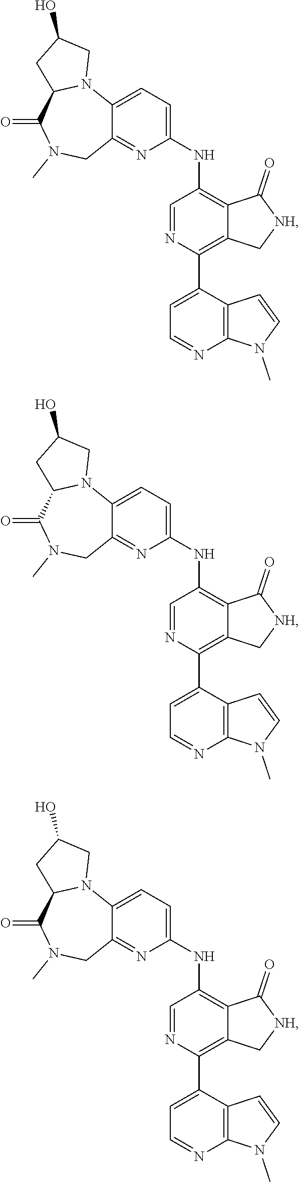 [SMILES: CN1Cc2nc(Nc3cnc(-c4ccnc5c4ccn5C)c4c3C(=O)NC4)ccc2N2C[C@H](O)C[C@@H]2C1=O ; CN1Cc2nc(Nc3cnc(-c4ccnc5c4ccn5C)c4c3C(=O)NC4)ccc2N2C[C@H](O)C[C@H]2C1=O ; CN1Cc2nc(Nc3cnc(-c4ccnc5c4ccn5C)c4c3C(=O)NC4)ccc2N2C[C@@H](O)C[C@@H]2C1=O]

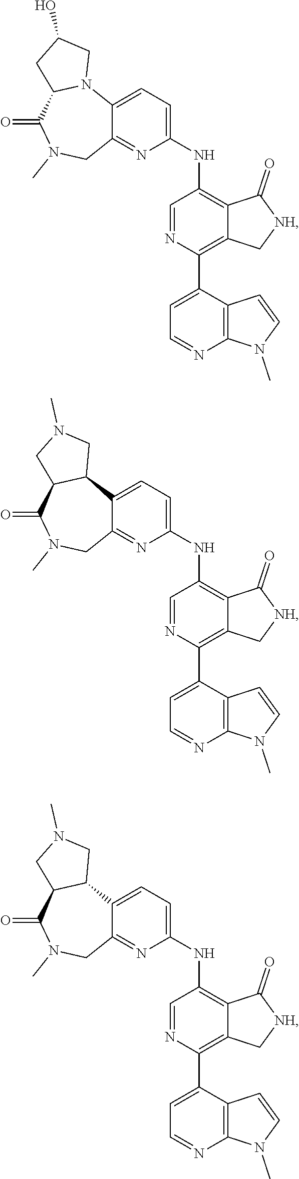 [SMILES: CN1Cc2nc(Nc3cnc(-c4ccnc5c4ccn5C)c4c3C(=O)NC4)ccc2N2C[C@@H](O)C[C@H]2C1=O ; CN1C[C@@H]2C(=O)N(C)Cc3nc(Nc4cnc(-c5ccnc6c5ccn6C)c5c4C(=O)NC5)ccc3[C@@H]2C1 ; CN1C[C@@H]2C(=O)N(C)Cc3nc(Nc4cnc(-c5ccnc6c5ccn6C)c5c4C(=O)NC5)ccc3[C@H]2C1]

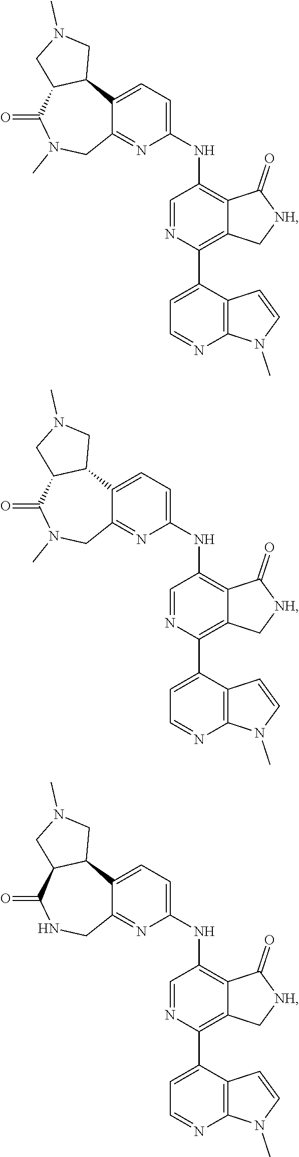 [SMILES: CN1C[C@H]2C(=O)N(C)Cc3nc(Nc4cnc(-c5ccnc6c5ccn6C)c5c4C(=O)NC5)ccc3[C@@H]2C1 ; CN1C[C@@H]2c3ccc(Nc4cnc(-c5ccnc6c5ccn6C)c5c4C(=O)NC5)nc3CN(C)C(=O)[C@@H]2C1 ; CN1C[C@@H]2C(=O)NCc3nc(Nc4cnc(-c5ccnc6c5ccn6C)c5c4C(=O)NC5)ccc3[C@@H]2C1]

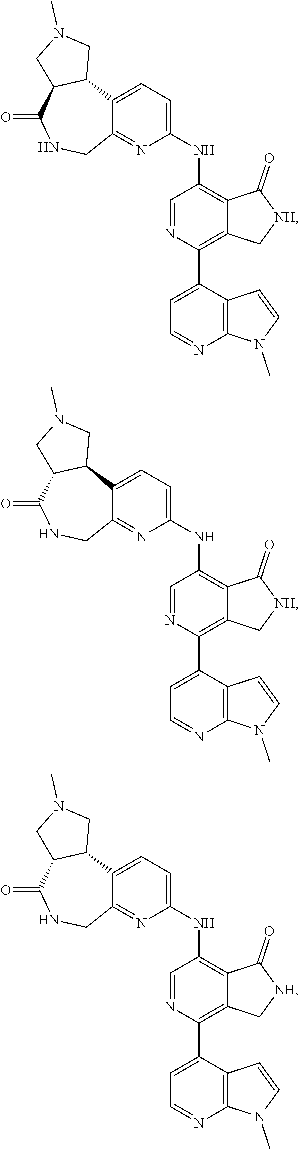 [SMILES: CN1C[C@@H]2C(=O)NCc3nc(Nc4cnc(-c5ccnc6c5ccn6C)c5c4C(=O)NC5)ccc3[C@H]2C1 ; CN1C[C@H]2C(=O)NCc3nc(Nc4cnc(-c5ccnc6c5ccn6C)c5c4C(=O)NC5)ccc3[C@@H]2C1 ; CN1C[C@@H]2c3ccc(Nc4cnc(-c5ccnc6c5ccn6C)c5c4C(=O)NC5)nc3CNC(=O)[C@@H]2C1]

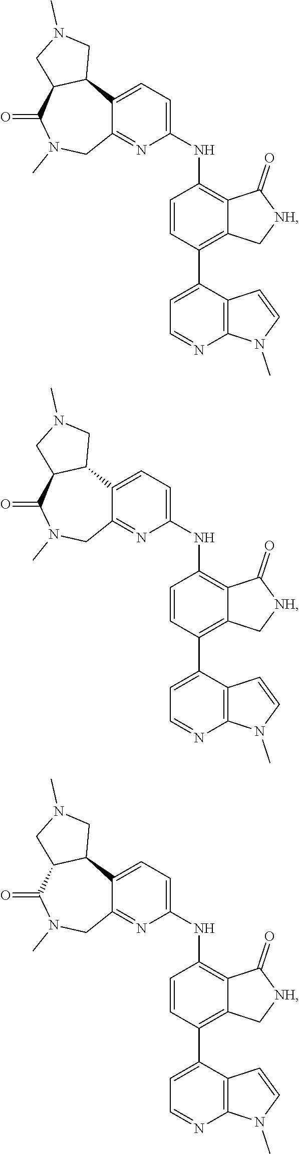 [SMILES: CN1C[C@@H]2C(=O)N(C)Cc3nc(Nc4ccc(-c5ccnc6c5ccn6C)c5c4C(=O)NC5)ccc3[C@@H]2C1 ; CN1C[C@@H]2C(=O)N(C)Cc3nc(Nc4ccc(-c5ccnc6c5ccn6C)c5c4C(=O)NC5)ccc3[C@H]2C1 ; CN1C[C@H]2C(=O)N(C)Cc3nc(Nc4ccc(-c5ccnc6c5ccn6C)c5c4C(=O)NC5)ccc3[C@@H]2C1]

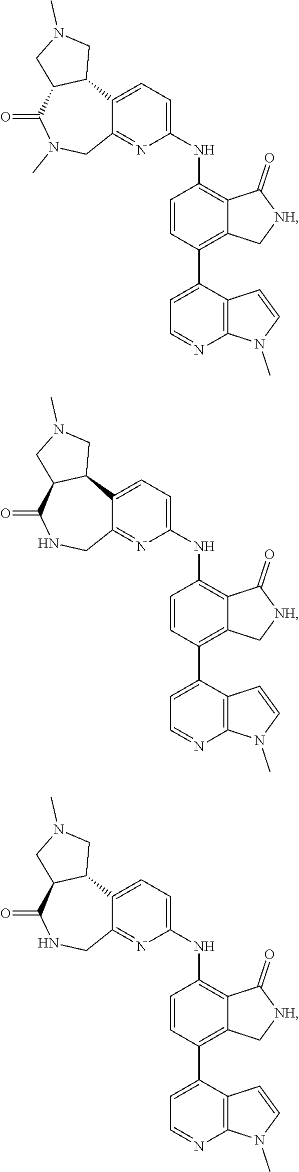 [SMILES: CN1C[C@@H]2c3ccc(Nc4ccc(-c5ccnc6c5ccn6C)c5c4C(=O)NC5)nc3CN(C)C(=O)[C@@H]2C1 ; CN1C[C@@H]2C(=O)NCc3nc(Nc4ccc(-c5ccnc6c5ccn6C)c5c4C(=O)NC5)ccc3[C@@H]2C1 ; CN1C[C@@H]2C(=O)NCc3nc(Nc4ccc(-c5ccnc6c5ccn6C)c5c4C(=O)NC5)ccc3[C@H]2C1]

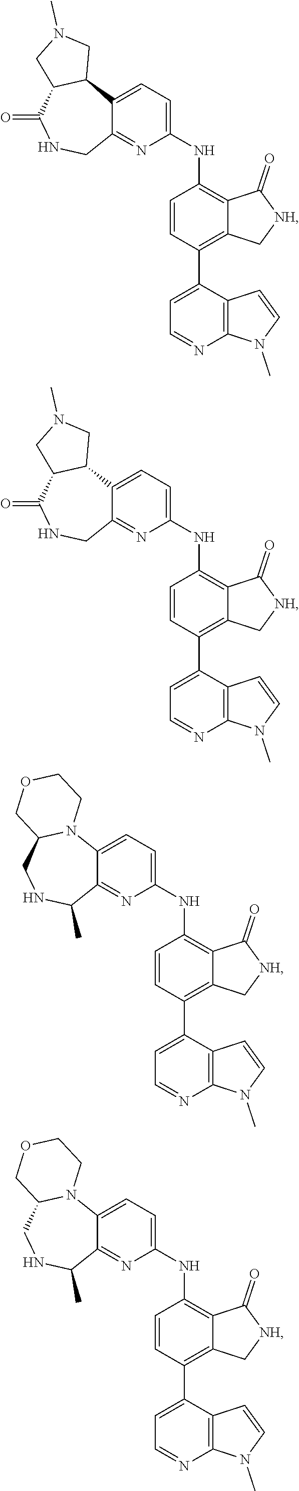 [SMILES: CN1C[C@H]2C(=O)NCc3nc(Nc4ccc(-c5ccnc6c5ccn6C)c5c4C(=O)NC5)ccc3[C@@H]2C1 ; CN1C[C@@H]2c3ccc(Nc4ccc(-c5ccnc6c5ccn6C)c5c4C(=O)NC5)nc3CNC(=O)[C@@H]2C1 ; C[C@H]1NC[C@H]2COCCN2c2ccc(Nc3ccc(-c4ccnc5c4ccn5C)c4c3C(=O)NC4)nc21 ; C[C@H]1NC[C@@H]2COCCN2c2ccc(Nc3ccc(-c4ccnc5c4ccn5C)c4c3C(=O)NC4)nc21]

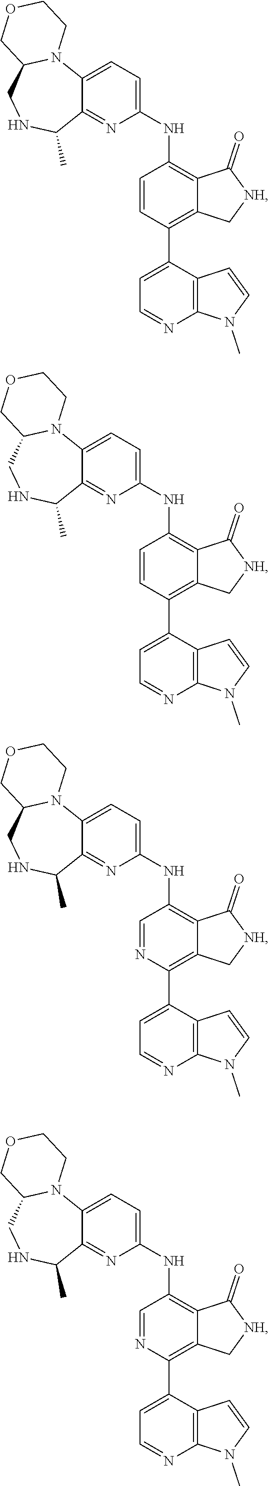 [SMILES: C[C@@H]1NC[C@H]2COCCN2c2ccc(Nc3ccc(-c4ccnc5c4ccn5C)c4c3C(=O)NC4)nc21 ; C[C@@H]1NC[C@@H]2COCCN2c2ccc(Nc3ccc(-c4ccnc5c4ccn5C)c4c3C(=O)NC4)nc21 ; C[C@H]1NC[C@H]2COCCN2c2ccc(Nc3cnc(-c4ccnc5c4ccn5C)c4c3C(=O)NC4)nc21 ; C[C@H]1NC[C@@H]2COCCN2c2ccc(Nc3cnc(-c4ccnc5c4ccn5C)c4c3C(=O)NC4)nc21]

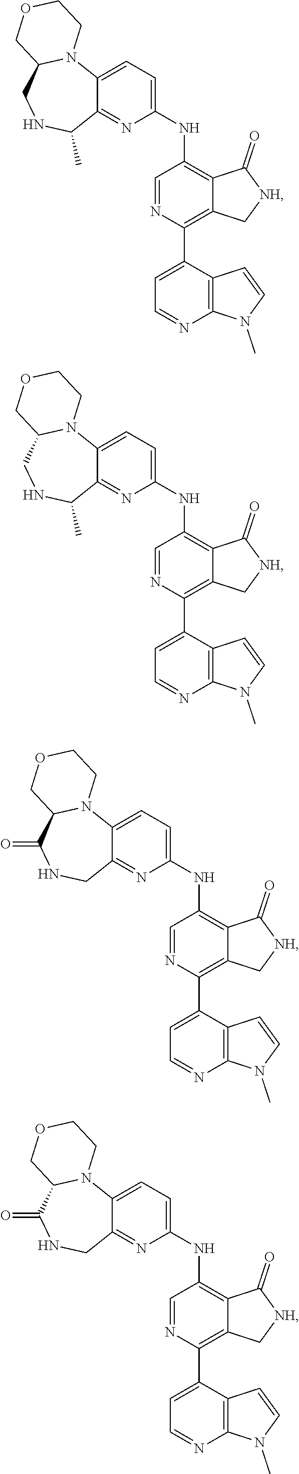 [SMILES: C[C@@H]1NC[C@H]2COCCN2c2ccc(Nc3cnc(-c4ccnc5c4ccn5C)c4c3C(=O)NC4)nc21 ; C[C@@H]1NC[C@@H]2COCCN2c2ccc(Nc3cnc(-c4ccnc5c4ccn5C)c4c3C(=O)NC4)nc21 ; Cn1ccc2c(-c3ncc(Nc4ccc5c(n4)CNC(=O)[C@H]4COCCN54)c4c3CNC4=O)ccnc21 ; Cn1ccc2c(-c3ncc(Nc4ccc5c(n4)CNC(=O)[C@@H]4COCCN54)c4c3CNC4=O)ccnc21]

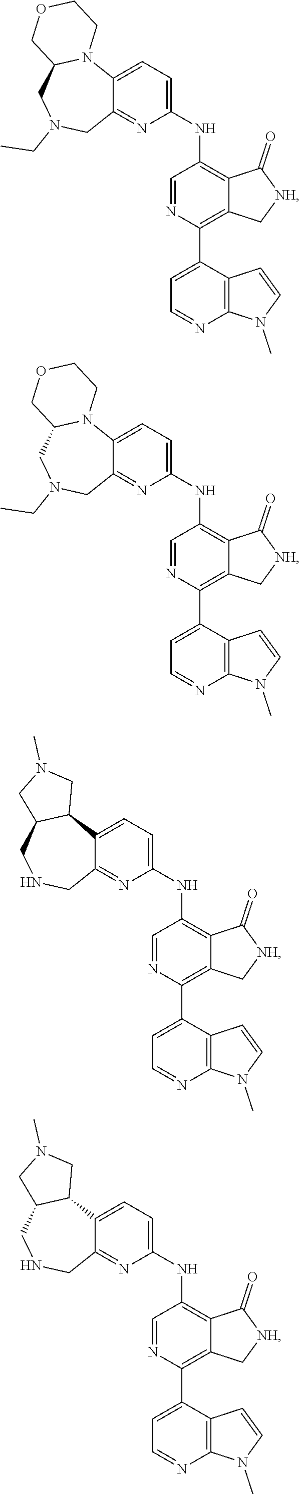 [SMILES: CCN1Cc2nc(Nc3cnc(-c4ccnc5c4ccn5C)c4c3C(=O)NC4)ccc2N2CCOC[C@@H]2C1 ; CCN1Cc2nc(Nc3cnc(-c4ccnc5c4ccn5C)c4c3C(=O)NC4)ccc2N2CCOC[C@H]2C1 ; CN1C[C@@H]2CNCc3nc(Nc4cnc(-c5ccnc6c5ccn6C)c5c4C(=O)NC5)ccc3[C@@H]2C1 ; CN1C[C@H]2CNCc3nc(Nc4cnc(-c5ccnc6c5ccn6C)c5c4C(=O)NC5)ccc3[C@H]2C1]

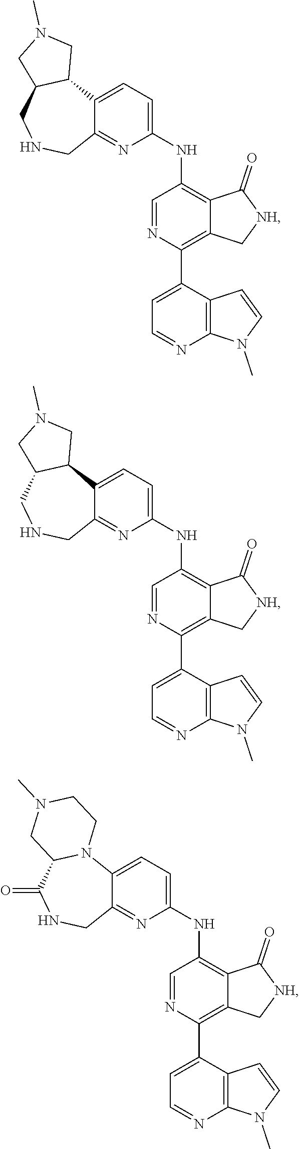 [SMILES: CN1C[C@@H]2CNCc3nc(Nc4cnc(-c5ccnc6c5ccn6C)c5c4C(=O)NC5)ccc3[C@H]2C1 ; CN1C[C@H]2CNCc3nc(Nc4cnc(-c5ccnc6c5ccn6C)c5c4C(=O)NC5)ccc3[C@@H]2C1 ; CN1CCN2c3ccc(Nc4cnc(-c5ccnc6c5ccn6C)c5c4C(=O)NC5)nc3CNC(=O)[C@@H]2C1]

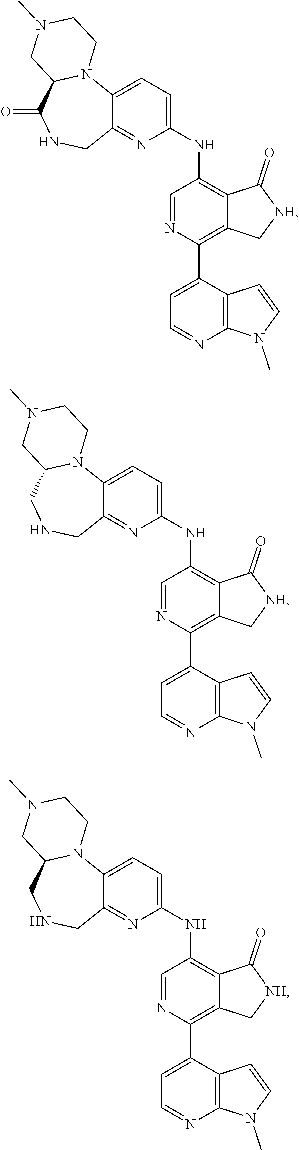 [SMILES: CN1CCN2c3ccc(Nc4cnc(-c5ccnc6c5ccn6C)c5c4C(=O)NC5)nc3CNC(=O)[C@H]2C1 ; CN1CCN2c3ccc(Nc4cnc(-c5ccnc6c5ccn6C)c5c4C(=O)NC5)nc3CNC[C@@H]2C1 ; CN1CCN2c3ccc(Nc4cnc(-c5ccnc6c5ccn6C)c5c4C(=O)NC5)nc3CNC[C@H]2C1]

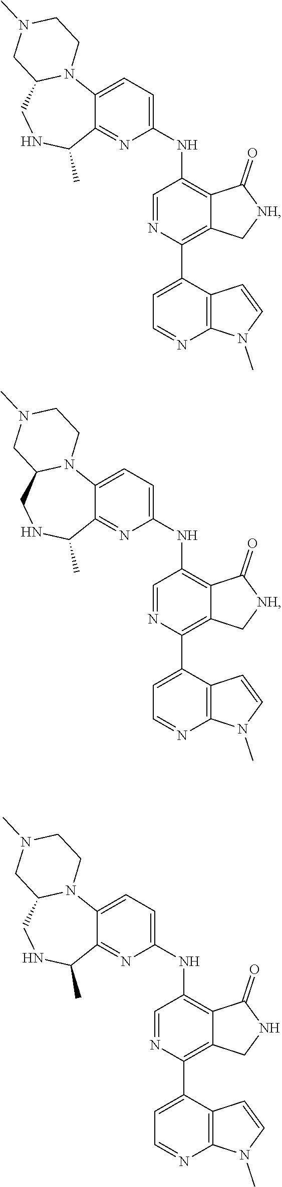 [SMILES: C[C@@H]1NC[C@@H]2CN(C)CCN2c2ccc(Nc3cnc(-c4ccnc5c4ccn5C)c4c3C(=O)NC4)nc21 ; C[C@@H]1NC[C@H]2CN(C)CCN2c2ccc(Nc3cnc(-c4ccnc5c4ccn5C)c4c3C(=O)NC4)nc21 ; C[C@H]1NC[C@@H]2CN(C)CCN2c2ccc(Nc3cnc(-c4ccnc5c4ccn5C)c4c3C(=O)NC4)nc21]

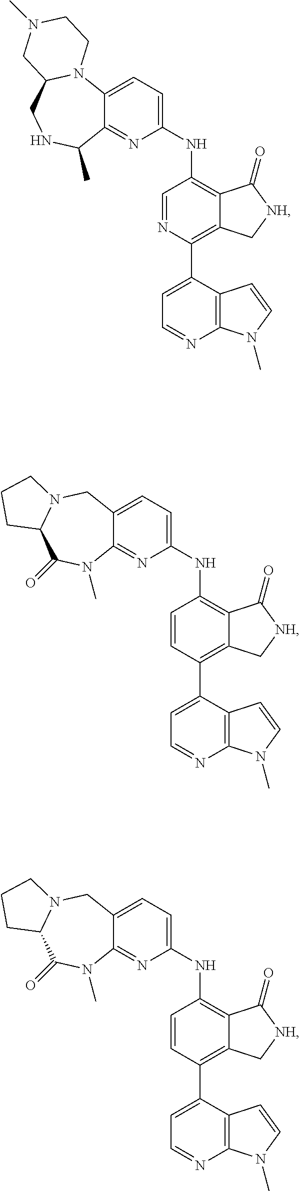 [SMILES: C[C@H]1NC[C@H]2CN(C)CCN2c2ccc(Nc3cnc(-c4ccnc5c4ccn5C)c4c3C(=O)NC4)nc21 ; CN1C(=O)[C@H]2CCCN2Cc2ccc(Nc3ccc(-c4ccnc5c4ccn5C)c4c3C(=O)NC4)nc21 ; CN1C(=O)[C@@H]2CCCN2Cc2ccc(Nc3ccc(-c4ccnc5c4ccn5C)c4c3C(=O)NC4)nc21]

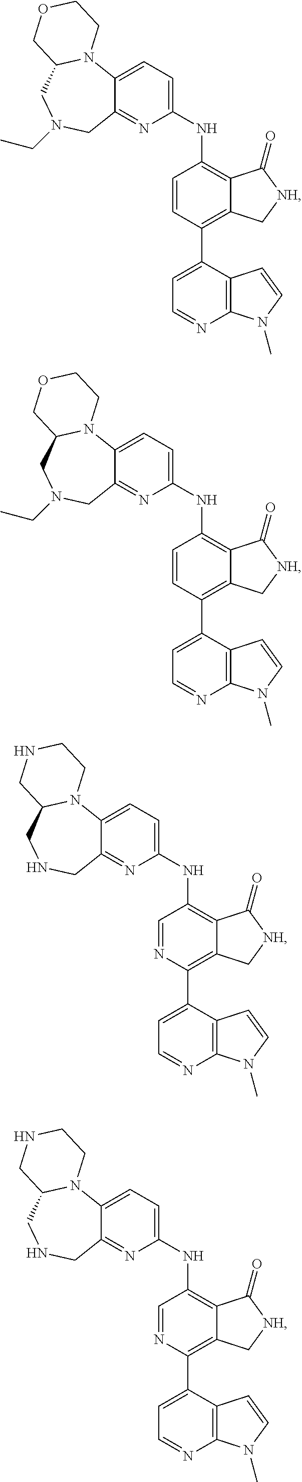 [SMILES: CCN1Cc2nc(Nc3ccc(-c4ccnc5c4ccn5C)c4c3C(=O)NC4)ccc2N2CCOC[C@H]2C1 ; CCN1Cc2nc(Nc3ccc(-c4ccnc5c4ccn5C)c4c3C(=O)NC4)ccc2N2CCOC[C@@H]2C1 ; Cn1ccc2c(-c3ncc(Nc4ccc5c(n4)CNC[C@H]4CNCCN54)c4c3CNC4=O)ccnc21 ; Cn1ccc2c(-c3ncc(Nc4ccc5c(n4)CNC[C@@H]4CNCCN54)c4c3CNC4=O)ccnc21]

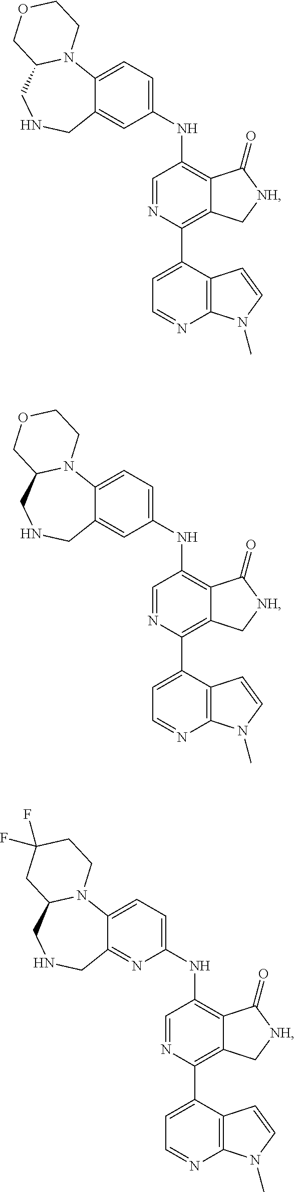 [SMILES: Cn1ccc2c(-c3ncc(Nc4ccc5c(c4)CNC[C@@H]4COCCN54)c4c3CNC4=O)ccnc21 ; Cn1ccc2c(-c3ncc(Nc4ccc5c(c4)CNC[C@H]4COCCN54)c4c3CNC4=O)ccnc21 ; Cn1ccc2c(-c3ncc(Nc4ccc5c(n4)CNC[C@H]4CC(F)(F)CCN54)c4c3CNC4=O)ccnc21]

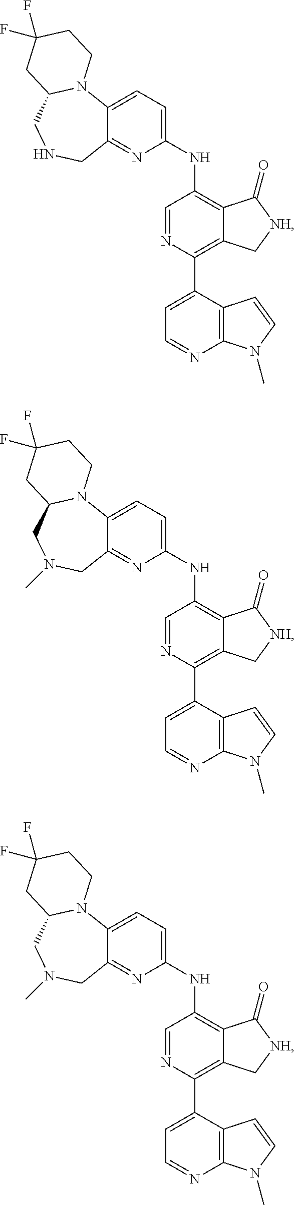 [SMILES: Cn1ccc2c(-c3ncc(Nc4ccc5c(n4)CNC[C@@H]4CC(F)(F)CCN54)c4c3CNC4=O)ccnc21 ; CN1Cc2nc(Nc3cnc(-c4ccnc5c4ccn5C)c4c3C(=O)NC4)ccc2N2CCC(F)(F)C[C@@H]2C1 ; CN1Cc2nc(Nc3cnc(-c4ccnc5c4ccn5C)c4c3C(=O)NC4)ccc2N2CCC(F)(F)C[C@H]2C1]

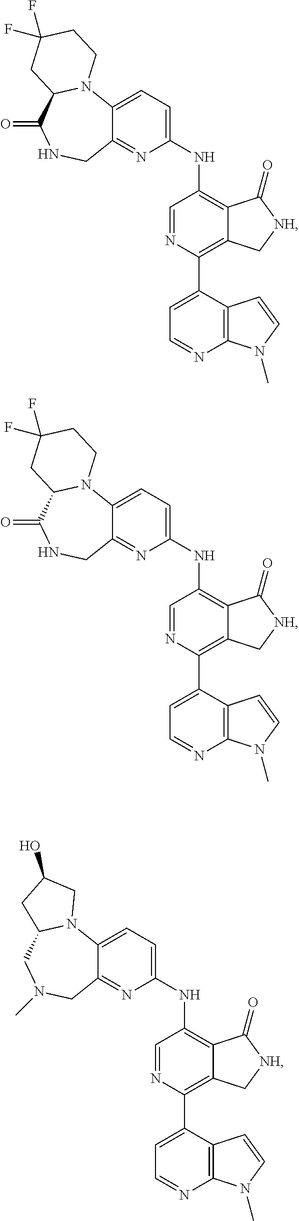 [SMILES: Cn1ccc2c(-c3ncc(Nc4ccc5c(n4)CNC(=O)[C@H]4CC(F)(F)CCN54)c4c3CNC4=O)ccnc21 ; Cn1ccc2c(-c3ncc(Nc4ccc5c(n4)CNC(=O)[C@@H]4CC(F)(F)CCN54)c4c3CNC4=O)ccnc21 ; CN1Cc2nc(Nc3cnc(-c4ccnc5c4ccn5C)c4c3C(=O)NC4)ccc2N2C[C@H](O)C[C@H]2C1]

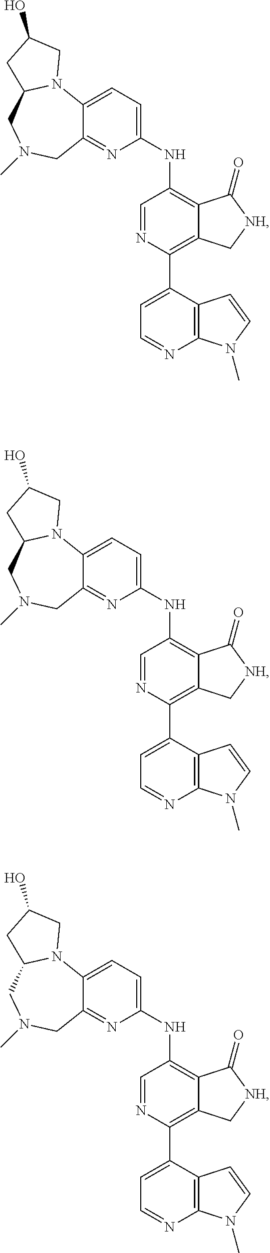 [SMILES: CN1Cc2nc(Nc3cnc(-c4ccnc5c4ccn5C)c4c3C(=O)NC4)ccc2N2C[C@H](O)C[C@@H]2C1 ; CN1Cc2nc(Nc3cnc(-c4ccnc5c4ccn5C)c4c3C(=O)NC4)ccc2N2C[C@@H](O)C[C@@H]2C1 ; CN1Cc2nc(Nc3cnc(-c4ccnc5c4ccn5C)c4c3C(=O)NC4)ccc2N2C[C@@H](O)C[C@H]2C1]

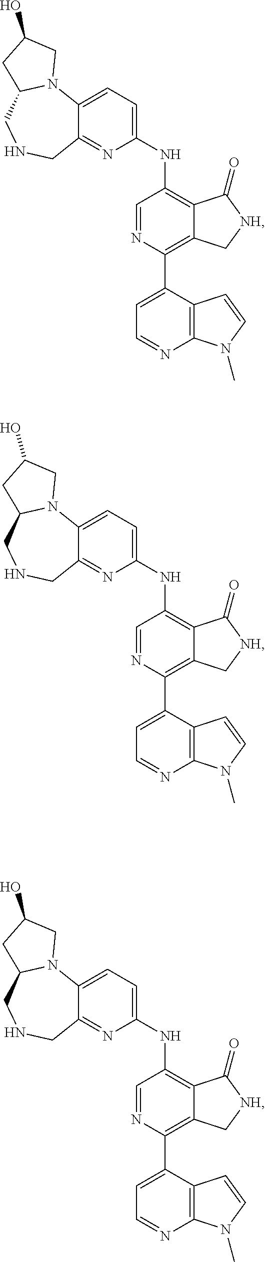 [SMILES: Cn1ccc2c(-c3ncc(Nc4ccc5c(n4)CNC[C@@H]4C[C@@H](O)CN54)c4c3CNC4=O)ccnc21 ; Cn1ccc2c(-c3ncc(Nc4ccc5c(n4)CNC[C@H]4C[C@H](O)CN54)c4c3CNC4=O)ccnc21 ; Cn1ccc2c(-c3ncc(Nc4ccc5c(n4)CNC[C@H]4C[C@@H](O)CN54)c4c3CNC4=O)ccnc21]

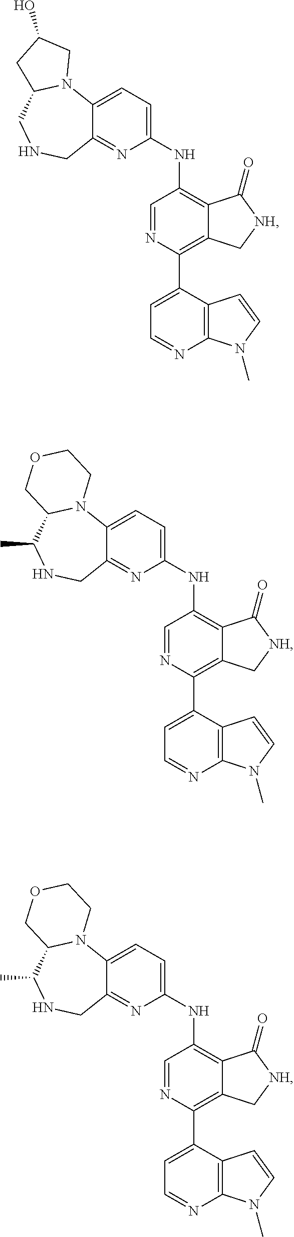 [SMILES: Cn1ccc2c(-c3ncc(Nc4ccc5c(n4)CNC[C@@H]4C[C@H](O)CN54)c4c3CNC4=O)ccnc21 ; C[C@@H]1NCc2nc(Nc3cnc(-c4ccnc5c4ccn5C)c4c3C(=O)NC4)ccc2N2CCOC[C@@H]12 ; C[C@H]1NCc2nc(Nc3cnc(-c4ccnc5c4ccn5C)c4c3C(=O)NC4)ccc2N2CCOC[C@@H]12]

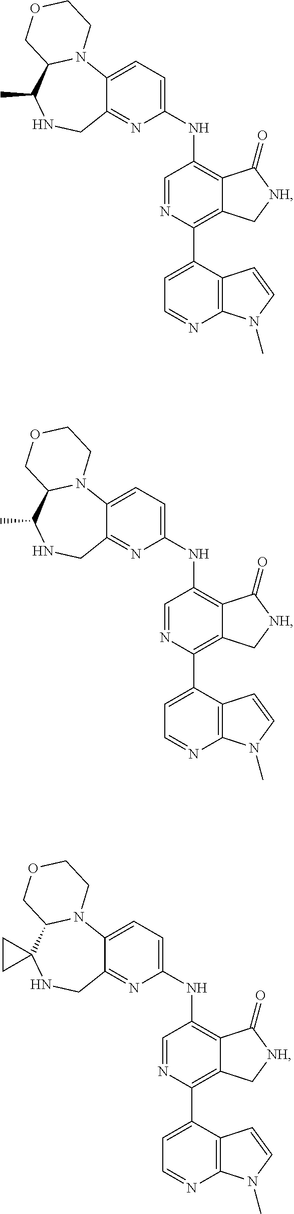 [SMILES: C[C@@H]1NCc2nc(Nc3cnc(-c4ccnc5c4ccn5C)c4c3C(=O)NC4)ccc2N2CCOC[C@H]12 ; C[C@H]1NCc2nc(Nc3cnc(-c4ccnc5c4ccn5C)c4c3C(=O)NC4)ccc2N2CCOC[C@H]12 ; Cn1ccc2c(-c3ncc(Nc4ccc5c(n4)CNC4(CC4)[C@@H]4COCCN54)c4c3CNC4=O)ccnc21]

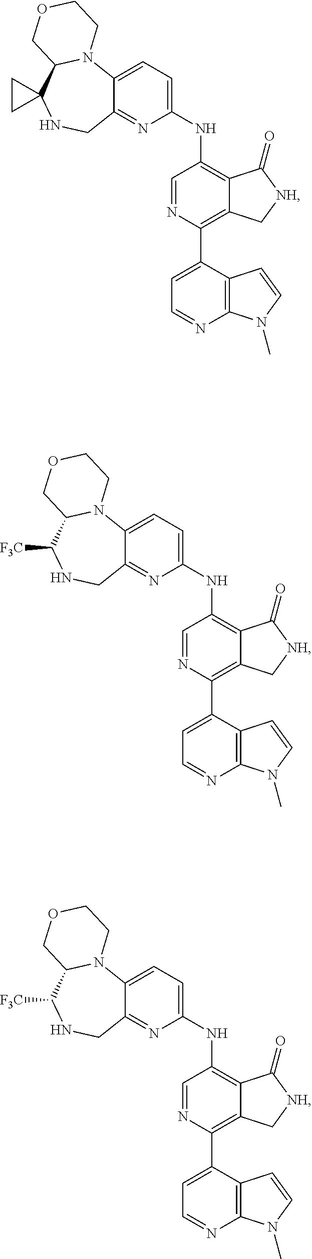 [SMILES: Cn1ccc2c(-c3ncc(Nc4ccc5c(n4)CNC4(CC4)[C@H]4COCCN54)c4c3CNC4=O)ccnc21 ; Cn1ccc2c(-c3ncc(Nc4ccc5c(n4)CN[C@@H](C(F)(F)F)[C@@H]4COCCN54)c4c3CNC4=O)ccnc21 ; Cn1ccc2c(-c3ncc(Nc4ccc5c(n4)CN[C@H](C(F)(F)F)[C@@H]4COCCN54)c4c3CNC4=O)ccnc21]

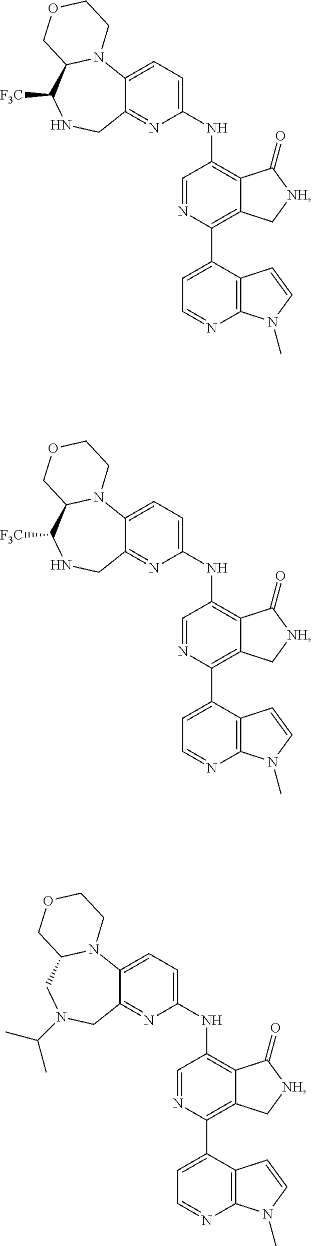 [SMILES: Cn1ccc2c(-c3ncc(Nc4ccc5c(n4)CN[C@@H](C(F)(F)F)[C@H]4COCCN54)c4c3CNC4=O)ccnc21 ; Cn1ccc2c(-c3ncc(Nc4ccc5c(n4)CN[C@H](C(F)(F)F)[C@H]4COCCN54)c4c3CNC4=O)ccnc21 ; CC(C)N1Cc2nc(Nc3cnc(-c4ccnc5c4ccn5C)c4c3C(=O)NC4)ccc2N2CCOC[C@H]2C1]

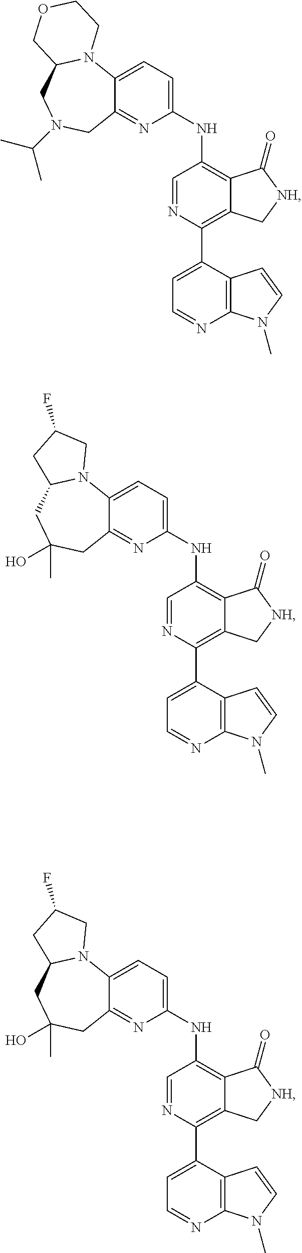

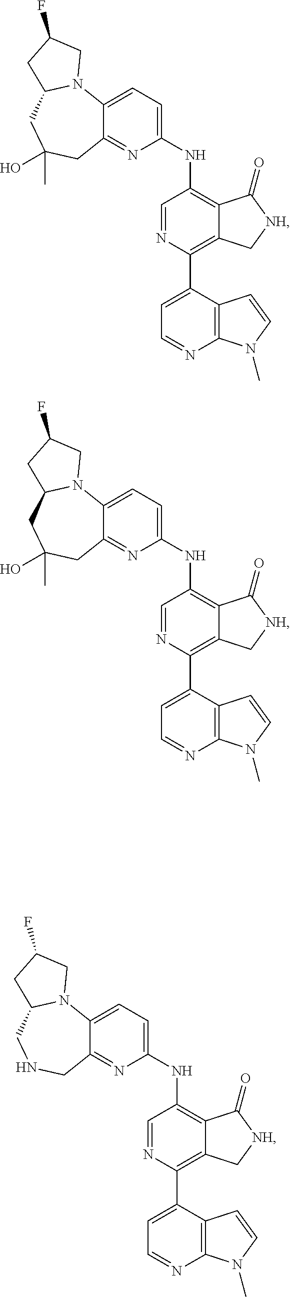

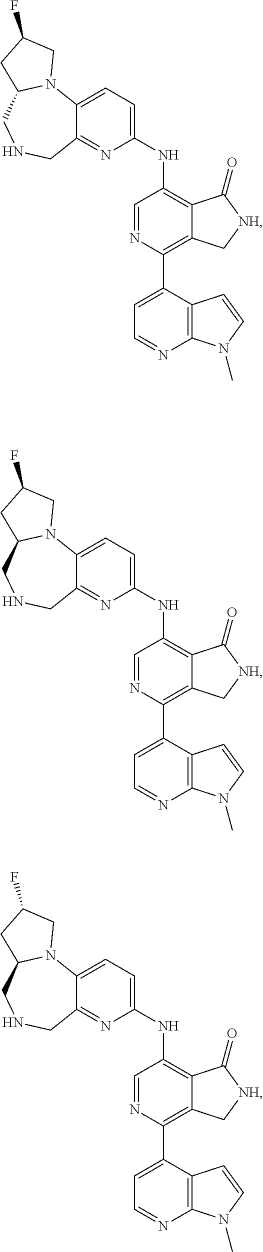 [SMILES: Cn1ccc2c(-c3ncc(Nc4ccc5c(n4)CNC[C@@H]4C[C@@H](F)CN54)c4c3CNC4=O)ccnc21 ; Cn1ccc2c(-c3ncc(Nc4ccc5c(n4)CNC[C@H]4C[C@@H](F)CN54)c4c3CNC4=O)ccnc21 ; Cn1ccc2c(-c3ncc(Nc4ccc5c(n4)CNC[C@H]4C[C@H](F)CN54)c4c3CNC4=O)ccnc21]

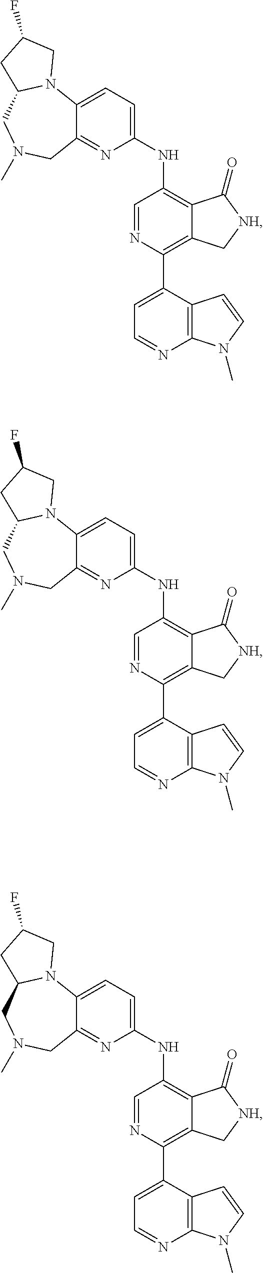 [SMILES: CN1Cc2nc(Nc3cnc(-c4ccnc5c4ccn5C)c4c3C(=O)NC4)ccc2N2C[C@@H](F)C[C@H]2C1 ; CN1Cc2nc(Nc3cnc(-c4ccnc5c4ccn5C)c4c3C(=O)NC4)ccc2N2C[C@H](F)C[C@H]2C1 ; CN1Cc2nc(Nc3cnc(-c4ccnc5c4ccn5C)c4c3C(=O)NC4)ccc2N2C[C@@H](F)C[C@@H]2C1]

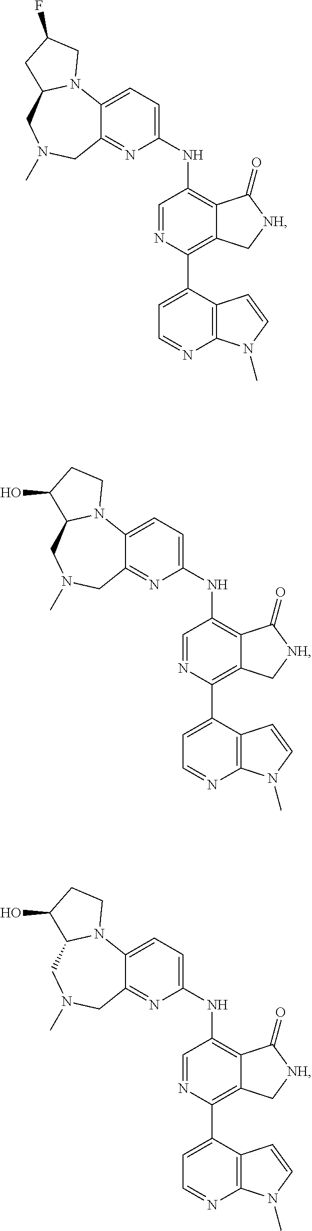 [SMILES: CN1Cc2nc(Nc3cnc(-c4ccnc5c4ccn5C)c4c3C(=O)NC4)ccc2N2C[C@H](F)C[C@@H]2C1 ; CN1Cc2nc(Nc3cnc(-c4ccnc5c4ccn5C)c4c3C(=O)NC4)ccc2N2CC[C@H](O)[C@@H]2C1 ; CN1Cc2nc(Nc3cnc(-c4ccnc5c4ccn5C)c4c3C(=O)NC4)ccc2N2CC[C@H](O)[C@H]2C1]

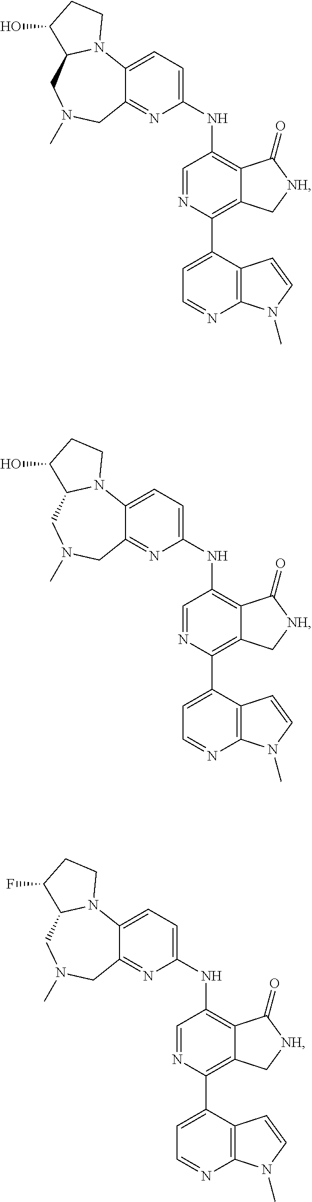 [SMILES: CN1Cc2nc(Nc3cnc(-c4ccnc5c4ccn5C)c4c3C(=O)NC4)ccc2N2CC[C@@H](O)[C@@H]2C1 ; CN1Cc2nc(Nc3cnc(-c4ccnc5c4ccn5C)c4c3C(=O)NC4)ccc2N2CC[C@@H](O)[C@H]2C1 ; CN1Cc2nc(Nc3cnc(-c4ccnc5c4ccn5C)c4c3C(=O)NC4)ccc2N2CC[C@@H](F)[C@H]2C1]

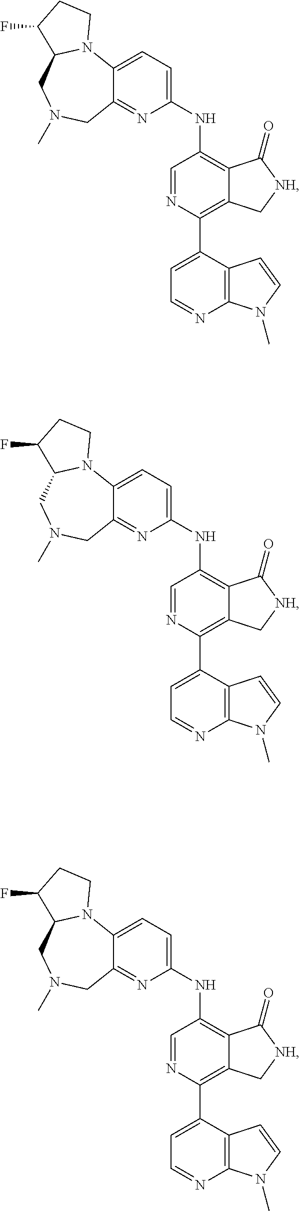 [SMILES: CN1Cc2nc(Nc3cnc(-c4ccnc5c4ccn5C)c4c3C(=O)NC4)ccc2N2CC[C@@H](F)[C@@H]2C1 ; CN1Cc2nc(Nc3cnc(-c4ccnc5c4ccn5C)c4c3C(=O)NC4)ccc2N2CC[C@H](F)[C@H]2C1 ; CN1Cc2nc(Nc3cnc(-c4ccnc5c4ccn5C)c4c3C(=O)NC4)ccc2N2CC[C@H](F)[C@@H]2C1]

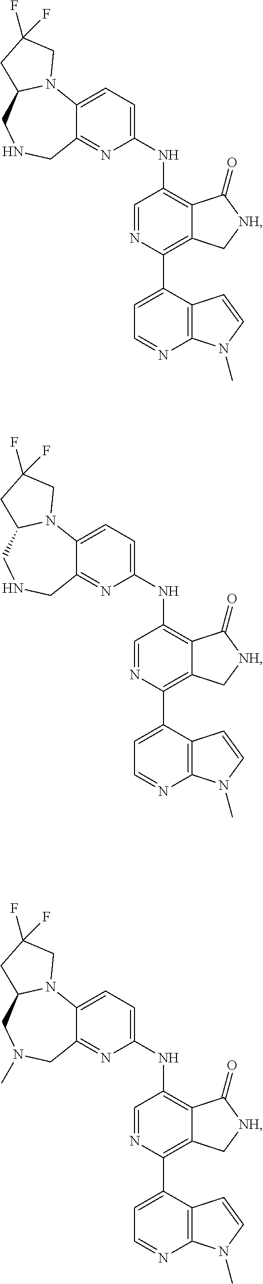 [SMILES: Cn1ccc2c(-c3ncc(Nc4ccc5c(n4)CNC[C@H]4CC(F)(F)CN54)c4c3CNC4=O)ccnc21 ; Cn1ccc2c(-c3ncc(Nc4ccc5c(n4)CNC[C@@H]4CC(F)(F)CN54)c4c3CNC4=O)ccnc21 ; CN1Cc2nc(Nc3cnc(-c4ccnc5c4ccn5C)c4c3C(=O)NC4)ccc2N2CC(F)(F)C[C@@H]2C1]

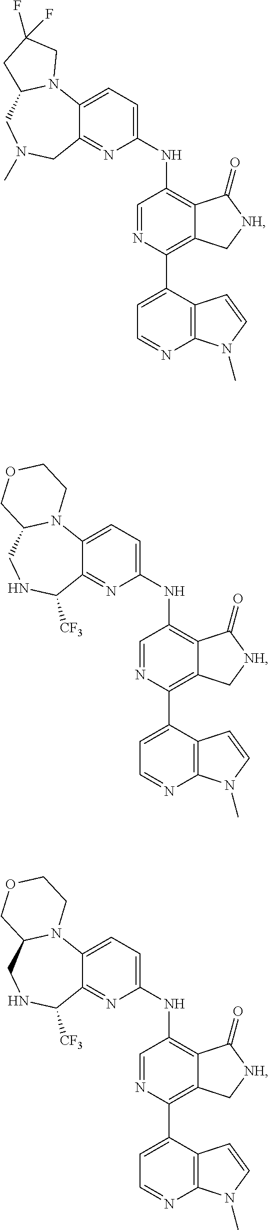 [SMILES: CN1Cc2nc(Nc3cnc(-c4ccnc5c4ccn5C)c4c3C(=O)NC4)ccc2N2CC(F)(F)C[C@H]2C1 ; Cn1ccc2c(-c3ncc(Nc4ccc5c(n4)[C@H](C(F)(F)F)NC[C@@H]4COCCN54)c4c3CNC4=O)ccnc21 ; Cn1ccc2c(-c3ncc(Nc4ccc5c(n4)[C@H](C(F)(F)F)NC[C@H]4COCCN54)c4c3CNC4=O)ccnc21]

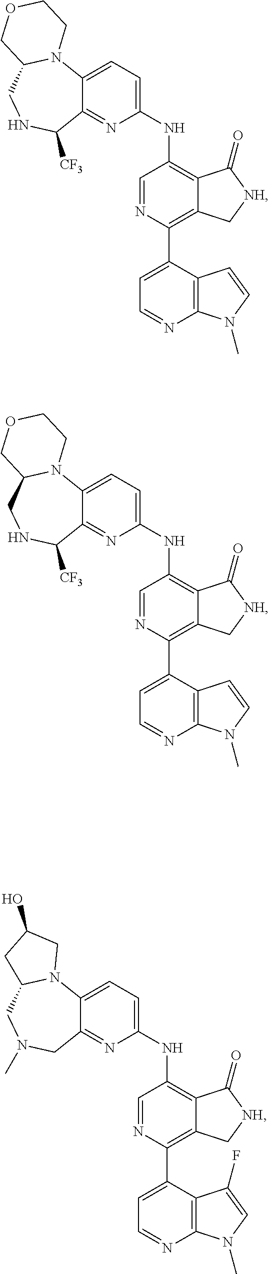 [SMILES: Cn1ccc2c(-c3ncc(Nc4ccc5c(n4)[C@@H](C(F)(F)F)NC[C@@H]4COCCN54)c4c3CNC4=O)ccnc21 ; Cn1ccc2c(-c3ncc(Nc4ccc5c(n4)[C@@H](C(F)(F)F)NC[C@H]4COCCN54)c4c3CNC4=O)ccnc21 ; CN1Cc2nc(Nc3cnc(-c4ccnc5c4c(F)cn5C)c4c3C(=O)NC4)ccc2N2C[C@H](O)C[C@H]2C1]

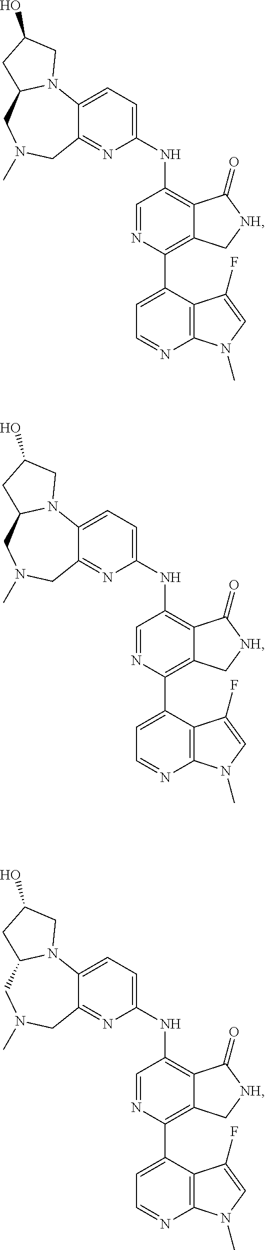 [SMILES: CN1Cc2nc(Nc3cnc(-c4ccnc5c4c(F)cn5C)c4c3C(=O)NC4)ccc2N2C[C@H](O)C[C@@H]2C1 ; CN1Cc2nc(Nc3cnc(-c4ccnc5c4c(F)cn5C)c4c3C(=O)NC4)ccc2N2C[C@@H](O)C[C@@H]2C1 ; CN1Cc2nc(Nc3cnc(-c4ccnc5c4c(F)cn5C)c4c3C(=O)NC4)ccc2N2C[C@@H](O)C[C@H]2C1]

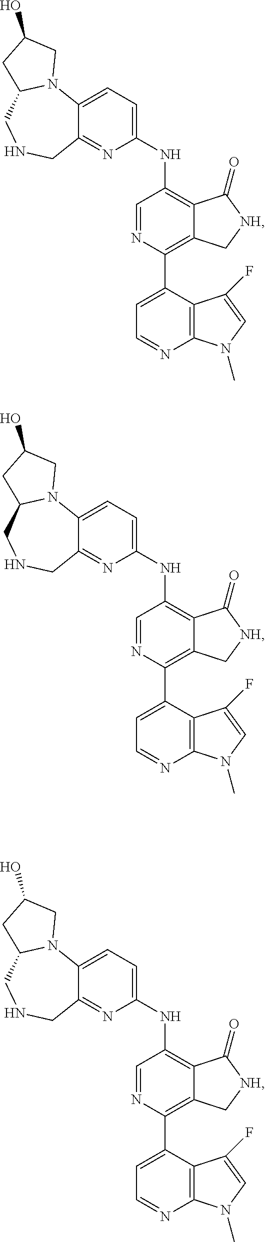 [SMILES: Cn1cc(F)c2c(-c3ncc(Nc4ccc5c(n4)CNC[C@@H]4C[C@@H](O)CN54)c4c3CNC4=O)ccnc21 ; Cn1cc(F)c2c(-c3ncc(Nc4ccc5c(n4)CNC[C@H]4C[C@@H](O)CN54)c4c3CNC4=O)ccnc21 ; Cn1cc(F)c2c(-c3ncc(Nc4ccc5c(n4)CNC[C@@H]4C[C@H](O)CN54)c4c3CNC4=O)ccnc21]

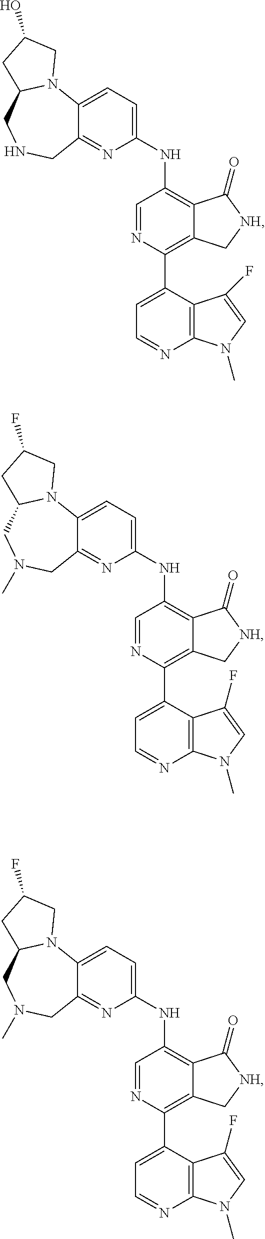 [SMILES: Cn1cc(F)c2c(-c3ncc(Nc4ccc5c(n4)CNC[C@H]4C[C@H](O)CN54)c4c3CNC4=O)ccnc21 ; CN1Cc2nc(Nc3cnc(-c4ccnc5c4c(F)cn5C)c4c3C(=O)NC4)ccc2N2C[C@@H](F)C[C@H]2C1 ; CN1Cc2nc(Nc3cnc(-c4ccnc5c4c(F)cn5C)c4c3C(=O)NC4)ccc2N2C[C@@H](F)C[C@@H]2C1]

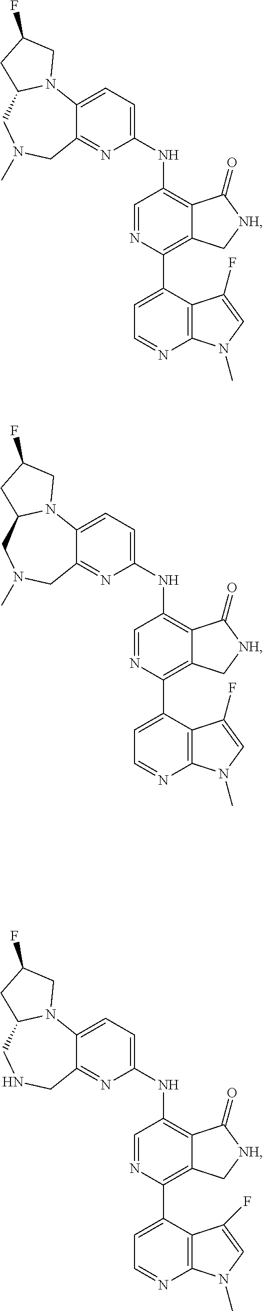 [SMILES: CN1Cc2nc(Nc3cnc(-c4ccnc5c4c(F)cn5C)c4c3C(=O)NC4)ccc2N2C[C@H](F)C[C@H]2C1 ; CN1Cc2nc(Nc3cnc(-c4ccnc5c4c(F)cn5C)c4c3C(=O)NC4)ccc2N2C[C@H](F)C[C@@H]2C1 ; Cn1cc(F)c2c(-c3ncc(Nc4ccc5c(n4)CNC[C@@H]4C[C@@H](F)CN54)c4c3CNC4=O)ccnc21]

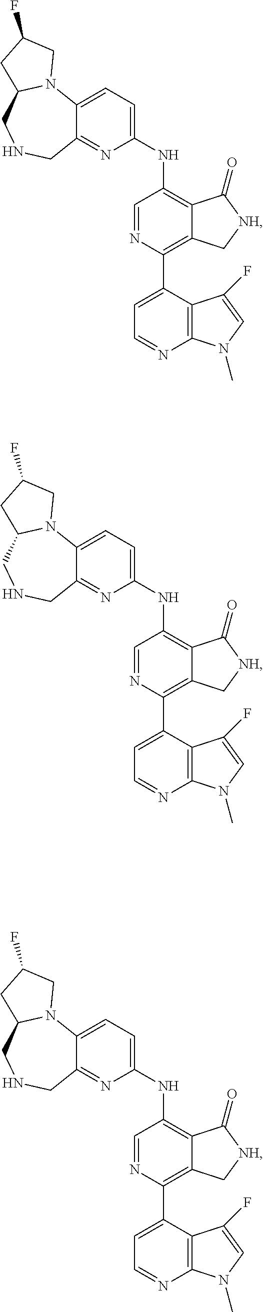 [SMILES: Cn1cc(F)c2c(-c3ncc(Nc4ccc5c(n4)CNC[C@H]4C[C@@H](F)CN54)c4c3CNC4=O)ccnc21 ; Cn1cc(F)c2c(-c3ncc(Nc4ccc5c(n4)CNC[C@@H]4C[C@H](F)CN54)c4c3CNC4=O)ccnc21 ; Cn1cc(F)c2c(-c3ncc(Nc4ccc5c(n4)CNC[C@H]4C[C@H](F)CN54)c4c3CNC4=O)ccnc21]

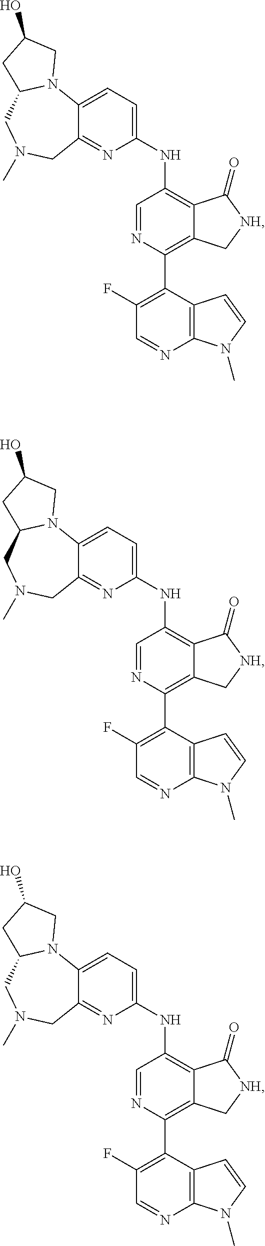 [SMILES: CN1Cc2nc(Nc3cnc(-c4c(F)cnc5c4ccn5C)c4c3C(=O)NC4)ccc2N2C[C@H](O)C[C@H]2C1 ; CN1Cc2nc(Nc3cnc(-c4c(F)cnc5c4ccn5C)c4c3C(=O)NC4)ccc2N2C[C@H](O)C[C@@H]2C1 ; CN1Cc2nc(Nc3cnc(-c4c(F)cnc5c4ccn5C)c4c3C(=O)NC4)ccc2N2C[C@@H](O)C[C@H]2C1]

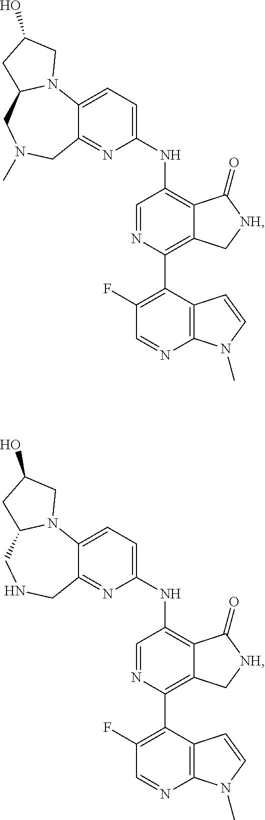 [SMILES: CN1Cc2nc(Nc3cnc(-c4c(F)cnc5c4ccn5C)c4c3C(=O)NC4)ccc2N2C[C@@H](O)C[C@@H]2C1 ; Cn1ccc2c(-c3ncc(Nc4ccc5c(n4)CNC[C@@H]4C[C@@H](O)CN54)c4c3CNC4=O)c(F)cnc21]

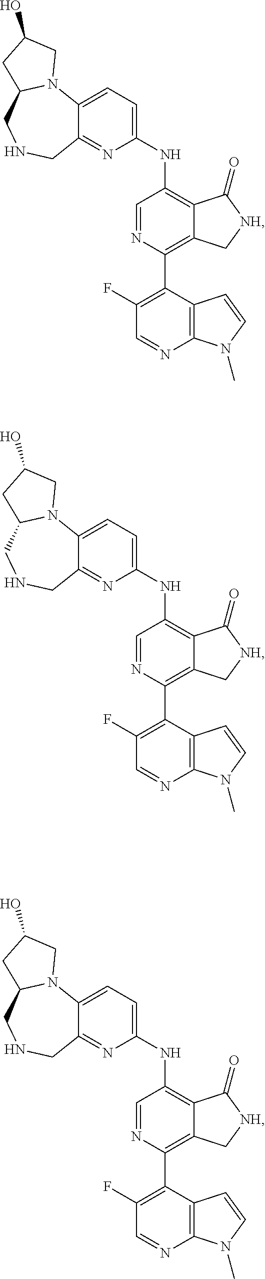 [SMILES: Cn1ccc2c(-c3ncc(Nc4ccc5c(n4)CNC[C@H]4C[C@@H](O)CN54)c4c3CNC4=O)c(F)cnc21 ; Cn1ccc2c(-c3ncc(Nc4ccc5c(n4)CNC[C@@H]4C[C@H](O)CN54)c4c3CNC4=O)c(F)cnc21 ; Cn1ccc2c(-c3ncc(Nc4ccc5c(n4)CNC[C@H]4C[C@H](O)CN54)c4c3CNC4=O)c(F)cnc21]

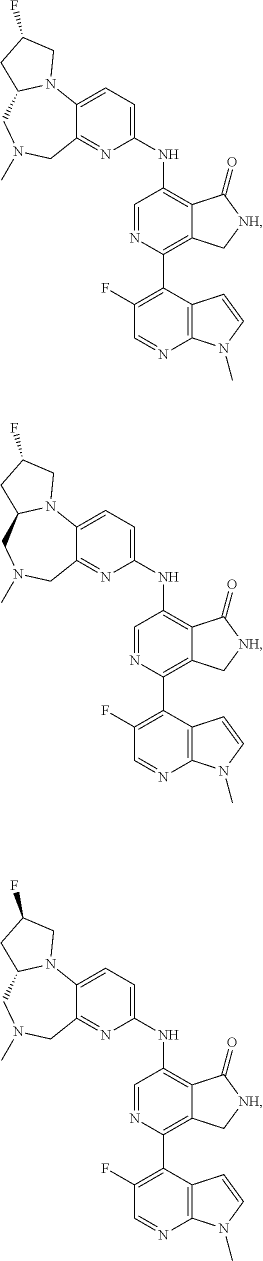 [SMILES: CN1Cc2nc(Nc3cnc(-c4c(F)cnc5c4ccn5C)c4c3C(=O)NC4)ccc2N2C[C@@H](F)C[C@H]2C1 ; CN1Cc2nc(Nc3cnc(-c4c(F)cnc5c4ccn5C)c4c3C(=O)NC4)ccc2N2C[C@@H](F)C[C@@H]2C1 ; CN1Cc2nc(Nc3cnc(-c4c(F)cnc5c4ccn5C)c4c3C(=O)NC4)ccc2N2C[C@H](F)C[C@H]2C1]

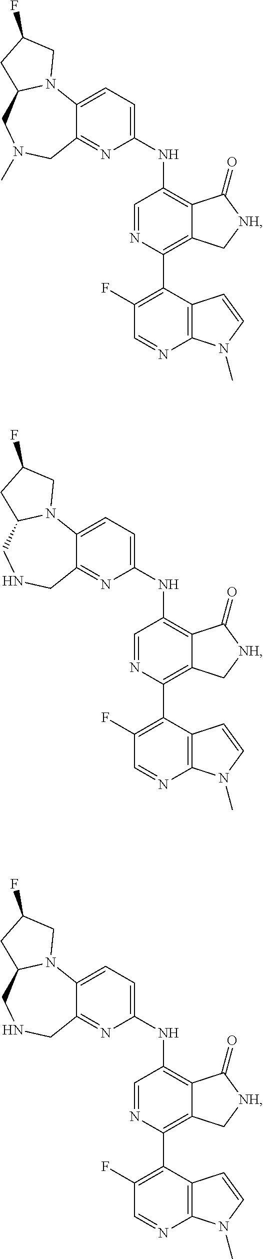 [SMILES: CN1Cc2nc(Nc3cnc(-c4c(F)cnc5c4ccn5C)c4c3C(=O)NC4)ccc2N2C[C@H](F)C[C@@H]2C1 ; Cn1ccc2c(-c3ncc(Nc4ccc5c(n4)CNC[C@@H]4C[C@@H](F)CN54)c4c3CNC4=O)c(F)cnc21 ; Cn1ccc2c(-c3ncc(Nc4ccc5c(n4)CNC[C@H]4C[C@@H](F)CN54)c4c3CNC4=O)c(F)cnc21]

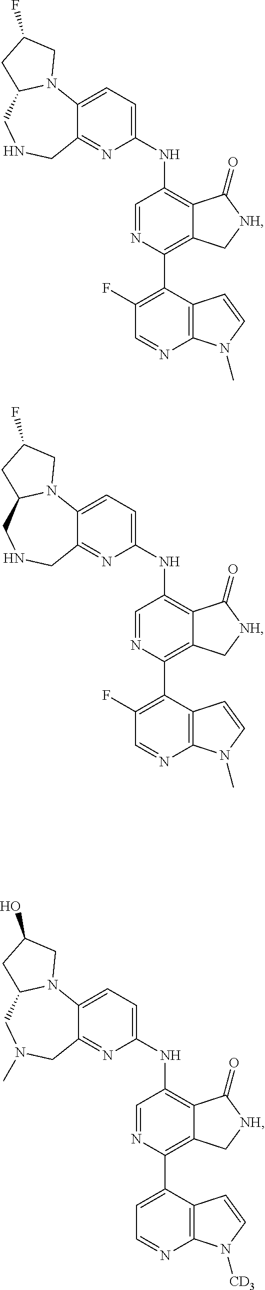 [SMILES: Cn1ccc2c(-c3ncc(Nc4ccc5c(n4)CNC[C@@H]4C[C@H](F)CN54)c4c3CNC4=O)c(F)cnc21 ; Cn1ccc2c(-c3ncc(Nc4ccc5c(n4)CNC[C@H]4C[C@H](F)CN54)c4c3CNC4=O)c(F)cnc21 ; [2H]C([2H])([2H])n1ccc2c(-c3ncc(Nc4ccc5c(n4)CN(C)C[C@@H]4C[C@@H](O)CN54)c4c3CNC4=O)ccnc21]

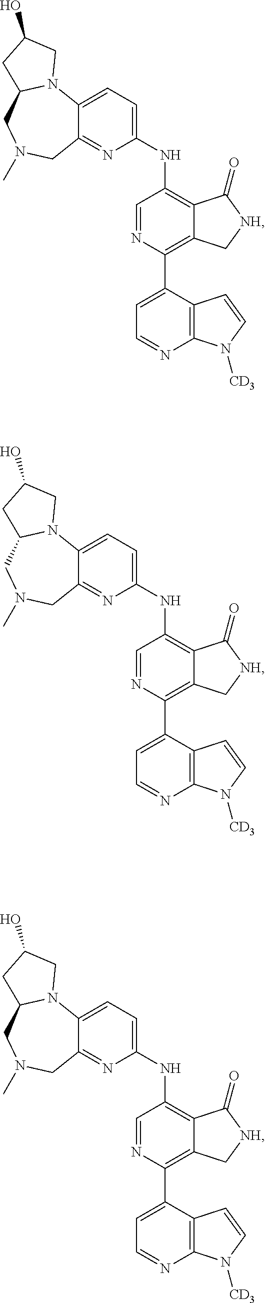 [SMILES: [2H]C([2H])([2H])n1ccc2c(-c3ncc(Nc4ccc5c(n4)CN(C)C[C@H]4C[C@@H](O)CN54)c4c3CNC4=O)ccnc21 ; [2H]C([2H])([2H])n1ccc2c(-c3ncc(Nc4ccc5c(n4)CN(C)C[C@@H]4C[C@H](O)CN54)c4c3CNC4=O)ccnc21 ; [2H]C([2H])([2H])n1ccc2c(-c3ncc(Nc4ccc5c(n4)CN(C)C[C@H]4C[C@H](O)CN54)c4c3CNC4=O)ccnc21]

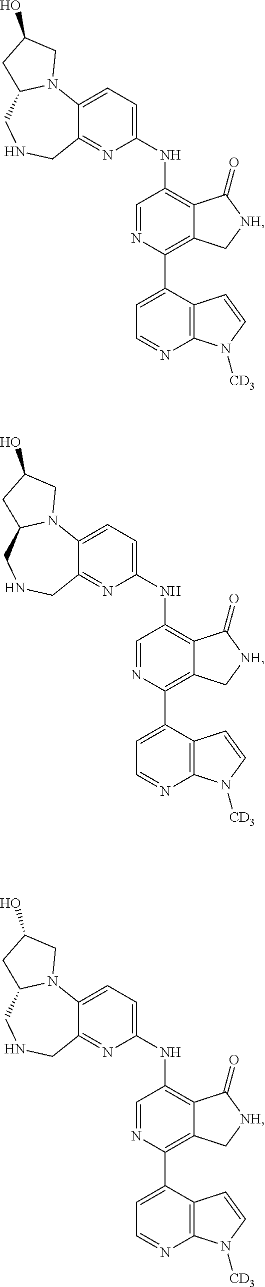 [SMILES: [2H]C([2H])([2H])n1ccc2c(-c3ncc(Nc4ccc5c(n4)CNC[C@@H]4C[C@@H](O)CN54)c4c3CNC4=O)ccnc21 ; [2H]C([2H])([2H])n1ccc2c(-c3ncc(Nc4ccc5c(n4)CNC[C@H]4C[C@@H](O)CN54)c4c3CNC4=O)ccnc21 ; [2H]C([2H])([2H])n1ccc2c(-c3ncc(Nc4ccc5c(n4)CNC[C@@H]4C[C@H](O)CN54)c4c3CNC4=O)ccnc21]

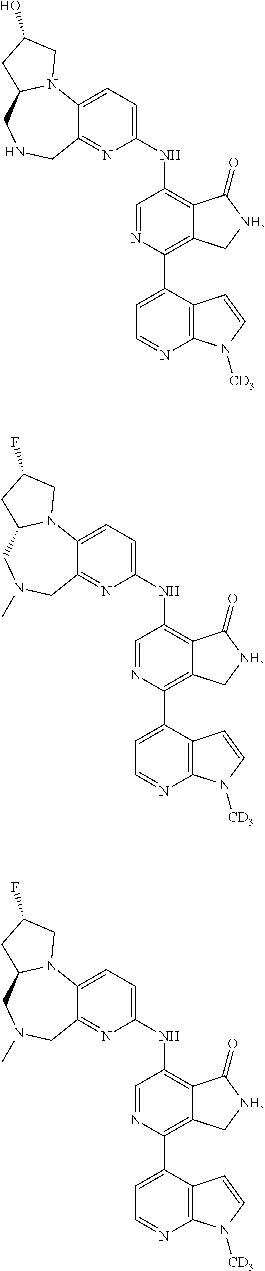 [SMILES: [2H]C([2H])([2H])n1ccc2c(-c3ncc(Nc4ccc5c(n4)CNC[C@H]4C[C@H](O)CN54)c4c3CNC4=O)ccnc21 ; [2H]C([2H])([2H])n1ccc2c(-c3ncc(Nc4ccc5c(n4)CN(C)C[C@@H]4C[C@H](F)CN54)c4c3CNC4=O)ccnc21 ; [2H]C([2H])([2H])n1ccc2c(-c3ncc(Nc4ccc5c(n4)CN(C)C[C@H]4C[C@H](F)CN54)c4c3CNC4=O)ccnc21]

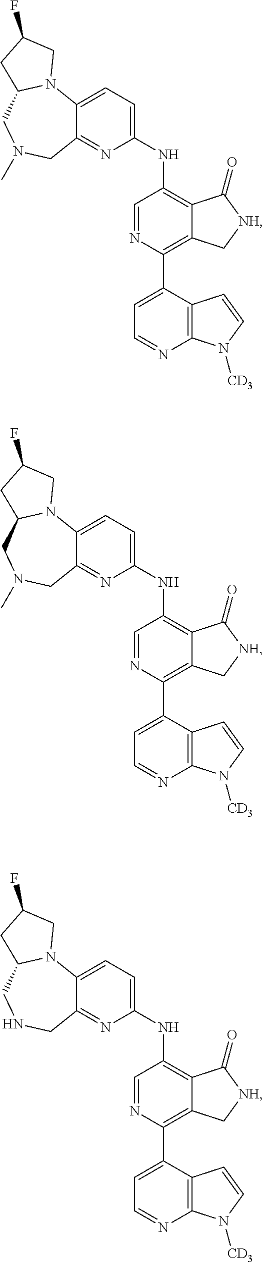 [SMILES: [2H]C([2H])([2H])n1ccc2c(-c3ncc(Nc4ccc5c(n4)CN(C)C[C@@H]4C[C@@H](F)CN54)c4c3CNC4=O)ccnc21 ; [2H]C([2H])([2H])n1ccc2c(-c3ncc(Nc4ccc5c(n4)CN(C)C[C@H]4C[C@@H](F)CN54)c4c3CNC4=O)ccnc21 ; [2H]C([2H])([2H])n1ccc2c(-c3ncc(Nc4ccc5c(n4)CNC[C@@H]4C[C@@H](F)CN54)c4c3CNC4=O)ccnc21]

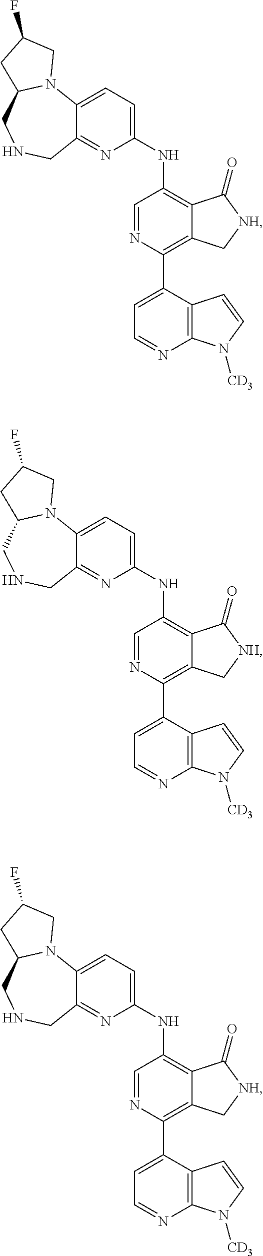 [SMILES: [2H]C([2H])([2H])n1ccc2c(-c3ncc(Nc4ccc5c(n4)CNC[C@H]4C[C@@H](F)CN54)c4c3CNC4=O)ccnc21 ; [2H]C([2H])([2H])n1ccc2c(-c3ncc(Nc4ccc5c(n4)CNC[C@@H]4C[C@H](F)CN54)c4c3CNC4=O)ccnc21 ; [2H]C([2H])([2H])n1ccc2c(-c3ncc(Nc4ccc5c(n4)CNC[C@H]4C[C@H](F)CN54)c4c3CNC4=O)ccnc21]

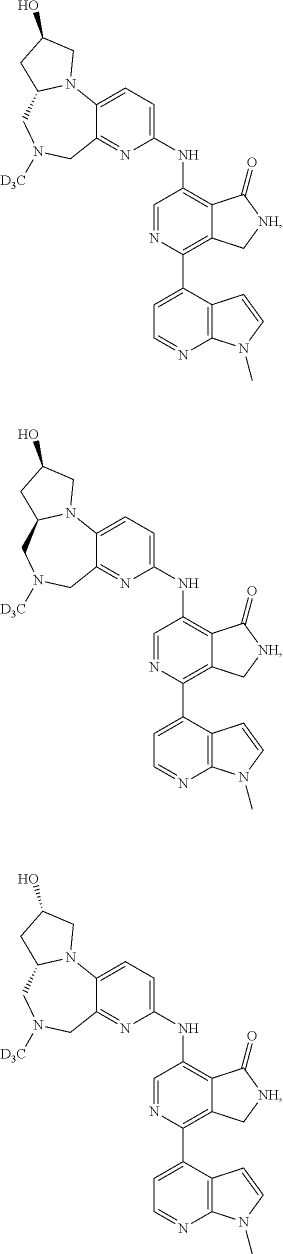 [SMILES: [2H]C([2H])([2H])N1Cc2nc(Nc3cnc(-c4ccnc5c4ccn5C)c4c3C(=O)NC4)ccc2N2C[C@H](O)C[C@H]2C1 ; [2H]C([2H])([2H])N1Cc2nc(Nc3cnc(-c4ccnc5c4ccn5C)c4c3C(=O)NC4)ccc2N2C[C@H](O)C[C@@H]2C1 ; [2H]C([2H])([2H])N1Cc2nc(Nc3cnc(-c4ccnc5c4ccn5C)c4c3C(=O)NC4)ccc2N2C[C@@H](O)C[C@H]2C1]

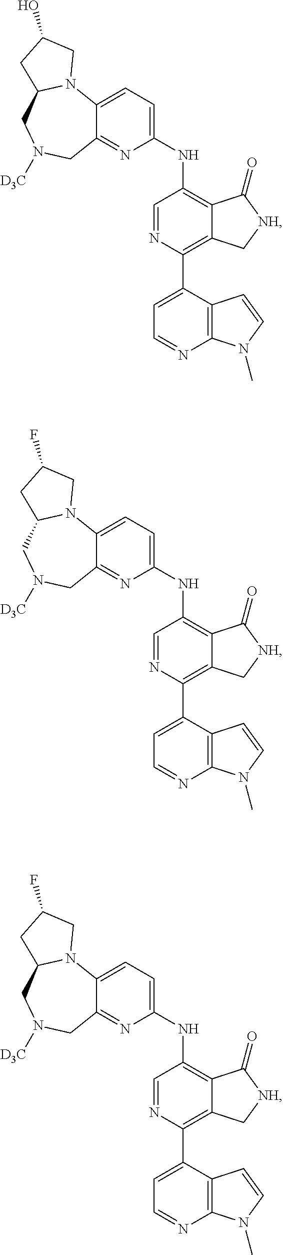 [SMILES: [2H]C([2H])([2H])N1Cc2nc(Nc3cnc(-c4ccnc5c4ccn5C)c4c3C(=O)NC4)ccc2N2C[C@@H](O)C[C@@H]2C1 ; [2H]C([2H])([2H])N1Cc2nc(Nc3cnc(-c4ccnc5c4ccn5C)c4c3C(=O)NC4)ccc2N2C[C@@H](F)C[C@H]2C1 ; [2H]C([2H])([2H])N1Cc2nc(Nc3cnc(-c4ccnc5c4ccn5C)c4c3C(=O)NC4)ccc2N2C[C@@H](F)C[C@@H]2C1]

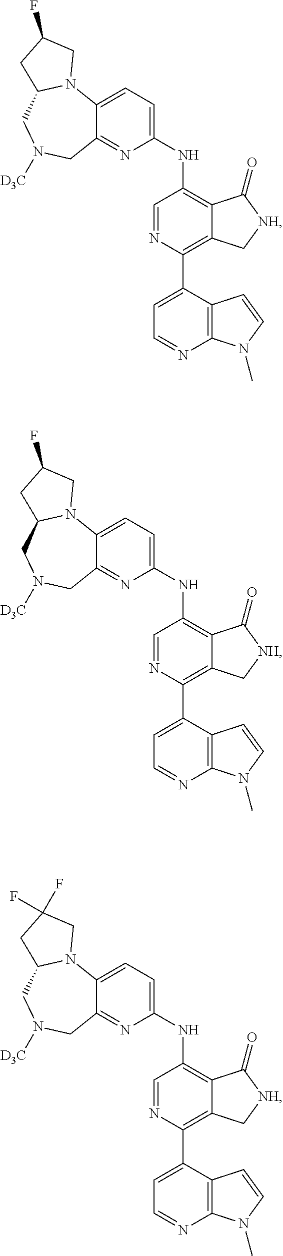 [SMILES: [2H]C([2H])([2H])N1Cc2nc(Nc3cnc(-c4ccnc5c4ccn5C)c4c3C(=O)NC4)ccc2N2C[C@H](F)C[C@H]2C1 ; [2H]C([2H])([2H])N1Cc2nc(Nc3cnc(-c4ccnc5c4ccn5C)c4c3C(=O)NC4)ccc2N2C[C@H](F)C[C@@H]2C1 ; [2H]C([2H])([2H])N1Cc2nc(Nc3cnc(-c4ccnc5c4ccn5C)c4c3C(=O)NC4)ccc2N2CC(F)(F)C[C@H]2C1]

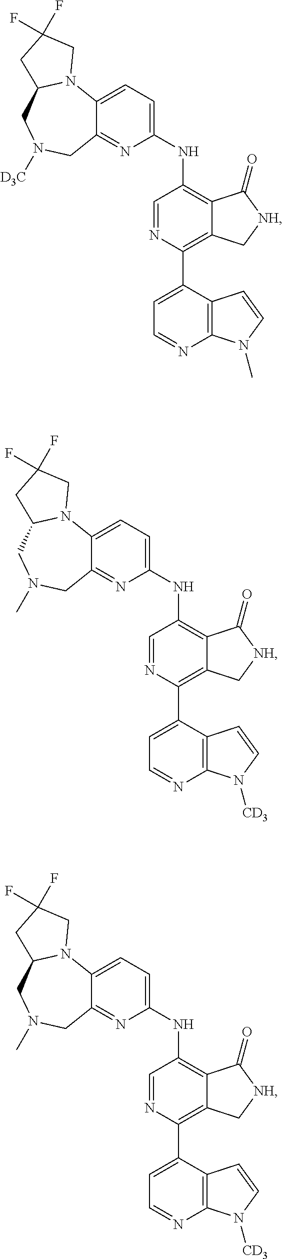 [SMILES: [2H]C([2H])([2H])N1Cc2nc(Nc3cnc(-c4ccnc5c4ccn5C)c4c3C(=O)NC4)ccc2N2CC(F)(F)C[C@@H]2C1 ; [2H]C([2H])([2H])n1ccc2c(-c3ncc(Nc4ccc5c(n4)CN(C)C[C@@H]4CC(F)(F)CN54)c4c3CNC4=O)ccnc21 ; [2H]C([2H])([2H])n1ccc2c(-c3ncc(Nc4ccc5c(n4)CN(C)C[C@H]4CC(F)(F)CN54)c4c3CNC4=O)ccnc21]

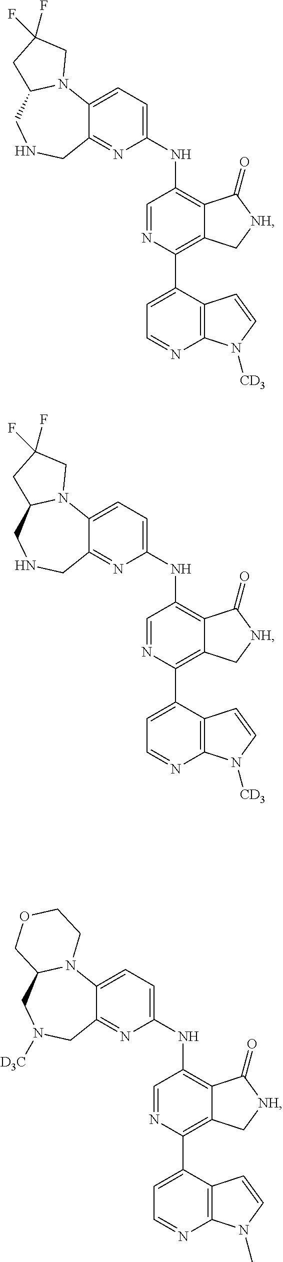 [SMILES: [2H]C([2H])([2H])n1ccc2c(-c3ncc(Nc4ccc5c(n4)CNC[C@@H]4CC(F)(F)CN54)c4c3CNC4=O)ccnc21 ; [2H]C([2H])([2H])n1ccc2c(-c3ncc(Nc4ccc5c(n4)CNC[C@H]4CC(F)(F)CN54)c4c3CNC4=O)ccnc21 ; [2H]C([2H])([2H])N1Cc2nc(Nc3cnc(-c4ccnc5c4ccn5C)c4c3C(=O)NC4)ccc2N2CCOC[C@@H]2C1]

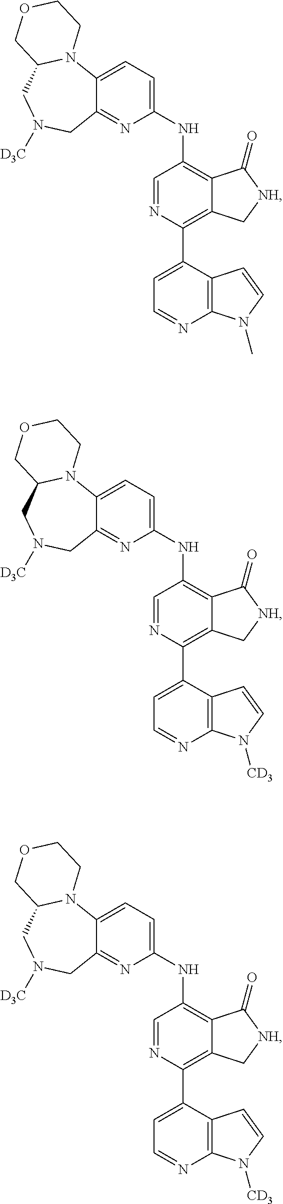 [SMILES: [2H]C([2H])([2H])N1Cc2nc(Nc3cnc(-c4ccnc5c4ccn5C)c4c3C(=O)NC4)ccc2N2CCOC[C@H]2C1 ; [2H]C([2H])([2H])N1Cc2nc(Nc3cnc(-c4ccnc5c4ccn5C([2H])([2H])[2H])c4c3C(=O)NC4)ccc2N2CCOC[C@@H]2C1 ; [2H]C([2H])([2H])N1Cc2nc(Nc3cnc(-c4ccnc5c4ccn5C([2H])([2H])[2H])c4c3C(=O)NC4)ccc2N2CCOC[C@H]2C1]

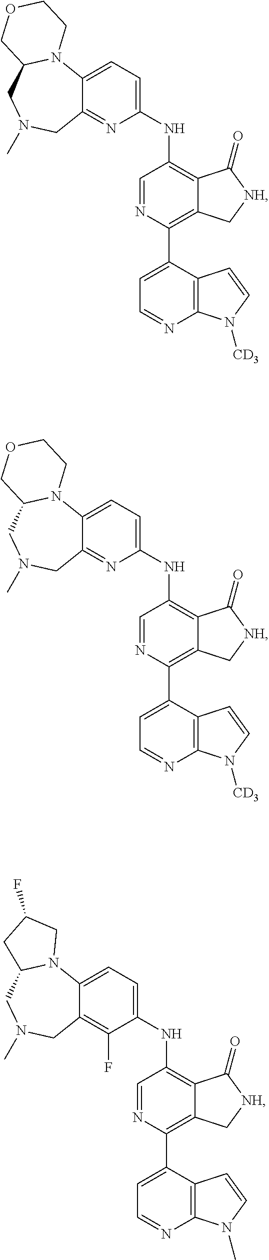 [SMILES: [2H]C([2H])([2H])n1ccc2c(-c3ncc(Nc4ccc5c(n4)CN(C)C[C@H]4COCCN54)c4c3CNC4=O)ccnc21 ; [2H]C([2H])([2H])n1ccc2c(-c3ncc(Nc4ccc5c(n4)CN(C)C[C@@H]4COCCN54)c4c3CNC4=O)ccnc21 ; CN1Cc2c(ccc(Nc3cnc(-c4ccnc5c4ccn5C)c4c3C(=O)NC4)c2F)N2C[C@@H](F)C[C@H]2C1]

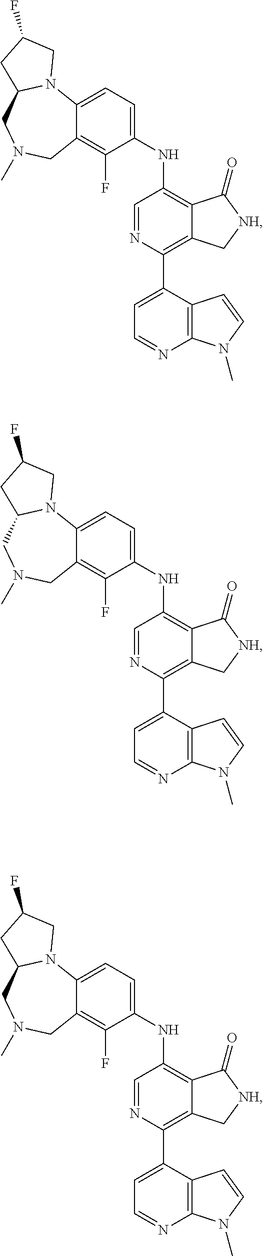 [SMILES: CN1Cc2c(ccc(Nc3cnc(-c4ccnc5c4ccn5C)c4c3C(=O)NC4)c2F)N2C[C@@H](F)C[C@@H]2C1 ; CN1Cc2c(ccc(Nc3cnc(-c4ccnc5c4ccn5C)c4c3C(=O)NC4)c2F)N2C[C@H](F)C[C@H]2C1 ; CN1Cc2c(ccc(Nc3cnc(-c4ccnc5c4ccn5C)c4c3C(=O)NC4)c2F)N2C[C@H](F)C[C@@H]2C1]

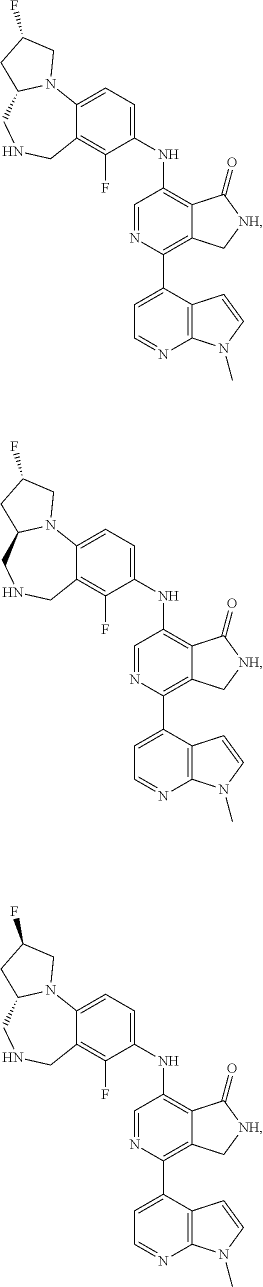 [SMILES: Cn1ccc2c(-c3ncc(Nc4ccc5c(c4F)CNC[C@@H]4C[C@H](F)CN54)c4c3CNC4=O)ccnc21 ; Cn1ccc2c(-c3ncc(Nc4ccc5c(c4F)CNC[C@H]4C[C@H](F)CN54)c4c3CNC4=O)ccnc21 ; Cn1ccc2c(-c3ncc(Nc4ccc5c(c4F)CNC[C@@H]4C[C@@H](F)CN54)c4c3CNC4=O)ccnc21]

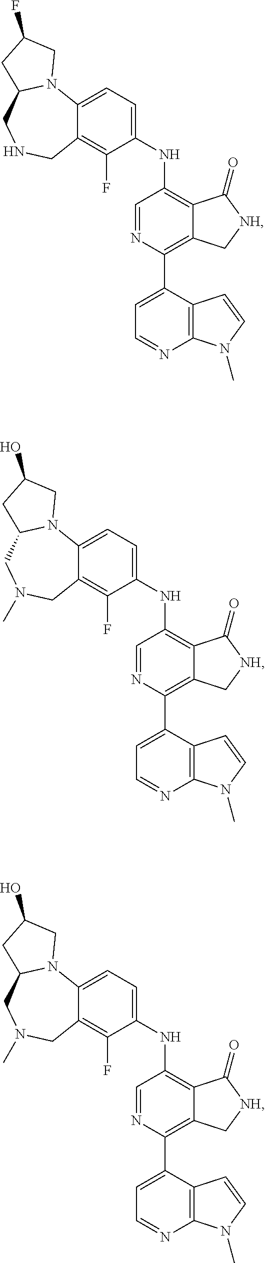 [SMILES: Cn1ccc2c(-c3ncc(Nc4ccc5c(c4F)CNC[C@H]4C[C@@H](F)CN54)c4c3CNC4=O)ccnc21 ; CN1Cc2c(ccc(Nc3cnc(-c4ccnc5c4ccn5C)c4c3C(=O)NC4)c2F)N2C[C@H](O)C[C@H]2C1 ; CN1Cc2c(ccc(Nc3cnc(-c4ccnc5c4ccn5C)c4c3C(=O)NC4)c2F)N2C[C@H](O)C[C@@H]2C1]

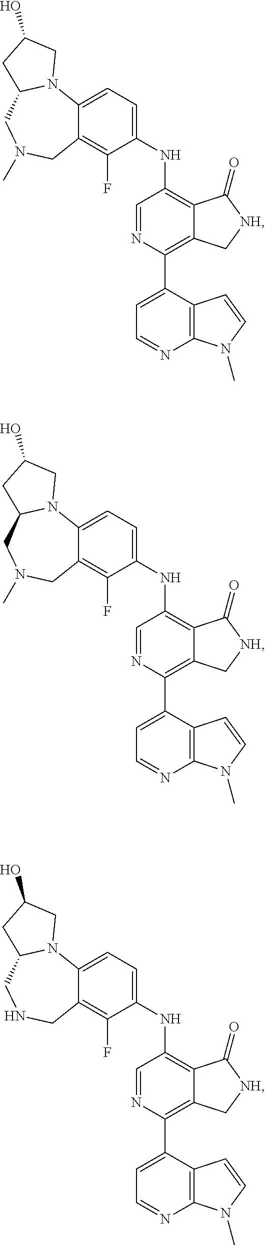 [SMILES: CN1Cc2c(ccc(Nc3cnc(-c4ccnc5c4ccn5C)c4c3C(=O)NC4)c2F)N2C[C@@H](O)C[C@H]2C1 ; CN1Cc2c(ccc(Nc3cnc(-c4ccnc5c4ccn5C)c4c3C(=O)NC4)c2F)N2C[C@@H](O)C[C@@H]2C1 ; Cn1ccc2c(-c3ncc(Nc4ccc5c(c4F)CNC[C@@H]4C[C@@H](O)CN54)c4c3CNC4=O)ccnc21]

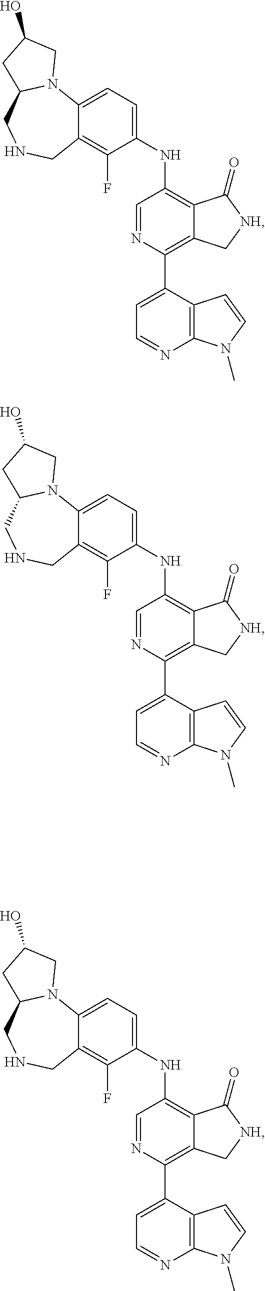 [SMILES: Cn1ccc2c(-c3ncc(Nc4ccc5c(c4F)CNC[C@H]4C[C@@H](O)CN54)c4c3CNC4=O)ccnc21 ; Cn1ccc2c(-c3ncc(Nc4ccc5c(c4F)CNC[C@@H]4C[C@H](O)CN54)c4c3CNC4=O)ccnc21 ; Cn1ccc2c(-c3ncc(Nc4ccc5c(c4F)CNC[C@H]4C[C@H](O)CN54)c4c3CNC4=O)ccnc21]

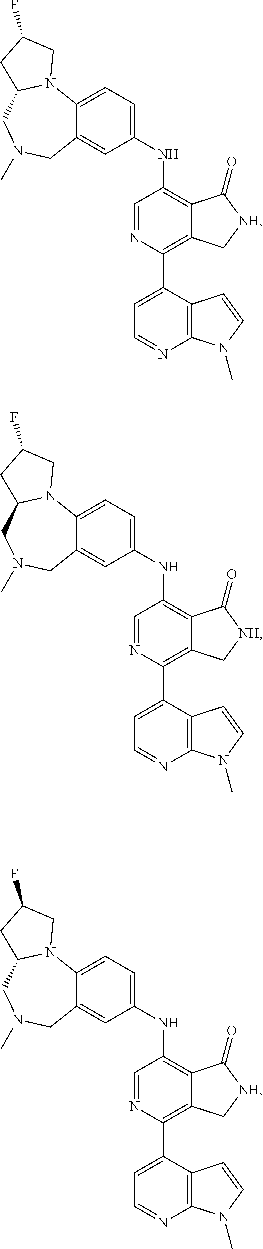 [SMILES: CN1Cc2cc(Nc3cnc(-c4ccnc5c4ccn5C)c4c3C(=O)NC4)ccc2N2C[C@@H](F)C[C@H]2C1 ; CN1Cc2cc(Nc3cnc(-c4ccnc5c4ccn5C)c4c3C(=O)NC4)ccc2N2C[C@@H](F)C[C@@H]2C1 ; CN1Cc2cc(Nc3cnc(-c4ccnc5c4ccn5C)c4c3C(=O)NC4)ccc2N2C[C@H](F)C[C@H]2C1]

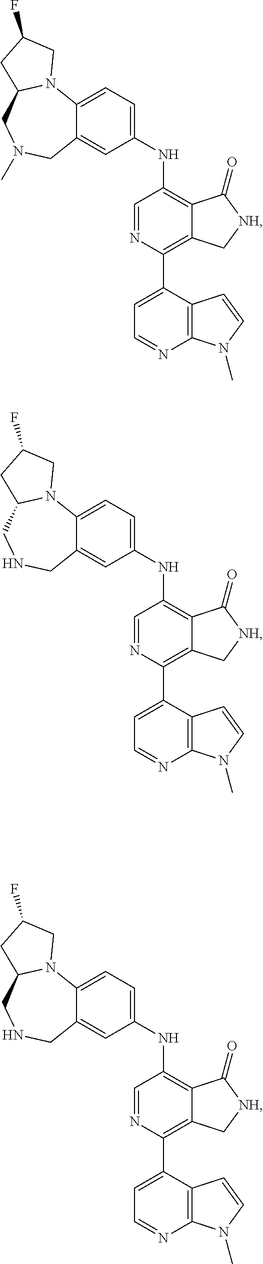 [SMILES: CN1Cc2cc(Nc3cnc(-c4ccnc5c4ccn5C)c4c3C(=O)NC4)ccc2N2C[C@H](F)C[C@@H]2C1 ; Cn1ccc2c(-c3ncc(Nc4ccc5c(c4)CNC[C@@H]4C[C@H](F)CN54)c4c3CNC4=O)ccnc21 ; Cn1ccc2c(-c3ncc(Nc4ccc5c(c4)CNC[C@H]4C[C@H](F)CN54)c4c3CNC4=O)ccnc21]

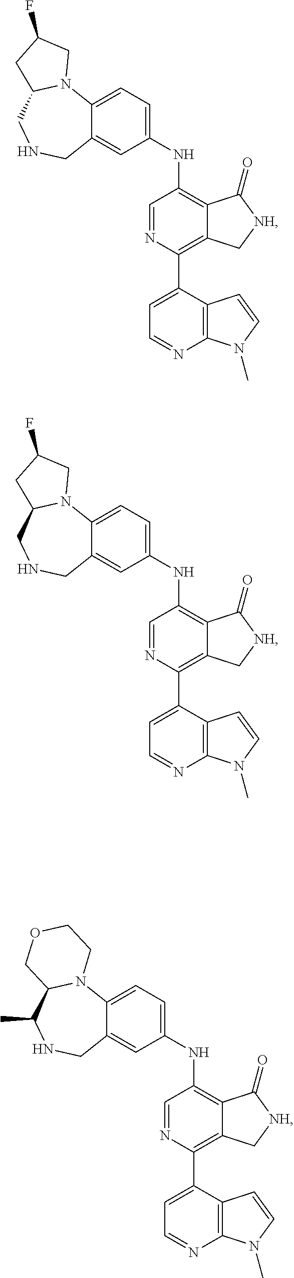 [SMILES: Cn1ccc2c(-c3ncc(Nc4ccc5c(c4)CNC[C@@H]4C[C@@H](F)CN54)c4c3CNC4=O)ccnc21 ; Cn1ccc2c(-c3ncc(Nc4ccc5c(c4)CNC[C@H]4C[C@@H](F)CN54)c4c3CNC4=O)ccnc21 ; C[C@@H]1NCc2cc(Nc3cnc(-c4ccnc5c4ccn5C)c4c3C(=O)NC4)ccc2N2CCOC[C@H]12]

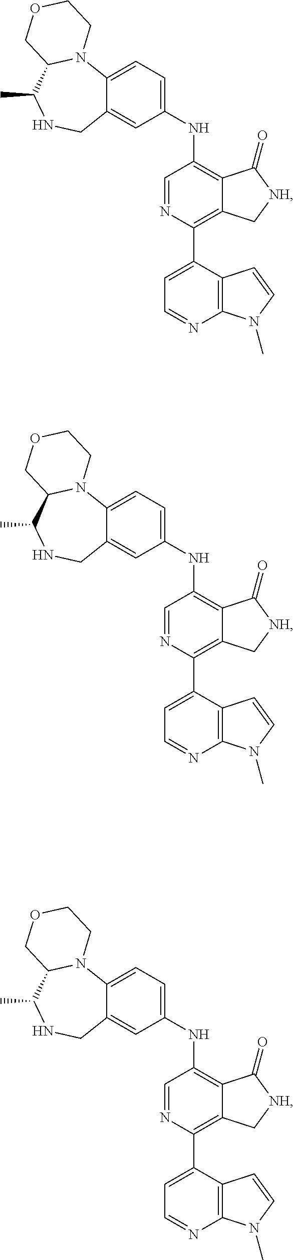 [SMILES: C[C@@H]1NCc2cc(Nc3cnc(-c4ccnc5c4ccn5C)c4c3C(=O)NC4)ccc2N2CCOC[C@@H]12 ; C[C@H]1NCc2cc(Nc3cnc(-c4ccnc5c4ccn5C)c4c3C(=O)NC4)ccc2N2CCOC[C@H]12 ; C[C@H]1NCc2cc(Nc3cnc(-c4ccnc5c4ccn5C)c4c3C(=O)NC4)ccc2N2CCOC[C@@H]12]

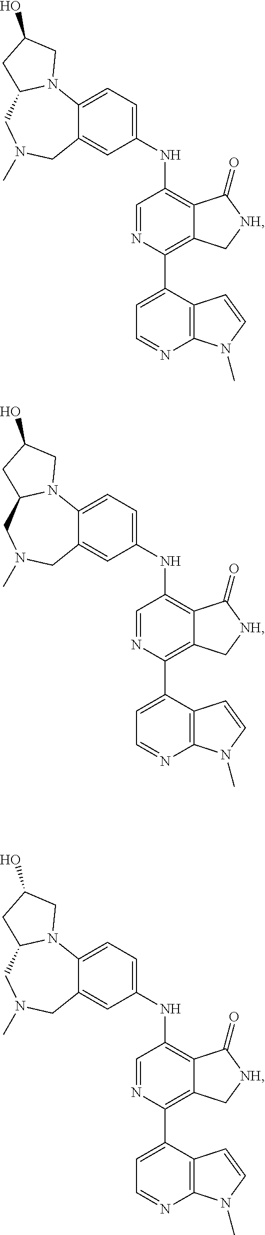 [SMILES: CN1Cc2cc(Nc3cnc(-c4ccnc5c4ccn5C)c4c3C(=O)NC4)ccc2N2C[C@H](O)C[C@H]2C1 ; CN1Cc2cc(Nc3cnc(-c4ccnc5c4ccn5C)c4c3C(=O)NC4)ccc2N2C[C@H](O)C[C@@H]2C1 ; CN1Cc2cc(Nc3cnc(-c4ccnc5c4ccn5C)c4c3C(=O)NC4)ccc2N2C[C@@H](O)C[C@H]2C1]

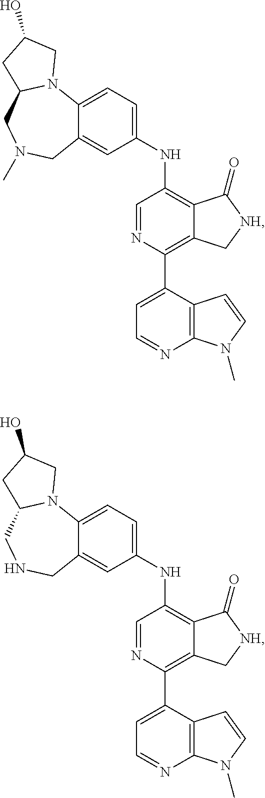 [SMILES: CN1Cc2cc(Nc3cnc(-c4ccnc5c4ccn5C)c4c3C(=O)NC4)ccc2N2C[C@@H](O)C[C@@H]2C1 ; Cn1ccc2c(-c3ncc(Nc4ccc5c(c4)CNC[C@@H]4C[C@@H](O)CN54)c4c3CNC4=O)ccnc21]

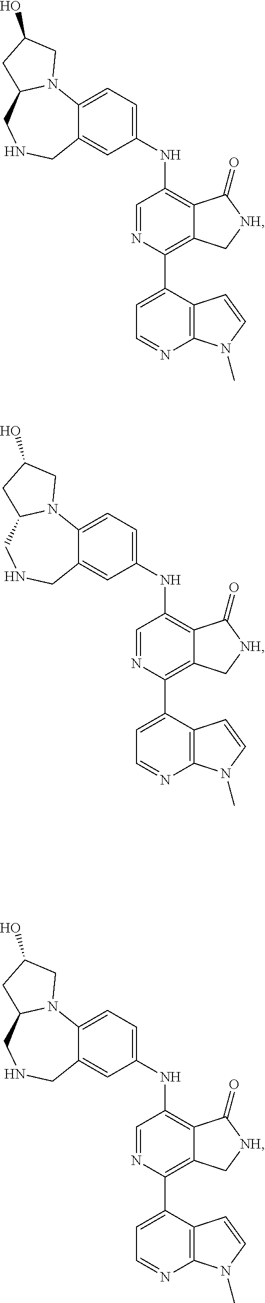 [SMILES: Cn1ccc2c(-c3ncc(Nc4ccc5c(c4)CNC[C@H]4C[C@@H](O)CN54)c4c3CNC4=O)ccnc21 ; Cn1ccc2c(-c3ncc(Nc4ccc5c(c4)CNC[C@@H]4C[C@H](O)CN54)c4c3CNC4=O)ccnc21 ; Cn1ccc2c(-c3ncc(Nc4ccc5c(c4)CNC[C@H]4C[C@H](O)CN54)c4c3CNC4=O)ccnc21]

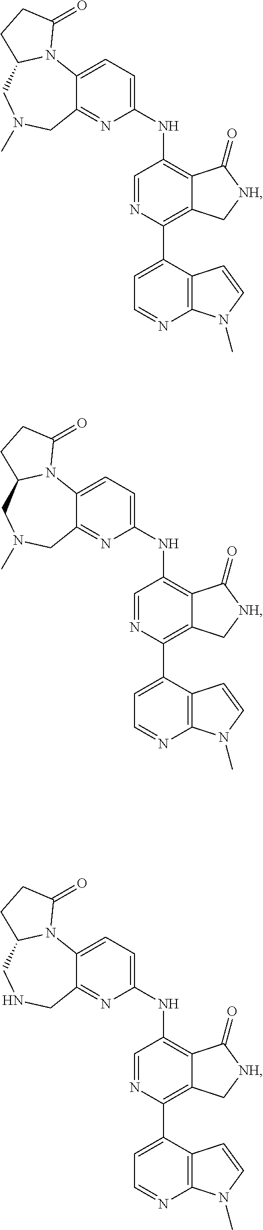 [SMILES: CN1Cc2nc(Nc3cnc(-c4ccnc5c4ccn5C)c4c3C(=O)NC4)ccc2N2C(=O)CC[C@H]2C1 ; CN1Cc2nc(Nc3cnc(-c4ccnc5c4ccn5C)c4c3C(=O)NC4)ccc2N2C(=O)CC[C@@H]2C1 ; Cn1ccc2c(-c3ncc(Nc4ccc5c(n4)CNC[C@@H]4CCC(=O)N54)c4c3CNC4=O)ccnc21]

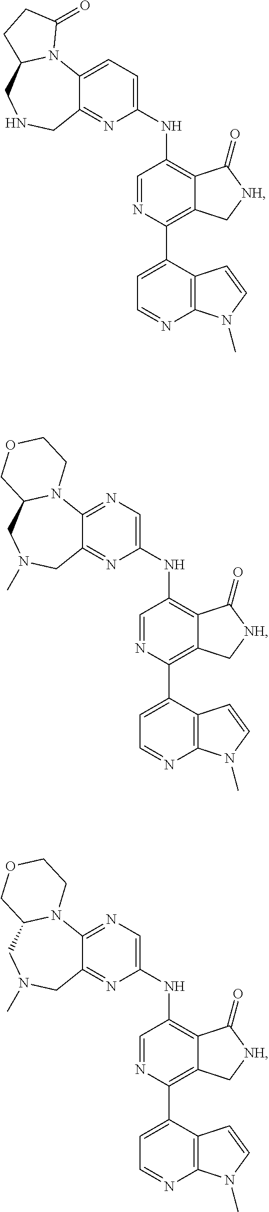 [SMILES: Cn1ccc2c(-c3ncc(Nc4ccc5c(n4)CNC[C@H]4CCC(=O)N54)c4c3CNC4=O)ccnc21 ; CN1Cc2nc(Nc3cnc(-c4ccnc5c4ccn5C)c4c3C(=O)NC4)cnc2N2CCOC[C@@H]2C1 ; CN1Cc2nc(Nc3cnc(-c4ccnc5c4ccn5C)c4c3C(=O)NC4)cnc2N2CCOC[C@H]2C1]

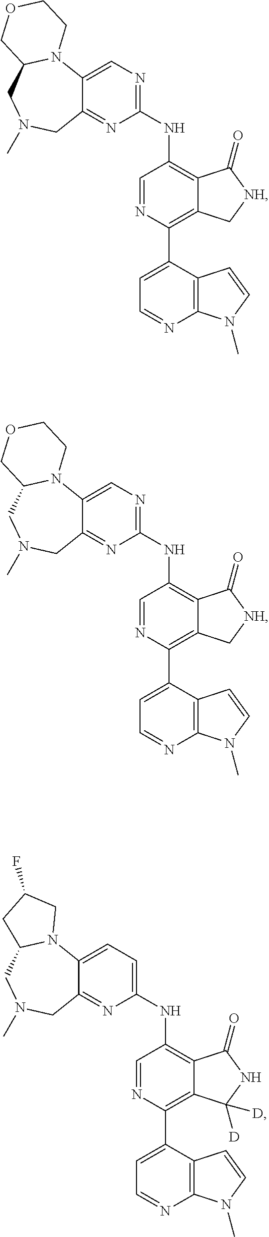

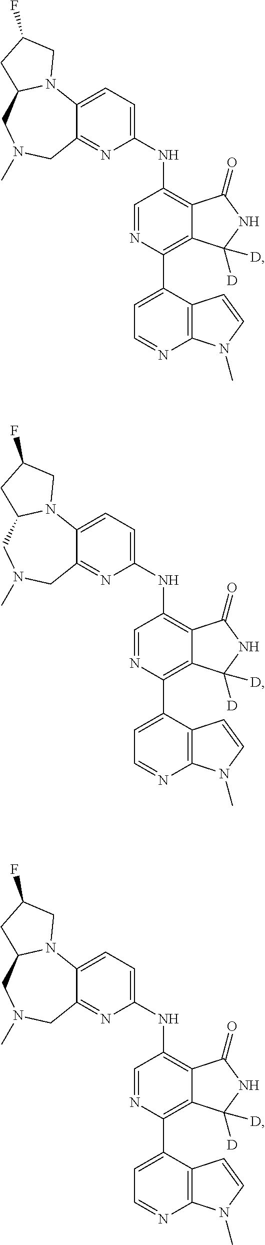

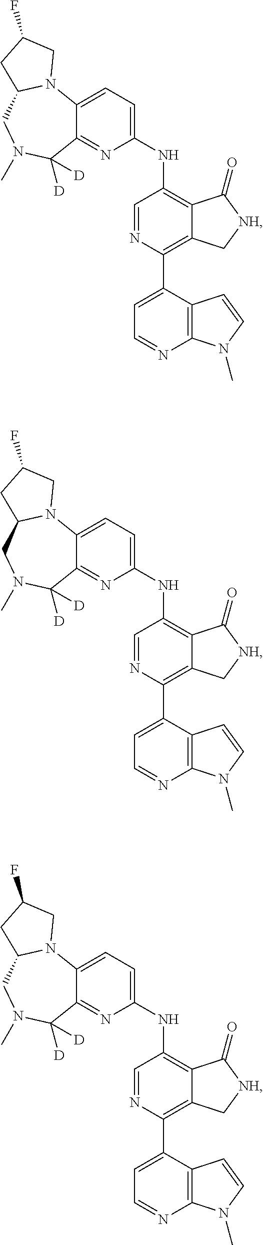 [SMILES: [2H]C1([2H])c2nc(Nc3cnc(-c4ccnc5c4ccn5C)c4c3C(=O)NC4)ccc2N2C[C@@H](F)C[C@H]2CN1C ; [2H]C1([2H])c2nc(Nc3cnc(-c4ccnc5c4ccn5C)c4c3C(=O)NC4)ccc2N2C[C@@H](F)C[C@@H]2CN1C ; [2H]C1([2H])c2nc(Nc3cnc(-c4ccnc5c4ccn5C)c4c3C(=O)NC4)ccc2N2C[C@H](F)C[C@H]2CN1C]

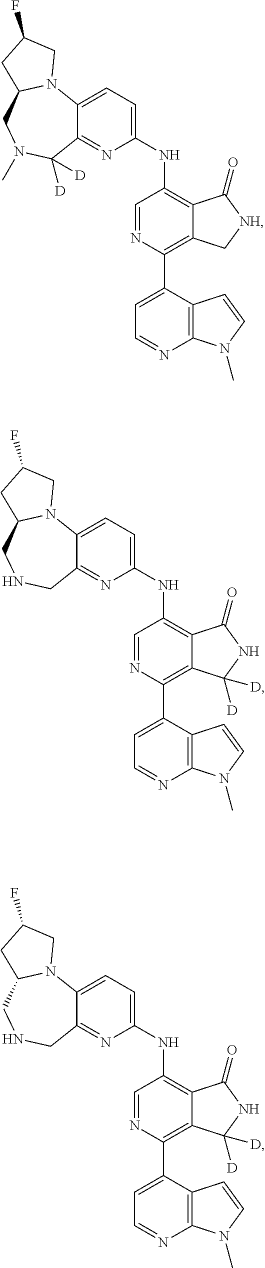

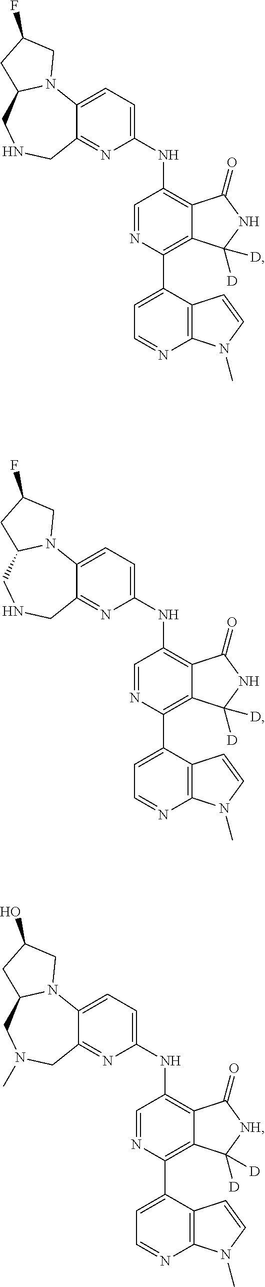

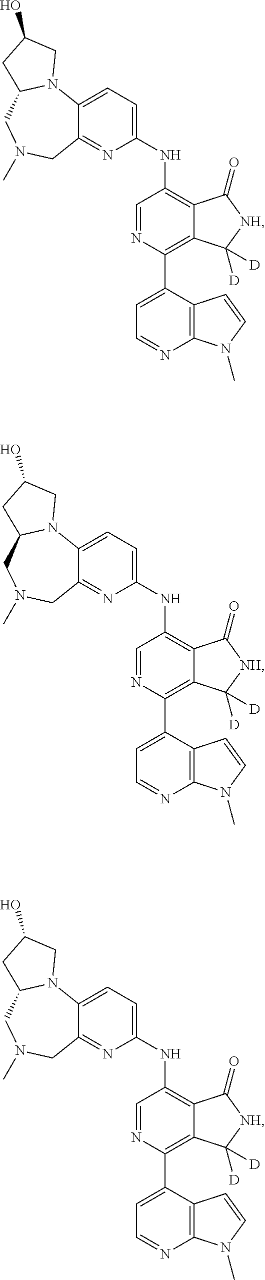 [SMILES: [2H]C1([2H])NC(=O)c2c(Nc3ccc4c(n3)CN(C)C[C@@H]3C[C@@H](O)CN43)cnc(-c3ccnc4c3ccn4C)c21 ; [2H]C1([2H])NC(=O)c2c(Nc3ccc4c(n3)CN(C)C[C@H]3C[C@H](O)CN43)cnc(-c3ccnc4c3ccn4C)c21 ; [2H]C1([2H])NC(=O)c2c(Nc3ccc4c(n3)CN(C)C[C@@H]3C[C@H](O)CN43)cnc(-c3ccnc4c3ccn4C)c21]

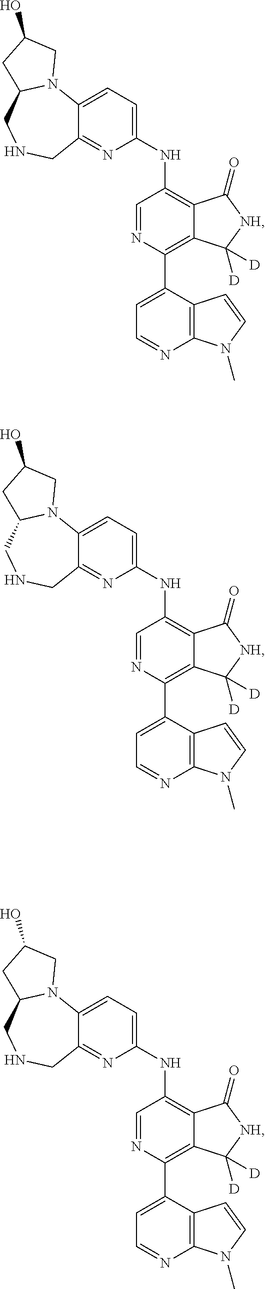 [SMILES: [2H]C1([2H])NC(=O)c2c(Nc3ccc4c(n3)CNC[C@H]3C[C@@H](O)CN43)cnc(-c3ccnc4c3ccn4C)c21 ; [2H]C1([2H])NC(=O)c2c(Nc3ccc4c(n3)CNC[C@@H]3C[C@@H](O)CN43)cnc(-c3ccnc4c3ccn4C)c21 ; [2H]C1([2H])NC(=O)c2c(Nc3ccc4c(n3)CNC[C@H]3C[C@H](O)CN43)cnc(-c3ccnc4c3ccn4C)c21]

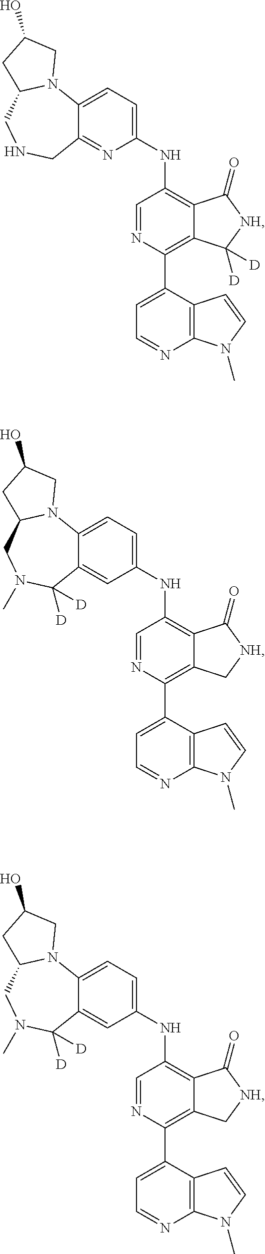 [SMILES: [2H]C1([2H])NC(=O)c2c(Nc3ccc4c(n3)CNC[C@@H]3C[C@H](O)CN43)cnc(-c3ccnc4c3ccn4C)c21 ; [2H]C1([2H])c2cc(Nc3cnc(-c4ccnc5c4ccn5C)c4c3C(=O)NC4)ccc2N2C[C@H](O)C[C@@H]2CN1C ; [2H]C1([2H])c2cc(Nc3cnc(-c4ccnc5c4ccn5C)c4c3C(=O)NC4)ccc2N2C[C@H](O)C[C@H]2CN1C]

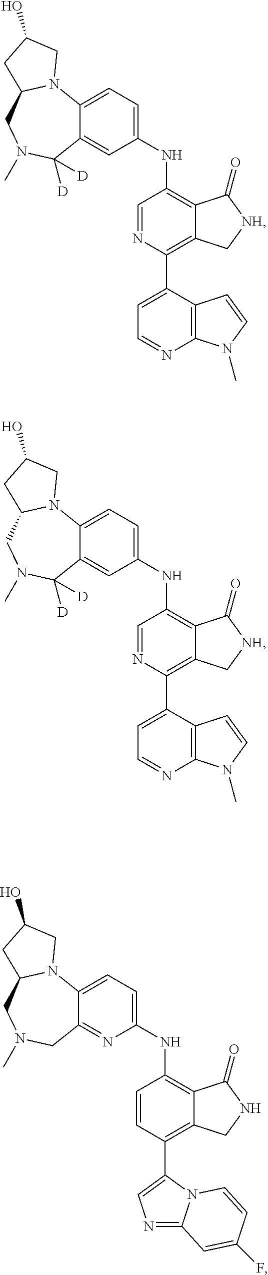 [SMILES: [2H]C1([2H])c2cc(Nc3cnc(-c4ccnc5c4ccn5C)c4c3C(=O)NC4)ccc2N2C[C@@H](O)C[C@@H]2CN1C ; [2H]C1([2H])c2cc(Nc3cnc(-c4ccnc5c4ccn5C)c4c3C(=O)NC4)ccc2N2C[C@@H](O)C[C@H]2CN1C ; CN1Cc2nc(Nc3ccc(-c4cnc5cc(F)ccn45)c4c3C(=O)NC4)ccc2N2C[C@H](O)C[C@@H]2C1]

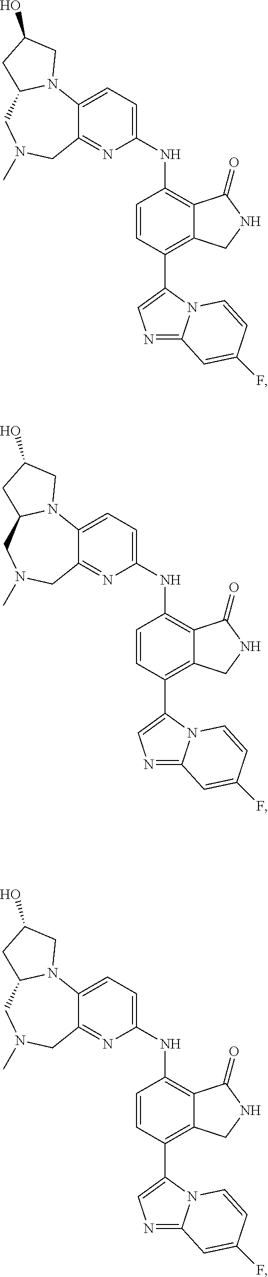 [SMILES: CN1Cc2nc(Nc3ccc(-c4cnc5cc(F)ccn45)c4c3C(=O)NC4)ccc2N2C[C@H](O)C[C@H]2C1 ; CN1Cc2nc(Nc3ccc(-c4cnc5cc(F)ccn45)c4c3C(=O)NC4)ccc2N2C[C@@H](O)C[C@@H]2C1 ; CN1Cc2nc(Nc3ccc(-c4cnc5cc(F)ccn45)c4c3C(=O)NC4)ccc2N2C[C@@H](O)C[C@H]2C1]

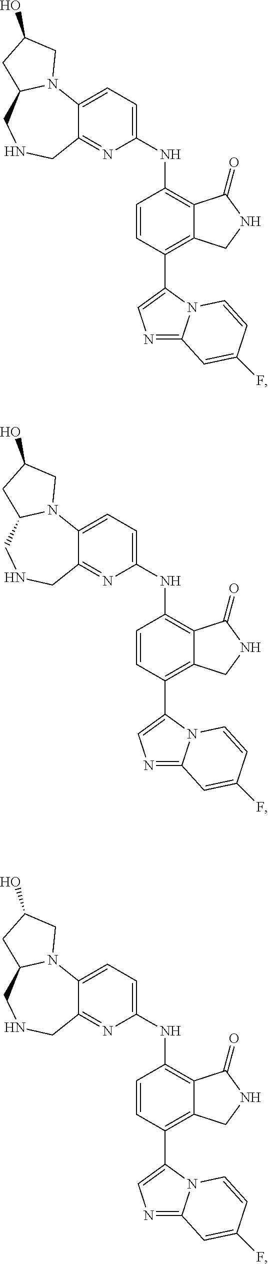 [SMILES: O=C1NCc2c(-c3cnc4cc(F)ccn34)ccc(Nc3ccc4c(n3)CNC[C@H]3C[C@@H](O)CN43)c21 ; O=C1NCc2c(-c3cnc4cc(F)ccn34)ccc(Nc3ccc4c(n3)CNC[C@@H]3C[C@@H](O)CN43)c21 ; O=C1NCc2c(-c3cnc4cc(F)ccn34)ccc(Nc3ccc4c(n3)CNC[C@H]3C[C@H](O)CN43)c21]

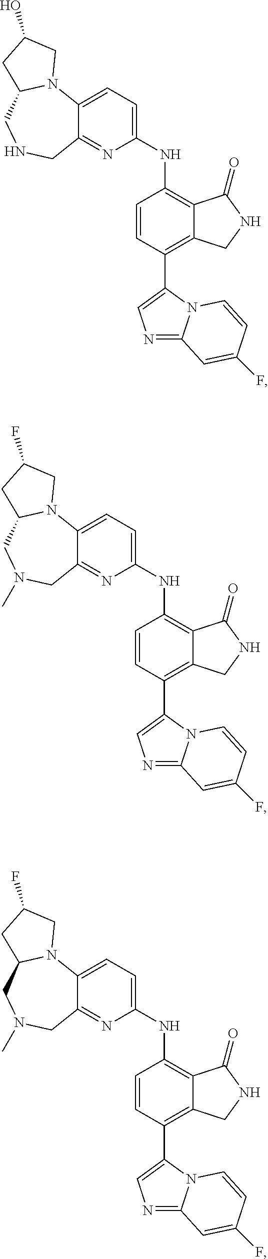 [SMILES: O=C1NCc2c(-c3cnc4cc(F)ccn34)ccc(Nc3ccc4c(n3)CNC[C@@H]3C[C@H](O)CN43)c21 ; CN1Cc2nc(Nc3ccc(-c4cnc5cc(F)ccn45)c4c3C(=O)NC4)ccc2N2C[C@@H](F)C[C@H]2C1 ; CN1Cc2nc(Nc3ccc(-c4cnc5cc(F)ccn45)c4c3C(=O)NC4)ccc2N2C[C@@H](F)C[C@@H]2C1]

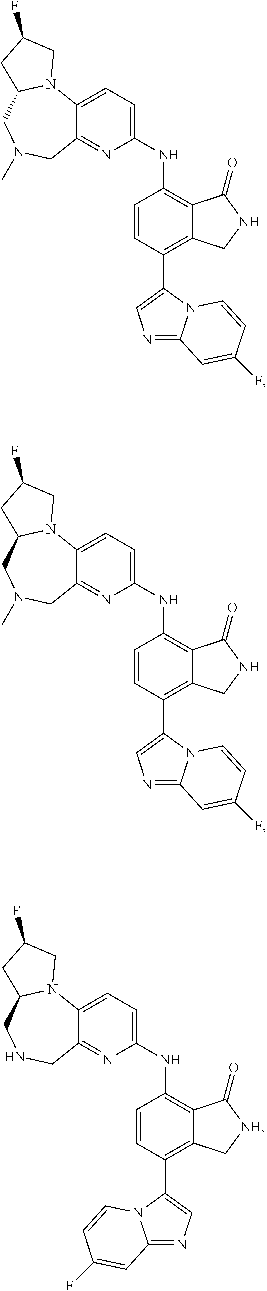 [SMILES: CN1Cc2nc(Nc3ccc(-c4cnc5cc(F)ccn45)c4c3C(=O)NC4)ccc2N2C[C@H](F)C[C@H]2C1 ; CN1Cc2nc(Nc3ccc(-c4cnc5cc(F)ccn45)c4c3C(=O)NC4)ccc2N2C[C@H](F)C[C@@H]2C1 ; O=C1NCc2c(-c3cnc4cc(F)ccn34)ccc(Nc3ccc4c(n3)CNC[C@H]3C[C@@H](F)CN43)c21]

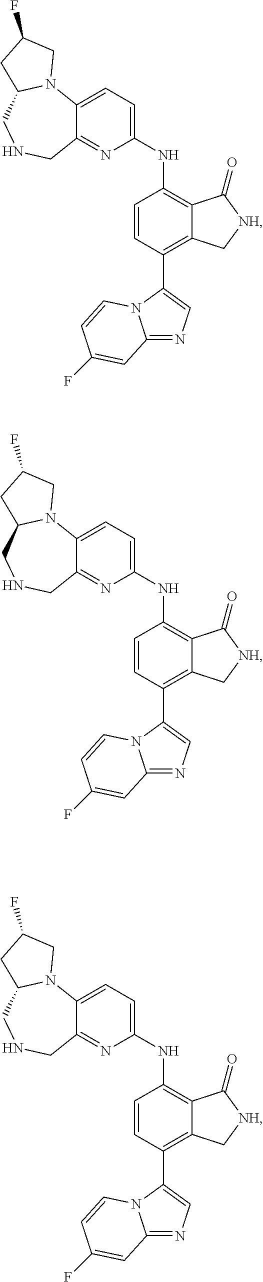 [SMILES: O=C1NCc2c(-c3cnc4cc(F)ccn34)ccc(Nc3ccc4c(n3)CNC[C@@H]3C[C@@H](F)CN43)c21 ; O=C1NCc2c(-c3cnc4cc(F)ccn34)ccc(Nc3ccc4c(n3)CNC[C@H]3C[C@H](F)CN43)c21 ; O=C1NCc2c(-c3cnc4cc(F)ccn34)ccc(Nc3ccc4c(n3)CNC[C@@H]3C[C@H](F)CN43)c21]

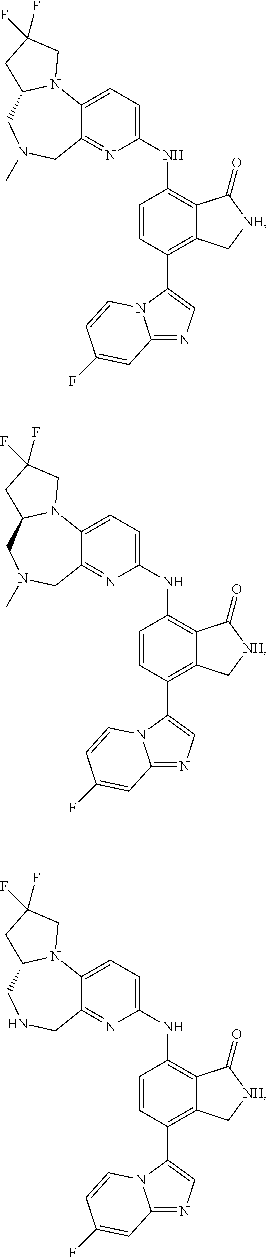 [SMILES: CN1Cc2nc(Nc3ccc(-c4cnc5cc(F)ccn45)c4c3C(=O)NC4)ccc2N2CC(F)(F)C[C@H]2C1 ; CN1Cc2nc(Nc3ccc(-c4cnc5cc(F)ccn45)c4c3C(=O)NC4)ccc2N2CC(F)(F)C[C@@H]2C1 ; O=C1NCc2c(-c3cnc4cc(F)ccn34)ccc(Nc3ccc4c(n3)CNC[C@@H]3CC(F)(F)CN43)c21]

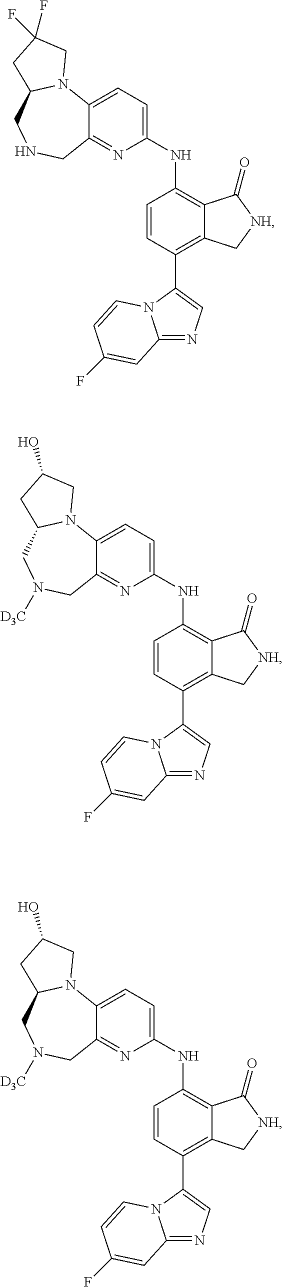 [SMILES: O=C1NCc2c(-c3cnc4cc(F)ccn34)ccc(Nc3ccc4c(n3)CNC[C@H]3CC(F)(F)CN43)c21 ; [2H]C([2H])([2H])N1Cc2nc(Nc3ccc(-c4cnc5cc(F)ccn45)c4c3C(=O)NC4)ccc2N2C[C@@H](O)C[C@H]2C1 ; [2H]C([2H])([2H])N1Cc2nc(Nc3ccc(-c4cnc5cc(F)ccn45)c4c3C(=O)NC4)ccc2N2C[C@@H](O)C[C@@H]2C1]

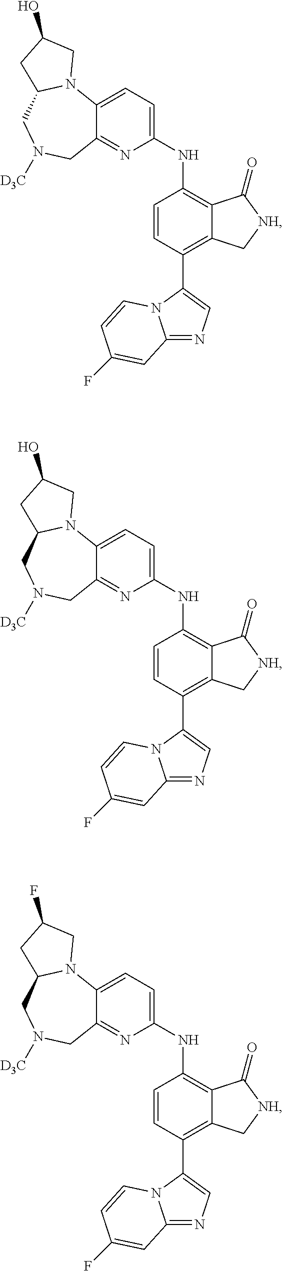 [SMILES: [2H]C([2H])([2H])N1Cc2nc(Nc3ccc(-c4cnc5cc(F)ccn45)c4c3C(=O)NC4)ccc2N2C[C@H](O)C[C@H]2C1 ; [2H]C([2H])([2H])N1Cc2nc(Nc3ccc(-c4cnc5cc(F)ccn45)c4c3C(=O)NC4)ccc2N2C[C@H](O)C[C@@H]2C1 ; [2H]C([2H])([2H])N1Cc2nc(Nc3ccc(-c4cnc5cc(F)ccn45)c4c3C(=O)NC4)ccc2N2C[C@H](F)C[C@@H]2C1]

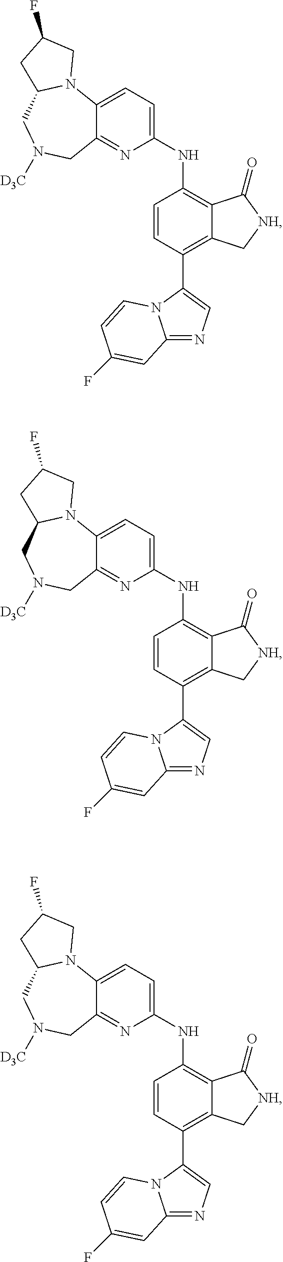 [SMILES: [2H]C([2H])([2H])N1Cc2nc(Nc3ccc(-c4cnc5cc(F)ccn45)c4c3C(=O)NC4)ccc2N2C[C@H](F)C[C@H]2C1 ; [2H]C([2H])([2H])N1Cc2nc(Nc3ccc(-c4cnc5cc(F)ccn45)c4c3C(=O)NC4)ccc2N2C[C@@H](F)C[C@@H]2C1 ; [2H]C([2H])([2H])N1Cc2nc(Nc3ccc(-c4cnc5cc(F)ccn45)c4c3C(=O)NC4)ccc2N2C[C@@H](F)C[C@H]2C1]

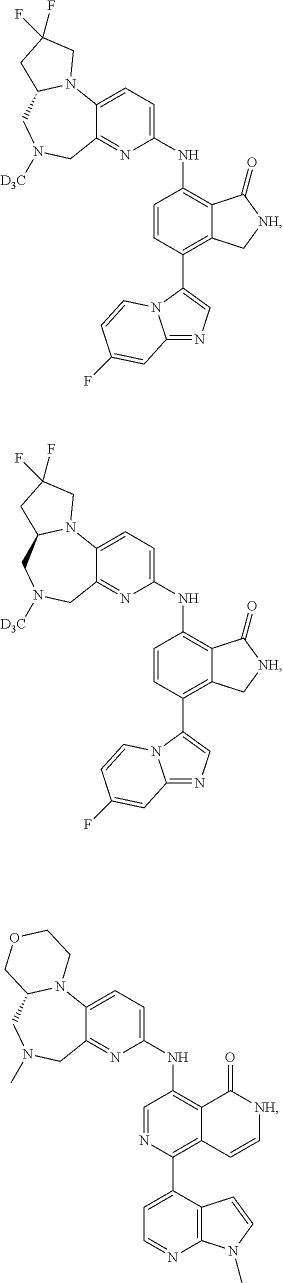 [SMILES: [2H]C([2H])([2H])N1Cc2nc(Nc3ccc(-c4cnc5cc(F)ccn45)c4c3C(=O)NC4)ccc2N2CC(F)(F)C[C@H]2C1 ; [2H]C([2H])([2H])N1Cc2nc(Nc3ccc(-c4cnc5cc(F)ccn45)c4c3C(=O)NC4)ccc2N2CC(F)(F)C[C@@H]2C1 ; CN1Cc2nc(Nc3cnc(-c4ccnc5c4ccn5C)c4cc[nH]c(=O)c34)ccc2N2CCOC[C@H]2C1]

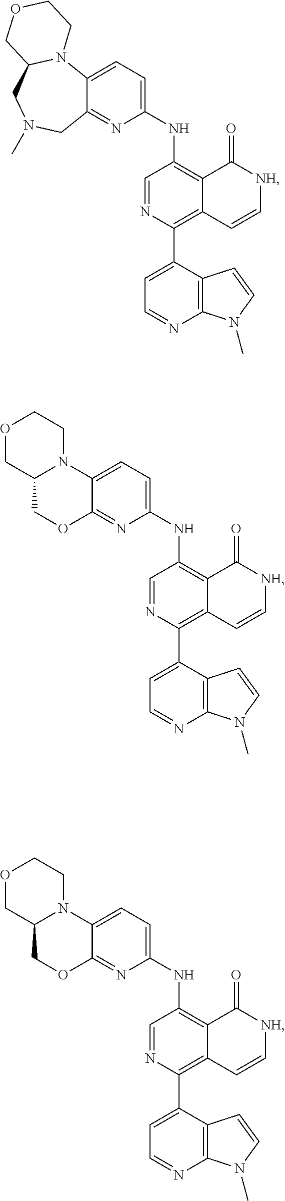 [SMILES: CN1Cc2nc(Nc3cnc(-c4ccnc5c4ccn5C)c4cc[nH]c(=O)c34)ccc2N2CCOC[C@@H]2C1 ; Cn1ccc2c(-c3ncc(Nc4ccc5c(n4)OC[C@@H]4COCCN54)c4c(=O)[nH]ccc34)ccnc21 ; Cn1ccc2c(-c3ncc(Nc4ccc5c(n4)OC[C@H]4COCCN54)c4c(=O)[nH]ccc34)ccnc21]

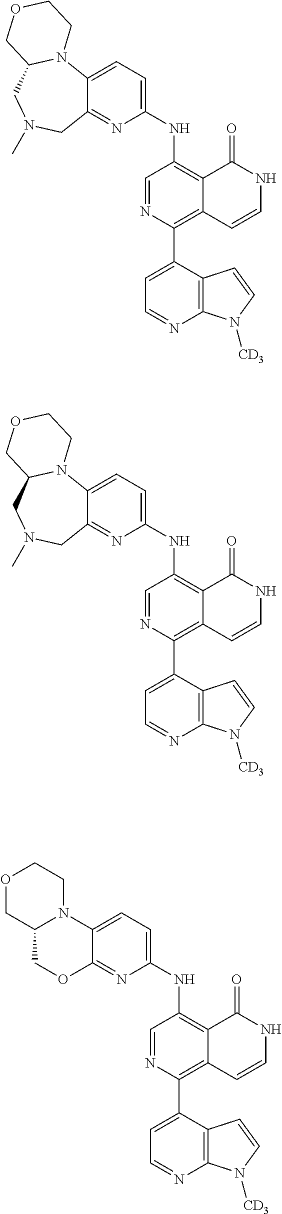 [SMILES: [2H]C([2H])([2H])n1ccc2c(-c3ncc(Nc4ccc5c(n4)CN(C)C[C@@H]4COCCN54)c4c(=O)[nH]ccc34)ccnc21 ; [2H]C([2H])([2H])n1ccc2c(-c3ncc(Nc4ccc5c(n4)CN(C)C[C@H]4COCCN54)c4c(=O)[nH]ccc34)ccnc21 ; [2H]C([2H])([2H])n1ccc2c(-c3ncc(Nc4ccc5c(n4)OC[C@@H]4COCCN54)c4c(=O)[nH]ccc34)ccnc21]

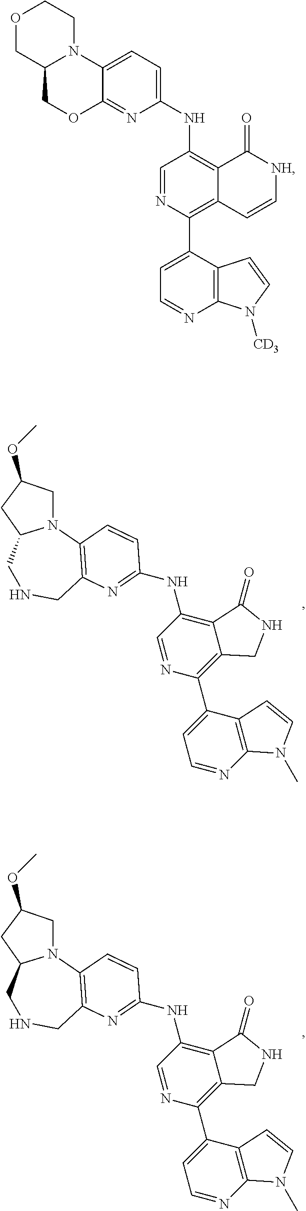 [SMILES: [2H]C([2H])([2H])n1ccc2c(-c3ncc(Nc4ccc5c(n4)OC[C@H]4COCCN54)c4c(=O)[nH]ccc34)ccnc21 ; CO[C@@H]1C[C@H]2CNCc3nc(Nc4cnc(-c5ccnc6c5ccn6C)c5c4C(=O)NC5)ccc3N2C1 ; CO[C@@H]1C[C@@H]2CNCc3nc(Nc4cnc(-c5ccnc6c5ccn6C)c5c4C(=O)NC5)ccc3N2C1]

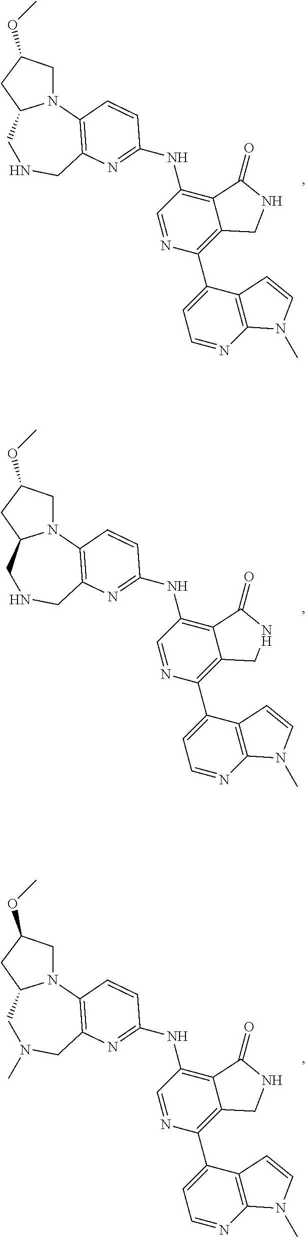 [SMILES: CO[C@H]1C[C@H]2CNCc3nc(Nc4cnc(-c5ccnc6c5ccn6C)c5c4C(=O)NC5)ccc3N2C1 ; CO[C@H]1C[C@@H]2CNCc3nc(Nc4cnc(-c5ccnc6c5ccn6C)c5c4C(=O)NC5)ccc3N2C1 ; CO[C@@H]1C[C@H]2CN(C)Cc3nc(Nc4cnc(-c5ccnc6c5ccn6C)c5c4C(=O)NC5)ccc3N2C1]

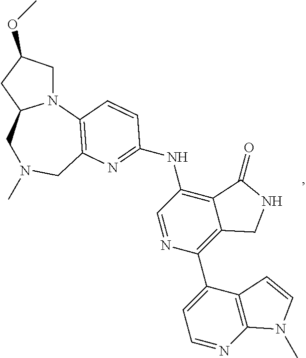 [SMILES: CO[C@@H]1C[C@@H]2CN(C)Cc3nc(Nc4cnc(-c5ccnc6c5ccn6C)c5c4C(=O)NC5)ccc3N2C1]

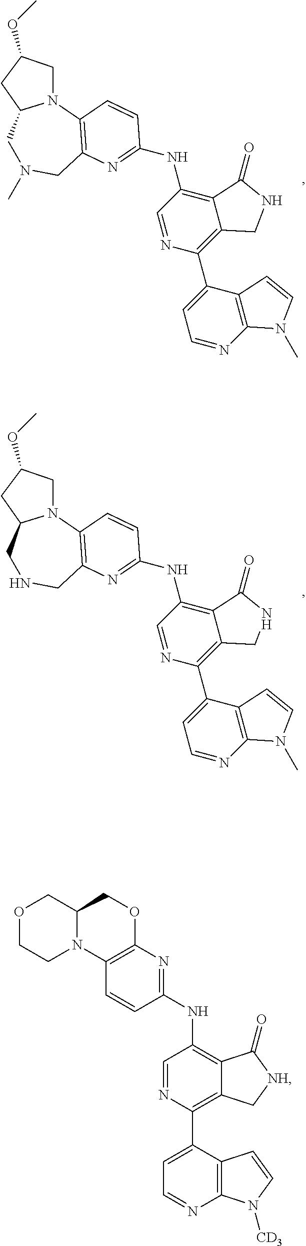 [SMILES: CO[C@H]1C[C@H]2CN(C)Cc3nc(Nc4cnc(-c5ccnc6c5ccn6C)c5c4C(=O)NC5)ccc3N2C1 ; CO[C@H]1C[C@@H]2CNCc3nc(Nc4cnc(-c5ccnc6c5ccn6C)c5c4C(=O)NC5)ccc3N2C1 ; [2H]C([2H])([2H])n1ccc2c(-c3ncc(Nc4ccc5c(n4)OC[C@@H]4COCCN54)c4c3CNC4=O)ccnc21]

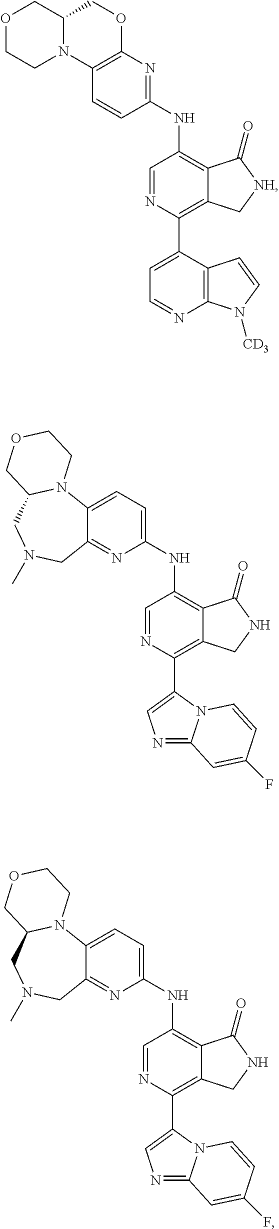 [SMILES: [2H]C([2H])([2H])n1ccc2c(-c3ncc(Nc4ccc5c(n4)OC[C@H]4COCCN54)c4c3CNC4=O)ccnc21 ; CN1Cc2nc(Nc3cnc(-c4cnc5cc(F)ccn45)c4c3C(=O)NC4)ccc2N2CCOC[C@H]2C1 ; CN1Cc2nc(Nc3cnc(-c4cnc5cc(F)ccn45)c4c3C(=O)NC4)ccc2N2CCOC[C@@H]2C1]

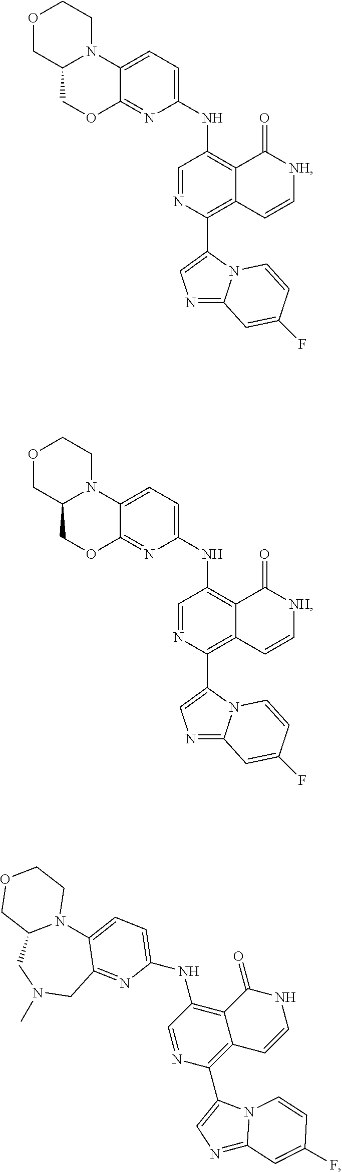 [SMILES: O=c1[nH]ccc2c(-c3cnc4cc(F)ccn34)ncc(Nc3ccc4c(n3)OC[C@@H]3COCCN43)c12 ; O=c1[nH]ccc2c(-c3cnc4cc(F)ccn34)ncc(Nc3ccc4c(n3)OC[C@H]3COCCN43)c12 ; CN1Cc2nc(Nc3cnc(-c4cnc5cc(F)ccn45)c4cc[nH]c(=O)c34)ccc2N2CCOC[C@H]2C1]

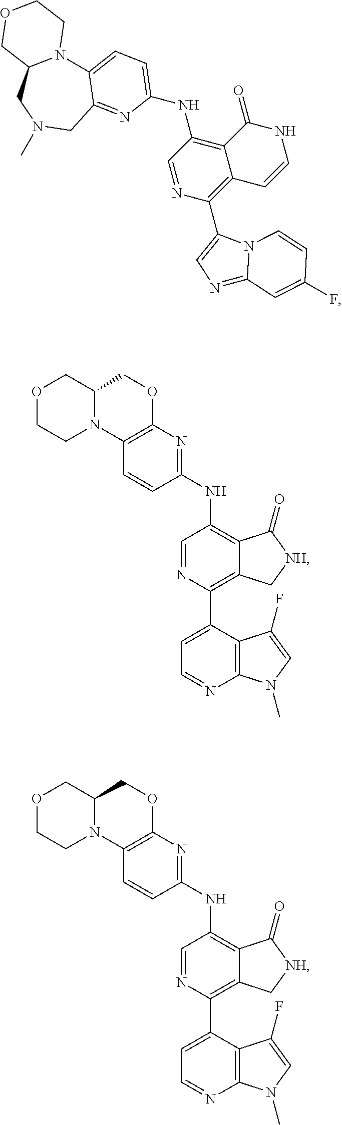 [SMILES: CN1Cc2nc(Nc3cnc(-c4cnc5cc(F)ccn45)c4cc[nH]c(=O)c34)ccc2N2CCOC[C@@H]2C1 ; Cn1cc(F)c2c(-c3ncc(Nc4ccc5c(n4)OC[C@H]4COCCN54)c4c3CNC4=O)ccnc21 ; Cn1cc(F)c2c(-c3ncc(Nc4ccc5c(n4)OC[C@@H]4COCCN54)c4c3CNC4=O)ccnc21]

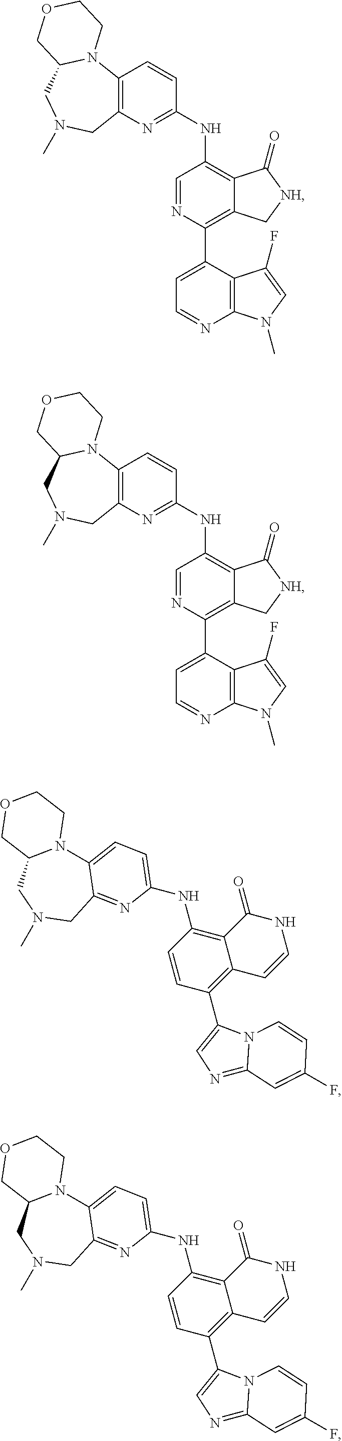 [SMILES: CN1Cc2nc(Nc3cnc(-c4ccnc5c4c(F)cn5C)c4c3C(=O)NC4)ccc2N2CCOC[C@H]2C1 ; CN1Cc2nc(Nc3cnc(-c4ccnc5c4c(F)cn5C)c4c3C(=O)NC4)ccc2N2CCOC[C@@H]2C1 ; CN1Cc2nc(Nc3ccc(-c4cnc5cc(F)ccn45)c4cc[nH]c(=O)c34)ccc2N2CCOC[C@H]2C1 ; CN1Cc2nc(Nc3ccc(-c4cnc5cc(F)ccn45)c4cc[nH]c(=O)c34)ccc2N2CCOC[C@@H]2C1]

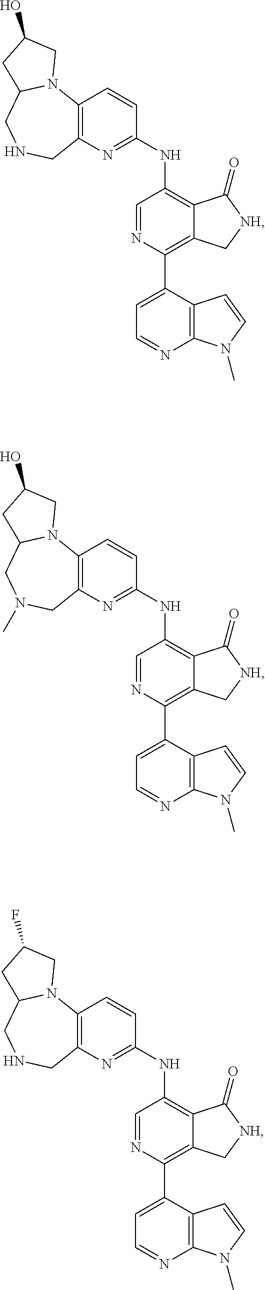 [SMILES: Cn1ccc2c(-c3ncc(Nc4ccc5c(n4)CNCC4C[C@@H](O)CN54)c4c3CNC4=O)ccnc21 ; CN1Cc2nc(Nc3cnc(-c4ccnc5c4ccn5C)c4c3C(=O)NC4)ccc2N2C[C@H](O)CC2C1 ; Cn1ccc2c(-c3ncc(Nc4ccc5c(n4)CNCC4C[C@H](F)CN54)c4c3CNC4=O)ccnc21]

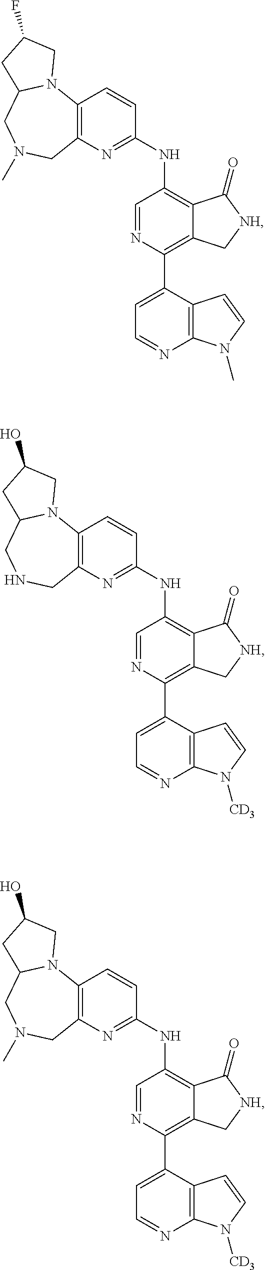 [SMILES: CN1Cc2nc(Nc3cnc(-c4ccnc5c4ccn5C)c4c3C(=O)NC4)ccc2N2C[C@@H](F)CC2C1 ; [2H]C([2H])([2H])n1ccc2c(-c3ncc(Nc4ccc5c(n4)CNCC4C[C@@H](O)CN54)c4c3CNC4=O)ccnc21 ; [2H]C([2H])([2H])n1ccc2c(-c3ncc(Nc4ccc5c(n4)CN(C)CC4C[C@@H](O)CN54)c4c3CNC4=O)ccnc21]

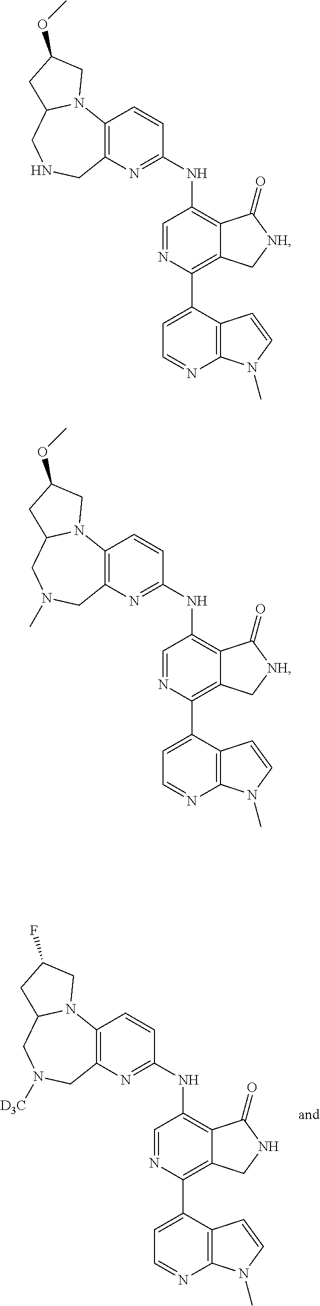 [SMILES: CO[C@@H]1CC2CNCc3nc(Nc4cnc(-c5ccnc6c5ccn6C)c5c4C(=O)NC5)ccc3N2C1 ; CO[C@@H]1CC2CN(C)Cc3nc(Nc4cnc(-c5ccnc6c5ccn6C)c5c4C(=O)NC5)ccc3N2C1 ; [2H]C([2H])([2H])N1Cc2nc(Nc3cnc(-c4ccnc5c4ccn5C)c4c3C(=O)NC4)ccc2N2C[C@@H](F)CC2C1]

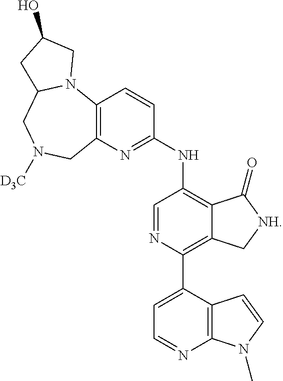 [SMILES: [2H]C([2H])([2H])N1Cc2nc(Nc3cnc(-c4ccnc5c4ccn5C)c4c3C(=O)NC4)ccc2N2C[C@H](O)CC2C1]

The present disclosure also provides use of the above compound, a stereoisomer or a pharmaceutically acceptable salt thereof in the manufacture of a medicament for the treatment of a solid tumor.

The present disclosure also provides use of the above compound, a stereoisomer or a pharmaceutically acceptable salt thereof in the manufacture of a medicament for the treatment of various types of tumors.

The present disclosure also provides use of the above compound, a stereoisomer or a pharmaceutically acceptable salt thereof in the manufacture of a medicament for the treatment of an HPK1-related disease.

The present disclosure also provides the following synthesis methods:

### Method 1:

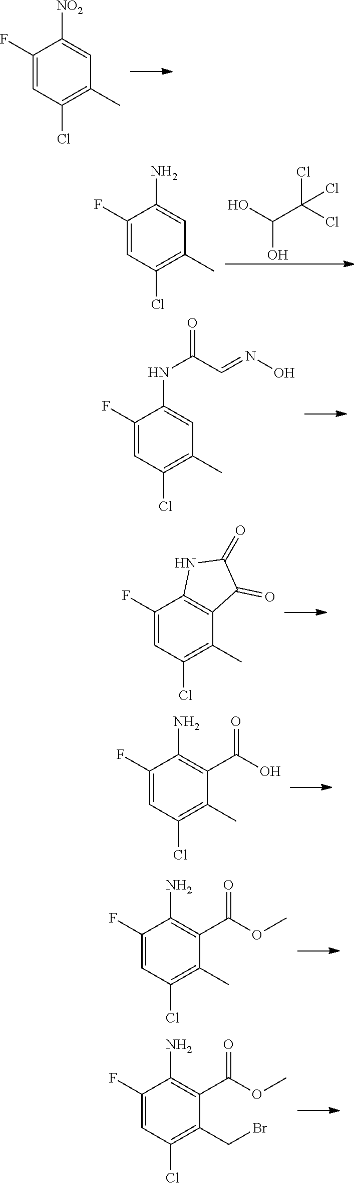

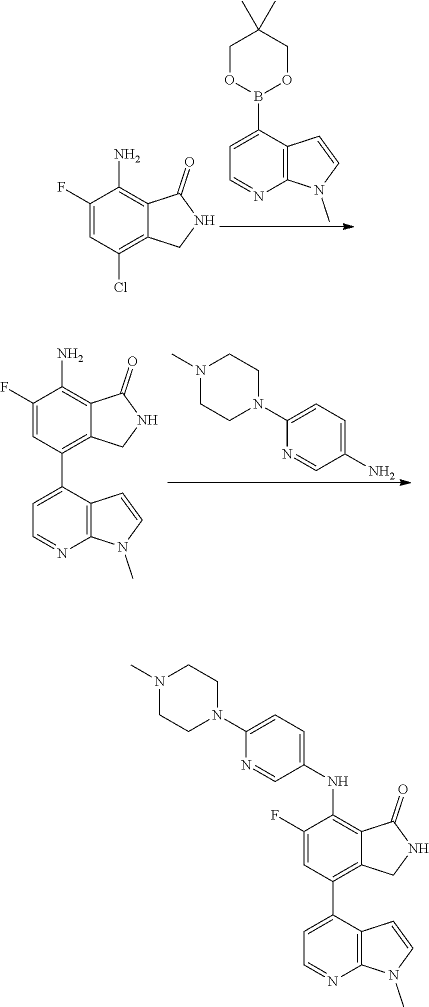

### Method 2:

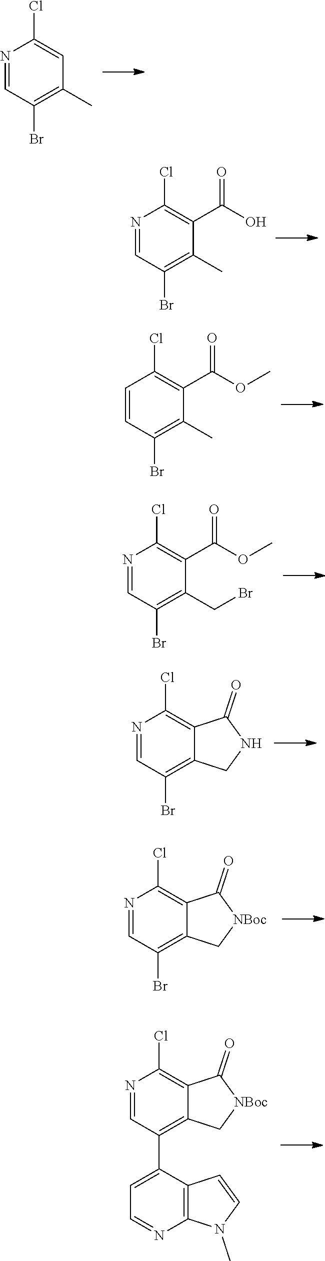

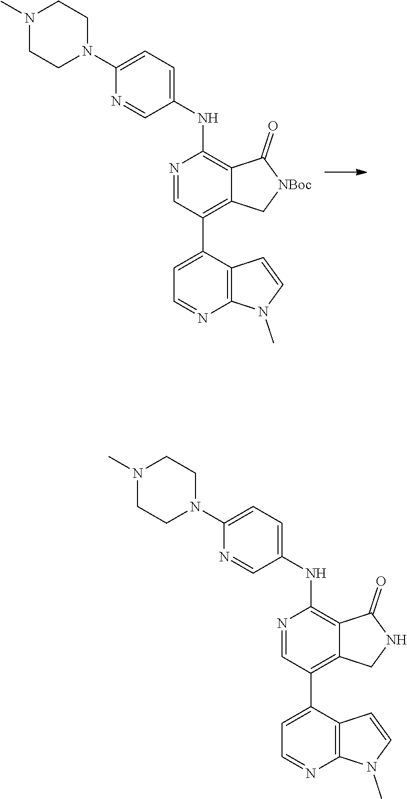 [SMILES: CN1CCN(c2ccc(Nc3ncc(-c4ccnc5c4ccn5C)c4c3C(=O)N(C(=O)OC(C)(C)C)C4)cn2)CC1 ; CN1CCN(c2ccc(Nc3ncc(-c4ccnc5c4ccn5C)c4c3C(=O)NC4)cn2)CC1]

### Method 3:

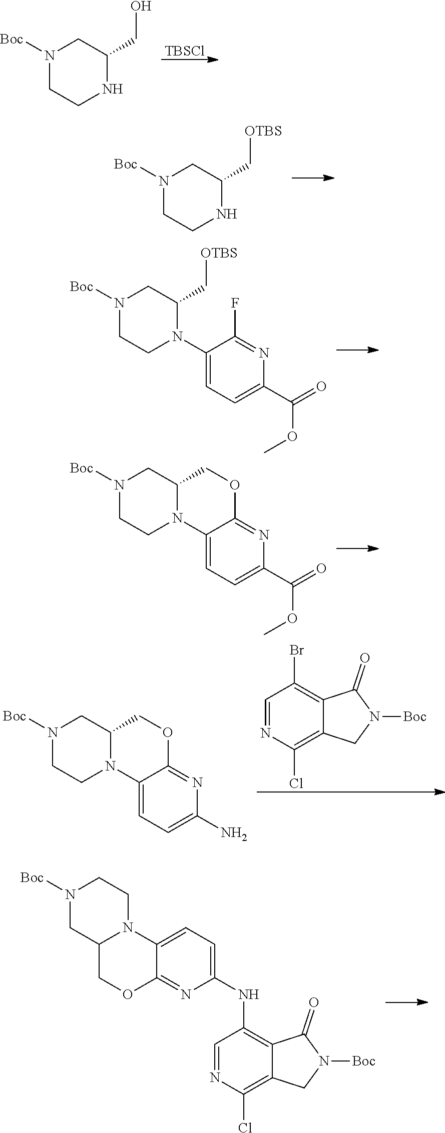

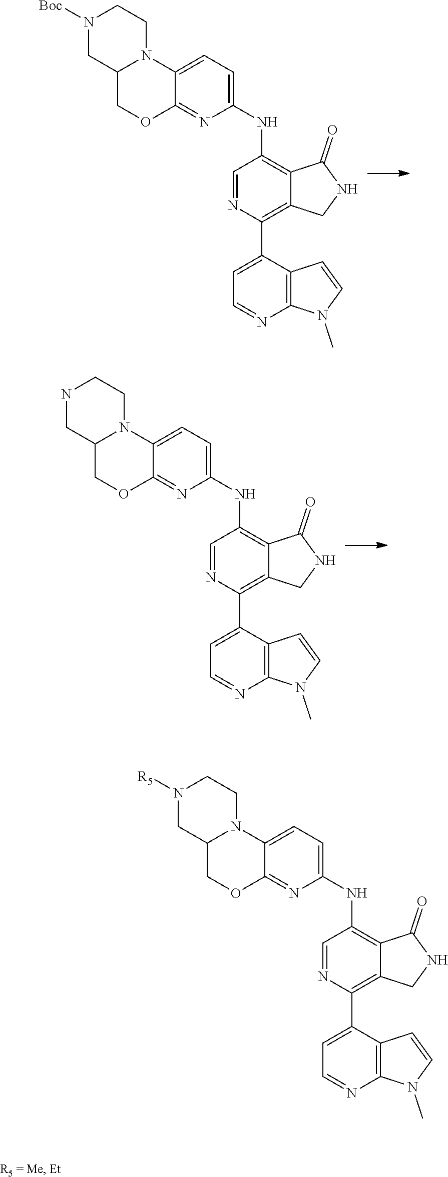

### Method 4:

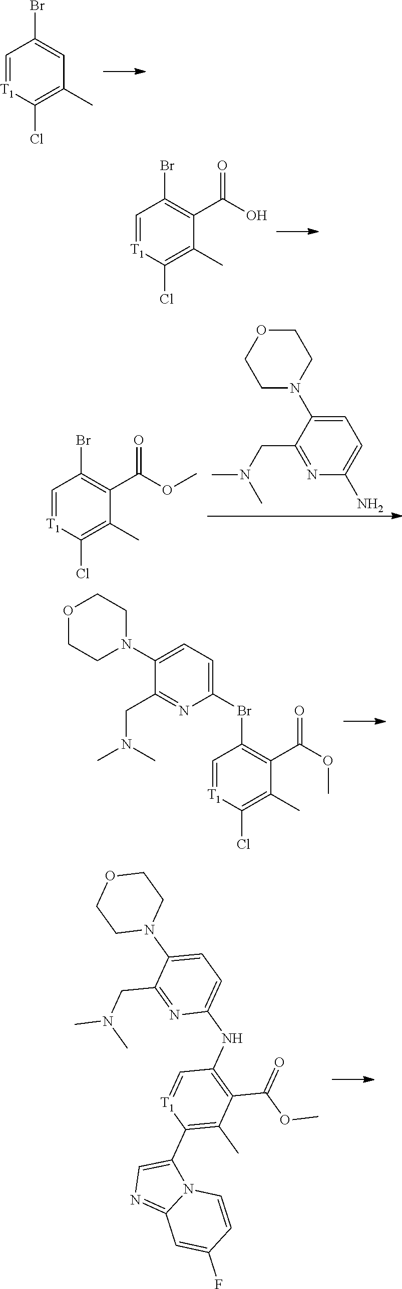

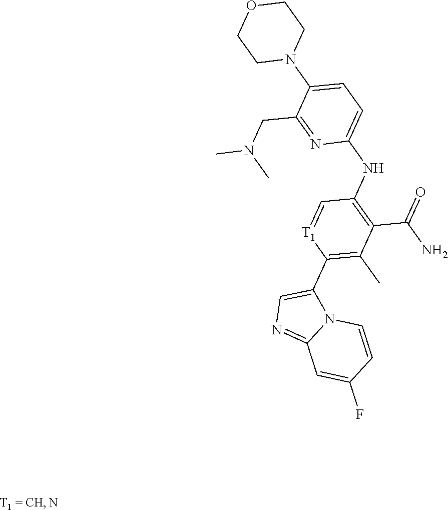

### Method 5:

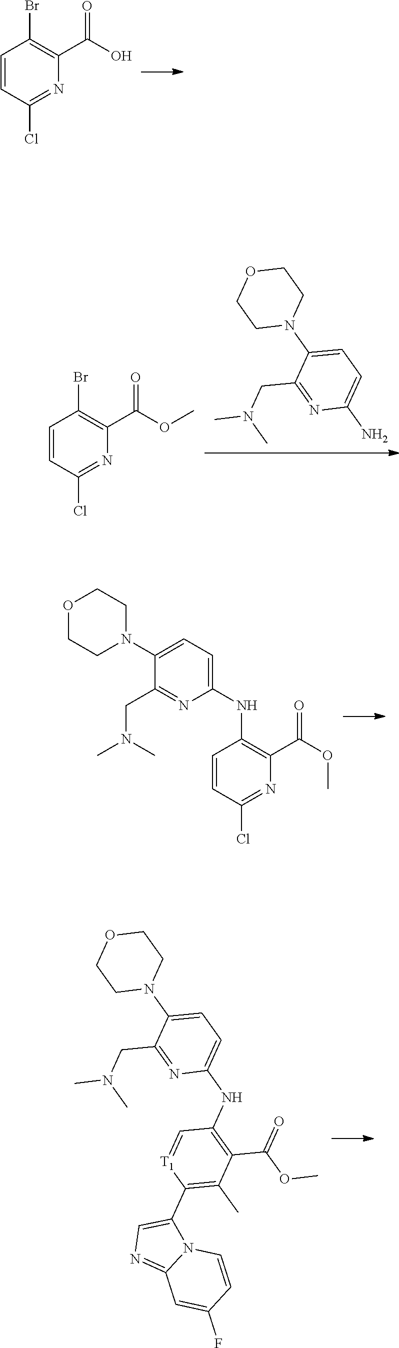

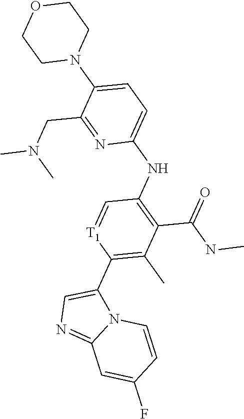

### Method 6:

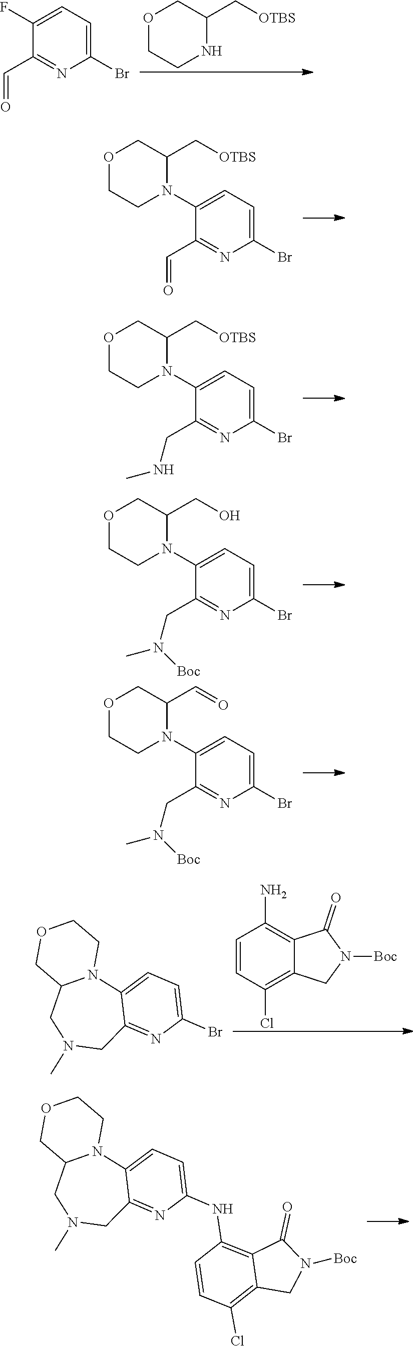

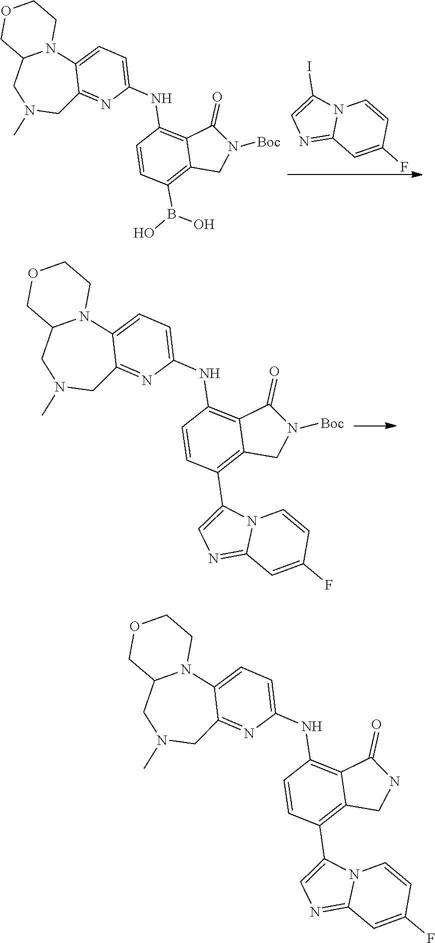

### Technical Effect

The compounds of the present disclosure have excellent HPK1 kinase inhibitory activity, weakly inhibit the subtype GLK (MAP4K3) of HPK1 kinase, and have excellent HPK1 selectivity; the compounds of the present disclosure have an effect of significantly increasing the IL-2 level in Jurkat cells, and have excellent inhibitory activity on the phosphorylation of SLP76 in Jurkat cells; the compounds of the present disclosure have good stability in human liver microsomes, and have good in vivo metabolic stability, excellent oral absorption drug exposure and good oral absorption bioavailability; the compounds of the present disclosure exhibit excellent membrane permeability in a study of cell membrane permeability.

### Definitions and Terms

Unless otherwise specified, the following terms and phrases used herein are intended to have the following meanings. A specific term or phrase should not be considered indefinite or unclear in the absence of a particular definition, but should be understood in the conventional sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, allergic reaction or other problems or complications, commensurate with a reasonable benefit/risk ratio.

The term “pharmaceutically acceptable salt” refers to a salt of the compound disclosed herein that is prepared by reacting the compound having a specific substituent disclosed herein with a relatively non-toxic acid or base. When the compound disclosed herein contains a relatively acidic functional group, a base addition salt can be obtained by bringing the compound into contact with a sufficient amount of base in a pure solution or a suitable inert solvent. When the compound disclosed herein contains a relatively basic functional group, an acid addition salt can be obtained by bringing the compound into contact with a sufficient amount of acid in a pure solution or a suitable inert solvent. Certain specific compounds disclosed herein contain both basic and acidic functional groups and can be converted to any base or acid addition salt.

The pharmaceutically acceptable salt disclosed herein can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

Unless otherwise specified, the term “isomer” is intended to include geometric isomers, cis-trans isomers, stereoisomers, enantiomers, optical isomers, diastereomers and tautomers.

The compound disclosed herein may be present in a specific geometric or stereoisomeric form. The present disclosure contemplates all such compounds, including cis and trans isomer, (−)- and (+)-enantiomer, (R)- and (S)-enantiomer, diastereomer, (D)-isomer, (L)-isomer, and racemic mixture and other mixtures, for example, an enantiomer or diastereomer enriched mixture, all of which are encompassed within the scope disclosed herein. The substituent such as alkyl may have an additional asymmetric carbon atom. All these isomers and mixtures thereof are encompassed within the scope disclosed herein.

Unless otherwise specified, the term “enantiomer” or “optical isomer” refers to stereoisomers that are in a mirrored relationship with each other.

Unless otherwise specified, the term “cis-trans isomer” or “geometric isomer” is produced by the inability of a double bond or a single bond between ring-forming carbon atoms to rotate freely.

Unless otherwise specified, the term “diastereomer” refers to a stereoisomer which has two or more chiral centers in a molecule and is in a non-mirrored relationship between molecules.

Unless otherwise specified, “(+)” means dextroisomer, “(−)” means levoisomer, and “(±)” means racemate.

Unless otherwise specified, a wedged solid bond ( [symbol] ) and a wedged dashed bond ( [symbol] ) indicate the absolute configuration of a stereocenter; a straight solid bond ( [symbol] ) and a straight dashed bond ( [symbol] ) indicate the relative configuration of a stereocenter; a wavy line ( [symbol] ) indicates a wedged solid bond ( [symbol] ) or a wedged dashed bond ( [symbol] ); or a wavy line ( [symbol] ) indicates a straight solid bond ( [symbol] ) or a straight dashed bond ( [symbol] ).

Unless otherwise specified, the term “tautomer” or “tautomeric form” refers to isomers of different functional groups that are in dynamic equilibrium at room temperature and can rapidly interconvert. If tautomerism is possible (e.g., in solution), a chemical equilibrium of tautomers can be reached. For example, proton tautomer (also known as prototropic tautomer) includes interconversion through proton migration, such as ketone-enol isomerization and imine-enamine isomerization. Valence tautomer includes interconversion through recombination of some bonding electrons. A specific example of ketone-enol tautomerization is interconversion of pentane-2,4-dione and 4-hydroxy-3-penten-2-one tautomeric isomers.

Unless otherwise specified, the term “enriched in one isomer”, “isomer enriched”, “enriched in one enantiomer” or “enantiomeric enriched” means that the content of one isomer or enantiomer is less than 100%, and the content of the isomer or enantiomer is 60% or more, or 70% or more, or 80% or more, or 90% or more, or 95% or more, or 96% or more, or 97% or more, or 98% or more, or 99% or more, or 99.5% or more, or 99.6% or more, or 99.7% or more, or 99.8% or more, or 99.9% or more.

Unless otherwise specified, the term “isomer excess” or “enantiomeric excess” refers to the difference between the relative percentages of two isomers or two enantiomers. For example, if one isomer or enantiomer is present in an amount of 90% and the other isomer or enantiomer is present in an amount of 10%, the isomer or enantiomeric excess (ee value) is 80%.

Optically active (R)- and (S)-isomer, or D and L isomer can be prepared using chiral synthesis or chiral reagents or other conventional techniques. If one kind of enantiomer of certain compound disclosed herein is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to give the pure enantiomer. In addition, the enantiomer and the diastereomer are generally isolated through chromatography which uses a chiral stationary phase and optionally combines with a chemical derivative method (for example, carbamate generated from amine).

The compounds disclosed herein may contain an unnatural proportion of atomic isotopes at one or more of the atoms that make up the compounds. For example, a compound may be labeled with a radioisotope such as tritium (³H), iodine-125 (¹²⁵I) or C-14 (¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs have advantages of reduced toxic side effects, increased drug stability, enhanced efficacy, and prolonged biological half-life of drugs. All changes in the isotopic composition of compounds disclosed herein, regardless of radioactivity, are included within the scope of the present disclosure.

The term “optional” or “optionally” means that the subsequently described event or circumstance may but need not occur, and that the description includes instances where said event or circumstance occurs and instances where said event or circumstance does not occur.

The term “substituted” means one or more than one hydrogen atom (s) on a specific atom are substituted by a substituent, including deuterium and hydrogen variants, as long as the valence of the specific atom is normal and the substituted compound is stable. When the substituent is oxo (i.e., ═O), it means two hydrogen atoms are substituted. Positions on an aromatic ring cannot be substituted by oxo.

The term “optionally substituted” means an atom can be substituted by a substituent or not, unless otherwise specified, the species and number of the substituent may be arbitrary so long as being chemically achievable.

When any variable (such as R) occurs in the constitution or structure of the compound more than once, the definition of the variable at each occurrence is independent. Thus, for example, if a group is substituted by 0-2 R, the group can be optionally substituted by up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When the number of a substituent is 0, it means that the substituent does not exist. For example, -A-(R)₀ means that the structure is actually -A.

When a substituent is vacant, it means that the substituent does not exist. For example, when X in A-X is vacant, it means that the structure is actually A.

When one of the variables is a single bond, it means that the two groups linked by the single bond are connected directly. For example, when L in A-L-Z represents a single bond, the structure of A-L-Z is actually A-Z.

When the bond of a substituent can be cross-linked to two or more atoms on a ring, such a substituent can be bonded to any atom on the ring, for example, the structural unit

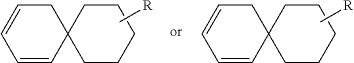

means that the substitution with substituent R can occur at any position on cyclohexyl or cyclohexadiene. When an enumerative substituent does not indicate through which atom it is linked to the substituted group, such substituent can be bonded through any of its atoms. For example, a pyridyl group as a substituent may be linked to the substituted group through any one of carbon atoms on the pyridine ring.

When an enumerative linking group does not indicate its linking direction, its linking direction is arbitrary. For example, when the linking group L in

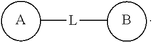

is -M-W—, the -M-W— can be linked to the ring A and the ring B in the same direction as the reading order from left to right to constitute

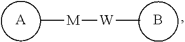

or can be linked to the ring A and the ring B in the reverse direction as the reading order from left to right to constitute

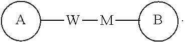

A combination of the linking groups, substituents and/or variants thereof is allowed only when such combination can result in a stable compound.

Unless otherwise specified, when a group has one or more connectable sites, any one or more sites of the group can be connected to other groups through chemical bonds. Where the connection position of the chemical bond is variable, and there is H atom(s) at a connectable site(s), when the connectable site(s) having H atom(s) is connected to the chemical bond, the number of H atom(s) at this site will correspondingly decrease as the number of the connected chemical bond increases, and the group will become a group of corresponding valence. The chemical bond between the site and other groups can be represented by a straight solid bond ( [symbol] ), a straight dashed bond ( [symbol] ), or a wavy line ( [symbol] ). For example, the straight solid bond in —OCH₃ indicates that the group is connected to other groups through the oxygen atom in the group; the straight dashed bond in

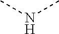

indicates that the group is connected to other groups through two ends of the nitrogen atom in the group; the wavy line in

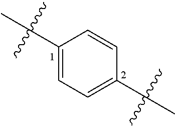 [SMILES: *c1ccc(*)cc1]

indicates that the group is connected to other groups through the 1- and 2-carbon atoms in the phenyl group;

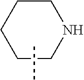

indicates that any connectable site on the piperidyl group can be connected to other groups through one chemical bond, including at least four connection ways,

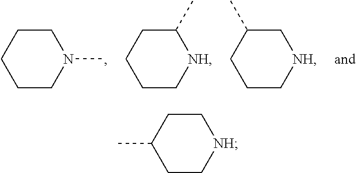

even if a H atom is drawn on —N—,

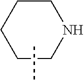

still includes the connection way of

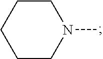

it's just that when one chemical bond is connected, the H at this site will be reduced by one, and the group will become the corresponding monovalent piperidyl group.

Unless otherwise specified, the number of atoms in a ring is generally defined as the number of ring members, e.g., “5-7 membered ring” refers to a “ring” having 5-7 ring atoms arranged.

Unless otherwise specified, the D in the present disclosure represents deuterium (²H).

Unless otherwise specified, the term “halo” or “halogen” by itself or as a part of another substituent, means a fluorine, chlorine, bromine, or iodine atom.

Unless otherwise specified, the term “C₁₋₃ alkyl” is used to indicate a linear or branched saturated hydrocarbon group consisting of 1 to 3 carbon atoms. The C₁₋₃ alkyl group includes C₁-2 and C₂₋₃ alkyl groups and the like. It may be monovalent (e.g., methyl), divalent (e.g., methylene) or multivalent (e.g., methenyl). Examples of C₁₋₃ alkyl groups include, but are not limited to, methyl (Me), ethyl (Et), propyl (including n-propyl and isopropyl), and the like.

Unless otherwise specified, the term “C₁₋₃ alkoxy” means alkyl groups containing 1 to 3 carbon atoms and attached to the remainder of a molecule by an oxygen atom. The C₁₋₃ alkoxy group includes C₁₋₂, C₂₋₃, C₃, and C₂ alkoxy groups, and the like. Examples of C₁₋₃ alkoxy groups include, but are not limited to, methoxy, ethoxy, propoxy (including n-propoxy and isopropoxy), and the like.

Unless otherwise specified, the term “C₁₋₃ alkylamino” means alkyl groups containing 1 to 3 carbon atoms and attached to the remainder of a molecule by a nitrogen atom. The C₁₋₃ alkylamino group includes C₁₋₂, C₃, and C₂ alkylamino groups, and the like. Examples of C₁₋₃ alkylamino groups include, but are not limited to, —NHCH₃, —N(CH₃)₂, —NHCH₂CH₃, —N(CH₃)CH₂CH₃, —NHCH₂CH₂CH₃, —NHCH₂(CH₃)₂, and the like.

Unless otherwise specified, the term “heteroaryl having 5-membered ring fused to 6-membered ring” means a bicyclic group having a conjugated 71 electron system and consisting of a ring of 5 ring atoms fused to a ring of 6 ring atoms, wherein the ring of 5 ring atoms and the ring of 6 ring atoms share two ring atoms. 1, 2, 3 or 4 of the ring atoms are heteroatoms independently selected from O, S and N, and the rest are carbon atoms. Among the heteroatoms, the nitrogen atom is optionally quaternized, and the nitrogen and sulfur heteroatoms may be optionally oxidized (i.e., NO and S(O)ₚ, p is 1 or 2). The heteroaryl having 5-membered ring fused to 6-membered ring can be attached to the remainder of a molecule via a heteroatom or a carbon atom. Examples of the heteroaryl having 5-membered ring fused to 6-membered ring include, but are not limited to, pyrrolopyridinyl and imidazopyridinyl.

The structures of the compounds of the present disclosure can be confirmed by conventional methods well known to those skilled in the art. If the present disclosure relates to an absolute configuration of a compound, the absolute configuration can be confirmed by conventional techniques in the art, such as single crystal X-Ray diffraction (SXRD). In the single crystal X-Ray diffraction (SXRD), the diffraction intensity data of the cultivated single crystal is collected using a Bruker D8 venture diffractometer with a light source of CuKα radiation in a scanning mode of φ/ω scan; after collecting the relevant data, the crystal structure is further analyzed by the direct method (Shelxs97) to confirm the absolute configuration.

Compounds disclosed herein can be prepared by a variety of synthetic methods well known to those skilled in the art, including the following enumerative embodiment, the embodiment formed by the following enumerative embodiment in combination with other chemical synthesis methods, and the equivalent well known to those skilled in the art. Alternative embodiments include, but are not limited to examples disclosed herein.

Solvents used in the present disclosure are commercially available.

The present disclosure employs the following abbreviations: DCM represents dichloromethane, EA represents ethyl acetate, PE represents petroleum ether, THF represents tetrahydrofuran, DMF represents N,N-dimethylformamide, MeOH represents methanol, AcOH represents acetic acid, MTBE represents methyl tert-butyl ether, TFA represents trifluoroacetic acid, TEA represents triethylamine, DIEA represents N,N-diisopropylethylamine, Pd(OAc)₂ represents palladium(II) acetate, Pd(PPh₃)₄ represents tetrakis(triphenylphosphine)palladium, Pd(dppf)Cl₂·CH₂Cl₂ represents [1,1′-Bis(diphenylphosphino)ferrocene]dichloropalladium(II), complex with dichloromethane, Pd(dppf)Cl₂ represents [1,1′-Bis(diphenylphosphino)ferrocene]dichloropalladium(II), Pd₂(dba)₃ represents tris(dibenzylideneacetone)dipalladium, BINAP represents 2,2′-bis(diphenylphosphino)-1,1′-binaphthyl, XPhos Pd G2 represents chloro(2-dicyclohexylphosphino-2′,4′,6′-triisopropyl-1,1′-biphenyl)[2-(2′-amino-1,1′-biphenyl)]palladium(II), RuPhos Pd G2 represents chloro(2-dicyclohexylphosphino-2′,6′-diisopropoxy-1,1′-biphenyl) [2-(2′-amino-1,1′-biphenyl)]palladium(II), Xantphos Pd G4 represents methanesulfonato[4,5-bis(diphenylphosphino)-9,9-dimethylxanthene](2′-methylamino-1,1′-biphenyl-2-yl)palladium(II), Ruphos represents 2-dicyclohexylphosphino-2′,6′-diisopropoxy-1,1′-biphenyl, Xphos represents 2-dicyclohexylphosphino-2′,4′,6′-triisopropylbiphenyl, Xantphos represents 4,5-bis(diphenylphosphino)-9,9-dimethylxanthene, Dess-Martin periodinane represents 1,1,1-triacetoxy-1,1-dihydro-1,2-benziodoxol-3(1H)-one, Boc₂O represents di-tert-butyl dicarbonate, BocNH₂ represents tert-butyl carbamate, s-BuLi represents sec-butyllithium, NaBH(OAc)₃ represents sodium triacetoxyborohydride, HOBt represents 1-hydroxybenzotriazole, HATU represents O-(7-azabenzotriazol-1-yl)-N,N,N′,N′-tetramethyluronium hexafluorophosphate, Cs₂CO₃ represents cesium carbonate, BH₃·THF represents borane tetrahydrofuran solution, TfOH represents trifluoromethanesulfonic acid, ZnBr₂ represents zinc bromide, Raney-Ni represents Raney nickel, DAST represents diethylaminosulfur trifluoride, NaH represents sodium hydride, CD₃I represents deuterated iodomethane, KOAc represents potassium acetate, Mel represents iodomethane, TBSCl represents tert-butyldimethylsilyl chloride, (PMB)₂NH represents bis(p-methoxybenzyl)amine, Boc represents tert-butyloxycarbonyl, PMB represents p-methoxybenzyl, DMB represents 2,4-dimethoxybenzyl, Select-F represents 1-chloromethyl-4-fluoro-1,4-diazoniabicyclo[2.2.2]octane bis(tetrafluoroborate), and prep-HPLC represents preparative high performance liquid chromatography.

Compounds are named according to general naming principles in the art or by ChemDraw® software, and commercially available compounds are named with their vendor directory names.

### BRIEF DESCRIPTION OF DRAWINGS

FIG. 1 is the prediction of the binding mode of compound A to HPK1;

FIG. 2 is the prediction of the binding mode of compound B to HPK1;

FIG. 3 is the prediction of the binding mode of compound C to HPK1;

FIG. 4 is the prediction of the binding mode of compound D to HPK1;

FIG. 5 is the prediction of the binding mode of compound E to HPK1;

FIG. 6 is the prediction of the binding mode of compound F to HPK1;

FIG. 7 is the prediction of the binding mode of compound G to HPK1;

FIG. 8 is the prediction of the binding mode of compound H to HPK1;

FIG. 9 is the prediction of the binding mode of compound I to HPK1;

FIG. 10 is the prediction of the binding mode of compound J to HPK1;

FIG. 11 is the prediction of the binding mode of compound K to HPK1;

FIG. 12 is the prediction of the binding mode of compound L to HPK1;

FIG. 13 is the prediction of the binding mode of compound M to HPK1.

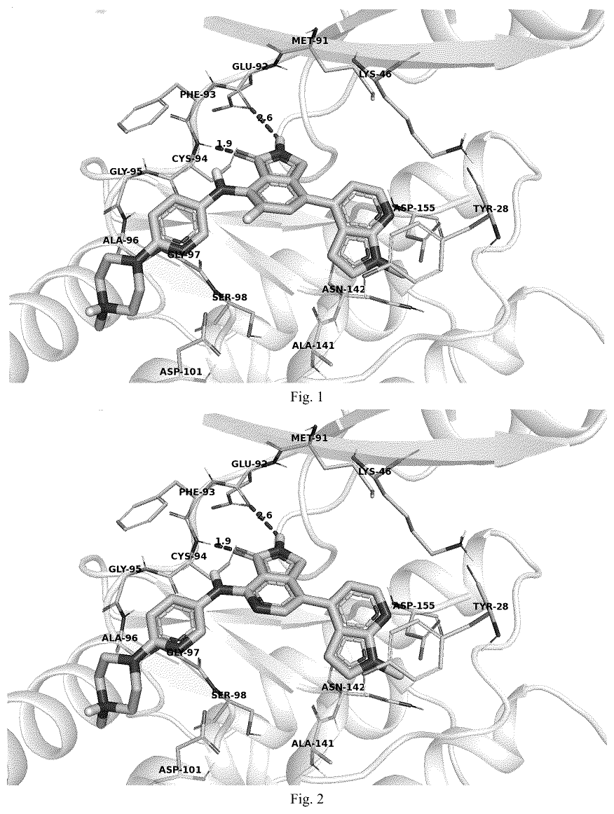

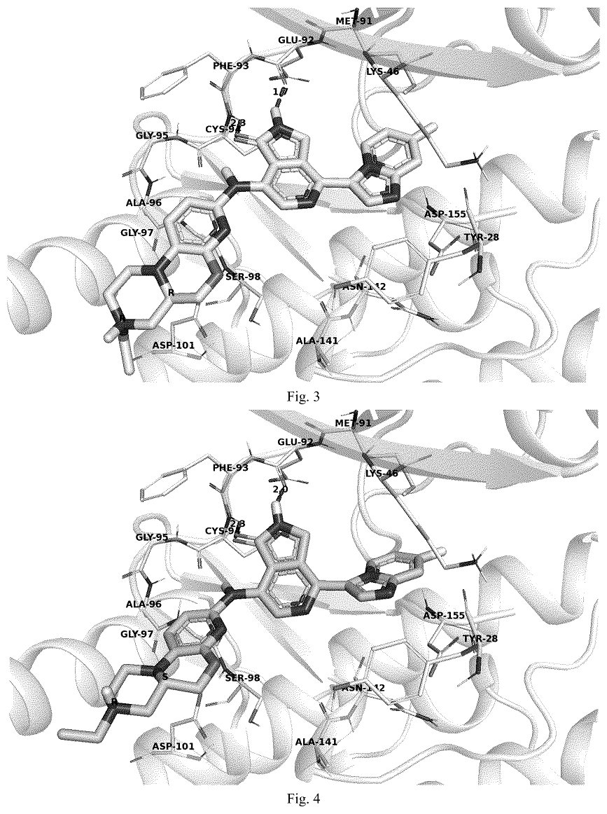

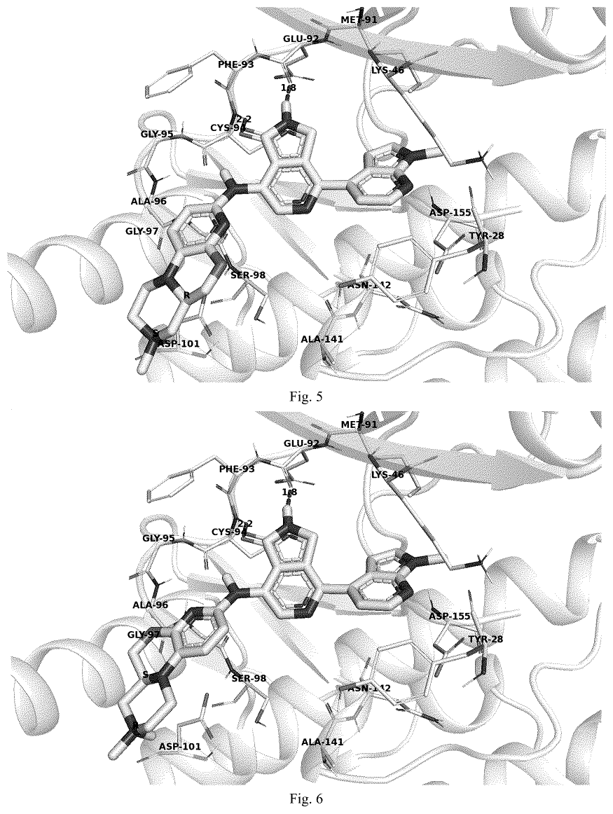

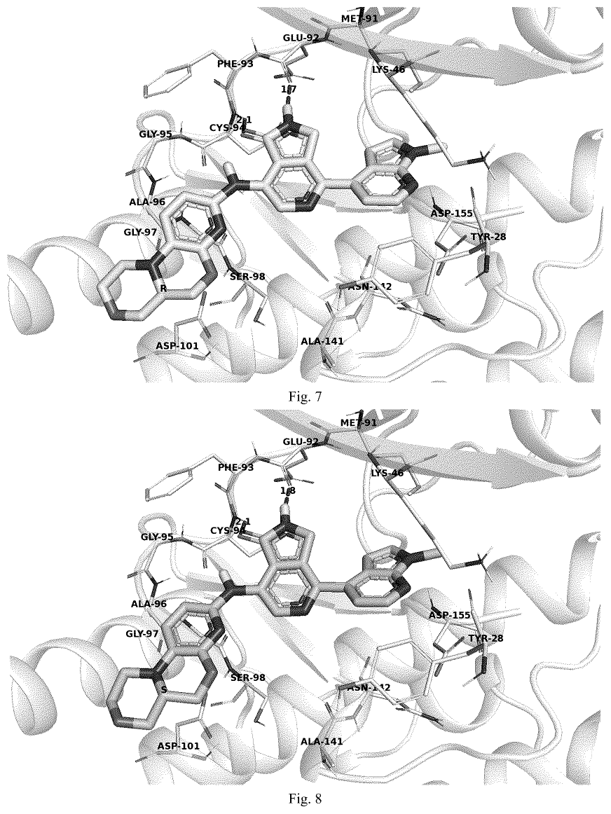

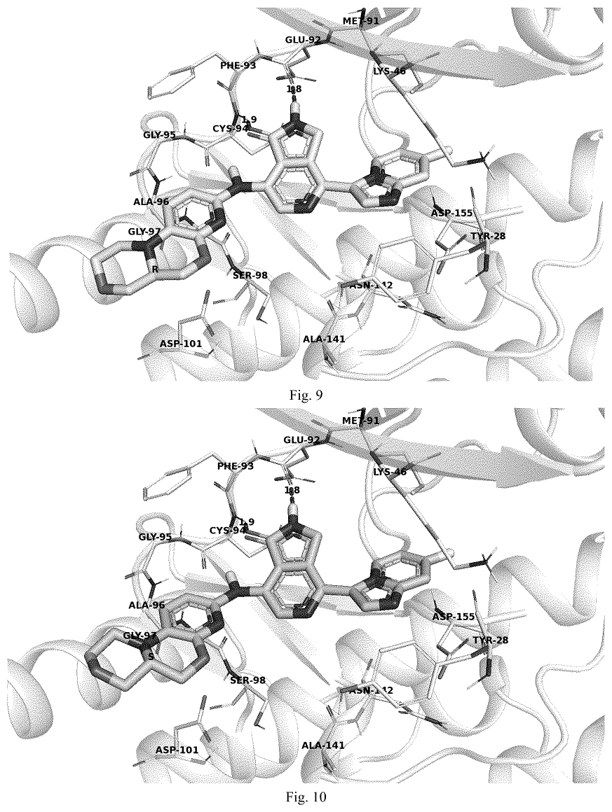

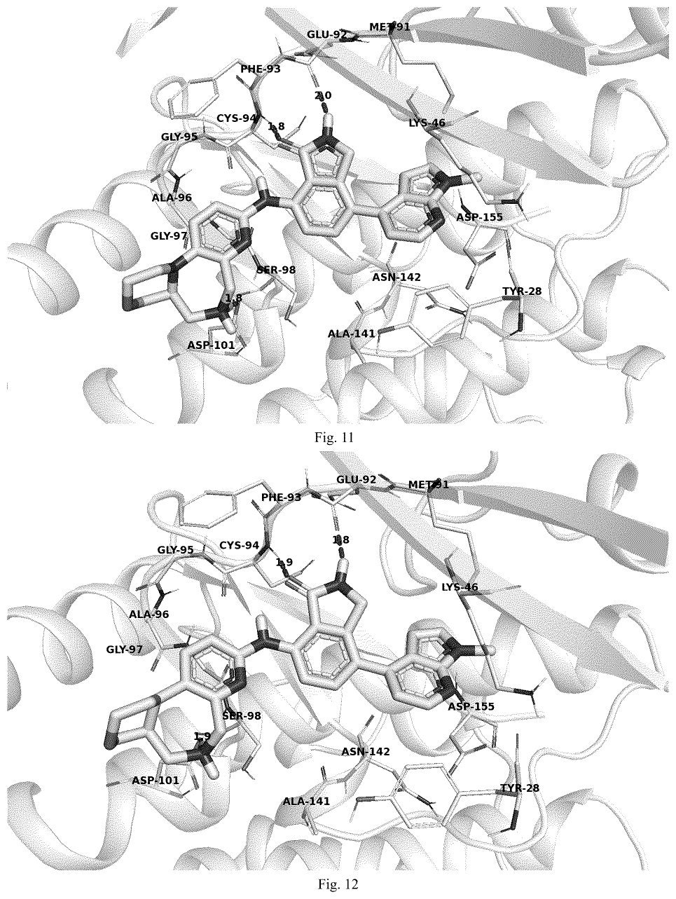

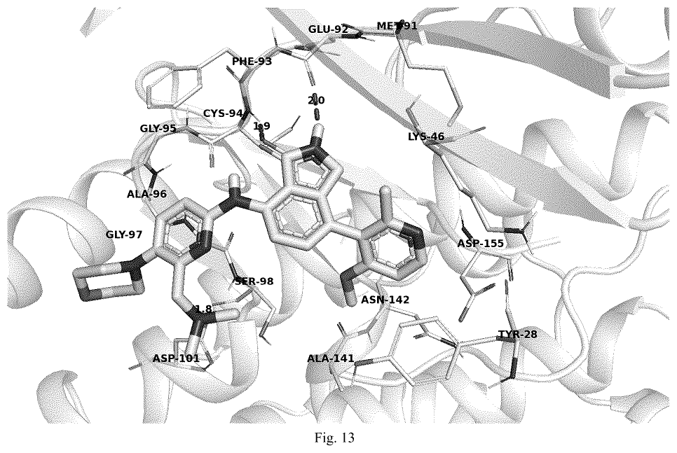

### DETAILED DESCRIPTION

The present disclosure is described in detail below by means of examples. However, it is not intended that these examples have any disadvantageous limitations to the present disclosure. Compounds disclosed herein can be prepared by a variety of synthetic methods well known to those skilled in the art, including the following enumerated embodiment, the embodiment formed by the following enumerated embodiment in combination with other chemical synthesis methods, and equivalent well known to those skilled in the art. Alternative embodiments include, but are not limited to examples disclosed herein. It will be apparent to those skilled in the art that various changes and modifications may be made to the embodiments disclosed herein without departing from the spirit and scope disclosed herein.

### Calculation Example 1

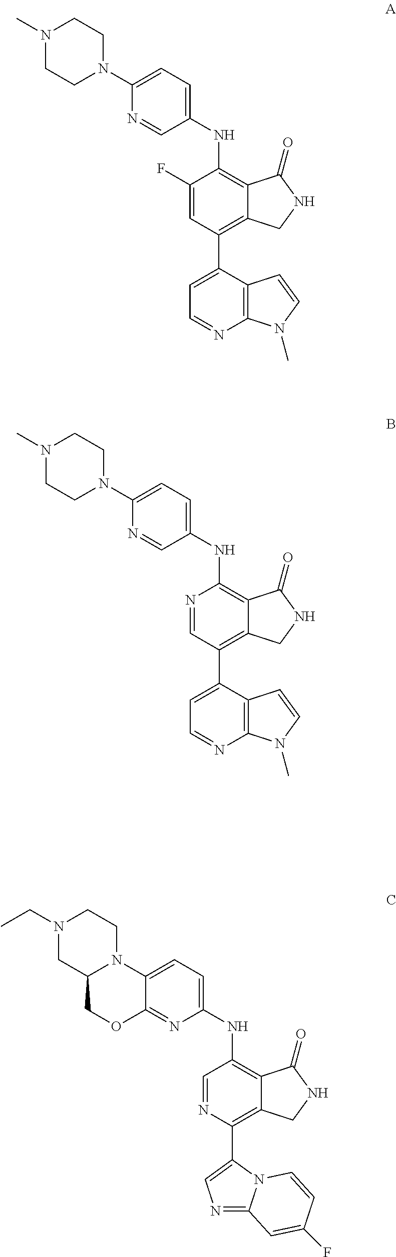 [SMILES: CN1CCN(c2ccc(Nc3c(F)cc(-c4ccnc5c4ccn5C)c4c3C(=O)NC4)cn2)CC1 ; CN1CCN(c2ccc(Nc3ncc(-c4ccnc5c4ccn5C)c4c3C(=O)NC4)cn2)CC1 ; CCN1CCN2c3ccc(Nc4cnc(-c5cnc6cc(F)ccn56)c5c4C(=O)NC5)nc3OC[C@H]2C1]

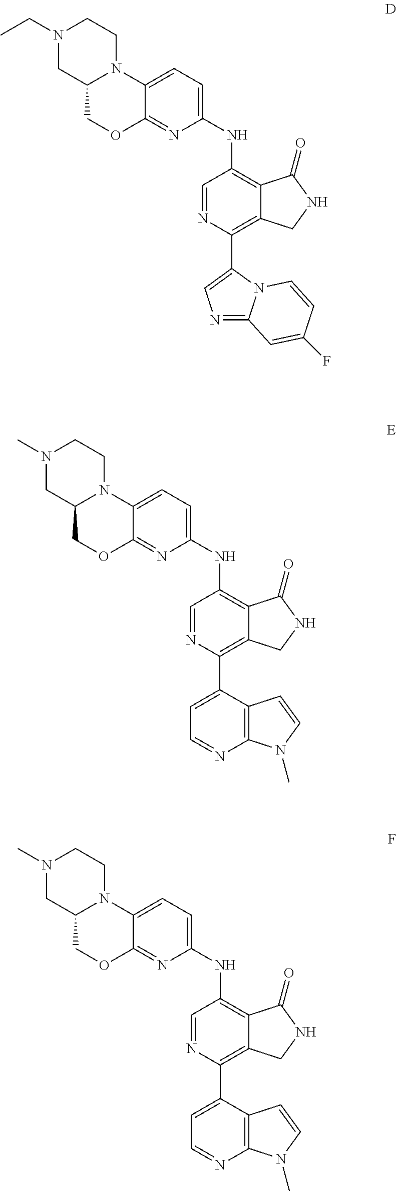 [SMILES: CCN1CCN2c3ccc(Nc4cnc(-c5cnc6cc(F)ccn56)c5c4C(=O)NC5)nc3OC[C@@H]2C1 ; CN1CCN2c3ccc(Nc4cnc(-c5ccnc6c5ccn6C)c5c4C(=O)NC5)nc3OC[C@H]2C1 ; CN1CCN2c3ccc(Nc4cnc(-c5ccnc6c5ccn6C)c5c4C(=O)NC5)nc3OC[C@@H]2C1]

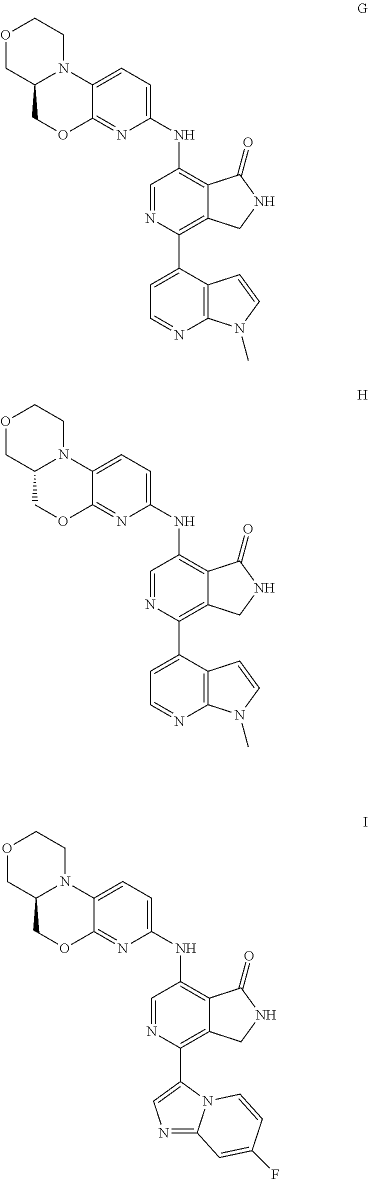 [SMILES: Cn1ccc2c(-c3ncc(Nc4ccc5c(n4)OC[C@H]4COCCN54)c4c3CNC4=O)ccnc21 ; Cn1ccc2c(-c3ncc(Nc4ccc5c(n4)OC[C@@H]4COCCN54)c4c3CNC4=O)ccnc21 ; O=C1NCc2c(-c3cnc4cc(F)ccn34)ncc(Nc3ccc4c(n3)OC[C@H]3COCCN43)c21]

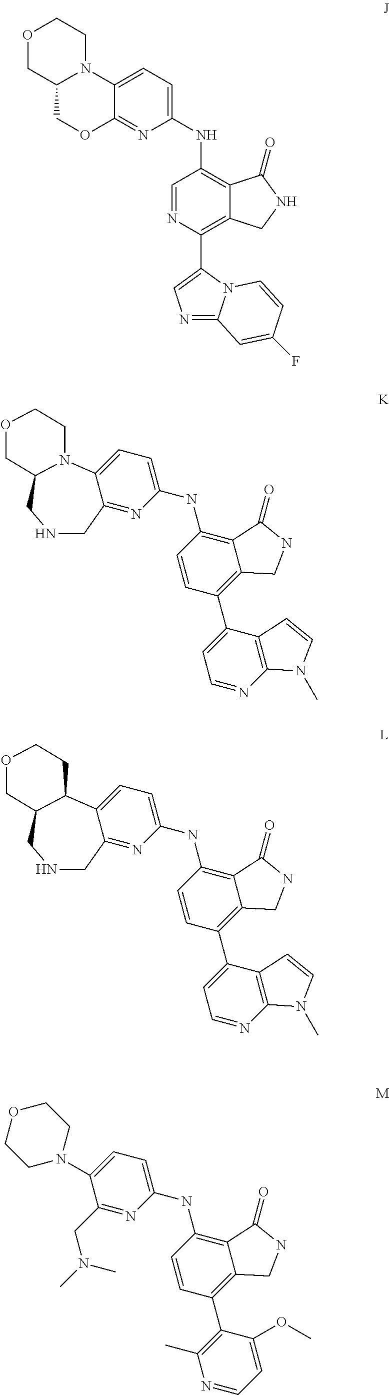 [SMILES: O=C1NCc2c(-c3cnc4cc(F)ccn34)ncc(Nc3ccc4c(n3)OC[C@@H]3COCCN43)c21 ; Cn1ccc2c(-c3ccc(Nc4ccc5c(n4)CNC[C@H]4COCCN54)c4c3CNC4=O)ccnc21 ; Cn1ccc2c(-c3ccc(Nc4ccc5c(n4)CNC[C@H]4COCC[C@@H]54)c4c3CNC4=O)ccnc21 ; COc1ccnc(C)c1-c1ccc(Nc2ccc(N3CCOCC3)c(CN(C)C)n2)c2c1CNC2=O]

The molecular docking process was performed by using Glide [1] in Maestro (Schrödinger version 2017-2) with default options. The co-crystal structure of HPK1 (PDB ID code: 7KAC) was selected as the docking template. To prepare the protein, hydrogen atoms were added using the protein preparation wizard module of Maestro [2], and the OPLS3 force field was used. For the preparation of ligand, a 3D structure was generated, and energy was minimized using LigPrep [3]. A 30 Å docking grid was generated using the center of mass of the ligand in the 7KAC crystal structure. The ligand was then removed and an example compound was placed in the molecular docking process. The interaction type between the protein receptor and the ligand was analyzed, and then the reasonable docking conformations were selected and saved based on the calculated docking score and the glide gscore value.
[1] Glide, Schrödinger, LLC, New York, NY, 2017.
[2] Maestro, Schrödinger, LLC, New York, NY, 2017.
[3] LigPrep, Schrödinger, LLC, New York, NY, 2017.

Conclusion: The compounds of the present disclosure have good binding to HPK1 protein.

### Example 1

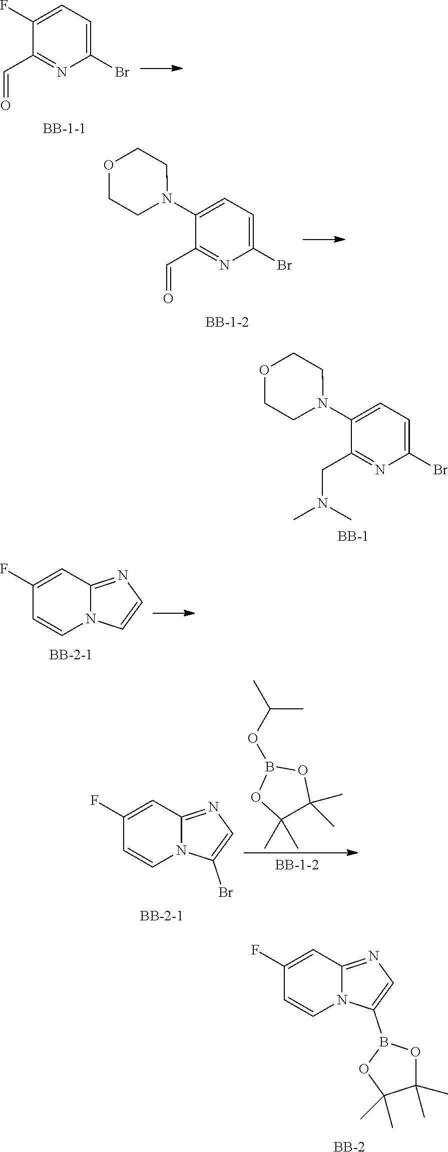

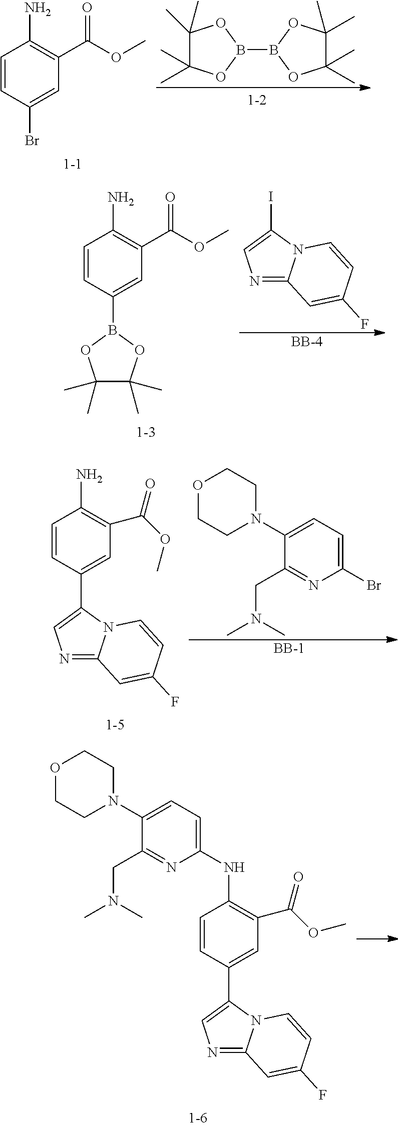

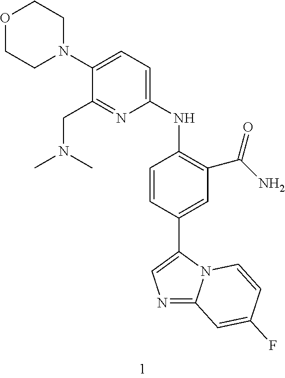 [SMILES: CN(C)Cc1nc(Nc2ccc(-c3cnc4cc(F)ccn34)cc2C(N)=O)ccc1N1CCOCC1]

### Step 1: Synthesis of Compound BB-1-2

Compound BB-1-1 (3 g, 14.71 mmol, 1 eq) was dissolved in DMF (30 mL) under nitrogen. Morpholine (2.56 g, 29.41 mmol, 2.59 mL, 2 eq) and potassium carbonate (4.06 g, 29.41 mmol, 2 eq) were added, and the mixture was reacted at 100° C. for 1 hour. The mixture was cooled, and water (100 mL) was added. The mixture was extracted with ethyl acetate (100 mL), dried over anhydrous sodium sulfate, filtered and concentrated to give a crude product. The crude product was purified by column chromatography (ethyl acetate/petroleum ether=50%) to give compound BB-1-2. MS m/z: 271.0, 273.0 [M+1]+; ¹H NMR (400 MHz, DMSO-d₆) δ 9.84 (s, 1H), 7.80-7.69 (m, 1H), 7.65-7.56 (m, 1H), 3.86-3.68 (m, 4H), 3.21-3.07 (m, 4H).

### Step 2: Synthesis of Compound BB-1

Compound BB-1-2 (2.7 g, 9.96 mmol, 1 eq) was dissolved in DCM (30 mL) under nitrogen. Dimethylamine aqueous solution (1.35 g, 11.95 mmol, 1.51 mL, 40% purity, 1.2 eq), glacial acetic acid (598.06 mg, 9.96 mmol, 569.58 μL, 1 eq), and NaBH(OAc)₃ (4.22 g, 19.92 mmol, 2 eq) were added, and the mixture was reacted at 20° C. for 1 hour. Water (50 mL) was added, and the pH was adjusted to 10 with sodium hydroxide aqueous solution (6M). The layers were separated. The organic phase was dried over anhydrous sodium sulfate, filtered and concentrated to give a crude product. The crude product was purified by column chromatography (ethyl acetate/petroleum ether=50%-methanol/ethyl acetate=0%-16%) to give compound BB-1. MS m/z: 300.1, 302.1 [M+1]⁺; ¹H NMR (400 MHz, DMSO-d₆) δ 7.54-7.42 (m, 2H), 3.79-3.68 (m, 4H), 3.45 (s, 2H), 3.04-2.92 (m, 4H), 2.23 (s, 6H).

### Step 3: Synthesis of Compound BB-2-2

Compound BB-2-1 (5 g, 36.73 mmol, 1 eq) was dissolved in DCM (50 mL) under nitrogen, and N-bromosuccinimide (7.19 g, 40.40 mmol, 1.1 eq) was added. The reaction solution was stirred at 20° C. for 2 hours. The reaction solution was poured into a saturated sodium thiosulfate solution (50 mL). The DCM was concentrated. Then, the mixture was slurried for 1 hour and filtered. The filter cake was washed with water (20 mL), and the filter cake was dried to give compound BB-2-2. MS m/z: 215.0 [M+1]⁺; ¹H NMR (400 MHz, CDCl₃) δ 8.11-8.08 (m, 1H), 7.59 (s, 1H), 7.29-7.27 (m, 1H), 6.86-6.82 (m, 1H).

### Step 4: Synthesis of Compound BB-2

Compound BB-2-2 (1.7 g, 7.91 mmol, 1 eq) and BB-2-3 (4.41 g, 23.72 mmol, 4.84 mL, 3 eq) were dissolved in tetrahydrofuran (35 mL) under nitrogen. A solution of isopropylmagnesium chloride in tetrahydrofuran (1.37 M, 11.54 mL, 2 eq) was added dropwise at 0° C., and the reaction solution was stirred at 0° C. for 2 hours. The reaction solution was poured into water (50 mL), and the aqueous phase was extracted with ethyl acetate (30 mL×2). The organic phases were combined, dried over anhydrous sodium sulfate, filtered and concentrated to give compound BB-2. MS m/z: 180.9 [M+1-82]⁺.

### Step 5: Synthesis of Compound 1-3

Compound 1-1 (5 g, 21.73 mmol, 1 eq) was dissolved in dioxane (50 mL) under nitrogen. 1-2 (8.28 g, 32.60 mmol, 1.5 eq), Pd(dppf)Cl₂ (1.59 g, 2.17 mmol, 0.1 eq), and potassium acetate (4.27 g, 43.47 mmol, 2 eq) were added, and the mixture was reacted at 90° C. for 1 hour. The mixture was cooled, and water (100 mL) was added to quench the reaction. The mixture was extracted with ethyl acetate (100 mL×2). The organic phases were combined, dried over anhydrous sodium sulfate, filtered and concentrated to give a crude product. The crude product was purified by column chromatography (ethyl acetate/petroleum ether=5%-16%) to give compound 1-3. MS m/z: 278.1 [M+1]⁺.

### Step 6: Synthesis of Compound 1-5

Compound 1-3 (500 mg, 1.80 mmol, 1 eq) was dissolved in dioxane (5 mL) and water (1 mL) under nitrogen. BB-4 (472.75 mg, 1.80 mmol, 1 eq), Pd(dppf)Cl₂ (132.02 mg, 180.43 μmol, 0.1 eq), and potassium carbonate (498.72 mg, 3.61 mmol, 2 eq) were added. The mixture was reacted at 90° C. for 2 hours. The mixture was cooled, and water (5 mL) was added to quench the reaction. The mixture was extracted with ethyl acetate (10 mL), dried over anhydrous sodium sulfate, filtered and concentrated to give a crude product. The crude product was purified by column chromatography (ethyl acetate/petroleum ether=50%) to give compound 1-5. MS m/z: 286.1 [M+1]⁺; ¹H NMR (400 MHz, DMSO-d₆) δ 8.47-8.30 (m, 1H), 7.87 (s, 1H), 7.69 (s, 1H), 7.52-7.38 (m, 2H), 7.04-6.87 (m, 4H), 3.81 (s, 3H).

### Step 7: Synthesis of Compound 1-6

Compound 1-5 (370 mg, 1.30 mmol, 1 eq) was dissolved in dioxane (10 mL) under nitrogen. BB-1 (389.35 mg, 1.30 mmol, 1 eq), cesium carbonate (845.18 mg, 2.59 mmol, 2 eq), Xantphos (225.14 mg, 389.10 μmol, 0.3 eq), and Pd₂(dba)₃ (237.54 mg, 259.40 μmol, 0.2 eq) were added. The mixture was reacted at 110° C. for 12 hours. The mixture was cooled, and water (20 mL) was added to quench the reaction. The mixture was extracted with ethyl acetate (20 mL), dried over anhydrous sodium sulfate, filtered and concentrated to give a crude product. The crude product was purified by column chromatography (ethyl acetate/methanol=10%-50%) to give compound 1-6. MS m/z: 505.2 [M+1]⁺.

### Step 8: Synthesis of Compound 1

Ammonium chloride (212.03 mg, 3.96 mmol, 10 eq) was added to a solution of lithium bis(trimethylsilyl)amide in tetrahydrofuran (1 M, 3.96 mL, 10 eq) under nitrogen, and compound 1-6 (200 mg, 396.39 μmol, 1 eq) dissolved in tetrahydrofuran (2 mL) was added. The mixture was reacted at 20° C. for 1 hour. Methanol (5 mL) was added to quench the reaction, and the reaction solution was concentrated to give a crude product. The crude product was purified by prep-HPLC (chromatography column: Phenomenex C18 75*30 mm*3 μm; mobile phase: [water (NH₄HCO₃)-acetonitrile]; acetonitrile %: 15%-55%, 8 min) to give compound 1. MS m/z: 490.3 [M+1]⁺; ¹H NMR (400 MHz, DMSO-d₆) δ 11.11 (s, 1H), 8.87-8.73 (m, 1H), 8.71-8.56 (m, 1H), 8.28 (s, 1H), 7.97 (s, 1H), 7.74 (s, 1H), 7.71-7.61 (m, 2H), 7.58-7.46 (m, 2H), 7.04-6.94 (m, 1H), 6.91-6.82 (m, 1H), 3.79-3.71 (m, 4H), 3.53 (s, 2H), 3.01-2.91 (m, 4H), 2.29 (s, 6H).

### Example 2

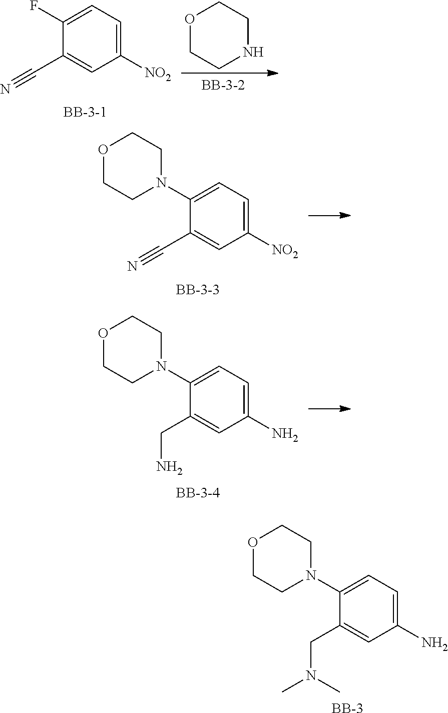

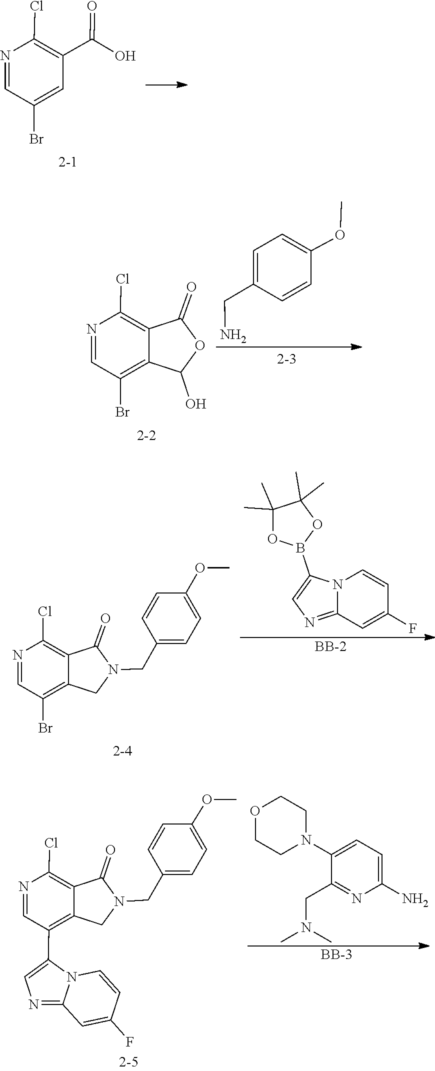

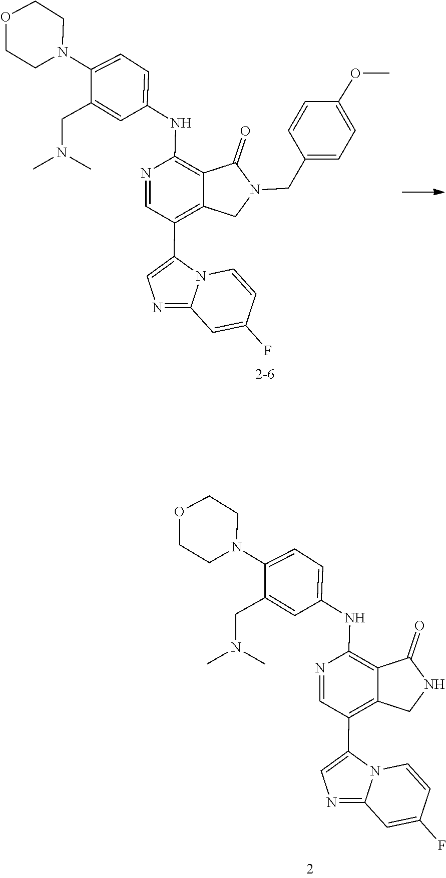 [SMILES: COc1ccc(CN2Cc3c(-c4cnc5cc(F)ccn45)cnc(Nc4ccc(N5CCOCC5)c(CN(C)C)c4)c3C2=O)cc1 ; CN(C)Cc1cc(Nc2ncc(-c3cnc4cc(F)ccn34)c3c2C(=O)NC3)ccc1N1CCOCC1]

### Step 1: Synthesis of Compound BB-3-3

Compound BB-3-1 (4.7 g, 28.29 mmol, 1 eq) and BB-3-2 (2.71 g, 31.12 mmol, 2.74 mL, 1.1 eq) were dissolved in DMF (50 mL) under nitrogen, and the reaction solution was stirred at 20° C. for 6 minutes. The reaction solution was poured into water (500 mL), and the mixture was stirred for 10 minutes. Then, the mixture was filtered, and the filter cake was washed with ethanol (30 mL). The filter cake was dried to give compound BB-3-3. MS m/z: 234.2 [M+1]⁺; ¹H NMR (400 MHz, CDCl₃) δ 8.46 (d, J=2.8 Hz, 1H), 8.32-8.29 (m, 1H), 7.00 (d, J=2.0 Hz, 1H), 3.93-3.91 (m, 4H), 3.51-3.49 (m, 4H).

### Step 2: Synthesis of Compound BB-3-4

Raney nickel (2 g, 34.08 mmol, 3.97 eq) was added to a dry hydrogenation bottle with argon. Tetrahydrofuran (20 mL) was slowly added along the wall of the bottle, and aqueous ammonia (7.28 g, 51.93 mmol, 8.00 mL, 25% purity, 6.06 eq) was added. Then, BB-3-3 (2 g, 8.58 mmol, 1 eq) was added, and the reaction system was purged with hydrogen 3 times. The reaction solution was stirred at 30° C. and under hydrogen (pressure of 15 Psi) for 3 hours. The reaction solution was filtered, and the filter cake was washed with methanol (50 mL). The filtrate was concentrated to give compound BB-3-4. MS m/z: 208.3 [M+1]⁺; ¹H NMR (400 MHz, CDCl₃) δ 6.99 (d, J=8.4 Hz, 1H), 6.65 (d, J=2.8 Hz, 1H), 6.57 (d, J=8.0 Hz, 1H), 3.82 (s, 6H), 3.58 (s, 2H), 2.85-2.83 (m, 4H), 1.58 (m, 2H).

### Step 3: Synthesis of Compound BB-3

Compound BB-3-4 (0.5 g, 2.41 mmol, 1 eq) and an aqueous solution of formaldehyde (391.52 mg, 4.82 mmol, 359.19 μL, 37% purity, 2 eq) were added to methanol (20 mL) under nitrogen. Zinc chloride (164.39 mg, 1.21 mmol, 56.49 μL, 0.5 eq) and sodium cyanoborohydride (151.59 mg, 2.41 mmol, 1 eq) were added. The mixture was reacted at 25° C. for 12 hours. The reaction solution was added to water (30 mL), and the mixture was extracted with dichloromethane:methanol (10:1, 30 mL×4). Then, the organic phases were combined, dried over anhydrous sodium sulfate, and filtered. The filtrate was concentrated under reduced pressure to give compound BB-3. MS m/z: 236.1 [M+1]⁺.

### Step 4: Synthesis of Compound 2-2

A solution of n-butyllithium (2.5 M, 44.32 mL, 2.62 eq) in tetrahydrofuran was added to tetrahydrofuran (120 mL) at −78° C. under nitrogen, and 2,2,6,6-tetramethylpiperidine (17.92 g, 126.88 mmol, 21.54 mL, 3 eq) was added dropwise. The mixture was warmed to 0° C. and stirred for 1 hour. The mixture was cooled to −78° C., and 2-1 (10 g, 42.29 mmol, 1 eq) dissolved in tetrahydrofuran (80 mL) was added dropwise. The mixture was stirred for 1.5 hours, and then DMF (11.87 g, 162.46 mmol, 12.50 mL, 3.84 eq) was added. The mixture was warmed to 20° C. and stirred for 4 hours. The reaction solution was poured into an aqueous solution of hydrochloric acid (50 mL, 1M) to quench the reaction. The aqueous phase was extracted with ethyl acetate (50 mL×2). The organic phases were combined, dried over anhydrous sodium sulfate, filtered and concentrated to give a crude product. The crude product was purified by column chromatography (ethyl acetate:petroleum ether=10%-33%) to give compound 2-2. MS m/z: 263.9 [M+1]⁺; ¹H NMR (400 MHz, DMSO-d₆) δ 8.92 (s, 1H), 8.55 (d, J=8.0 Hz, 1H), 6.61 (d, J=5.2 Hz, 1H).

### Step 5: Synthesis of Compound 2-4

Compound 2-2 (2 g, 7.56 mmol, 1 eq) and compound 2-3 (1.09 g, 7.94 mmol, 1.03 mL, 1.05 eq) were dissolved in DCM (50 mL) under nitrogen. Glacial acetic acid (45.41 mg, 756.26 μmol, 43.25 μL, 0.1 eq) was added, and the mixture was stirred at 20° C. for 1 hour. NaBH(OAc)₃ (3.21 g, 15.13 mmol, 2 eq) was added, and the mixture was stirred for another 15 hours. The reaction solution was poured into a saturated sodium bicarbonate solution (100 mL), and the dichloromethane was concentrated. The aqueous phase was slurried for 30 minutes and then filtered. Anhydrous ethanol (30 mL) was added to the filter cake. The mixture was slurried for 30 minutes and then filtered. The filter cake was concentrated and dried to give compound 2-4. MS m/z: 367.0 [M+1]⁺; ¹H NMR (400 MHz, DMSO-d₆) δ 8.72 (s, 1H), 7.27 (d, J=8.8 Hz, 2H), 6.92 (d, J=8.4 Hz, 2H), 4.65 (s, 2H), 4.34 (s, 2H), 3.73 (s, 3H).

### Step 6: Synthesis of Compound 2-5

Compound 2-4 (500 mg, 1.36 mmol, 1 eq) and BB-2 (2 g, 2.29 mmol, 30% purity, 1.68 eq) were dissolved in dioxane (30 mL) and water (5 mL) under nitrogen. Potassium phosphate (866.11 mg, 4.08 mmol, 3 eq) and Pd(dppf)C₂ (99.52 mg, 136.01 μmol, 0.1 eq) were added, and the reaction solution was stirred at 50° C. for 12 hours. The reaction solution was concentrated, and ethyl acetate (50 mL) and water (20 mL) were added. The layers were separated. The aqueous phase was extracted with ethyl acetate (10 mL×2). The organic phases were combined, dried over anhydrous sodium sulfate, filtered and concentrated. The crude product was purified by column chromatography (ethyl acetate/petroleum ether=10%-100%) to give compound 2-5. MS m/z:423.1 [M+1]⁺; ¹H NMR (400 MHz, CDCl₃) δ 8.65 (s, 1H), 8.04 (d, J=5.2 Hz, 1H), 7.67 (s, 1H), 7.35 (d, J=9.2 Hz, 1H), 7.24 (d, J=8.8 Hz, 2H), 6.86-6.80 (m, 3H), 4.72 (s, 2H), 4.22 (s, 2H), 3.78 (s, 3H).

### Step 7: Synthesis of Compound 2-6

Compound 2-5 (150 mg, 354.75 μmol, 1 eq) and BB-3 (83.48 mg, 354.75 μmol, 1 eq) were dissolved in DCM (10 mL) under nitrogen. RuPhos Pd G2 (27.55 mg, 35.47 μmol, 0.1 eq), Ruphos (16.55 mg, 35.47 μmol, 0.1 eq) and cesium carbonate (346.75 mg, 1.06 mmol, 3 eq) were added. The mixture was stirred at 110° C. for 1 hour. The reaction solution was filtered, and the filtrate was concentrated to give a crude product. The crude product was purified by column chromatography (dichloromethane/methanol 0%-16%) to give compound 2-6. MS m/z: 622.3 [M+1]⁺; ¹H NMR (400 MHz, DMSO-d₆) δ 9.20 (s, 1H), 8.48-8.43 (m, 2H), 7.75 (m, 1H), 7.65 (s, 1H), 7.51 (s, 1H), 7.26-7.24 (m, 2H), 7.16 (s, 1H), 6.96-6.95 (m, 1H), 6.89-6.85 (m, 2H), 4.62 (s, 2H), 4.38 (s, 2H), 3.75-3.69 (m, 9H), 2.88-2.86 (m, 4H), 2.28-2.24 (m, 6H).

### Step 8: Synthesis of Compound 2

Compound 2-6 (130 mg, 209.10 μmol, 1 eq) was dissolved in trifluoroacetic acid (10 mL), and the reaction solution was stirred at 130° C. under microwave for 16 hours. The reaction solution was concentrated and purified by prep-HPLC (chromatography column: Waters Xbridge BEH C18 100*30 mm*10 μm; mobile phase: [water (NH₄HCO₃)-acetonitrile]; acetonitrile %: 10%-40%, 8 min) to give compound 2. MS m/z: 502.1 [M+1]⁺; ¹H NMR (400 MHz, DMSO-d₆) δ 9.22 (s, 1H), 8.91 (s, 1H), 8.51-8.48 (m, 2H), 7.87-7.86 (m, 1H), 7.80 (s, 1H), 7.61-7.60 (m, 1H), 7.54-7.51 (m, 1H), 7.14-7.12 (m, 1H), 6.97-6.96 (m, 1H), 4.40 (s, 2H), 3.74-3.72 (m, 4H), 3.48-3.43 (m, 2H), 2.88-2.86 (m, 4H), 2.22-2.18 (m, 6H).

### Example 3

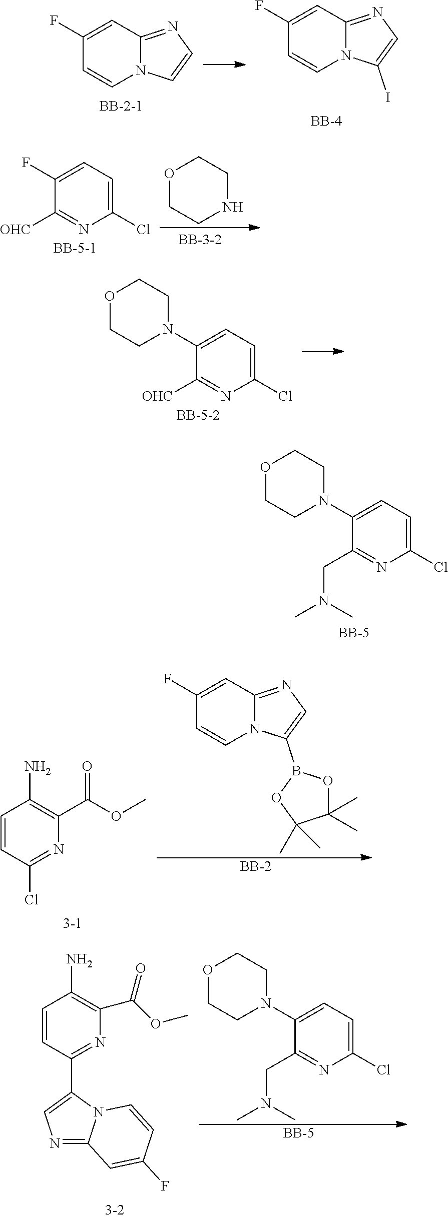

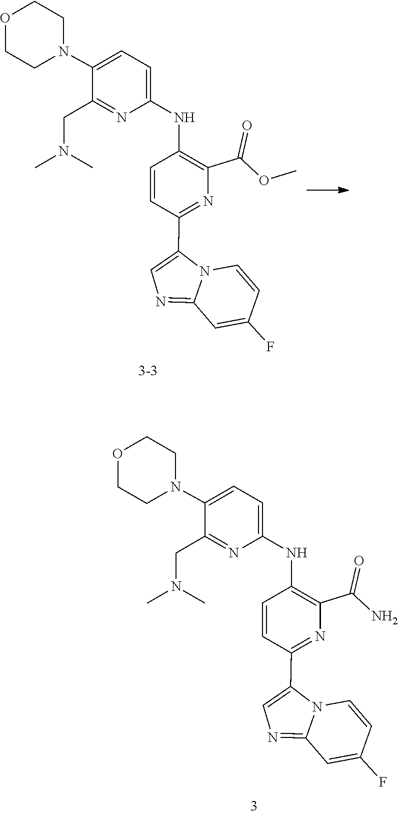 [SMILES: COC(=O)c1nc(-c2cnc3cc(F)ccn23)ccc1Nc1ccc(N2CCOCC2)c(CN(C)C)n1 ; CN(C)Cc1nc(Nc2ccc(-c3cnc4cc(F)ccn34)nc2C(N)=O)ccc1N1CCOCC1]

### Step 1: Synthesis of Compound BB-4

Compound BB-2-1 (4.26 g, 31.29 mmol, 1 eq) was dissolved in anhydrous N, N-dimethylformamide (40 mL) at 20° C. under nitrogen, and N-iodosuccinimide (7.74 g, 34.42 mmol, 1.1 eq) was added. The mixture was heated to 50° C. and reacted for 1 hour. A saturated sodium thiosulfate solution (40 mL) was added to quench the reaction. The mixture was extracted with ethyl acetate (40 mL×3). The organic phases were combined, washed with water (100 mL×2) and saturated brine (50 mL×2) successively, dried over anhydrous sodium sulfate, vacuum filtered and concentrated under reduced pressure to give compound BB-4. MS m/z: 263.0 [M+1]⁺; ¹H NMR (400 MHz, DMSO-d₆) δ 8.40-8.37 (m, 1H), 7.69 (s, 1H), 7.55-7.52 (m, 1H), 7.11-7.07 (m, 1H).

### Step 2: Synthesis of Compound BB-5-2

Compound BB-5-1 (2.5 g, 15.67 mmol, 1 eq) was dissolved in N, N-dimethylformamide (25 mL) at 20° C. under nitrogen. Morpholine (2.73 g, 31.34 mmol, 2.76 mL, 2 eq) and potassium carbonate (6.50 g, 47.01 mmol, 3 eq) were added. The mixture was heated to 100° C. and reacted for 2 hours. Water (30 mL) was added to quench the reaction. The mixture was extracted with ethyl acetate (30 mL×3). The organic phases were combined, washed with saturated brine (20 mL), dried over anhydrous sodium sulfate, and filtered. The filtrate was concentrated under reduced pressure. The resulting crude product was purified by column chromatography (ethyl acetate/petroleum ether=0%-20%) to give compound BB-5-2. MS m/z: 227.0 [M+1]⁺; ¹H NMR (400 MHz, DMSO-d₆) δ 9.87 (s, 1H), 7.72-7.70 (m, 1H), 7.66-7.64 (m, 1H), 3.77-3.75 (m, 4H), 3.13-3.11 (m, 4H).

### Step 3: Synthesis of Compound BB-5

Compound BB-5-2 (1 g, 4.41 mmol, 1 eq) was dissolved in methanol (20 mL) at 0° C. under nitrogen. An aqueous solution of dimethylamine (1.49 g, 13.24 mmol, 1.68 mL, 40% purity, 3 eq) and glacial acetic acid (105.98 mg, 1.76 mmol, 100.93 μL, 0.4 eq) were added. The mixture was reacted at 0° C. for 0.5 hours, and then sodium cyanoborohydride (1.11 g, 17.65 mmol, 4 eq) was added. The mixture was reacted at 0° C. for 1.5 hours. Water (20 mL) was added to quench the reaction, and the solvent was concentrated under reduced pressure. The mixture was extracted with ethyl acetate (20 mL×3). The organic phases were combined, washed with saturated brine (10 mL), dried over anhydrous sodium sulfate, and filtered. The filtrate was concentrated under reduced pressure, and the resulting crude product was purified by column chromatography (ethyl acetate/petroleum ether=0%-100%) to give compound BB-5. MS m/z: 256.2 [M+1]⁺; ¹H NMR (400 MHz, DMSO-d₆) δ 7.57 (d, J=8.4 Hz, 1H), 7.38 (d, J=8.4 Hz, 1H), 3.76-3.73 (m, 4H), 3.45 (s, 2H), 3.03-2.97 (m, 4H), 2.22 (s, 6H).

### Step 4: Synthesis of Compound 3-2

Compound 3-1 (300 mg, 1.61 mmol, 1 eq) was dissolved in dioxane (15 mL) and water (3 mL) under nitrogen. Compound BB-2 (4.6 g, 2.28 mmol, 13% purity, 1.42 eq), potassium phosphate (1.02 g, 4.82 mmol, 3 eq), and XPhos Pd G2 (126.50 mg, 160.78 μmol, 0.1 eq) were added. The mixture was reacted at 100° C. for 2 hours. The mixture was cooled, and water (10 mL) was added to quench the reaction. The mixture was extracted with ethyl acetate (20 mL×3). The organic phases were combined, washed with saturated brine (10 mL), dried over anhydrous sodium sulfate, vacuum filtered, and concentrated under reduced pressure. The resulting crude product was purified by column chromatography (ethyl acetate/petroleum ether=0%-100%) to give compound 3-2. MS m/z: 287.2 [M+1]⁺; ¹H NMR (400 MHz, DMSO-d₆) δ 10.24-10.15 (m, 1H), 8.25 (s, 1H), 7.97 (d, J=8.8 Hz, 1H), 7.53-7.52 (m, 1H), 7.42 (d, J=2.4 Hz, 1H), 7.22-7.17 (m, 1H), 6.88 (s, 2H), 3.92 (s, 3H).

### Step 5: Synthesis of Compound 3-3

Compound 3-2 (50 mg, 174.67 μmol, 1 eq) was dissolved in dioxane (2 mL) under nitrogen. Compound BB-5 (53.60 mg, 209.60 μmol, 1.2 eq), cesium carbonate (113.82 mg, 349.33 μmol, 2 eq), BINAP (13.05 mg, 20.96 μmol, 0.12 eq), and palladium acetate (3.92 mg, 17.47 μmol, 0.1 eq) were added. The mixture was reacted at 120° C. for 2 hours. The mixture was cooled, and water (10 mL) was added to quench the reaction. The mixture was extracted with ethyl acetate (10 mL×3). The organic phases were combined, washed with saturated brine (10 mL), dried over anhydrous sodium sulfate, and filtered. The filtrate was concentrated under reduced pressure. The resulting crude product was purified by a preparative thin layer chromatography plate (ethyl acetate/methanol/triethylamine=80:20:0.5%) to give compound 3-3. MS m/z: 506.3 [M+1]⁺; ¹H NMR (400 MHz, DMSO-d₆) δ 10.22-10.18 (m, 1H), 9.24-9.13 (m, 1H), 8.41 (s, 1H), 8.20 (d, J=9.2 Hz, 1H), 7.63-7.59 (m, 2H), 7.27-7.24 (m, 1H), 7.23-7.05 (m, 1H), 4.01 (s, 3H), 3.75-3.69 (m, 9H), 2.95-2.93 (m, 4H), 2.40-2.38 (m, 4H).

### Step 6: Synthesis of Compound 3

A solution of lithium bis(trimethylsilyl)amide in tetrahydrofuran (1 M, 427.26 μL, 8 eq) was added to a mixture of compound 3-3 (27 mg, 53.41 μmol, 1 eq) and ammonium chloride (17.14 mg, 320.45 μmol, 6 eq) under nitrogen. The resulting mixture was reacted at 20° C. for 1 hour. Methanol (2 mL) was added to quench the reaction. The solvent was concentrated under reduced pressure to give a crude product. The crude product was purified by prep-HPLC (chromatography column: Waters Xbridge BEH C18 100*30 mm*110 μm; mobile phase: [water (NH₄HCO₃)-acetonitrile]; acetonitrile %: 15%-45%, 8 min) to give compound 3. MS m/z: 491.2 [M+1]⁺; ¹H NMR (400 MHz, DMSO-d₆) δ 11.56 (s, 1H), 9.59-9.56 (m, 1H), 9.36 (d, J=8.8 Hz, 1H), 8.29 (s, 1H), 8.22-8.20 (m, 1H), 8.08 (d, J=10.0 Hz, 1H), 8.01-7.99 (m, 1H), 7.63-7.49 (m, 2H), 7.19-7.06 (m, 1H), 6.90 (d, J=8.4 Hz, 1H), 3.76-3.73 (m, 4H), 3.54 (s, 2H), 2.96-2.94 (m, 4H), 2.30 (s, 6H).

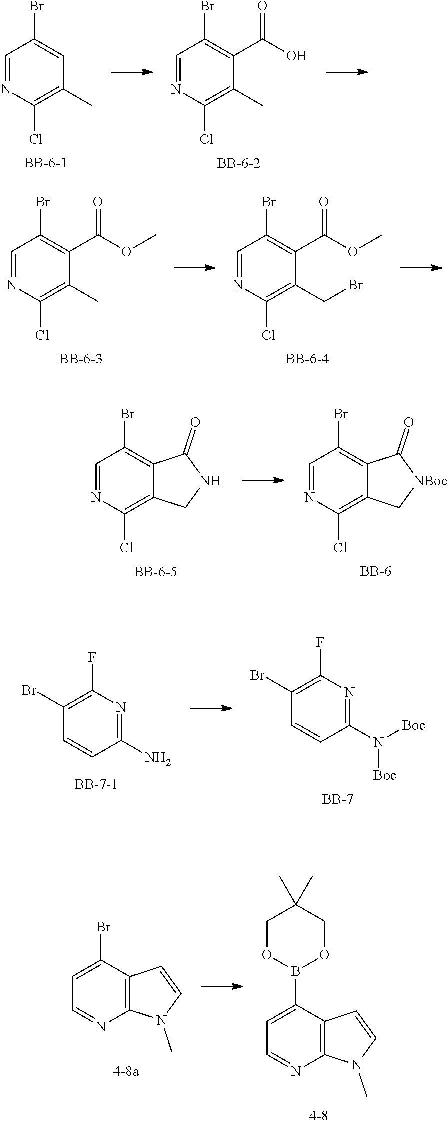

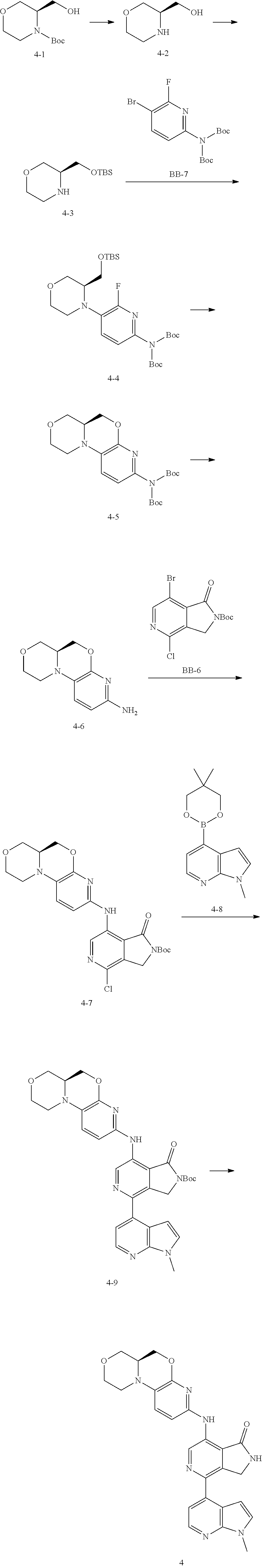

### Step 1: Synthesis of Compound BB-6-2

Compound BB-6-1 (0.5 g, 2.42 mmol, 1 eq) was dissolved in tetrahydrofuran (5 mL) under nitrogen, and the resulting mixture was cooled to −78° C. A solution of lithium diisopropylamide in tetrahydrofuran (2 M, 1.45 mL, 1.2 eq) was added, and the resulting mixture was reacted at −78° C. for 1 hour. Carbon dioxide (1.07 g, 24.22 mmol, 10 eq) was introduced, and the mixture was warmed to 25° C. and reacted for 1 hour. Similarly, compound BB-6-1 (8 g, 38.75 mmol, 1 eq) was dissolved in tetrahydrofuran (80 mL) under nitrogen, and the resulting mixture was cooled to −78° C. A solution of lithium diisopropylamide in tetrahydrofuran (2 M, 23.25 mL, 1.2 eq) was added, and the resulting mixture was reacted at −78° C. for 1 hour. Carbon dioxide was introduced (17.05 g, 387.47 mmol, 10 eq), and the mixture was warmed to 25° C. and reacted for 1 hour. The reaction solutions were combined, and a saturated sodium bicarbonate solution (50 mL) was added to quench the reaction. The mixture was extracted with ethyl acetate (50 mL×2), and the organic phase was discarded. The pH of the aqueous phase was adjusted to 4 with hydrochloric acid (3 M, about 50 mL), and the resulting mixture was extracted with dichloromethane:methanol (10:1, 100 mL×5). The organic phases were combined, dried over anhydrous sodium sulfate, filtered and concentrated to give compound BB-6-2. MS m/z: 250.1, 252.1 [M+1]⁺; ¹H NMR (400 MHz, DMSO-d₆) δ 8.47 (s, 1H), 2.30 (s, 3H).

### Step 2: Synthesis of Compound BB-6-3

Compound BB-6-2 (2.7 g, 10.78 mmol, 1 eq) was dissolved in DMF (27 mL) under nitrogen. Potassium carbonate (4.47 g, 32.34 mmol, 3 eq) and iodomethane (3.06 g, 21.56 mmol, 1.34 mL, 2 eq) were added. The resulting mixture was reacted at 25° C. for 2 hours. Water (30 mL) was added, and the resulting mixture was extracted with ethyl acetate (50 mL×2). The organic phases were combined, washed with saturated brine (30 mL×2), dried over anhydrous sodium sulfate, filtered and concentrated to give a crude product. The crude product was purified by column chromatography (ethyl acetate/petroleum ether=5%-10%) to give compound BB-6-3. MS m/z: 266.0, 263.9 [M+1]⁺; ¹H NMR (400 MHz, DMSO-d₆) δ 8.59 (s, 1H), 3.97 (s, 3H), 2.31 (s, 3H).

### Step 3: Synthesis of Compound BB-6-4

Compound BB-6-3 (0.97 g, 3.67 mmol, 1 eq) was dissolved in 1,2-dichloroethane (10 mL) under nitrogen. Azobisisobutyronitrile (60.22 mg, 366.72 μmol, 0.1 eq) and N-bromosuccinimide (1.31 g, 7.33 mmol, 2 eq) were added. The resulting mixture was reacted at 85° C. for 2 hours. The mixture was cooled, and the reaction solution was concentrated to give a crude product. The crude product was purified by column chromatography (ethyl acetate/petroleum ether=5%) to give compound BB-6-4. MS m/z: 345.8, 343.8 [M+1]⁺; ¹H NMR (400 MHz, DMSO-d₆) δ 8.76 (s, 1H), 4.60 (s, 2H), 4.00 (s, 3H).

### Step 4: Synthesis of Compound BB-6-5

Compound BB-6-4 (0.6 g, 1.75 mmol, 1 eq) was dissolved in methanol (6 mL) under nitrogen, and a solution of ammonia (7 M, 6.00 mL, 24.04 eq) in methanol was added. The resulting mixture was reacted at 25° C. for 1 hour. The mixture was filtered, and the filter cake was washed with methanol (5 mL). The filter cake was dried to give compound BB-6-5. MS m/z: 246.9, 248.9 [M+1]⁺; ¹H NMR (400 MHz, DMSO-d₆) δ 9.36 (s, 1H), 8.73 (s, 1H), 4.47 (s, 2H).

### Step 5: Synthesis of Compound BB-6

Compound BB-6-5 (0.7 g, 2.83 mmol, 1 eq) was dissolved in dioxane (7 mL) under nitrogen. Di-tert-butyl dicarbonate (925.99 mg, 4.24 mmol, 974.72 μL, 1.5 eq) and 4-dimethylaminopyridine (69.11 mg, 565.71 μmol, 0.2 eq) were added. The resulting mixture was reacted at 25° C. for 1 hour. The reaction solution was concentrated to give a crude product. The crude product was purified by column chromatography (ethyl acetate/petroleum ether=0%-5%) to give compound BB-6. MS m/z: 346.9, 348.9 [M+1]⁺; ¹H NMR (400 MHz, DMSO-d₆) δ 8.75 (s, 1H), 4.74 (s, 2H), 1.54 (s, 9H).

### Step 6: Synthesis of Compound BB-7

Compound BB-7-1 (2.6 g, 13.61 mmol, 1 eq) was dissolved in tetrahydrofuran (55 mL) under nitrogen, and the resulting mixture was cooled to 0° C. Then, a solution of sodium bis(trimethylsilyl)amide in tetrahydrofuran (1 M, 27.23 mL, 2 eq) was added, and the resulting mixture was stirred for 0.5 hours. Then, di-tert-butyl dicarbonate (7.43 g, 34.03 mmol, 7.82 mL, 2.5 eq) was further added, and the resulting mixture was reacted at 25° C. for 2 hours. The reaction solution was slowly added to a saturated aqueous solution of ammonium chloride (100 mL). The mixture was extracted with ethyl acetate (100 mL×3). Then, the organic phases were combined, dried over anhydrous sodium sulfate, and filtered. The filtrate was concentrated under reduced pressure to give a crude product. The crude product was purified by column chromatography (ethyl acetate/petroleum ether=0%-2%) to give compound BB-7. ¹H NMR (400 MHz, CDCl₃) δ ppm 7.99-7.95 (m, 1H) 7.14 (m, 1H) 1.48 (s, 18H).

### Step 7: Synthesis of Compound 4-2

Compound 4-1 (5 g, 23.01 mmol, 1 eq) was added to hydrochloric acid/methanol (100 mL) under nitrogen, and the resulting mixture was reacted at 25° C. for 2 hours. The reaction solution was concentrated under reduced pressure to give a hydrochloride of compound 4-2, and the hydrochloride was dissolved in methanol (10 mL) and water (10 mL). Aqueous ammonia (2 mL) was added dropwise to adjust the pH to 10. The mixture was extracted with dichloromethane (50 mL×2). The organic phases were combined, dried over anhydrous sodium sulfate, filtered and concentrated to give compound 4-2. ¹H NMR (400 MHz, CD₃OD) δ ppm 4.03-3.99 (m, 2H), 3.80-3.76 (m, 2H), 3.71-3.69 (m, 2H), 3.33 (s, 1H), 3.30-3.21 (m, 2H).

### Step 8: Synthesis of Compound 4-3

Compound 4-2 (2.7 g, 23.05 mmol, 1 eq) was dissolved in DCM (54 mL) under nitrogen. Triethylamine (4.66 g, 46.10 mmol, 6.42 mL, 2 eq), TBSCl (3.47 g, 23.05 mmol, 2.82 mL, 1 eq) and imidazole (1.88 g, 27.66 mmol, 1.2 eq) were added. The resulting mixture was reacted at 25° C. for 2 hours. The reaction solution was added to a saturated aqueous solution of ammonium chloride (50 mL), and the mixture was extracted with dichloromethane (50 mL×3). Then, the organic phases were combined, dried over anhydrous sodium sulfate, and filtered. The filtrate was concentrated under reduced pressure. The resulting crude product was purified by column chromatography (dichloromethane/methanol=0-1%) to give compound 4-3. ¹H NMR (400 MHz, CD₃OD) δ ppm 3.81 (m, 1H) 3.74 (m, 1H) 3.56-3.54 (m, 2H) 3.27-3.26 (m, 1H) 3.24 (dd, J=11.2, 9.8 Hz, 1H) 2.88-2.85 (m, 3H) 0.92-0.91 (m, 9H) 0.08 (s, 6H).

### Step 9: Synthesis of Compound 4-4

Compound 4-3 (1.6 g, 6.91 mmol, 1 eq) and compound BB-7 (2.71 g, 6.91 mmol, 1 eq) were added to toluene (32 mL) under nitrogen. Then, cesium carbonate (4.51 g, 13.83 mmol, 2 eq), Ruphos (645.29 mg, 1.38 mmol, 0.2 eq), and tris(dibenzylideneacetone)dipalladium(0) (949.73 mg, 1.04 mmol, 0.15 eq) were added. The resulting mixture was reacted at 100° C. for 16 hours. The reaction solution was added to water (50 mL). Then, the mixture was extracted with ethyl acetate (50 mL×3). Then, the organic phases were combined, dried over anhydrous sodium sulfate, and filtered. The filtrate was concentrated under reduced pressure to give a crude product. The crude product was separated by column chromatography (ethyl acetate/petroleum ether=0%-4%) to give compound 4-4.

### Step 10: Synthesis of Compound 4-5

Compound 4-4 (0.8 g, 1.48 mmol, 1 eq) was added to tetrahydrofuran (45 mL) under nitrogen, and a solution of tetrabutylammonium fluoride in tetrahydrofuran (1 M, 3.69 mL, 2.5 eq) was added. The resulting mixture was reacted at 50° C. for 3 hours. The reaction solution was concentrated under reduced pressure. The resulting crude product was purified by column chromatography (ethyl acetate/petroleum ether=0%-30%) to give compound 4-5. MS m/z: 408.1 [M+1]⁺; ¹H NMR (400 MHz, CDCl₃) δ ppm 7.02 (d, J=8.2 Hz, 1H), 6.76 (d, J=8.2 Hz, 1H), 4.34-4.32 (m, 1H), 4.11-4.09 (m, 2H), 3.94-3.92 (m, 1H), 3.74-3.73 (m, 1H), 3.43 (m, 1H), 3.28-3.26 (m, 2H), 2.89-2.88 (m, 1H), 1.49-1.45 (m, 18H).

### Step 11: Synthesis of Compound 4-6 and a Trifluoroacetate of Compound 4-6

Compound 4-5 (0.52 g, 1.28 mmol, 1 eq) was added to DCM (12 mL) and trifluoroacetic acid (6 mL), and the resulting mixture was reacted at 25° C. for 2 hours. The reaction solution was concentrated under reduced pressure to give a trifluoroacetate of compound 4-6. The trifluoroacetate was dissolved in methanol (5 mL) and water (5 mL). Aqueous ammonia (1 mL) was added dropwise to adjust the pH to 10. The mixture was extracted with dichloromethane (20 mL×2). The organic phases were combined, dried over anhydrous sodium sulfate, filtered and concentrated to give compound 4-6. MS m/z: 208.0 [M+1]⁺.

### Step 12: Synthesis of Compound 4-7

The first batch: Compound BB-6 (0.25 g, 719.23 μmol, 1 eq) and compound 4-6 (277.27 mg, 863.08 μmol, 1.2 eq) were added to dioxane (20 mL) under nitrogen. Cesium carbonate (703.02 mg, 2.16 mmol, 3 eq), 4,5-bis(diphenylphosphino)-9,9-dimethylxanthene (62.42 mg, 107.89 μmol, 0.15 eq), and palladium acetate (16.15 mg, 71.92 μmol, 0.1 eq) were added. The resulting mixture was reacted at 110° C. for 2 hours. The second batch: The operation of the first batch was repeated. The reaction solutions of the two batches were combined and then added to water (30 mL). The mixture was extracted with ethyl acetate (30 mL×3). The organic phases were combined, dried over anhydrous sodium sulfate, and filtered. The filtrate was concentrated under reduced pressure to give a crude product. The crude product was purified by column chromatography (EA/PE=0-30%) to give compound 4-7. MS m/z: 474.0 [M+1]⁺.

### Step 13: Synthesis of Compound 4-8

Bis(neopentyl glycolato)diboron (2.09 g, 9.24 mmol) and KOAc (1.21 g, 12.32 mmol) were added to a solution of compound 4-8a (1.3 g, 6.16 mmol) in dioxane (22 mL). The reaction system was purged with nitrogen three times. Pd(dppf)C₂ (676.03 mg, 923.92 μmol) was added, and the resulting mixture was reacted at 100° C. for 2 hr. Water (30 mL) was added, and the mixture was extracted with EA (30 mL*3). The organic phases were combined, washed with saturated brine (10 mL), dried over anhydrous sodium sulfate, and concentrated to give a crude product. The crude product was purified by column chromatography (EA:PE: 0-20%) to give compound 4-8.

### Step 14: Synthesis of Compound 4-9

Compound 4-7 (0.2 g, 422.02 μmol, 1 eq) and compound 4-8 (206.03 mg, 844.04 mol, 2 eq) were added to dioxane (6 mL) and water (1.2 mL) under nitrogen. Potassium phosphate (268.74 mg, 1.27 mmol, 3 eq) and XPhos Pd G2 (33.20 mg, 42.20 μmol, 0.1 eq) were added. The resulting mixture was reacted at 100° C. for 2 hours. The reaction solution was added to water (30 mL), and the mixture was extracted with ethyl acetate (30 mL×3). The organic phases were combined, dried over anhydrous sodium sulfate, and filtered. The filtrate was concentrated under reduced pressure. A mixture of dichloromethane and methanol (1:10, 30 mL) was added to the crude product. The resulting mixture was stirred for 0.5 hours, and then filtered. The filter cake was dried to give compound 4-9. MS m/z: 570.2 [M+1]⁺; ¹H NMR (400 MHz, CDCl₃) δ ppm 10.05 (s, 1H) 9.23 (s, 1H) 8.35 (d, J=5.0 Hz, 1H) 7.11 (d, J=5.0 Hz, 1H) 7.00 (d, J=8.4 Hz, 1H) 6.82 (m, 1H) 6.48 (d, J=8.4 Hz, 1H) 4.88 (s, 2H) 4.29 (m, 1H) 3.97-4.13 (m, 2H) 3.79-3.93 (m, 4H) 3.69 (m, 1H) 3.36 (m, 1H) 3.20-3.29 (m, 1H) 3.15 (m, 1H) 2.77 (m, 1H) 1.54 (s, 9H).

### Step 15: Synthesis of Compound 4 and a Trifluoroacetate of Compound 4

Compound 4-9 (180 mg, 316.01 μmol) was added to DCM (0.5 mL) and trifluoroacetic acid (0.25 mL), and the resulting mixture was reacted at 25° C. for 2 hours. The mixture was concentrated under reduced pressure, and then 20 ml of dichloromethane was added to dissolve the mixture. The resulting mixture was poured into 30 ml of an aqueous solution of sodium bicarbonate, and the mixture was extracted with dichloromethane (30 ml×3). The organic phases were combined and concentrated under reduced pressure. Then, 10 ml of water was added. The resulting mixture was stirred for 0.5 hours and filtered. The filter cake was dried under reduced pressure to give compound 4. MS m/z: 470.1 [M+1]⁺.

Compound 4-9 (5 mg, 8.78 μmol, 1 eq) was added to dichloromethane (0.5 mL) and trifluoroacetic acid (0.25 mL), and the resulting mixture was reacted at 25° C. for 2 hours. The mixture was concentrated under reduced pressure to give a trifluoroacetate of compound 4. MS m/z: 470.1 [M+1]⁺; ¹H NMR (400 MHz, DMSO-d₆) δ ppm 9.85 (s, 1H) 9.45 (s, 1H) 9.15 (s, 1H) 8.35 (d, J=5.0 Hz, 1H) 7.56 (d, J=3.4 Hz, 1H) 7.37 (d, J=5.0 Hz, 1H) 7.32 (d, J=8.6 Hz, 1H) 6.92 (d, J=3.4 Hz, 1H) 6.69 (d, J=8.4 Hz, 1H) 4.71 (s, 2H) 4.36-4.44 (m, 1H) 3.90-4.01 (m, 2H) 3.87 (s, 3H) 3.55-3.63 (m, 2H) 3.17-3.25 (m, 1H) 3.02-3.11 (m, 1H) 1.24 (s, 2H).

### Example 5

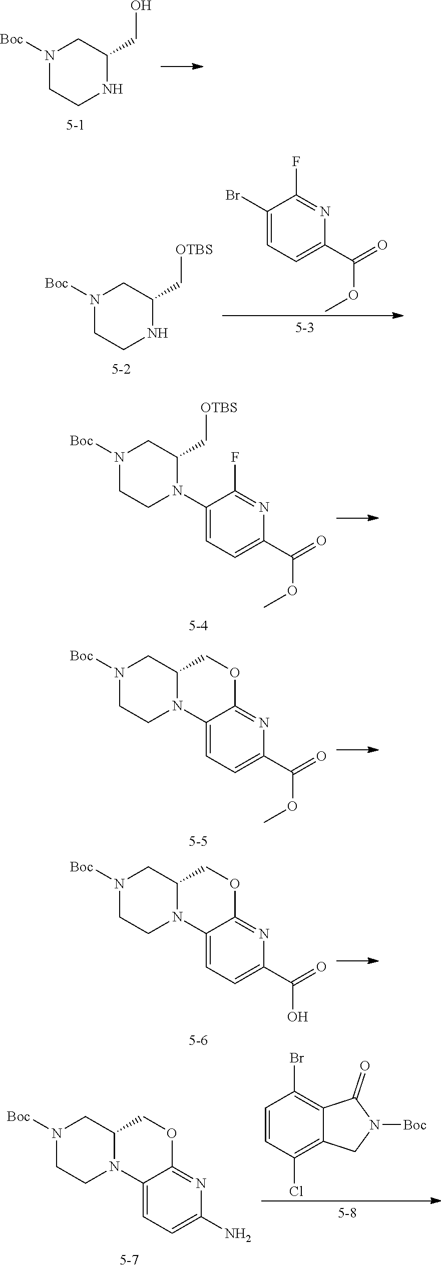

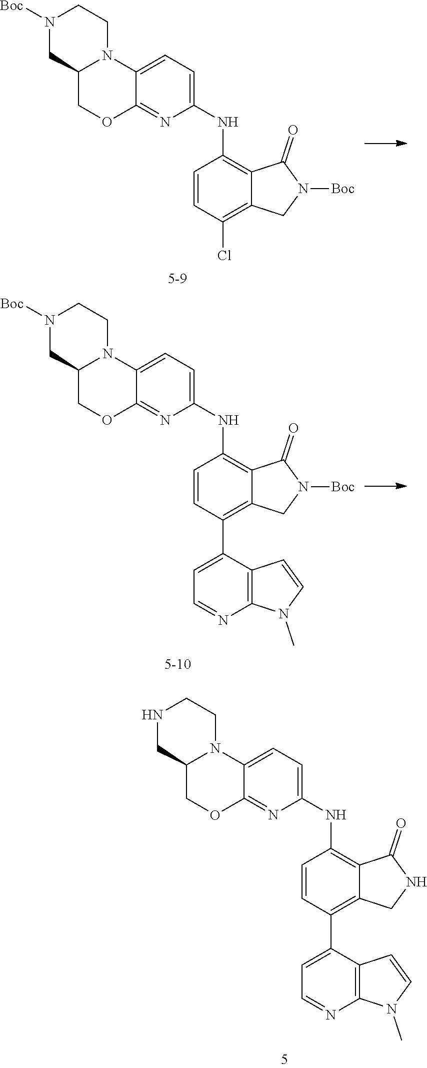 [SMILES: CC(C)(C)OC(=O)N1CCN2c3ccc(Nc4ccc(Cl)c5c4C(=O)N(C(=O)OC(C)(C)C)C5)nc3OC[C@H]2C1 ; Cn1ccc2c(-c3ccc(Nc4ccc5c(n4)OC[C@H]4CN(C(=O)OC(C)(C)C)CCN54)c4c3CN(C(=O)OC(C)(C)C)C4=O)ccnc21 ; Cn1ccc2c(-c3ccc(Nc4ccc5c(n4)OC[C@H]4CNCCN54)c4c3CNC4=O)ccnc21]

### Step 1: Synthesis of Compound 5-2

Compound 5-1 (2 g, 9.25 mmol) was dissolved in DCM (40 mL) under nitrogen, and the mixture was added to a solution of triethylamine (1.87 g, 18.49 mmol, 2.57 ml), TBSCl (2.09 g, 13.87 mmol, 1.70 mL) and 4-dimethylaminopyridine (112.97 mg, 924.74 μmol, 0.1 eq). The reaction solution was stirred at 20° C. for 16 hours. The reaction solution was concentrated under reduced pressure to give a crude product. The crude product was purified by column chromatography (dichloromethane/methanol=5%-10%) to give compound 5-2. ¹H NMR (400 MHz, CD₃OD) δ ppm 3.88-4.12 (m, 2H) 3.63 (d, J=5.6 Hz, 2H) 3.01 (d, J=12.2 Hz, 1H) 2.54-2.94 (m, 4H) 1.48 (s, 9H) 0.95 (s, 9H) 0.12 (s, 6H).

### Step 2: Synthesis of Compound 5-4

Cesium carbonate (197.15 mg, 605.08 μmol), Ruphos (14.12 mg, 30.25 μmol), compound 5-2 (0.1 g, 302.54 μmol), compound 5-3 (70.80 mg, 302.54 μmol), and tris(dibenzylideneacetone)dipalladium(0) (55.41 mg, 60.51 μmol) were added to a toluene (4 mL) solution under nitrogen. The reaction solution was stirred at 100° C. for 24 hours. The reaction solution was filtered under reduced pressure, and the filtrate was concentrated under reduced pressure to give a crude product. The crude product was purified by column chromatography (ethyl acetate/petroleum ether=16%) to give compound 5-4. MS m/z: 484.3 [M+1]⁺;

### Step 3: Synthesis of Compound 5-5

Tetrabutylammonium bromide (1 M, 258.45 μL) and compound 5-4 (50 mg, 103.38 μmol) were added to tetrahydrofuran (4 mL), and the reaction solution was stirred at 25° C. for 16 hours. The reaction solution was concentrated under reduced pressure to give compound 5-5. MS m/z: 350.2 [M+1]⁺.

### Step 4: Synthesis of Compound 5-6

Lithium hydroxide monohydrate (288.26 mg, 6.87 mmol,) and compound 5-5 (0.4 g, 1.14 mmol) were added to a mixed solution of tetrahydrofuran (6 mL), methanol (3 mL), and water (3 mL). The reaction solution was stirred at 25° C. for 0.5 hours. The reaction solution was concentrated under reduced pressure to give compound 5-6. MS m/z: 336.2 [M+1]⁺.

### Step 5: Synthesis of Compound 5-7

Compound 5-6 (0.4 g, 1.19 mmol) and triethylamine (241.39 mg, 2.39 mmol, 332.04 μL) were added to toluene (6 mL). Water (85.95 mg, 4.77 mmol, 85.95 μL) was added to the reaction solution, and then diphenylphosphoryl azide (656.50 mg, 2.39 mmol, 516.93 μL) was added to the reaction solution. The reaction solution was stirred at 90° C. for 2 hours. The reaction solution was concentrated under reduced pressure to give compound 5-7. MS m/z: 307.0 [M+1]⁺.

### Step 6: Synthesis of Compound 5-9

Compound 5-7 (20 mg, 57.70 μmol), di-tert-butyl[2′,4′,6′-tri(propane-2-yl)-[1,1′-biphenyl]-2-yl]phosphine (5.50 mg, 11.54 μmol) and 5-8 (26.52 mg, 86.55 μmol) were added to dioxane (3 mL) under nitrogen, and palladium acetate (1.30 mg, 5.77 μmol) was added to the reaction solution. Then, cesium carbonate (56.40 mg, 173.10 μmol) was added to the reaction solution, and the reaction solution was stirred at 90° C. for 2 hours. The reaction solution was concentrated under reduced pressure to give a crude product. The crude product was separated by column chromatography (methanol/ethyl acetate=10%) to give compound 5-9. MS m/z: 572.1 [M+1]⁺.

### Step 7: Synthesis of Compound 5-10

Compound 5-9 (10.00 mg, 17.48 μmol) and compound 4-8 (6.15 mg, 34.96 μmol) were added to dioxane (2 mL) under nitrogen, and potassium phosphate (11.13 mg, 52.44 μmol) was added to the reaction solution. Then, XPhos Pd G2 (1.38 mg, 1.75 μmol) was added to the reaction solution, and the reaction solution was stirred at 100° C. for 1 hour. The reaction solution was concentrated under reduced pressure to give a crude product. The crude product was separated by column chromatography (ethyl acetate/petroleum ether=50%) to give compound 5-10. MS m/z: 668.1 [M+1]⁺.

### Step 8: Synthesis of Compound 5

Compound 5-10 (6 mg, 8.99 μmol) was added to DCM (2 mL), and trifluoroacetic acid (2.05 mg, 17.97 μmol, 1.33 μL) was added to the reaction solution. The reaction solution was stirred at 20° C. for 1 hour. The reaction solution was concentrated under reduced pressure to give a crude product of raw material. The crude product was separated by column chromatography (ethyl acetate/petroleum ether=50%) to give compound 5. MS m/z: 468.0 [M+1]⁺; ¹H NMR (400 MHz, CD₃OD) δ ppm 8.41 (d, J=8.6 Hz, 1H) 8.25 (d, J=5.4 Hz, 1H) 7.65 (d, J=8.6 Hz, 1H) 7.41 (d, J=3.6 Hz, 1H) 7.23-7.33 (m, 2H) 6.55-6.61 (m, 1H) 6.49 (d, J=3.6 Hz, 1H) 4.39 (s, 2H) 4.12 (m, 1H) 3.93 (d, J=14.8 Hz, 1H) 3.86 (s, 3H) 3.34-3.43 (m, 2H) 2.82-2.93 (m, 2H) 1.93 (d, J=4.2 Hz, 1H) 1.49 (s, 2H).

### Example 6

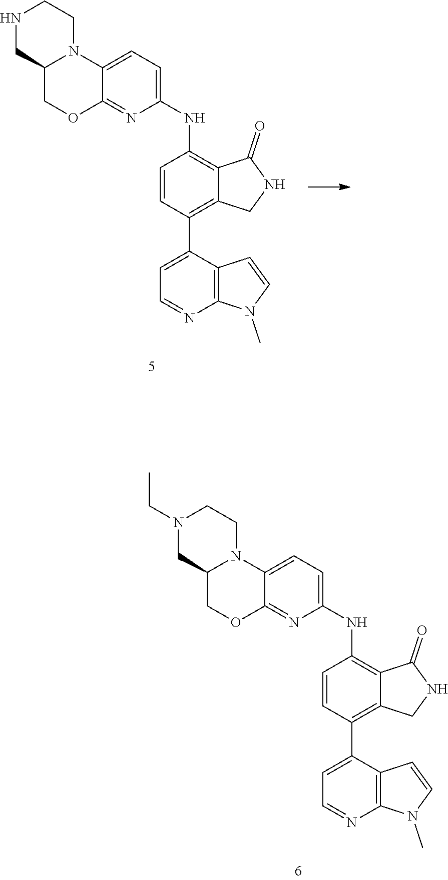 [SMILES: Cn1ccc2c(-c3ccc(Nc4ccc5c(n4)OC[C@H]4CNCCN54)c4c3CNC4=O)ccnc21 ; CCN1CCN2c3ccc(Nc4ccc(-c5ccnc6c5ccn6C)c5c4C(=O)NC5)nc3OC[C@H]2C1]

### Step 1: Synthesis of Compound 6

Compound 5 (8.00 mg, 17.11 μmol) was added to methanol (5 mL). Triethylamine (5.19 mg, 51.33 μmol, 7.15 μL) and an aqueous solution of acetaldehyde (9.42 mg, 85.56 μmol, 12.00 μL, 40% purity) were added. The reaction solution was stirred for 10 minutes. NaBH(OAc)₃ (5.44 mg, 25.67 μmol) was added, and the reaction solution was stirred at 25° C. for 1 hour. Water (5 mL) was added to the reaction solution to quench the reaction. Then, the resulting mixture was extracted with dichloromethane (10 mL×2). The organic phase was dried over anhydrous sodium sulfate, filtered, and concentrated under reduced pressure to give a crude product. The crude product was purified by a preparative thin layer chromatography silica gel plate (ethyl acetate/methanol=1:1, v %=0.5% triethylamine) to give compound 6. MS m/z: 496.2 [M+1]⁺; ¹H NMR (400 MHz, CD₃OD) δ ppm 8.52 (d, J=8.6 Hz, 1H) 8.35 (d, J=5.4 Hz, 1H) 7.75 (d, J=8.6 Hz, 1H) 7.40-7.54 (m, 2H) 7.30 (d, J=5.2 Hz, 1H) 6.70 (d, J=8.2 Hz, 1H) 6.55 (d, J=3.6 Hz, 1H) 4.53 (m, 1H) 4.49 (s, 2H) 4.21-4.30 (m, 1H) 4.12 (d, J=13.2 Hz, 1H) 3.96 (s, 3H) 3.74 (t, J=12.6 Hz, 2H) 3.50 (s, 1H) 3.40-3.44 (m, 2H) 3.11-3.27 (m, 1H) 2.93-3.04 (m, 2H) 1.44 (t, J=7.4 Hz, 3H).

### Example 7

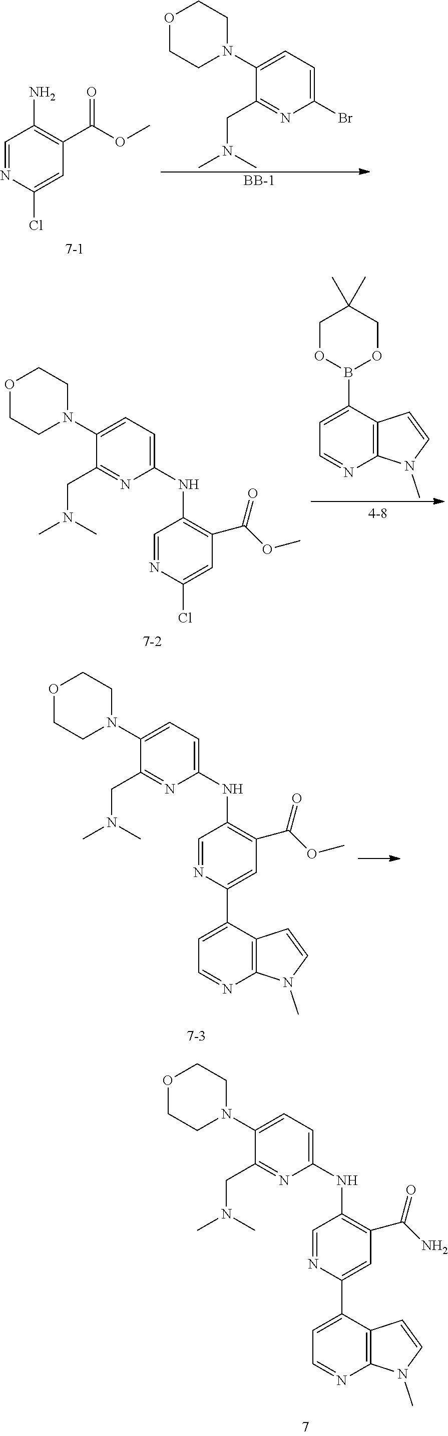

### Step 1: Synthesis of Compound 7-2

Compound 7-1 (200 mg, 1.07 mmol, 1 eq) was dissolved in dioxane (5 mL) under nitrogen. Compound BB-1 (321.76 mg, 1.07 mmol, 1 eq), cesium carbonate (698.45 mg, 2.14 mmol, 2 eq), Xantphos (186.06 mg, 321.55 μmol, 0.3 eq), and Pd₂(dba)₃ (196.30 mg, 214.37 mol, 0.2 eq) were added. The resulting mixture was reacted at 110° C. for 2 hours. The mixture was cooled, and water (10 mL) was added. The mixture was extracted with ethyl acetate (20 mL×2). The organic phases were combined, dried over anhydrous sodium sulfate, filtered, and concentrated to give a crude product. The crude product was purified by column chromatography (methanol/ethyl acetate=0%-33%) to give compound 7-2. MS m/z: 406.1 [M+1]⁺.

### Step 2: Synthesis of Compound 7-3

Compound 7-2 (90 mg, 221.74 μmol, 1 eq) was dissolved in dioxane (2 mL) and water (0.4 mL) under nitrogen. Compound 4-8 (64.95 mg, 266.09 μmol, 1.2 eq), potassium carbonate (76.62 mg, 554.35 μmol, 2.5 eq), and Pd(dppf)Cl₂ (14.45 mg, 22.17 μmol, 0.1 eq) were added. The resulting mixture was reacted at 100° C. for 2 hours. The mixture was cooled. The reaction solution was purified by a preparative thin layer chromatography plate (ethyl acetate/methanol=1:1, v %=0.5% triethylamine) to give compound 7-3. MS m/z: 502.4 [M+1]⁺.

### Step 3: Synthesis of Compound 7

A solution of lithium bis(trimethylsilyl)amide in tetrahydrofuran (1 M, 1.28 mL, 8 eq) was added to a mixture of compound 7-3 (80 mg, 159.50 μmol, 1 eq) and ammonium chloride (51.19 mg, 956.98 μmol, 6 eq) under nitrogen. The resulting mixture was reacted at 20° C. for 0.5 h. Methanol (3 mL) was added to quench the reaction. The solvent was concentrated under reduced pressure to give a crude product. The crude product was purified by prep-HPLC (chromatography column: Waters Xbridge BEH C18 100*30 mm*10 μm; mobile phase: [water (NH₄HCO₃)-acetonitrile]; acetonitrile %: 15%-45%, 8 min) to give compound 7. MS m/z: 487.2 [M+1]⁺; ¹H NMR (400 MHz, DMSO-d₆) δ 10.67 (s, 1H), 10.09 (s, 1H), 8.63 (s, 1H), 8.35 (d, J=5.2 Hz, 1H), 8.28 (s, 1H), 7.97 (s, 1H), 7.66 (d, J=4.8 Hz, 1H), 7.60-7.52 (m, 2H), 7.11 (d, J=3.6 Hz, 1H), 6.95 (d, J=8.8 Hz, 1H), 3.87 (s, 3H), 3.77-3.72 (m, 4H), 3.55 (s, 2H), 3.01-2.91 (m, 4H), 2.30 (s, 6H).

### Example 8

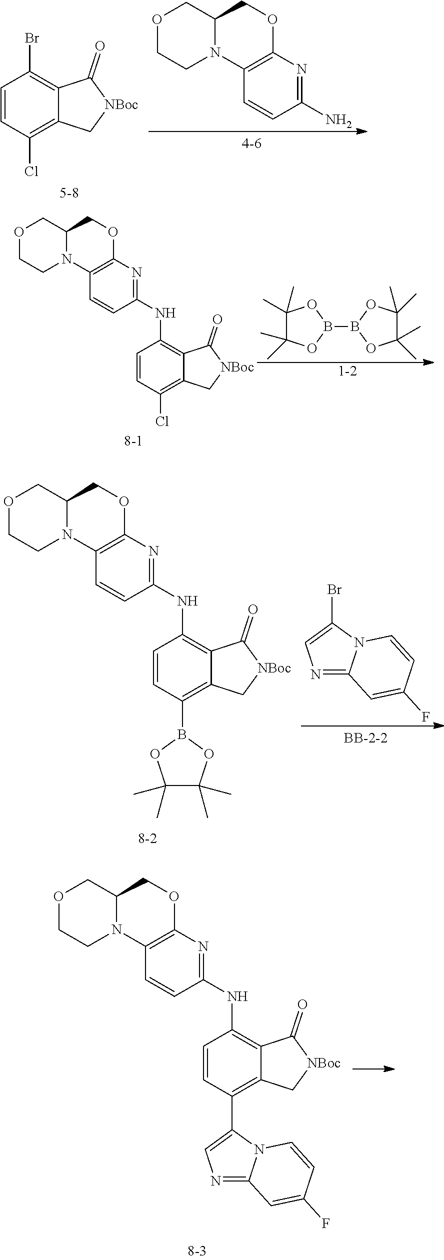

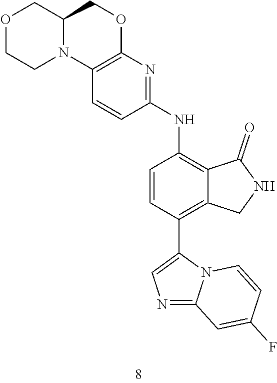 [SMILES: O=C1NCc2c(-c3cnc4cc(F)ccn34)ccc(Nc3ccc4c(n3)OC[C@@H]3COCCN43)c21]

### Step 1: Synthesis of Compound 8-2

Compound 5-8 (300 mg, 865.54 μmol, 1 eq) was dissolved in dioxane (10 mL) under nitrogen. The trifluoroacetate of compound 4-6 (452.10 mg, 1.04 mmol, 2.52 eq), cesium carbonate (1.13 g, 3.46 mmol, 4 eq), Xantphos (75.12 mg, 129.83 μmol, 0.15 eq), and palladium acetate (19.43 mg, 86.55 μmol, 0.1 eq) were added. The resulting mixture was reacted at 110° C. for 2 hours. The mixture was cooled, vacuum filtered, and washed with dichloromethane (20 mL). The filtrate was concentrated under reduced pressure to give a crude product. The crude product was purified by column chromatography (ethyl acetate/petroleum ether=0%-40%) to give compound 8-1. MS m/z: 473.1 [M+1]⁺; ¹H NMR (400 MHz, CDCl₃) δ 9.46 (s, 1H), 8.58 (d, J=8.8 Hz, 1H), 7.42 (d, J=8.8 Hz, 1H), 7.05 (d, J=8.4 Hz, 1H), 6.47 (d, J=8.4 Hz, 1H), 4.67 (s, 2H), 4.36-4.33 (m, 1H), 4.20-4.10 (m, 1H), 4.09-4.02 (m, 1H), 3.94-3.91 (m, 1H), 3.79-3.72 (m, 1H), 3.41 (d, J=11.6 Hz, 1H), 3.36-3.26 (m, 1H), 3.23-3.14 (m, 1H), 2.85-2.79 (m, 1H), 1.63 (s, 9H).

### Step 2: Synthesis of Compound 8-2

Compound 8-1 (211 mg, 446.16 μmol, 1 eq) was dissolved in dioxane (5 mL) under nitrogen. Compound 1-2 (339.89 mg, 1.34 mmol, 3 eq), potassium acetate (131.36 mg, 1.34 mmol, 3 eq), Xphos (42.54 mg, 89.23 μmol, 0.2 eq), and XPhos Pd G2 (35.10 mg, 44.62 mol, 0.1 eq) were added. The resulting mixture was reacted at 110° C. for 2 hours. The mixture was cooled, vacuum filtered, and washed with dichloromethane (20 mL). The filtrate was concentrated under reduced pressure. The resulting crude product was purified by column chromatography (ethyl acetate/petroleum ether=0%-30%) to give compound 8-2. MS m/z: 565.2 [M+1]⁺; ¹H NMR (400 MHz, DMSO-d₆) δ 9.57 (s, 1H), 8.26 (d, J=8.8 Hz, 1H), 7.76 (d, J=8.4 Hz, 1H), 7.29 (d, J=8.4 Hz, 1H), 6.69 (d, J=8.4 Hz, 1H), 4.75 (s, 2H), 4.39-4.35 (m, 1H), 4.07-4.02 (m, 1H), 4.00-3.93 (m, 1H), 3.91-3.89 (m, 1H), 3.64-3.57 (m, 2H), 3.23-3.14 (m, 1H), 3.10-3.00 (m, 1H), 2.67-2.61 (m, 1H), 1.53 (s, 9H), 1.30 (s, 12H).

### Step 3: Synthesis of Compound 8-3

Compound 8-2 (107 mg, 189.57 μmol, 1 eq) was dissolved in dioxane (5 mL) and water (1 mL) under nitrogen. Compound BB-2-2 (48.91 mg, 227.48 μmol, 1.2 eq), potassium phosphate (80.48 mg, 379.14 μmol, 2 eq), and XPhos Pd G2 (14.92 mg, 18.96 μmol, 0.1 eq) were added. The resulting mixture was reacted at 100° C. for 2 hours. Water (10 mL) was added to quench the reaction. The mixture was extracted with ethyl acetate (20 mL×3). The organic phase was washed with saturated brine (10 mL), dried over anhydrous sodium sulfate, vacuum filtered, and concentrated under reduced pressure to give compound 8-3. MS m/z: 573.4 [M+1]⁺.

### Step 4: Synthesis of Compound 8

Compound 8-3 (201 mg, 351.04 μmol, 1 eq) was dissolved in DCM (4 mL) under nitrogen. Trifluoroacetic acid (3.08 g, 27.01 mmol, 2 mL, 76.95 eq) was added, and the resulting mixture was reacted at 20° C. for 2 hours. The solvent was concentrated under reduced pressure to give a crude product. The crude product was purified by prep-HPLC (chromatography column: Phenomenex C18 75*30 mm*3 μm; mobile phase: [water (NH₄HCO₃)-acetonitrile]; acetonitrile %: 15%-50%, 8 min) to give compound 8. MS m/z: 473.1 [M+1]⁺; ¹H NMR (400 MHz, DMSO-d₆) δ 9.77 (s, 1H), 8.75 (s, 1H), 8.48 (d, J=8.4 Hz, 1H), 8.43-8.39 (m, 1H), 7.80 (s, 1H), 7.68 (d, J=8.8 Hz, 1H), 7.53-7.50 (m, 1H), 7.28 (d, J=8.4 Hz, 1H), 6.98-6.94 (m, 1H), 6.59 (d, J=8.4 Hz, 1H), 4.36 (s, 3H), 4.07-3.84 (m, 3H), 3.66-3.53 (m, 2H), 3.23-3.14 (m, 1H), 3.07-2.98 (m, 1H), 2.69-2.64 (m, 1H).

### Example 9

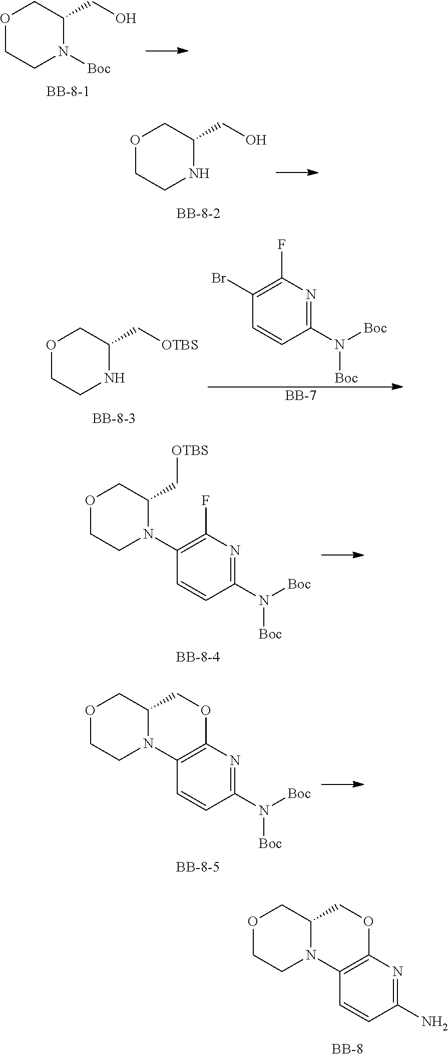

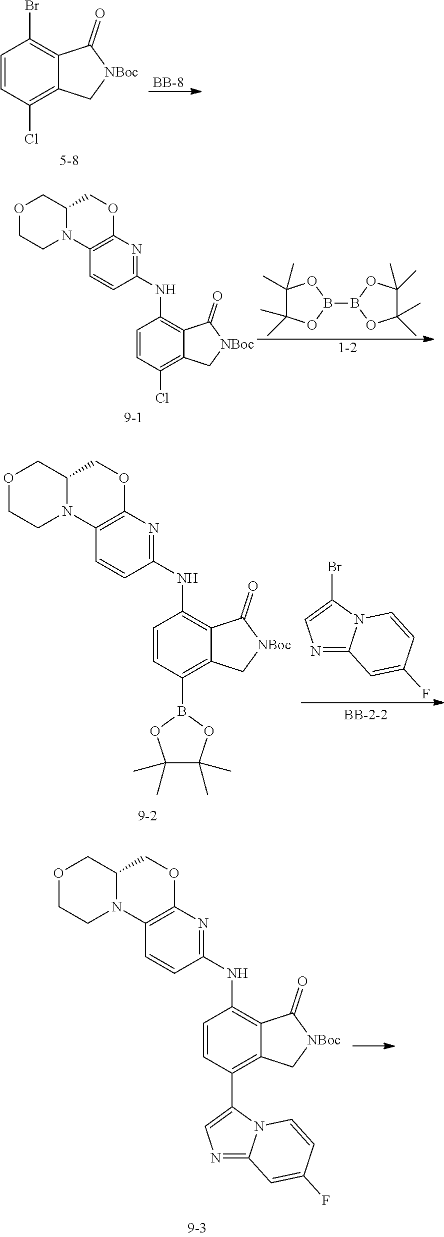

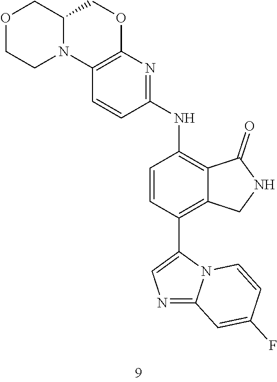 [SMILES: O=C1NCc2c(-c3cnc4cc(F)ccn34)ccc(Nc3ccc4c(n3)OC[C@H]3COCCN43)c21]

### Step 1: Synthesis of a Hydrochloride of Compound BB-8-2

Compound BB-8-1 (5 g, 23.01 mmol, 1 eq) was added to a hydrochloric acid/methanol solution (4 M, 100.00 mL, 17.38 eq) under nitrogen. The resulting mixture was reacted at 20° C. for 2 hours. The solvent was concentrated under reduced pressure to give a hydrochloride of BB-8-2. ¹H NMR (400 MHz, DMSO-d₆) δ 9.50 (s, 1H), 9.27-9.06 (m, 1H), 3.97-3.81 (m, 2H), 3.73-3.47 (m, 4H), 3.28-2.96 (m, 3H).

### Step 2: Synthesis of Compound BB-8-3

The hydrochloride of compound BB-8-2 (2.7 g, 23.05 mmol, 1 eq) was added to DCM (54 mL) under nitrogen. Triethylamine (7.00 g, 69.14 mmol, 9.62 mL, 3 eq), imidazole (1.88 g, 27.66 mmol, 1.2 eq), and TBSCl (4.17 g, 27.66 mmol, 3.39 mL, 1.2 eq) were added. The resulting mixture was reacted at 20° C. for 16 hours. A saturated aqueous solution of ammonium chloride (40 mL) was added to quench the reaction. The mixture was extracted with dichloromethane (50 mL×3). The organic phase was washed with saturated brine (20 mL), dried over anhydrous sodium sulfate, vacuum filtered, and concentrated under reduced pressure. The resulting crude product was purified by column chromatography (methanol/dichloromethane=0-10%) to give compound BB-8-3. ¹H NMR (400 MHz, CDCl₃) δ 3.81-3.77 (m, 2H), 3.60-3.41 (m, 3H), 3.26-3.23 (m, 1H), 3.02-2.80 (m, 3H), 1.96 (s, 1H), 0.90 (s, 9H), 0.06 (s, 6H).

### Step 3: Synthesis of Compound BB-8-4

Compound BB-7 (1.7 g, 4.35 mmol, 1 eq) was dissolved in toluene (34 mL) under nitrogen. Compound BB-8-3 (1.01 g, 4.35 mmol, 1 eq), cesium carbonate (2.83 g, 8.69 mmol, 2 eq), Ruphos (405.53 mg, 869.05 μmol, 0.2 eq), and Pd₂(dba)₃ (596.85 mg, 651.79 μmol, 0.15 eq) were added. The resulting mixture was reacted at 100° C. for 16 hours. The mixture was cooled and filtered. The residue was washed with dichloromethane (30 mL). The filtrate was concentrated under reduced pressure. The resulting crude product was purified by column chromatography (ethyl acetate/petroleum ether=0%-10%) to give compound BB-8-4. MS m/z: 542.3 [M+1]⁺; ¹H NMR (400 MHz, DMSO-d₆) δ 7.57-7.52 (m, 1H), 7.20 (d, J=8.0 Hz, 1H), 3.93-3.77 (m, 3H), 3.76-3.69 (m, 1H), 3.66-3.48 (m, 3H), 3.29 (s, 1H), 2.94 (d, J=12.4 Hz, 1H), 1.38 (s, 18H), 0.77 (s, 9H), −0.07-0.10 (d, J=9.6 Hz, 6H).

### Step 4: Synthesis of Compound BB-8-5

Compound BB-8-4 (484 mg, 893.44 μmol, 1 eq) was dissolved in tetrahydrofuran (10 mL) under nitrogen. A solution of tetrabutylammonium fluoride in tetrahydrofuran (1 M, 3.69 mL, 2.5 eq) was added. The resulting mixture was reacted at 50° C. for 16 hours. The mixture was cooled, and the reaction solution was concentrated under reduced pressure. The resulting crude product was purified by column chromatography (ethyl acetate/petroleum ether=0%-30%) to give compound BB-8-5. MS m/z: 408.1 [M+1]⁺; ¹H NMR (400 MHz, DMSO-d₆) δ 7.24 (d, J=8.0 Hz, 1H), 6.79 (d, J=8.0 Hz, 1H), 4.37-4.34 (m, 1H), 4.07-3.95 (m, 2H), 3.89 (d, J=8.4 Hz, 1H), 3.64-3.52 (m, 2H), 3.21-3.07 (m, 2H), 2.75-2.65 (m, 1H), 1.39 (s, 18H).

### Step 5: Synthesis of a Trifluoroacetate of Compound BB-8

Compound BB-8-5 (269 mg, 660.19 μmol, 1 eq) was dissolved in DCM (5 mL) under nitrogen. Trifluoroacetic acid (3.95 g, 34.67 mmol, 2.57 mL, 52.51 eq) was added, and the resulting mixture was reacted at 25° C. for 2 hours. The reaction solution was concentrated under reduced pressure to give a trifluoroacetate of compound BB-8. MS m/z: 208.1 [M+1]⁺.

### Step 6: Synthesis of Compound 9-1

Compound 5-8 (165 mg, 476.05 μmol, 1 eq) was dissolved in dioxane (5 mL) under nitrogen. The trifluoroacetate of compound BB-8 (248.65 mg, 571.26 μmol, 1.2 eq), cesium carbonate (620.42 mg, 1.90 mmol, 4 eq), Xantphos (41.32 mg, 71.41 μmol, 0.15 eq), and palladium acetate (10.69 mg, 47.60 μmol, 0.1 eq) were added. The resulting mixture was reacted at 110° C. for 2 hours. The mixture was cooled and vacuum filtered. The residue was washed with dichloromethane (20 mL). The filtrate was concentrated under reduced pressure. The resulting crude product was purified by column chromatography (ethyl acetate/petroleum ether=0%-35%) to give compound 9-1. MS m/z: 473.1 [M+1]⁺; ¹H NMR (400 MHz, DMSO-d₆) δ 9.28 (s, 1H), 8.40 (d, J=8.8 Hz, 1H), 7.59 (d, J=8.8 Hz, 1H), 7.28 (d, J=8.4 Hz, 1H), 6.67 (d, J=8.4 Hz, 1H), 4.69 (s, 2H), 4.37-4.34 (m, 1H), 4.04-3.86 (m, 3H), 3.64-3.53 (m, 2H), 3.23-3.13 (m, 1H), 3.09-2.98 (m, 1H), 2.71-2.63 (m, 1H), 1.53 (s, 9H).

### Step 7: Synthesis of Compound 9-2

Compound 9-1 (170 mg, 359.47 μmol, 1 eq) was dissolved in dioxane (4 mL) under nitrogen. Compound 1-2 (273.85 mg, 1.08 mmol, 3 eq), potassium acetate (105.83 mg, 1.08 mmol, 3 eq), XPhos (34.27 mg, 71.89 μmol, 0.2 eq), and XPhos Pd G2 (28.28 mg, 35.95 mol, 0.1 eq) were added. The resulting mixture was reacted at 110° C. for 2 hours. The mixture was cooled and vacuum filtered. The residue was washed with dichloromethane (20 mL). The filtrate was concentrated under reduced pressure. The resulting crude product was purified by column chromatography (ethyl acetate/petroleum ether=0%-35%) to give compound 9-2. MS m/z: 565.2 [M+1]⁺; ¹H NMR (400 MHz, CDCl₃) δ 9.76 (s, 1H), 8.42 (d, J=8.8 Hz, 1H), 7.89 (d, J=8.4 Hz, 1H), 7.05 (d, J=8.4 Hz, 1H), 6.52 (d, J=8.4 Hz, 1H), 4.86 (s, 2H), 4.36-4.33 (m, 1H), 4.19-4.11 (m, 1H), 4.09-4.05 (m, 1H), 3.94-3.90 (m, 1H), 3.79-3.72 (m, 1H), 3.41 (d, J=12.0 Hz, 1H), 3.34-3.26 (m, 1H), 3.23-3.14 (m, 1H), 2.90-2.76 (m, 1H), 1.64 (s, 9H), 1.33 (s, 12H).

### Step 8: Synthesis of Compound 9-3

Compound 9-2 (134 mg, 237.41 μmol, 1 eq) was dissolved in dioxane (5 mL) and water (1 mL) under nitrogen. Compound BB-2-2 (61.26 mg, 284.89 μmol, 1.2 eq), potassium phosphate (100.79 mg, 474.81 μmol, 2 eq), and XPhos Pd G2 (18.68 mg, 23.74 μmol, 0.1 eq) were added. The resulting mixture was reacted at 100° C. for 2 hours. The mixture was cooled and vacuum filtered. The residue was washed with dichloromethane (10 mL). The filtrate was concentrated under reduced pressure. The resulting crude product was purified by column chromatography (ethyl acetate/petroleum ether=0%-70%) to give compound 9-3. MS m/z: 573.4 [M+1]⁺.

### Step 9: Synthesis of Compound 9 and a Trifluoroacetate of Compound 9

Compound 9-3 (125 mg, 218.31 μmol, 1 eq) was dissolved in DCM (4 mL) under nitrogen. Trifluoroacetic acid (3.08 g, 27.01 mmol, 2 mL, 76.95 eq) was added, and the resulting mixture was reacted at 20° C. for 2 hours. The solvent was concentrated under reduced pressure to give a crude product. The crude product was purified by prep-HPLC (chromatography column: Phenomenex C18 75*30 mm*3 μm; mobile phase: [water (NH₄HCO₃)-acetonitrile]; acetonitrile %: 20%-50%, 8 min) to give compound 9. MS m/z: 473.1 [M+1]⁺; ¹H NMR (400 MHz, DMSO-d₆) δ 9.77 (s, 1H), 8.75 (s, 1H), 8.48 (d, J=8.4 Hz, 1H), 8.43-8.39 (m, 1H), 7.80 (s, 1H), 7.67 (d, J=8.8 Hz, 1H), 7.53-7.50 (m, 1H), 7.28 (d, J=8.4 Hz, 1H), 6.98-6.94 (m, 1H), 6.59 (d, J=8.4 Hz, 1H), 4.40-4.31 (m, 3H), 4.07-3.86 (m, 3H), 3.66-3.52 (m, 2H), 3.25-3.14 (m, 1H), 3.09-2.99 (m, 1H), 2.68-2.64 (m, 1H).

Compound 9-3 (0.4 g, 698.59 μmol) was dissolved in DCM (10 mL) under nitrogen, and trifluoroacetic acid (5 mL) was added. The resulting mixture was reacted at 25° C. for 2 hours. The solvent was concentrated under reduced pressure to give a crude product. The crude product was purified by prep-HPLC (chromatography column: Phenomenex Luna 80*30 mm*3 μm; mobile phase: [water (TFA)-acetonitrile]; B %: 10%-35%, 8 min) to give a trifluoroacetate of compound 9. MS m/z: 473.1 [M+1]⁺; 1H NMR (400 MHz, DMSO-d₆) δ ppm 9.82 (s, 1H) 8.81 (s, 1H) 8.66 (dd, J=7.44, 5.32 Hz, 1H) 8.52 (d, J=8.64 Hz, 1H) 8.25 (s, 1H) 7.92 (dd, J=8.76, 2.24 Hz, 1H) 7.72 (d, J=8.62 Hz, 1H) 7.39-7.38 (m, 1H) 7.30 (d, J=8.50 Hz, 1H) 6.62 (d, J=8.38 Hz, 1H) 4.39-4.36 (m, 3H) 4.07-4.04 (m, 2H) 3.91 (dd, J=10.94, 2.80 Hz, 1H) 3.61-3.56 (m, 2H) 3.20-3.17 (m, 1H) 3.04 (s, 1H) 2.66-2.65 (m, 1H).

### Example 10

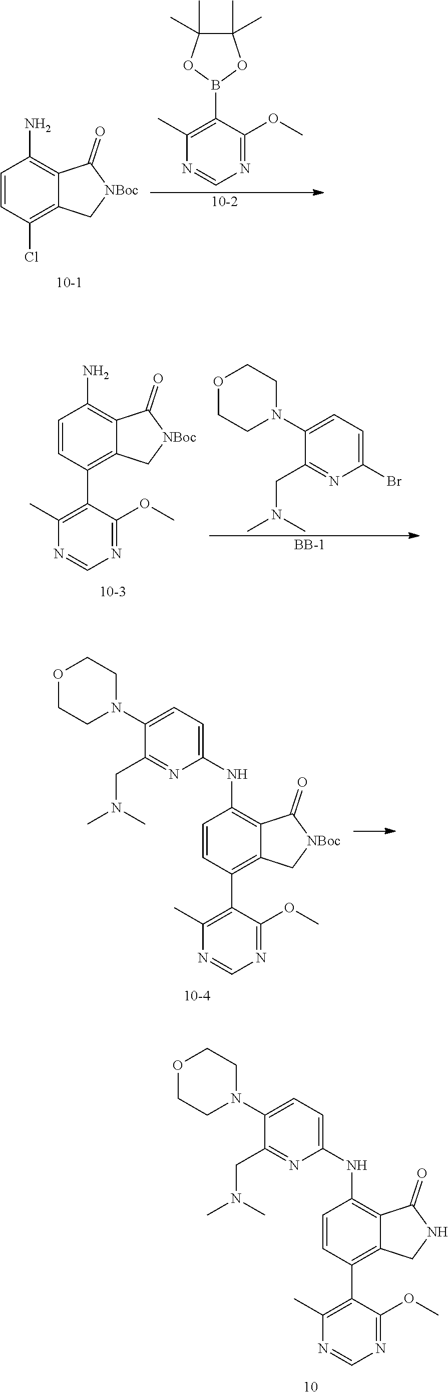

### Step 1: Synthesis of Compound 10-3

Compound 10-1 (0.7 g, 2.48 mmol, 1 eq) was dissolved in dioxane (6 mL) and water (1.2 mL) under nitrogen. 10-2 (1.24 g, 4.95 mmol, 2 eq), XPhos Pd G2 (194.81 mg, 247.59 mol, 0.1 eq), and potassium phosphate (1.05 g, 4.95 mmol, 2 eq) were added. The resulting mixture was reacted at 100° C. for 1 hour. The mixture was cooled, and water (5 mL) was added. The mixture was extracted with ethyl acetate (10 mL). The organic phase was dried over anhydrous sodium sulfate, filtered and concentrated to give a crude product. The crude product was purified by column chromatography (ethyl acetate/petroleum ether=50%-100%) to give compound 10-3. MS m/z: 371.2 [M+1]⁺; ¹H NMR (400 MHz, DMSO-d₆) δ 8.63 (s, 1H), 7.19-7.07 (m, 1H), 6.72-6.62 (m, 1H), 6.49 (s, 2H), 3.81 (s, 3H), 3.29 (s, 2H), 2.15 (s, 3H), 1.43 (s, 9H).

### Step 2: Synthesis of Compound 10-4

Compound 10-3 (0.2 g, 539.95 μmol, 1 eq) was dissolved in dioxane (10 mL) under nitrogen. BB-1 (194.51 mg, 647.95 μmol, 1.2 eq), 4,5-bis(diphenylphosphino)-9,9-dimethylxanthene (46.86 mg, 80.99 μmol, 0.15 eq), palladium acetate (12.12 mg, 54.00 μmol, 0.1 eq), and cesium carbonate (527.78 mg, 1.62 mmol, 3 eq) were added. The resulting mixture was reacted at 100° C. for 1 hour. The mixture was cooled, and water (50 mL) was added. The mixture was extracted with ethyl acetate (50 mL×3). The organic phase was dried over anhydrous sodium sulfate, filtered, and concentrated to give compound 10-4. MS m/z: 590.3 [M+1]⁺.

### Step 3: Synthesis of Compound 10

Compound 10-4 (0.4 g, 678.33 μmol, 1 eq) was dissolved in a solution of hydrochloric acid/methanol (40 mL) under nitrogen, and the resulting mixture was reacted at 25° C. for 2 hours. The reaction system was concentrated under reduced pressure to give a crude product. The crude product was purified by prep-HPLC (chromatography column: Waters Xbridge Prep OBD C18 150*40 mm*10 μm; mobile phase: [water (NH₄HCO₃)-acetonitrile]; acetonitrile %: 10%-60%, 8 min) to give compound 10. MS m/z: 490.4 [M+1]⁺; ¹H NMR (400 MHz, CDCl₃) δ ppm 9.76 (s, 1H) 8.86 (d, J=8.6 Hz, 1H) 8.72 (s, 1H) 7.43 (d, J=8.6 Hz, 1H) 7.31 (s, 1H) 6.84 (d, J=8.6 Hz, 1H) 6.07 (s, 1H) 4.24-4.20 (m, 1H) 4.07-4.03 (m, 1H) 3.95 (s, 3H) 3.89-3.88 (m, 4H) 3.87-3.73 (m, 2H) 3.30-3.01 (m, 4H) 2.98 (s, 6H) 2.44-2.34 (m, 3H).

### Example 11 and Example 12

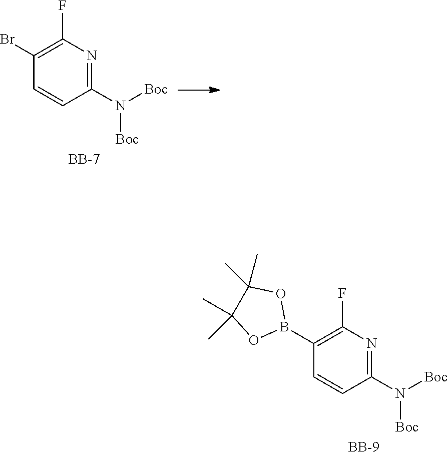 [SMILES: CC(C)(C)OC(=O)N(C(=O)OC(C)(C)C)c1ccc(Br)c(F)n1 ; "BB-7" ; CC(C)(C)OC(=O)N(C(=O)OC(C)(C)C)c1ccc(B2OC(C)(C)C(C)(C)O2)c(F)n1 ; "BB-9"]

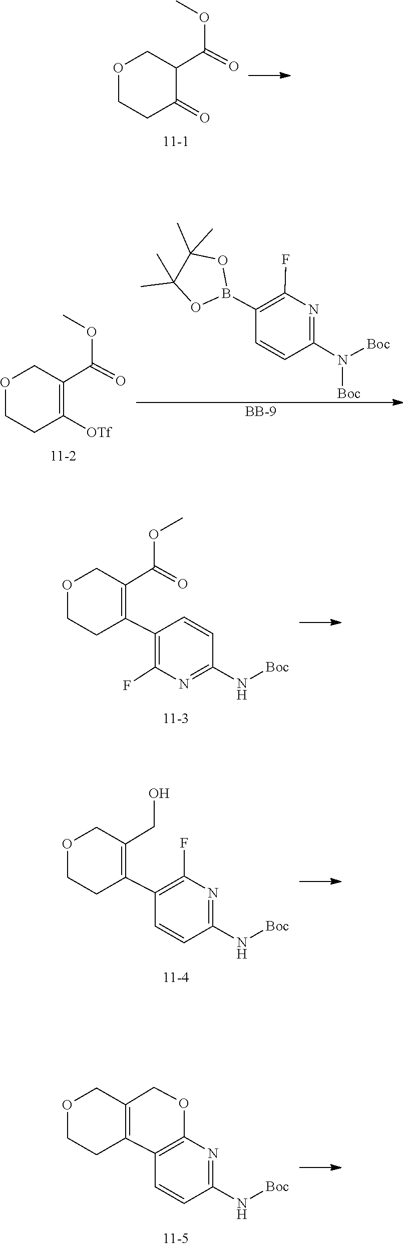

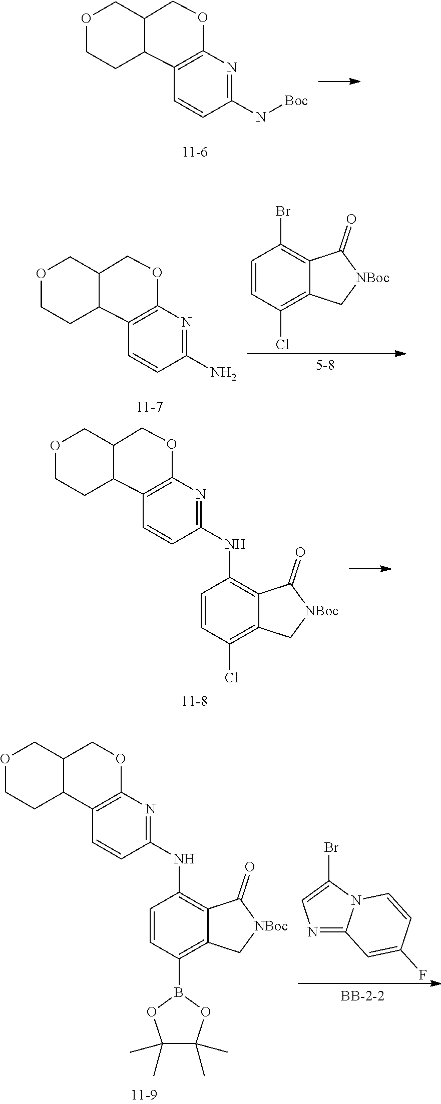 [SMILES: CC(C)(C)OC(=O)Nc1ccc2c(n1)OCC1COCCC21 ; Nc1ccc2c(n1)OCC1COCCC21 ; CC(C)(C)OC(=O)N1Cc2c(Cl)ccc(Br)c2C1=O ; CC(C)(C)OC(=O)N1Cc2c(Cl)ccc(Nc3ccc4c(n3)OCC3COCCC43)c2C1=O ; CC(C)(C)OC(=O)N1Cc2c(B3OC(C)(C)C(C)(C)O3)ccc(Nc3ccc4c(n3)OCC3COCCC43)c2C1=O ; Fc1ccn2c(Br)cnc2c1]

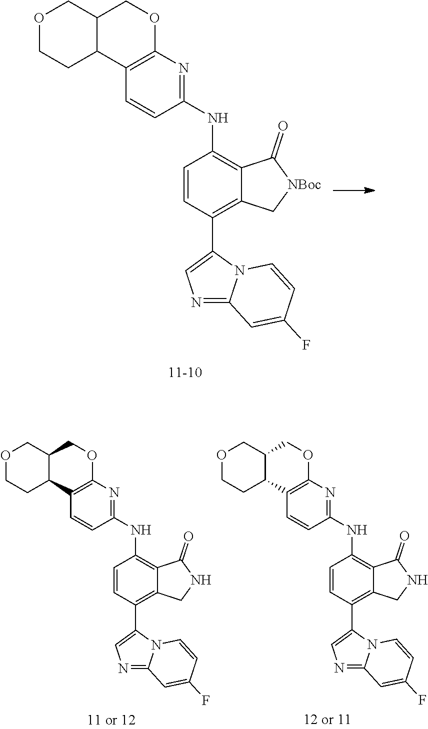 [SMILES: CC(C)(C)OC(=O)N1Cc2c(-c3cnc4cc(F)ccn34)ccc(Nc3ccc4c(n3)OCC3COCCC43)c2C1=O ; O=C1NCc2c(-c3cnc4cc(F)ccn34)ccc(Nc3ccc4c(n3)OC[C@@H]3COCC[C@H]43)c21 ; O=C1NCc2c(-c3cnc4cc(F)ccn34)ccc(Nc3ccc4c(n3)OC[C@H]3COCC[C@@H]43)c21]

### Step 1: Synthesis of Compound BB-9

Compound BB-7 (8 g, 20.45 mmol, 1 eq) was dissolved in dioxane (120 mL). Bis(pinacolato)diboron (7.79 g, 30.67 mmol, 1.5 eq), potassium acetate (4.01 g, 40.90 mmol, 2 eq), and Pd(dppfCl₂·CH₂Cl₂ (1.67 g, 2.04 mmol, 0.1 eq) were added. The resulting mixture was reacted at 120° C. for 12 hours. The mixture was cooled and filtered. The filtrate was poured into water (200 mL). The resulting mixture was extracted with ethyl acetate (200 mL×3). The organic phases were combined, dried over anhydrous sodium sulfate, filtered, and concentrated to give a crude product. The crude product was purified by column chromatography (petroleum ether:ethyl acetate=0%-4%) to give compound BB-9. MS m/z: 200.8 [M-238]⁺.

### Step 2: Synthesis of Compound 11-2

Compound 11-1 (3 g, 18.97 mmol, 1 eq) was added to tetrahydrofuran (45 mL), and the mixture was cooled to 0° C. Then, sodium hydride (1.14 g, 28.45 mmol, 60% purity, 1.5 eq) was added, and the resulting mixture was stirred for 0.5 hours. Then, N-phenyl-bis(trifluoromethanesulfonimide) (8.13 g, 22.76 mmol, 1.2 eq) was added. The resulting mixture was warmed to 25° C. and reacted for 12 hours. The reaction solution was slowly added to a saturated aqueous solution of ammonium chloride (100 mL). The resulting mixture was extracted with ethyl acetate (100 mL×3). The organic phases were combined, dried over anhydrous sodium sulfate, and filtered. The filtrate was concentrated under reduced pressure. The resulting crude product was purified by column chromatography (ethyl acetate/petroleum ether=0%-4%) to give compound 11-2. ¹H NMR (400 MHz, CDCl₃) δ ppm 4.38 (t, J=2.6 Hz, 2H) 3.82 (t, J=5.6 z, 2H) 3.75 (s, 3H) 2.49-2.45 (m, 2H).

### Step 3: Synthesis of Compound 11-3

Compound 11-2 (2.5 g, 8.61 mmol, 1 eq) and BB-9 (5.85 g, 13.35 mmol, 1.55 eq) were added to dioxane (50 mL) and water (10 mL). Potassium phosphate (5.49 g, 25.84 mmol, 3 eq) and XPhos Pd G2 (677.78 mg, 861.44 μmol, 0.1 eq) were added, and the resulting mixture was reacted at 100° C. for 12 hours. The reaction solution was added to water (100 mL). The resulting mixture was extracted with ethyl acetate (50 mL×3). The organic phases were combined, dried over anhydrous sodium sulfate, and filtered. The filtrate was concentrated under reduced pressure. The resulting crude product was purified by column chromatography (ethyl acetate/petroleum ether=0%-5%) to give compound 11-3. MS m/z: 353.1 [M+1]⁺; ¹H NMR (400 MHz, CDCl₃) δ ppm 7.72 (d, J=8.0 Hz, 1H) 7.45 (t, J=8.8 Hz, 1H) 7.01 (s, 1H) 4.39 (s, 2H) 3.82 (t, J=5.4 Hz, 2H) 3.50 (s, 3H) 2.40 (s, 2H) 1.45 (s, 9H).

### Step 4: Synthesis of Compound 11-4

Compound 11-3 (1.2 g, 3.41 mmol, 1 eq) was added to tetrahydrofuran (24 mL), and the resulting mixture was cooled to 0° C. Then, lithium aluminium hydride (323.15 mg, 8.51 mmol, 2.5 eq) was added, and the resulting mixture was reacted for 2 hours. Water was added dropwise to the reaction solution until the reaction was completely quenched. Anhydrous sodium sulfate was added to dry the mixture, and the mixture was filtered. The filtrate was concentrated under reduced pressure to give compound 11-4. MS m/z: 325.0 [M+1]⁺; ¹H NMR (400 MHz, CDCl₃) δ ppm 7.74-7.72 (m 1H) 7.53-7.48 (m 1H) 7.04 (s, 1H) 4.30 (t, J=2.2 Hz, 2H) 3.90-3.88 (m, 2H) 3.84-3.82 (m, 2H) 2.30 (s, 2H) 1.45 (s, 9H).

### Step 5: Synthesis of Compound 11-5

Compound 11-4 (0.9 g, 2.77 mmol, 1 eq) was added to tetrahydrofuran (20 mL), and the mixture was cooled to 0° C. Sodium hydride (221.96 mg, 5.55 mmol, 60% purity, 2 eq) was added, and the resulting mixture was stirred for 0.5 hours. The mixture was heated to 50° C. and reacted for 2 hours. After the reaction, the reaction solution was slowly added to a saturated aqueous solution of ammonium chloride (50 mL). The resulting mixture was extracted with ethyl acetate (50 mL×3). The organic phases were combined, dried over anhydrous sodium sulfate, and filtered. The filtrate was concentrated under reduced pressure to give compound 11-5. MS m/z: 305.1 [M+1]⁺.

### Step 6: Synthesis of Compound 11-6

Palladium on carbon (0.5 g, 10% purity) was added to a dry hydrogenation bottle. Methanol (30 mL) and compound 11-6 (0.8 g, 2.63 mmol, 1 eq) were added. The resulting mixture was reacted at 30° C. under hydrogen (15 psi) for 2 hours. The resulting mixture was filtered, and the filtrate was concentrated under reduced pressure. The resulting crude product was purified by column chromatography (ethyl acetate/petroleum ether=0%-10%) to give compound 11-6. MS m/z: 307.1 [M+1]⁺; ¹H NMR (400 MHz, CDCl₃) δ ppm 7.43-7.42 (m, 1H) 7.35-7.33 (m, 1H) 6.94 (s, 1H) 4.45-4.39 (m, 1H) 4.24-4.21 (m, 1H) 3.77-3.74 (m, 3H) 3.50-3.47 (m, 1H) 2.93 (m, 1H) 2.15-2.13 (m, 1H) 1.88-1.79 (m, 2H) 1.45 (s, 9H).

### Step 7: Synthesis of a Hydrochloride of Compound 11-7

Compound 11-6 (0.78 g, 2.55 mmol, 1 eq) was added to hydrochloric acid/methanol (4M, 2 mL), and the resulting mixture was reacted at 25° C. for 12 hours. The reaction solution was concentrated under reduced pressure to give a hydrochloride of compound 11-7. MS m/z: 207.0 [M+1]⁺.

### Step 8: Synthesis of Compound 11-8

Compound 5-8 (0.45 g, 1.30 mmol, 1 eq) and the hydrochloride of compound 11-7 (378.12 mg, 1.56 mmol, 1.20 eq) were added to dioxane (20 mL). Then, cesium carbonate (1.69 g, 5.19 mmol, 4 eq), Xantphos (112.68 mg, 194.75 μmol, 0.15 eq), and Pd₂(dba)₃ (118.89 mg, 129.83 μmol, 0.1 eq) were added. The resulting mixture was reacted at 100° C. for 12 hours. The reaction solution was added to water (30 mL), and the resulting mixture was extracted with ethyl acetate (30 mL×3). The organic phases were combined, dried over anhydrous sodium sulfate, filtered and concentrated. The resulting crude product was purified by column chromatography (ethyl acetate/petroleum ether=0%-25%) to give compound 11-8. MS m/z: 472.1 [M+1]⁺; ¹H NMR (400 MHz, DMSO-d₆) δ ppm 9.48 (s, 1H) 8.64 (d, J=9.0 Hz, 1H) 7.65 (d, J=8.8 Hz, 1H) 7.56 (d, J=8.2 Hz, 1H) 6.65 (d, J=8.0 Hz, 1H) 4.71 (s, 2H) 4.37-4.28 (m, 2H) 3.75-3.68 (m, 3H) 3.47-3.46 (m, 1H) 3.03-3.00 (m, 1H) 2.14-2.10 (m, 1H) 1.85-1.82 (m, 2H) 1.54 (s, 9H).

### Step 9: Synthesis of Compound 11-9

Compound 11-8 (0.25 g, 529.74 μmol, 1 eq) and bis(pinacolato)diboron (403.56 mg, 1.59 mmol, 3 eq) were added to dioxane (10 mL). Potassium acetate (155.97 mg, 1.59 mmol, 3 eq), XPhos Pd G2 (41.68 mg, 52.97 μmol, 0.1 eq), and 2-di-tert-butylphosphino-2′,4′,6′-triisopropylbiphenyl (50.51 mg, 105.95 μmol, 0.2 eq) were added. The resulting mixture was reacted at 110° C. for 3 hours. The reaction solution was added to water (40 mL), and the resulting mixture was extracted with ethyl acetate (40 mL×3). The organic phases were combined, dried over anhydrous sodium sulfate, filtered, and concentrated. The resulting crude product was purified by column chromatography (ethyl acetate/petroleum ether=0%-25%) to give compound 11-9. MS m/z: 564.3 [M+1]⁺.

### Step 10: Synthesis of Compound 11-10

Compound 11-9 (275.14 mg, 488.32 μmol, 1.5 eq) and compound BB-2-2 (70 mg, 325.55 μmol, 1 eq) were added to dioxane (10 mL) and water (2 mL). XPhos Pd G2 (25.61 mg, 32.55 μmol, 0.1 eq) and potassium phosphate (207.31 mg, 976.64 μmol, 3 eq) were added. The resulting mixture was reacted at 105° C. for 4 hours. The reaction solution was added to water (40 mL), and the resulting mixture was extracted with ethyl acetate (40 mL×3). The organic phases were combined, dried over anhydrous sodium sulfate, filtered and concentrated. The resulting crude product was purified by column chromatography (ethyl acetate/petroleum ether=0%-50%) to give compound 11-10. MS m/z: 572.2 [M+1]⁺.

### Step 11: Synthesis of a Trifluoroacetate of Compound 11 and a Trifluoroacetate of Compound 12

Compound 11-10 (0.15 g, 262.42 μmol, 1 eq) was added to DCM (10 mL) and trifluoroacetic acid (5 mL). The resulting mixture was reacted at 25° C. for 2 hours. The reaction solution was concentrated under reduced pressure. The resulting product was separated by SFC (chromatography column: DAICEL CHIRALPAK AD (250 mm*30 mm, 10 m); mobile phase: [0.1% NH₃H₂O IPA]; B %: 58%-58%, 16 min). The separated products were concentrated, and 5 drops of trifluoroacetic acid were added, respectively, to give a trifluoroacetate of compound 11 and a trifluoroacetate of compound 12.

The trifluoroacetate of compound 11: MS m/z: 472.1 [M+1]⁺; ¹H NMR (400 MHz, DMSO-d₆) δ ppm 10.04 (s, 1H) 8.87 (s, 1H) 8.75 (d, J=8.6 Hz, 1H) 8.69 (m, 1H) 8.30 (s, 1H) 7.95 (m, 1H) 7.77 (d, J=8.6 Hz, 1H) 7.56 (d, J=7.8 Hz, 1H) 7.43-7.41 (m, 1H) 6.59 (d, J=8.0 Hz, 1H) 4.40-4.38 (m, 3H) 4.35-4.30 (m, 1H) 3.74-3.68 (m, 3H) 3.46 (s, 1H) 3.02-3.00 (m, 1H) 2.13-2.11 (m, 1H) 1.84-1.82 (m, 2H). ee %=100%, retention time: 1.196 min.

The trifluoroacetate of compound 12: MS m/z: 472.1 [M+1]⁺; ¹H NMR (400 MHz, DMSO-d₆) δ ppm 10.06 (s, 1H) 8.88 (s, 1H) 8.76 (d, J=8.6 Hz, 1H) 8.72-8.69 (m, 1H) 8.31 (s, 1H) 7.97 (m, 1H) 7.78 (d, J=8.8 Hz, 1H) 7.58 (d, J=7.8 Hz, 1H) 7.46-7.44 (m, 1H) 6.60 (d, J=7.8 Hz, 1H) 4.41-4.39 (m, 3H) 4.37-4.30 (m, 1H) 3.76-3.70 (m, 3H) 3.48 (s, 1H) 3.03-3.02 (m, 1H) 2.13-2.12 (m, 1H) 1.87-1.84 (m, 2H). ee %=98.46%, retention time: 1.586 min.

The analysis method of ee % detection: chromatography column: Chiralpak AD-3, 50×4.6 mm I.D., 3 μm; mobile phase: A: carbon dioxide B: isopropanol (0.1% IPAm, v/v).

### Example 13 and Example 14

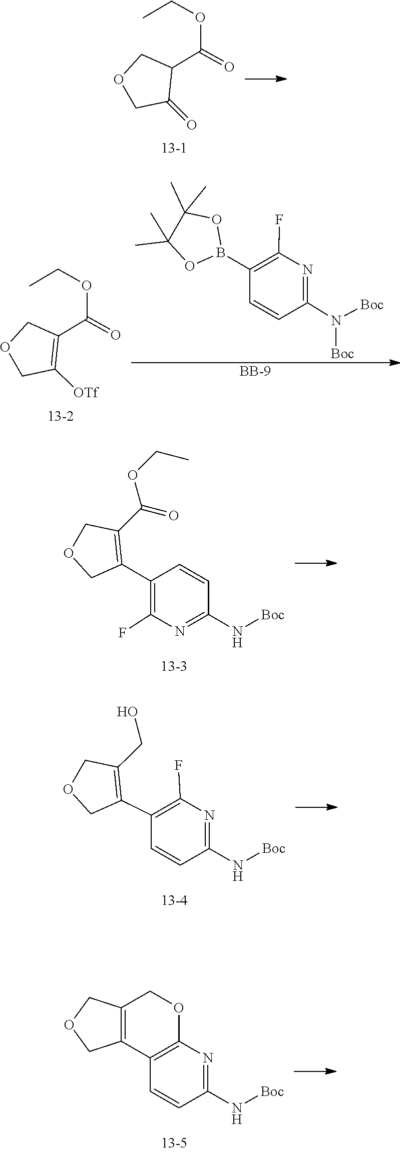

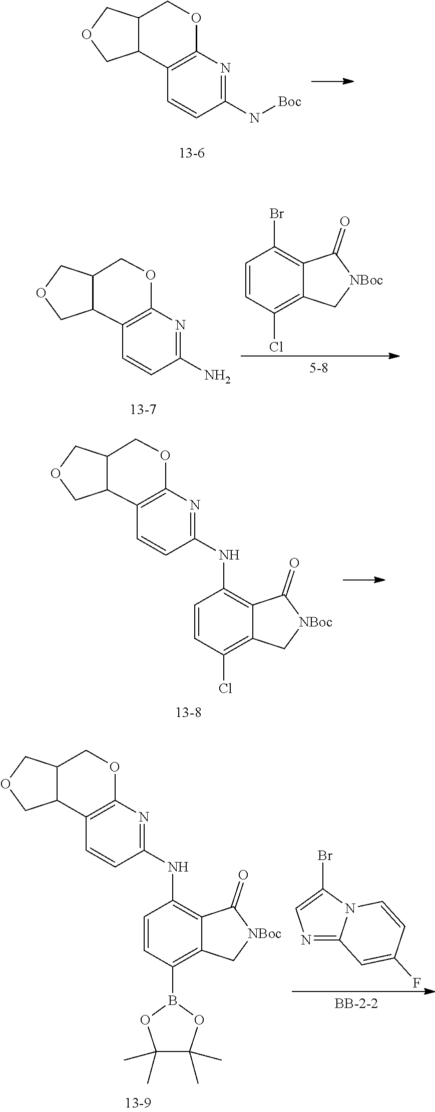 [SMILES: CC(C)(C)OC(=O)Nc1ccc2c(n1)OCC1COCC21 ; Nc1ccc2c(n1)OCC1COCC21 ; CC(C)(C)OC(=O)N1Cc2c(Cl)ccc(Br)c2C1=O ; CC(C)(C)OC(=O)N1Cc2c(Cl)ccc(Nc3ccc4c(n3)OCC3COCC43)c2C1=O ; CC(C)(C)OC(=O)N1Cc2c(B3OC(C)(C)C(C)(C)O3)ccc(Nc3ccc4c(n3)OCC3COCC43)c2C1=O ; Fc1ccn2c(Br)cnc2c1]

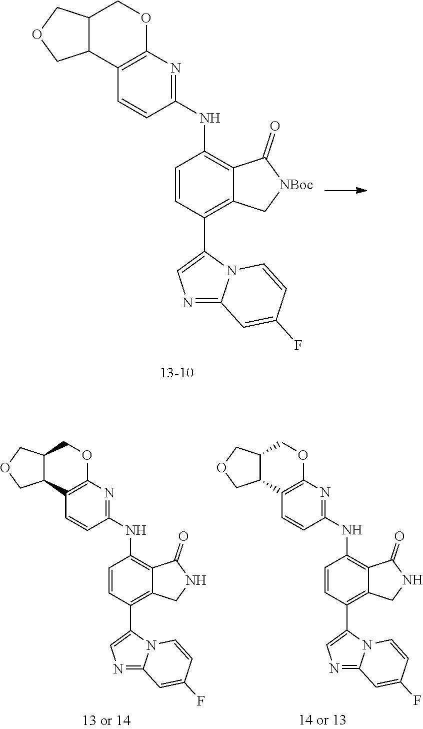 [SMILES: CC(C)(C)OC(=O)N1Cc2c(-c3cnc4cc(F)ccn34)ccc(Nc3ccc4c(n3)OCC3COCC43)c2C1=O ; O=C1NCc2c(-c3cnc4cc(F)ccn34)ccc(Nc3ccc4c(n3)OC[C@@H]3COC[C@H]43)c21 ; O=C1NCc2c(-c3cnc4cc(F)ccn34)ccc(Nc3ccc4c(n3)OC[C@H]3COC[C@@H]43)c21]

### Step 1: Synthesis of Compound 13-2

Compound 13-1 (3 g, 18.97 mmol, 1 eq) was added to tetrahydrofuran (45 mL), and the mixture was cooled to 0° C. Then, sodium hydride (1.14 g, 28.45 mmol, 60% purity, 1.5 eq) was added, and the resulting mixture was stirred for 0.5 hours. Then, N-phenyl-bis(trifluoromethanesulfonimide) (8.13 g, 22.76 mmol, 1.2 eq) was added. The resulting mixture was warmed to 25° C. and reacted for 12 hours. The reaction solution was slowly added to a saturated aqueous solution of ammonium chloride (100 mL), and the resulting mixture was extracted with ethyl acetate (100 mL×3). The organic phases were combined, dried over anhydrous sodium sulfate, and filtered. The filtrate was concentrated under reduced pressure. The resulting crude product was purified by column chromatography (ethyl acetate/petroleum ether=0%-10%) to give compound 13-2. ¹H NMR (400 MHz, CDCl₃) δ ppm 4.85-4.82 (m, 2H) 4.72-4.70 (m, 2H) 4.25-4.20 (m, 2H) 1.28-1.24 (m, 3H).

### Step 2: Synthesis of Compound 13-3

Compound 13-2 (3.9 g, 13.44 mmol, 1 eq) and BB-9 (8.84 g, 20.16 mmol, 1.5 eq) were added to dioxane (100 mL) and water (20 mL). Potassium phosphate (8.56 g, 40.32 mmol, 3 eq) and Pd(dppf)Cl₂ (983.30 mg, 1.34 mmol, 0.1 eq) were added. The resulting mixture was reacted at 100° C. for 3 hours. The reaction solution was added to water (50 mL), and the resulting mixture was extracted with ethyl acetate (50 mL×2). The organic phases were combined, dried over anhydrous sodium sulfate, and filtered. The filtrate was concentrated under reduced pressure. The resulting crude product was purified by column chromatography (ethyl acetate/petroleum ether=0%-4%) to give compound 13-3. MS m/z: 297.0 [M-56]⁺; ¹H NMR (400 MHz, CDCl₃) δ ppm 7.83-7.82 (m, 1H) 7.16 (s, 1H) 5.01 (s, 4H) 4.19-4.14 (m, 2H) 1.54-1.52 (m, 9H) 1.22-1.19 (m, 3H).

### Step 3: Synthesis of Compound 13-4

Compound 13-3 (1.2 g, 3.41 mmol, 1 eq) was added to tetrahydrofuran (30 mL), and the resulting mixture was cooled to 0° C. Then, lithium aluminium hydride (323.15 mg, 8.51 mmol, 2.5 eq) was added, and the resulting mixture was reacted for 2 hours. Water was added dropwise to the reaction solution until the reaction was completely quenched. Anhydrous sodium sulfate was added to dry the mixture, and the mixture was filtered. The filtrate was concentrated under reduced pressure to give compound 13-4. MS m/z: 254.9 [M-56]⁺.

### Step 4: Synthesis of Compound 13-5

Compound 13-4 (1 g, 3.22 mmol, 1 eq) was added to tetrahydrofuran (20 mL), and the mixture was cooled to 0° C. Sodium hydride (257.80 mg, 6.44 mmol, 60% purity, 2 eq) was added, and the resulting mixture was stirred for 0.5 hours. The mixture was heated to 50° C. and reacted for 2 hours. After the reaction, the reaction solution was slowly added to a saturated aqueous solution of ammonium chloride (50 mL). The resulting mixture was extracted with ethyl acetate (50 mL×3). The organic phases were combined, dried over anhydrous sodium sulfate, and filtered. The filtrate was concentrated under reduced pressure to give compound 13-5. MS m/z: 291.0 [M+1]⁺.

### Step 5: Synthesis of Compound 13-6

Palladium on carbon (0.3 g, 10% purity) was added to a dry hydrogenation bottle. Methanol (30 mL) and compound 11-5 (0.8 g, 2.76 mmol, 1 eq) were added. The resulting mixture was reacted at 25° C. under hydrogen (15 psi) for 4 hours. The mixture was filtered, and the filtrate was concentrated under reduced pressure. The resulting crude product was purified by column chromatography (ethyl acetate/petroleum ether=0%-10%) to give compound 13-6. MS m/z: 293.1 [M+1]⁺; ¹H NMR (400 MHz, CDCl₃) δ ppm 7.50-7.48 (m, 1H) 7.38-7.36 (m, 1H) 6.98-6.93 (m, 1H) 4.22-4.21 (m, 2H) 4.20-4.17 (m, 1H) 4.03-3.01 (m, 1H) 3.94-3.92 (m, 1H) 3.67-3.57 (m, 1H) 3.55-3.35 (m, 1H) 2.71-2.68 (m, 1H) 1.44 (s, 9H).

### Step 6: Synthesis of a Hydrochloride of Compound 13-7

Compound 13-6 (0.18 g, 615.74 μmol, 1 eq) was added to hydrochloric acid/methanol (4M, 10 mL), and the resulting mixture was reacted at 25° C. for 2 hours. The reaction solution was concentrated under reduced pressure to give a hydrochloride of compound 13-7. MS m/z: 193.0 [M+1]⁺.

### Step 7: Synthesis of Compound 13-8

Compound 5-8 (0.175 g, 504.90 μmol, 1 eq) and the hydrochloride of compound 13-7 (138.55 mg, 605.88 μmol, 1.20 eq) were added to dioxane (10 mL). Then, potassium carbonate (279.13 mg, 2.02 mmol, 4 eq), 4,5-bis(diphenylphosphino)-9,9-dimethyloxanthene (43.82 mg, 75.73 μmol, 0.15 eq), and Pd₂(dba)₃ (46.23 mg, 50.49 μmol, 0.1 eq) were added. The resulting mixture was reacted at 110° C. for 6 hours. The reaction solution was added to water (30 mL), and the resulting mixture was extracted with ethyl acetate (30 mL×3). The organic phases were combined, dried over anhydrous sodium sulfate, filtered and concentrated. The resulting crude product was purified by column chromatography (ethyl acetate/petroleum ether=0%-25%) to give compound 13-8. MS m/z: 458.1 [M+1]⁺.

### Step 8: Synthesis of Compound 13-9

Compound 13-8 (0.15 g, 327.58 μmol, 1 eq) and bis(pinacolato)diboron (249.55 mg, 982.73 μmol, 3 eq) were added to dioxane (10 mL). Potassium acetate (96.45 mg, 982.73 mol, 3 eq), XPhos Pd G2 (25.77 mg, 32.76 μmol, 0.1 eq), and 2-di-tert-butylphosphino-2′,4′,6′-triisopropylbiphenyl (31.23 mg, 65.52 μmol, 0.2 eq) were added. The resulting mixture was reacted at 110° C. for 3 hours. The reaction solution was added to water (30 mL), and the resulting mixture was extracted with ethyl acetate (30 mL×3). The organic phases were combined, dried over anhydrous sodium sulfate, filtered, and concentrated. The resulting crude product was purified by column chromatography (ethyl acetate/petroleum ether=0%-50%) to give compound 13-9. MS m/z: 550.2 [M+1]⁺.

### Step 9: Synthesis of Compound 13-10

Compound 13-9 (126.48 mg, 230.21 μmol, 1.5 eq) and compound BB-2-2 (33 mg, 153.47 μmol, 1 eq) were added to dioxane (10 mL) and water (2 mL). XPhos Pd G2 (12.08 mg, 15.35 μmol, 0.1 eq) and potassium phosphate (97.73 mg, 460.42 μmol, 3 eq) were added. The resulting mixture was reacted at 105° C. for 3 hours. The reaction solution was added to water (20 mL), and the resulting mixture was extracted with ethyl acetate (20 mL×2). The organic phases were combined, dried over anhydrous sodium sulfate, filtered and concentrated. The resulting crude product was purified by column chromatography (ethyl acetate/petroleum ether=0%-50%) to give compound 13-10. MS m/z: 558.2 [M+1]⁺.

### Step 10: Synthesis of a Trifluoroacetate of Compound 13 and a Trifluoroacetate of Compound 14

Compound 13-10 (70 mg, 125.54 μmol, 1 eq) was added to DCM (10 mL) and trifluoroacetic acid (5 mL). The resulting mixture was reacted at 25° C. for 2 hours. The reaction solution was concentrated under reduced pressure, and separated by SFC (chromatography column: DAICEL CHIRALCEL OJ (250 mm*30 mm, 10 m); mobile phase: [0.1% NH₃H₂O EtOH]; B %: 44%-44%, 12 min). The separated products were concentrated, and 4 drops of trifluoroacetic acid were added, respectively, to give a trifluoroacetate of compound 13 and a trifluoroacetate of compound 14.

The trifluoroacetate of compound 13: MS m/z: 458.1 [M+1]⁺; ¹H NMR (400 MHz, CD₃OD) δ ppm 8.82 (d, J=8.4 Hz, 1H) 8.69-8.67 (m, 1H) 8.21 (s, 1H) 7.86-7.84 (m, 1H) 7.76-7.74 (m, 1H) 7.60 (d, J=8.0 Hz, 1H) 7.48-7.46 (m, 1H) 6.66 (d, J=8.0 Hz, 1H) 4.42 (s, 2H) 4.35-4.34 (m, 1H) 4.27-4.25 (m, 1H) 4.11-4.09 (m, 2H) 3.79 (m, 1H) 3.71-3.74 (s, 1H) 3.52-3.50 (m, 1H) 2.86-2.83 (m, 1H). ee %=100%, retention time: 1.643 min.

The trifluoroacetate of compound 14: MS m/z: 458.1 [M+1]⁺; ¹H NMR (400 MHz, CD₃OD) δ ppm 8.83 (d, J=8.4 Hz, 1H) 8.67 (m, 1H) 8.20 (s, 1H) 7.84 (d, J=5.4 Hz, 1H) 7.77-7.75 (m, 1H) 7.60 (d, J=7.8 Hz, 1H) 7.47-7.44 (m, 1H) 6.67 (d, J=7.8 Hz, 1H) 4.42 (s, 2H) 4.37-4.35 (m, 1H) 4.27 (s, 1H) 4.11-4.09 (m, 2H) 3.81-3.80 (m, 1H) 3.79-3.71 (m, 1H) 3.52-3.50 (m, 1H) 2.85-2.83 (m, 1H). ee %=98.42%, retention time: 1.827 min.

The analysis method of ee % detection: chromatography column: Chiralcel OJ-3, 50×4.6 mm I.D., 3 μm; mobile phase: A: carbon dioxide; B: ethanol (0.1% IPAm, v/v).

### Example 15

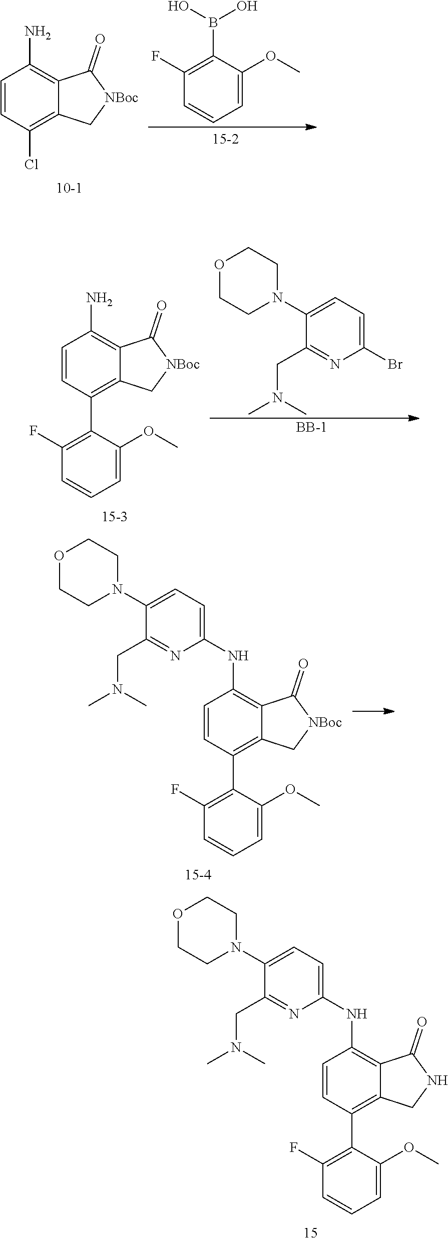

### Step 1: Synthesis of Compound 15-3

Compound 10-1 (0.2 g, 707.41 μmol, 1 eq) and compound 15-2 (240.44 mg, 1.41 mmol, 2 eq) were added to dioxane (4 mL) and water (0.8 mL). XPhos Pd G2 (55.66 mg, 70.74 mol, 0.1 eq) and potassium phosphate (450.48 mg, 2.12 mmol, 3 eq) were added. The resulting mixture was reacted at 100° C. for 2 hours. The reaction solution was added to water (30 mL), and the resulting mixture was extracted with ethyl acetate (30 mL×2). The organic phases were combined, dried over anhydrous sodium sulfate, filtered and concentrated. The resulting crude product was separated by column chromatography (ethyl acetate/petroleum ether=0%-15%) to give compound 15-3. MS m/z: 273.0 [M-100]⁺; ¹H NMR (400 MHz, DMSO-d₆) δ ppm 7.41-7.37 (m, 1H) 7.21-7.19 (m, 1H) 6.99-6.89 (m 2H) 6.70-6.69 (m, 1H) 6.51 (s, 2H) 4.36-4.29 (m, 2H) 3.75 (s, 3H) 1.48 (s, 9H).

### Step 2: Synthesis of Compound 15-4

Compound 15-3 (0.18 g, 483.37 μmol, 1 eq) and compound BB-1 (174.12 mg, 580.04 gmol, 1.2 eq) were added to dioxane (10 mL). Cesium carbonate (472.47 mg, 1.45 mmol, 3 eq), 4,5-bis(diphenylphosphino)-9,9-dimethylxanthene (41.95 mg, 72.50 μmol, 0.15 eq), and palladium acetate (10.85 mg, 48.34 μmol, 0.1 eq) were added. The resulting mixture was reacted at 110° C. for 3 hours. The reaction solution was added to water (50 mL), and the resulting mixture was extracted with ethyl acetate (50 mL×3). The organic phases were combined, dried over anhydrous sodium sulfate, filtered, and concentrated to give compound 15-4. MS m/z: 592.3 [M+1]⁺.

### Step 3: Synthesis of a Trifluoroacetate of Compound 15

Compound 15-4 (0.4 g, 676.05 μmol, 1 eq) was added to hydrochloric acid/methanol (4M, 20 mL), and the resulting mixture was reacted at 25° C. for 4 hours. The reaction solution was concentrated under reduced pressure, and purified by prep-HPLC (chromatography column: Phenomenex Lμna 80*30 mm*3 μm; mobile phase: [water (TFA)-acetonitrile]; acetonitrile %: 10%-50%, 8 min) to give a trifluoroacetate of compound 15. MS m/z: 492.2 [M+1]⁺; ¹H NMR (400 MHz, CD₃OD) δ ppm 8.26 (d, J=8.4 Hz, 1H) 7.81 (d, J=8.8 Hz, 1H) 7.48-7.40 (m, 2H) 7.21 (d, J=8.8 Hz, 1H) 6.97 (d, J=8.4 Hz, 1H) 6.87 (t, J=8.8 Hz, 1H) 4.62 (s, 2H) 4.28-4.19 (m, 2H) 3.90-3.88 (m, 4H) 3.81 (s, 3H) 3.05 (s, 6H) 2.93-2.91 (m, 4H).

### Example 16

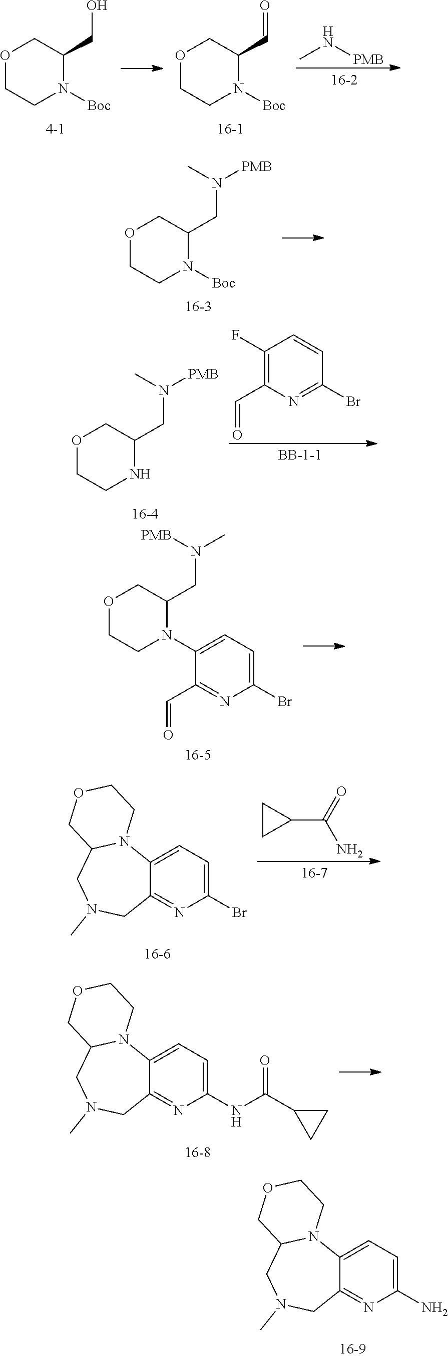

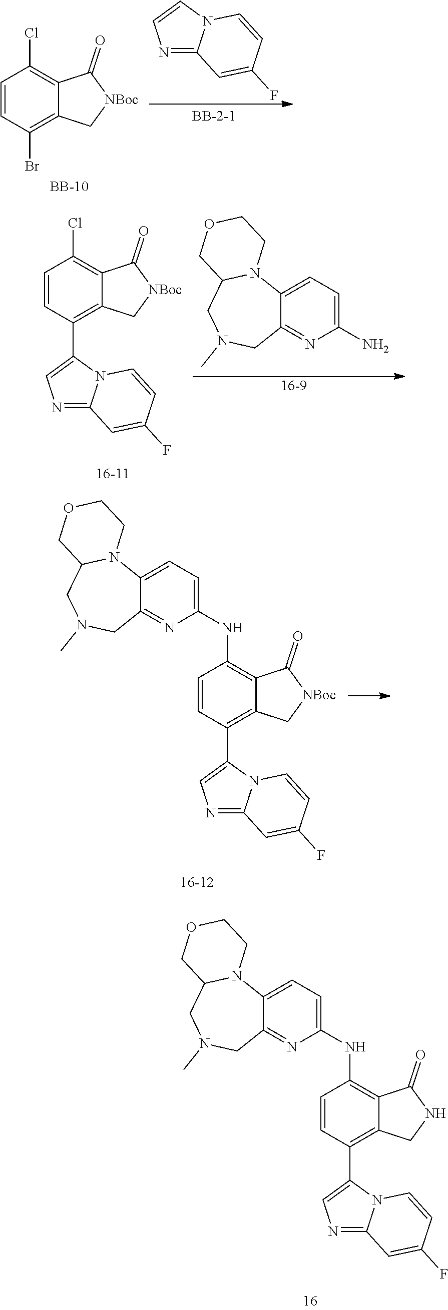

### Step 1: Synthesis of Compound 16-1

Compound 4-1 (10 g, 46.03 mmol) was dissolved in DCM (10 mL) at 0° C. under nitrogen, and Dess-Martin periodinane (23.43 g, 55.23 mmol, 17.10 mL) was added. The resulting mixture was warmed to 20° C. and reacted for 16 hours. The mixture was filtered, and the solid was washed with dichloromethane (20 mL). The filtrate was poured into a saturated sodium sulfite solution (200 mL), and the mixture was filtered. The pH of the filtrate was adjusted to 8 with a saturated sodium bicarbonate solution (200 mL). The organic phase was washed with saturated brine (100 mL), dried over anhydrous sodium sulfate, vacuum filtered, and concentrated under reduced pressure. The resulting crude product was purified by column chromatography (ethyl acetate/petroleum ether=0%-15%) to give compound 16-1. ¹H NMR (400 MHz, DMSO-d₆) δ 9.61-9.59 (m, 1H), 4.51-4.34 (m, 2H), 3.79-3.68 (m, 1H), 3.65-3.52 (m, 2H), 3.40-3.35 (m, 1H), 3.11-2.79 (m, 1H), 1.46-1.37 (m, 9H).

### Step 2: Synthesis of Compound 16-3

Compound 16-1 (2.89 g, 13.43 mmol) was added to DCM (60 mL) under nitrogen. Compound 16-2 (2.44 g, 16.11 mmol) and acetic acid (80.63 mg, 1.34 mmol, 76.79 μL) were added. The resulting mixture was reacted at 20° C. for 1 hour. Then, sodium acetate borohydride (5.69 g, 26.85 mmol) was added, and the mixture was reacted at 20° C. for 12 hours. A saturated aqueous solution of sodium bicarbonate (60 mL) was added to quench the reaction. The resulting mixture was extracted with dichloromethane (50 mL×3). The organic phases were combined, washed with saturated brine (50 mL), dried over anhydrous sodium sulfate, vacuum filtered, and concentrated under reduced pressure. The resulting crude product was purified by column chromatography (ethyl acetate/petroleum ether=0%-30%) to give compound 16-3. MS m/z: 351.2 [M+1]⁺; ¹H NMR (400 MHz, DMSO-d₆) δ 7.18 (d, J=8.4 Hz, 2H), 6.85 (d, J=8.8 Hz, 2H), 3.82-3.73 (m, 2H), 3.69-3.65 (m, 4H), 3.54-3.51 (m, 1H), 3.48-3.36 (m, 3H), 3.30-3.24 (m, 1H), 2.85-2.68 (m, 1H), 2.61-2.56 (m, 1H), 2.43-2.38 (m, 1H), 2.14 (s, 3H), 1.40 (s, 9H).

### Step 3: Synthesis of Compound 16-4

Compound 16-3 (1.7 g, 4.85 mmol) was dissolved in DCM (2 mL) under nitrogen, and hydrochloric acid/methanol (4 M, 50 mL) was added. The resulting mixture was reacted at 20° C. for 6 hours. The solvent was concentrated under reduced pressure. Dichloromethane (20 mL) and aqueous ammonia (3 mL) were added. The resulting mixture was dried over anhydrous sodium sulfate, vacuum filtered, and concentrated under reduced pressure to give compound 16-4. MS m/z: 251.1 [M+1]⁺; ¹H NMR (400 MHz, DMSO-d₆) δ 7.20 (d, J=8.8 Hz, 2H), 6.87 (d, J=8.4 Hz, 2H), 3.73-3.65 (m, 5H), 3.44-3.33 (m, 3H), 3.09-3.01 (m, 1H), 2.99-2.92 (m, 1H), 2.85-2.74 (m, 2H), 2.33-2.25 (m, 1H), 2.21-2.15 (m, 1H), 2.11 (s, 3H).

### Step 4: Synthesis of Compound 16-5

Compound BB-1-1 (600 mg, 2.94 mmol) was dissolved in DMF (12 mL) under nitrogen. Compound 16-4 (809.92 mg, 3.24 mmol) and DIEA (1.14 g, 8.82 mmol, 1.54 mL) were added. The resulting mixture was reacted at 100° C. for 6 hours. The mixture was cooled, and water (20 mL) was added to quench the reaction. The resulting mixture was extracted with ethyl acetate (20 mL×4). The organic phase was washed with saturated brine (20 mL), dried over anhydrous sodium sulfate, vacuum filtered, and concentrated under reduced pressure. The resulting crude product was purified by column chromatography (ethyl acetate/petroleum ether=0%-20-25%) to give compound 16-5. MS m/z: 434.1 [M+1]⁺; ¹H NMR (400 MHz, DMSO-d₆) δ 9.83 (s, 1H), 7.71 (d, J=8.8 Hz, 1H), 7.59 (d, J=8.8 Hz, 1H), 6.92-6.86 (m, 2H), 6.83-6.78 (m, 2H), 3.92-3.76 (m, 3H), 3.73 (s, 3H), 3.59-3.55 (m, 1H), 3.55-3.49 (m, 1H), 3.29-3.27 (m, 1H), 3.24-3.19 (m, 2H), 2.95-2.87 (m, 1H), 2.64-2.62 (m, 1H), 2.42-2.37 (m, 1H), 1.81 (s, 3H).

### Step 5: Synthesis of Compound 16-6

Compound 16-5 (1.24 g, 2.85 mmol) was dissolved in trifluoroacetic acid (15 mL) under nitrogen, and the resulting mixture was reacted at 60° C. for 2 hours. The reaction solution was cooled and concentrated under reduced pressure. DCM (30 mL) and NaBH(OAc)₃ (1.82 g, 8.56 mmol) were added, and the resulting mixture was reacted at 20° C. for 2 hours. Saturated sodium carbonate solution (30 mL) and saturated sodium hydroxide solution (5 mL) were added to quench the reaction, and the resulting mixture was extracted with dichloromethane (20 mL×5). The organic phase was washed with saturated brine (20 mL), dried over anhydrous sodium sulfate, vacuum filtered, and concentrated under reduced pressure. The resulting crude product was purified by column chromatography (methanol/ethyl acetate=0%-5%) to give compound 16-6. MS m/z: 298.0 [M+1]⁺; ¹H NMR (400 MHz, DMSO-d₆) δ 7.36-7.33 (m, 2H), 3.94-3.92 (m, 1H), 3.78-3.62 (m, 4H), 3.46-3.40 (m, 1H), 3.38-3.35 (m, 1H), 3.25-3.23 (m, 2H), 3.01-2.95 (m, 1H), 2.58-2.52 (m, 1H), 2.33 (s, 3H).

### Step 6: Synthesis of Compound 16-8

Compound 16-6 (590 mg, 1.98 mmol) was dissolved in dioxane (12 mL) under nitrogen. Compound 16-7 (336.79 mg, 3.96 mmol), cesium carbonate (1.93 g, 5.94 mmol), Xantphos (228.98 mg, 395.74 μmol), and Pd₂(dba)₃ (181.19 mg, 197.87 μmol) were added. The resulting mixture was reacted at 110° C. for 2 hours. The mixture was cooled and vacuum filtered. The filter cake was washed with dichloromethane (20 mL). The filtrate was concentrated under reduced pressure. The resulting crude product was purified by column chromatography (methanol/ethyl acetate=0%-5%) to give compound 16-8. MS m/z: 303.2 [M+1]⁺; ¹H NMR (400 MHz, DMSO-d₆) δ 10.55 (s, 1H), 7.86 (d, J=8.8 Hz, 1H), 7.37 (d, J=8.8 Hz, 1H), 3.87-3.72 (m, 3H), 3.69-3.65 (m, 2H), 3.45-3.39 (m, 1H), 3.19-3.16 (m, 3H), 2.92-2.86 (m, 1H), 2.55-2.51 (m, 1H), 2.29 (s, 3H), 2.03-1.90 (m, 1H), 0.80-0.71 (m, 4H).

### Step 7: Synthesis of Compound 16-9

Compound 16-8 (500 mg, 1.65 mmol) was dissolved in methanol (10 mL) and water (2.5 mL) under nitrogen. Sodium hydroxide (661.39 mg, 16.54 mmol) was added, and the resulting mixture was reacted at 80° C. for 16 hours. Sodium hydroxide (330.70 mg, 8.27 mmol) was then added, and the resulting mixture was reacted at 80° C. for 4 hours. The solvent was concentrated under reduced pressure, and the resulting mixture was extracted with dichloromethane (20 mL×5). The organic phase was washed with saturated brine (20 mL), dried over anhydrous sodium sulfate, vacuum filtered, and concentrated under reduced pressure to give compound 16-9. MS m/z: 235.2 [M+1]⁺; ¹H NMR (400 MHz, DMSO-d₆) δ 7.08 (d, J=8.8 Hz, 1H), 6.28 (d, J=8.4 Hz, 1H), 5.47 (s, 2H), 3.73-3.56 (m, 5H), 3.48-3.41 (m, 1H), 3.20-3.14 (m, 1H), 3.06-2.95 (m, 2H), 2.92-2.87 (m, 1H), 2.47-2.45 (m, 1H), 2.26 (s, 3H).

### Step 8: Synthesis of Compound 16-11

Compound BB-10 (8.3 g, 23.95 mmol) was dissolved in DMF (83 mL) under nitrogen. BB-2-1 (3.26 g, 23.95 mmol), potassium acetate (4.70 g, 47.89 mmol), and Pd(dppf)Cl₂ (1.75 g, 2.39 mmol) were added. The resulting mixture was reacted at 120° C. for 2 hours. The mixture was cooled and filtered. The filtrate was poured into water (1000 mL), and the resulting mixture was extracted with ethyl acetate (1000 mL). The organic phase was dried over anhydrous sodium sulfate, filtered, and concentrated to give a crude product. The crude product was purified by column chromatography (petroleum ether:ethyl acetate=0%-50%) to give compound 16-11. MS m/z: 402.1 [M+1]⁺; ¹H NMR (400 MHz, CDCl₃) δ 8.04-7.96 (m, 1H), 7.73-7.70 (m, 1H), 7.67-7.57 (m, 2H), 7.41-7.32 (m, 1H), 6.83-6.72 (m, 1H), 4.64 (s, 2H), 1.58 (s, 9H).

### Step 9: Synthesis of Compound 16-12

Compound 16-11 (610 mg, 1.52 mmol) was dissolved in dioxane (12 mL) under nitrogen. Compound 16-9 (391.25 mg, 1.67 mmol), cesium carbonate (1.48 g, 4.55 mmol), Xantphos (175.68 mg, 303.62 μmol), and palladium acetate (34.08 mg, 151.81 μmol) were added. The resulting mixture was reacted at 110° C. for 2 hours. The mixture was cooled and vacuum filtered. The residue was washed with dichloromethane (20 mL). The filtrate was concentrated under reduced pressure. The resulting crude product was purified by column chromatography (methanol/ethyl acetate=0%-30%) to give compound 16-12. MS m/z: 600.3 [M+1]⁺; ¹H NMR (400 MHz, DMSO-d₆) δ 9.65 (s, 1H), 8.66 (d, J=8.4 Hz, 1H), 8.43-8.36 (m, 1H), 7.82 (s, 1H), 7.78 (d, J=8.4 Hz, 1H), 7.55-7.52 (m, 1H), 7.41 (d, J=8.8 Hz, 1H), 7.02-6.95 (m, 2H), 4.73 (s, 2H), 3.97-3.88 (m, 1H), 3.83-3.79 (m, 1H), 3.74-3.72 (m, 1H), 3.69-3.63 (m, 1H), 3.49-3.45 (m, 1H), 3.29-3.20 (m, 2H), 3.17 (d, J=5.2 Hz, 2H), 3.09-2.98 (m, 1H), 2.58-2.54 (m, 1H), 2.36 (s, 3H), 1.51 (s, 9H).

### Step 10: Synthesis of a Trifluoroacetate of Compound 16

Compound 16-12 (742 mg, 1.24 mmol) was dissolved in DCM (10 mL) under nitrogen, and trifluoroacetic acid (7.70 g, 67.53 mmol, 5 mL) was added. The resulting mixture was reacted at 20° C. for 1 hour. The solvent was concentrated under reduced pressure to give a crude product. The crude product was purified by prep-HPLC (chromatography column: Phenomenex 1 μna C18 250*50 mm*10 μm; mobile phase: [water (TFA)-acetonitrile]; acetonitrile %: 5%-45%, 10 min) to give a trifluoroacetate of compound 16. MS m/z: 500.2 [M+1]⁺; ¹H NMR (400 MHz, DMSO-d₆) δ 10.10 (s, 1H), 8.89 (s, 1H), 8.71-8.56 (m, 2H), 8.24 (s, 1H), 7.94-7.92 (m, 1H), 7.72 (d, J=8.4 Hz, 1H), 7.59 (d, J=8.0 Hz, 1H), 7.44-7.37 (m, 1H), 7.13 (d, J=8.0 Hz, 1H), 4.86-4.67 (m, 1H), 4.56-4.53 (m, 1H), 4.39 (s, 2H), 3.78-3.64 (m, 6H), 3.42-3.36 (m, 1H), 3.31-3.17 (m, 1H), 2.97 (s, 3H), 2.58-2.56 (m, 1H).

### Example 17

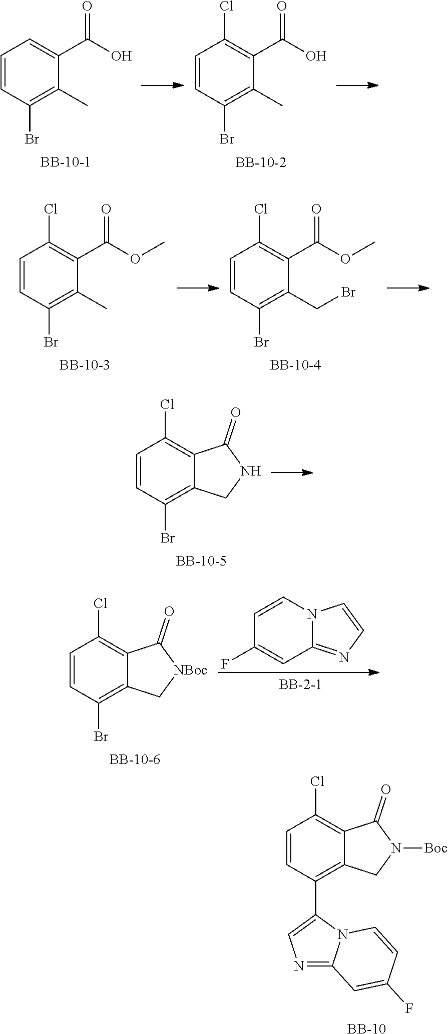

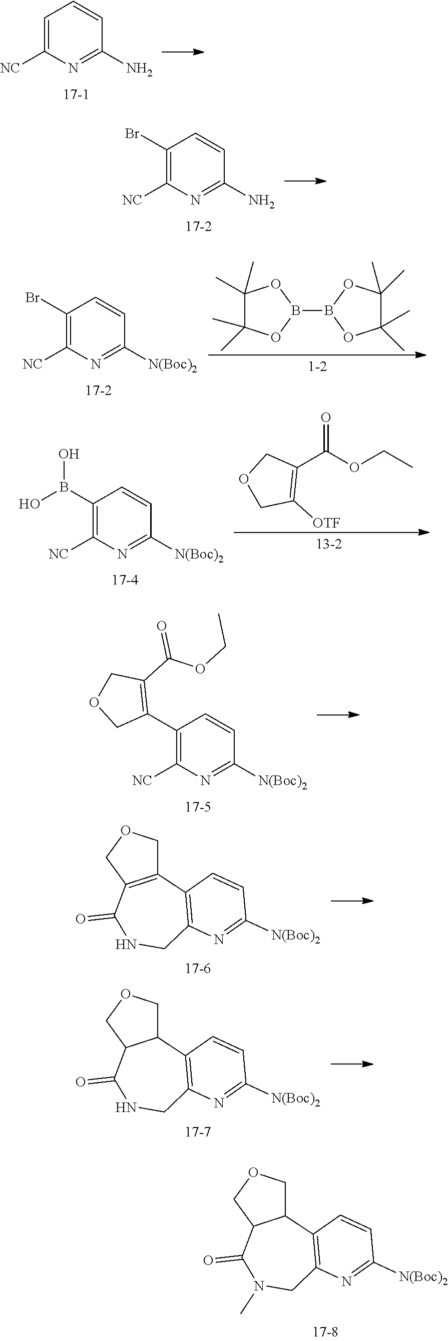

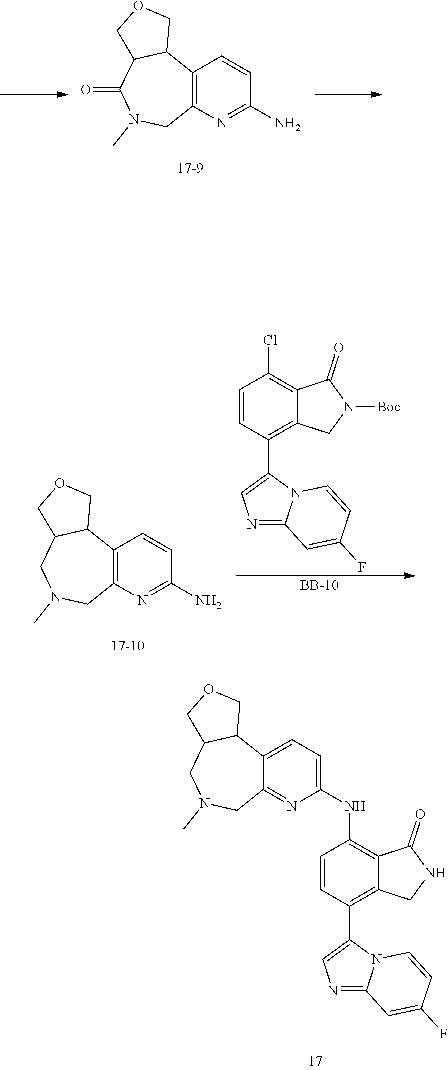

### Step 1: Synthesis of Compound BB-10-2

Compound BB-10-1 (23.4 g, 108.82 mmol) was dissolved in DMF (230 mL). N-chlorosuccinimide (17.44 g, 130.58 mmol) and palladium acetate (12.22 g, 54.41 mmol) were added. The reaction system was purged with nitrogen three times. The reaction solution was stirred at 110° C. for 16 hours. The reaction solution was poured into water (1 L), and ethyl acetate (300 mL) was added. The resulting mixture was stirred evenly and then filtered. The layers of the filtrate were separated. The aqueous phase was extracted with ethyl acetate (200 mL×2). The organic phases were combined, dried over anhydrous sodium sulfate, filtered and concentrated. The crude product was purified by column chromatography (ethyl acetate/petroleum ether=1%-25%) to give compound BB-10-2. MS m/z: 231.0 [M-17]⁺; ¹H NMR (400 MHz, CDCl₃) δ 11.38 (m, 1H), 7.56 (d, J=8.4 Hz, 1H), 7.15 (d, J=8.4 Hz, 1H), 2.48 (s, 3H).

### Step 2: Synthesis of Compound BB-10-3

Compound BB-10-2 (23.7 g, 94.99 mmol) was dissolved in DMF (237 mL), and anhydrous potassium carbonate (32.82 g, 237.49 mmol) was added. The resulting mixture was stirred at 20° C. for 30 minutes, and then iodomethane (20.23 g, 142.49 mmol, 8.87 mL) was added dropwise. The reaction solution was stirred at 20° C. for another 16 hours. The reaction solution was poured into 2% dilute aqueous ammonia (100 mL) to quench the reaction. Water (1000 mL) was added to dilute the mixture. The aqueous phase was extracted with ethyl acetate (200 mL×3). The organic phases were combined, dried over anhydrous sodium sulfate, filtered and concentrated. The crude product was purified by column chromatography (ethyl acetate/petroleum ether=0%-10%) to give compound BB-10-3. MS m/z: 263.0 [M+1]⁺; ¹H NMR (400 MHz, CDCl₃) δ 7.51 (d, J=8.4 Hz, 1H), 7.11 (d, J=8.8 Hz, 1H), 3.96 (s, 3H), 2.36 (s, 3H).

### Step 3: Synthesis of Compound BB-10-4

Compound BB-10-3 (7.7 g, 29.22 mmol) was dissolved in 1,2-dichloroethane (150 mL). N-bromosuccinimide (5.72 g, 32.14 mmol) and benzoyl peroxide (707.80 mg, 2.92 mmol) were added. The reaction system was purged with nitrogen three times, and the reaction solution was stirred at 110° C. under nitrogen for 40 hours. The reaction solution was poured into water (200 mL). The layers were separated, and the aqueous phase was extracted with dichloromethane (50 mL×2). The organic phases were combined, dried over anhydrous sodium sulfate, filtered and concentrated to give compound BB-10-4. MS m/z: 340.9 [M+1]⁺.

### Step 4: Synthesis of Compound BB-10-5

Compound BB-10-4 (10 g, 29.20 mmol) was dissolved in ammonia in methanol (7 M, 208.60 mL), and the reaction solution was stirred at 20° C. for 16 hours. The reaction solution was directly filtered, and the filter cake was washed with methanol (50 mL). The filter cake was collected, concentrated and dried to give compound BB-10-5. MS m/z: 246.0 [M+1]⁺; ¹H NMR (400 MHz, DMSO-d₆) δ 8.90 (br s, 1H), 7.79 (d, J=8.4 Hz, 1H), 7.43 (d, J=8.4 Hz, 1H), 4.44 (s, 2H).

### Step 5: Synthesis of Compound BB-10-6

Compound BB-10-5 (5.3 g, 21.50 mmol) was dissolved in tetrahydrofuran (80 mL). Di-tert-butyl dicarbonate (7.04 g, 32.25 mmol, 7.41 mL) and 4-dimethylaminopyridine (3.15 g, 25.80 mmol) were added, and the reaction solution was stirred at 20° C. for 16 hours. The reaction solution was poured into water (200 mL), and the layers were separated. The aqueous phase was extracted with ethyl acetate (50 mL×2). The organic phases were combined, dried over anhydrous sodium sulfate, filtered and concentrated. The resulting crude product was purified by column chromatography (ethyl acetate/petroleum ether=5%-25%) to give compound BB-10-6. MS m/z: 290.0 [M-55]⁺; ¹H NMR (400 MHz, CDCl₃) δ 7.66 (d, J=8.4 Hz, 1H), 7.34 (d, J=8.4 Hz, 1H), 4.63 (s, 2H), 1.61 (s, 9H).

### Step 6: Synthesis of Compound BB-10

Compound BB-10-6 (5 g, 14.43 mmol) was dissolved in N-methyl-2-pyrrolidone (50 mL), and the reaction system was purged with nitrogen three times. Compound BB-2-1 (1.96 g, 14.43 mmol), potassium acetate (2.83 g, 28.85 mmol), and Pd(dppf)Cl₂ (1.06 g, 1.44 mmol) were added, and the resulting mixture was reacted at 120° C. for 2 hours. The reaction solution was poured into water (200 mL), and solids were precipitated. The mixture was filtered, and the filter cake was collected and dried. The resulting crude product was purified by column chromatography (ethyl acetate/petroleum ether=50%-100%) to give compound BB-10. MS m/z: 402.2 [M+1]⁺; ¹H NMR (400 MHz, CDCl₃) δ 7.99-7.96 (m, 1H), 7.73 (s, 1H), 7.69-7.64 (m, 2H), 7.40-7.35 (m, 1H), 6.84-6.78 (m, 1H), 4.66 (s, 2H), 1.59 (s, 9H).

### Step 7: Synthesis of Compound 17-2

Compound 17-1 (30 g, 251.84 mmol) was dissolved in DCM (300 mL) and methanol (300 mL), and then tetrabutylammonium tribromide (145.72 g, 302.21 mmol) was added. The reaction solution was reacted at 25° C. for 2 hours. The reaction solution was concentrated under reduced pressure, and then dichloromethane (600 mL) was added. The resulting mixture was stirred for 0.5 hours and then filtered. The filtrate was concentrated to give a crude product. The crude product was dissolved in dichloromethane (50 mL). The reaction solution was reacted at 25° C. for 2 hours and filtered. The filter cake was concentrated to give compound 17-2. MS m/z: 197.9 [M+1]⁺.

### Step 8: Synthesis of Compound 17-3

Compound 17-2 (8 g, 40.40 mmol) was dissolved in DCM (100 mL). Then, 4-dimethylaminopyridine (4.94 g, 40.40 mmol), di-tert-butyl dicarbonate (26.45 g, 121.20 mmol, 27.84 mL), and DIEA (15.66 g, 121.20 mmol, 21.11 mL) were added. The reaction solution was reacted at 25° C. for 16 hours. The reaction solution was poured into a saturated aqueous solution of ammonium chloride (200 mL). Then, the mixture was extracted with dichloromethane (100 mL×3). The organic phase was dried over anhydrous sodium sulfate, filtered and concentrated to give a crude product. The crude product was purified by column chromatography (ethyl acetate/petroleum ether=10%-25%) to give compound 17-3. MS m/z: 241.8 [M-155]⁺; ¹H NMR (400 MHz, CDCl₃) δ 7.98 (d, J=8.4 Hz, 1H), 7.56 (d, J=8.8 Hz, 1H), 1.50 (s, 18H).

### Step 9: Synthesis of Compound 17-4

Compound 17-3 (8.3 g, 20.84 mmol) was dissolved in 1,4-dioxane (100 mL). Then, potassium acetate (5.11 g, 52.10 mmol) and compound 1-2 (7.94 g, 31.26 mmol) were added. The reaction system was purged with nitrogen, and then Pd(dppf)Cl₂ (1.52 g, 2.08 mmol) was added. The reaction solution was reacted at 80° C. for 2 hours. The reaction solution was poured into a saturated aqueous solution of ammonium chloride (50 mL), and then the mixture was extracted with ethyl acetate (50 mL×3). The organic phase was dried over anhydrous sodium sulfate, filtered and concentrated to give compound 17-4. MS m/z: 207.9 [M-155]⁺.

### Step 10: Synthesis of Compound 17-5

Compound 17-4 (7.5 g, 20.65 mmol) and 13-2 (8.99 g, 30.98 mmol) were dissolved in tetrahydrofuran (100 mL). Then, anhydrous potassium carbonate (7.14 g, 51.63 mmol) and Pd(PPh₃)₄ (2.39 g, 2.07 mmol) were added. The reaction solution was reacted at 65° C. for 1 hour. The reaction solution was poured into a saturated aqueous solution of ammonium chloride (150 mL), and then the mixture was extracted with ethyl acetate (150 mL×3). The organic phase was dried over anhydrous sodium sulfate, filtered and concentrated to give a crude product. The crude product was purified by column chromatography (ethyl acetate/petroleum ether=10%-50%) to give compound 17-5. MS m/z: 304.0 [M-155]⁺; ¹H NMR (400 MHz, CDCl₃) δ 7.76-7.70 (m, 2H), 5.11-5.04 (m, 4H), 4.18-4.12 (m, 2H), 1.53 (s, 18H), 1.20-1.16 (m, 3H).

### Step 11: Synthesis of Compound 17-6

Raney nickel (2 g) was added to a dry hydrogenation bottle, and washed with methanol three times. Then, a solution of compound 17-5 (5.1 g, 11.10 mmol) in methanol (40 mL) and triethylamine (10 mL) was added. The reaction system was purged with hydrogen three times. Then, the reaction solution was stirred at 25° C. under 40 Psi of hydrogen for 16 hours. The reaction solution was directly filtered, and the filtrate was concentrated to give a crude product. The crude product was purified by column chromatography (ethyl acetate/petroleum ether=10%-100%) to give compound 17-6. MS m/z: 418.1 [M+1]⁺.

### Step 12: Synthesis of Compound 17-7

Wet palladium on carbon (200 mg, 10% purity) was added to a dry vial. Then, a solution of compound 17-6 (1.5 g, 3.59 mmol) in methanol (20 mL) was slowly added under nitrogen. The reaction system was purged with hydrogen three times, and then the reaction solution was stirred at 20° C. under 20 Psi of hydrogen for 16 hours. The reaction solution was filtered, and the filtrate was concentrated to give compound 17-7. MS m/z: 420.2 [M+1]⁺.

### Step 13: Synthesis of Compound 17-8

Compound 17-7 (1 g, 2.38 mmol) was dissolved in tetrahydrofuran (20 mL) in a dry vial. Sodium hydride (286.08 mg, 7.15 mmol, 60% purity) was added at 0° C. under nitrogen. The resulting mixture was stirred for 30 minutes. Then, iodomethane (507.57 mg, 3.58 mmol, 222.62 μL) was added, and the reaction solution was stirred at 20° C. for 2 hours. The reaction solution was poured into a saturated aqueous solution of ammonium chloride (20 mL). The aqueous phase was extracted with ethyl acetate (10 mL×2). The organic phases were combined, dried over anhydrous sodium sulfate, filtered and concentrated. The crude product was purified by column chromatography (ethyl acetate/petroleum ether=15%-100) to give compound 17-8. MS m/z: 434.3 [M+1]⁺.

### Step 14: Synthesis of Compound 17-9

Compound 17-8 (700 mg, 1.61 mmol) was dissolved in DCM (20 mL) in a dry vial, and trifluoroacetic acid (9.21 g, 80.74 mmol, 5.98 mL) was added. The reaction solution was stirred at 25° C. for 16 hours. The reaction solution was directly concentrated to give a crude product. The crude product was purified by a preparative thin layer chromatography silica gel plate (methanol/dichloromethane=10%) to give compound 17-9. MS m/z: 234.2 [M+1]⁺.

### Step 15: Synthesis of Compound 17-10

Compound 17-9 (95 mg, 407.26 μmol) was dissolved in tetrahydrofuran (5 mL) in a dry vial. Lithium aluminium hydride (77.28 mg, 2.04 mmol) was added at 0° C. under nitrogen, and the reaction solution was stirred at 60° C. for 2 hours. The reaction solution was cooled to 20° C. Then, methanol (10 mL) was added dropwise to quench the reaction, and then the mixture was concentrated. The crude product was purified by column chromatography (methanol/dichloromethane=0%-15%) to give compound 17-10. MS m/z: 220.0 [M+1]⁺.

### Step 16: Synthesis of a Trifluoroacetate of Compound 17

Compound 17-10 (65 mg, 296.42 μmol) and compound BB-10 (120 mg, 298.64 mol) were dissolved in 1,4-dioxane (10 mL) in a dry vial, and cesium carbonate (291.91 mg, 895.93 μmol) was added. Pd₂(dba)₃ (54.69 mg, 59.73 μmol) and Xantphos (43.20 mg, 74.66 mol) were added under nitrogen. The reaction solution was stirred at 110° C. under nitrogen for 2 hours. The reaction solution was directly filtered, and the filtrate was concentrated. The resulting crude product was purified by a preparative thin layer chromatography silica gel plate (methanol/dichloromethane=10%) to give a crude product. The crude product was purified by prep-HPLC (chromatography column: Phenomenex Luna 80*30 mm*3 μm; mobile phase: [water (TFA)-acetonitrile]; acetonitrile %: 1%-30%, 8 min) to give a trifluoroacetate of compound 17. MS m/z: 485.3 [M+1]⁺; ¹H NMR (400 MHz, DMSO-d₆) δ 10.23 (s, 1H), 8.91 (s, 1H), 8.74 (s, 1H), 8.54 (s, 1H), 8.11 (s, 1H), 7.80-7.78 (m, 1H), 7.39 (d, J=8.8 Hz, 1H), 7.57 (d, J=8.4 Hz, 1H), 7.27-7.24 (m, 1H), 7.19-7.17 (m, 1H), 4.91-4.40 (m, 5H), 4.19-4.14 (m, 2H), 3.79-3.69 (m, 4H), 3.41-3.00 (m, 2H), 2.83-2.67 (m, 2H).

### Example 18

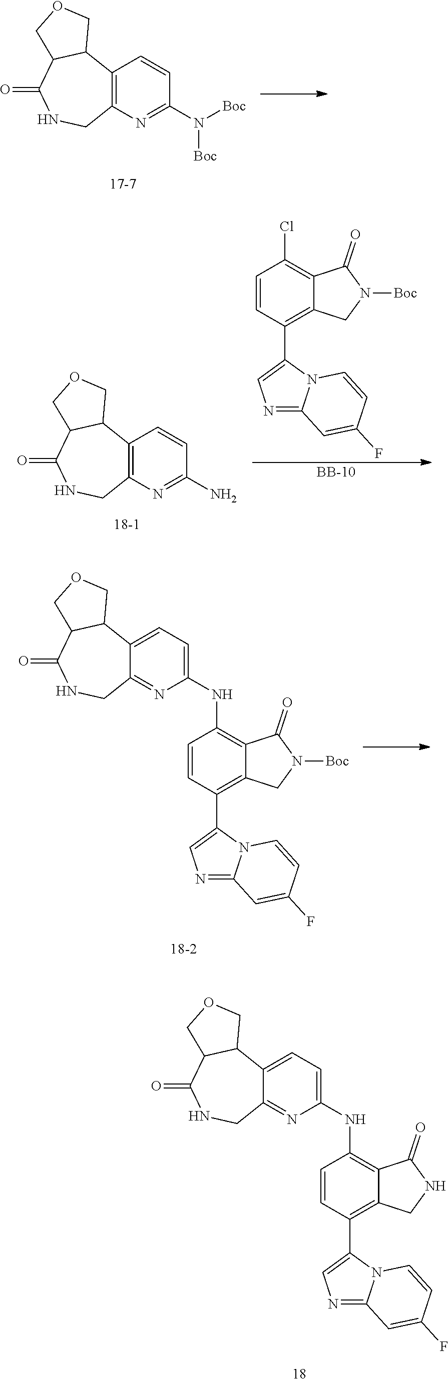

### Step 1: Synthesis of a Trifluoroacetate of Compound 18-1

Compound 17-7 (200 mg, 476.79 μmol) was dissolved in DCM (6 mL) in a dry vial, and trifluoroacetic acid (2.72 g, 23.84 mmol, 1.77 mL) was added. The reaction solution was stirred at 25° C. for 16 hours. The reaction solution was concentrated to give a trifluoroacetate of compound 18-1. MS m/z: 220.2 [M+1]⁺.

### Step 2: Synthesis of Compound 18-2

The trifluoroacetate of compound 18-1 (109.12 mg) and compound BB-10 (100 mg, 248.87 μmol) were dissolved in 1,4-dioxane (10 mL) in a dry vial, and cesium carbonate (243.26 mg, 746.61 μmol) was added. The resulting mixture was stirred for 15 minutes. Then, the pH was measured to be greater than 9. Then, palladium acetate (11.17 mg, 49.77 μmol) and Xantphos (36.00 mg, 62.22 μmol) were added. The reaction solution was stirred at 100° C. under nitrogen for 4 hours. The reaction solution was filtered, and the filtrate was concentrated. The crude product was purified by a preparative thin layer chromatography silica gel plate (methanol/dichloromethane, the proportion of methanol was 10%) to give compound 18-2. MS m/z: 585.2 [M+1]⁺.

### Step 3: Synthesis of a Trifluoroacetate of Compound 18

Compound 18-2 (65 mg, 111.19 μmol) was dissolved in DCM (1 mL) in a dry vial, and trifluoroacetic acid (633.88 mg, 5.56 mmol, 411.61 μL) was added. The reaction solution was stirred at 20° C. for 2 hours. The reaction solution was directly concentrated to give a crude product. The crude product was purified by prep-HPLC (chromatography column: Waters Xbridge BEH C18 100*30 mm*10 μm; mobile phase: [water (NH₄HCO₃)-acetonitrile]; acetonitrile %: 1%-25%, 8 min), and 2 to 3 drops of trifluoroacetic acid were added to the fraction. The resulting product was concentrated to give a trifluoroacetate of compound 18. MS m/z: 485.3 [M+1]⁺; ¹H NMR (400 MHz, DMSO-d₆) δ 10.15 (s, 1H), 8.89 (s, 1H), 8.77 (d, J=8.4 Hz, 1H), 8.63-8.62 (m, 1H), 8.34-8.32 (m, 1H), 8.18 (s, 1H), 7.86-7.84 (m, 1H), 7.78-7.76 (m, 1H), 7.55 (d, J=8.4 Hz, 1H), 7.32-7.30 (m, 1H), 6.98-6.96 (m, 1H), 5.03-4.87 (m, 1H), 4.40-4.35 (m, 4H), 4.15-3.86 (m, 3H), 3.84-3.78 (m, 1H), 3.73-3.65 (m, 1H).

### Example 20

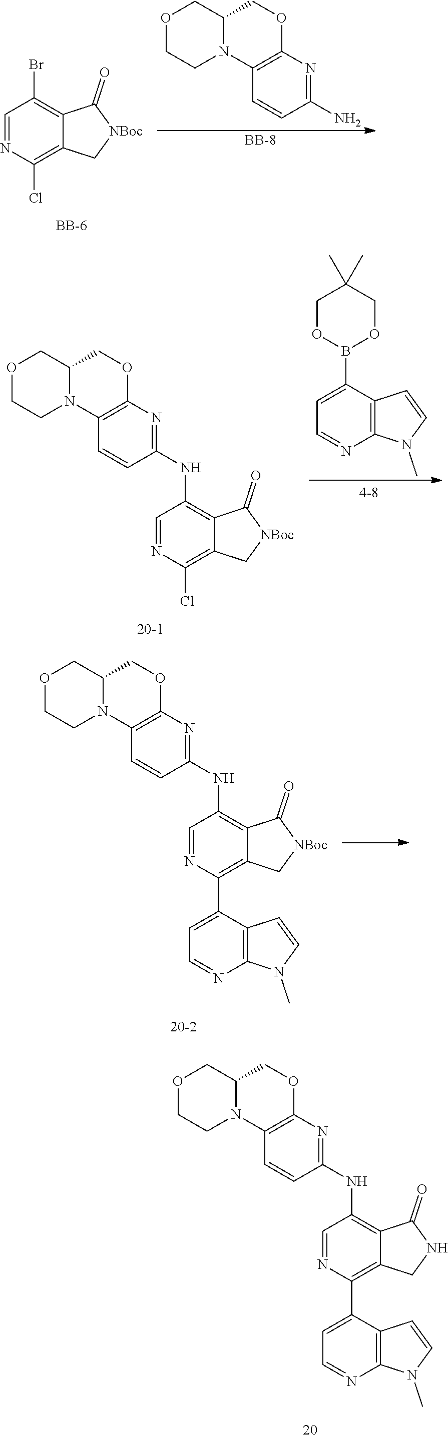

### Step 1: Synthesis of Compound 20-1

Compound BB-6 (300 mg, 863.08 μmol) was dissolved in 1,4-dioxane (6 mL) under nitrogen. Compound BB-8 (196.74 mg, 949.39 μmol), cesium carbonate (843.63 mg, 2.59 mmol), Xantphos (74.91 mg, 129.46 μmol), and palladium acetate (19.38 mg, 86.31 μmol) were added. The resulting mixture was reacted at 110° C. for 2 hours. The mixture was cooled and vacuum filtered. The residue was washed with dichloromethane (20 mL). The filtrate was concentrated under reduced pressure. The resulting crude product was purified by column chromatography (ethyl acetate/petroleum ether=0%-35%) to give compound 20-1. MS m/z: 474.1 [M+1]⁺; ¹H NMR (400 MHz, DMSO-d₆) δ 9.49 (s, 1H), 8.92 (s, 1H), 7.30 (d, J=8.4 Hz, 1H), 6.78 (d, J=8.4 Hz, 1H), 4.73 (s, 2H), 4.39-4.37 (m, 1H), 4.07-4.03 (m, 1H), 3.99-3.94 (m, 1H), 3.91-3.89 (m, 1H), 3.65-3.54 (m, 2H), 3.22-3.14 (m, 1H), 3.10-3.02 (m, 1H), 2.69-2.62 (m, 1H), 1.54 (s, 9H).

### Step 2: Synthesis of Compound 20-2

Compound 20-1 (142 mg, 299.64 μmol) was dissolved in 1,4-dioxane (5 mL) and water (1 mL) under nitrogen. Compound 4-8 (109.71 mg, 449.45 μmol), potassium phosphate (127.21 mg, 599.27 μmol), and RuPhos Pd G2 (23.58 mg, 29.96 μmol) were added. The resulting mixture was reacted at 100° C. for 2 hours. The mixture was cooled, and the system was concentrated under reduced pressure. The resulting crude product was purified by column chromatography (ethyl acetate/petroleum ether=0%-100%) to give compound 20-2. MS m/z: 570.2 [M+1]⁺; ¹H NMR (400 MHz, CDCl₃) δ 10.12 (s, 1H), 9.30 (s, 1H), 8.43 (d, J=5.2 Hz, 1H), 7.18 (d, J=4.8 Hz, 1H), 7.08 (d, J=8.4 Hz, 1H), 6.88 (d, J=3.6 Hz, 1H), 6.57 (d, J=8.4 Hz, 1H), 4.38-4.35 (m, 1H), 4.21-4.12 (m, 1H), 4.11-4.07 (m, 1H), 3.99-3.91 (m, 4H), 3.80-3.74 (m, 1H), 3.53 (s, 2H), 3.45-3.42 (m, 1H), 3.36-3.28 (m, 1H), 3.25-3.17 (m, 1H), 2.89-2.82 (m, 1H), 1.62 (s, 9H).

### Step 3: Synthesis of a Trifluoroacetate of Compound 20

Compound 20-2 (158 mg, 277.38 μmol) was dissolved in DCM (6 mL) under nitrogen, and trifluoroacetic acid (4.62 g, 40.52 mmol, 3 mL) was added. The resulting mixture was reacted at 25° C. for 2 hours. The solvent was concentrated under reduced pressure to give a crude product. The crude product was purified by prep-HPLC (chromatography column: Phenomenex Luna 80*30 mm*3 μm; mobile phase: [water (TFA)-acetonitrile]; acetonitrile %: 5%-35%, 8 min) to give a trifluoroacetate of compound 20. MS m/z: 470.1 [M+1]⁺; ¹H NMR (400 MHz, DMSO-d₆) δ 9.85 (s, 1H), 9.45 (s, 1H), 9.16 (s, 1H), 8.35 (d, J=4.8 Hz, 1H), 7.57 (d, J=3.6 Hz, 1H), 7.38 (d, J=4.8 Hz, 1H), 7.31 (d, J=8.4 Hz, 1H), 6.92 (d, J=3.6 Hz, 1H), 6.69 (d, J=8.4 Hz, 1H), 4.71 (s, 2H), 4.41-4.39 (m, 1H), 4.11-4.01 (m, 1H), 4.01-3.95 (m, 1H), 3.93-3.89 (m, 1H), 3.87 (s, 3H), 3.66-3.54 (m, 2H), 3.26-3.15 (m, 1H), 3.12-3.02 (m, 1H), 2.71-2.64 (m, 1H).

### Example 21 and Example 22

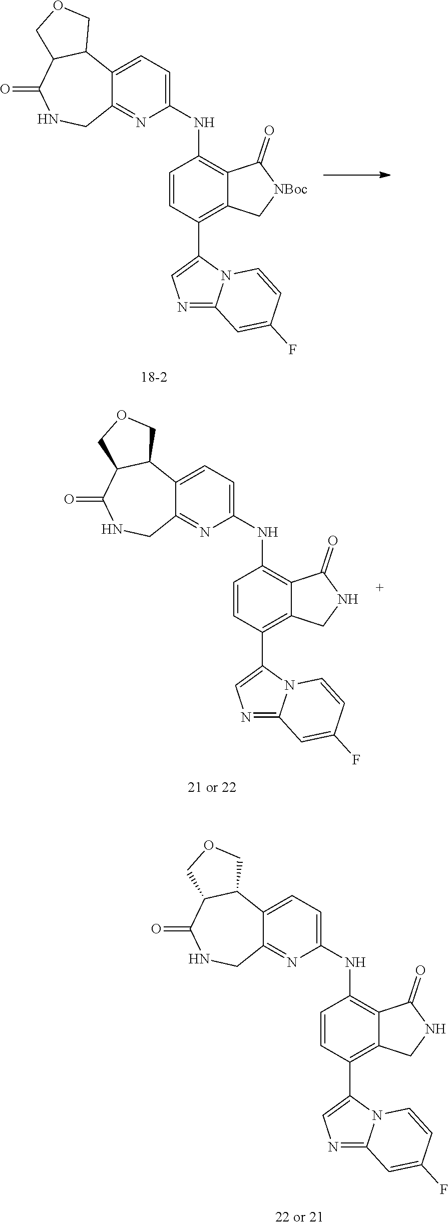 [SMILES: CC(C)(C)OC(=O)N1Cc2c(-c3cnc4cc(F)ccn34)ccc(Nc3ccc4c(n3)CNC(=O)C3COCC43)c2C1=O ; O=C1NCc2c(-c3cnc4cc(F)ccn34)ccc(Nc3ccc4c(n3)CNC(=O)[C@H]3COC[C@@H]43)c21 ; O=C1NCc2c(-c3cnc4cc(F)ccn34)ccc(Nc3ccc4c(n3)CNC(=O)[C@@H]3COC[C@H]43)c21]

### Step 1: Synthesis of a Trifluoroacetate of Compound 21 and a Trifluoroacetate of Compound 22

Compound 18-2 (100 mg, 171.06 μmol) was dissolved in DCM (1 mL) in a dry vial, and trifluoroacetic acid (975.20 mg, 8.55 mmol, 633.25 μL) was added. The reaction solution was stirred at 20° C. for 2 hours. The reaction solution was directly concentrated to give a crude product. The crude product was purified by a preparative thin layer chromatography silica gel plate (methanol/dichloromethane=16%). The resulting product was then separated and purified by SFC (chromatography column: REGIS (S, S) WHELK-01 (250 mm*25 mm, 10 m); mobile phase: A (carbon dioxide), B (ethanol, 0.1% ammonia), B %: 62%-62%, 20 min). The resulting solution was concentrated, and then two drops of trifluoroacetic acid were added dropwise to give a trifluoroacetate of compound 21 and a trifluoroacetate of compound 22.

The trifluoroacetate of compound 21: MS m/z: 485.3 [M+1]⁺; ¹H NMR (400 MHz, DMSO-d₆) δ ppm 10.14 (s, 1H), 8.89 (s, 1H), 8.77 (d, J=8.8 Hz, 1H), 8.61 (d, J=7.2 Hz, 1H), 8.34-8.32 (m, 1H), 8.16 (s, 1H), 7.84-7.82 (m, 1H), 7.78-7.76 (m, 1H), 7.56-7.54 (m, 1H), 7.32-7.30 (m, 1H), 6.99-6.97 (m, 1H), 4.91-4.87 (m, 1H), 4.40-4.36 (m, 4H), 3.92-3.88 (m, 2H), 3.84-3.68 (m, 3H). ee %=100%, retention time: 3.070 min.

The trifluoroacetate of compound 22: MS m/z: 485.3 [M+1]⁺; ¹H NMR (400 MHz, DMSO-d₆) δ ppm 10.14 (s, 1H), 8.89 (s, 1H), 8.77 (d, J=8.8 Hz, 1H), 8.63 (d, J=6.8 Hz, 1H), 8.33-8.31 (m, 1H), 8.20 (s, 1H), 7.88-7.86 (m, 1H), 7.79-7.77 (m, 1H), 7.56-7.54 (m, 1H), 7.34-7.32 (m, 1H), 6.99-6.97 (m, 1H), 4.91-4.87 (m, 1H), 4.41-4.36 (m, 4H), 3.92-3.85 (m, 2H), 3.81-3.53 (m, 2H), 3.38-3.34 (m, 1H). ee %=97.12%, SFC retention time: 5.061 min.

The analysis method of ee % detection: chromatography column: (S,S)-WHELK-01, 50×4.6 mm I.D., 3.5 μm; mobile phase: A: carbon dioxide B: ethanol (0.1% isopropylamine, v/v); B %=50-50%; flow rate: 4 mL/min; pressure: 1800 psi.

### Example 23

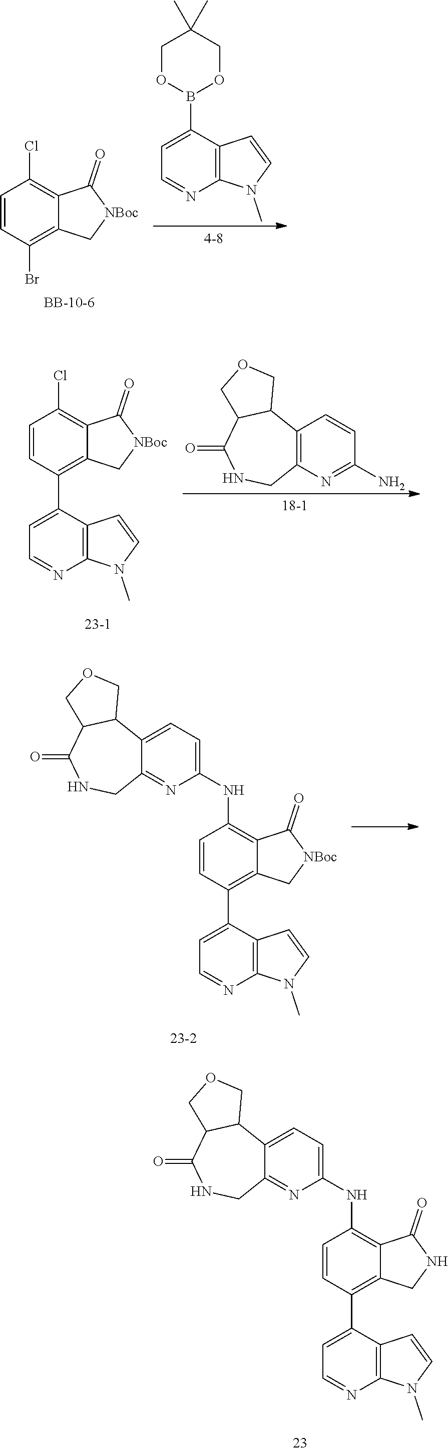

### Step 1: Synthesis of Compound 23-1

Compound BB-10-6 (1 g, 2.89 mmol) was dissolved in dioxane (20 mL) and water (4 mL) under nitrogen. Compound 4-8 (1.06 g, 4.33 mmol), cesium carbonate (2.82 g, 8.66 mmol), and Pd(dppf)Cl₂ (316.66 mg, 432.77 μmol) were added. The resulting mixture was reacted at 70° C. for 2 hours. The mixture was cooled, concentrated under reduced pressure to give a crude product. The crude product was purified by column chromatography (ethyl acetate/petroleum ether=0%-50%) to give compound 23-1. MS m/z: 398.1 [M+1]⁺; ¹H NMR (400 MHz, CDCl₃) δ 8.44 (d, J=4.8 Hz, 1H), 7.68 (d, J=8.0 Hz, 1H), 7.58 (d, J=8.0 Hz, 1H), 7.28 (s, 1H), 7.05 (d, J=4.8 Hz, 1H), 6.28 (d, J=3.6 Hz, 1H), 4.65 (s, 2H), 3.98 (s, 3H), 1.57 (s, 9H).

### Step 2: Synthesis of Compound 23-2

Compound 23-1 (45 mg, 113.11 μmol) was dissolved in dioxane (2 mL) under nitrogen. Compound 18-1 (27.28 mg, 124.42 μmol), cesium carbonate (110.56 mg, 339.32 mol), Xantphos (13.09 mg, 22.62 μmol), and palladium acetate (2.54 mg, 11.31 μmol) were added. The resulting mixture was reacted at 110° C. for 2 hours. The mixture was cooled, and the solvent was concentrated under reduced pressure. The resulting crude product was purified by column chromatography (ethyl acetate/petroleum ether=0%-100%) to give compound 23-2. MS m/z: 581.2 [M+1]⁺; ¹H NMR (400 MHz, DMSO-d₆) δ 9.87 (s, 1H), 8.81 (d, J=8.8 Hz, 1H), 8.38-8.26 (m, 2H), 7.81 (d, J=8.4 Hz, 1H), 7.62-7.53 (m, 2H), 7.25 (d, J=5.2 Hz, 1H), 7.08 (d, J=8.4 Hz, 1H), 6.47 (d, J=3.6 Hz, 1H), 4.92-4.83 (m, 1H), 4.80 (s, 2H), 4.43-4.39 (m, 1H), 4.38-4.35 (m, 1H), 3.97-3.86 (m, 5H), 3.86-3.68 (m, 2H), 3.38-3.34 (m, 1H), 1.50 (s, 9H).

### Step 3: Synthesis of a Trifluoroacetate of Compound 23

Compound 23-2 (69 mg, 118.84 μmol) was dissolved in DCM (2 mL) under nitrogen, and trifluoroacetic acid (0.5 mL) was added. The resulting mixture was reacted at 25° C. for 2 hours. The solvent was concentrated under reduced pressure to give a crude product. The crude product was added to methanol (10 mL), and the resulting mixture was reacted at 25° C. for 0.5 hours. The mixture was vacuum filtered, and the residue was washed with methanol (5 mL). The residue was taken and added to water (10 mL). 3 drops of trifluoroacetic acid were added, and the mixture was mixed well to give a trifluoroacetate of compound 23. MS m/z: 481.1 [M+1]⁺; ¹H NMR (400 MHz, DMSO-d₆) δ 10.17 (s, 1H), 8.84 (s, 1H), 8.73 (d, J=8.4 Hz, 1H), 8.35-8.26 (m, 2H), 7.75 (d, J=8.4 Hz, 1H), 7.59 (d, J=3.2 Hz, 1H), 7.53 (d, J=8.4 Hz, 1H), 7.27 (d, J=4.8 Hz, 1H), 6.95 (d, J=8.4 Hz, 1H), 6.47 (d, J=3.2 Hz, 1H), 4.90-4.84 (m, 1H), 4.46 (s, 2H), 4.42-4.33 (m, 2H), 3.98-3.86 (m, 5H), 3.84-3.78 (m, 1H), 3.73-3.69 (m, 1H), 3.35-3.33 (m, 1H).

### Example 24 and Example 40

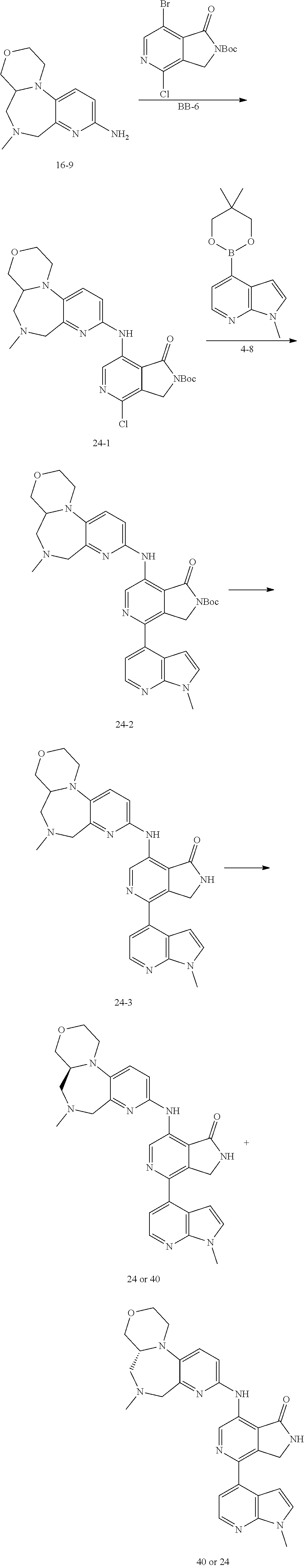

### Step 1: Synthesis of Compound 24-1

Compound 16-9 (145 mg, 618.87 μmol) was dissolved in dioxane (4 mL) under nitrogen. Compound BB-6 (236.63 mg, 680.76 μmol), cesium carbonate (604.92 mg, 1.86 mmol), Xantphos (71.62 mg, 123.77 μmol), and palladium acetate (13.89 mg, 61.89 μmol) were added. The resulting mixture was reacted at 110° C. for 2 hours. The mixture was cooled, and the solvent was concentrated under reduced pressure. The resulting crude product was purified by column chromatography (ethyl acetate/petroleum ether=0%-100%) to give compound 24-1. MS m/z: 501.1 [M+1]⁺.

### Step 2: Synthesis of Compound 24-2

Compound 24-1 (74 mg, 147.71 μmol) was dissolved in dioxane (4 mL) and water (0.8 mL) under nitrogen. Compound 4-8 (54.08 mg, 221.57 μmol), potassium phosphate (94.06 mg, 443.13 μmol), and XPhos Pd G2 (11.62 mg, 14.77 μmol) were added. The resulting mixture was reacted at 100° C. for 2 hours. The mixture was cooled, and the system was concentrated under reduced pressure. The resulting crude product was purified by column chromatography (methanol/ethyl acetate=0%-10%) to give compound 24-2. MS m/z: 597.2 [M+1]⁺.

### Step 3: Synthesis of a Trifluoroacetate of Compound 24-3

Compound 24-2 (36 mg, 60.33 μmol) was dissolved in DCM (2.5 mL) under nitrogen, and trifluoroacetic acid (0.5 mL) was added. The resulting mixture was reacted at 25° C. for 1 hour. The solvent was concentrated under reduced pressure to give a crude product. The crude product was purified by prep-HPLC (chromatography column: Phenomenex Luna C18 75*30 mm*3 μm; mobile phase [water (TFA)-acetonitrile]; acetonitrile %: 1%-30%, 8 min) to give a trifluoroacetate of compound 24-3. MS m/z: 497.2 [M+1]⁺; ¹H NMR (400 MHz, CD₃OD) δ 10.12 (s, 1H), 8.40 (d, J=5.2 Hz, 1H), 7.63 (d, J=8.8 Hz, 1H), 7.53-7.47 (m, 1H), 7.40 (d, J=5.2 Hz, 1H), 7.14 (d, J=8.8 Hz, 1H), 6.79 (d, J=3.6 Hz, 1H), 4.80-4.70 (m, 2H), 4.67 (s, 2H), 3.95-3.82 (m, 6H), 3.74-3.64 (m, 2H), 3.50-3.35 (m, 4H), 3.10 (s, 3H).

### Step 4: Synthesis of Compound 24, a Trifluoroacetate of Compound 24, Compound 40 and a Trifluoroacetate of Compound 40

The trifluoroacetate of compound 24-3 was subjected to SFC chiral separation (chromatography column: DAICEL CHIRALPAK IC (250 mm*25 mm, 10 m); mobile phase: A n-heptane, B isopropanol/acetonitrile=2/1, 0.1% ammonia, B %: 40%) to give compound 24 and compound 40.

Compound 24: ¹H NMR (DMSO-d₆, 400 MHz) δ 9.98 (s, 1H), 9.60 (s, 1H), 9.18 (s, 1H), 8.35 (d, 1H, J=3.6 Hz), 7.57 (d, 1H, J=3.6 Hz), 7.45 (d, 1H, J=8.8 Hz), 7.38 (d, 1H, J=5.2 Hz), 7.00 (d, 1H, J=8.4 Hz), 6.91 (d, 1H, J=3.2 Hz), 4.71 (s, 2H), 3.91-4.09 (m, 2H), 3.88 (s, 3H), 3.75-3.77 (m, 3H), 3.50-3.68 (m, 1H), 3.17-3.31 (m, 5H), 2.68-3.10 (m, 3H); ee %=97.35%; retention time: 3.147 min.

Compound 40: ¹H NMR (DMSO-d₆, 400 MHz) δ 9.99 (s, 1H), 9.64 (s, 1H), 9.20 (s, 1H), 8.35 (d, 1H, J=3.6 Hz), 7.58 (d, 1H, J=3.5 Hz), 7.45 (d, 1H, J=5.2 Hz), 7.38 (d, 1H, J=5.2 Hz), 7.07 (d, 1H, J=8.8 Hz), 6.91 (d, 1H, J=3.2 Hz), 4.72 (s, 2H), 3.91-4.10 (m, 2H), 3.88 (s, 3H), 3.70-3.80 (m, 3H), 3.51-3.62 (m, 1H), 3.17-3.30 (m, 5H), 2.66-3.10 (m, 3H); ee %=97.65%; retention time: 3.683 min.

The analysis method of ee % detection: (chromatography column: DAICEL CHIRALPAK IC (50*4.6 mm 3 m); mobile phase: A n-heptane (0.1% DEA), B isopropanol/acetonitrile=2/1, B %: 45%).

Compound 24 (658 mg, 1.33 mmol) was dissolved in DCM (7 mL), and TFA (492.61 μL) was added. The resulting mixture was stirred at 25° C. for 1 hour. The mixture was directly concentrated under reduced pressure to give a trifluoroacetate of compound 24. MS m/z: 497.1 [M+1]⁺; ee %=97.85%; ¹H NMR (DMSO-d₆, 400 MHz) δ 10.03 (s, 1H), 9.72 (s, 1H), 9.24 (s, 1H), 8.35 (d, 1H, J=5.2 Hz), 7.60 (m, 2H), 7.41 (d, 1H, J=5.2 Hz), 7.17-7.24 (m, 1H), 6.92 (d, 1H, J=3.6 Hz), 4.60 (s, 2H), 3.88 (s, 3H), 3.36-3.78 (m, 11H), 3.01 (s, 3H).

Compound 40 (1.26 g, 2.54 mmol) was dissolved in DCM (13 mL), and TFA (942.43 μL) was added. The resulting mixture was stirred at 25° C. for 1 hour. The mixture was directly concentrated under reduced pressure to give a trifluoroacetate of compound 40. MS m/z: 497.1 [M+1]⁺; ee %=97.84%; ¹H NMR (DMSO-d₆, 400 MHz) δ 10.00 (s, 1H), 9.72 (s, 1H), 9.25 (s, 1H), 8.37 (d, 1H, J=5.2 Hz), 7.59 (m, 2H), 7.41 (d, 1H, J=5.2 Hz), 7.21 (m, 1H), 6.92 (d, 1H, J=3.6 Hz), 4.73 (s, 2H), 4.65-4.62 (m, 1H), 3.88 (s, 3H), 3.78-3.71 (m, 5H), 3.71-3.35 (m 5H), 3.00 (s, 3H).

### Example 25

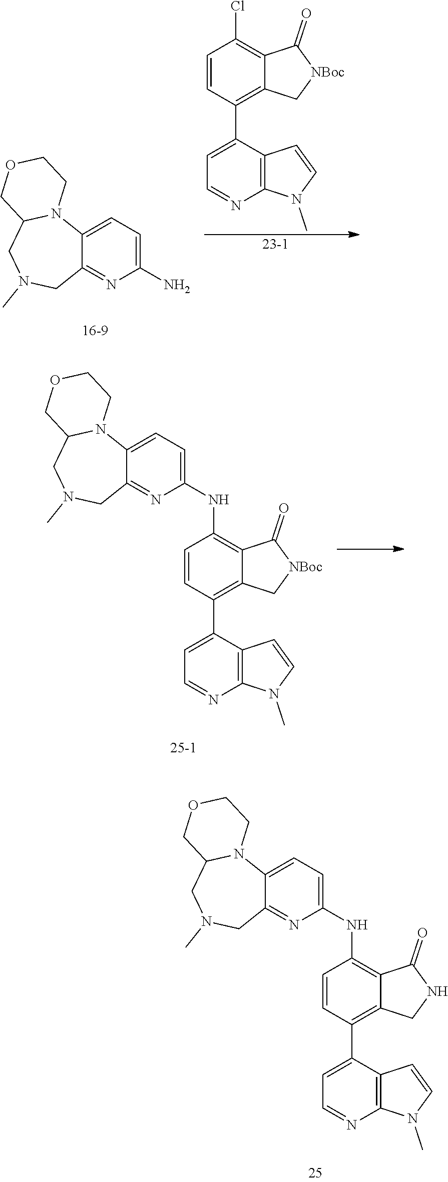

### Step 1: Synthesis of Compound 25-1

Compound 16-9 (40 mg, 170.72 μmol) was dissolved in dioxane (2 mL) under nitrogen. Compound 23-1 (67.92 mg, 170.72 μmol), potassium carbonate (70.79 mg, 512.17 mol), Ruphos (15.93 mg, 34.14 μmol), and Pd₂(dba)₃ (15.63 mg, 17.07 μmol) were added. The resulting mixture was reacted at 110° C. for 2 hours. The mixture was cooled, and the solvent was concentrated under reduced pressure. The resulting crude product was purified by column chromatography (methanol/ethyl acetate=0%-10%) to give compound 25-1. MS m/z: 596.2 [M+1]⁺.

### Step 2: Synthesis of a Trifluoroacetate of Compound 25

Compound 25-1 (82 mg, 137.66 μmol) was dissolved in DCM (3 mL) under nitrogen, and trifluoroacetic acid (0.5 mL) was added. The resulting mixture was reacted at 25° C. for 16 hours. The solvent was concentrated under reduced pressure to give a crude product. The crude product was purified by prep-HPLC (chromatography column: Phenomenex C18 80*30 mm*3 μm; mobile phase: [water (TFA)-acetonitrile]; acetonitrile %: 1%-30%, 8 min) to give a trifluoroacetate of compound 25. MS m/z: 496.1 [M+1]⁺; ¹H NMR (400 MHz, CD₃OD) δ 8.76 (d, J=8.4 Hz, 1H), 8.35 (d, J=5.2 Hz, 1H), 7.77 (d, J=8.4 Hz, 1H), 7.59 (d, J=8.8 Hz, 1H), 7.49 (d, J=3.6 Hz, 1H), 7.30 (d, J=5.2 Hz, 1H), 7.08 (d, J=8.8 Hz, 1H), 6.53 (d, J=3.2 Hz, 1H), 4.85-4.60 (m, 3H), 4.49 (s, 2H), 3.95 (s, 3H), 3.93-3.82 (m, 3H), 3.76-3.60 (m, 2H), 3.51-3.34 (m, 3H), 3.08 (s, 3H).

### Example 26

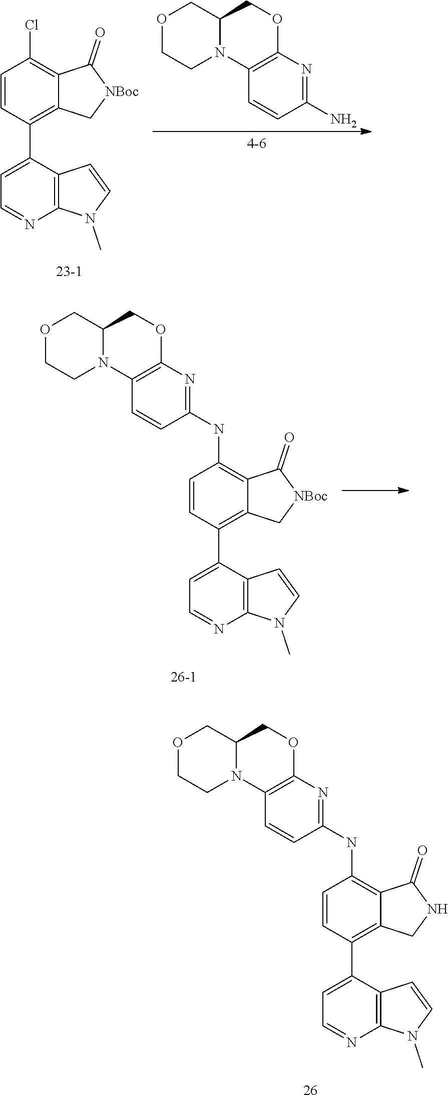

### Step 1: Synthesis of Compound 26-1

Compound 23-1 (0.1 g, 251.35 μmol) and 4-6 (52.09 mg, 251.35 μmol) were added to anhydrous dioxane (2 mL). Then, potassium carbonate (104.21 mg, 754.05 μmol), Ruphos (17.59 mg, 37.70 μmol), and Pd₂(dba)₃ (23.02 mg, 25.13 μmol) were added. The resulting mixture was reacted at 110° C. for 2 hours. The reaction solution was added to water (30 mL), and the resulting mixture was extracted with ethyl acetate (30 mL×3). The organic phases were combined, dried over anhydrous sodium sulfate, and filtered. The filtrate was concentrated under reduced pressure to give compound 26-1. MS m/z: 569.2 [M+1]⁺.

### Step 2: Synthesis of a Trifluoroacetate of Compound 26

Compound 26-1 (0.12 g, 211.04 μmol) was added to DCM (2 mL), and then trifluoroacetic acid (1 mL) was added. The resulting mixture was reacted at 25° C. for 2 hours. The reaction solution was concentrated under reduced pressure, and the crude product was purified by prep-HPLC (chromatography column: Phenomenex Luna C18 75*30 mm*3 μm; mobile phase: [water (TFA)-acetonitrile]; acetonitrile %: 10%-40%, 8 min) to give a trifluoroacetate of compound 26. MS m/z: 469.1 [M+1]⁺; ¹H NMR (400 MHz, DMSO-d₆) δ ppm 9.85 (s, 1H), 8.74 (s, 1H), 8.47 (d, J=8.6 Hz, 1H), 8.31 (d, J=5.0 Hz, 1H), 7.71 (d, J=8.6 Hz, 1H), 7.58 (s, 1H), 7.30-7.26 (m, 2H), 6.60 (d, J=8.4 Hz, 1H), 6.47 (s, 1H), 4.45 (s, 2H), 4.39-4.36 (m, 1H), 4.06-4.01 (m, 5H), 3.92-3.87 (m, 3H), 3.20-3.17 (m, 1H), 3.04 (s, 1H), 2.68-2.65 (m, 1H).

### Example 27

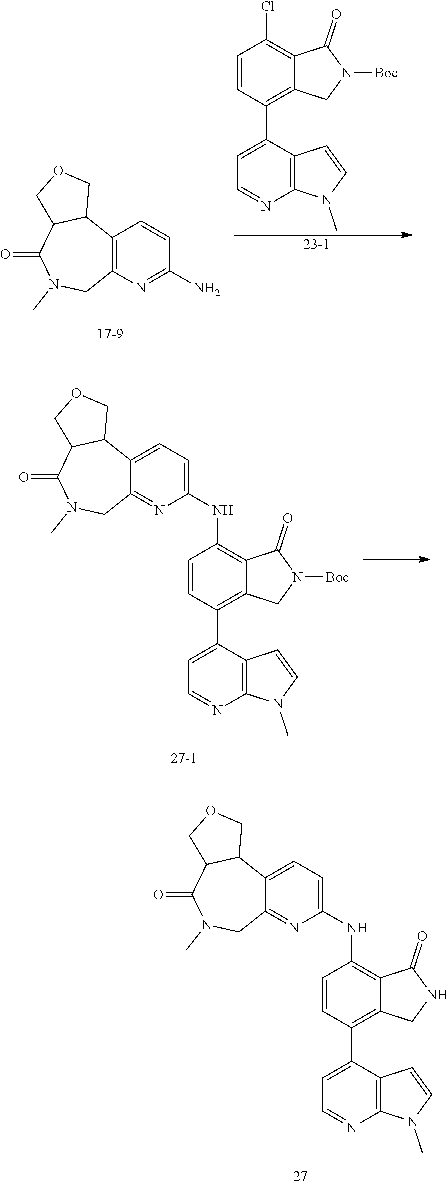

### Step 1: Synthesis of Compound 27-1

Compound 17-9 (40 mg, 171.48 μmol) and compound 23-1 (68.22 mg, 171.48 mol) were dissolved in dioxane (8 mL) in a dry vial, and cesium carbonate (167.62 mg, 514.44 mol) was added. The resulting mixture was stirred for 15 minutes. Then, the pH was measured to be greater than 9. Then, palladium acetate (7.7 mg, 34.3 μmol) and Xantphos (12.40 mg, 21.43 mol) were added. The reaction solution was stirred at 110° C. under nitrogen for 1 hour. The reaction solution was filtered, and the filtrate was concentrated. The crude product was separated by column chromatography (ethyl acetate/petroleum ether=0%-10%) to give compound 27-1.

### Step 2: Synthesis of a Trifluoroacetate of Compound 27

Compound 27-1 (70 mg, 117.71 μmol) was dissolved in DCM (1 mL) in a dry vial, and trifluoroacetic acid (671.09 mg, 5.89 mmol, 435.77 μL) was added. The reaction solution was stirred at 25° C. for 2 hours. The reaction solution was concentrated to give a crude product. The crude product was purified by prep-HPLC (chromatography column: Phenomenex luna C18 100*40 mm*3 μm; mobile phase: [water (TFA)-acetonitrile]; acetonitrile %: 10%-48%, 8 min) to give a trifluoroacetate of compound 27. MS m/z: 495.2 [M+1]⁺; ¹H NMR (400 MHz, DMSO-d₆) δ 10.167 (s, 1H), 8.84 (s, 1H), 8.71 (d, J=8.4 Hz, 1H), 8.12 (d, J=4.8 Hz, 1H), 7.77 (d, J=8.4 Hz, 1H), 7.59 (s, 1H), 7.50 (d, J=8.0 Hz, 1H), 7.28 (d, J=5.2 Hz, 1H), 6.98 (d, J=8.4 Hz, 1H), 6.49 (s, 1H), 5.30 (d, J=15.2 Hz, 1H), 4.46-4.43 (m, 4H), 4.03-3.99 (m, 1H), 3.97-3.92 (m, 2H), 3.90 (s, 3H), 3.72-3.67 (m, 1H), 3.30-3.27 (m, 1H), 2.94 (s, 3H).

### Example 28

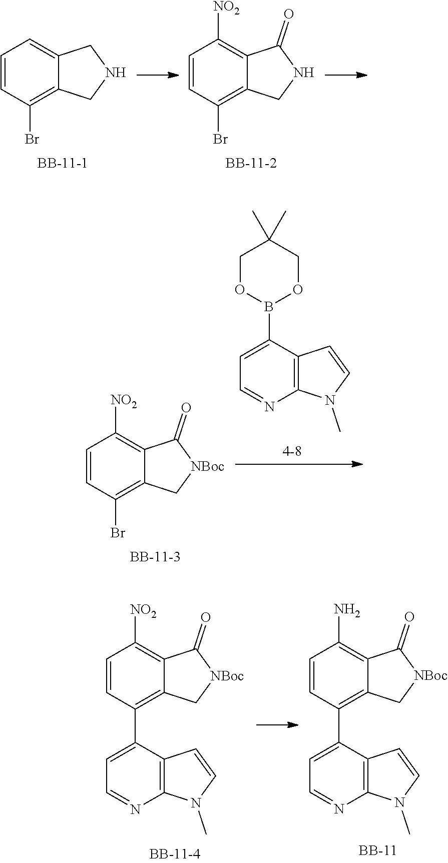

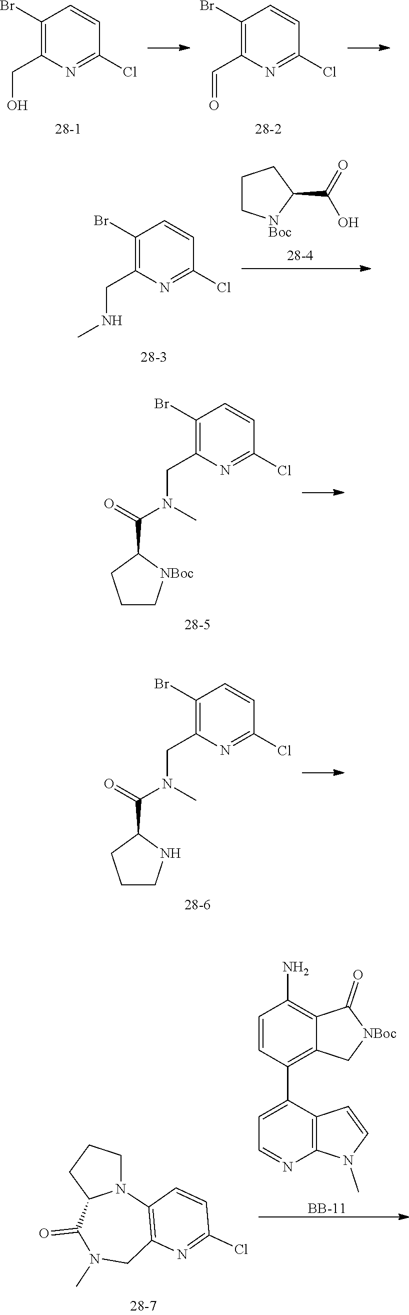

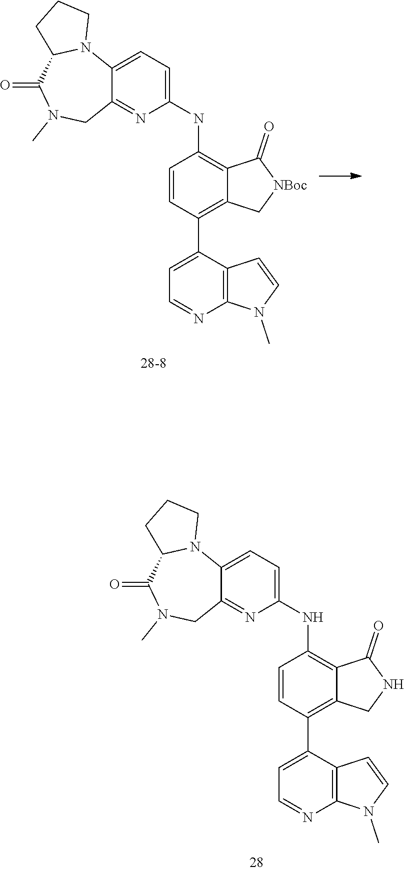 [SMILES: CN1Cc2nc(Nc3ccc(-c4ccnc5c4ccn5C)c4c3C(=O)N(C(=O)OC(C)(C)C)C4)ccc2N2CCC[C@H]2C1=O ; CN1Cc2nc(Nc3ccc(-c4ccnc5c4ccn5C)c4c3C(=O)NC4)ccc2N2CCC[C@H]2C1=O]

### Step 1: Synthesis of Compound BB-11-2

Compound BB-11-1 (40 g, 188.64 mmol) was added to concentrated sulfuric acid (2000 mL) under nitrogen, and the resulting mixture was cooled to −10° C. Concentrated nitric acid (21.55 mL) was slowly added dropwise, and the mixture was reacted at −10° C. for 2 hours. The mixture was warmed to 20° C. and reacted for 2 hours. The reaction solution was slowly poured into ice water (2000 mL) and filtered. The filter cake was washed with water (200 mL). The filter cake was dried to give a crude product, which was added to ethanol (400 mL). The resulting mixture was stirred for 2 hours, and then filtered. The filter cake was concentrated under reduced pressure to give compound BB-11-2. ¹H NMR (400 MHz, CDCl₃) δ 7.87 (d, J=8.4 Hz, 1H), 7.74 (d, J=8.4 Hz, 1H), 7.44 (s, 1H), 4.47 (s, 2H).

### Step 2: Synthesis of Compound BB-11-3

Compound BB-11-2 (51 g, 198.41 mmol) was added to dioxane (510 mL) under nitrogen. Di-tert-butyl dicarbonate (64.95 g, 297.62 mmol, 68.37 mL), 4-dimethylaminopyridine (2.42 g, 19.84 mmol), and triethylamine (60.23 g, 595.24 mmol, 82.85 mL) were added. The resulting mixture was reacted at 20° C. for 12 hours. The reaction solution was concentrated under reduced pressure to give a crude product, which was added to methyl tert-butyl ether (250 mL). The resulting mixture was stirred for 2 hours and filtered. The filter cake was dried to give compound BB-11-3. ¹H NMR (400 MHz, CDCl₃) δ 7.84 (d, J=8.4 Hz, 1H), 7.60 (d, J=8.4 Hz, 1H), 4.65 (s, 2H), 1.49 (s, 9H).

### Step 3: Synthesis of Compound BB-11-4

Compound BB-11-3 (15 g, 42.00 mmol) and compound 4-8 (10.25 g, 42.00 mmol) were added to a mixed solution of dioxane (150 mL) and water (30 mL) under nitrogen. Potassium phosphate (17.83 g, 84.00 mmol) and XPhos Pd G2 (4.96 g, 6.30 mmol) were added. The resulting mixture was heated to 70° C. and reacted for 2 hours. The reaction solution was added to water (200 mL), and the resulting mixture was extracted with ethyl acetate (200 mL×3). The organic phases were combined, dried over anhydrous sodium sulfate, and filtered. The filtrate was concentrated under reduced pressure to give a crude product. The crude product was separated by column chromatography (ethyl acetate/petroleum ether=0%-20%) to give compound BB-11-4. MS m/z: 409.0 [M+1]⁺; ¹H NMR (400 MHz, CDCl₃) δ 8.39 (d, J=5.0 Hz, 1H), 7.80 (d, J=2.0 Hz, 2H), 7.24-7.19 (m, 1H), 6.99 (d, J=5.0 Hz, 1H), 6.19 (d, J=3.6 Hz, 1H), 4.64 (s, 2H), 3.91 (s, 3H), 1.48 (s, 9H).

### Step 4: Synthesis of Compound BB-11

Compound BB-11-4 (11.7 g, 28.65 mmol) was added to a mixed solution of ethanol (220 mL) and water (44 mL) under nitrogen. Iron powder (8.00 g, 143.24 mmol) and ammonium chloride (7.66 g, 143.24 mmol) were added. The resulting mixture was heated to 60° C. and reacted for 2 hours. The mixture was cooled and filtered. The filter cake was washed with a mixture of dichloromethane and methanol (10:1, 200 mL), and the filtrate was concentrated under reduced pressure. Then, water (100 mL) was added, and the resulting mixture was extracted with dichloromethane (100 mL×3). The organic phases were combined, dried over anhydrous sodium sulfate, and filtered. The filtrate was concentrated under reduced pressure to give compound BB-11. MS m/z: 379.0 [M+1]⁺.

### Step 5: Synthesis of Compound 28-2

Compound 28-1 (5 g, 22.48 mmol) was dissolved in DCM (50 mL) under nitrogen, and Dess-Martin periodinane (11.44 g, 26.97 mmol, 8.35 mL) was added. The resulting mixture was reacted at 25° C. for 2 hours. A saturated sodium carbonate solution (50 mL) was added, and the aqueous phase was extracted with dichloromethane (50 mL×3). The layers were separated. The organic phase was washed with saturated brine (20 mL), dried over anhydrous sodium sulfate, and filtered. The organic phase was concentrated under reduced pressure to give a crude product. The crude product was purified by column chromatography (ethyl acetate/petroleum ether=0%-50%) to give compound 28-2. MS m/z: 222.0, 220.0 [M+1]⁺; ¹H NMR (400 MHz, CDCl₃) δ 10.11 (s, 1H), 7.99 (d, J=8.4 Hz, 1H), 7.41 (d, J=8.4 Hz, 1H).

### Step 6: Synthesis of Compound 28-3

Methanol (21 mL), compound 28-2 (420 mg, 1.91 mmol), and methylamine hydrochloride (257.27 mg, 3.81 mmol) were added to a thumb flask. The resulting mixture was started to be stirred, and then potassium acetate (392.66 mg, 4.00 mmol) was added. The resulting mixture was stirred at 25° C. for 2 hours. Then, NaBH(OAc)₃ (1.21 g, 5.72 mmol) was added, and the mixture was stirred at 25° C. for another 1 hour. The reaction solution was poured into a saturated sodium carbonate solution (50 mL), and the aqueous phase was extracted with ethyl acetate (20 mL×3). The organic phases were combined, dried over anhydrous sodium sulfate, filtered, and concentrated. The crude product was purified by column chromatography (dichloromethane:methanol=2%-10%) to give compound 28-3. MS m/z: 234.8, 236.8 [M+1]⁺; ¹H NMR (400 MHz, DMSO-d₆) δ 8.10 (d, J=8.4 Hz, 1H), 7.39 (d, J=8.4 Hz, 1H), 3.84 (s, 2H), 2.34 (s, 3H).

### Step 7: Synthesis of Compound 28-5

Compound 28-3 (400 mg, 1.70 mmol) was dissolved in DMF (4 mL) under nitrogen. Compound 28-4 (470.26 mg, 2.18 mmol), HATU (968.70 mg, 2.55 mmol), and DIEA (658.52 mg, 5.10 mmol, 887.50 μL) were added. The resulting mixture was reacted at 25° C. for 0.5 hours. The reaction system was concentrated, and water (5 mL) was added. The aqueous phase was extracted with ethyl acetate (10 mL), and the layers were separated. The organic phase was dried over anhydrous sodium sulfate, filtered, and concentrated under reduced pressure to give a crude product. The crude product was purified by column chromatography (ethyl acetate/petroleum ether=30%-50%) to give compound 28-5. MS m/z: 432.2, 434.2 [M+1]⁺.

### Step 8: Synthesis of a Hydrochloride of Compound 28-6

Compound 28-5 (312 mg, 720.99 μmol) was added to hydrochloric acid/ethyl acetate (4 M, 5 mL), and the resulting mixture was reacted at 25° C. for 2 hours. The reaction system was concentrated to give a hydrochloride of compound 28-6. MS m/z: 332.0, 334.0 [M+1]⁺.

### Step 9: Synthesis of Compound 28-7

The hydrochloride of compound 28-6 (294 mg) was dissolved in dioxane (3 mL) under nitrogen. Cesium carbonate (1.04 g, 3.19 mmol) and Xantphos Pd G₄ (76.66 mg, 79.66 mol) were added. The resulting mixture was reacted at 110° C. for 2 hours. The reaction system was concentrated, and water (10 mL) was added. The aqueous phase was extracted with ethyl acetate (10 mL), and the layers were separated. The organic phase was dried over anhydrous sodium sulfate, filtered, and concentrated under reduced pressure to give a crude product. The crude product was purified by column chromatography (ethyl acetate/petroleum ether=30%-60%) to give compound 28-7. MS m/z: 252.1 [M+1]⁺; ¹H NMR (400 MHz, CDCl₃) δ 6.99 (d, J=8.6 Hz, 1H), 6.65 (d, J=8.6 Hz, 1H), 5.40 (d, J=16.8 Hz, 1H), 4.90 (m, 1H), 3.98 (d, J=17.0 Hz, 1H), 3.23-3.12 (m, 2H), 3.05 (s, 3H), 2.59 (m, 1H), 2.03-1.85 (m, 3H).

### Step 10: Synthesis of Compound 28-8

Compound BB-11 (50 mg, 132.13 μmol) was dissolved in dioxane (1 mL) under nitrogen. Compound 28-7 (39.91 mg, 158.55 μmol), cesium carbonate (86.10 mg, 264.25 μmol), palladium acetate (2.97 mg, 13.21 μmol), and BINAP (12.34 mg, 19.82 μmol) were added. The resulting mixture was reacted at 110° C. for 2 hours. The reaction system was concentrated, and water (10 mL) was added. The aqueous phase was extracted with ethyl acetate (10 mL), and the layers were separated. The organic phase was dried over anhydrous sodium sulfate, filtered, and concentrated under reduced pressure to give a crude product. The crude product was purified by a preparative thin layer chromatography silica gel plate (ethyl acetate/petroleum ether=30%) to give compound 28-8. MS m/z: 594.5 [M+1]⁺.

### Step 11: Synthesis of Compound 28

Compound 28-8 (30 mg, 50.53 μmol) was dissolved in DCM (1 mL) under nitrogen, and trifluoroacetic acid (1 mL) was added. The resulting mixture was reacted at 25° C. for 2 hours. The reaction system was concentrated under reduced pressure, and a saturated sodium bicarbonate solution (5 mL) was added. The aqueous phase was extracted with ethyl acetate (5 mL), and the layers were separated. The organic phase was dried over anhydrous sodium sulfate, filtered, and concentrated under reduced pressure to give a crude product. The crude product was purified by a preparative thin layer chromatography silica gel plate (ethyl acetate) to give compound 28. MS m/z: 494.2 [M+1]⁺; ¹H NMR (400 MHz, DMSO-d₆) δ 9.76 (s, 1H), 8.72 (s, 1H), 8.38 (d, J=8.6 Hz, 1H), 8.30 (d, J=5.0 Hz, 1H), 7.70 (d, J=8.6 Hz, 1H), 7.57 (d, J=3.6 Hz, 1H), 7.25 (d, J=5.0 Hz, 1H), 6.99 (d, J=8.8 Hz, 1H), 6.93 (d, J=8.6 Hz, 1H), 6.47 (d, J=3.6 Hz, 1H), 5.67 (d, J=16.9 Hz, 1H), 5.01-5.09 (m, 1H), 4.45 (s, 2H), 4.05 (d, J=17.0 Hz, 1H), 3.87 (s, 3H), 3.02 (s, 3H), 2.42 (m, 2H), 1.84-1.93 (m, 2H), 1.37-1.13 (m, 2H).

### Example 29

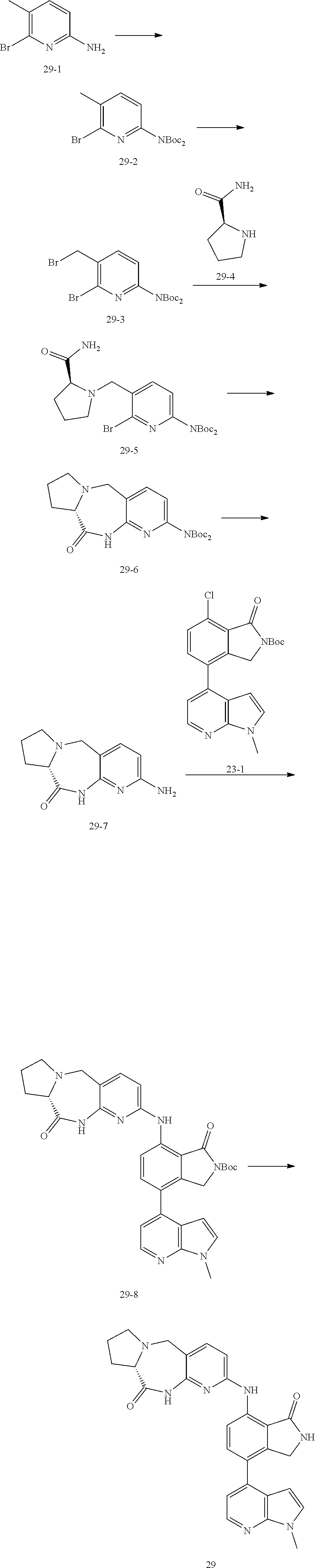

### Step 1: Synthesis of Compound 29-2

Compound 29-1 (5 g, 26.73 mmol) and di-tert-butyl dicarbonate (17.50 g, 80.20 mmol, 18.42 mL) were added to tetrahydrofuran (50 mL), and 4-dimethylaminopyridine (326.59 mg, 2.67 mmol) was added. The resulting mixture was reacted at 25° C. for 12 hours. The reaction solution was concentrated under reduced pressure to give a crude product. The crude product was purified by column chromatography (ethyl acetate/petroleum ether=0%-1%) to give compound 29-2. MS m/z: 386.9, 388.9 [M+1]⁺.

### Step 2: Synthesis of Compound 29-3

Compound 29-2 (2.5 g, 6.46 mmol) was added to 1,2-dichloroethane (25 mL). N-bromosuccinimide (1.72 g, 9.68 mmol) and azobisisobutyronitrile (106.01 mg, 645.55 μmol) were added. The resulting mixture was heated to 80° C. and reacted for 12 hours. The reaction solution was added to water (50 mL), and the mixture was extracted with dichloromethane (50 mL×2). The organic phases were combined, dried over anhydrous sodium sulfate, and filtered. The filtrate was concentrated under reduced pressure. The crude product was separated by column chromatography (ethyl acetate/petroleum ether=0%-2%) to give compound 29-3. MS m/z: 466.6, 468.7 [M+1]⁺.

### Step 3: Synthesis of Compound 29-5

Compound 29-3 (0.9 g, 1.93 mmol) and compound 29-4 (264.45 mg, 2.32 mmol) were added to acetonitrile (27 mL), and DIEA (748.57 mg, 5.79 mmol, 1.01 mL) was added. The resulting mixture was reacted at 25° C. for 12 hours. The reaction solution was concentrated under reduced pressure, and the crude product was separated by column chromatography (ethyl acetate/petroleum ether=0%-50%) to give compound 29-5. MS m/z: 499.0, 501.0 [M+1]⁺.

### Step 4: Synthesis of Compound 29-6

Compound 29-5 (0.85 g, 1.70 mmol) was added to dioxane (17 mL). Cesium carbonate (1.11 g, 3.40 mmol) and Xantphos Pd G₄ (163.80 mg, 170.21 μmol) were added. The resulting mixture was heated to 110° C. and reacted for 4 hours. Water (50 mL) was added to the reaction system, and the aqueous phase was extracted with ethyl acetate (50 mL×3). The layers were separated. The organic phase was dried over anhydrous sodium sulfate, filtered, and concentrated under reduced pressure to give a crude product. The crude product was separated by column chromatography (ethyl acetate/petroleum ether=0%-50%) to give compound 29-6. MS m/z: 419.1 [M+1]⁺.

### Step 5: Synthesis of Compound 29-7

Compound 29-6 (0.5 g, 1.19 mmol) was added to trifluoroacetic acid (2 mL) and DCM (4 mL). The resulting mixture was reacted at 25° C. for 2 hours. The reaction solution was concentrated under reduced pressure, and dissolved with water (10 mL) and dichloromethane (10 mL). Then, the mixture was added to a saturated aqueous solution of sodium carbonate (50 mL). The resulting mixture was extracted with a mixture of dichloromethane and methanol (10:1, 50 mL×5), and the layers were separated. The organic phase was dried over anhydrous sodium sulfate and filtered. The filtrate was concentrated under reduced pressure to give compound 29-7. MS m/z: 218.9 [M+1]⁺.

### Step 6: Synthesis of Compound 29-8

Compound 23-1 (60 mg, 150.81 μmol) and compound 29-7 (39.50 mg, 180.97 mol) were added to dioxane (2 mL). Potassium carbonate (41.69 mg, 301.62 μmol), Ruphos (14.07 mg, 30.16 μmol), and Pd₂(dba)₃ (13.81 mg, 15.08 μmol) were added. The resulting mixture was heated to 110° C. and reacted for 2 hours. Water (50 mL) was added to the reaction system, and the aqueous phase was extracted with ethyl acetate (50 mL×3). The layers were separated. The organic phase was dried over anhydrous sodium sulfate, filtered, and concentrated under reduced pressure to give compound 29-8. MS m/z: 580.2 [M+1]⁺.

### Step 7: Synthesis of a Trifluoroacetate of Compound 29

Compound 29-8 (60 mg, 103.51 μmol) was added to DCM (2 mL) and trifluoroacetic acid (1 mL). The resulting mixture was reacted at 25° C. for 2 hours. The reaction solution was concentrated under reduced pressure, and purified by prep-HPLC (chromatography column Phenomenex Luna 80*30 mm*3 μm; mobile phase: [water (TFA)-acetonitrile]; acetonitrile %: 1%-25%, 8 min) to give a trifluoroacetate of compound 29. MS m/z: 480.3 [M+1]⁺; ¹H NMR (400 MHz, DMSO-d₆) δ 11.17 (m, 1H), 10.45 (s, 1H), 8.92-8.89 (m, 2H), 8.34 (d, J=4.8 Hz, 1H), 7.80-7.72 (m, 2H), 7.60 (d, J=3.6 Hz, 1H), 7.28 (d, J=4.8 Hz, 1H), 6.89 (d, J=8.2 Hz, 1H), 6.45 (d, J=3.4 Hz, 1H), 4.60-4.57 (m, 1H), 4.50 (s, 3H), 4.20-4.08 (m, 2H), 3.72-3.70 (m, 1H), 3.67-3.65 (m, 2H), 3.28-3.27 (m, 1H), 2.17 (s, 1H), 2.16-2.13 (m, 1H), 1.94-1.90 (m, 2H).

### Example 30

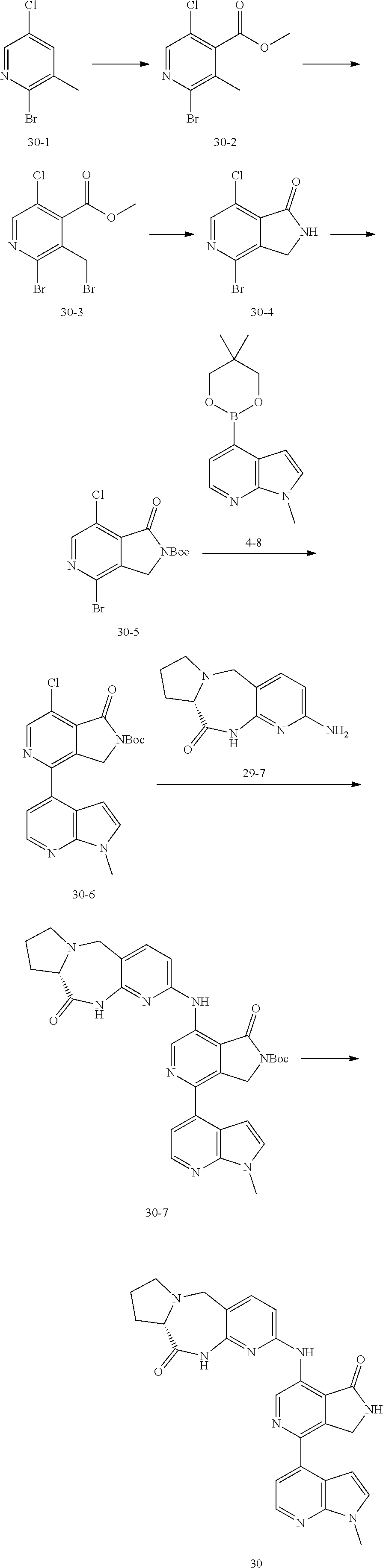

### Step 1: Synthesis of Compound 30-2

Compound 30-1 (75 g, 363.25 mmol) was dissolved in tetrahydrofuran (750 mL) under nitrogen, and the resulting mixture was cooled to −65° C. A solution of lithium diisopropylamide in tetrahydrofuran (2 M, 217.95 mL) was added. The resulting mixture was reacted at −65° C. for 1 hour, and dimethyl carbonate (65.44 g, 726.51 mmol, 61.16 mL) was added. The mixture was warmed to 20° C. and reacted for 3 hours. A saturated ammonium chloride solution (200 mL) was added to quench the reaction, and water (500 mL) was added. The mixture was extracted with ethyl acetate (1000 mL), dried over anhydrous sodium sulfate, filtered and concentrated to give a crude product. The crude product was purified by column chromatography (ethyl acetate/petroleum ether=1%-10%) to give compound 30-2. MS m/z: 264.0, 266.0 [M+1]⁺; ¹H NMR (400 MHz, CDCl₃) δ 8.20 (s, 1H), 3.93 (s, 3H), 2.04 (s, 3H).

### Step 2: Synthesis of Compound 30-3

Compound 30-2 (8.2 g, 31.00 mmol) was dissolved in 1,2-dichloroethane (80 mL) under nitrogen. N-bromosuccinimide (16.55 g, 93.00 mmol) and azobisisobutyronitrile (1.02 g, 6.20 mmol) were added. The resulting mixture was reacted at 80° C. for 5 hours. The mixture was cooled, and a saturated sodium sulfite solution (80 mL) was added to quench the reaction. The mixture was extracted with dichloromethane (50 mL×3). The organic phases were combined, washed with saturated sodium chloride solution (20 mL), dried over anhydrous sodium sulfate, and vacuum filtered. The filtrate was concentrated under reduced pressure to give compound 30-3. MS m/z: 343.8 [M+1]⁺.

### Step 3: Synthesis of Compound 30-4

Compound 30-3 (11.86 g, 34.54 mmol) was dissolved in ammonia/methanol solution (7M, 200 mL) under nitrogen. The resulting mixture was reacted at 25° C. for 16 hours and vacuum filtered. The residue was washed with methanol (20 mL), and the residue was collected to give compound 30-4. ¹H NMR (400 MHz, DMSO-d₆) δ 8.55 (s, 1H), 4.35 (s, 2H).

### Step 4: Synthesis of Compound 30-5

Compound 30-4 (2.94 g, 11.88 mmol) was dissolved in tetrahydrofuran (30 mL) under nitrogen. Di-tert-butyl dicarbonate (3.89 g, 17.82 mmol, 4.09 mL) and 4-dimethylaminopyridine (290.27 mg, 2.38 mmol) were added, and the resulting mixture was reacted at 25° C. for 1 hour. The solvent was concentrated under reduced pressure, and the resulting crude product was purified by column chromatography (ethyl acetate/petroleum ether=0-10%) to give compound 30-5. MS m/z: 347.0 [M+1]⁺; ¹H NMR (400 MHz, DMSO-d₆) δ 8.64 (s, 1H), 4.67 (s, 2H), 1.53 (s, 9H).

### Step 5: Synthesis of Compound 30-6

Compound 30-5 (3.07 g, 8.83 mmol) was added to dioxane (60 mL) and water (12 mL) under nitrogen. Compound 4-8 (2.16 g, 8.83 mmol), potassium carbonate (2.44 g, 17.66 mmol), and Pd(dppf)Cl₂ (646.26 mg, 883.22 μmol) were added. The resulting mixture was reacted at 70° C. for 2 hours, and cooled. The solvent was concentrated under reduced pressure to give a crude product. The crude product was added to water (10 mL) and ethyl acetate (20 mL). The mixture was stirred for 1 hour and vacuum filtered. The residue was washed with ethyl acetate (10 mL), and the residue was collected to give compound 30-6. MS m/z: 399.1 [M+1]⁺; ¹H NMR (400 MHz, DMSO-d₆) δ 8.95 (s, 1H), 8.43 (d, J=4.8 Hz, 1H), 7.65 (d, J=3.6 Hz, 1H), 7.41 (d, J=4.8 Hz, 1H), 6.69 (d, J=3.6 Hz, 1H), 4.92 (s, 2H), 3.89 (s, 3H), 1.51 (s, 9H).

### Step 6: Synthesis of Compound 30-7

Compound 30-6 (30 mg, 75.22 μmol) and compound 29-7 (19.70 mg, 90.26 mol) were added to dioxane (2 mL). Xantphos Pd G₄ (7.24 mg, 7.52 μmol) and cesium carbonate (73.52 mg, 225.65 μmol) were added. The resulting mixture was heated to 110° C. and reacted for 2 hours. Water (30 mL) was added to the reaction system, and the aqueous phase was extracted with ethyl acetate (30 mL×3). The layers were separated. The organic phase was dried over anhydrous sodium sulfate, filtered, and concentrated under reduced pressure to give compound 30-7. MS m/z: 581.2 [M+1]⁺.

### Step 7: Synthesis of a Trifluoroacetate of Compound 30

Compound 30-7 (50 mg, 86.11 μmol) was added to dioxane (2 mL) and trifluoroacetic acid (1 mL), and the resulting mixture was reacted at 25° C. for 2 hours. The reaction solution was concentrated under reduced pressure. The resulting crude product was purified by prep-HPLC (chromatography column Phenomenex Luna 80*30 mm*3 μm; mobile phase: [water (TFA)-acetonitrile]; acetonitrile %: 1%-25%, 8 min) to give a trifluoroacetate of compound 30. MS m/z: 481.2 [M+1]⁺; ¹H NMR (400 MHz, DMSO-d₆) δ 11.31 (br s, 1H), 10.23 (s, 1H), 10.03 (s, 1H), 9.30 (s, 1H), 8.38 (d, J=4.8 Hz, 1H), 7.82 (br d, J=8.4 Hz, 1H), 7.60 (d, J=3.4 Hz, 1H), 7.42 (d, J=5.0 Hz, 1H), 6.98 (d, J=8.2 Hz, 1H), 6.91 (d, J=3.6 Hz, 1H), 4.76 (s, 2H), 4.61-4.58 (m, 1H), 4.53-4.52 (m, 1H), 4.23-4.18 (m, 1H), 3.88 (s, 3H), 3.32 (s, 1H), 2.61-2.58 (m, 2H), 2.17-2.15 (m, 1H), 1.94-1.88 (m, 2H).

### Example 31

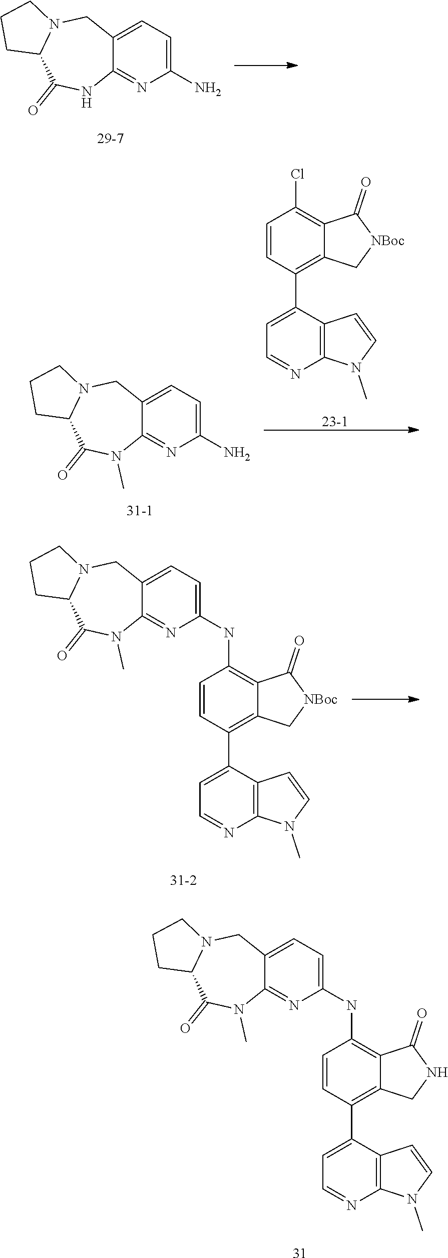

### Step 1: Synthesis of Compound 31-1

Compound 29-7 (50 mg, 229.09 μmol) and iodomethane (35.12 mg, 247.42 μmol, 15.40 μL) were added to DMF (1 mL). The resulting mixture was cooled to 0° C., and then sodium hydride (9.90 mg, 247.42 μmol, 60% purity) was added. The resulting mixture was stirred at 25° C. for 1 hour. The reaction solution was slowly added to a saturated aqueous solution of ammonium chloride (30 mL). The resulting mixture was extracted with ethyl acetate (30 mL×3). The organic phases were combined, dried over anhydrous sodium sulfate, and filtered. The filtrate was concentrated under reduced pressure to give compound 31-1. MS m/z: 232.9 [M+1]⁺.

### Step 2: Synthesis of Compound 31-2

Compound 23-1 (20 mg, 50.27 μmol) and compound 31-1 (14.01 mg, 60.32 mol) were added to dioxane (2 mL). Cesium carbonate (32.76 mg, 100.54 μmol), Pd(OAc)₂ (1.13 mg, 5.03 μmol), and Xantphos (5.82 mg, 10.05 μmol) were added. The resulting mixture was heated to 110° C. and reacted for 2 hours. The reaction solution was added to water (20 mL), and the resulting mixture was extracted with ethyl acetate (30 mL×3). The organic phases were combined, dried over anhydrous sodium sulfate, and filtered. The filtrate was concentrated under reduced pressure to give compound 31-2. MS m/z: 594.4 [M+1]⁺.

### Step 3: Synthesis of a Trifluoroacetate of Compound 31

Compound 31-2 (30 mg, 50.53 μmol) was added to DCM (2 mL) and trifluoroacetic acid (1 mL). The resulting mixture was reacted at 25° C. for 2 hours. The reaction solution was concentrated under reduced pressure and purified by prep-HPLC (chromatography column: Waters Xbridge BEH C18 100*30 mm*10 μm; mobile phase: [water (TFA)-acetonitrile]; acetonitrile %: 20%-50%, 8 min). The resulting product was concentrated to remove the organic solvent, and added to a saturated aqueous solution of sodium bicarbonate (10 mL). The resulting mixture was extracted with dichloromethane (10 mL×2). The organic phases were combined, dried over anhydrous sodium sulfate, and filtered. The filtrate was concentrated under reduced pressure, and methanol (0.2 mL) was added to dissolve the resulting product. Trifluoroacetic acid (5 microliters) was added, and deionized water (3 mL) was added to give a trifluoroacetate of compound 31. MS m/z: 494.4[M+1]⁺; ¹H NMR (400 MHz, DMSO-d₆) δ 10.50 (s, 1H), 8.94 (s, 1H), 8.62 (d, J=8.4 Hz, 1H), 8.33 (d, J=5.0 Hz, 1H), 7.83 (d, J=8.4 Hz, 2H), 7.60 (d, J=3.6 Hz, 1H), 7.29 (d, J=4.8 Hz, 1H), 7.00 (d, J=8.4 Hz, 1H), 6.47 (d, J=3.4 Hz, 1H), 4.50 (s, 3H), 3.70-3.65 (m, 3H), 3.54 (s, 3H), 3.17-3.16 (m, 2H), 2.26-2.16 (m, 2H), 1.94-1.84 (m, 2H), 1.26-1.24 (m, 2H).

### Example 32

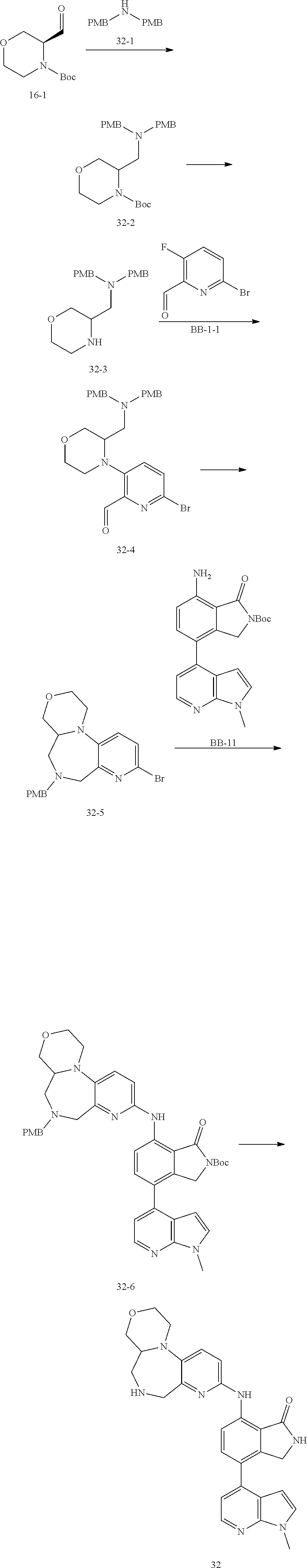

### Step 1: Synthesis of Compound 32-2

Compound 16-1 (5 g, 23.23 mmol) was added to DCM (120 mL) under nitrogen. Compound 32-1 (5.98 g, 23.23 mmol) and acetic acid (1.39 g, 23.23 mmol, 1.33 mL) were added. The resulting mixture was reacted at 20° C. for 1 hour. Then, NaBH(OAc)₃ (7.38 g, 34.84 mmol) was added, and the resulting mixture was reacted at 20° C. for 16 hours. A saturated aqueous solution of sodium bicarbonate (50 mL) was added to quench the reaction. The mixture was extracted with dichloromethane (50 mL×3). The organic phases were combined, washed with saturated brine (20 mL), dried over anhydrous sodium sulfate, and filtered. The filtrate was concentrated under reduced pressure. The resulting crude product was separated by column chromatography (ethyl acetate/petroleum ether=0%-20%) to give compound 32-2. MS m/z: 457.2 [M+1]⁺; ¹H NMR (400 MHz, CDCl₃) δ 7.25 (d, J=8.4 Hz, 4H), 6.84 (d, J=8.4 Hz, 4H), 4.30-3.96 (m, 1H), 3.90 (d, J=11.6 Hz, 1H), 3.80 (s, 6H), 3.73-3.42 (m, 7H), 3.41-3.28 (m, 1H), 2.68 (s, 3H), 1.48 (s, 9H).

### Step 2: Synthesis of Compound 32-3

Compound 32-2 (8.4 g, 18.40 mmol) was dissolved in DCM (40 mL) under nitrogen, and hydrochloric acid/methanol (4 M, 140 mL) was added. The resulting mixture was reacted at 20° C. for 16 hours. The solvent was concentrated under reduced pressure. Dichloromethane (40 mL) and aqueous ammonia (5 mL) were added. The mixture was dried over anhydrous sodium sulfate and filtered. The filtrate was concentrated under reduced pressure to give compound 32-3. MS m/z: 357.2 [M+1]⁺.

### Step 3: Synthesis of Compound 32-4

Compound 32-3 (4 g, 11.22 mmol) was dissolved in DMF (80 mL) under nitrogen. Compound BB-1-1 (2.75 g, 13.47 mmol) and DIEA (4.35 g, 33.66 mmol, 5.86 mL) were added. The resulting mixture was reacted at 100° C. for 16 hours. The mixture was cooled, and water (100 mL) was added to quench the reaction. The resulting mixture was extracted with ethyl acetate (50 mL×3). The organic phases were combined, washed with saturated brine (20 mL), dried over anhydrous sodium sulfate, and filtered. The filtrate was concentrated under reduced pressure. The resulting crude product was purified by column chromatography (ethyl acetate/petroleum ether=0%-50%) to give compound 32-4. MS m/z: 540.1 [M+1]⁺; ¹H NMR (400 MHz, CDCl₃) δ 9.83 (s, 1H), 7.45 (d, J=8.8 Hz, 1H), 6.98 (d, J=8.8 Hz, 1H), 6.92-6.86 (m, 4H), 6.85-6.78 (m, 4H), 4.07-4.03 (m, 1H), 3.91-3.74 (m, 8H), 3.67-3.56 (m, 1H), 3.54-3.41 (m, 3H), 3.10-2.97 (m, 3H), 2.86-2.81 (m, 1H), 2.67 (d, J=12.0 Hz, 1H), 2.53-2.48 (m, 1H).

### Step 4: Synthesis of Compound 32-5

Compound 32-4 (6 g, 11.10 mmol) was dissolved in trifluoroacetic acid (63.29 g, 555.10 mmol, 41.10 mL) under nitrogen, and the resulting mixture was reacted at 60° C. for 16 hours. The mixture was cooled and concentrated under reduced pressure. Dichloromethane (60 mL) and NaBH(OAc)₃ (7.06 g, 33.31 mmol) were added, and the resulting mixture was reacted at 25° C. for 2 hours. A saturated sodium bicarbonate solution (50 mL) was added to quench the reaction, and the resulting mixture was extracted with dichloromethane (50 mL×3). The organic phase was washed with saturated brine (20 mL), dried over anhydrous sodium sulfate, vacuum filtered, and concentrated under reduced pressure. The resulting crude product was purified by column chromatography (ethyl acetate/petroleum ether=0%-50%) to give compound 32-5. MS m/z: 404.0, 406.0 [M+1]⁺; ¹H NMR (400 MHz, CDCl₃) δ 7.24 (d, J=9.6 Hz, 3H), 7.09 (d, J=8.4 Hz, 1H), 6.85 (d, J=8.4 Hz, 2H), 4.04 (s, 2H), 3.89-3.81 (m, 5H), 3.71-3.63 (m, 3H), 3.59-3.51 (m, 1H), 3.35-3.27 (m, 2H), 3.26-3.17 (m, 1H), 3.09-3.01 (m, 1H), 2.63-2.54 (m, 1H).

### Step 5: Synthesis of Compound 32-6

Compound 32-5 (50 mg, 132.13 μmol) was dissolved in dioxane (2 mL) under nitrogen. Compound BB-11 (53.42 mg, 132.13 μmol), cesium carbonate (129.15 mg, 396.38 mol), Xantphos (15.29 mg, 26.43 μmol), and palladium acetate (2.97 mg, 13.21 μmol) were added. The resulting mixture was reacted at 110° C. for 2 hours. The mixture was cooled. The solvent was concentrated under reduced pressure. The resulting crude product was purified by column chromatography (ethyl acetate/petroleum ether=0%-100%) to give compound 32-6. MS m/z: 702.2 [M+1]⁺.

### Step 6: Synthesis of a Trifluoroacetate of Compound 32

Compound 32-6 (60 mg, 85.49 μmol) was dissolved in trifluoroacetic acid (6 mL) under nitrogen, and the resulting mixture was reacted at 80° C. for 72 hours. The mixture was concentrated under reduced pressure to give a crude product. The crude product was purified by prep-HPLC (chromatography column: Phenomenex luna C18 100*40 mm*3 μm; mobile phase: [water(TFA(-acetonitrile]; B %: 10%-55%, 8 min) to give a trifluoroacetate of compound 32. MS m/z: 482.2 [M+1]⁺; ¹H NMR (400 MHz, CD₃OD) δ 8.75 (d, J=8.8 Hz, 1H), 8.35 (d, J=5.2 Hz, 1H), 7.77 (d, J=8.8 Hz, 1H), 7.60 (d, J=8.8 Hz, 1H), 7.49 (d, J=3.6 Hz, 1H), 7.31 (d, J=5.2 Hz, 1H), 7.08 (d, J=8.8 Hz, 1H), 6.53 (d, J=3.6 Hz, 1H), 4.66-4.52 (m, 2H), 4.49 (s, 2H), 3.98-3.91 (m, 4H), 3.90-3.82 (m, 2H), 3.77-3.70 (m, 1H), 3.48 (s, 3H), 3.40-3.35 (m, 2H).

### Example 33

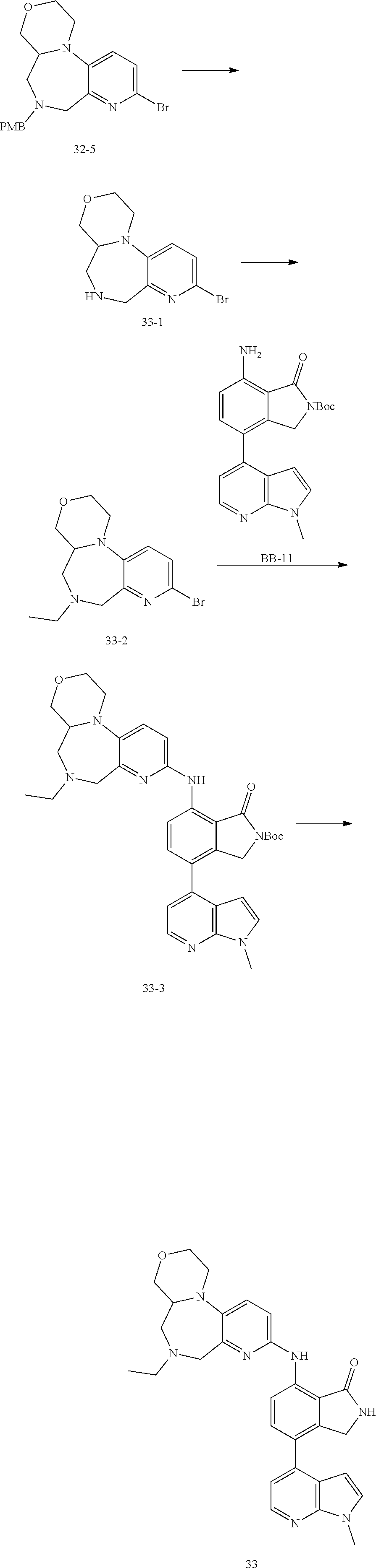

### Step 1: Synthesis of Compound 33-1

Compound 32-5 (1.0 g, 2.47 mmol) was added to trifluoroacetic acid (20.00 mL) under nitrogen, and the resulting mixture was reacted at 80° C. for 24 hours. The mixture was concentrated under reduced pressure, and methanol (20 mL) was added. Solid sodium bicarbonate was added to quench the reaction. The mixture was vacuum filtered. The residue was washed with methanol (20 mL). The filtrate was concentrated under reduced pressure. The resulting crude product was purified by column chromatography (methanol/dichloromethane=0%-20%) to give compound 33-1. MS m/z: 284.0 [M+1]⁺.

### Step 2: Synthesis of Compound 33-2

Compound 33-1 (100 mg, 351.92 μmol) was dissolved in DMF (1 mL) under nitrogen. Potassium carbonate (97.28 mg, 703.85 μmol) and iodoethane (54.89 mg, 351.92 μmol, 28.15 μL) were added. The resulting mixture was reacted at 25° C. for 16 hours. Water (10 mL) was added to quench the reaction, and the mixture was extracted with ethyl acetate (10 mL×3). The organic phase was washed with saturated brine (10 mL), dried over anhydrous sodium sulfate, vacuum filtered, and concentrated under reduced pressure. The resulting crude product was purified by column chromatography (ethyl acetate/petroleum ether=0%-70%) to give compound 33-2. MS m/z: 312.2 [M+1]⁺.

### Step 3: Synthesis of Compound 33-3

Compound BB-11 (40 mg, 105.70 μmol) was dissolved in dioxane (2 mL) under nitrogen. Compound 33-2 (33.00 mg, 105.70 μmol), cesium carbonate (103.32 mg, 317.11 mol), and methanesulfonato[9,9-dimethyl-4,5-bis(diphenylphosphino)xanthene](2′-methylamino-1,1′-biphenyl-2-yl)palladium(II) (10.17 mg, 10.57 μmol) were added. The resulting mixture was reacted at 110° C. for 2 hours. The mixture was cooled and concentrated under reduced pressure. The resulting crude product was purified by column chromatography (methanol/ethyl acetate=0%-10%) to give compound 33-3. MS m/z: 610.2 [M+1]⁺.

### Step 4: Synthesis of a Trifluoroacetate of Compound 33

Compound 33-3 (30 mg, 49.20 μmol) was dissolved in DCM (1.5 mL) under nitrogen, and trifluoroacetic acid (0.5 mL) was added. The resulting mixture was reacted at 25° C. for 2 hours. The mixture was concentrated under reduced pressure to give a crude product. The crude product was purified by prep-HPLC (chromatography column: Phenomenex Luna C18 150*30 mm*5 μm; mobile phase: [water (TFA)-acetonitrile]; B %: 1%-30%, 8 min) to give a trifluoroacetate of compound 33-3. MS m/z: 510.1 [M+1]⁺; ¹H NMR (400 MHz, CD₃OD) δ 8.75 (d, J=8.4 Hz, 1H), 8.36 (d, J=5.2 Hz, 1H), 7.78 (d, J=8.4 Hz, 1H), 7.59 (d, J=8.8 Hz, 1H), 7.51 (d, J=3.6 Hz, 1H), 7.33 (d, J=5.2 Hz, 1H), 7.09 (d, J=8.8 Hz, 1H), 6.55 (d, J=3.6 Hz, 1H), 4.80-4.62 (m, 3H), 4.49 (s, 2H), 4.01-3.82 (m, 7H), 3.72-3.59 (m, 2H), 3.53-3.40 (m, 4H), 1.47 (t, J=7.2 Hz, 3H).

### Example 34

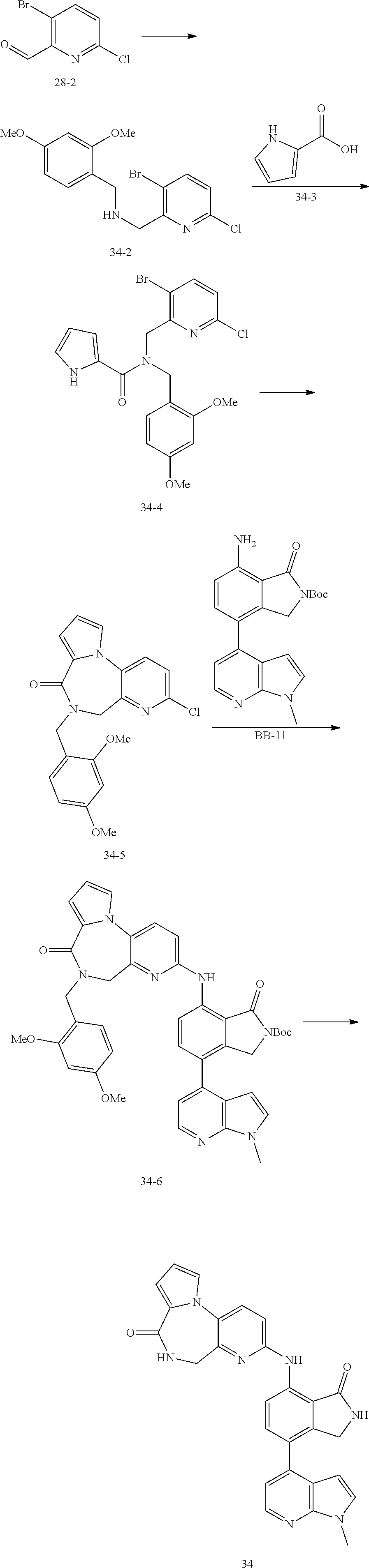

### Step 1: Synthesis of Compound 34-2

Compound 28-2 (1.5 g, 6.80 mmol) was added to DCM (15 mL) under nitrogen. 2,4-dimethoxybenzylamine (1.25 g, 7.48 mmol, 1.13 mL) and AcOH (408.59 mg, 6.80 mmol) were added. The resulting mixture was reacted at 25° C. for 1 hour. Then, NaBH(OAc)₃ (1.73 g, 8.17 mmol) was added, and the resulting mixture was reacted at 25° C. for 15 hours. A saturated aqueous solution of sodium bicarbonate (50 mL) was added to quench the reaction. The mixture was extracted with dichloromethane (50 mL×3). The organic phase was washed with saturated brine (20 mL), dried over anhydrous sodium sulfate, and filtered. The filtrate was concentrated under reduced pressure. The resulting crude product was purified by column chromatography (ethyl acetate/petroleum ether=0%-30%) to give compound 34-2. MS m/z: 371.0 [M+1]⁺; ¹H NMR (400 MHz, CDCl₃) δ 7.72 (d, J=8.4 Hz, 1H), 7.18 (d, J=8.8 Hz, 1H), 7.06 (d, J=8.4 Hz, 1H), 6.46-6.39 (m, 2H), 3.98 (s, 2H), 3.82 (s, 3H), 3.81 (s, 2H), 3.80 (s, 3H).

### Step 2: Synthesis of Compound 34-4

Compound 34-2 (240 mg, 2.16 mmol) was dissolved in DMF (6 mL) under nitrogen. DIEA (6.48 mmol, 1.13 mL), HATU (1.23 g, 3.24 mmol), and compound 34-3 (802.87 mg, 2.16 mmol) were added. The resulting mixture was reacted at 25° C. for 2 hours. Water (20 mL) was added to quench the reaction, and the mixture was extracted with ethyl acetate (10 mL×3). The organic phase was washed with saturated brine (10 mL), dried over anhydrous sodium sulfate, and filtered. The filtrate was concentrated under reduced pressure. The resulting crude product was purified by column chromatography (ethyl acetate/petroleum ether=0%-30%) to give compound 34-4. MS m/z: 485.9 [M+Na]⁺.

### Step 3: Synthesis of Compound 34-5

Compound 34-4 (604 mg, 1.30 mmol) was dissolved in DMF (6 mL) under nitrogen. 1,10-phenanthroline (46.84 mg, 259.93 μmol), cesium carbonate (1.27 g, 3.90 mmol), and copper(I) iodide (24.75 mg, 129.97 μmol) were added. The resulting mixture was reacted at 140° C. for 16 hours. The mixture was cooled, and water (30 mL) was added to quench the reaction. The mixture was extracted with ethyl acetate (20 mL×3). The organic phases were combined, washed with saturated brine (20 mL×2), dried over anhydrous sodium sulfate, vacuum filtered, and concentrated under reduced pressure. The resulting crude product was purified by column chromatography (ethyl acetate/petroleum ether=0%-50%) to give compound 34-5. MS m/z: 384.0 [M+1]⁺; ¹H NMR (400 MHz, CDCl₃) δ 7.59 (d, J=8.4 Hz, 1H), 7.35 (d, J=8.4 Hz, 1H), 7.23 (d, J=8.4 Hz, 1H), 7.18-7.16 (m, 1H), 7.06-7.05 (m, 1H), 6.50-6.38 (m, 3H), 4.76 (s, 2H), 4.48 (s, 2H), 3.82 (s, 3H), 3.76 (s, 3H).

### Step 4: Synthesis of Compound 34-6

Compound 34-5 (70 mg, 184.98 μmol) was dissolved in dioxane (4 mL) under nitrogen. Compound BB-11 (71.00 mg, 184.98 μmol), cesium carbonate (180.81 mg, 554.93 mol), 2-dicyclohexylphosphino-2′-(N,N-dimethylamino)biphenyl (14.56 mg, 37.00 μmol), and palladium acetate (4.15 mg, 18.50 μmol) were added. The resulting mixture was reacted at 110° C. for 4 hours. The mixture was cooled and concentrated under reduced pressure. The resulting crude product was purified by column chromatography (ethyl acetate/petroleum ether=0%-100%) to give compound 34-6. MS m/z: 726.1 [M+1]⁺.

### Step 5: Synthesis of Compound 34

Compound 34-6 (60 mg, 82.67 μmol) was dissolved in trifluoroacetic acid (4 mL) under nitrogen, and the resulting mixture was reacted at 25° C. for 16 hours. Then, the mixture was heated to 60° C. and reacted for 6 hours. The mixture was concentrated under reduced pressure to give a crude product. The crude product was purified by prep-HPLC (chromatography column: Phenomenex C18 80*30 mm*3 μm; mobile phase: [water (TFA)-acetonitrile]; B %: 10%-40%, 8 min), and concentrated under reduced pressure. Aqueous ammonia was added to adjust the pH to 8, and the resulting mixture was extracted with dichloromethane (20 mL×3). The organic phases were combined and concentrated under reduced pressure to give compound 34. MS m/z: 476.1 [M+1]⁺; ¹H NMR (400 MHz, DMSO-d₆) δ 10.40-10.33 (m, 1H), 8.87 (s, 1H), 8.76 (d, J=8.4 Hz, 1H), 8.42-8.37 (m, 1H), 8.32 (d, J=4.8 Hz, 1H), 7.91 (d, J=8.8 Hz, 1H), 7.78 (d, J=8.4 Hz, 1H), 7.59 (d, J=3.6 Hz, 1H), 7.50 (d, J=2.0 Hz, 1H), 7.28 (d, J=4.8 Hz, 1H), 7.16 (d, J=8.8 Hz, 1H), 6.90-6.89 (m, 1H), 6.47 (d, J=3.6 Hz, 1H), 6.44-6.41 (m, 1H), 4.48 (s, 2H), 4.24 (d, J=4.8 Hz, 2H), 3.87 (s, 3H).

### Example 35

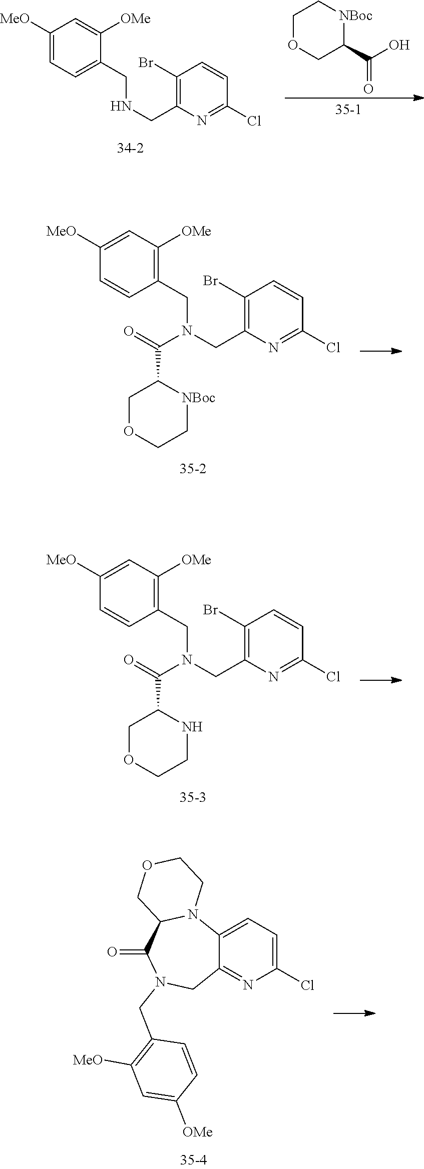 [SMILES: COc1ccc(CNCc2nc(Cl)ccc2Br)c(OC)c1 ; CC(C)(C)OC(=O)N1CCOC[C@@H]1C(=O)O ; COc1ccc(CN(Cc2nc(Cl)ccc2Br)C(=O)[C@H]2COCCN2C(=O)OC(C)(C)C)c(OC)c1 ; COc1ccc(CN(Cc2nc(Cl)ccc2Br)C(=O)[C@H]2COCCN2)c(OC)c1 ; COc1ccc(CN2Cc3nc(Cl)ccc3N3CCOC[C@@H]3C2=O)c(OC)c1]

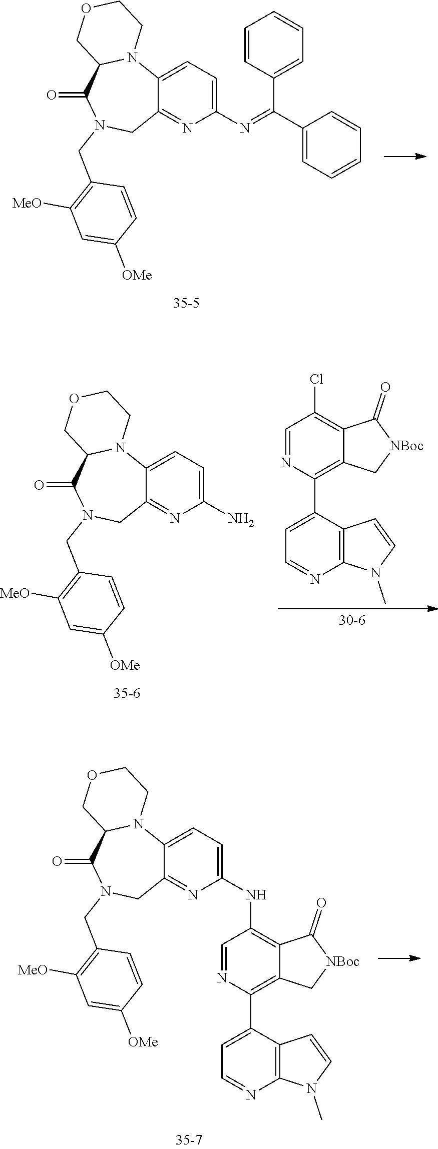 [SMILES: COc1ccc(CN2Cc3nc(N=C(c4ccccc4)c4ccccc4)ccc3N3CCOC[C@@H]3C2=O)c(OC)c1 ; COc1ccc(CN2Cc3nc(N)ccc3N3CCOC[C@@H]3C2=O)c(OC)c1 ; Cn1ccc2c(-c3ncc(Cl)c4c3CN(C(=O)OC(C)(C)C)C4=O)ccnc21 ; COc1ccc(CN2Cc3nc(Nc4cnc(-c5ccnc6c5ccn6C)c5c4C(=O)N(C(=O)OC(C)(C)C)C5)ccc3N3CCOC[C@@H]3C2=O)c(OC)c1]

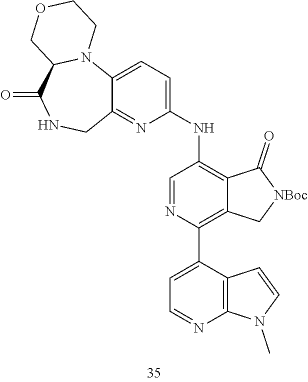 [SMILES: Cn1ccc2c(-c3ncc(Nc4ccc5c(n4)CNC(=O)[C@H]4COCCN54)c4c3CN(C(=O)OC(C)(C)C)C4=O)ccnc21]

### Step 1: Synthesis of Compound 35-2

Compound 35-1 (125 mg, 540.55 μmol) was dissolved in DCM (2 mL) under nitrogen. DIEA (209.59 mg, 1.62 mmol, 282.46 μL), HATU (308.30 mg, 810.83 μmol), and compound 34-2 (200.90 mg, 540.55 μmol) were added, and the resulting mixture was reacted at 25° C. for 2 hours. Water (20 mL) was added to quench the reaction, and the mixture was extracted with ethyl acetate (10 mL×3). The organic phase was washed with saturated brine (10 mL), dried over anhydrous sodium sulfate, and filtered. The filtrate was concentrated under reduced pressure. The resulting crude product was purified by column chromatography (ethyl acetate/petroleum ether=0%-50%) to give compound 35-2. MS m/z: 605.9, 607.9 [M+Na]⁺.

### Step 2: Synthesis of Compound 35-3

Compound 35-2 (600 mg, 1.03 mmol) was dissolved in DCM (6 mL) under nitrogen, and zinc bromide (1.16 g, 5.13 mmol, 256.69 μL) was added. The resulting mixture was reacted at 25° C. for 2 hours, and vacuum filtered. The residue was washed with dichloromethane (10 mL). The filtrate was collected, and water (10 mL) was added. The resulting mixture was extracted with dichloromethane (10 mL×3). The organic phases were combined, washed with saturated brine (10 mL), dried over anhydrous sodium sulfate, and filtered. The filtrate was concentrated under reduced pressure. The resulting crude product was purified by column chromatography (ethyl acetate/petroleum ether=0%-100%) to give compound 35-3. MS m/z: 484.0, 486.0 [M+1]⁺.

### Step 3: Synthesis of Compound 35-4

Compound 35-3 (320 mg, 660.11 μmol) was dissolved in dioxane (6 mL) under nitrogen. Cesium carbonate (645.23 mg, 1.98 mmol), 2-di-tert-butylphosphino-2′-(N,N-dimethylamino)biphenyl (51.96 mg, 132.02 μmol), and palladium acetate (14.82 mg, 66.01 mol) were added. The resulting mixture was reacted at 110° C. for 2 hours. The mixture was cooled and vacuum filtered. The residue was washed with dichloromethane (10 mL). The filtrate was concentrated under reduced pressure. The resulting crude product was purified by column chromatography (ethyl acetate/petroleum ether=0%-70%) to give compound 35-4. MS m/z: 404.1 [M+1]⁺.

### Step 4: Synthesis of Compound 35-5

Compound 35-4 (79 mg, 195.61 μmol) was dissolved in dioxane (4 mL) under nitrogen. Benzophenone imine (42.54 mg, 234.74 μmol, 39.39 μL), sodium tert-butoxide (37.60 mg, 391.23 μmol), BINAP (24.36 mg, 39.12 μmol), and Pd₂(dba)₃ (17.91 mg, 19.56 μmol) were added. The resulting mixture was reacted at 110° C. for 2 hours. The mixture was cooled, and a saturated sodium bicarbonate solution (10 mL) was added to quench the reaction. The mixture was extracted with ethyl acetate (10 mL×3). The organic phases were combined, dried over anhydrous sodium sulfate, and filtered. The filtrate was concentrated under reduced pressure to give compound 35-5. MS m/z: 549.2 [M+1]⁺.

### Step 5: Synthesis of Compound 35-6

Compound 35-5 (107 mg, 195.03 μmol) was dissolved in DCM (2 mL) under nitrogen, and trifluoroacetic acid (667.14 mg, 5.85 mmol, 433.21 μL) was added. The resulting mixture was reacted at 25° C. for 1 hour. The mixture was concentrated under reduced pressure. Dichloromethane (10 mL) and a saturated sodium carbonate solution (10 mL) were added. The mixture was extracted with dichloromethane (10 mL×3). The organic phases were combined, dried over anhydrous sodium sulfate, and filtered. The filtrate was concentrated under reduced pressure. The resulting crude product was purified by column chromatography (ethyl acetate/petroleum ether=0%-100%) to give compound 35-6. MS m/z: 385.2 [M+1]⁺.

### Step 6: Synthesis of Compound 35-7

Compound 30-6 (65 mg, 162.97 μmol) was dissolved in dioxane (3 mL) under nitrogen. Compound 35-6 (62.65 mg, 162.97 μmol), cesium carbonate (159.30 mg, 488.92 mol), and methanesulfonato[9,9-dimethyl-4,5-bis(diphenylphosphino)xanthene](2′-methylamino-1,1′-biphenyl-2-yl)palladium(II) (15.68 mg, 16.30 μmol) were added. The resulting mixture was reacted at 110° C. for 2 hours. The mixture was cooled, and the solvent was concentrated under reduced pressure. The resulting crude product was purified by column chromatography (ethyl acetate/petroleum ether=0%-100%) to give compound 35-7. MS m/z: 747.2 [M+1]⁺.

### Step 7: Synthesis of a Trifluoroacetate of Compound 35

Compound 35-7 (69 mg, 92.39 μmol) was dissolved in trifluoroacetic acid (2.5 mL) under nitrogen, and the resulting mixture was reacted at 60° C. for 16 hours. The solvent was concentrated under reduced pressure to give a crude product. The crude product was purified by prep-HPLC (chromatography column: Phenomenex Luna C18 75*30 mm*3 μm; mobile phase: [water (trifluoroacetic acid)-acetonitrile]; acetonitrile %: 10%-40%, 8 min) to give a trifluoroacetate of compound 35. MS m/z: 497.2[M+1]⁺; ¹H NMR (400 MHz, DMSO-d₆) δ 10.01 (s, 1H), 9.68 (s, 1H), 9.20 (s, 1H), 8.40-8.35 (m, 2H), 7.67-7.54 (m, 2H), 7.39 (d, J=4.8 Hz, 1H), 7.13 (d, J=8.8 Hz, 1H), 6.90 (d, J=3.6 Hz, 1H), 4.72 (s, 3H), 4.06-4.00 (m, 1H), 3.97-3.90 (m, 2H), 3.87 (s, 3H), 3.78-3.71 (m, 1H), 3.70-3.63 (m, 1H), 3.53-3.49 (m, 1H), 3.24-3.21 (m, 1H), 2.99 (d, J=11.6 Hz, 1H).

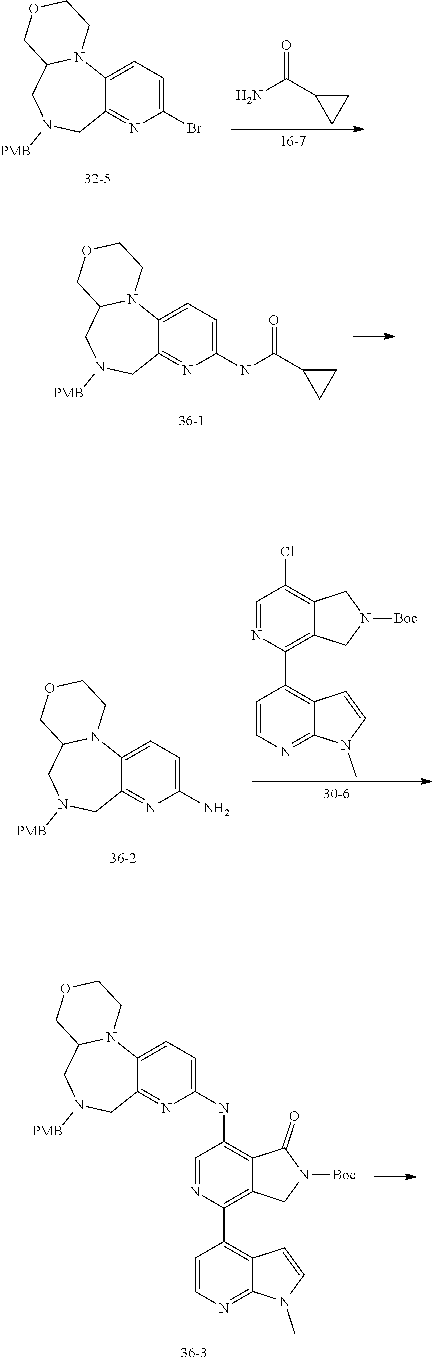

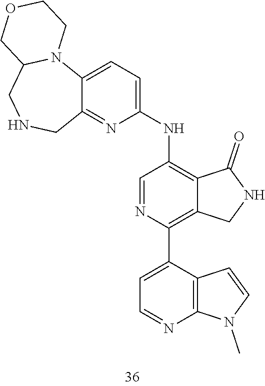 [SMILES: Cn1ccc2c(-c3ncc(Nc4ccc5c(n4)CNCC4COCCN54)c4c3CNC4=O)ccnc21]

### Step 1: Synthesis of Compound 36-1

Compound 32-5 (1 g, 2.47 mmol) was dissolved in dioxane (2 mL) in a dry vial. Compound 16-7 (421.00 mg, 4.95 mmol) was added to the solution, and cesium carbonate (2.42 g, 7.42 mmol) was added. The resulting mixture was stirred for 15 minutes, and then pH was measured to be greater than 9. Then, Pd₂(dba)₃ (421.00 mg, 4.95 mmol) and 4,5-bis(diphenylphosphino)-9,9-dimethylxanthene (286.23 mg, 494.68 μmol) were added. The reaction solution was stirred at 110° C. under nitrogen for 2 hours. The reaction solution was filtered. The filtrate was concentrated, and separated by column chromatography (ethyl acetate/petroleum ether=0%-5%) to give compound 36-1. MS m/z: 409.1[M+1]⁺; ¹H NMR (400 MHz, CDCl₃) δ 8.51 (s, 1H), 8.05-8.01 (m, 1H), 7.35-7.27 (m, 2H), 6.92-6.88 (m, 2H), 3.95-3.78 (m, 7H), 3.74-3.55 (m, 4H), 3.37-3.18 (m, 3H), 3.07-3.01 (m, 1H), 2.68-2.64 (m, 1H), 1.62-1.57 (m, 1H), 1.11-1.02 (m, 4H).

### Step 2: Synthesis of Compound 36-2

Compound 36-1 (750 mg, 1.84 mmol) was dissolved in water (5 mL) in a dry vial, and methanol (20 mL) was added. Sodium hydroxide (1.10 g, 27.54 mmol) was added to the solution. The reaction solution was heated to 80° C., stirred and reacted for 16 hours under nitrogen. The resulting mixture was concentrated under reduced pressure, and the mixture was extracted with dichloromethane (20 mL×5). The organic phases were combined, washed with saturated brine (20 mL), dried over anhydrous sodium sulfate, and filtered. The filtrate was concentrated under reduced pressure to give compound 36-2. MS m/z: 341.0[M+1]⁺; ¹H NMR (400 MHz, CDCl₃) δ ppm 7.27-7.24 (m, 2H), 7.14 (d, J=8.4 Hz, 1H), 6.87-6.84 (m, 2H), 6.40 (d, J=8.4 Hz, 1H), 4.19 (s, 2H), 4.02-3.82 (m, 2H), 3.80 (s, 3H), 3.67-3.664 (m, 1H), 3.63-3.61 (m, 1H), 3.41-3.39 (m, 1H), 3.11-3.09 (m, 1H), 3.06-3.04 (m, 2H), 2.85 (m, 2H), 2.61-2.59 (m, 1H), 2.38-2.36 (m, 1H), 2.05-2.03 (m, 1H).

### Step 3: Synthesis of Compound 36-3

Compound 36-2 (180 mg, 528.76 μmol) and compound 30-6 (210.89 mg, 528.76 mol) were dissolved in dioxane (3 mL) in a dry vial, and cesium carbonate (516.84 mg, 1.59 mmol) was added. The resulting mixture was stirred for 15 minutes, and then the pH was measured to be greater than 9. Then, methanesulfonato[4,5-bis(diphenylphosphino)-9,9-dimethylxanthene](2′-methylamino-1,1′-biphenyl-2-yl)palladium(II) (101.77 mg, 105.75 μmol) was added under nitrogen, and the reaction solution was stirred and reacted at 110° C. under nitrogen for 2 hours. The reaction solution was concentrated, and ethyl acetate (20 mL) and water (20 mL) were added. The layers were separated. The aqueous phase was extracted with ethyl acetate (10 mL×2). The organic phases were combined, dried over anhydrous sodium sulfate, and filtered. The filtrate was concentrated. The crude product was separated by column chromatography (ethyl acetate/petroleum ether=0%-15%) to give compound 36-3. MS m/z: 703.3[M+1]⁺; ¹H NMR (400 MHz, CDCl₃) δ ppm 10.30 (s, 1H), 9.42 (s, 1H), 8.45 (d, J=3.2 Hz, 1H), 7.30-7.27 (m, 4H), 7.21-7.19 (m, 1H), 6.89-6.82 (m, 4H), 4.95 (s, 2H), 4.17-4.11 (m, 1H), 3.97 (s, 3H), 3.95-3.80 (m, 9H), 3.77-3.74 (m, 1H), 3.38-3.14 (m, 3H), 3.10-3.00 (m, 1H), 2.68-2.61 (m, 1H), 1.62 (s, 9H).

### Step 4: Synthesis of a Trifluoroacetate of Compound 36

Compound 36-3 (42 mg, 59.76 μmol) was dissolved in trifluoroacetic acid (2 mL) in a dry vial, and the reaction solution was stirred at 85° C. for 48 hours. The reaction solution was concentrated to give a crude product. The crude product was purified by prep-HPLC (chromatography column: Phenomenex Luna C18 75*30 mm*3 μm; mobile phase: [water (trifluoroacetic acid)-acetonitrile]; acetonitrile %: 5%-35%, 8 min) to give a trifluoroacetate of compound 36. MS m/z: 483.22[M+1]⁺; ¹H NMR (400 MHz, DMSO-d₆) δ ppm 10.01 (s, 1H), 9.71 (s, 1H), 9.24-9.16 (m, 2H), 8.36 (d, J=4.8 Hz, 1H), 7.60-7.57 (m, 2H), 7.39 (d, J=4.8 Hz, 1H), 7.19 (d, J=8.8 Hz, 1H), 6.89 (d, J=3.2 Hz, 1H), 4.72 (s, 2H), 4.56-4.50 (m, 2H), 3.87 (s, 3H), 3.68-3.46 (m, 5H), 3.27-3.14 (m, 4H).

### Example 37

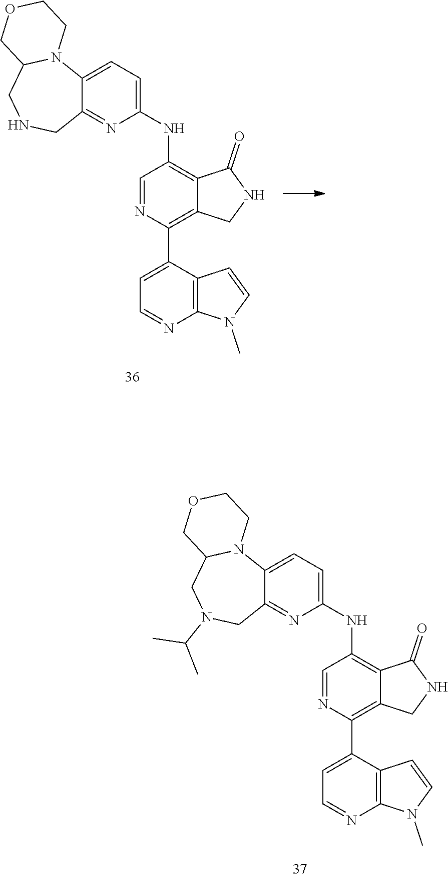 [SMILES: Cn1ccc2c(-c3ncc(Nc4ccc5c(n4)CNCC4COCCN54)c4c3CNC4=O)ccnc21 ; CC(C)N1Cc2nc(Nc3cnc(-c4ccnc5c4ccn5C)c4c3C(=O)NC4)ccc2N2CCOCC2C1]

### Step 1: Synthesis of a Trifluoroacetate of Compound 37

Compound 36 (6.00 mg, 12.43 μmol) and acetone (2.17 mg, 37.30 μmol, 2.74 μL) were added to methanol (1 mL) in a dry vial. Potassium acetate (2.44 mg, 24.87 μmol) and acetic acid (746.68 g, 12.43 μmol) were added to the solution, and the resulting mixture was stirred and reacted for 10 minutes. Then, NaBH(OAc)₃ (7.91 mg, 37.30 μmol) was added, and the reaction solution was stirred and reacted at 25° C. for 1 hour. The reaction solution was concentrated to give a crude product. The crude product was purified by prep-HPLC (chromatography column: Phenomenex Luna C18 75*30 mm*3 am; mobile phase: [water (trifluoroacetic acid)-acetonitrile]; acetonitrile %: 10%-40%, 8 min) to give a trifluoroacetate of compound 37. MS m/z: 525.3 [M+1]⁺.

### Example 38

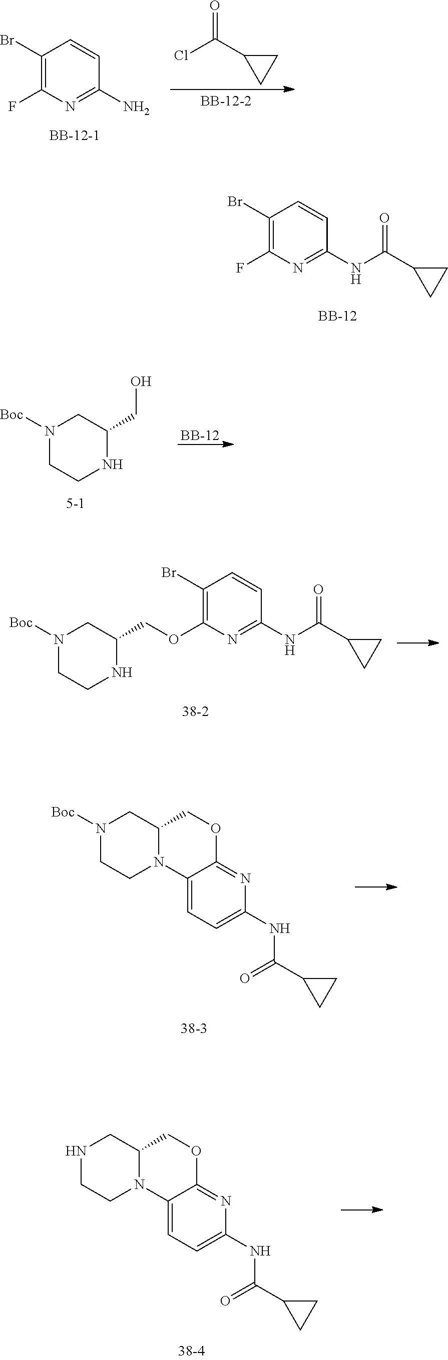

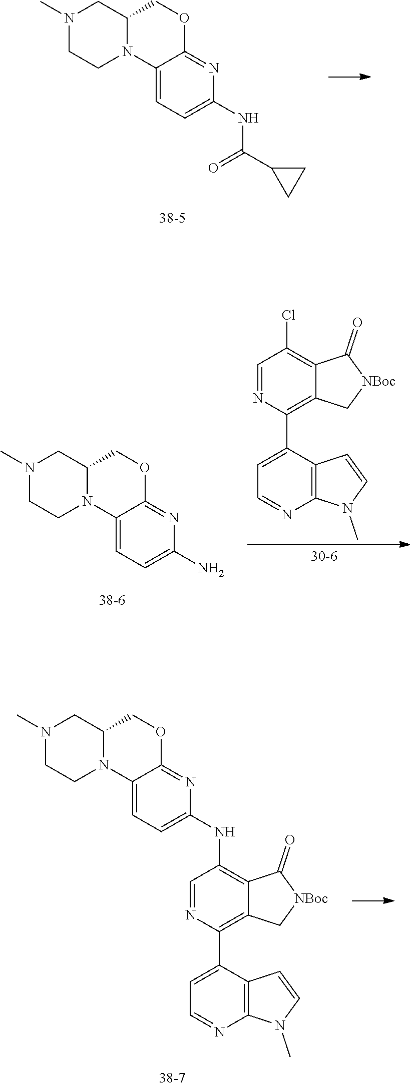 [SMILES: CN1CCN2c3ccc(NC(=O)C4CC4)nc3OC[C@H]2C1 ; CN1CCN2c3ccc(N)nc3OC[C@H]2C1 ; Cn1ccc2c(-c3ncc(Cl)c4c3CN(C(=O)OC(C)(C)C)C4=O)ccnc21 ; CN1CCN2c3ccc(Nc4cnc(-c5ccnc6c5ccn6C)c5c4C(=O)N(C(=O)OC(C)(C)C)C5)nc3OC[C@H]2C1]

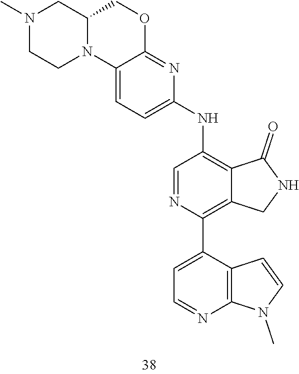 [SMILES: CN1CCN2c3ccc(Nc4cnc(-c5ccnc6c5ccn6C)c5c4C(=O)NC5)nc3OC[C@H]2C1]

### Step 1: Synthesis of Compound BB-12

Compound BB-12-1 (30 g, 157.07 mmol) and triethylamine (47.68 g, 471.20 mmol, 65.59 mL) were added to DCM (300 mL), and the mixture was cooled to 0° C. Compound BB-12-2 (49.26 g, 471.20 mmol, 42.83 mL) was added, and the resulting mixture was reacted at 25° C. for 16 hours. The reaction solution was poured into a saturated aqueous solution of ammonium chloride (300 mL), and the layers were separated. The aqueous phase was extracted with dichloromethane (50 mL×2). The organic phases were combined, washed with water (50 mL×2), dried over anhydrous sodium sulfate, filtered, and concentrated to give a crude product. The crude product was added to acetonitrile (200 mL). The mixture was stirred for 3 hours and then filtered. The filter cake was dried to give compound BB-12. MS m/z: 258.9, 280.9 [M+1]⁺; ¹H NMR (400 MHz, CDCl₃) δ 8.06 (s, 1H), 7.94-7.92 (m, 1H), 7.85-7.80 (m, 1H), 1.49-1.47 (m, 1H), 1.07-1.04 (m, 2H), 0.89-0.85 (m, 2H).

### Step 2: Synthesis of Compound 38-2

Compound BB-12 (0.5 g, 1.93 mmol) and potassium tert-butoxide (866.25 mg, 7.72 mmol) were added to dimethyl sulfoxide (5 mL). Compound 5-1 (417.40 mg, 1.93 mmol) was added, and the resulting mixture was reacted at 25° C. for 16 hours. The reaction solution was poured into a saturated aqueous solution of sodium carbonate (20 mL) to quench the reaction. The layers were separated. The aqueous phase was extracted with ethyl acetate (50 mL). The organic phase was dried over anhydrous sodium sulfate, filtered and concentrated to give a crude product. The crude product was purified by column chromatography (methanol/dichloromethane=0%-10%) to give compound 38-2. MS m/z:455.0, 456.9 [M+1]⁺; ¹H NMR (400 MHz, CDCl₃) δ 8.17 (s, 1H), 7.76-7.74 (m, 1H), 7.68-7.66 (m, 1H), 4.35-3.93 (m, 4H), 3.12-2.78 (m, 5H), 2.62 (s, 1H), 1.87 (s, 1H), 1.47 (s, 9H), 1.12-1.09 (m, 2H). 0.89-0.85 (m, 2H).

### Step 3: Synthesis of Compound 38-3

4,5-bis(diphenylphosphino)-9,9-dimethylxanthene (177.90 mg, 307.46 μmol) and cesium carbonate (1.00 g, 3.07 mmol) were added to a solution of compound 38-2 (0.7 g, 1.54 mmol) in dioxane (10 mL). The reaction system was purged with nitrogen three times. Palladium acetate (34.51 mg, 153.73 μmol) was added, and the mixture was reacted at 110° C. under nitrogen for 2 hours. The reaction solution was concentrated to give a crude product. The crude product was purified by column chromatography (methanol/dichloromethane=0%-10%) to give compound 38-3. MS m/z: 375.0 [M+1]⁺; ¹H NMR (400 MHz, CDCl₃) δ 7.90 (s, 1H), 7.60-7.40 (m, 1H), 7.09-7.07 (m, 1H), 4.39-4.36 (m, 1H), 4.14-4.09 (m, 3H), 3.59-3.56 (m, 1H), 3.05-3.00 (m, 2H), 2.70-2.67 (m, 2H), 1.67 (s, 1H), 1.49 (s, 9H), 11.07-1.05 (m, 2H). 0.87-0.83 (m, 2H).

### Step 4: Synthesis of a Hydrochloride of Compound 38-4

Compound 38-3 (0.38 g, 1.01 mmol) was added to hydrochloric acid/ethyl acetate (4 M, 7.6 mL), and the resulting mixture was reacted at 25° C. for 2 hours. The reaction system was concentrated to give a hydrochloride of compound 38-4. MS m/z: 275.4 [M+1]⁺.

### Step 5: Synthesis of Compound 38-5

The hydrochloride of compound 38-4 (0.33 g) was dissolved in DCM (3 mL). 37% aqueous solution of formaldehyde (861.70 mg, 10.62 mmol, 790.56 μL) and acetic acid (637.66 mg, 10.62 mmol, 607.30 μL) were added. The resulting mixture was reacted at 25° C. for 0.5 hours. NaBH(OAc)₃ (450.10 mg, 2.12 mmol) was added, and the mixture was reacted at 25° C. for 0.5 hours. Water (5 mL), dichloromethane (5 mL), and aqueous ammonia (5 mL) were added, and the layers were separated. The organic phase was dried over anhydrous sodium sulfate, filtered, and concentrated. The resulting crude product was purified by column chromatography (methanol/dichloromethane=10%) to give compound 38-5. MS m/z: 289.4 [M+1]⁺.

### Step 6: Synthesis of Compound 38-6

Compound 38-5 (50 mg, 173.40 μmol) was added to methanol (1 mL) and water (0.25 mL). Sodium hydroxide (69.36 mg, 1.73 mmol) was added, and the resulting mixture was reacted at 80° C. for 12 hours. The reaction system was concentrated, and water (2 mL) was added. The aqueous phase was extracted with a mixture of dichloromethane and methanol (10:1, 10 mL). The organic phase was dried over anhydrous sodium sulfate, filtered, and concentrated under reduced pressure to give compound 38-6. MS m/z: 221.4 [M+1]⁺.

### Step 7: Synthesis of Compound 38-7

Compound 30-6 (50 mg, 125.36 μmol) was added to dioxane (1 mL) under nitrogen. Compound 38-6 (30.38 mg, 137.90 μmol), methanesulfonato[9,9-dimethyl-4,5-bis(diphenylphosphino)xanthene](2′-methylamino-1,1′-biphenyl-2-yl)palladium(II) (12.06 mg, 12.54 μmol), and cesium carbonate (81.69 mg, 250.73 μmol) were added, and the mixture was reacted at 110° C. for 2 hours. Water (5 mL) was added, and the mixture was extracted with ethyl acetate (5 mL). The organic phase was dried over anhydrous sodium sulfate, filtered and concentrated. The resulting crude product was purified by column chromatography (methanol/dichloromethane=5%-10%) to give compound 38-7. MS m/z: 583.2 [M+1]⁺.

### Step 8: Synthesis of a Trifluoroacetate of Compound 38

Compound 38-7 (50 mg, 85.81 μmol) was dissolved in DCM (1 mL) under nitrogen, and trifluoroacetic acid (1.00 mL) was added. The resulting mixture was reacted at 25° C. for 1 hour. The reaction system was concentrated under reduced pressure to give a crude product. The crude product was purified by prep-HPLC (chromatography column: Phenomenex luna C18 100*40 mm*3 um; mobile phase: [(TFA)-acetonitrile]; acetonitrile %: 1%-40%, 8 min) to give a trifluoroacetate of compound 38. MS m/z: 483.5 [M+1]⁺; ¹H NMR (400 MHz, DMSO-d₆) δ 9.87 (s, 1H), 9.52 (s, 1H), 9.18 (s, 1H), 8.36 (d, J=5.0 Hz, 1H), 7.60-7.54 (m, 1H), 7.49-7.43 (m, 1H), 7.38 (d, J=5.0 Hz, 1H), 6.94-6.90 (m, 1H), 6.77 (d, J=8.4 Hz, 1H), 4.72 (s, 2H), 4.53-4.46 (m, 1H), 4.21-4.16 (m, 1H), 4.09-4.06 (m, 1H), 3.88 (s, 3H), 3.58-3.52 (m, 2H), 3.24-3.06 (m, 2H), 2.97-2.93 (m, 2H), 2.89 (s, 3H).

### Example 39

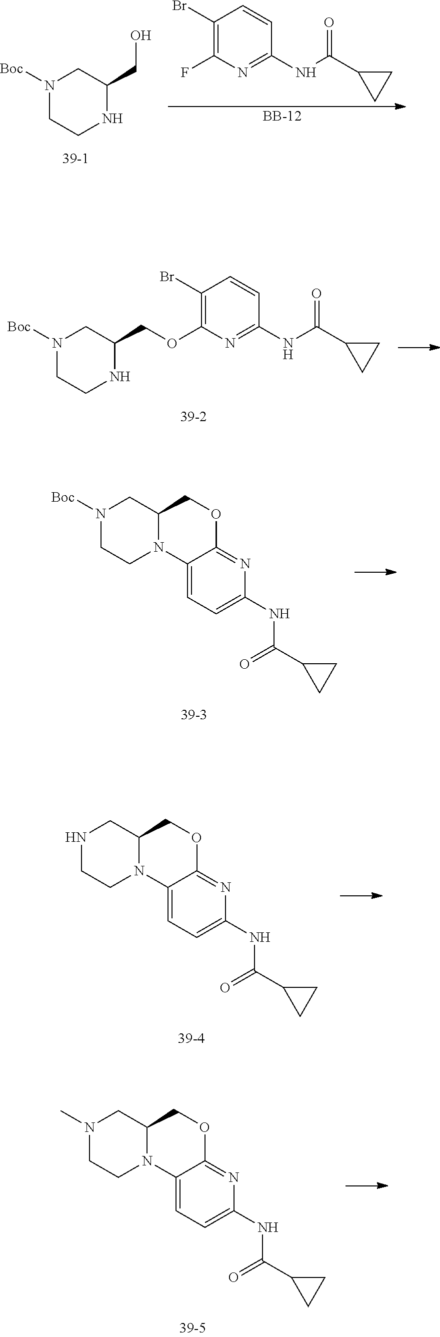

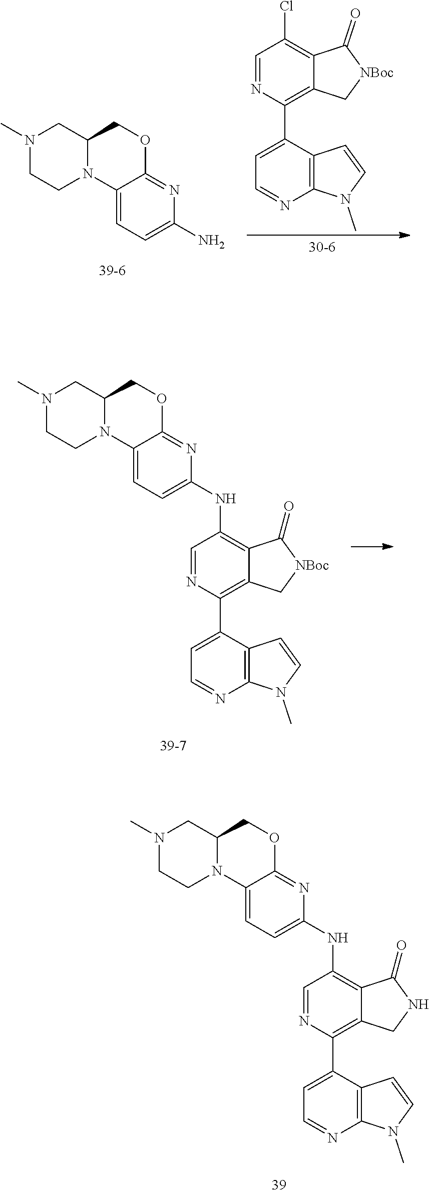 [SMILES: CN1CCN2c3ccc(N)nc3OC[C@@H]2C1 ; Cn1ccc2c(-c3ncc(Cl)c4c3CN(C(=O)OC(C)(C)C)C4=O)ccnc21 ; CN1CCN2c3ccc(Nc4cnc(-c5ccnc6c5ccn6C)c5c4C(=O)N(C(=O)OC(C)(C)C)C5)nc3OC[C@@H]2C1 ; CN1CCN2c3ccc(Nc4cnc(-c5ccnc6c5ccn6C)c5c4C(=O)NC5)nc3OC[C@@H]2C1]

### Step 1: Synthesis of Compound 39-2

Compound BB-12 (1 g, 3.86 mmol) and potassium tert-butoxide (1.73 g, 15.44 mmol) were added to dimethyl sulfoxide (10 mL). Compound 39-1 (834.80 mg, 3.86 mmol) was added, and the mixture was reacted at 25° C. for 16 hours. The reaction solution was poured into a saturated aqueous solution of sodium carbonate (100 mL) to quench the reaction. The layers were separated. The aqueous phase was extracted with ethyl acetate (50 mL*3). The organic phase was washed with saturated brine (50 mL*3), dried over anhydrous sodium sulfate, filtered and concentrated to give a crude product. The crude product was purified by column chromatography (methanol/dichloromethane=0%-10%) to give compound 39-2. MS m/z:455.0, 456.9 [M+1]⁺.

### Step 2: Synthesis of Compound 39-3

4,5-bis(diphenylphosphino)-9,9-dimethylxanthene (343.10 mg, 592.96 μmol) and cesium carbonate (1.93 g, 5.93 mmol) were added to a solution of compound 39-2 (1.35 g, 2.96 mmol) in dioxane (10 mL). The reaction system was purged with nitrogen three times, and palladium acetate (66.56 mg, 296.48 μmol) was added. The resulting mixture was reacted at 110° C. under nitrogen for 2 hours. The reaction solution was concentrated to give a crude product. The crude product was purified by column chromatography (methanol/dichloromethane=1%-10%) to give compound 39-3. MS m/z: 375.0 [M+1]⁺.

### Step 3: Synthesis of a Hydrochloride of Compound 39-4

Compound 39-3 (0.87 g, 2.32 mmol) was added to hydrochloric acid/ethyl acetate (4 M, 8.70 mL), and the resulting mixture was reacted at 25° C. for 2 hours. The reaction system was concentrated to give a hydrochloride of compound 39-4. MS m/z: 275.3 [M+1]⁺.

### Step 4: Synthesis of Compound 39-5

The hydrochloride of compound 39-4 (0.4 g, 1.29 mmol) was dissolved in DCM (4 mL). A 37% aqueous solution of formaldehyde (1.04 g, 12.87 mmol, 958.38 μL) and acetic acid (772.90 mg, 12.87 mmol, 736.09 μL) were added, and the mixture was reacted at 25° C. for 0.5 hours. NaBH(OAc)₃ (545.57 mg, 2.57 mmol) was added, and the mixture was reacted at 25° C. for 0.5 hours. Water (5 mL), dichloromethane (5 mL), and aqueous ammonia (5 mL) were added, and the layers were separated. The organic phase was dried over anhydrous sodium sulfate, filtered, and concentrated. The resulting crude product was purified by column chromatography (methanol/dichloromethane=10%) to give compound 39-5. MS m/z: 289.4 [M+1]⁺.

### Step 5: Synthesis of Compound 39-6

Compound 39-5 (50 mg, 173.40 μmol) was added to methanol (1 mL) and water (0.25 mL). Sodium hydroxide (69.36 mg, 1.73 mmol) was added, and the mixture was reacted at 80° C. for 12 hours. The reaction system was concentrated, and water (2 mL) was added. The aqueous phase was extracted with a mixture of dichloromethane and methanol (10:1, 10 mL). The organic phase was dried over anhydrous sodium sulfate, filtered, and concentrated under reduced pressure to give compound 39-6. MS m/z: 221.4 [M+1]⁺.

### Step 6: Synthesis of Compound 39-7

Compound 30-6 (50 mg, 125.36 μmol) was added to dioxane (1 mL) under nitrogen. Compound 39-6 (30.38 mg, 137.90 μmol), methanesulfonato[9,9-dimethyl-4,5-bis(diphenylphosphino)xanthene](2′-methylamino-1,1′-biphenyl-2-yl)palladium(II) (12.06 mg, 12.54 μmol), and cesium carbonate (81.69 mg, 250.73 μmol) were added. The resulting mixture was reacted at 110° C. for 2 hours. Water (5 mL) was added, and the mixture was extracted with ethyl acetate (5 mL). The organic phase was dried over anhydrous sodium sulfate, filtered and concentrated. The resulting crude product was purified by column chromatography (methanol/dichloromethane=5%-10%) to give compound 39-7. MS m/z: 583.1 [M+1]⁺.

### Step 7: Synthesis of a Trifluoroacetate of Compound 39

Compound 39-7 (36 mg, 61.79 μmol) was dissolved in DCM (1 mL) under nitrogen, and trifluoroacetic acid (719.98 μL) was added. The resulting mixture was reacted at 25° C. for 1 hour. The reaction system was concentrated under reduced pressure to give a crude product. The crude product was purified by prep-HPLC (chromatography column: Phenomenex luna C18 100*40 mm*3 μm; mobile phase: [water (TFA)-acetonitrile]; acetonitrile %: 1%-35%, 8 min) to give a trifluoroacetate of compound 39. MS m/z: 483.5 [M+1]⁺; ¹H NMR (400 MHz, DMSO-d₆) δ 9.88 (s, 1H), 9.52 (s, 1H), 9.18 (s, 1H), 8.36 (d, J=5.0 Hz, 1H), 7.57 (d, J=3.4 Hz, 1H), 7.46 (d, J=8.8 Hz, 1H), 7.38 (d, J=5.0 Hz, 1H), 6.91 (d, J=3.6 Hz, 1H), 6.77 (d, J=8.4 Hz, 1H), 4.72 (s, 2H), 4.53-4.47 (m, 1H), 4.21-4.17 (m, 1H), 4.11-4.04 (m, 1H), 3.88 (s, 3H), 3.61-3.55 (m, 2H), 3.21-3.08 (m, 2H), 3.03-2.92 (m, 2H), 2.89 (s, 3H).

### Example 41

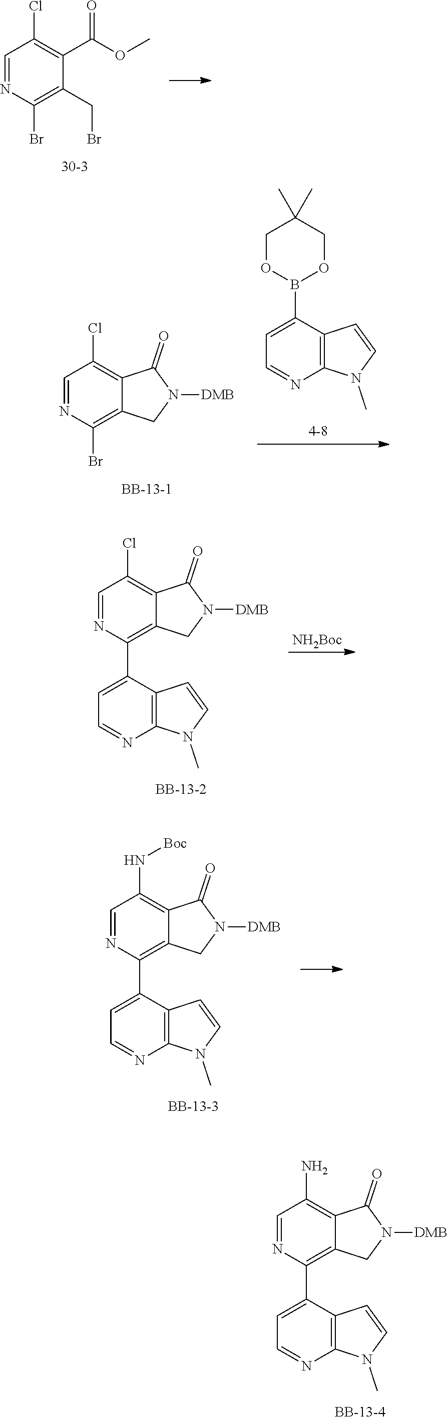

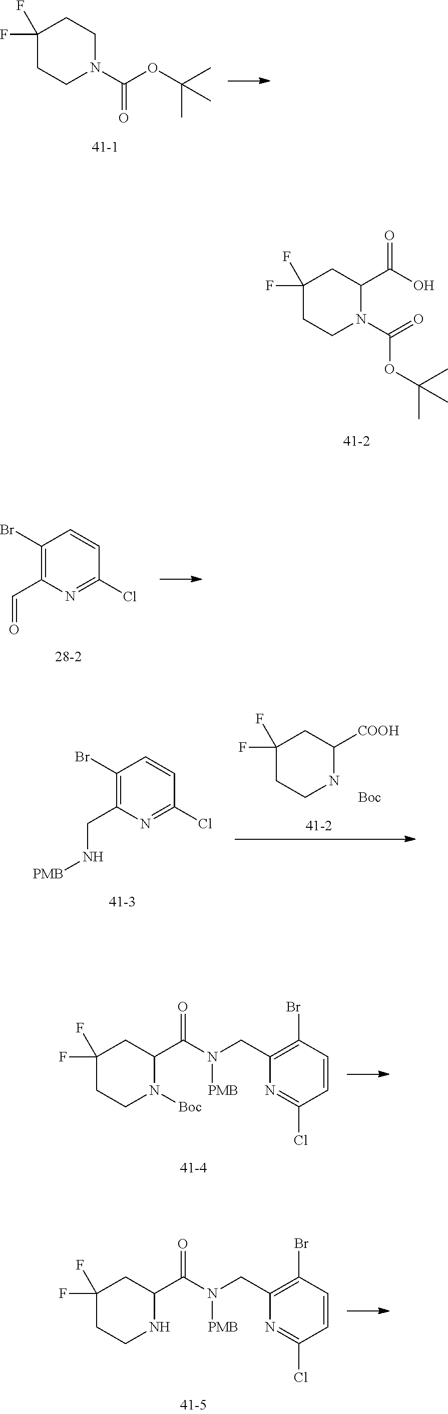

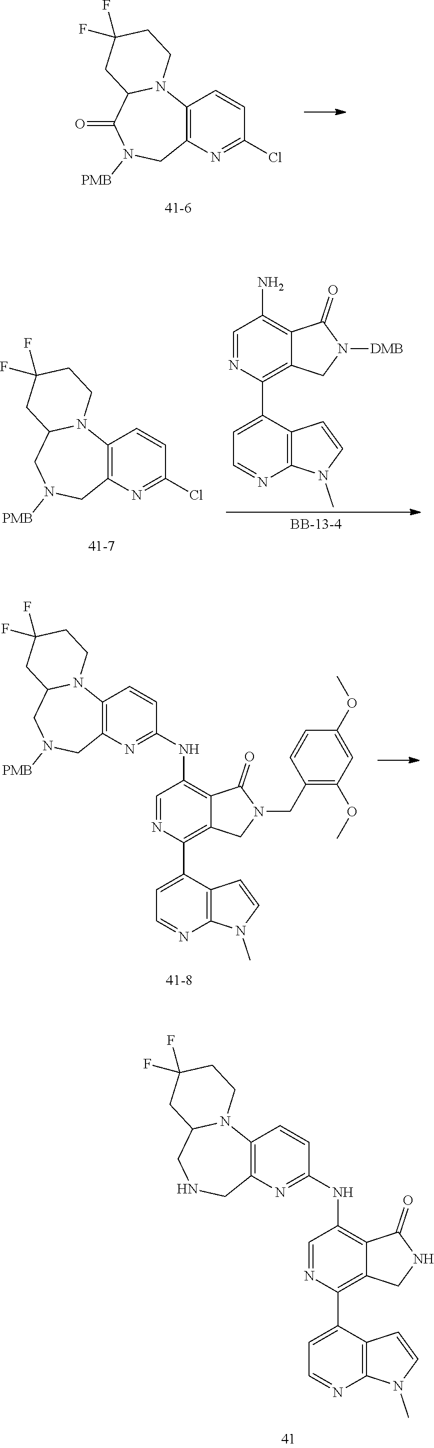

### Step 1: Synthesis of Compound BB-13-1

Compound 30-3 (8.9 g, 25.92 mmol) was dissolved in tetrahydrofuran (100 mL) in a bottle. 2,4-dimethoxybenzylamine (8.67 g, 51.83 mmol) was added dropwise at 0° C. The reaction solution was warmed to 25° C., stirred for 5 hours, and directly concentrated to give a crude product. The crude product was added to methyl tert-butyl ether, ethyl acetate, and ethanol (50 mL of each), and stirred for 16 hours. The mixture was filtered, and the filter cake was dried to give compound BB-13-1. MS m/z: 397.0, 399.0 [M+1, M+3]⁺.

### Step 2: Synthesis of Compound BB-13-2

Compound 4-8 (2.52 g, 10.31 mmol) and sodium carbonate (2.19 g, 20.62 mmol) were added to a solution of compound BB-13-1 (4.1 g, 10.31 mmol) in dioxane (80 mL) and water (16 mL). The reaction system was purged with nitrogen three times. Pd(PPh₃)₄ (1.19 g, 1.03 mmol) was added, and the mixture was reacted at 100° C. under nitrogen for 2 hours. The mixture was cooled to room temperature, and vacuum filtered. The residue was washed with dichloromethane (20 mL). The filtrate was concentrated under reduced pressure to give a crude product. Methanol (50 mL) was added, and the mixture was stirred and vacuum filtered. The filter cake was washed with methanol (20 mL) to give BB-13-2. MS m/z: 449.1 [M+1]⁺.

### Step 3: Synthesis of Compound BB-13-3

BocNH₂ (153.97 mg, 1.31 mmol), Pd₂(dba)₃ (60.18 mg, 65.72 μmol), and RuPhos (61.33 mg, 131.43 μmol) were added to a solution of compound BB-13-2 (295 mg, 657.16 μmol) in dioxane (5 mL). The reaction system was purged with nitrogen three times, and potassium phosphate (418.48 mg, 1.97 mmol) was added. The resulting mixture was reacted at 110° C. under nitrogen for 2 hours. The mixture was cooled to room temperature and vacuum filtered. The residue was washed with dichloromethane (10 mL). The filtrate was concentrated under reduced pressure. The resulting crude product was purified by column chromatography (mobile phase: 0-70% EA/PE) to give compound BB-13-3. MS m/z: 530.1 [M+1]⁺.

### Step 4: Synthesis of Compound BB-13-4

TFA (7.70 g, 67.53 mmol) was added to compound BB-13-3 (463 mg, 874.27 mol), and the mixture was reacted at 25° C. for 16 hours. The mixture was concentrated under reduced pressure to give a crude product. Dichloromethane (10 mL) was added, and pH of the mixture was adjusted to be greater than 10 with a saturated sodium carbonate solution (20 mL). The mixture was extracted with dichloromethane (10 mL×3). The organic phases were combined, dried over anhydrous sodium sulfate, and filtered. The filtrate was concentrated under reduced pressure to give a crude product. The crude product was purified by column chromatography (0-100% EA/PE) to give compound BB-13-4. MS m/z: 430.1 [M+1]⁺.

### Step 5: Synthesis of Compound 41-2

Compound 41-1 (10 g, 45.20 mmol) was dissolved in THF (300 mL) in a dry vial. s-BuLi (1.3 M, 62.58 mL) was slowly added dropwise at −30° C. under nitrogen. After the addition was completed, the reaction solution was stirred at −30° C. for 30 minutes. Then, carbon dioxide gas was slowly introduced at −30° C. Then, the reaction solution was slowly warmed to 25° C., and stirred for another 16 hours. The reaction solution was poured into water (200 mL), and the layers were separated. The aqueous phase was washed with methyl tert-butyl ether (100 mL×2). Then, the pH of the aqueous phase was adjusted to 3 to 4 with 0.5N dilute hydrochloric acid. Then, the aqueous phase was extracted with ethyl acetate (100 mL×3). The organic phases were combined, dried over anhydrous sodium sulfate, filtered and concentrated. The crude product was purified by column chromatography (petroleum ether:ethyl acetate (v/v)=10:1 to 2:1) to give compound 41-2. ¹H NMR (400 MHz, CDCl₃) δ 5.15-4.93 (m, 1H), 4.19-4.07 (m, 1H), 3.34-3.23 (m, 1H), 2.80-2.71 (m, 1H), 2.23-1.78 (m, 3H), 1.49-1.45 (m, 9H).

### Step 6: Synthesis of Compound 41-3

Compound 28-2 (2.6 g, 11.79 mmol) and p-methoxybenzylamine (1.78 g, 12.97 mmol) were dissolved in DCM (50 mL) in a dry vial, and AcOH (708.23 mg, 11.79 mmol) was added. The reaction solution was stirred at 25° C. for 1 hour. Then, NaBH(OAc)₃ (3.0 g, 14.15 mmol) was added. The reaction solution was stirred at 25° C. for another 2 hours. 50 mL of saturated sodium carbonate solution was added to the reaction solution. The resulting mixture was stirred evenly, and the pH of the system was tested to be greater than 8. The layers were separated. The aqueous phase was extracted with DCM (20 mL×2). The organic phases were combined, dried over anhydrous sodium sulfate, filtered, and concentrated. The crude product was purified by column chromatography (petroleum ether:ethyl acetate (v/v)=10:1-1:1) to give compound 41-3. MS m/z: 340.8 [M+1]⁺; ¹H NMR (400 MHz, CDCl₃) δ=7.75 (d, J=8.4 Hz, 1H), 7.31-7.27 (m, 2H), 7.10 (d, J=8.4 Hz, 1H), 6.89-6.86 (m, 2H), 3.99 (s, 2H), 3.81 (s, 5H).

### Step 7: Synthesis of Compound 41-4

Compound 41-3 (1.02 g, 2.99 mmol) and compound 41-2 (793 mg, 2.99 mmol) were dissolved in DMF (30 mL) in a dry vial. DIEA (772.77 mg, 5.98 mmol), HOBt (807.93 mg, 5.98 mmol) and HATU (2.27 g, 5.98 mmol) were added. The reaction solution was stirred and reacted at 25° C. for 16 hours. 1M dilute hydrochloric acid solution (10 mL) was added to the solution to adjust the pH to 6. The concentrate was extracted with added dichloromethane (50 mL×3). The organic phases were combined, dried over anhydrous sodium sulfate, filtered and concentrated. The crude product was separated by column chromatography (dichloromethane:methanol (v/v)=100:0 to 90:10) to give compound 41-4. MS m/z: 588.0 [M+1]⁺; ¹H NMR (400 MHz, CDCl₃) δ 7.78-7.73 (m, 1H), 7.25-7.21 (m, 1H), 7.20-7.04 (m, 2H), 6.92-6.88 (m, 1H), 6.85-6.73 (m, 1H), 5.02-4.50 (m, 5H), 3.99-3.95 (m, 1H), 3.85-3.71 (m, 4H), 2.74-2.57 (m, 1H), 2.49-2.07 (m, 2H), 2.03-1.85 (m, 1H), 1.48-1.38 (m, 9H).

### Step 8: Synthesis of Compound 41-5

ZnBr₂ (1.91 g, 8.49 mmol) was added to a flask containing a solution of compound 41-4 (1 g, 1.70 mmol) in DCM (30 mL). The mixture was stirred and reacted at 25° C. for 16 hours. The reaction solution was concentrated. 20 mL of ethyl acetate and 20 mL of water were added, and the mixture was stirred. The layers were separated. The mixture was extracted with ethyl acetate (15 mL×3). The organic phases were combined, dried over anhydrous sodium sulfate, concentrated, and separated by column chromatography (petroleum ether:ethyl acetate=100:0 to 30:70) to give compound 41-5. MS m/z:487.9 [M+1]⁺; ¹H NMR (400 MHz, DMSO-d₆) δ=7.21 (d, J=8.8 Hz, 1H), 7.12 (d, J=8.8 Hz, 2H), 6.92 (d, J=8.4 Hz, 1H), 6.83 (d, J=8.8 Hz, 2H), 5.07-4.87 (m, 2H), 3.74 (s, 2H), 3.71 (s, 3H), 3.57-3.51 (m, 1H), 3.11-2.99 (m, 2H), 1.92-1.78 (m, 4H).

### Step 9: Synthesis of Compound 41-6

Cs₂CO₃ (133.33 mg, 409.20 μmol) was added to a flask containing a solution of compound 41-5 (100 mg, 204.60 μmol) in 1,4-dioxane (5 mL). The reaction system was tested to be alkaline. BINAP (50.96 mg, 81.84 μmol) and Pd(OAc)₂ (9.19 mg, 40.92 μmol) were added under nitrogen. The reaction solution was stirred and reacted at 110° C. under nitrogen for 16 hours. The mixture was concentrated. 10 mL of ethyl acetate and 10 mL of water were added, and the mixture was stirred. The layers were separated. The aqueous phase was extracted with ethyl acetate (10 mL×3). The organic phases were combined, dried and concentrated to give a crude product. The crude product was purified by column chromatography (petroleum ether:ethyl acetate (v/v)=100:0 to 60:40) to give compound 41-6. MS m/z:407.9 [M+1]⁺.

### Step 10: Synthesis of Compound 41-7

Compound 41-6 (70 mg, 171.64 μmol) was dissolved in THF (3 mL) in a round-bottom flask. BH₃·THF (1 M, 1.72 mL) was added at 0° C. under nitrogen. The reaction solution was gradually heated to 60° C., stirred and reacted for 16 hours under nitrogen. Methanol (10 mL) was added to the reaction solution under nitrogen to quench the reaction. The mixture was concentrated under reduced pressure to give a crude product. The crude product was separated by column chromatography (petroleum ether:ethyl acetate (v/v)=100:0 to 70:30) to give compound 41-7. MS m/z: 394.0 [M+1]⁺.

### Step 11: Synthesis of Compound 41-8

Cs₂CO₃ (9.93 mg, 30.48 μmol) was added to a round-bottom flask containing a solution of compound 41-7 (4 mg, 10.16 μmol) and compound BB-13-4 (4.36 mg, 10.16 μmol) in 1,4-dioxane (2 mL). The reaction system was tested to be alkaline, and Xantphos Pd G₄ (1.95 mg, 2.03 μmol) was added under nitrogen. The reaction solution was stirred and reacted at 110° C. under nitrogen for 5 hours. The crude product was purified by a preparative chromatography plate (dichloromethane:methanol=10:1, Rf=0.4) to give compound 41-8. MS m/z: 787.5 [M+1]⁺.

### Step 12: Synthesis of a Trifluoroacetate of Compound 41

TfOH (30.52 mg, 203.34 μmol) was added to a flask containing a solution of compound 41-8 (8.00 mg, 10.17 μmol) in DCM (1 mL). The reaction solution was stirred and reacted at 25° C. for 16 hours. The reaction solution was concentrated, and aqueous ammonia (2 mL) was added. The pH of the reaction system was tested to be 9.1 mL of DMF was added to the solution. The resulting mixture was purified by prep-HPLC (chromatography column: Phenomenex Luna C18 75*30 mm*3 μm; mobile phase: [water (TFA)-acetonitrile; acetonitrile %: 5%-35%, 8 min] to give a trifluoroacetate of compound 41. MS m/z: 517.2 [M+1]⁺; 1H NMR (400 MHz, DMSO-d₆) δ 10.04 (s, 1H), 9.72 (s, 1H), 9.22-9.06 (m, 2H), 8.36-8.35 (m, 1H), 7.61-7.58 (m, 2H), 7.38 (s, 1H), 7.12-7.09 (m, 1H), 6.95-6.89 (m, 1H), 4.73 (s, 2H), 44.55-4.38 (m, 2H), 3.87 (s, 3H), 3.45-3.42 (m, 3H), 3.26-3.22 (m, 3H), 2.22-2.10 (m, 3H).

### Example 42

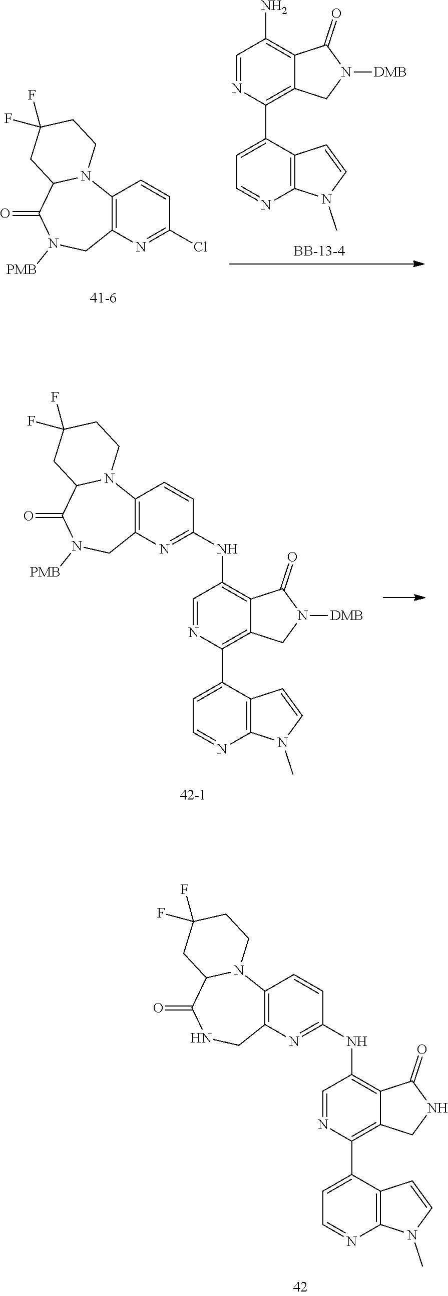

### Step 1: Synthesis of Compound 42-1

Cs₂CO₃ (71.90 mg, 220.67 μmol), BINAP (13.74 mg, 22.07 μmol) and Pd(OAc)₂ (3.30 mg, 14.71 μmol) were added to a solution of compound 41-6 (30 mg, 73.56 μmol) and compound BB-13-4 (29.24 mg, 73.56 μmol) in 1,4-dioxane (5 mL). The reaction solution was stirred at 110° C. under nitrogen for 16 hours. The mixture was filtered, and the filter cake was washed with ethyl acetate (20 mL×2). The filtrate was concentrated to give a crude product. The crude product was purified by a preparative chromatography plate (dichloromethane:methanol 20:1) to give compound 42-1. MS m/z: 801.2 [M+1]⁺.

### Step 2: Synthesis of a Trifluoroacetate of Compound 42

TfOH (488.00 mg, 3.25 mmol) was added to a flask containing a solution of compound 42-1 (50 mg, 65.03 μmol) in DCM (2 mL), and the reaction solution was stirred at 25° C. for 16 hours. Aqueous ammonia was added to adjust the pH to 8 to 9, and the reaction solution was concentrated under reduced pressure. The crude product was purified by prep-HPLC (chromatography column: Phenomenex Luna C18 75*30 mm*3 μm; mobile phase: [water (TFA)-acetonitrile]; acetonitrile %: 20%-50%, 8 min) to give a trifluoroacetate of compound 42. MS m/z: 531.2 [M+1]⁺; ¹H NMR (400 MHz, DMSO-d₆) δ 10.01 (s, 1H), 9.67 (s, 1H), 9.19 (s, 1H), 8.49 (s, 1H), 8.35-8.34 (m, 1H), 7.61-7.58 (m, 2H), 7.38 (s, 1H), 7.12-7.09 (m, 1H), 6.90 (s, 1H), 4.77-4.71 (m, 3H), 4.01-3.99 (m, 1H), 3.87 (s, 3H), 3.53-3.48 (m, 2H), 3.23-3.16 (m, 1H), 2.38-2.34 (m, 2H), 2.32-2.22 (m, 2H).

### Example 43

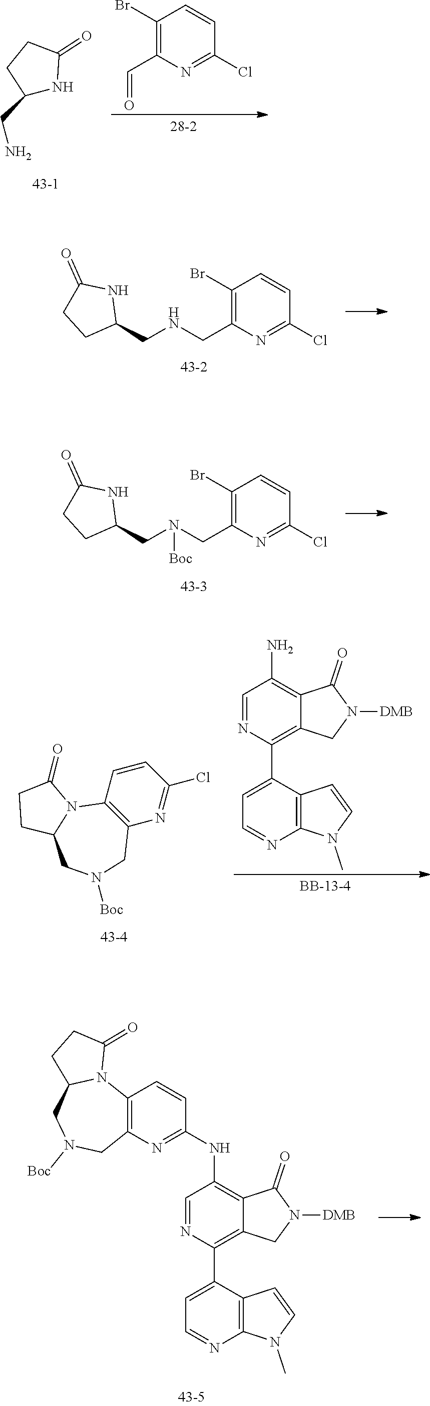

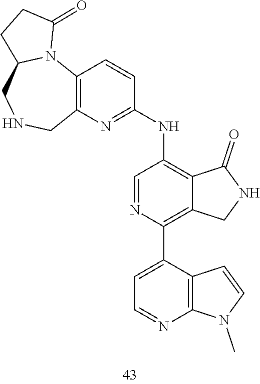 [SMILES: Cn1ccc2c(-c3ncc(Nc4ccc5c(n4)CNC[C@H]4CCC(=O)N54)c4c3CNC4=O)ccnc21]

### Step 1: Synthesis of Compound 43-2

Compound 43-1 (248.53 mg, 2.18 mmol) was dissolved in methanol (10 mL) under nitrogen. Compound 28-2 (400 mg, 1.81 mmol) and acetic acid (108.96 mg, 1.81 mmol) were added, and the resulting mixture was reacted at 20° C. for 1 hour. Sodium acetate borohydride (576.84 mg, 2.72 mmol) was added, and the resulting mixture was reacted at 20° C. for 15 hours. The mixture was concentrated under reduced pressure, and a saturated sodium carbonate solution (20 mL) was added to quench the reaction. The mixture was extracted with dichloromethane (20 mL*3). The organic phases were combined, washed with saturated brine (20 mL), dried over anhydrous sodium sulfate, filtered and concentrated to give a crude product. The crude product was separated by column chromatography (methanol/dichloromethane=0%-10%) to give compound 43-2. MS m/z: 318.0, 320.0 [M+1]⁺.

### Step 2: Synthesis of Compound 43-3

Compound 43-2 (223 mg, 699.94 μmol) was dissolved in tetrahydrofuran (5 mL) under nitrogen. Triethylamine (212.48 mg, 2.10 mmol) and Boc₂O (229.14 mg, 1.05 mmol) were added, and the mixture was reacted at 20° C. for 2 hours. A saturated sodium bicarbonate solution (20 mL) was added, and the mixture was extracted with ethyl acetate (20 mL×3). The organic phases were combined, washed with saturated brine (20 mL), dried over anhydrous sodium sulfate, filtered, and concentrated under reduced pressure to give a crude product. The crude product was purified by column chromatography (ethyl acetate/petroleum ether=0%-50%) to give compound 43-3. MS m/z: 440.0, 442.0 [M+Na]⁺.

### Step 3: Synthesis of Compound 43-4

Compound 43-3 (229 mg, 546.91 μmol) was dissolved in 1,4-dioxane (8 mL) under nitrogen. N,N-dimethylethylenediamine (9.64 mg, 109.38 μmol), cesium carbonate (534.59 mg, 1.64 mmol), and copper(I) iodide (10.42 mg, 54.69 μmol) were added. The mixture was reacted at 110° C. for 20 hours. The mixture was cooled to room temperature and filtered. The filter cake was washed with dichloromethane (10 mL). The filtrate was concentrated under reduced pressure to give a crude product. The crude product was purified by column chromatography (ethyl acetate/dichloromethane=0%-40%) to give compound 43-4. MS m/z: 360.2 [M+Na]⁺; ¹H NMR (400 MHz, CDCl₃) δ 7.80 (d, J=8.4 Hz, 1H), 7.30 (d, J=8.0 Hz, 1H), 4.88 (d, J=16.0 Hz, 1H), 4.30-4.06 (m, 2H), 3.93-3.81 (m, 1H), 3.22-3.08 (m, 1H), 2.77-2.50 (m, 2H), 2.48-2.32 (m, 1H), 1.97-1.82 (m, 1H), 1.39 (s, 9H).

### Step 4: Synthesis of Compound 43-5

Compound BB-13-4 (100 mg, 232.85 μmol) was dissolved in 1,4-dioxane (4 mL) under nitrogen. Compound 43-4 (86.52 mg, 256.13 μmol), cesium carbonate (227.60 mg, 698.54 mol), Ruphos (21.73 mg, 46.57 μmol), and Pd₂(dba)₃ (21.32 mg, 23.28 μmol) were added. The mixture was reacted at 110° C. for 2 hours. The mixture was cooled to room temperature and filtered. The filter cake was washed with dichloromethane (20 mL). The filtrate was concentrated under reduced pressure to give a crude product. The crude product was purified by column chromatography (ethyl acetate/petroleum ether=0%-60%) to give compound 43-5. MS m/z: 731.3 [M+1]⁺; ¹H NMR (400 MHz, CDCl₃) δ 10.46 (s, 1H), 9.85 (s, 1H), 8.41 (d, J=5.2 Hz, 1H), 7.71 (d, J=8.4 Hz, 1H), 7.47-7.38 (m, 1H), 7.13-7.01 (m, 1H), 6.88 (d, J=8.4 Hz, 2H), 6.53-6.41 (m, 2H), 5.02 (d, J=15.6 Hz, 1H), 4.76 (s, 2H), 4.59-4.49 (m, 1H), 4.31-4.17 (m, 2H), 4.07-3.90 (m, 3H), 3.84 (s, 5H), 3.80 (s, 3H), 3.17-3.03 (m, 1H), 2.74-2.53 (m, 3H), 2.52-2.32 (m, 2H), 1.42 (s, 9H).

### Step 5: Synthesis of a Trifluoroacetate of Compound 43

Compound 43-5 (126 mg, 172.41 μmol) was dissolved in TfOH (22.60 mmol, 2 mL) under nitrogen, and the mixture was reacted at 40° C. for 16 hours. The system was slowly poured into dilute aqueous ammonia (5% concentration) (10 mL). The mixture was extracted with dichloromethane:methanol=10:1 (V/V, 20 mL×5). The organic phases were combined, dried over anhydrous sodium sulfate, and filtered. The filtrate was concentrated to give a crude product. The crude product was purified by prep-HPLC (column: Phenomenex luna C18 100*40 mm*3 μm; mobile phase: [A: aqueous phase (0.1% TFA); B: acetonitrile]; B %: 1.00%-30.00%, 8.00 min) to give a trifluoroacetate of compound 43. MS m/z: 481.2 [M+H]⁺; ¹H NMR (400 MHz, DMSO-d₆) δ 10.12 (s, 1H), 9.96 (s, 1H), 9.30 (s, 1H), 8.37 (d, J=4.8 Hz, 1H), 7.80 (d, J=8.8 Hz, 1H), 7.59 (d, J=3.6 Hz, 1H), 7.41 (d, J=4.8 Hz, 1H), 7.25 (d, J=8.8 Hz, 1H), 6.90 (d, J=3.6 Hz, 1H), 4.74 (s, 2H), 4.51-4.47 (m, 3H), 4.00-3.82 (m, 5H), 2.43-2.40 (m, 4H), 1.86-1.84 (m, 1H). The trifluoroacetate of compound 43 was added to a sodium bicarbonate solution, and the mixture was extracted with ethyl acetate. The organic phase was dried over anhydrous sodium sulfate and concentrated under reduced pressure to give compound 43.

### Example 44

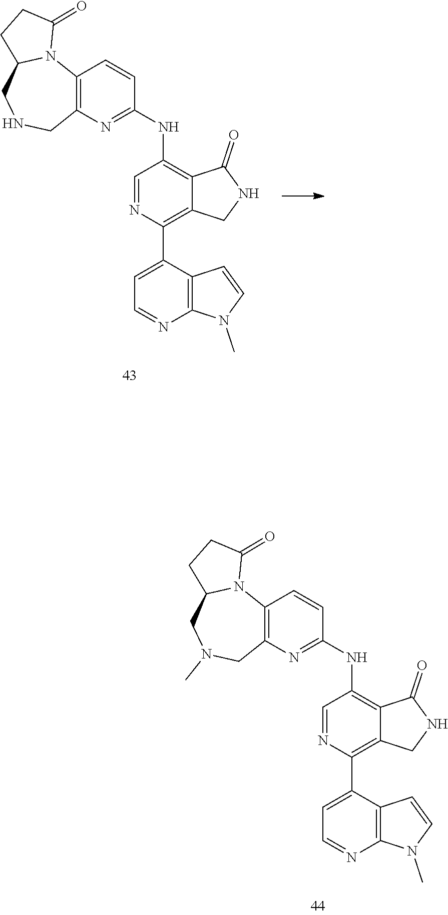 [SMILES: Cn1ccc2c(-c3ncc(Nc4ccc5c(n4)CNC[C@H]4CCC(=O)N54)c4c3CNC4=O)ccnc21 ; CN1Cc2nc(Nc3cnc(-c4ccnc5c4ccn5C)c4c3C(=O)NC4)ccc2N2C(=O)CC[C@@H]2C1]

### Step 1: Synthesis of a Trifluoroacetate of Compound 44

Compound 43 (30 mg, 62.43 μmol) was dissolved in MeOH (2 mL) in a round-bottom flask. 37% aqueous solution of formaldehyde (25.34 mg, 312.16 μmol, 23.24 μL) and acetic acid (3.75 mg, 62.43 μmol) were added. The mixture was stirred at 15° C. for 1 hour. Then, NaBH(OAc)₃ (26.46 mg, 124.86 μmol) was added, and the mixture was stirred at 15° C. for another 16 hours. The reaction solution was directly filtered, and the filtrate was concentrated. The crude product was purified by prep-HPLC (column: Phenomenex luna C18 100*40 mm*3 m; mobile phase: [A: aqueous phase (0.1% TFA); B: acetonitrile]; B %: 1.00%-30.00%, 8.00 min) to give a trifluoroacetate of compound 44. MS m/z: 495.2 [M+H]⁺; ¹H NMR (400 MHz, DMSO-d₆) δ 10.37-9.96 (m, 2H), 9.29 (s, 1H), 8.37 (d, J=4.8 Hz, 1H), 7.81-7.80 (m, 1H), 7.60 (d, J=7.6 Hz, 1H), 7.41 (d, J=4.8 Hz, 1H), 7.27 (d, J=8.8 Hz, 1H), 6.90 (d, J=3.6 Hz, 1H), 4.78-4.59 (m, 4H), 3.97-3.88 (m, 4H), 3.65-3.54 (m, 2H), 3.39-3.12 (m, 3H), 2.67 (br s, 1H), 2.46-2.33 (m, 2H), 1.83-1.80 (m, 1H).

### Example 45

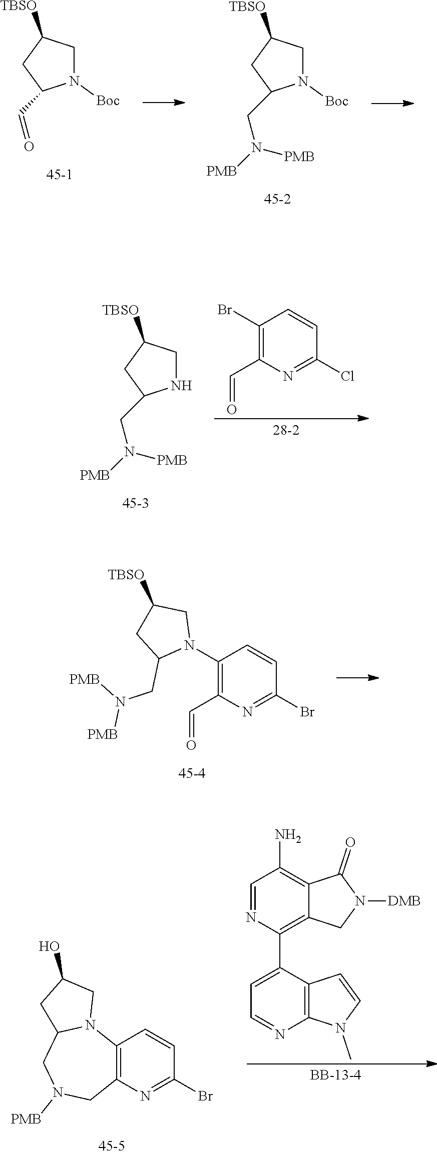

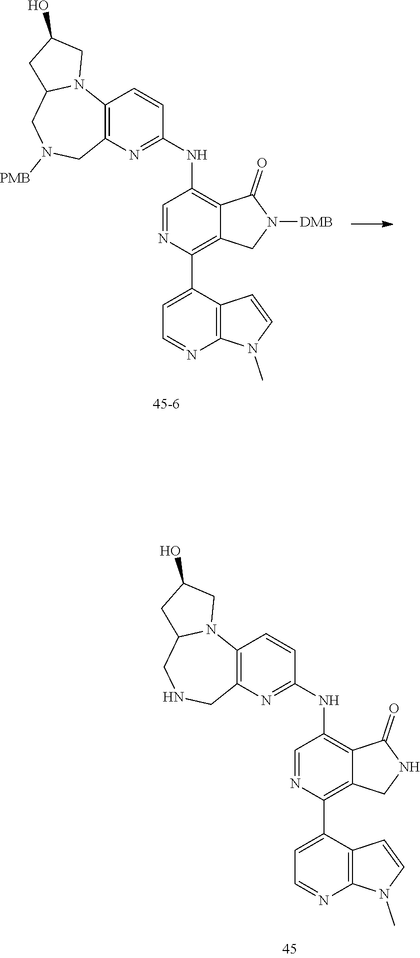

### Step 1: Synthesis of Compound 45-2

Compound 45-1 (6.1 g, 18.51 mmol) was dissolved in DCM (61 mL) in a round-bottom flask. The reaction system was purged with nitrogen three times. Bis(4-methoxybenzyl)amine (4.76 g, 18.51 mmol) and acetic acid (1.11 g, 18.51 mmol) were added. The mixture was reacted at 25° C. for 0.5 hours. NaBH(OAc)₃ (7.85 g, 37.03 mmol) was added, and the mixture was reacted for another 0.5 hours. Water (50 mL) was added, and the layers were separated. The organic phase was washed with saturated sodium bicarbonate (50 mL×2). The organic phases were combined, dried over anhydrous sodium sulfate, filtered and concentrated to give a crude product. The crude product was purified by column chromatography (petroleum ether:ethyl acetate=3:1) to give compound 45-2. MS m/z: 571.3 [M+1]⁺; ¹H NMR (400 MHz, CDCl₃) δ 7.29-7.26 (m, 4H), 6.88-6.82 (m, 4H), 4.11-3.90 (m, 3H), 3.81 (s, 6H), 3.76-3.75 (m, 2H), 3.44-3.10 (m, 4H), 2.68-2.48 (m, 1H), 1.93-1.76 (m, 2H), 1.47 (s, 9H), 0.85 (s, 9H), 0.00 (s, 6H).

### Step 2: Synthesis of Compound 45-3

Compound 45-2 (2 g, 3.50 mmol) was dissolved in DCM (50 mL) in a round-bottom flask. ZnBr₂ (3.95 g, 17.52 mmol) was added, and the reaction solution was stirred at 25° C. for 16 hours. The reaction solution was poured into water (100 mL), and the layers were separated. The aqueous phase was extracted with DCM (30 mL×2). The organic phases were combined, dried over anhydrous sodium sulfate, filtered and concentrated to give a crude product. The crude product was purified by column chromatography (petroleum ether:ethyl acetate=5:1-0:1) to give compound 45-3. MS m/z: 471.1 [M+1]⁺.

### Step 3: Synthesis of Compound 45-4

Compound 45-3 (400 mg, 849.77 μmol) was dissolved in DMF (5 mL) under nitrogen. Compound 28-2 (190.68 mg, 934.74 μmol) and DIEA (329.48 mg, 2.55 mmol) were added. The mixture was reacted at 100° C. for 1 hour. The mixture was cooled, and water (20 mL) was added to quench the reaction. The mixture was extracted with ethyl acetate (20 mL×3). The organic phases were combined, washed with saturated brine (10 mL), dried over anhydrous sodium sulfate, and filtered. The filtrate was concentrated under reduced pressure. The resulting crude product was separated by column chromatography (ethyl acetate/petroleum ether=0%-15%) to give compound 45-4. MS m/z: 654.2, 656.1 [M+1]⁺.

### Step 4: Synthesis of Compound 45-5

Compound 45-4 (140 mg, 213.84 μmol) was dissolved in TFA (67.53 mmol, 5 mL) under nitrogen, and the mixture was reacted at 60° C. for 16 hours. The mixture was cooled and concentrated under reduced pressure. DCM (1 mL) and NaBH(OAc)₃ (135.96 mg, 641.51 mol) were added, and the mixture was reacted at 25° C. for 1 hour. A saturated sodium carbonate solution (10 mL) was added to quench the reaction, and the mixture was extracted with dichloromethane (20 mL×3). The organic phases were combined, washed with saturated brine (20 mL), dried over anhydrous sodium sulfate, filtered and concentrated under reduced pressure to give a crude product. The crude product was separated by column chromatography (ethyl acetate/petroleum ether=0%-100%) to give compound 45-5. MS m/z: 404.1, 406.1 [M+1]⁺; ¹H NMR (400 MHz, DMSO-d₆) δ 7.30 (d, J=8.4 Hz, 1H), 7.21 (d, J=8.4 Hz, 2H), 7.04 (d, J=8.8 Hz, 1H), 6.88 (d, J=8.8 Hz, 2H), 5.08 (d, J=4.0 Hz, 1H), 4.33-4.24 (m, 1H), 4.07 (d, J=14.4 Hz, 1H), 3.74 (s, 4H), 3.72-3.64 (m, 1H), 3.56 (s, 2H), 3.50 (d, J=14.4 Hz, 1H), 3.10-3.04 (m, 1H), 2.90-2.87 (m, 1H), 1.93-1.87 (m, 1H), 1.66-1.63 (m, 1H).

### Step 5: Synthesis of Compound 45-6

Compound BB-13-4 (75 mg, 174.63 μmol) was dissolved in 1,4-dioxane (5 mL) under nitrogen. Compound 45-5 (70.60 mg, 174.63 μmol), cesium carbonate (170.70 mg, 523.90 μmol), and Xantphos Pd G₄ (16.81 mg, 17.46 μmol) were added. The mixture was reacted at 110° C. for 2 hours. The mixture was cooled, filtered, and washed with dichloromethane (10 mL). The filtrate was concentrated under reduced pressure. The resulting crude product was separated by column chromatography (methanol/ethyl acetate=0%-10%) to give compound 45-6. MS m/z: 753.3 [M+Na]⁺.

### Step 6: Synthesis of a Trifluoroacetate of Compound 45

Compound 45-6 (85 mg, 112.90 μmol) was dissolved in TfOH (45.20 mmol, 4 mL) under nitrogen, and the mixture was reacted at 40° C. for 5 hours. The mixture was cooled to room temperature, and the system was slowly added into dilute aqueous ammonia (10 mL). The mixture was extracted with dichloromethane:methanol=10:1 (20 mL×4). The organic phases were combined, dried over anhydrous sodium sulfate, filtered, and concentrated to give a crude product. The crude product was purified by prep-HPLC (column: Phenomenex luna C18 80*30 mm*3 μm; mobile phase: [A: aqueous phase (0.1% TFA); B: acetonitrile]; acetonitrile %: 1%-20%, 8.00 min) to give a trifluoroacetate of compound 45. MS m/z: 483.2 [M+1]⁺; ¹H NMR (400 MHz, DMSO-d₆) δ 9.99 (s, 1H), 9.63 (s, 1H), 9.19 (s, 1H), 8.36 (d, J=4.8 Hz, 1H), 7.57 (d, J=3.6 Hz, 1H), 7.44 (d, J=8.8 Hz, 1H), 7.38 (d, J=4.8 Hz, 1H), 7.14 (d, J=8.8 Hz, 1H), 6.89 (d, J=3.6 Hz, 1H), 4.71 (s, 3H), 4.43-4.34 (m, 1H), 4.25-4.16 (m, 1H), 3.87 (s, 3H), 3.37-3.32 (m, 4H), 3.31-3.22 (m, 2H), 3.14-3.05 (m, 1H), 2.11-2.00 (m, 1H), 1.90-1.87 (m, 1H). The trifluoroacetate of compound 45 was added to a sodium bicarbonate solution, and the mixture was extracted with ethyl acetate. The organic phase was dried over anhydrous sodium sulfate and concentrated under reduced pressure to give compound 45.

### Example 46

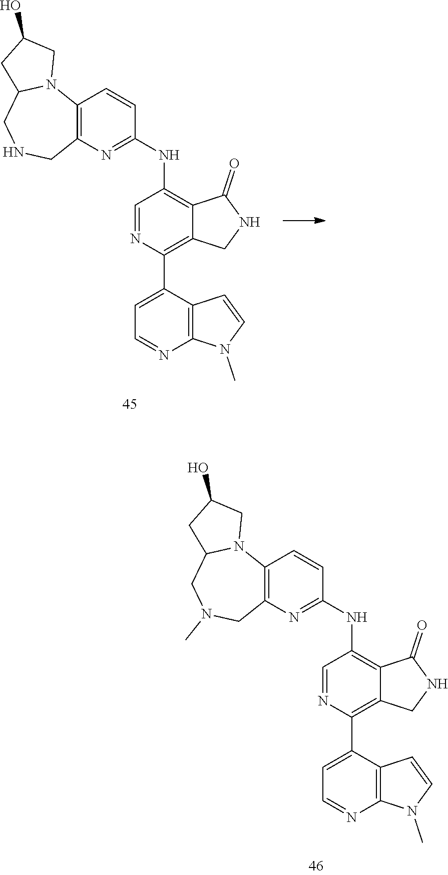 [SMILES: Cn1ccc2c(-c3ncc(Nc4ccc5c(n4)CNCC4C[C@@H](O)CN54)c4c3CNC4=O)ccnc21 ; CN1Cc2nc(Nc3cnc(-c4ccnc5c4ccn5C)c4c3C(=O)NC4)ccc2N2C[C@H](O)CC2C1]

### Step 1: Synthesis of a Trifluoroacetate of Compound 46

Compound 45 (40 mg, 82.90 μmol) was dissolved in methanol (5 mL) under nitrogen. A 37% aqueous solution of formaldehyde (33.64 mg, 414.48 μmol) and acetic acid (14.93 mg, 248.69 μmol) were added, and the mixture was reacted at 25° C. for 1 hour. NaBH(OAc)₃ (35.14 mg, 165.79 μmol) was added, and the mixture was reacted at 25° C. for 1 hour. A saturated sodium carbonate solution (10 mL) was added, and the mixture was extracted with dichloromethane:methanol=10:1 (20 mL×4). The organic phases were combined, dried over anhydrous sodium sulfate, filtered, and concentrated under reduced pressure to give a crude product. The crude product was purified by prep-HPLC (column: Phenomenex luna C18 80*30 mm*3 μm; mobile phase: [A: aqueous phase (0.1% TFA); B: acetonitrile]; B %: 1%-25%, 8.00 min) to give a trifluoroacetate of compound 46. MS m/z: 497.2 [M+1]⁺; ¹H NMR (400 MHz, DMSO-d₆) δ 10.62-10.10 (m, 1H), 10.09-9.90 (m, 1H), 9.80-9.51 (m, 1H), 9.20 (d, J=6.8 Hz, 1H), 8.44-8.25 (m, 1H), 7.58 (d, J=2.4 Hz, 1H), 7.52-7.27 (m, 2H), 7.18-7.11 (m, 1H), 6.91 (d, J=2.4 Hz, 1H), 4.78-4.57 (m, 3H), 4.51-4.31 (m, 3H), 3.48-3.21 (m, 5H), 3.18-2.85 (m, 5H), 2.12-1.96 (m, 1H), 1.88-1.75 (m, 1H).

### Example 47

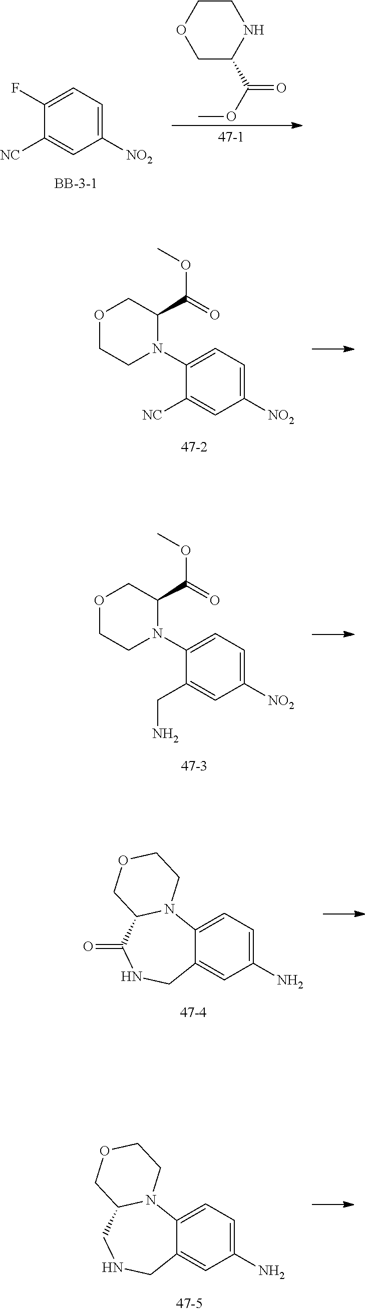

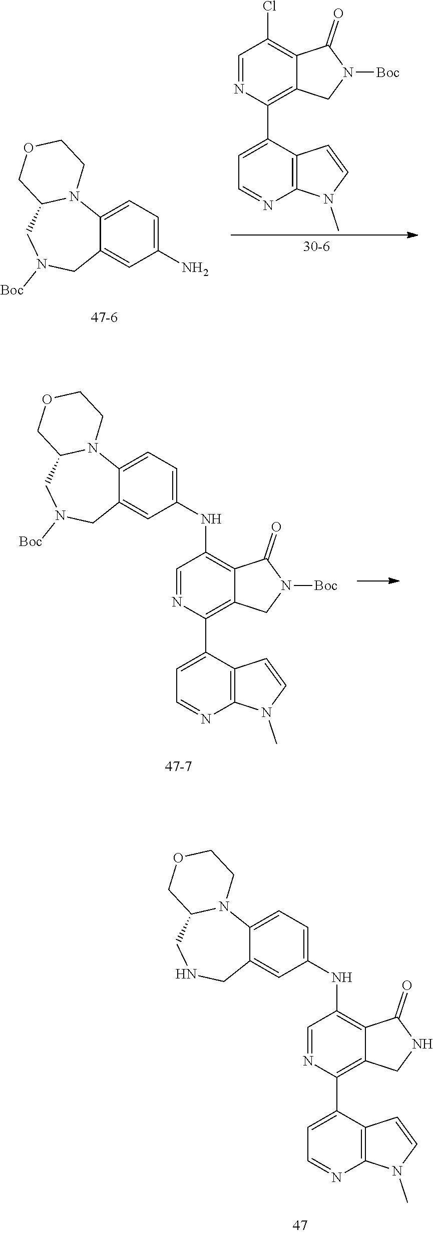 [SMILES: CC(C)(C)OC(=O)N1Cc2cc(N)ccc2N2CCOC[C@H]2C1 ; Cn1ccc2c(-c3ncc(Cl)c4c3CN(C(=O)OC(C)(C)C)C4=O)ccnc21 ; Cn1ccc2c(-c3ncc(Nc4ccc5c(c4)CN(C(=O)OC(C)(C)C)C[C@@H]4COCCN54)c4c3CN(C(=O)OC(C)(C)C)C4=O)ccnc21 ; Cn1ccc2c(-c3ncc(Nc4ccc5c(c4)CNC[C@@H]4COCCN54)c4c3CNC4=O)ccnc21]

### Step 1: Synthesis of Compound 47-2

Compound BB-3-1 (2 g, 13.78 mmol) and compound 47-1 (2.75 g, 16.53 mmol) were added to DMF (10 mL), and DIEA (1.78 g, 13.78 mmol) was added. The mixture was reacted at 100° C. for 16 hours. The reaction solution was added to semi-saturated brine (50 mL), and the mixture was extracted with methyl tert-butyl ether (50 mL×2). The organic phases were combined, dried over anhydrous sodium sulfate, and filtered. The filtrate was concentrated under reduced pressure to give a crude product. The crude product was separated by column chromatography (petroleum ether:ethyl acetate (v/v)=95:5-85:15) to give compound 47-2. MS m/z: 292.1 [M+1]⁺; ¹H NMR (400 MHz, CDCl₃) δ 8.38-8.37 (m, 1H), 8.29-8.26 (m, 1H), 7.13-7.10 (m, 1H), 4.73 (s, 1H), 4.46-4.44 (m, 1H), 3.99-3.93 (m, 2H), 3.77-3.73 (m, 2H), 3.71-3.68 (m, 3H), 3.26-3.22 (m, 1H).

### Step 2: Synthesis of Compound 47-3

The hydrogenation bottle was purged with argon twice, and Raney-Ni (441.23 mg, 5.15 mmol) was added. MeOH (2 mL) and compound 47-2 (1.5 g, 5.15 mmol) were added, and the mixture was reacted at 50° C. under hydrogen (50 psi) for 12 hours. The reaction solution was filtered, and the filtrate was concentrated under reduced pressure to give compound 47-3. MS m/z: 266.0[M+1]⁺.

### Step 3: Synthesis of Compound 47-4

Compound 47-3 (0.7 g, 2.64 mmol) was added to MeOH (14 mL), and sodium tert-butoxide (1.27 g, 13.19 mmol) was added. The mixture was reacted at 25° C. for 16 hours. The mixture was concentrated under reduced pressure to give a crude product. The crude product was separated by column chromatography (petroleum ether:ethyl acetate=100:0-0:100 to ethyl acetate:methanol=97:3) to give compound 47-4. MS m/z: 233.9 [M+1]⁺.

### Step 4: Synthesis of Compound 47-5

Compound 47-4 (0.2 g, 857.39 μmol) was added to THF (20 mL). The mixture was cooled to 0° C., and BH₃·THF (1 M, 8.57 mL) was added. The mixture was reacted at 60° C. under nitrogen for 12 hours. Methanol (10 mL) and 2M dilute hydrochloric acid (20 mL) were added dropwise to the reaction solution at room temperature under nitrogen, and the mixture was stirred at 65° C. for 16 hours. The mixture was concentrated under reduced pressure to give a crude product. The crude product was added to a saturated aqueous solution of sodium carbonate (30 mL), and the pH was adjusted to 8. The mixture was extracted with ethyl acetate (20 mL×3). The aqueous phase was concentrated under reduced pressure, and stirred with a mixture of DCM and MeOH (DCM:MeOH=3:1, 50 mL) for 10 min. Then, the mixture was filtered. The organic phases were combined and concentrated under reduced pressure to give compound 47-5. MS m/z: 220.0 [M+1]⁺.

### Step 5: Synthesis of Compound 47-6

Compound 47-5 (0.2 g, 912.07 μmol) was added to THF (6 mL). TEA (276.87 mg, 2.74 mmol) and Boc₂O (199.06 mg, 912.07 μmol) were added. The mixture was reacted at 25° C. for 2 hours. The reaction solution was added to a saturated aqueous solution of ammonium chloride (50 mL), and the mixture was extracted with ethyl acetate (50 mL×2). The organic phases were combined, dried over anhydrous sodium sulfate, filtered, and concentrated under reduced pressure to give a crude product. The crude product was purified by column chromatography (petroleum ether:ethyl acetate=100:0-80:20) to give compound 47-6. MS m/z: 319.6 [M+1]⁺.

### Step 6: Synthesis of Compound 47-7

Compound 30-6 (50 mg, 125.36 μmol) and compound 47-6 (48.05 mg, 150.44 mol) were added to dioxane (4 mL). Cs₂CO₃ (81.69 mg, 250.73 μmol), BINAP (15.61 mg, 25.07 μmol), and Pd(OAc)₂ (2.81 mg, 12.54 μmol) were added, and the mixture was reacted at 100° C. for 2 hours. The reaction solution was added to water (30 mL), and the mixture was extracted with ethyl acetate (30 mL×2). The organic phases were combined, dried over anhydrous sodium sulfate, and filtered. The filtrate was concentrated under reduced pressure to give compound 47-7. MS m/z: 682.4 [M+1]⁺.

### Step 7: Synthesis of a Trifluoroacetate of Compound 47

Compound 47-7 (50 mg, 73.34 μmol) was added to TFA (2 mL) and DCM (4 mL), and the mixture was reacted at 25° C. for 2 hours. The reaction solution was concentrated under reduced pressure to give a crude product. The crude product was purified by prep-HPLC (column: Phenomenex luna C18 80*30 mm*3 μm; mobile phase: [A: aqueous phase (0.1% TFA); B: acetonitrile]; B %: 5%-45%, 8.00 min) to give a trifluoroacetate of compound 47. MS m/z: 482.1 [M+1]⁺; ¹H NMR (400 MHz, DMSO-d₆) δ 9.10 (s, 1H), 8.98-8.92 (m, 2H), 8.76-8.75 (m, 2H), 8.36 (d, J=4.8 Hz, 1H), 7.57-7.56 (m, 1H), 7.48-7.46 (m, 2H), 7.38-7.36 (m, 1H), 7.16 (d, J=8.8 Hz, 1H), 6.89-6.88 (m, 1H), 4.73 (s, 2H), 4.39-4.29 (m, 3H), 3.87 (s, 3H), 3.32-3.20 (m, 5H), 3.05 (s, 2H).

### Example 48 and Example 49

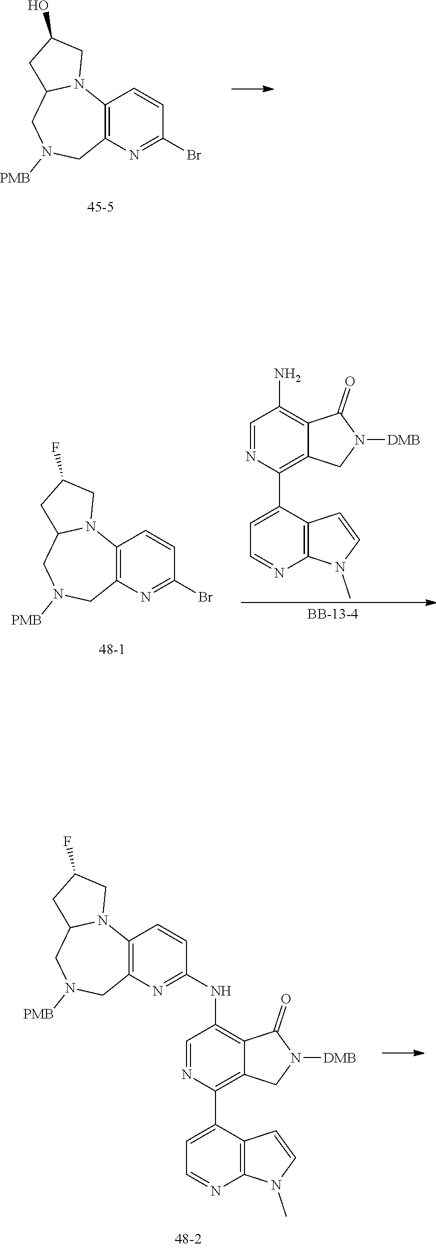

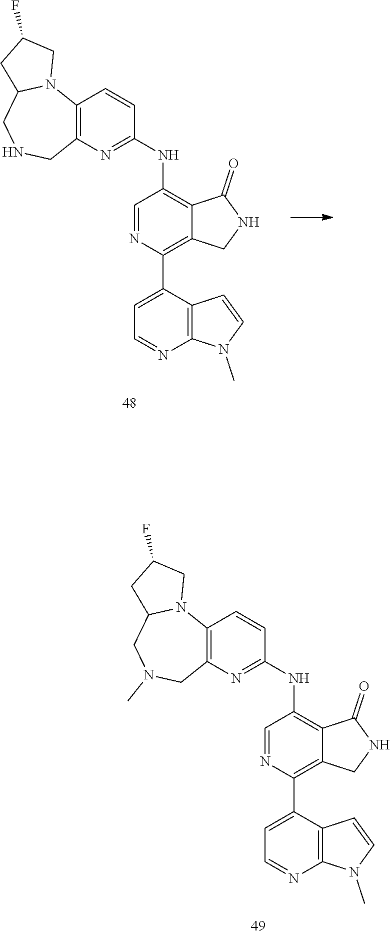 [SMILES: Cn1ccc2c(-c3ncc(Nc4ccc5c(n4)CNCC4C[C@H](F)CN54)c4c3CNC4=O)ccnc21 ; CN1Cc2nc(Nc3cnc(-c4ccnc5c4ccn5C)c4c3C(=O)NC4)ccc2N2C[C@@H](F)CC2C1]

### Step 1: Synthesis of Compound 48-1

Compound 45-5 (255 mg, 630.72 μmol) was dissolved in DCM (5 mL), and the reaction system was purged with nitrogen three times. The mixture was cooled to −78° C., and DAST (508.32 mg, 3.15 mmol) was added dropwise. The mixture was warmed to 25° C. and reacted for 16 hours. A saturated sodium bicarbonate solution (20 mL) was added to quench the reaction. The mixture was extracted with dichloromethane (20 mL×3). The organic phases were combined, washed with saturated brine (20 mL), dried over anhydrous sodium sulfate, and filtered. The filtrate was concentrated under reduced pressure to give a crude product. The crude product was separated by column chromatography (ethyl acetate/petroleum ether=0%-30%) to give compound 48-1. MS m/z: 406.1, 408.0 [M+1]⁺; ¹H NMR (400 MHz, CDCl₃) δ 7.24 (d, J=8.0 Hz, 3H), 6.95-6.84 (m, 3H), 4.31-4.16 (m, 1H), 3.89 (d, J=14.4 Hz, 1H), 3.81 (s, 3H), 3.78-3.59 (m, 3H), 3.58-3.46 (m, 1H), 3.44-3.25 (m, 2H), 2.98 (d, J=11.6 Hz, 1H), 2.76-2.62 (m, 1H), 2.59-2.39 (m, 1H), 1.94-1.78 (m, 1H).

### Step 2: Synthesis of Compound 48-2

Compound BB-13-4 (31 mg, 72.18 μmol) was dissolved in 1,4-dioxane (2 mL) under nitrogen. Compound 48-1 (29.33 mg, 72.18 μmol), cesium carbonate (70.55 mg, 216.55 mol), Pd₂(dba)₃ (6.61 mg, 7.22 μmol), and Ruphos (6.74 mg, 14.44 μmol) were added. The mixture was reacted at 110° C. for 2 hours. The mixture was cooled and filtered. The filter cake was washed with dichloromethane (20 mL). The filtrate was concentrated under reduced pressure to give a crude product. The crude product was separated by column chromatography (ethyl acetate/petroleum ether=0%-100%) to give compound 48-2. MS m/z: 755.3 [M+Na]⁺.

### Step 3: Synthesis of a Trifluoroacetate of Compound 48

Compound 48-2 (49 mg, 64.91 μmol) was dissolved in TfOH (11.30 mmol, 1 mL) under nitrogen, and the mixture was reacted at 40° C. for 5 hours. The mixture was cooled to room temperature, and the system was slowly added to dilute aqueous ammonia (10 mL). The mixture was extracted with dichloromethane:methanol=10:1 (20 mL×3). The organic phases were combined, dried over anhydrous sodium sulfate, filtered and concentrated under reduced pressure. The resulting crude product was purified by prep-HPLC (column: Phenomenex luna C18 80*30 mm*3 μm; mobile phase: [A: aqueous phase (0.1% TFA); B: acetonitrile]; B %: 5%-35%, 8.00 min) to give a trifluoroacetate of compound 48. MS m/z: 485.2 [M+1]⁺; ¹H NMR (400 MHz, DMSO-d₆) δ 10.02 (s, 1H), 9.68 (s, 1H), 9.50-9.36 (m, 1H), 9.23 (s, 1H), 8.36 (d, J=4.8 Hz, 1H), 7.58 (d, J=3.6 Hz, 1H), 7.49-7.33 (m, 2H), 7.17 (d, J=8.8 Hz, 1H), 6.90 (d, J=3.6 Hz, 1H), 5.55-5.29 (m, 1H), 4.72 (s, 3H), 4.30-4.25 (m, 1H), 3.87 (s, 3H), 3.49-3.47 (m, 4H), 3.14-2.98 (m, 1H), 2.78-2.65 (m, 1H), 2.07-1.90 (m, 1H). The trifluoroacetate of compound 48 was added to a sodium bicarbonate solution. The mixture was extracted with ethyl acetate. The organic phase was dried over anhydrous sodium sulfate and concentrated under reduced pressure to give compound 48.

### Step 4: Synthesis of a Trifluoroacetate of Compound 49

Compound 48 (30 mg, 61.92 μmol) was dissolved in methanol (2 mL) under nitrogen. A 37% aqueous solution of formaldehyde (25.12 mg, 309.58 μmol) and acetic acid (11.15 mg, 185.75 μmol) were added, and the mixture was reacted at 20° C. for 1 hour. NaBH(OAc)₃ (26.25 mg, 123.83 μmol) was added, and the mixture was reacted at 20° C. for 15 hours. A saturated sodium carbonate solution (10 mL) was added, and the mixture was extracted with dichloromethane:methanol=10:1 (20 mL×3). The organic phases were combined, dried over anhydrous sodium sulfate, filtered and concentrated to give a crude product. The crude product was separated by prep-HPLC (column: Phenomenex C18 80*30 mm*3 μm; mobile phase: [water (TFA)-acetonitrile]; acetonitrile %: 5%-35%, 8 min) to give a trifluoroacetate of compound 49. MS m/z: 499.2 [M+1]⁺; ¹H NMR (400 MHz, DMSO-d₆) δ 10.11-9.91 (m, 1H), 9.74 (s, 1H), 9.23 (s, 1H), 8.36 (d, J=4.8 Hz, 1H), 7.63-7.30 (m, 3H), 7.29-7.10 (m, 1H), 6.91 (d, J=3.2 Hz, 1H), 5.59-5.30 (m, 1H), 5.08-4.82 (m, 1H), 4.78-4.40 (50-1 m, 4H), 3.87 (s, 6H), 3.54-3.43 (m, 2H), 3.29 (d, J=1.6 Hz, 1H), 3.14 (s, 2H), 2.90 (d, J=6.0 Hz, 1H).

### Example 50A and Example 50

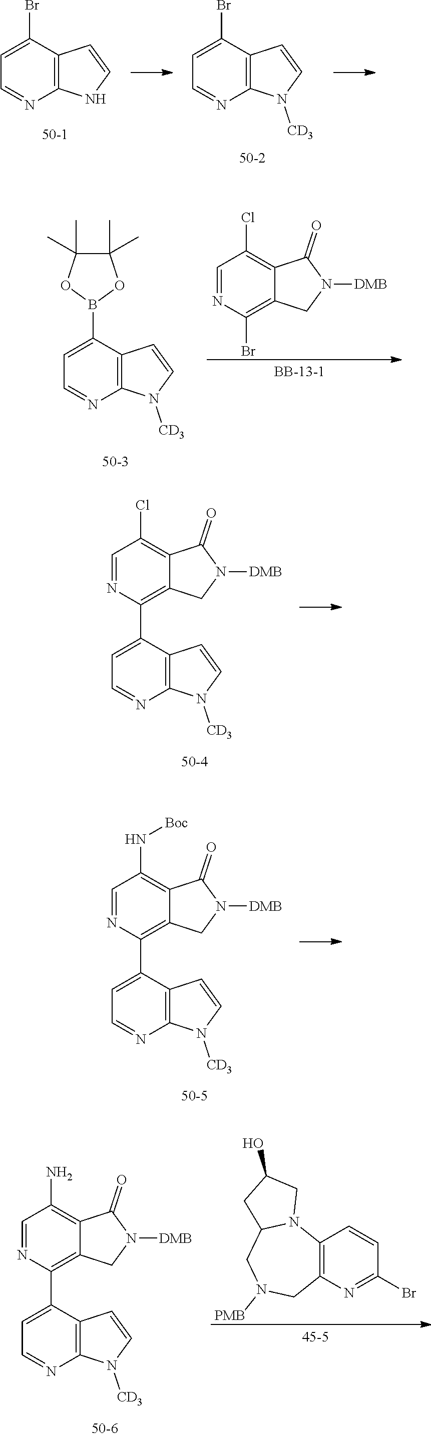

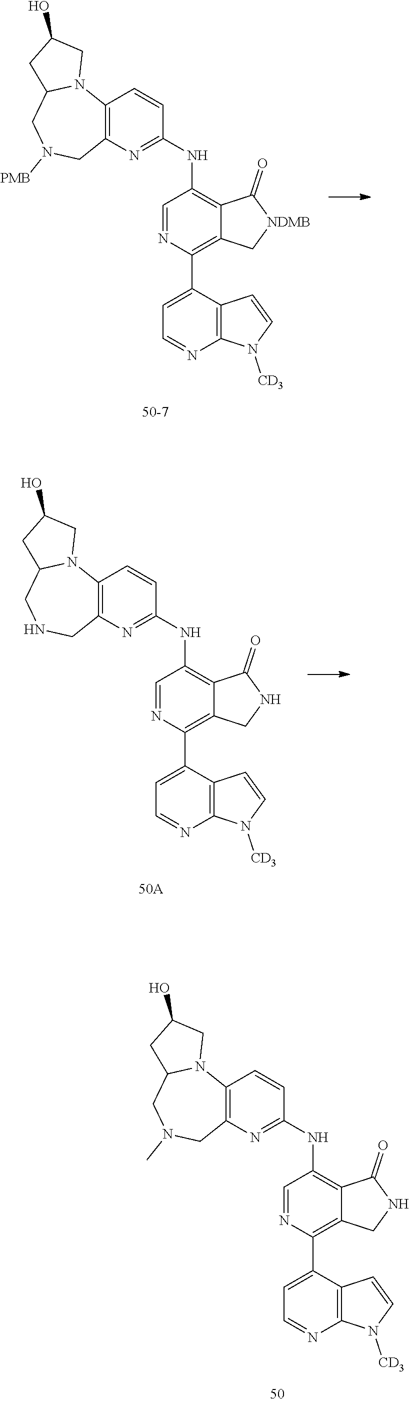

### Step 1: Synthesis of Compound 50-2

Compound 50-1 (1 g, 5.08 mmol) was added to DMF (20 mL), and the mixture was cooled to 0° C. NaH (405.99 mg, 10.15 mmol, 60% content) was added, and the mixture was stirred at 0° C. for 0.5 hours. CD₃I (882.84 mg, 6.09 mmol) was added, and the mixture was reacted at 25° C. for 1.5 hours. After the reaction, the reaction solution was slowly added to water (50 mL). The mixture was extracted with ethyl acetate (50 mL×2). The organic phases were combined, dried over anhydrous sodium sulfate, and filtered. The filtrate was concentrated under reduced pressure. The resulting crude product was separated by column chromatography (PE:EA=100:0-85:15) to give compound 50-2. MS m/z: 214.1, 216.1 [M+1]⁺.

### Step 2: Synthesis of Compound 50-3

Compound 50-2 (0.2 g, 934.24 μmol) and bis(pinacolato)diboron (355.86 mg, 1.40 mmol) were added to dioxane (4 mL). KOAc (275.06 mg, 2.80 mmol) and Pd(dppf)Cl₂·CH₂Cl₂ (76.29 mg, 93.42 μmol) were added. The mixture was reacted at 100° C. for 12 hours. The reaction solution was added to water (30 mL), and the mixture was extracted with ethyl acetate (30 mL×2). The organic phases were combined, dried over anhydrous sodium sulfate, filtered, and concentrated under reduced pressure to give a crude product. The crude product was purified by a preparative chromatography plate (PE:EA=3:1) to give compound 50-3. MS m/z: 180.2 [M-81]⁺.

### Step 3: Synthesis of Compound 50-4

Compound BB-13-1 (0.18 g, 452.66 μmol) and compound 50-3 (118.21 mg, 452.66 μmol) were added to dioxane (4 mL) and water (0.8 mL). Pd(dppf)Cl₂ (33.12 mg, 45.27 mol) and potassium carbonate (125.12 mg, 905.32 μmol) were added. The mixture was reacted at 100° C. for 6 hours. The mixture was cooled to room temperature, and slowly added dropwise to 6 mL of methanol with stirring. After the addition was completed, the mixture was stirred for 10 min. The mixture was filtered, and the filter cake was dried under reduced pressure to give compound 50-4. MS m/z: 452.1[M+H]⁺.

### Step 4: Synthesis of Compound 50-5

Compound 50-4 (0.12 g, 265.53 μmol) and Boc₂O (62.21 mg, 531.07 μmol) were added to dioxane (4 mL). Potassium phosphate (169.09 mg, 796.60 μmol), RuPhos (24.78 mg, 53.11 μmol), and Pd₂(dba)₃ (24.32 mg, 26.55 μmol) were added. The mixture was reacted at 110° C. for 2 hours. The reaction solution was added to 20 ml of water, and the mixture was extracted with ethyl acetate (20 mL×2). The organic phases were combined, dried over anhydrous sodium sulfate, and filtered. The filtrate was concentrated under reduced pressure to give a crude product. The crude product was separated by a preparative chromatography plate (PE:EA=1:1) to give compound 50-5. MS m/z: 533.2[M+1]⁺.

### Step 5: Synthesis of Compound 50-6

Compound 50-5 (0.12 g, 225.31 μmol) was added to TFA (3 mL), and the mixture was reacted at 25° C. for 2 hours. The reaction solution was concentrated under reduced pressure, and then 10 ml of dichloromethane was added to dissolve the resulting product. The resulting mixture was poured into 30 ml of saturated aqueous solution of sodium carbonate. The mixture was extracted with dichloromethane (10 mL×2). The organic phases were combined, dried over anhydrous sodium sulfate, and filtered. The filtrate was concentrated under reduced pressure to give compound 50-6. MS m/z: 433.2[M+1]⁺.

### Step 6: Synthesis of Compound 50-7

Compound 50-6 (100.00 mg, 231.22 μmol) and compound 45-5 (93.48 mg, 231.22 μmol) were added to dioxane (4 mL). Cs₂CO₃ (150.67 mg, 462.44 μmol) and Xantphos Pd G₄ (22.25 mg, 23.12 μmol) were added. The mixture was reacted at 100° C. for 2 hours. The reaction solution was added to water (20 mL), and the mixture was extracted with ethyl acetate (20 mL×2). The organic phases were combined, dried over anhydrous sodium sulfate, and filtered. The filtrate was concentrated under reduced pressure to give a crude product. The crude product was separated by a preparative chromatography plate (DCM:MeOH=20:1) to give compound 50-7. MS m/z: 756.3[M+1]⁺.

### Step 7: Synthesis of Compound 50-8

Compound 50-7 (0.1 g, 132.30 μmol) was added to TfOH (2 mL), and the mixture was reacted at 40° C. for 2 hours. The reaction solution was slowly added to 10 ml of aqueous ammonia, and the pH was greater than 10. The mixture was extracted with dichloromethane:methanol=10:1 (20 mL×2). The organic phases were combined, dried over anhydrous sodium sulfate, and filtered. The filtrate was concentrated under reduced pressure to give compound 50A. MS m/z: 486.3 [M+1]⁺.

### Step 8: Synthesis of Compound 50

Compound 50A (80 mg, 164.76 μmol) and a 37% aqueous solution of formaldehyde (823.80 μmol, 61.33 μL) were added to MeOH (2 mL), and AcOH (29.68 mg, 494.28 μmol) was added. The mixture was reacted at 25° C. for 1 hour. NaBH(OAc)₃ (69.84 mg, 329.52 μmol) was added, and the mixture was reacted at 25° C. for 1 hour. The reaction solution was concentrated under reduced pressure to give a crude product. The crude product was separated by prep-HPLC (column: Phenomenex C18 75*30 mm*3 μm; mobile phase: [water (TFA)-acetonitrile]; acetonitrile %: 1%-30%, 8 min) to give a trifluoroacetate of compound 50. MS m/z:500.2 [M+1]⁺; ¹H NMR (400 MHz, DMSO-d₆) δ 10.12-9.97 (m, 2H), 9.69-9.49 (m, 1H), 8.39-8.36 (m, 1H), 7.61-7.58 (m, 3H), 7.40-7.19 (m, 1H), 6.91-6.88 (m, 1H), 4.97-4.86 (m, 1H), 4.72 (s, 1H), 4.42-4.41 (m, 1H), 4.38-4.37 (m, 2H), 3.66-3.55 (m, 6H), 3.09-3.08 (m, 2H), 3.92 (s, 1H), 2.07-2.06 (m, 1H), 1.85-1.81 (m, 1H).

### Example 51 and Example 52

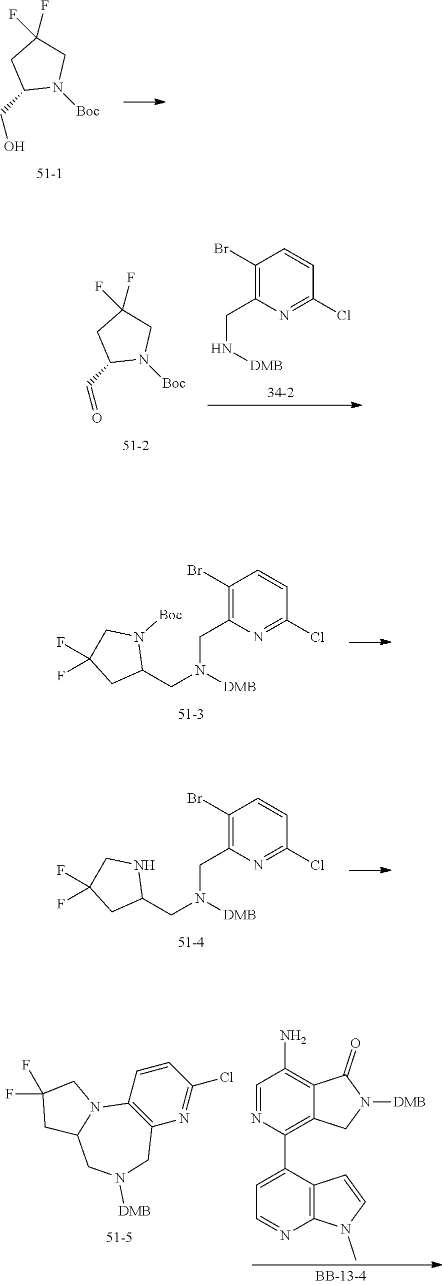

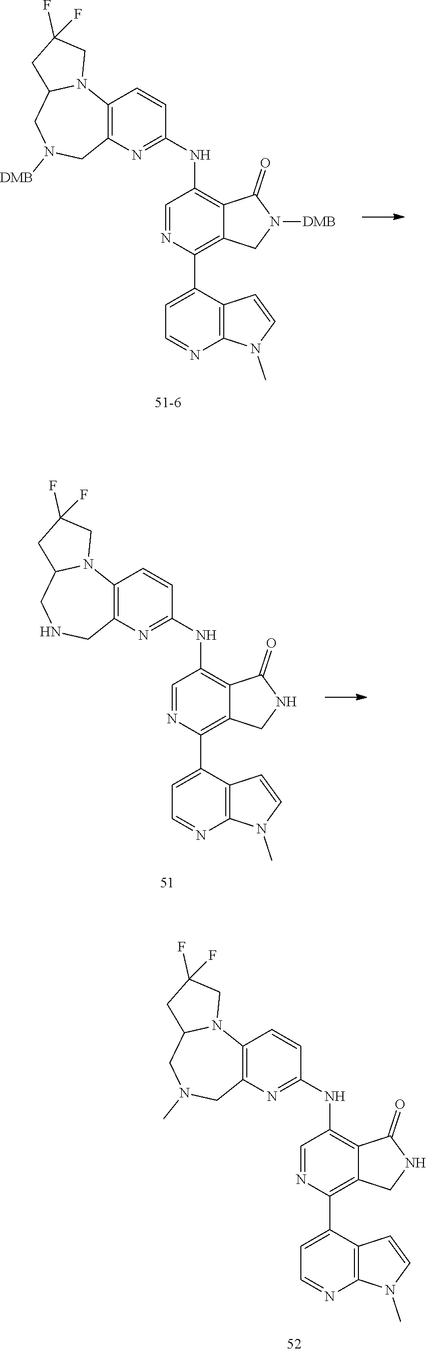

### Step 1: Synthesis of Compound 51-2

Compound 51-1 (0.8 g, 3.37 mmol) was added to DCM (20 mL), and the mixture was cooled to 0° C. Then, Dess-Martin periodinane (1.72 g, 4.05 mmol) was added, and the mixture was reacted at 25° C. for 12 hours. 3 g of sodium carbonate solid was added to the reaction solution, and the mixture was stirred for 0.5 hours. Then, the mixture was filtered. The filtrate was added to a saturated aqueous solution of sodium bicarbonate (20 mL), and the mixture was extracted with dichloromethane (20 mL×2). The organic phases were combined, dried over anhydrous sodium sulfate, and filtered. The filtrate was concentrated under reduced pressure to give a crude product. The crude product was purified by column chromatography (PE:EA=100:0-85:15) to give 51-2.

### Step 2: Synthesis of Compound 51-3

Compound 51-2 (0.2 g, 850.24 μmol) and compound 34-2 (347.60 mg, 935.27 mol) were added to DCM (4 mL), and AcOH (25.53 mg, 425.12 μmol) was added. The mixture was reacted at 25° C. for 1 hour. NaBH(OAc)₃ (360.40 mg, 1.70 mmol) was added, and the mixture was reacted at 25° C. for another 2 hours. The reaction solution was added to a saturated aqueous solution of sodium bicarbonate (20 mL), and the mixture was extracted with dichloromethane (30 mL×2). The organic phases were combined, dried over anhydrous sodium sulfate, and filtered. The filtrate was concentrated under reduced pressure to give compound 51-3. MS m/z: 590.1, 592.1[M+1]⁺.

### Step 3: Synthesis of Compound 51-4

Compound 51-3 (0.38 g, 643.10 μmol) was added to DCM (10 mL), and ZnBr₂ (724.14 mg, 3.22 mmol) was added. The mixture was reacted at 25° C. for 12 hours. The reaction solution was added to 20 mL of saturated aqueous solution of sodium bicarbonate, and the mixture was extracted with dichloromethane (30 mL×3). The organic phases were combined, dried over anhydrous sodium sulfate, and filtered. The filtrate was concentrated under reduced pressure to give compound 51-4. MS m/z: 490.0, 492.0 [M+1]⁺.

### Step 4: Synthesis of Compound 51-5

Compound 51-4 (0.2 g, 407.52 μmol) was added to 1,4-dioxane (9 mL). Cs₂CO₃ (265.56 mg, 815.05 μmol), Pd₂(dba)₃ (37.32 mg, 40.75 μmol), and RuPhos (38.03 mg, 81.50 mol) were added. The mixture was reacted at 105° C. for 2 hours. The reaction solution was added to water (30 mL), and the mixture was extracted with ethyl acetate (30 mL×3). The organic phases were combined, dried over anhydrous sodium sulfate, and filtered. The filtrate was concentrated under reduced pressure to give a crude product. The crude product was separated by column chromatography (PE:EA=100:0-80:20) to give compound 51-5. MS m/z: 410.1 [M+1]⁺.

### Step 5: Synthesis of Compound 51-6

Compound BB-13-4 (60 mg, 139.71 μmol) and compound 51-5 (62.99 mg, 153.68 μmol) were added to 1,4-dioxane (2 mL). Cs₂CO₃ (136.56 mg, 419.12 μmol), Pd₂(dba)₃ (12.79 mg, 13.97 μmol), and RuPhos (13.04 mg, 27.94 μmol) were added. The mixture was reacted at 110° C. for 2 hours. The reaction solution was added to water (20 mL), and the mixture was extracted with ethyl acetate (20 mL×3). The organic phases were combined, dried over anhydrous sodium sulfate, and filtered. The filtrate was concentrated under reduced pressure to give a crude product. The crude product was separated by column chromatography (PE:EA=50:50-20:80) to give compound 51-6. MS m/z: 803.3[M+1]⁺.

### Step 6: Synthesis of a Trifluoroacetate of Compound 51

Compound 51-6 (80 mg, 99.64 μmol) was added to TfOH (2 mL), and the mixture was reacted at 40° C. for 2 hours. The reaction solution was slowly added dropwise to 6 mL of aqueous ammonia, and the mixture was extracted with dichloromethane (10 mL×2). The organic phases were combined, dried over anhydrous sodium sulfate, and filtered. The filtrate was concentrated under reduced pressure to give a crude product. The crude product was separated by prep-HPLC (column: Phenomenex C18 75*30 mm*3 μm; mobile phase: [water (TFA)-acetonitrile]; acetonitrile %: 10%-40%, 8 min) to give a trifluoroacetate of compound 51. MS m/z:503.2[M+1]⁺; ¹H NMR (400 MHz, DMSO-d₆) δ 10.04 (s, 1H), 9.72 (s, 1H), 9.49-9.10 (m, 3H), 8.39-8.36 (m, 1H), 7.61-7.58 (m, 1H), 7.51 (d, J=8.8 Hz, 1H), 7.40-7.39 (m, 1H), 7.21-7.19 (m, 1H), 6.90-6.89 (m, 1H), 4.76-4.73 (m, 2H), 4.65 (s, 1H), 4.62-4.61 (m, 1H), 3.88 (s, 3H), 3.70-3.60 (m, 4H), 3.16-3.10 (m, 2H). The trifluoroacetate of compound 51 was added to a sodium bicarbonate solution, and the mixture was extracted with ethyl acetate. The organic phase was dried over anhydrous sodium sulfate, and concentrated under reduced pressure to give compound 51.

### Step 7: Synthesis of a Trifluoroacetate of Compound 52

Compound 51 (35 mg, 69.65 μmol) and a 37% aqueous solution of formaldehyde (28.26 mg, 348.25 μmol) were added to MeOH (2 mL). AcOH (12.55 mg, 208.95 μmol) was added, and the mixture was stirred at 25° C. for 1 hour. NaBH(OAc)₃ (29.52 mg, 139.30 μmol) was added, and the mixture was reacted at 25° C. for 1 hour. The reaction solution was concentrated under reduced pressure to give a crude product. The crude product was separated by HLPC (column: Phenomenex C18 75*30 mm*3 μm; mobile phase: [water (TFA)-acetonitrile]; acetonitrile %: 10%-40%, 8 min) to give a trifluoroacetate of compound 52. MS m/z:517.2[M+1]⁺; ¹H NMR (400 MHz, DMSO-d₆) δ 10.29-10.10 (m, 1H), 9.74-9.65 (m, 1H), 8.41-8.37 (m, 1H), 7.63-7.60 (m, 2H), 7.41-7.39 (m, 1H), 7.37 (s, 1H), 6.91-6.88 (m, 1H), 4.97 (s, 2H), 4.88 (s, 2H), 4.86-4.72 (m, 1H), 4.68-4.55 (m, 1H), 3.89 (s, 5H), 3.66-3.60 (m, 3H), 3.17-3.14 (m, 2H), 3.10-2.88 (m, 2H).

### Example 53

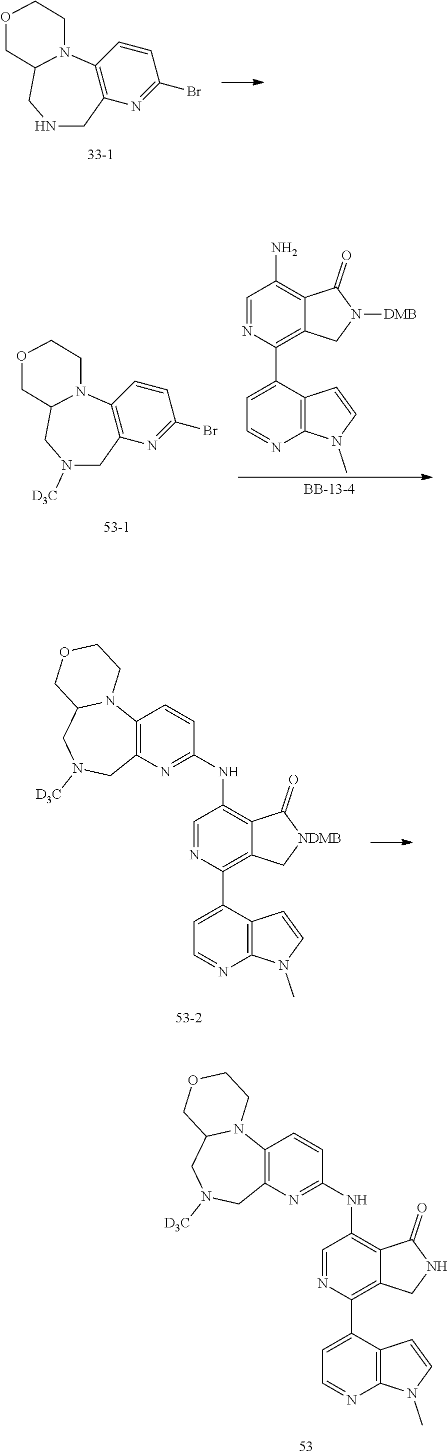

### Step 1: Synthesis of Compound 53-1

Compound 33-1 (55 mg, 193.56 μmol) was added to THF (1 mL). The reaction system was purged with nitrogen three times, and the mixture was cooled to −20° C. NaH (23.22 mg, 580.68 μmol, 60% content) was added, and the mixture was stirred for 0.5 hours. CD₃I (22.45 mg, 154.85 μmol, 9.64 μL) was added, and the mixture was reacted at 20° C. for 2.5 hours. The reaction solution was added to water (20 mL), and the mixture was extracted with ethyl acetate (20 mL). The organic phases were combined, dried over anhydrous sodium sulfate, and filtered. The filtrate was concentrated under reduced pressure to give a crude product. The crude product was separated by a preparative chromatography plate (EA:MeOH=20:1) to give compound 53-1. MS m/z:301.1, 303.1[M+1]⁺.

### Step 2: Synthesis of Compound 53-2

Compound BB-13-4 (20 mg, 46.57 μmol) and compound 53-1 (15.43 mg, 51.23 mol) were added to dioxane (2 mL). Xantphos Pd G₄ (4.48 mg, 4.66 μmol) and Cs₂CO₃ (45.52 mg, 139.71 μmol) were added, and the mixture was reacted at 110° C. for 2 hours. The reaction solution was added to water (20 ml), and the mixture was extracted with ethyl acetate (20 mL×2). The organic phases were combined, dried over anhydrous sodium sulfate, and filtered. The filtrate was concentrated under reduced pressure to give compound 53-2. MS m/z: 650.3 [M+1]⁺.

### Step 3: Synthesis of a Trifluoroacetate of Compound 53

Compound 53-2 (20 mg, 30.78 μmol) was added to TfOH (1 mL), and the mixture was reacted at 40° C. for 1 hour. The reaction solution was slowly added dropwise to aqueous ammonia (4 mL). The mixture was extracted with dichloromethane (10 mL×2). The organic phases were combined, dried over anhydrous sodium sulfate, and filtered. The filtrate was concentrated under reduced pressure to give a crude product. The crude product was separated by prep-HPLC (column: Phenomenex C18 75*30 mm*3 μm; mobile phase: [water (TFA)-acetonitrile]; acetonitrile %: 5%-35%, 8 min) to give a trifluoroacetate of compound 53. MS m/z:500.2[M+1]⁺; ¹H NMR (400 MHz, DMSO-d₆) δ 10.28-10.23 (m, 1H), 10.03-10.02 (m, 1H), 9.77-9.72 (m, 1H), 9.23 (s, 1H), 8.37 (d, J=5.0 Hz, 1H), 7.63-7.59 (m, 2H), 7.40 (d, J=5.0 Hz, 1H), 7.24-7.19 (m, 1H), 6.91 (d, J=3.4 Hz, 1H), 4.78-4.73 (m, 3H), 3.88 (s, 4H), 3.78 (s, 5H), 3.74-3.71 (m, 3H).

### Example 54 and Example 57

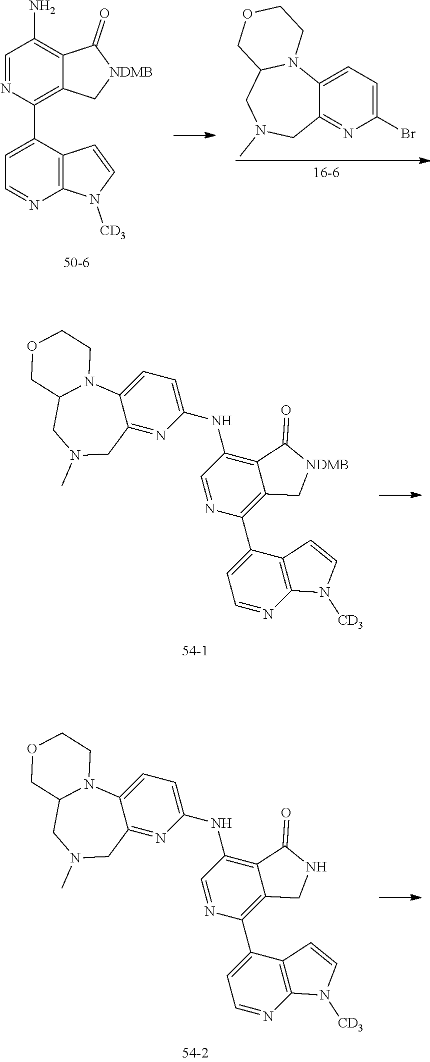

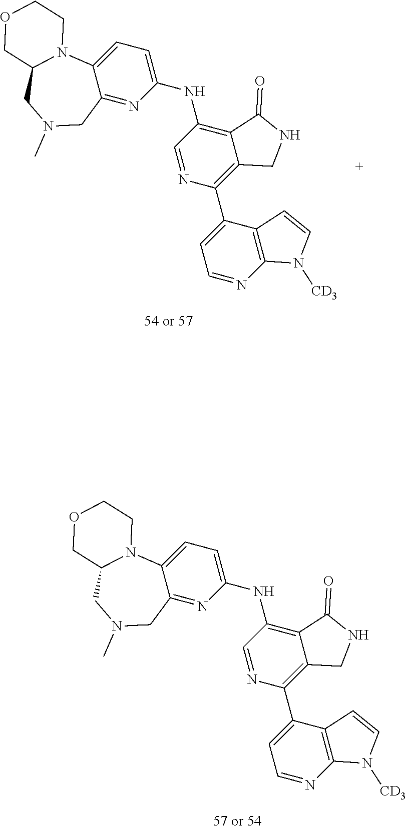 [SMILES: [2H]C([2H])([2H])n1ccc2c(-c3ncc(Nc4ccc5c(n4)CN(C)C[C@H]4COCCN54)c4c3CNC4=O)ccnc21 ; [2H]C([2H])([2H])n1ccc2c(-c3ncc(Nc4ccc5c(n4)CN(C)C[C@@H]4COCCN54)c4c3CNC4=O)ccnc21]

### Step 1: Synthesis of Compound 54-1

Compound 50-6 (50 mg, 115.61 μmol) and compound 16-6 (37.92 mg, 127.17 mol) were added to dioxane (1 mL). Xantphos Pd G₄ (22.25 mg, 23.12 μmol) and Cs₂CO₃ (113.00 mg, 346.83 μmol) were added. The mixture was reacted at 110° C. for 2 hours. The reaction solution was added to water (20 mL), and the mixture was extracted with ethyl acetate (20 mL×2). The organic phases were combined, dried over anhydrous sodium sulfate, and filtered. The filtrate was concentrated under reduced pressure to give compound 54-1. MS m/z:650.3 [M+1]⁺.

### Step 2: Synthesis of a Trifluoroacetate of Compound 54-2

Compound 54-1 (50 mg, 76.95 μmol) was added to TfOH (2 mL), and the mixture was reacted at 40° C. for 12 hours. The reaction solution was added to aqueous ammonia (6 mL), and the mixture was extracted with dichloromethane (10 mL×2). The organic phases were combined, dried over anhydrous sodium sulfate, and filtered. The filtrate was concentrated under reduced pressure to give a crude product. The crude product was separated by prep-HPLC (column: Phenomenex C18 75*30 mm*3 μm; mobile phase: [water (TFA)-acetonitrile]; acetonitrile %: 5%-35%, 8 min) to give a trifluoroacetate of compound 54-2. MS m/z:500.2[M+1]⁺; ¹H NMR (400 MHz, DMSO-d₆) δ 10.34-10.28 (m, 1H), 10.02 (s, 1H), 9.76-9.71 (m, 1H), 9.22 (s, 1H), 8.36 (d, J=5.0 Hz, 1H), 7.62-7.58 (m, 2H), 39-7.38 (m, 1H), 7.22-7.18 (m, 1H), 6.90 (d, J=3.4 Hz, 1H), 4.75-4.72 (m, 4H), 4.63-4.60 (m, 1H), 3.47-3.26 (m, 6H), 3.05 (s, 4H).

### Step 3: Synthesis of Compound 54, Compound 57, a Trifluoroacetate of Compound 54 and a Trifluoroacetate of Compound 57

The trifluoroacetate of compound 54-2 (30.04 g) was subjected to SFC chiral separation (chromatography column: DAICEL CHIRALPAK IC (250 mm*25 mm, 10 m); mobile phase: A n-heptane, B isopropanol/acetonitrile=2/1, 0.1% aqueous ammonia, B %: 40%), and the separated liquid was concentrated to give compound 54 and compound 57.

Compound 54: ¹H NMR (DMSO-d₆, 400 MHz) δ 9.97 (s, 1H), 9.59 (s, 1H), 9.18 (s, 1H), 8.35 (d, 1H, J=4.8 Hz), 7.57 (d, 1H, J=3.6 Hz), 7.44 (d, 1H, J=8.8 Hz), 7.38 (d, 1H, J=5.0 Hz), 7.01 (d, 1H, J=8.8 Hz), 6.91 (d, 1H, J=3.2 Hz), 4.71 (s, 2H), 3.91-4.09 (m, 2H), 3.74-3.77 (m, 3H), 3.67-3.70 (m, 1H), 3.10-3.40 (m, 6H), 2.48-2.55 (m, 2H); ee %=89.99%; retention time: 3.160 min.

Compound 57: ¹H NMR (DMSO-d₆, 400 MHz) δ 9.97 (s, 1H), 9.59 (s, 1H), 9.19 (s, 1H), 8.35 (d, 1H, J=5.0 Hz), 7.58 (d, 1H, J=3.5 Hz), 7.57 (d, 1H, J=8.8 Hz), 7.38 (d, 1H, J=5.0 Hz), 7.06-7.08 (m, 1H), 6.91 (d, 1H, J=3.2 Hz), 4.71 (s, 2H), 3.94-4.16 (m, 2H), 3.67-3.76 (m, 3H), 3.50-3.52 (m, 1H), 3.18-3.31 (m, 6H), 2.48-2.68 (m, 2H); ee %=95.86%; retention time: 3.681 min.

The analysis method of ee % detection: (chromatography column: DAICEL CHIRALPAK IC (50*4.6 mm 3 m); mobile phase: A n-heptane (0.1% DEA), B isopropanol/acetonitrile=2/1, B %: 45%).

Compound 54 (440 mg, 880 μmol) was dissolved in DCM (5 mL), and TFA (4.40 mmol, 327.12 μL) was added. The mixture was stirred at 25° C. for 1 hour. The mixture was directly concentrated under reduced pressure to give a trifluoroacetate of compound 54. MS m/z:250.7[(M+1)/2]⁺; ee %=98.97%; ¹H NMR (DMSO-d₆, 400 MHz) δ 9.97 (s, 1H), 9.72 (s, 1H), 9.23 (s, 1H), 8.37 (d, 1H, J=5.2 Hz), 7.59-7.61 (m, 2H), 7.40 (d, 1H, J=5.2 Hz), 7.20 (br d, 1H, J=8.8 Hz), 6.91 (d, 1H, J=3.6 Hz), 4.71 (s, 2H), 3.71-3.79 (m, 4H), 3.36-3.61 (m, 4H), 3.3-3.5 (m, 3H), 3.01 (s, 3H).

Compound 57 (1.16 g, 2.32 mmol) was dissolved in DCM (12 mL), and TFA (11.61 mmol, 862.39 μL) was added. The mixture was stirred at 25° C. for 1 hour. The mixture was directly concentrated under reduced pressure to give a trifluoroacetate of compound 57. MS m/z:250.8 [(M+1)/2]⁺; ee %=96.40%; ¹H NMR (400 MHz, DMSO-d₆) δ ppm 10.02 (s, 1H), 9.73 (s, 1H), 9.26 (s, 1H), 8.38 (d, J=4.80 Hz, 1H), 7.57-7.61 (m, 2H), 7.40-7.43 (m, 2H), 6.93 (d, J=3.2 Hz, 1H), 4.73 (s, 2H), 3.71-3.79 (m, 5H), 3.26-3.46 (m, 4H), 3.3-3.5 (m, 3H), 3.01 (s, 3H).

### Example 55 and Example 56

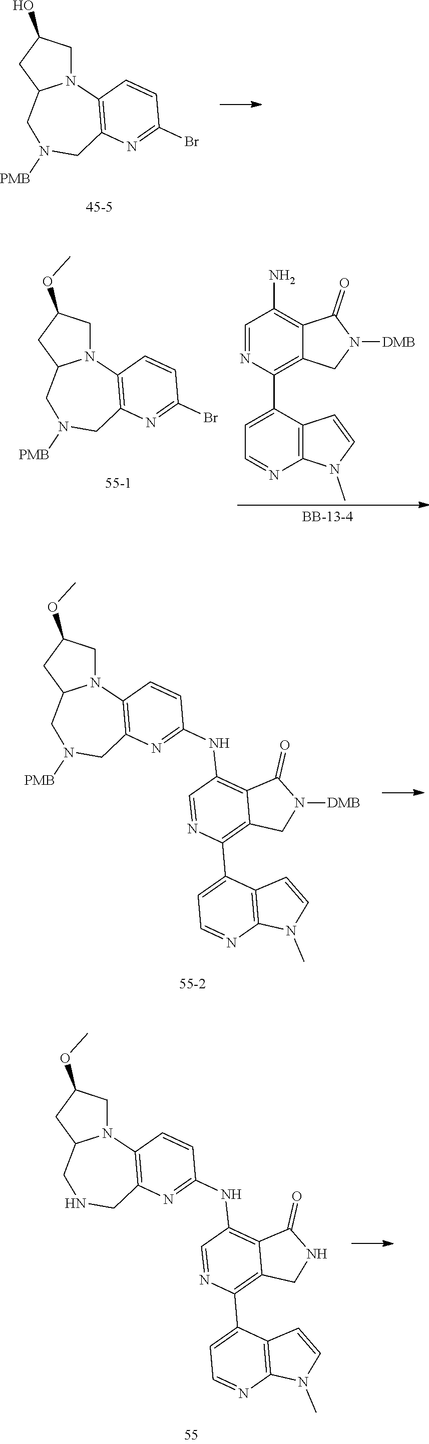

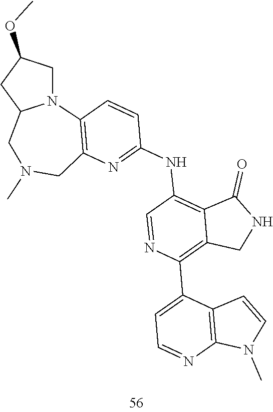 [SMILES: CO[C@@H]1CC2CN(C)Cc3nc(Nc4cnc(-c5ccnc6c5ccn6C)c5c4C(=O)NC5)ccc3N2C1]

### Step 1: Synthesis of Compound 55-1

Compound 45-5 (300 mg, 742.02 μmol) was dissolved in THF (5 mL) in a dry vial. NaH (89.04 mg, 2.23 mmol, 60% purity) was slowly added at 0° C. under nitrogen. The mixture was stirred for 30 minutes, and then Mel (84.26 mg, 593.62 μmol) was slowly added dropwise. The reaction solution was warmed to 15° C. and stirred for another 16 hours. The reaction solution was poured into a saturated ammonium chloride solution (50 mL), and the mixture was extracted with ethyl acetate (20 mL×3). The organic phases were combined, dried over anhydrous sodium sulfate, filtered, and concentrated to give a crude product. The crude product was separated by column chromatography (petroleum ether:ethyl acetate=30-100%) to give compound 55-1. MS m/z: 417.9 [M+H]⁺; ¹H NMR (400 MHz, CDCl₃) δ 7.27-7.22 (m, 3H), 6.91-6.85 (m, 3H), 4.31-4.27 (m, 1H), 4.14-4.06 (m, 1H), 3.81-3.76 (m, 4H), 3.69-3.64 (m, 3H), 3.48-3.45 (m, 1H), 3.38-3.34 (m, 3H), 3.23-3.21 (m, 1H), 2.97-2.94 (m, 1H), 2.55-2.51 (m, 1H), 2.08-2.05 (m, 1H), 1.76-1.73 (m, 1H).

### Step 2: Synthesis of Compound 55-2

Compound 55-1 (179 mg, 427.90 μmol) and compound BB-13-4 (275.65 mg, 641.84 μmol) were dissolved in 1,4-dioxane (5 mL) in a dry vial. Cs₂CO₃ (418.25 mg, 1.28 mmol) was added, and the pH of the system was tested to be greater than 9. Xantphos Pd G₄ (82.36 mg, 85.58 μmol) was added, and the reaction system was purged with nitrogen 3 times. The mixture was stirred at 110° C. for 4 hours and filtered. The filter cake was washed with ethyl acetate (10 mL×2), and the filtrate was concentrated. The resulting crude product was separated by column chromatography (dichloromethane:methanol=100:0-10:1) to give compound 55-2. MS m/z: 767.5 [M+1]⁺.

### Step 3: Synthesis of a Trifluoroacetate of Compound 55

Compound 55-2 (50.18 mg, 65.43 μmol) was added to TfOH (1 mL), and the mixture was reacted at 40° C. for 2 hours. The reaction solution was cooled, and then slowly added dropwise to aqueous ammonia (4 mL). The mixture was extracted with dichloromethane (20 mL×2). The organic phases were combined, dried over anhydrous sodium sulfate, and filtered. The filtrate was concentrated under reduced pressure to give a crude product. The crude product was separated by prep-HPLC (column: Phenomenex C18 75*30 mm*3 μm; mobile phase: [water (TFA)-acetonitrile]; acetonitrile %: 5%-35%, 8 min) to give a trifluoroacetate of compound 55. MS m/z: 497.2 [M+1]⁺; ¹H NMR (400 MHz, DMSO-d₆) δ 10.00 (s, 1H), 9.64 (s, 1H), 9.30-9.29 (m, 1H), 9.20 (s, 1H), 9.04-9.01 (m, 1H), 8.36 (d, J=5.0 Hz, 1H), 7.57 (d, J=3.4 Hz, 1H), 7.47 (d, J=9.0 Hz, 1H), 7.38 (d, J=5.0 Hz, 1H), 7.15 (d, J=8.8 Hz, 1H), 6.89 (d, J=3.6 Hz, 1H), 4.72 (s, 2H), 4.68-4.63 (m, 1H), 4.23-4.18 (m, 1H), 4.11-4.08 (m, 1H), 3.87 (s, 4H), 3.33 (s, 4H), 3.10-3.08 (m, 2H), 2.18-2.14 (m, 1H), 1.97-1.93 (m, 1H).

### Step 4: Synthesis of a Trifluoroacetate of Compound 56

The trifluoroacetate of compound 55 (50 mg, 100.69 μmol) and a 37% aqueous solution of formaldehyde (40.86 mg, 503.46 μmol) were added to MeOH (2 mL). AcOH (18.14 mg, 302.08 μmol) was added, and the mixture was reacted at 25° C. for 1 hour. NaBH(OAc)₃ (42.68 mg, 201.38 μmol) was added, and the mixture was reacted for 2 hours. The reaction solution was concentrated under reduced pressure to give a crude product. The crude product was separated by prep-HPLC (column: Phenomenex C18 75*30 mm*3 μm; mobile phase: [water (TFA)-acetonitrile]; acetonitrile %: 5%-35%, 8 min) to give a trifluoroacetate of compound 56. MS m/z: 511.3 [M+H]⁺; ¹H NMR (400 MHz, DMSO-d₆) δ 10.15-9.96 (m, 2H), 9.70-9.60 (m, 1H), 9.21-9.19 (m, 1H), 8.37-8.36 (m, 1H), 7.59-7.51 (m, 2H), 7.40-7.38 (m, 1H), 7.19-7.14 (m, 1H), 6.91-6.90 (m, 1H), 4.76-4.72 (m, 3H), 4.46-4.43 (m, 1H), 4.22-4.00 (m, 2H), 3.88 (s, 3H), 3.67-3.60 (m, 3H), 3.31-3.26 (m, 3H), 3.09-3.08 (m, 2H), 2.91 (br s, 1H), 2.21-2.19 (m, 1H), 1.90-1.88 (m, 1H).

### Example 58

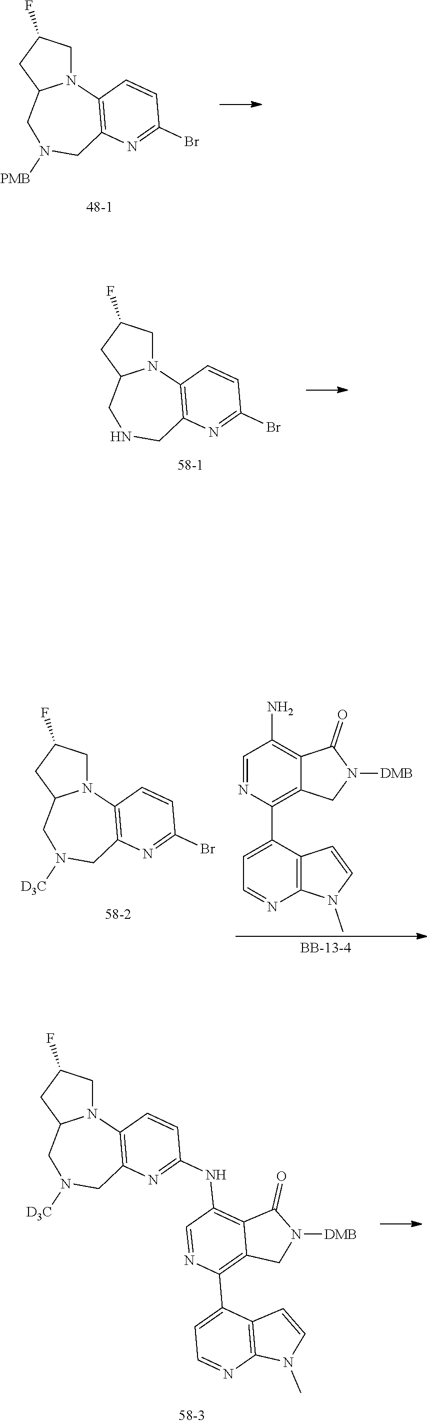

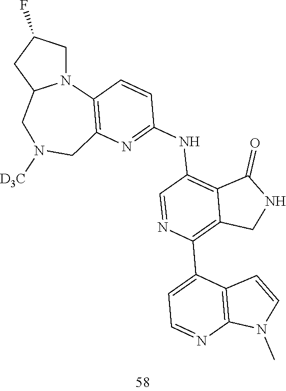 [SMILES: [2H]C([2H])([2H])N1Cc2nc(Nc3cnc(-c4ccnc5c4ccn5C)c4c3C(=O)NC4)ccc2N2C[C@@H](F)CC2C1]

### Step 1: Synthesis of Compound 58-1

Compound 48-1 (140 mg, 213.84 μmol) was dissolved in DCM (4 mL) under nitrogen. TfOH (2.77 g, 18.46 mmol) was added, and the mixture was reacted at 20° C. for 2 hours. Aqueous ammonia (3 mL) was slowly added to the system, and the layers were separated. The organic phase was dried over anhydrous sodium sulfate, and filtered. The filtrate was concentrated under reduced pressure to give compound 58-1. MS m/z: 286.0, 288.0 [M+1]⁺.

### Step 2: Synthesis of Compound 58-2

Compound 58-1 (67 mg, 234.15 μmol) was dissolved in tetrahydrofuran (1 mL) under nitrogen. Sodium hydride (28.10 mg, 702.45 μmol, 60% purity) was added at −20° C., and the mixture was reacted at −20° C. for 0.5 hours. A solution of CD₃I (27.15 mg, 187.32 μmol) in tetrahydrofuran (1 mL) was slowly added dropwise, and the mixture was reacted at 20° C. for 15.5 hours. The mixture was cooled to 0° C., and saturated ammonium chloride solution (5 mL) was added. The mixture was extracted with ethyl acetate (20 mL×3). The organic phases were combined, dried over anhydrous sodium sulfate, filtered and concentrated to give a crude product. The crude product was separated by column chromatography (methanol/dichloromethane=0%-5%) to give compound 58-2. MS m/z: 303.1, 305.1 [M+1]⁺.

### Step 3: Synthesis of Compound 58-3

Compound BB-13-4 (38 mg, 88.48 μmol) was dissolved in 1,4-dioxane (1 mL) under nitrogen. Compound 58-2 (26.83 mg, 88.48 μmol), cesium carbonate (86.49 mg, 265.44 mol), Ruphos (8.26 mg, 17.70 μmol), and Pd₂(dba)₃ (8.10 mg, 8.85 μmol) were added. The mixture was reacted at 110° C. for 2 hours. The mixture was cooled to room temperature, and filtered. The filter cake was washed with dichloromethane (20 mL). The filtrate was concentrated under reduced pressure to give a crude product. The crude product was separated by column chromatography (methanol/dichloromethane=0%-15%) to give compound 58-3. MS m/z: 652.3 [M+1]⁺.

### Step 4: Synthesis of a Trifluoroacetate of Compound 58

Compound 58-3 (54.00 mg, 82.85 μmol) was dissolved in DCM (3 mL) under nitrogen. TfOH (1.70 g, 11.30 mmol) was added, and the mixture was reacted at 40° C. for 16 hours. The mixture was cooled to room temperature, and the system was slowly added to aqueous ammonia (10 mL). The mixture was extracted with dichloromethane:methanol=10:1 (20 mL×4). The organic phases were combined, dried over anhydrous sodium sulfate, filtered and concentrated under reduced pressure to give a crude product. The crude product was separated by prep-HPLC (column: Phenomenex C18 80*30 mm*3 μm; mobile phase: [water (TFA)-acetonitrile]; acetonitrile %: 1%-30%, 8 min) to give a trifluoroacetate of compound 58. MS m/z: 502.1 [M+1]⁺; ¹H NMR (400 MHz, DMSO-d₆) δ 9.88-9.60 (m, 1H), 9.39-9.09 (m, 1H), 8.36-8.35 (m, 1H), 7.68-7.28 (m, 4H), 7.24-7.05 (m, 1H), 7.00-6.80 (m, 1H), 5.60-5.26 (m, 1H), 5.20-4.86 (m, 1H), 4.81-4.58 (m, 3H), 4.57-4.28 (m, 2H), 3.96-3.77 (m, 6H), 2.23-1.66 (m, 2H).

### Example 61

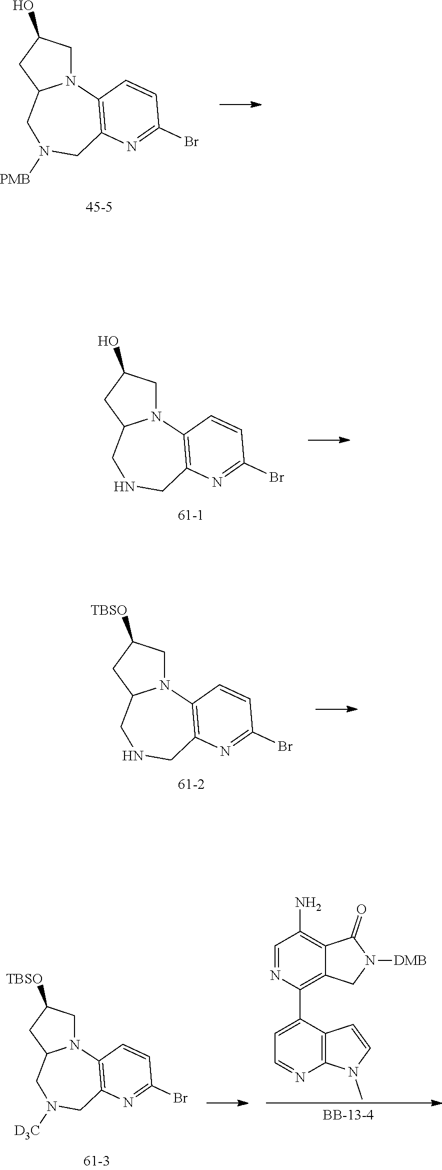

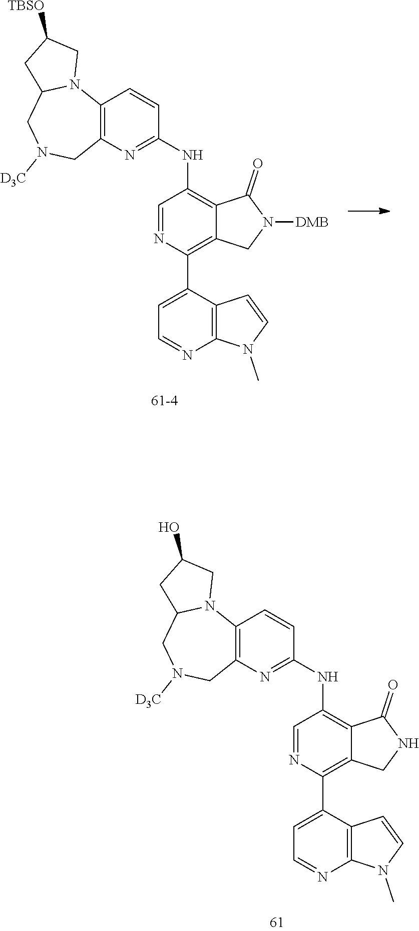

### Step 1: Synthesis of Compound 61-1

Compound 45-5 (500 mg, 1.24 mmol) was dissolved in DCM (10 mL) in a dry vial. TfOH (9.28 g, 61.84 mmol) was added, and the reaction solution was stirred at 40° C. for 16 hours. The reaction solution was poured into 5% dilute aqueous ammonia (100 mL). The aqueous phase was extracted with dichloromethane (30 mL×3). The organic phases were combined, dried over anhydrous sodium sulfate, filtered and concentrated to give a crude product. The crude product was separated by column chromatography (petroleum ether:ethyl acetate=1:1-dichloromethane:methanol=10:1) to give compound 61-1. MS m/z: 284.1, 286.1 [M+1]⁺.

### Step 2: Synthesis of Compound 61-2

Compound 61-1 (135 mg, 475.10 μmol) was dissolved in THF (5 mL) in a dry vial. Imidazole (97.03 mg, 1.43 mmol) and TBSCl (214.82 mg, 1.43 mmol) were added. The mixture was stirred at 15° C. for 16 hours. The reaction solution was poured into water (50 mL), and the mixture was extracted with ethyl acetate (20 mL×3). The organic phases were combined, dried over anhydrous sodium sulfate, filtered and concentrated to give a crude product. The crude product was separated by column chromatography (petroleum ether:ethyl acetate=5:1-0:100) to give 61-2. MS m/z: 397.8, 397.9 [M+1]⁺.

### Step 3: Synthesis of Compound 61-3

Compound 61-2 (100 mg, 251.00 μmol) was dissolved in THF (0.5 mL) in a dry vial. NaH (30.12 mg, 752.99 μmol, 60% purity) was slowly added at −20° C. under nitrogen. The mixture was stirred for 15 minutes. Then, a solution of CD₃I (28.50 mg, 200.80 μmol) in THF (0.5 mL) was added dropwise. The reaction solution was slowly warmed to 15° C. and stirred for another 16 hours. The reaction solution was poured into a saturated ammonium chloride solution (10 mL). The aqueous phase was extracted with ethyl acetate (5 mL×2). The organic phases were combined, dried over anhydrous sodium sulfate, filtered and concentrated to give a crude product. The crude product was purified by a preparative plate (dichloromethane:methanol 20:1) to give compound 61-3. MS m/z: 415.0, 416.9 [M+1]⁺.

### Step 4: Synthesis of Compound 61-4

Compound 61-3 (15 mg, 36.10 μmol) and compound BB-13-4 (18.61 mg, 43.33 mol) were dissolved in dioxane (1 mL) in a dry vial, and Cs₂CO₃ (35.29 mg, 108.31 μmol) was added. The pH of the system was tested to be greater than 9. Then, Xantphos Pd G₄ (6.95 mg, 7.22 μmol) was added, and the reaction system was purged with nitrogen 3 times. The mixture was reacted at 110° C. under nitrogen for 4 hours. The reaction solution was directly filtered, and the filter cake was washed with ethyl acetate (10 mL×2). The filtrate was concentrated to give a crude product. The crude product was purified by a preparative plate (dichloromethane:methanol=10:1) to give compound 61-4. MS m/z: 764.4 [M+1]⁺.

### Step 5: Synthesis of a Trifluoroacetate of Compound 61

Compound 61-4 (25 mg, 32.72 μmol) was dissolved in TfOH (0.5 mL) in a dry vial, and the reaction solution was stirred at 40° C. for 1 hour. The reaction solution was poured into 5% dilute aqueous ammonia (10 mL), and the aqueous phase was extracted with dichloromethane:methanol=5:1 (5 mL×3). The organic phases were combined, dried over anhydrous sodium sulfate, filtered and concentrated to give a crude product. The crude product was separated by prep-HPLC (column: Phenomenex C18 75*30 mm*3 μm; mobile phase: [water (TFA)-acetonitrile]; acetonitrile %: 5%-35%, 8 min) to give a trifluoroacetate of compound 61. MS m/z: 500.1[M+1]⁺; ¹H NMR (400 MHz, DMSO-d₆) δ 10.49-9.94 (m, 1H), 9.70-9.59 (m, 1H), 9.21-9.19 (m, 1H), 8.36-8.35 (m, 1H), 7.58-7.49 (m, 1H), 7.39-7.38 (m, 2H), 7.18-7.14 (m, 1H), 6.91-6.90 (m, 1H), 4.75-4.45 (m, 3H), 4.39-4.28 (m, 3H), 3.87 (s, 4H), 3.43-3.35 (m, 1H), 3.17 (s, 3H), 2.14-2.00 (m, 1H), 1.83-1.77 (m, 1H).

### Example 62

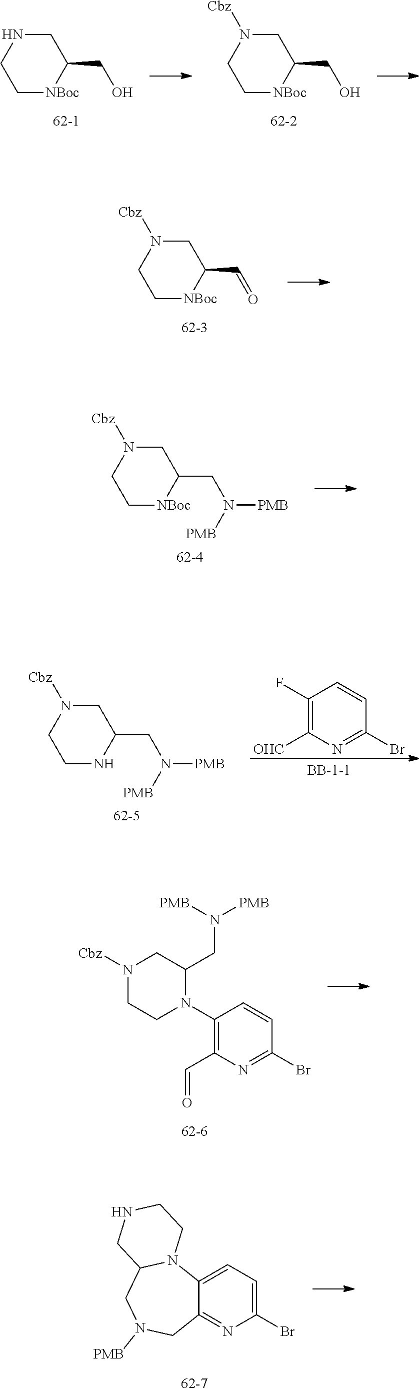

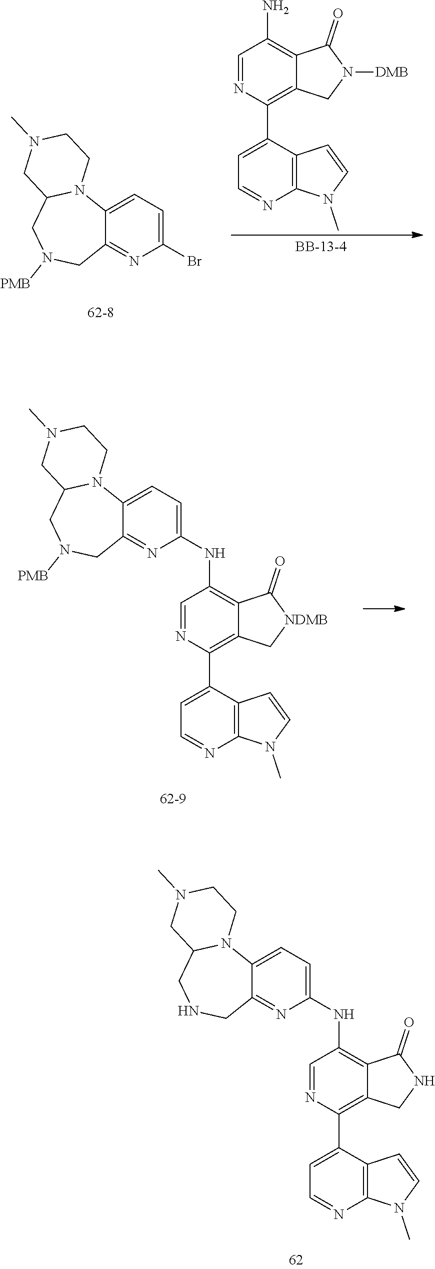

### Step 1: Synthesis of Compound 62-2

Compound 62-1 (1 g, 4.62 mmol) was dissolved in acetonitrile (7 mL) under nitrogen. An aqueous solution (7 mL) of sodium bicarbonate (15.12 g, 14.40 mmol) and benzyl chloroformate (1.18 g, 6.94 mmol) were added. The mixture was reacted at 25° C. for 2 hours. The solvent was concentrated under reduced pressure, and a saturated aqueous solution of sodium carbonate (10 mL) was added. The mixture was extracted with ethyl acetate (20 mL×3). The organic phases were combined, washed with saturated brine (10 mL), dried over anhydrous sodium sulfate, filtered and concentrated under reduced pressure to give a crude product. The crude product was separated by column chromatography (ethyl acetate/petroleum ether=0%-50%) to give compound 62-2. MS m/z: 373.1 [M+Na]⁺; ¹H NMR (400 MHz, CDCl₃) δ 7.48-7.27 (m, 5H), 5.17 (s, 2H), 4.31-4.13 (m, 2H), 4.08-3.94 (m, 1H), 3.93-3.79 (m, 1H), 3.60 (s, 2H), 3.01 (s, 3H), 1.47 (s, 9H).

### Step 2: Synthesis of Compound 62-3

Compound 62-2 (1.41 g, 4.02 mmol) was dissolved in DCM (28 mL) under nitrogen, and Dess-Martin periodinane (1.88 g, 4.43 mmol) was added. The mixture was reacted at 25° C. for 16 hours. A saturated sodium carbonate solution (20 mL) was added, and the mixture was extracted with dichloromethane (20 mL×3). The organic phases were combined, washed with saturated brine (10 mL), dried over anhydrous sodium sulfate, and filtered. The filtrate was concentrated under reduced pressure to give a crude product. The crude product was separated by column chromatography (ethyl acetate/petroleum ether=0%-50%) to give compound 62-3. MS m/z: 371.1 [M+Na]⁺.

### Step 3: Synthesis of Compound 62-4

Compound 62-3 (1.33 g, 3.82 mmol) was added to DCM (25 mL) under nitrogen. (PMB)₂NH (982.35 mg, 3.82 mmol) and acetic acid (229.25 mg, 3.82 mmol) were added. The mixture was reacted at 25° C. for 1 hour. Sodium acetate borohydride (1.21 g, 5.73 mmol) was added, and the mixture was reacted at 25° C. for 1 hour. A saturated aqueous solution of sodium carbonate (20 mL) was added, and the mixture was extracted with dichloromethane (20 mL×3). The organic phases were combined, washed with saturated brine (10 mL), dried over anhydrous sodium sulfate, and filtered. The filtrate was concentrated under reduced pressure to give a crude product. The crude product was separated by column chromatography (ethyl acetate/petroleum ether=0%-40%) to give compound 62-4. MS m/z: 590.4 [M+1]⁺.

### Step 4: Synthesis of Compound 62-5

Compound 62-4 (1.85 g, 3.14 mmol) was dissolved in hydrogen chloride/methanol (4 M, 100 mL) under nitrogen, and the mixture was reacted at 25° C. for 2 hours. The solvent was concentrated under reduced pressure. Dichloromethane (20 mL) and a saturated aqueous solution of sodium carbonate (20 mL) were added, and the mixture was extracted with dichloromethane (20 mL×3). The organic phases were combined, dried over anhydrous sodium sulfate, and filtered. The filtrate was concentrated under reduced pressure to give compound 62-5. MS m/z: 490.2 [M+1]⁺.

### Step 5: Synthesis of Compound 62-6

Compound 62-5 (1.47 g, 3.00 mmol) was dissolved in DMF (15 mL) under nitrogen. Compound BB-1-1 (734.98 mg, 3.60 mmol) and DIEA (1.16 g, 9.01 mmol) were added. The mixture was reacted at 100° C. for 16 hours. The mixture was cooled. Water (20 mL) and a saturated sodium carbonate solution (20 mL) were added, and the mixture was extracted with ethyl acetate (20 mL×3). The organic phases were combined, washed with saturated brine (20 mL), dried over anhydrous sodium sulfate, and filtered. The filtrate was concentrated under reduced pressure to give a crude product. The crude product was separated by column chromatography (ethyl acetate/petroleum ether=0%-30%) to give compound 62-6. MS m/z: 673.0, 675.0 [M+1]⁺.

### Step 6: Synthesis of Compound 62-7

Compound 62-6 (480 mg, 712.59 μmol) was dissolved in trifluoroacetic acid (15.40 g, 135.06 mmol) under nitrogen, and the mixture was reacted at 60° C. for 16 hours. The mixture was cooled, and concentrated under reduced pressure. DCM (10 mL) and sodium acetate borohydride (453.08 mg, 2.14 mmol) were added. The mixture was reacted at 25° C. for 1 hour. A saturated sodium carbonate solution (30 mL) was added. The mixture was extracted with dichloromethane (20 mL×5), washed with saturated brine (20 mL), dried over anhydrous sodium sulfate, and filtered. The filtrate was concentrated under reduced pressure to give a crude product. The crude product was separated by column chromatography (methanol/dichloromethane=0%-20%) to give compound 62-7. MS m/z: 403.0, 405.0 [M+1]⁺; ¹H NMR (400 MHz, CDCl₃) δ 7.30 (d, J=8.4 Hz, 1H), 7.25 (d, J=8.8 Hz, 2H), 7.06 (d, J=8.4 Hz, 1H), 6.86 (d, J=8.4 Hz, 2H), 4.16 (d, J=14.4 Hz, 1H), 3.96 (d, J=14.4 Hz, 1H), 3.80 (s, 3H), 3.75-3.69 (m, 1H), 3.65-3.58 (m, 1H), 3.49 (s, 1H), 3.21-3.13 (m, 2H), 3.10-3.02 (m, 2H), 3.00-2.90 (m, 2H), 2.86-2.81 (m, 1H), 2.63-2.89 (m, 1H).

### Step 7: Synthesis of Compound 62-8

Compound 62-7 (150 mg, 371.92 μmol) was dissolved in methanol (3 mL) under nitrogen. A 37% aqueous solution of formaldehyde (150.91 mg, 1.86 mmol) and acetic acid (67.00 mg, 1.12 mmol) were added, and the mixture was reacted at 25° C. for 1 hour. Sodium acetate borohydride (157.65 mg, 743.84 μmol) was added, and the mixture was reacted at 25° C. for 1 hour. A saturated aqueous solution of sodium carbonate (10 mL) was added, and the mixture was extracted with dichloromethane (10 mL×3). The organic phases were combined, washed with saturated brine (10 mL), dried over anhydrous sodium sulfate, and filtered. The filtrate was concentrated under reduced pressure to give a crude product. The crude product was separated by column chromatography (methanol/ethyl acetate=0%-10%) to give compound 62-8. MS m/z: 417.0, 419.0 [M+1]⁺; ¹H NMR (400 MHz, CDCl₃) δ 7.30 (s, 1H), 7.27-7.22 (m, 2H), 7.06 (d, J=8.4 Hz, 1H), 6.86 (d, J=8.4 Hz, 2H), 4.18-4.07 (m, 1H), 4.03-3.93 (m, 1H), 3.81 (s, 3H), 3.75-3.57 (m, 2H), 3.46-3.32 (m, 2H), 2.90-2.77 (m, 1H), 2.70-2.57 (m, 2H), 2.48-2.33 (m, 3H), 1.87-1.66 (m, 4H).

### Step 8: Synthesis of Compound 62-9

Compound BB-13-4 (70 mg, 162.99 μmol) was dissolved in 1,4-dioxane (2 mL) under nitrogen. Compound 62-8 (81.63 mg, 195.59 μmol), cesium carbonate (159.32 mg, 488.97 mol), and Xantphos Pd G₄ (15.69 mg, 16.30 μmol) were added. The mixture was reacted at 110° C. for 2 hours. The mixture was cooled and vacuum filtered. The residue was washed with dichloromethane (20 mL). The filtrate was concentrated under reduced pressure to give a crude product. The crude product was separated by column chromatography (methanol/dichloromethane=0%-20%) to give compound 62-9. MS m/z: 766.2 [M+1]⁺.

### Step 9: Synthesis of a Trifluoroacetate of Compound 62

Compound 62-9 (50 mg, 65.28 μmol) was dissolved in TfOH (4.25 g, 28.32 mmol) under nitrogen, and the mixture was reacted at 40° C. for 16 hours. The mixture was cooled to room temperature, and the system was slowly added to dilute aqueous ammonia (5%, 10 mL). The mixture was extracted with dichloromethane (20 mL×3). The organic phases were combined, dried over anhydrous sodium sulfate, and filtered. The filtrate was concentrated under reduced pressure to give a crude product. The crude product was separated by prep-HPLC (column: Phenomenex C18 75*30 mm*3 μm; mobile phase: [water (TFA)-acetonitrile]; acetonitrile %: 1%-25%, 8 min) to give a trifluoroacetate of compound 62. MS m/z: 496.3 [M+1]⁺; ¹H NMR (400 MHz, DMSO-d₆) δ 10.04 (s, 1H), 9.75 (s, 1H), 9.23 (s, 1H), 8.36 (d, J=5.2 Hz, 1H), 7.73-7.48 (m, 2H), 7.39 (d, J=4.8 Hz, 1H), 7.29 (d, J=8.8 Hz, 1H), 6.89 (d, J=3.6 Hz, 1H), 4.73 (s, 2H), 4.57-4.48 (m, 1H), 4.45-4.35 (m, 1H), 3.87 (s, 3H), 3.55 (d, J=3.8 Hz, 6H), 3.26-3.16 (m, 4H), 2.91 (s, 3H).

### Example 63

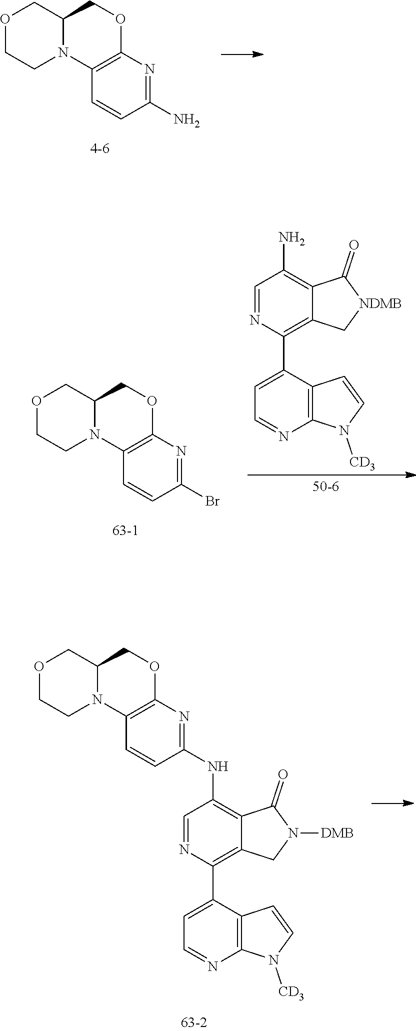

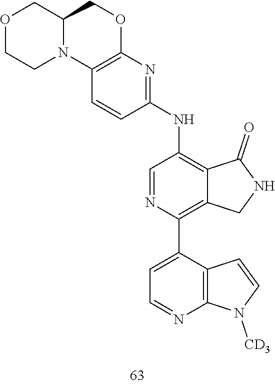 [SMILES: [2H]C([2H])([2H])n1ccc2c(-c3ncc(Nc4ccc5c(n4)OC[C@@H]4COCCN54)c4c3CNC4=O)ccnc21]

### Step 1: Synthesis of Compound 63-1

Compound 4-6 (1 g, 4.83 mmol) was added to 1,2-dibromoethane (10 mL) under nitrogen, and copper bromide (538.90 mg, 2.41 mmol) was added. Isoamyl nitrite (621.85 mg, 5.31 mmol) was slowly added, and the mixture was reacted at 20° C. for 2 hours. Copper bromide (1.08 g, 4.83 mmol) was further added, and the mixture was reacted at 20° C. for 2 hours. The mixture was vacuum filtered, and the filter cake was washed with dichloromethane (20 mL). The filtrate was collected and concentrated under reduced pressure to give a crude product. The crude product was separated by column chromatography (ethyl acetate/petroleum ether=0%-40%) to give compound 63-1. MS m/z: 271.1, 273.1 [M+1]⁺.

### Step 2: Synthesis of Compound 63-2

Compound 50-6 (160 mg, 369.95 μmol) was dissolved in 1,4-dioxane (4 mL) under nitrogen. Compound 63-1 (100.30 mg, 369.95 μmol), potassium phosphate (157.06 mg, 739.90 μmol), Ruphos (51.79 mg, 110.99 μmol), and Pd₂(dba)₃ (67.75 mg, 73.99 μmol) were added, and the mixture was reacted at 110° C. for 2 hours. The mixture was cooled and concentrated under reduced pressure to give a crude product. The crude product was separated by column chromatography (methanol/ethyl acetate=0%-100%) to give compound 63-2. MS m/z: 623.2 [M+1]⁺.

### Step 3: Synthesis of a Trifluoroacetate of Compound 63

Compound 63-2 (218 mg, 350.10 μmol) was dissolved in DCM (5 mL) under nitrogen. TfOH (3.39 g, 22.60 mmol) was added, and the mixture was reacted at 40° C. for 16 hours. Aqueous ammonia (5 mL) was added to quench the reaction, and the mixture was extracted with dichloromethane:methanol=10:1 (V/V, 20 mL×4). The organic phases were combined, washed with saturated brine (20 mL), dried over anhydrous sodium sulfate, filtered and concentrated under reduced pressure to give a crude product. The crude product was separated by prep-HPLC (column: Phenomenex C18 75*30 mm*3 μm; mobile phase: [water (TFA)-acetonitrile]; acetonitrile %: 15%-45%, 8 min) to give a trifluoroacetate of compound 63. MS m/z: 473.0 [M+H]⁺; ¹H NMR (400 MHz, DMSO-d₆) δ 9.85 (s, 1H), 9.47 (s, 1H), 9.18 (s, 1H), 8.36 (s, 1H), 7.58 (s, 1H), 7.43-7.24 (m, 2H), 6.94 (s, 1H), 6.69 (d, J=6.8 Hz, 1H), 4.71 (s, 2H), 4.39 (d, J=9.6 Hz, 1H), 4.12-3.84 (m, 3H), 3.60 (d, J=4.8 Hz, 2H), 3.22-3.18 (m, 1H), 3.06 (d, J=0.8 Hz, 1H), 2.71-2.59 (m, 1H).

### Example 64

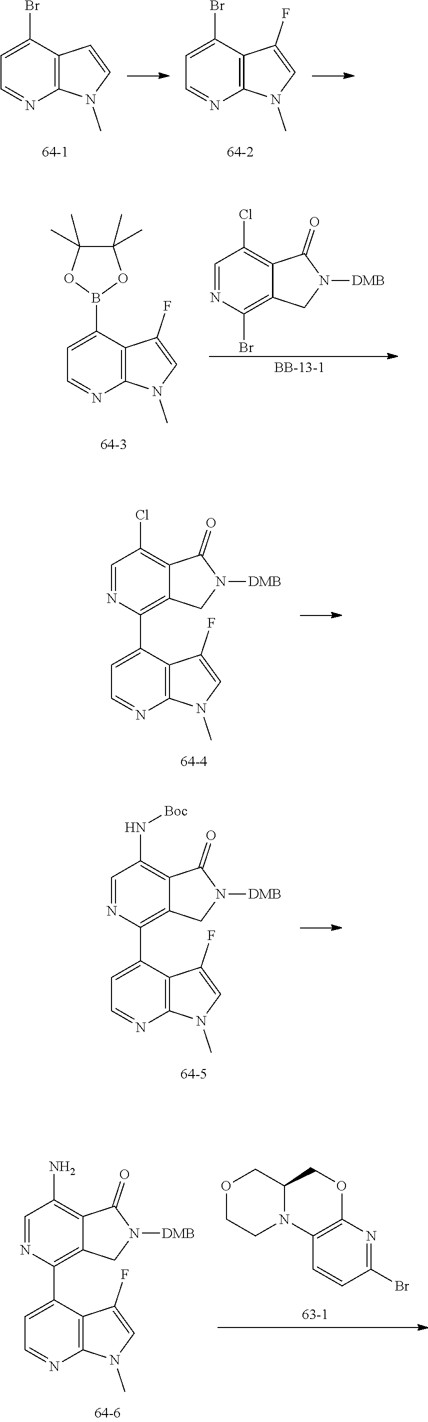

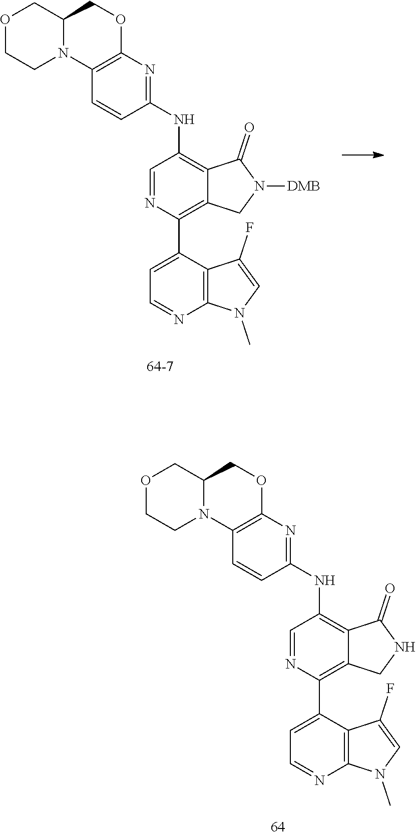

### Step 1: Synthesis of Compound 64-2

Compound 64-1 (8.5 g, 40.27 mmol) was dissolved in DMF (85 mL) under nitrogen. Select-F (14.27 g, 40.27 mmol) was added, and the mixture was stirred at 60° C. for 16 hours. The mixture was poured into water (300 mL). The aqueous phase was extracted with MTBE (50 mL×4). The organic phases were combined, dried over anhydrous sodium sulfate, filtered and concentrated to give a crude product. The crude product was separated by column chromatography (PE:EA=100:0-4:1) to give compound 64-2. MS m/z: 229.1, 231.1 [M+1]⁺.

### Step 2: Synthesis of Compound 64-3

Compound 64-2 (1 g, 4.37 mmol) was dissolved in 1,4-dioxane (20 mL) under nitrogen. Bis(pinacolato)diboron (1.66 g, 6.55 mmol), Pd(dppf)Cl₂·CH₂Cl₂ (356.53 mg, 436.59 μmol) and potassium acetate (1.29 g, 13.10 mmol) were added. The mixture was stirred at 85° C. for 16 hours. The mixture was poured into water (50 mL), and the mixture was extracted with EA (30 mL×3). The organic phases were combined, dried over anhydrous sodium sulfate, filtered and concentrated to give a crude product. The crude product was separated by column chromatography (PE:EA=100:0-2:1) to give compound 64-3. MS m/z: 195.2 [M-81]⁺; ¹H NMR (400 MHz, DMSO-d₆) δ 8.35 (d, J=4.4 Hz, 1H), 7.41 (d, J=4.4 Hz, 1H), 7.01 (s, 1H), 3.84 (s, 3H), 1.41 (s, 12H).

### Step 3: Synthesis of Compound 64-4

Compound 64-3 (360 mg, 1.30 mmol) and compound BB-13-1 (518.46 mg, 1.30 mmol) were dissolved in 1,4-dioxane (20 mL) under nitrogen. Pd(dppf)Cl₂ (190.80 mg, 260.76 μmol) and an aqueous solution (4 mL) of K₃PO₄ (830.28 mg, 3.91 mmol) were added, and the mixture was stirred at 60° C. for 2 hours. The mixture was filtered, and the filter cake was washed with EA (20 mL×3). The organic phases were combined, dried over anhydrous sodium sulfate, filtered and concentrated to give a crude product. The crude product was separated by column chromatography (DCM:MeOH=100:0-95:5) to give compound 64-4. MS m/z: 467.1 [M+1]⁺.

### Step 4: Synthesis of Compound 64-5

Compound 64-4 (120 mg, 257.02 μmol) and BocNH₂ (60.22 mg, 514.04 μmol) were dissolved in 1,4-dioxane (10 mL) under nitrogen, and K₃PO₄ (163.67 mg, 771.06 μmol) was added. The pH of the system was tested to be greater than 9. Pd₂(dba)₃ (47.07 mg, 51.40 μmol) and RuPhos (35.98 mg, 77.11 μmol) were added, and the mixture was stirred at 110° C. for 3 hours. The mixture was filtered, and the filter cake was washed with EA (20 mL×3). The organic phases were combined and concentrated to give a crude product. The crude product was separated by a preparative chromatography plate (EA) to give compound 64-5. MS m/z: 548.2 [M+1]⁺.

### Step 5: Synthesis of Compound 64-6

Compound 64-5 (55 mg, 100.44 μmol) was dissolved in DCM (2 mL). TFA (25.35 mmol, 1.88 mL) was added, and the mixture was stirred at 15° C. for 2 hours. The mixture was poured into a saturated sodium carbonate solution (20 mL), and the pH of the system was greater than 8. The aqueous phase was extracted with DCM:MeOH=5:1 (10 mL×3). The organic phases were combined, dried over anhydrous sodium sulfate, and filtered. The filtrate was concentrated to give compound 64-6. MS m/z: 448.0 [M+1]⁺.

### Step 6: Synthesis of Compound 64-7

Compound 64-6 (45 mg, 100.57 μmol) and compound 63-1 (27.26 mg, 100.57 mol) were dissolved in 1,4-dioxane (5 mL), and K₃PO₄ (64.04 mg, 301.70 μmol) was added. Then, Pd₂(dba)₃ (18.42 mg, 20.11 μmol) and RuPhos (14.08 mg, 30.17 μmol) were added, and the mixture was stirred at 110° C. under nitrogen for 3 hours. The mixture was filtered, and the filter cake was washed with ethyl acetate (20 mL). The organic phases were combined and concentrated to give a crude product. The crude product was separated by a preparative chromatography plate (DCM:MeOH=20:1) to give compound 64-7. MS m/z: 638.2 [M+1]⁺.

### Step 7: Synthesis of Compound 64

Compound 64-7 (50 mg, 78.41 μmol) was dissolved in TfOH (53.67 mmol, 4.75 mL), and the mixture was stirred at 40° C. for 1 hour. The mixture was poured into 5% dilute aqueous ammonia (20 mL), and the aqueous phase was extracted with DCM:MeOH=10:1 (10 mL×3). The organic phases were combined, dried over anhydrous sodium sulfate, and filtered. The filtrate was concentrated to give a crude product. The crude product was separated by prep-HPLC (column: Phenomenex C18 75*30 mm*3 μm; mobile phase: [water (0.1% TFA)-acetonitrile]; acetonitrile %: 15%-45%) to give a trifluoroacetate of compound 64. MS m/z: 488.0 [M+H]⁺; ¹H NMR (400 MHz, DMSO-d₆) δ 9.78 (s, 1H), 9.34 (s, 1H), 9.08 (s, 1H), 8.38 (d, J=4.8 Hz, 1H), 7.59 (s, 1H), 7.36 (d, J=4.8 Hz, 1H), 7.31 (d, J=8.4 Hz, 1H), 6.68 (d, J=8.4 Hz, 1H), 4.53 (s, 2H), 4.39-4.36 (m, 1H), 4.05-4.00 (m, 1H), 3.89-3.88 (m, 1H), 3.87-3.86 (m, 1H), 3.82 (s, 3H), 3.60-3.45 (m, 3H), 3.22-3.17 (m, 1H), 2.66-2.64 (m, 1H).

### Example 65

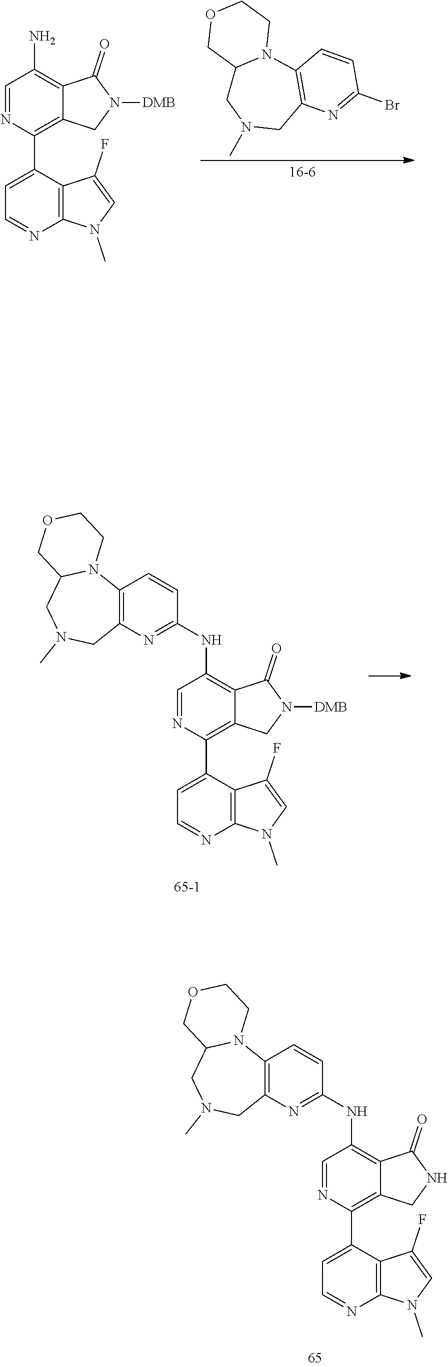

### Step 1: Synthesis of Compound 65-1

Compound 16-6 (70 mg, 234.76 μmol) and compound 64-6 (105.05 mg, 234.76 mol) were dissolved in 1,4-dioxane (5 mL), and K₃PO₄ (149.50 mg, 704.28 μmol) was added. Pd₂(dba)₃ (42.99 mg, 46.95 μmol) and RuPhos (32.86 mg, 70.43 μmol) were added, and the mixture was stirred at 110° C. under nitrogen for 3 hours. The mixture was filtered, and the filter cake was washed with EA (10 mL×2). The organic phases were combined and concentrated to give a crude product. The crude product was separated by a preparative chromatography plate (DCM:MeOH=10:1) to give compound 65-1. MS m/z: 665.3 [M+1]⁺.

### Step 2: Synthesis of Compound 65

Compound 65-1 (60 mg, 90.26 μmol) was dissolved in DCM (2 mL). TfOH (1.61 g, 10.76 mmol) was added, and the mixture was stirred at 40° C. for 16 hours. The mixture was poured into 5% dilute aqueous ammonia (20 mL), and the mixture was extracted with DCM:MeOH=10:1 (10 mL×3). The organic phases were combined, dried over anhydrous sodium sulfate, filtered and concentrated to give a crude product. The crude product was separated by prep-HPLC (column: Phenomenex C18 80*30 mm*3 μm; mobile phase: [water (0.1% TFA)-acetonitrile]; acetonitrile %: 10%-40%) to give a trifluoroacetate of compound 65. MS m/z: 515.1[M+H]⁺; ¹H NMR (400 MHz, DMSO-d₆) δ 9.98-9.96 (m, 1H), 9.66-9.60 (m, 1H), 9.16 (s, 1H), 8.40 (d, J=4.8 Hz, 1H), 7.61-7.58 (m, 2H), 7.37-7.36 (m, 1H), 7.22-7.17 (m, 1H), 4.75-7.73 (m, 1H), 4.59-4.55 (m, 3H), 3.83 (s, 3H), 3.71-3.60 (m, 3H), 3.35-2.99 (m, 6H), 2.98-2.97 (m, 3H).

### Example 66

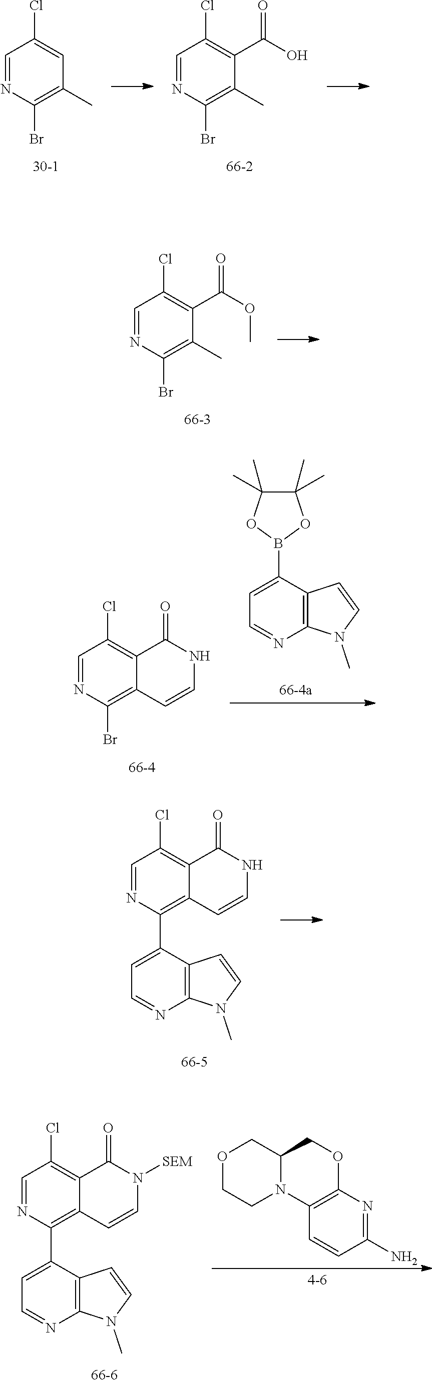

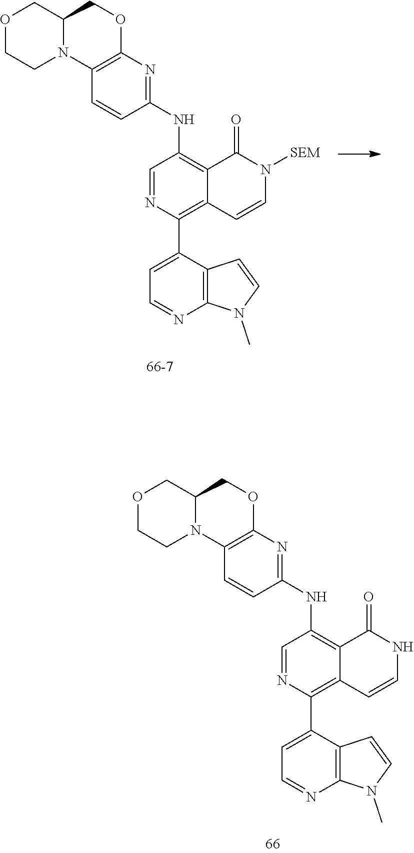 [SMILES: Cn1ccc2c(-c3ncc(Nc4ccc5c(n4)OC[C@@H]4COCCN54)c4c(=O)n(COCC[Si](C)(C)C)ccc34)ccnc21 ; Cn1ccc2c(-c3ncc(Nc4ccc5c(n4)OC[C@@H]4COCCN54)c4c(=O)[nH]ccc34)ccnc21]

### Step 1: Synthesis of Compound 66-2

Compound 30-1 (38.5 g, 186.47 mmol) was dissolved in THF (500 mL) under nitrogen. LDA (2 M, 111.88 mL) was added at −78° C., and the mixture was stirred for 1 hour. Then, C02 was introduced until the system no longer had obvious temperature changes. The mixture was slowly warmed to 25° C. and reacted for 2 hours. The mixture was poured into water (1000 mL), and the resulting mixture was extracted with EA (200 mL×3). The pH of the aqueous phase was adjusted to 1 with 2 M hydrochloric acid. The mixture was extracted with DCM (200 mL×3). The organic phases were combined, dried over anhydrous sodium sulfate, filtered and concentrated to give compound 66-2.

### Step 2: Synthesis of Compound 66-3

Compound 66-2 (26 g, 103.80 mmol) was dissolved in DMF (300 mL) under nitrogen. K₂CO₃ (43.04 g, 311.41 mmol) and Mel (29.47 g, 207.60 mmol) were added, and the mixture was stirred at 15° C. for 2 hours. The mixture was poured into water (3000 mL), and the resulting mixture was extracted with EA (1000 mL×3). The organic phases were combined, washed with semi-saturated brine (500 mL×2), dried over anhydrous sodium sulfate, filtered and concentrated to give a crude product. The crude product was separated by column chromatography (EA/PE=0%-10%) to give compound 66-3.

### Step 3: Synthesis of Compound 66-4

Compound 66-3 (7 g, 26.46 mmol) was dissolved in 1-methyl-2-pyrrolidone (70 mL) under nitrogen. 1,3,5-triazine (4.29 g, 52.93 mmol) was slowly added dropwise at 0° C., and the mixture was stirred at 0° C. for 0.5 hours. Potassium tert-butoxide (5.94 g, 52.93 mmol) was added, and the mixture was heated to 110° C. and stirred for 1 hour. The mixture was poured into water (100 mL), and the resulting mixture was extracted with EA (30 mL×3). The organic phases were combined, dried over anhydrous sodium sulfate, and concentrated to give a crude product. The crude product was separated by column chromatography (EA/PE=0%-10%-MeOH/EA=0%-5%) to give compound 66-4. MS m/z: 258.7, 260.7 [M+1]⁺; ¹H NMR (400 MHz, DMSO-d₆) δ 11.98 (s, 1H), 8.40-8.36 (m, 1H), 6.63 (d, J=7.6 Hz, 1H).

### Step 4: Synthesis of Compound 66-5

Compound 66-4 (2.5 g, 6.74 mmol) was dissolved in 1,4-dioxane (30 mL) and water (6 mL) under nitrogen. Compound 66-4a (1.74 g, 6.74 mmol), potassium phosphate (4.29 g, 20.23 mmol), and Pd(dppf)Cl₂·CH₂Cl₂ (550.75 mg, 674.41 μmol) were added. The mixture was stirred at 90° C. for 1 hour. The mixture was poured into water (200 mL), and the resulting mixture was extracted with EA (50 mL×3). The organic phases were combined, dried over anhydrous sodium sulfate, and concentrated under reduced pressure to give a crude product. The crude product was separated by column chromatography (EA/PE=10%-100%) to give compound 66-5. MS m/z: 310.8 [M+1]⁺.

### Step 5: Synthesis of Compound 66-6

Compound 66-5 (1.5 g, 4.83 mmol) was dissolved in tetrahydrofuran (5 mL) under nitrogen. 1,8-diazabicyclo[5.4.0]undec-7-ene (3.67 g, 24.14 mmol, 3.64 mL) was slowly added dropwise at 0° C., and the mixture was stirred at 0° C. for 0.5 hours. 2-(trimethylsilyl)ethoxymethyl chloride (3.22 g, 19.31 mmol) was slowly added, and the mixture was warmed to 15° C. and stirred for 16 hours. The mixture was poured into water (100 mL), and the resulting mixture was extracted with EA (30 mL×3). The organic phases were combined, dried over anhydrous sodium sulfate, and vacuum filtered. The filtrate was concentrated under reduced pressure to give a crude product. The crude product was separated by column chromatography (EA/PE=10%-50%) to give compound 66-6. MS m/z: 441.0 [M+1]⁺; ¹H NMR (400 MHz, DMSO-d₆) δ 8.76 (s, 1H), 8.49 (d, J=4.8 Hz, 1H), 7.27-7.23 (m, 3H), 6.50 (d, J=7.6 Hz, 1H), 6.27 (d, J=3.2 Hz, 1H), 5.41 (s, 2H), 3.97 (s, 3H), 3.71-3.66 (m, 2H), 0.99-0.95 (m, 2H), 0.01 (s, 9H).

### Step 6: Synthesis of Compound 66-7

Compound 66-6 (100 mg, 226.76 μmol) was dissolved in 1,4-dioxane (2 mL) under nitrogen. Compound 4-6 (46.99 mg, 226.76 μmol), potassium phosphate (144.40 mg, 680.28 μmol), Ruphos (21.16 mg, 45.35 μmol), and Pd₂(dba)₃ (20.76 mg, 22.68 μmol) were added. The mixture was stirred at 110° C. for 2 hours. The mixture was cooled to room temperature and filtered. The residue was washed with DCM (20 mL). The filtrate was concentrated under reduced pressure. The resulting crude product was separated by column chromatography (MeOH/EA=0%-2%) to give compound 66-7. MS m/z: 612.2 [M+1]⁺.

### Step 7: Synthesis of Compound 66

Compound 66-7 (80 mg, 130.77 μmol) was dissolved in TFA (26.93 mmol, 2.00 mL) under nitrogen, and the mixture was stirred at 20° C. for 16 hours. The mixture was concentrated under reduced pressure, and DCM (10 mL) was added. The pH of the mixture was adjusted to 8 with dilute aqueous ammonia (5%, 3 mL), and the layers were separated. The aqueous phase was extracted with dichloromethane (10 mL×3). The organic phases were combined, washed with saturated brine (10 mL), dried over anhydrous sodium sulfate, and filtered. The filtrate was concentrated under reduced pressure to give a crude product. The crude product was separated by prep-HPLC (column: Phenomenex C18 75*30 mm*3 μm; mobile phase: [water (0.1% TFA)-acetonitrile]; acetonitrile %: 15%-45%) to give a trifluoroacetate of compound 66. MS m/z: 482.1 [M+H]⁺; ¹H NMR (400 MHz, DMSO-d₆) δ 12.03-11.93 (m, 1H), 11.86 (s, 1H), 9.79 (s, 1H), 8.39 (d, J=4.4 Hz, 1H), 7.56 (d, J=3.6 Hz, 1H), 7.37-7.28 (m, 1H), 7.21 (d, J=3.6 Hz, 2H), 6.73-6.60 (m, 1H), 6.41-6.30 (m, 1H), 6.26 (d, J=3.6 Hz, 1H), 4.41-4.32 (m, 2H), 4.10-3.94 (m, 3H), 3.88 (s, 4H), 3.62-3.57 (m, 1H), 3.23-3.15 (m, 1H), 3.12-3.03 (m, 1H).

### Example 68

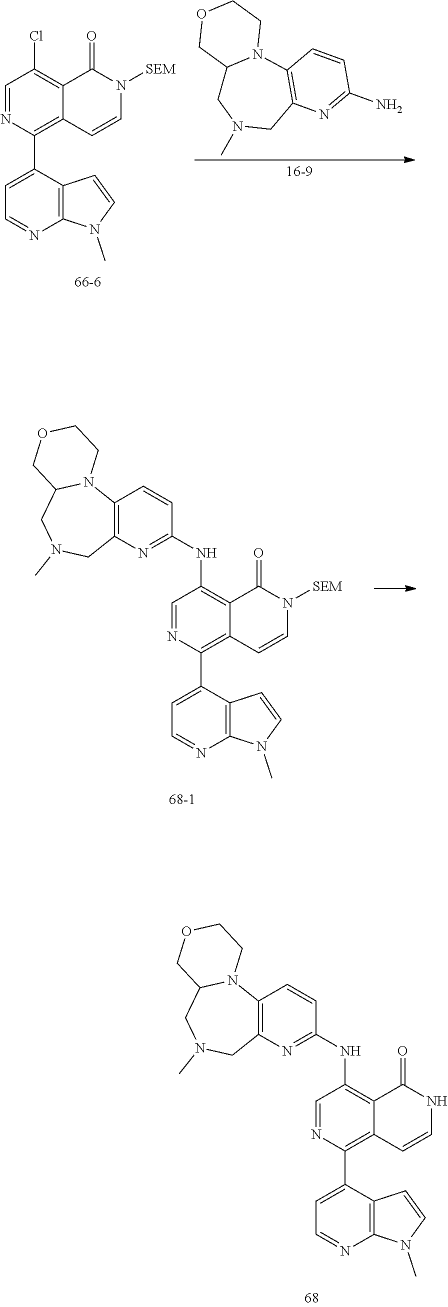 [SMILES: Cn1ccc2c(-c3ncc(Cl)c4c(=O)n(COCC[Si](C)(C)C)ccc34)ccnc21 ; CN1Cc2nc(N)ccc2N2CCOCC2C1 ; CN1Cc2nc(Nc3cnc(-c4ccnc5c4ccn5C)c4ccn(COCC[Si](C)(C)C)c(=O)c34)ccc2N2CCOCC2C1 ; CN1Cc2nc(Nc3cnc(-c4ccnc5c4ccn5C)c4cc[nH]c(=O)c34)ccc2N2CCOCC2C1]

### Step 1: Synthesis of Compound 68-1

Compound 66-6 (100 mg, 226.76 μmol) was dissolved in 1,4-dioxane (1 mL) under nitrogen. Compound 16-9 (58.44 mg, 249.43 μmol), potassium phosphate (144.40 mg, 680.28 μmol), Ruphos (21.16 mg, 45.35 μmol), and Pd₂(dba)₃ (20.76 mg, 22.68 μmol) were added. The mixture was stirred at 110° C. for 2 hours. The mixture was cooled to room temperature and vacuum filtered. The residue was washed with DCM (20 mL). The filtrate was concentrated under reduced pressure to give a crude product. The crude product was separated by flash column chromatography (MeOH/EA=0%-15%) to give compound 68-1. MS m/z: 639.3 [M+1]⁺.

### Step 2: Synthesis of a Trifluoroacetate of Compound 68

Compound 68-1 (120 mg, 187.84 μmol) was dissolved in TFA (67.31 mmol, 5 mL), and the reaction solution was stirred at 15° C. for 16 hours. The solvent was concentrated under reduced pressure. Dichloromethane (20 mL) and dilute aqueous ammonia (5%, 20 mL) were added, and the layers were separated. The aqueous phase was extracted with DCM:MeOH=10:1 (10 mL×3). The organic phases were combined, dried over anhydrous sodium sulfate, filtered and concentrated to give a crude product. The crude product was separated by prep-HPLC (column: Phenomenex C18 75*30 mm*3 μm; mobile phase: [water (0.1% TFA)-acetonitrile]; acetonitrile %: 10%-40%) to give a trifluoroacetate of compound 68. MS m/z: 509.1 [M+H]⁺. ¹H NMR (400 MHz, DMSO-d₆) δ=12.31-12.26 (m, 1H), 11.96-11.95 (m, 1H), 10.05 (s, 1H), 8.40 (d, J=5.2 Hz, 1H), 7.62-7.56 (m, 2H), 7.28-7.21 (m, 2H), 7.16-7.14 (m, 1H), 6.36-6.34 (m, 1H), 6.24-6.23 (m, 1H), 4.75-4.73 (m, 1H), 4.65-4.33 (m, 1H), 3.89 (s, 3H), 3.78-3.61 (m, 5H), 3.43-3.26 (m, 4H), 2.98 (s, 3H).

### Example 69

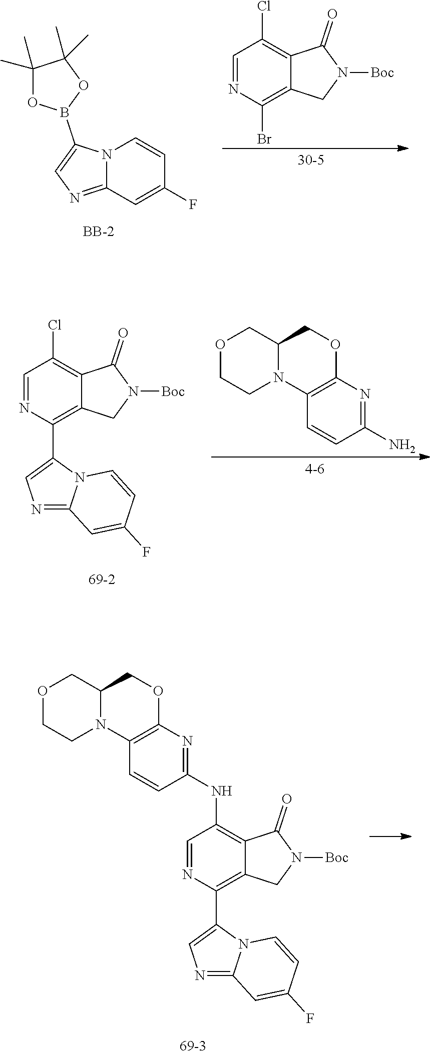

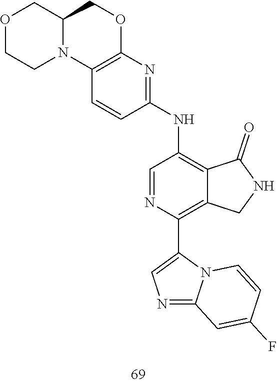 [SMILES: O=C1NCc2c(-c3cnc4cc(F)ccn34)ncc(Nc3ccc4c(n3)OC[C@@H]3COCCN43)c21]

### Step 1: Synthesis of Compound 69-2

Compound 30-5 (2.1 g, 6.04 mmol) was dissolved in 1,4-dioxane (30 mL) and water (6 mL) under nitrogen. Compound BB-2 (5.86 g, 6.04 mmol), potassium carbonate (2.50 g, 18.12 mmol), and Pd(dppf)Cl₂·CH₂Cl₂ (442.06 mg, 604.16 μmol) were added. The mixture was stirred at 70° C. for 2 hours. The mixture was cooled to room temperature and vacuum filtered. The residue was washed with DCM (20 mL). The filtrate was concentrated under reduced pressure to give a crude product. The crude product was separated by column chromatography (EA/PE=0%-100%) to give compound 69-2. MS m/z: 403.1 [M+1]⁺; ¹H NMR (400 MHz, CDCl₃) δ 10.14-9.94 (m, 1H), 8.75 (s, 1H), 8.06 (s, 1H), 7.42-7.39 (m, 1H), 7.03-6.87 (m, 1H), 5.00 (s, 2H), 1.25 (s, 9H).

### Step 2: Synthesis of Compound 69-3

Compound 69-2 (200 mg, 496.52 μmol) was dissolved in 1,4-dioxane (4 mL) under nitrogen. Compound 4-6 (102.89 mg, 496.52 μmol), potassium phosphate (316.18 mg, 1.49 mmol), Ruphos (46.34 mg, 99.30 μmol), and Pd₂(dba)₃ (45.47 mg, 49.65 μmol) were added. The mixture was reacted at 110° C. for 2 hours. The mixture was cooled to room temperature and filtered. The residue was washed with DCM (10 mL). The filtrate was concentrated under reduced pressure to give a crude product. The crude product was separated by column chromatography (MeOH/EA=0%-20%) to give compound 69-3. MS m/z: 574.2 [M+1]⁺.

### Step 3: Synthesis of Compound 69

Compound 69-3 (120 mg, 209.21 μmol) was dissolved in DCM (3 mL) under nitrogen. TFA (6.73 mmol, 0.5 mL) was added, and the mixture was reacted at 20° C. for 16 hours. The mixture was concentrated under reduced pressure to give a crude product. The crude product was separated by prep-HPLC (column: Phenomenex C18 75*30 mm*3 μm; mobile phase: [water (0.1% TFA)-acetonitrile]; acetonitrile %: 10%-40%) to give a trifluoroacetate of compound 69. MS m/z: 474.0 [M+H]⁺; ¹H NMR (400 MHz, DMSO-d₆) δ 10.10-9.97 (m, 1H), 9.87 (s, 1H), 9.44-9.26 (m, 2H), 8.27 (s, 1H), 7.85-7.75 (m, 1H), 7.38-7.25 (m, 2H), 6.68 (d, J=8.0 Hz, 1H), 4.73 (s, 2H), 4.40 (d, J=9.6 Hz, 1H), 4.09-4.01 (m, 2H), 3.98-3.89 (m, 2H), 3.64-3.56 (m, 2H), 3.22-3.17 (m, 1H), 3.12-3.02 (m, 1H).

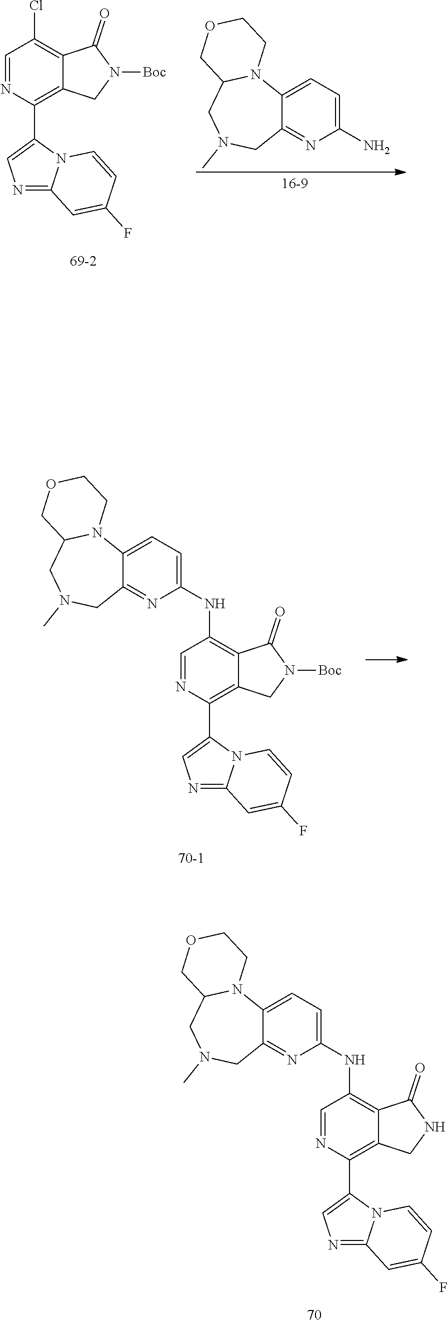 [SMILES: CC(C)(C)OC(=O)N1Cc2c(-c3cnc4cc(F)ccn34)ncc(Cl)c2C1=O ; CN1Cc2nc(N)ccc2N2CCOCC2C1 ; CN1Cc2nc(Nc3cnc(-c4cnc5cc(F)ccn45)c4c3C(=O)N(C(=O)OC(C)(C)C)C4)ccc2N2CCOCC2C1 ; CN1Cc2nc(Nc3cnc(-c4cnc5cc(F)ccn45)c4c3C(=O)NC4)ccc2N2CCOCC2C1]

### Step 1: Synthesis of Compound 70-1

Compound 69-2 (200 mg, 496.52 μmol) was dissolved in 1,4-dioxane (4 mL) under nitrogen. Compound 16-9 (116.33 mg, 496.52 μmol), potassium phosphate (316.18 mg, 1.49 mmol), Ruphos (46.34 mg, 99.30 μmol), and Pd₂(dba)₃ (45.47 mg, 49.65 μmol) were added. The mixture was reacted at 110° C. for 2 hours. The mixture was cooled to room temperature and vacuum filtered. The residue was washed with DCM (10 mL). The filtrate was concentrated under reduced pressure to give a crude product. The crude product was separated by column chromatography (MeOH/DCM=0%-10%) to give compound 70-1. MS m/z: 601.2 [M+1]⁺.

### Step 2: Synthesis of Compound 70

Compound 70-1 (110 mg, 183.14 μmol) was dissolved in DCM (3 mL) under nitrogen, and TFA (6.73 mmol, 0.5 mL) was added. The mixture was stirred at 20° C. for 16 hours, and concentrated under reduced pressure to give a crude product. The crude product was separated by prep-HPLC (column: Phenomenex C18 80*30 mm*3 μm; mobile phase: [water (0.1% TFA)-acetonitrile]; acetonitrile %: 1%-30%) to give a trifluoroacetate of compound 70. MS m/z: 501.1 [M+H]⁺; ¹H NMR (400 MHz, DMSO-d₆) δ 9.96 (s, 2H), 9.65 (s, 1H), 9.36 (s, 1H), 8.19 (s, 1H), 7.71 (d, J=8.8 Hz, 1H), 7.59 (d, J=8.4 Hz, 1H), 7.33-7.25 (m, 1H), 7.17 (d, J=8.4 Hz, 1H), 4.77 (s, 2H), 4.63-4.57 (m, 1H), 3.80-3.68 (m, 5H), 3.61-3.54 (m, 1H), 3.43-3.36 (m, 3H), 3.32-3.25 (m, 1H), 3.01 (s, 3H).

### Biological Assay Data

### Assay Example 1: In Vitro Enzyme Activity Assay of the Compounds of the Present Disclosure

Method 1: The IC₅₀ value was assayed by ³³P isotope-labeled kinase activity assay to evaluate the inhibitory ability of a test compound on human HPK1.

Enzyme buffer conditions: 50 mM Hepes (pH 7.5), 10 mM MgCl₂, 1 mM EGTA, 0.01% Brij35, 2 mM DTT.

Preparation of the mixture of kinase and substrate: the concentration of HPK1 in the reaction solution was 0.1 nM or 8.1 nM, the concentration of ATP was 10 M or 15 M, and the concentration of MBP was 0.05 mg/mL.

Assay steps: A compound was diluted 3-fold with DMSO in a dilution plate, with a final initial concentration of 10 M and 10 concentration gradient points. The compound was diluted 50-fold into the kinase reaction buffer, and the mixture was shaken on a shaker for 20 minutes. The kinase was prepared with the enzyme reaction buffer. 2 μL of kinase was added to each well of a reaction plate. 1 μL of the diluted compound in the buffer was added to each well. The plate was sealed with a sealing film, centrifuged at 1000 g for 30 seconds, and placed at room temperature for 10 minutes. The mixed solution of ATP/MBP was prepared with the enzyme reaction buffer. 2 μL of the mixed solution of ATP/MBP was added to the reaction plate. The plate was sealed with a sealing film, and centrifuged at 1000 g for 30 seconds. The mixture in the plate was reacted at room temperature for 60 minutes. 4 μL of ADP-Glo was transferred to the 384 reaction plate and centrifuged at 1000 rpm/min for 1 min. The plate was incubated at 25° C. for 40 min. 8 μL of detection solution was transferred to the 384 reaction plate, and centrifuged at 1000 rpm/min for 1 min. The plate was incubated at 25° C. for 40 min. The RLU (relative luminescence unit) signal was read with a BMG microplate reader, and the signal intensity was used to characterize the activity of the kinase. The assay results are shown in Table 1.

TABLE 1
IC₅₀ assay results of kinase activity of
the compounds of the present disclosure

| | HPK1 enzyme | ATP | |
|---|---|---|---|
| | concentration | concentration | HPK1 IC₅₀ |
| Compound No. | (nM) | (μM) | (nM) |
| Compound 2 | 8.1 | 15 | 18 |
| Compound 5 | 8.1 | 15 | 3 |
| Compound 6 | 8.1 | 15 | 8 |

Conclusion: The compounds of the present disclosure have excellent HPK1 kinase inhibitory activity.

Method 2: The IC₅₀ value was assayed by the kinase activity assay using ADP-Glo method to evaluate the inhibitory ability of a test compound on human HPK1.

Enzyme buffer conditions: 50 mM Hepes (pH 7.5), 10 mM MgCl₂, 1 mM EGTA, 0.01% Brij35, 2 mM DTT.

Preparation of the mixture of kinase and substrate: the concentration of HPK1 in the reaction solution was 0.3 nM, the concentration of ATP was 10 M, and the concentration of MBP was 0.05 mg/mL.

Assay steps: A compound was diluted 4-fold with DMSO in a dilution plate, with a final initial concentration of 10 M and 10 concentration gradient points. The compound was diluted 50-fold into the kinase reaction buffer, and the mixture was shaken on a shaker for 20 minutes. The kinase was prepared with the enzyme reaction buffer. 2 μL of kinase was added to each well of a reaction plate. 1 μL of the diluted compound in the buffer was added to each well. The plate was sealed with a sealing film, centrifuged at 1000 g for 30 seconds, and placed at room temperature for 10 minutes. The mixed solution of ATP/MBP was prepared with the enzyme reaction buffer. 2 μL of the mixed solution of ATP/MBP was added to the reaction plate. The plate was sealed with a sealing film, and centrifuged at 1000 g for 30 seconds. The mixture in the plate was reacted at room temperature for 60 minutes. 4 μL of ADP-Glo was transferred to the 384 reaction plate, and centrifuged at 1000 rpm/mi for 1 min. The plate was incubated at 25° C. for 40 min. 8 μL of detection solution was transferred to the 384 reaction plate, and centrifuged at 1000 rpm/min for 1 min. The plate was incubated at 25° C. for 40 min. The RLU (relative luminescence unit) signal was read with a BMG microplate reader, and the signal intensity was used to characterize the activity of the kinase.

The kinase activity data were expressed as the comparison of the kinase activity of a group with a test compound and the kinase activity of the blank group (containing only DMSO). The IC₅₀ value was obtained by curve fitting using Prism software (GraphPad7.0). The assay results are shown in Table 2.

TABLE 2
IC₅₀ assay results of kinase activity of
the compounds of the present disclosure

| | HPK1 | | |
|---|---|---|---|
| | enzyme | ATP | |
| | concen- | concen- | HPK1 |
| | tration | tration | IC₅₀ |
| Compound No. | (nM) | (μM) | (nM) |
| Compound 4 | 0.1 | 10 | 0.62 |
| Compound 8 | 0.1 | 10 | 2.0 |
| Compound 9 | 0.1 | 10 | 0.7 |
| Trifluoroacetate of compound 11 | 0.3 | 10 | 0.6 |
| Trifluoroacetate of compound 12 | 0.3 | 10 | 0.9 |
| Trifluoroacetate of compound 13 | 0.3 | 10 | 2.1 |
| Trifluoroacetate of compound 14 | 0.3 | 10 | 1.4 |
| Trifluoroacetate of compound 15 | 0.3 | 10 | 1.7 |
| Trifluoroacetate of compound 16 | 0.3 | 10 | 0.8 (n = 2) |
| Trifluoroacetate of compound 17 | 0.3 | 10 | 1.3 |
| Trifluoroacetate of compound 18 | 0.3 | 10 | 0.2 |
| Trifluoroacetate of compound 20 | 0.3 | 10 | 0.3 |
| Trifluoroacetate of compound 21 | 0.3 | 10 | 0.06 |
| Trifluoroacetate of compound 22 | 0.3 | 10 | 0.17 |
| Trifluoroacetate of compound 23 | 0.3 | 10 | 0.02 |
| Trifluoroacetate of compound 24-3 | 0.3 | 10 | 0.33 (n = 2) |
| Trifluoroacetate of compound 25 | 0.3 | 10 | 0.2 |
| Trifluoroacetate of compound 29 | 0.3 | 10 | 0.74 |
| Trifluoroacetate of compound 32 | 0.3 | 10 | 0.04 |
| Trifluoroacetate of compound 33 | 0.3 | 10 | 0.3 |
| Trifluoroacetate of compound 24 | 0.05 | 10 | 0.31 |
| Trifluoroacetate of compound 40 | 0.05 | 10 | 0.43 |
| Trifluoroacetate of compound 54-2 | 0.3 | 10 | 0.15 (n = 2) |
| Trifluoroacetate of compound 54 | 0.05 | 10 | 0.41 |
| Trifluoroacetate of compound 57 | 0.05 | 10 | 0.38 |
| Trifluoroacetate of compound 63 | 0.3 | 10 | 0.9 |
| Trifluoroacetate of compound 68 | 0.3 | 10 | 1.4 |
| Trifluoroacetate of compound 70 | 0.3 | 10 | 0.9 |

Conclusion: The compounds of the present disclosure have excellent HPK1 kinase inhibitory activity.

### Assay Example 2: IL-2 Assay of Jurkat Cells Treated with the Compounds of The Present Disclosure

The concentration of Jurkat cells growing in the logarithmic phase was adjusted to 1×10⁶/mL. 100 μL/well of the cells was taken and plated in a 96-well round-bottom plate coated with CD3 antibodies. The cells were treated with different concentrations of the compounds with the addition of CD28 antibodies for 48 hours. The supernatant of the cell culture was collected, diluted 5-fold, and added to an ELISA plate coated with IL-2. The enzyme-linked immunosorbent assay was performed according to the kit instructions (Dakewe, 1110202). OD570 and OD450 were read with Envision, and the data were analyzed by Prism. The assay results are shown in Table 3.

TABLE 3
IL-2 assay results of Jurkat cells treated with
the compounds of the present disclosure

| Compound No. | IL-2 EC₅₀ (μM) |
|---|---|
| Compound 3 | 0.19 |
| Compound 5 | 0.71 |
| Compound 6 | 0.32 |

Conclusion: The compounds of the present disclosure have an effect of significantly increasing the IL-2 level of Jurkat cells.

### Assay Example 3: In Vitro Enzyme Activity Assay of the Compounds of the Present Disclosure

The IC₅₀ value was assayed by the kinase activity assay using ADP-Glo method to evaluate the inhibitory ability of a test compound on human GLK.

Enzyme buffer conditions: 50 mM Hepes (pH 7.5), 10 mM MgCl₂, 1 mM EGTA, 0.01% Brij35, 2 mM DTT.

Preparation of the mixture of kinase and substrate: the concentration of GLK in the reaction solution was 8 nM, the concentration of ATP was 20 M, and the concentration of MBP was 0.05 mg/mL.

Assay steps: A compound was diluted 4-fold with DMSO in a dilution plate, with a final initial concentration of 10 M and 10 concentration gradient points. The compound was diluted 50-fold into the kinase reaction buffer, and the mixture was shaken on a shaker for 20 minutes. The kinase was prepared with the enzyme reaction buffer. 2 μL of kinase was added to each well of a reaction plate. 1 μL of the diluted compound in the buffer was added to each well. The plate was sealed with a sealing film, centrifuged at 1000 g for 30 seconds, and placed at room temperature for 10 minutes. The mixed solution of ATP/MBP was prepared with the enzyme reaction buffer. 2 μL of the mixed solution of ATP/MBP was added to the reaction plate. The plate was sealed with a sealing film, and centrifuged at 1000 g for 30 seconds. The mixture in the plate was reacted at room temperature for 60 minutes. 4 μL of ADP-Glo was transferred to the 384 reaction plate, and centrifuged at 1000 rpm/min for 1 min. The plate was incubated at 25° C. for 40 min. 8 μL of detection solution was transferred to the 384 reaction plate, and centrifuged at 1000 rpm/min for 1 min. The plate was incubated at 25° C. for 40 min. The RLU (relative luminescence unit) signal was read with a BMG microplate reader, and the signal intensity was used to characterize the activity of the kinase.

The kinase activity data were expressed as the comparison of the kinase activity of a group with a test compound and the kinase activity of the blank group (containing only DMSO). The IC₅₀ value was obtained by curve fitting using Prism software (GraphPad7.0). The assay results are shown in Table 4.

TABLE 4
IC₅₀ assay results of kinase activity of
the compounds of the present disclosure

| | GLK enzyme | GLK | |
|---|---|---|---|
| | concentration | concentration | GLK IC₅₀ |
| Compound No. | (nM) | (μM) | (nM) |
| Compound 4 | 8 | 20 | 594 |
| Compound 8 | 8 | 20 | 154 |
| Compound 9 | 8 | 20 | 115 |
| Trifluoroacetate of | 8 | 20 | 121 |
| compound 11 | | | |
| Trifluoroacetate of | 8 | 20 | 173 |
| compound 12 | | | |
| Trifluoroacetate of | 8 | 20 | 255 |
| compound 13 | | | |
| Trifluoroacetate of | 8 | 20 | 252 |
| compound 14 | | | |
| Trifluoroacetate of | 8 | 20 | 277 |
| compound 16 | | | |
| Trifluoroacetate of | 8 | 20 | 198 |
| compound 20 | | | |

Conclusion: The compounds of the present disclosure weakly inhibit the GLK subtype in the MAP4K family and have relatively weak activity.

### Assay Example 4: Assay of the Inhibitory Activity of the Compounds of the Present Disclosure on SLP76 Phosphorylation in Jurkat Cells

30 μL of Jurkat cells (300,000 cells) were added to a round-bottom 96-well plate, and incubated in a 37° C. incubator for 1 hour. Then, 7.5 μL of a compound at different concentrations was added and incubated for 1 hour. 7.5 μL of anti-CD3 antibody was added to make the final concentration 10 μg/mL. The plate was incubated at 37° C. for 15 minutes. Then, 15 μL of 4× lysis buffer (Cisbio, 63ADK076PEG) was added, and the cells were lysed at room temperature with shaking for 1 hour. 16 μL of cell lysate was added to a 96-well plate, 4 μL of antibody detection mixture was added, and the plate was incubated overnight at room temperature. The next day, the readings at 665 nm and 615 nm were read with the EnVision multi-function microplate reader, and the ratio of the 665 nm signal to the 615 nm signal was converted to a percentage inhibition rate, i.e., inhibition rate %=(maximum value−sample value)/(maximum value−minimum value)×100. The minimum value refers to the signal value of the well without anti-CD3 antibody stimulation during the cell treatment process, and the maximum value refers to the signal value of the well with anti-CD3 antibody stimulation but no compound added during the cell treatment process. Data were analyzed by Prism and IC₅₀ value was calculated. Equation: Y=Bottom+(Top-Bottom)/(1+(IC50/X)\hat{ }HillSlope). The assay results are shown in Table 5.

TABLE 5
The results of the inhibition of SLP76 phosphorylation in
Jurkat cells of the compounds of the present disclosure

| Compound No. | IC₅₀ (μM) |
|---|---|
| Trifluoroacetate of compound 24-3 | 0.064 |
| Trifluoroacetate of compound 32 | 0.075 |
| Trifluoroacetate of compound 35 | 0.042 |
| Trifluoroacetate of compound 36 | 0.077 |
| Trifluoroacetate of compound 54-2 | 0.116 |
| Trifluoroacetate of compound 63 | 0.268 |
| Trifluoroacetate of compound 70 | 0.138 |

Conclusion: The compounds of the present disclosure have excellent inhibitory activity on the phosphorylation of SLP76 in Jurkat cells.

### Assay Example 5: In Vitro Stability in Liver Microsomes of the Compounds of the Present Disclosure

The in vitro metabolic stability of the compounds of the present disclosure in human was investigated by the metabolic stability in human liver microsomes.

Assay conditions: 1 μM of a compound was incubated with human liver microsomes supplemented with NADPH regeneration system at 37° C. for a certain period of time up to 60 minutes. The concentration of the compound in the resulting sample was assayed by LC-MS/MS method.

Assay procedure: 96-well incubation plates were named T0, T5, T15, T30, T45, T60, Blank60 and NCF60, respectively. The reaction time corresponding to the incubation plates were 0, 5, 15, 30, 45 and 60 minutes, respectively. No test sample or control compound was added to the Blank60 plate, and samples were taken after 60 minutes of incubation. Potassium phosphate buffer was used instead of NADPH regeneration system solution in the NCF60 plate, and the plate was incubated for 60 minutes. All the samples of the time points were from a single well.

5 μL of a test sample or control sample working solution and 100 μL of microsome working solution (the concentration of liver microsome protein was 0.5 mg/mL) were added to the T0, T5, T15, T30, T45, T60 and NCF60 plates, respectively. Only microsome working solution was added to the Blank60 plate. Then, the Blank60, T5, T15, T30, T45 and T60 plates, except TO and NCF60 plates, were placed in a 37° C. water bath, and preincubated for about 10 minutes.

180 μL of stop solution (the stop solution was a solution of tolbutamide (100 ng/mL) in acetonitrile and methanol (95:5, V/V)) was firstly added to the sample of TO plate, and then the NADPH regeneration system working solution was added.

50 μL of potassium phosphate buffer was added to each well of the NCF60 plate, and the plate was incubated for 60 minutes.

After the pre-incubation of Blank60, T5, T15, T30, T45 and T60 plates, 44 μL of NADPH regeneration system working solution was added to each sample well to initiate the reaction. Therefore, for the sample wells containing a test sample or control sample working solution, the final concentration of the reaction was 1 M, the concentration of liver microsomes was 0.5 mg/mL, and the final concentrations of DMSO and acetonitrile in the reaction system were 0.01% (v/v) and 0.99% (v/v), respectively.

After being incubated for an appropriate time (e.g., 5, 15, 30, 45, and 60 min), 180 μL of stop solution was added to each well of the test sample and control sample of Blank60, T5, T15, T30, T45, T60, and NCF60 plates, respectively, to terminate the reaction.

All sample plates were shaken and centrifuged for 10 min. 80 μL of the supernatant of a test sample was taken and diluted into 240 μL of a mixture of acetonitrile and water (1:9, V/V) containing 0.1% formic acid for LC-MS/MS analysis.

By calculating the corresponding time point and the remaining percentage of a compound, the half-life T_{1/2} and clearance rate CLᵢₙₜ₍ₗᵢᵥₑᵣ₎ of the compound in human liver microsome metabolism were obtained. The assay results are shown in Table 6.

$${C}_{t} = {C}_{0} \cdot {e}^{- {k}_{e} \cdot t} \\ when \\ {C}_{t} = \frac{1}{2} {C}_{0} , \\ {T}_{1 / 2} = \frac{Ln 2}{{k}_{e}} = \frac{0.693}{{k}_{e}} \\ {CL}_{i n t ( mic )} = \frac{0.693}{In vitro {T}_{1 / 2}} \cdot \frac{1}{mg / mL microsomal protein in reaction system} \\ {CL}_{i n t ( liver )} = {CL}_{i n t ( mic )} \cdot \frac{mg microsomes}{g liver} \cdot \frac{g liver}{kg body weight}$$

TABLE 6
Results of in vitro human liver microsome metabolic stability
study of the compounds of the present disclosure

| Compound No. | CLᵢₙₜ₍ₗᵢᵥₑᵣ₎ (ml/min/kg) | T_{1/2} (minutes) |
|---|---|---|
| Compound 4 | 35 | 36 |
| Compound 8 | 37 | 34 |
| Compound 9 | 39 | 32 |
| Trifluoroacetate of | 64 | 20 |
| compound 11 | | |
| Trifluoroacetate of | 54 | 23 |
| compound 14 | | |
| Trifluoroacetate of | 63 | 20 |
| compound 20 | | |
| Trifluoroacetate of | 58 | 21 |
| compound 24-3 | | |
| Trifluoroacetate of | 58 | 21 |
| compound 24 | | |
| Trifluoroacetate of | 68 | 18 |
| compound 40 | | |
| Trifluoroacetate of | 29 | 43 |
| compound 35 | | |
| Trifluoroacetate of | 18 | 68 |
| compound 36 | | |
| Trifluoroacetate of | 10 | 125 |
| compound 46 | | |
| Trifluoroacetate of | 27 | 47 |
| compound 47 | | |
| Trifluoroacetate of | 40 | 32 |
| compound 54-2 | | |
| Trifluoroacetate of | 66 | 19 |
| compound 54 | | |
| Trifluoroacetate of | 49 | 25 |
| compound 57 | | |
| Trifluoroacetate of | 28 | 44 |
| compound 63 | | |
| Trifluoroacetate of | 41 | 30 |
| compound 66 | | |
| Trifluoroacetate of | 27 | 46 |
| compound 69 | | |

Conclusion: The compounds of the present disclosure have good stability in human liver microsomes.

### Assay Example 6: Pharmacokinetic Evaluation in Mice

Assay procedure: 10% DMSO+20% PEG400+70% (30% SBE-P3-CD aqueous solution) (the trifluoroacetate of compound 4, the trifluoroacetate of compound 9) or 10% SBE-β-CD aqueous solution (the trifluoroacetate of compound 24-3) was injected into female Balb/c mice (overnight fasting, 7-9 weeks old) via the tail vein. Female Balb/c mice (overnight fasting, 7-9 weeks old) were gavaged with 10% DMSO+20% PEG400+70% (30% SBE-β-CD aqueous solution (the trifluoroacetate of compound 4, the trifluoroacetate of compound 9) or 10% SBE-P3-CD aqueous solution (the trifluoroacetate of compound 24-3) at dosages shown in Table 7. About 30 μL of blood of the two groups of animals was collected from the jugular vein at 0.0833, 0.25, 0.5, 1.0, 2.0, 4.0, 8.0, and 24 hours after the administration, and from the tail vein at 0.25, 0.5, 1.0, 2.0, 4.0, 8.0, and 24 hours after the administration, respectively, and placed in an anticoagulant tube with EDTA-K2 added. The tube was centrifuged to separate the plasma. The plasma concentration was assayed by LC-MS/MS. The relevant pharmacokinetic parameters were calculated with WinNonlin™ Version 6.3 (Pharsight, Mountain View, CA) pharmacokinetic software using the non-compartmental linear logarithmic trapezoidal method.

The assay results are shown in Table 7:

TABLE 7
Pharmacokinetic data

| | Trifluoroacetate of | | Trifluoroacetate of | | Trifluoroacetate of | |
|---|---|---|---|---|---|---|
| | compound 4 | | compound 9 | | compound 24-3 | |
| | IV | PO | IV | PO | IV | PO |
| PK parameters | 0.2 mg/kg | 1 mg/kg | 2 mg/kg | 75 mg/kg | 2 mg/kg | 75 mg/kg |
| Concentration of | 0.04 | 0.1 | 0.4 | 7.5 | 0.4 | 7.5 |
| compound in | | | | | | |
| solvent | | | | | | |
| C₀ (nM) or Cₘₐₓ | 322 | 613 | 6060 | 39802 | 2233 | 4330 |
| (nM) | | | | | | |
| Tₘₐₓ (h) | — | 1 | — | 0.8 | — | 1 |
| T_{1/2} (h) | 0.8 | 1 | 0.3 | 0.8 | 0.4 | 0.9 |
| Vdₛₛ (L/kg) | 1.9 | — | 0.8 | — | 3.2 | — |
| Cl (mL/min/kg) | 35 | — | 36 | — | 145 | — |
| Tₗₐₛₜ (h) | 4 | 8 | 2 | 8 | 4 | 8 |
| AUC₀₋ₗₐₛₜ (nM · h) | 191 | 924 | 1928 | 107697 | 630 | 9936 |
| AUC_{0-inf} (nM · h) | 204 | 953 | 1949 | 107965 | 633 | 9971 |
| F (%) | — | 97% | — | 148% | — | 42% |

Note:
“—” indicates that the parameter cannot be obtained by calculation; C₀ represents initial concentration; Cₘₐₓ represents peak concentration; Tₘₐₓ represents time to peak concentration; T_{1/2} represents half-life of elimination; V_{dss} represents apparent distribution volume at steady state; Cl represents total clearance rate; Tₗₐₛₜ represents the last time point at which drug concentration can be quantified; AUC₀₋ₗₐₛₜ represents the area under the plasma concentration-time curve from time 0 to the last quantifiable time point; AUC_{0-inf} represents the area under the plasma concentration-time curve from time 0 to infinity; F (%) represents bioavailability, which is calculated using AUC₀₋ₗₐₛₜ.
Conclusion: The compounds of the present disclosure have good in vivo metabolic stability, excellent oral absorption drug exposure and good oral absorption bioavailability.

### Assay Example 7: Evaluation of the Permeability of the Compounds of the Present Disclosure

Cell line: The MDR1-MDCK II cell line authorized by Piet Borst's laboratory at the Netherlands Cancer Research Institute was used in this assay as an in vitro model for the permeability evaluation assay. The cells were inoculated in a Transwell-96 well cell plate at a density of 2.3×10⁵ cells/cm², and cultured in a carbon dioxide incubator for 4-7 days before use in the transport assay.

### Assay Conditions:

The administration concentration of the test sample was 2.00 μM.

Test direction and number of parallel samples: bidirectional A-B and B-A, 2 parallels

Transport buffer (TB): HBSS solution containing 10 mM HEPES (pH 7.40±0.05)

Incubation conditions: 37±1° C., 5% CO₂, incubated for 150 minutes

Control compounds: Nadolol and metoprolol were used as low-permeability and high-permeability control compounds. Digoxin was used as a substrate of P-glycoprotein. The administration concentration of nadolol and metoprolol was 2.00 μM, and the administration concentration of digoxin was 10.0 μM.

### Monolayer Cell Membrane Integrity Test:

After the transport assay, the integrity of the MDR1-MDCK II cell layer was assayed by the Lucifer Yellow Rejection Assay. The remaining solution in the apical and basolateral wells was removed. 75 μL of TB containing 100 μM Lucifer Yellow was added to the apical well, and 250 μL of TB was added to the basolateral well. The cell plate was incubated in a cell culture incubator at 37±1° C., 5% CO₂ and saturated humidity for 30 minutes. Then, 20 μL of sample was taken from the apical well and mixed with 60 μL of TB, and 80 μL of sample was taken from the basolateral well. The relative fluorescence unit (RFU) was measured at 425/528 nm (excitation/emission) using a microplate reader.

### Sample Analysis:

In this assay, the sample analysis of the test samples and control compounds of nadolol, metoprolol, and digoxin was performed by liquid chromatography-tandem mass spectrometry (LC-MS/MS). The retention time of the analytes and internal standards, chromatogram acquisition, and chromatogram integration were processed using the software Analyst (Sciex, Framingham, MA, USA). The sample analysis was semi-quantitatively determined using the peak area of analyte and the peak area of internal standard.

### Data Analysis:

$${P}_{app} = \frac{{V}_{R}}{Area \times Time} \times \frac{{[ drug ]}_{receiver}}{{[ drug ]}_{initial , donor}} = \frac{{V}_{R}}{Area \times Time} \times \frac{{C}_{R}}{{C}_{0}} \\ Efflux Ratio = \frac{{P}_{app} ( B to A )}{{P}_{app} ( A to B )} \\ \% Solution Recovery = \frac{{C}_{R} \times {V}_{R} + {C}_{D} \times {V}_{D}}{{C}_{0} \times {V}_{D}} \times 100$$

V_{R} is the volume of the receiver end solution (0.075 mL for side A, and 0.25 mL for side B); Area is the relative surface area of the cell monolayer (0.0804 cm2); Time is the incubation time (9000 s); C₀ is the peak area ratio of the compound at the administration end; V_{D} is the volume of the administration end (0.075 mL for side A, and 0.25 mL for side B); C_{D} and C_{R} are the peak area ratios of the compound at the administration end and the receiver end, respectively.

The transmittance of Lucifer Yellow (% Lucifer Yellow) was calculated using the following formula:

$$\% Lucifer Yellow = \frac{{V}_{Basolateral} \times {RFU}_{Basolateral}}{{V}_{Apical} \times {RFU}_{Apical} + {V}_{Basolateral} \times {RFU}_{Basolateral}} \times 100$$

RFU_{Apical} and RFU_{Basolateral} are the relative fluorescence units of Lucifer Yellow at the apical and basolateral ends, respectively. V_{Apical} and V_{Basolateral} are the loading volumes at the apical and basolateral ends, respectively (0.0750 and 0.250 mL, respectively).

### Assay Results:

The results are as shown in Table 8.

TABLE 8
Summary of permeability evaluation of
the compounds of the present disclosure

| | Permeability Pₐₚₚ | | | Conclusion |
|---|---|---|---|---|
| | (10⁻⁶ cm/s) | | Efflux | of the |
| Compound | A to B | B to A | ratio | permeability |
| Compound 4 | 5.20 | 25.84 | 4.97 | Medium |
| | | | | permeability |
| Compound 8 | 1.97 | 14.16 | 7.19 | Medium |
| | | | | permeability |
| Trifluoroacetate of | 3.35 | 18.05 | 5.39 | Medium |
| compound 11 | | | | permeability |
| Trifluoroacetate of | 4.61 | 22.49 | 4.88 | Medium |
| compound 20 | | | | permeability |
| Trifluoroacetate of | 2.31 | 28.60 | 12.38 | Medium |
| compound 24-3 | | | | permeability |
| Trifluoroacetate of | 2.94 | 21.94 | 7.46 | Medium |
| compound 38 | | | | permeability |

Note:
Low permeability: Papp ≤ 1.0 (×10⁻⁶ cm/s); Medium permeability 1.0 < Papp < 5.5 (×10⁻⁶ cm/s); High permeability: Papp ≥ 5.5 (×10⁻⁶ cm/s).
Conclusion: The compounds of the present disclosure exhibit excellent membrane permeability in the cell membrane permeability study.

1. A compound represented by formula (P), a stereoisomer or a pharmaceutically acceptable salt thereof, 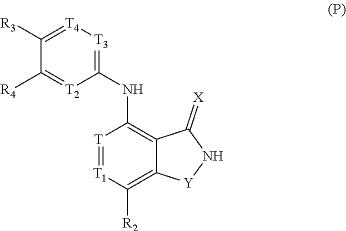
wherein,
X is selected from O and S;
Y is selected from C(R₈)₂ and C(R₈)═C(R₈);
T is selected from CH and N;
T₁ is selected from CH and N;
T₂ and T₃ are each independently selected from CH, CF and N;
T₄ is selected from CR₆ and N;
R₂ is heteroaryl having 5-membered ring fused to 6-membered ring, wherein the heteroaryl having 5-membered ring fused to 6-membered ring is optionally substituted with 1, 2 or 3 Rₐ;
R₃ and R₄ are taken together with the carbon atoms to which they are attached to form 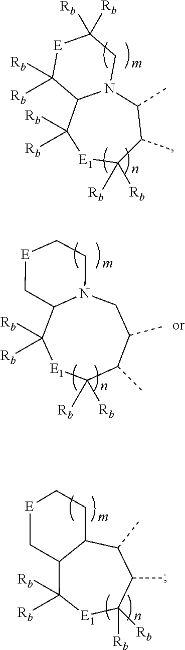
E and E₁ are each independently selected from —C(R₇)₂—, —O— and —N(R₅)—;
n and m are each independently selected from 0 and 1;
R₅ is selected from H and C₁₋₃ alkyl, wherein the C₁₋₃ alkyl is optionally substituted with 1, 2 or 3 R;
R₆ is selected from H, halogen and —C₁₋₃ alkyl-C₁₋₃ alkylamino;
R₇ is selected from H, F, Cl, Br, I, OH, NH₂, C₁₋₃ alkyl and C₁₋₃ alkoxy;
R₈ is selected from H and D;
each Rₐ is independently selected from D, halogen, C₁₋₃ alkyl and C₁₋₃ alkoxy, wherein the C₁₋₃ alkyl and C₁₋₃ alkoxy are each independently and optionally substituted with 1, 2 or 3 R;
each R_{b} is independently selected from H, D, OH, halogen and C₁₋₃ alkyl, wherein the C₁₋₃ alkyl is optionally substituted with 1, 2 or 3 halogens;
or, two R_{b} are taken together with the carbon atom to which they are jointly attached to form C═O or cyclopropyl;
each R is independently selected from halogen and D.

2. The compound according to claim 1, a stereoisomer or a pharmaceutically acceptable salt thereof, which is selected from: 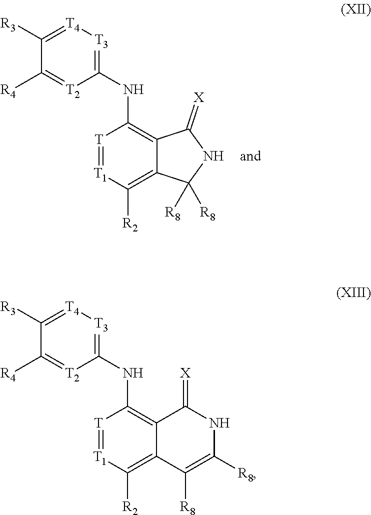
wherein,
X, T, T₁, T₂, T₃, T₄, R₂, R₃, R₄ and R₈ are as defined in claim 1.

3. The compound according to claim 1, a stereoisomer or a pharmaceutically acceptable salt thereof, wherein, each Rₐ is independently selected from D, F, CH₃ and CD₃.

4. The compound according to claim 1, a stereoisomer or a pharmaceutically acceptable salt thereof, wherein, each R_{b} is independently selected from H, D, F, OH, CH₃ and CF₃.

5. The compound according to claim 1, a stereoisomer or a pharmaceutically acceptable salt thereof, wherein, R₂ is selected from pyrrolopyridinyl and imidazopyridinyl, and the pyrrolopyridinyl and imidazopyridinyl are optionally substituted with 1, 2 or 3 Rₐ.

6. The compound according to claim 5, a stereoisomer or a pharmaceutically acceptable salt thereof, wherein, R₂ is selected from

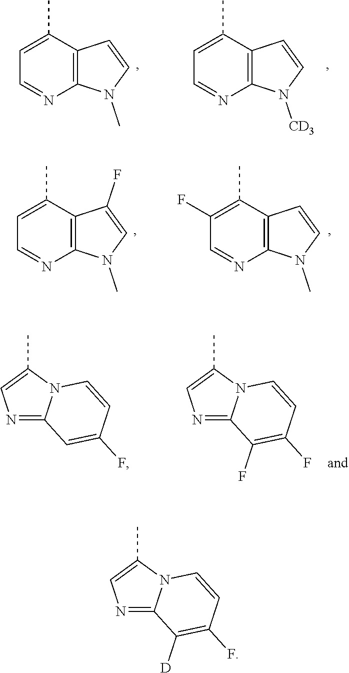

7. The compound according to claim 1, a stereoisomer or a pharmaceutically acceptable salt thereof, wherein, R₆ is selected from H, F and —CH₂—N(CH₃)₂.

8. The compound according to claim 1, a stereoisomer or a pharmaceutically acceptable salt thereof, wherein, R₅ is selected from H, CH₃, CD₃, CH₂CH₃ and CH(CH₃)₂.

9. The compound according to claim 1, a stereoisomer or a pharmaceutically acceptable salt thereof, wherein, R₇ is selected from H, F, OH, CH₃ and OCH₃.

10. The compound according to claim 1, a stereoisomer or a pharmaceutically acceptable salt thereof, wherein, E and E₁ are each independently selected from —CH₂—, —CHF—, —CF₂—, —CH(OH)—, —CH(CH₃)—, 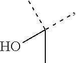
—O—, —NH—, —N(CH₃)—, —N(CD₃)-, —N(CH₂CH₃)— and

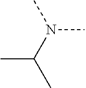

11. The compound according to claim 1, a stereoisomer or a pharmaceutically acceptable salt thereof, wherein, R₃ and R₄ are taken together with the carbon atoms to which they are attached to form

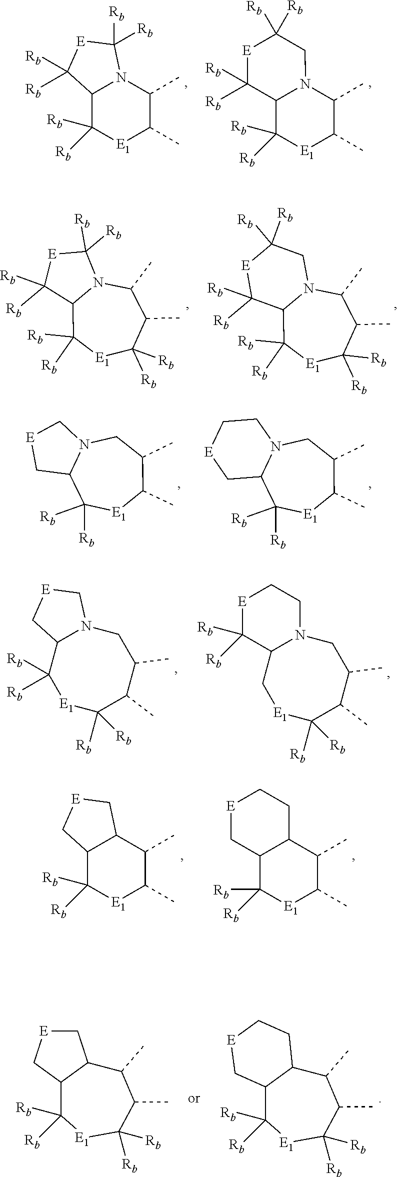

12. The compound according to claim 1, a stereoisomer or a pharmaceutically acceptable salt thereof, wherein, R₃ and R₄ are taken together with the carbon atoms to which they are attached to form 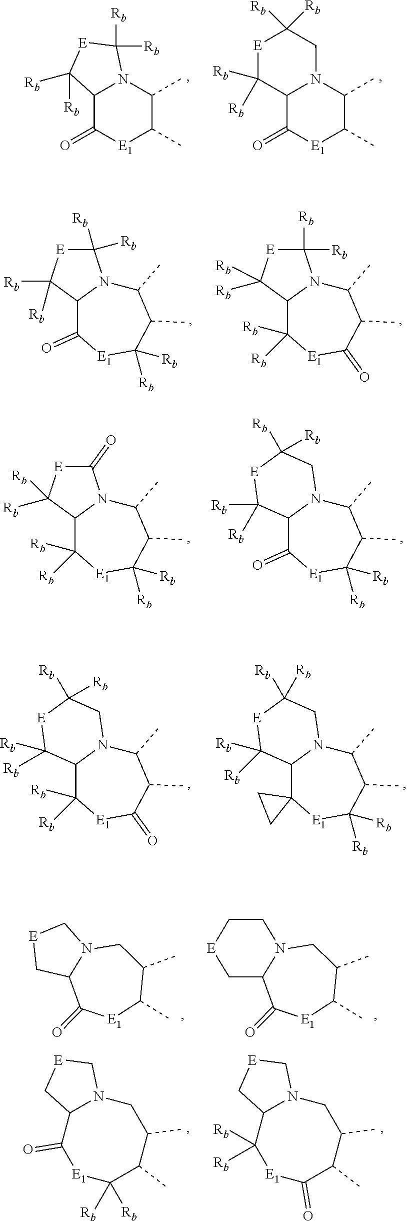 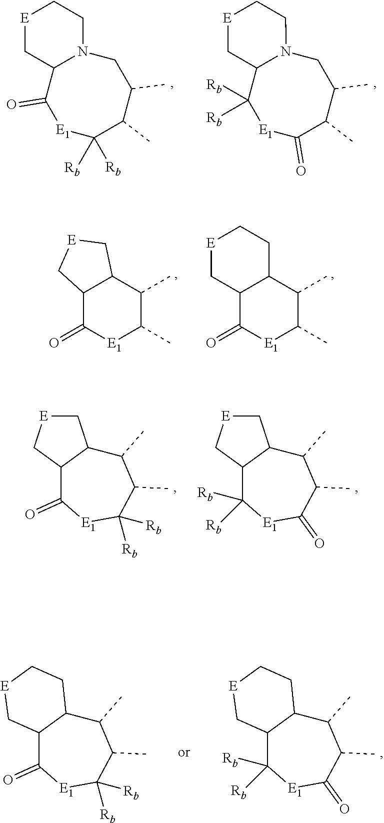
and each R_{b} is independently selected from H, D, OH, halogen and C₁₋₃ alkyl, and the C₁₋₃ alkyl is optionally substituted with 1, 2 or 3 halogens.

13. The compound according to claim 1, a stereoisomer or a pharmaceutically acceptable salt thereof, wherein, R₃ and R₄ are taken together with the carbon atoms to which they are attached to form

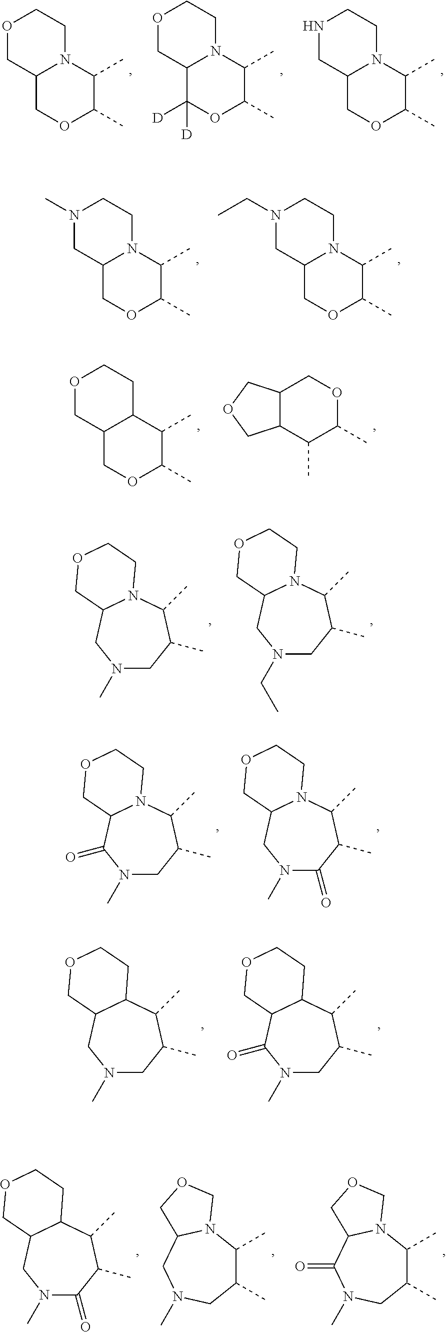

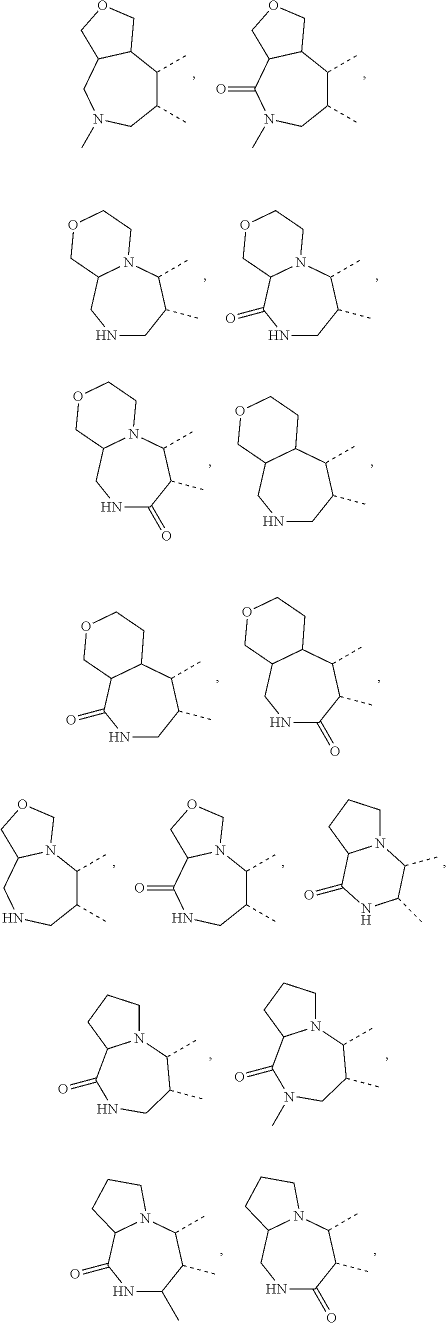

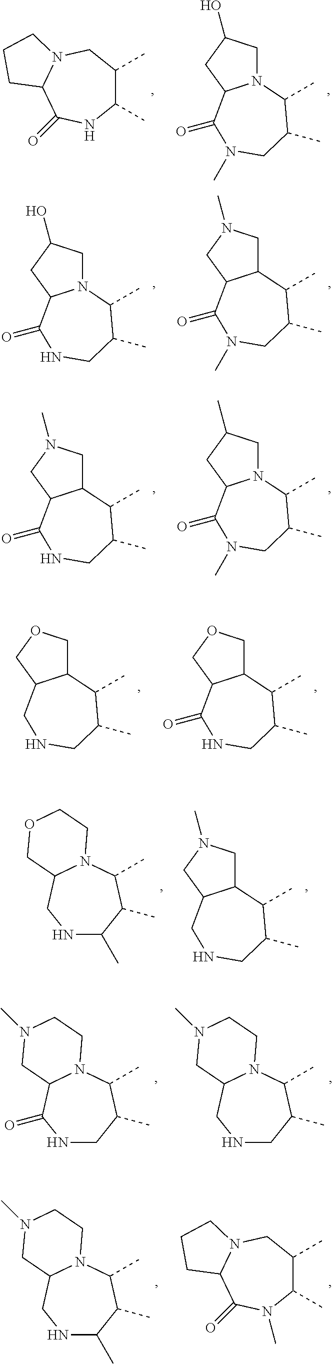

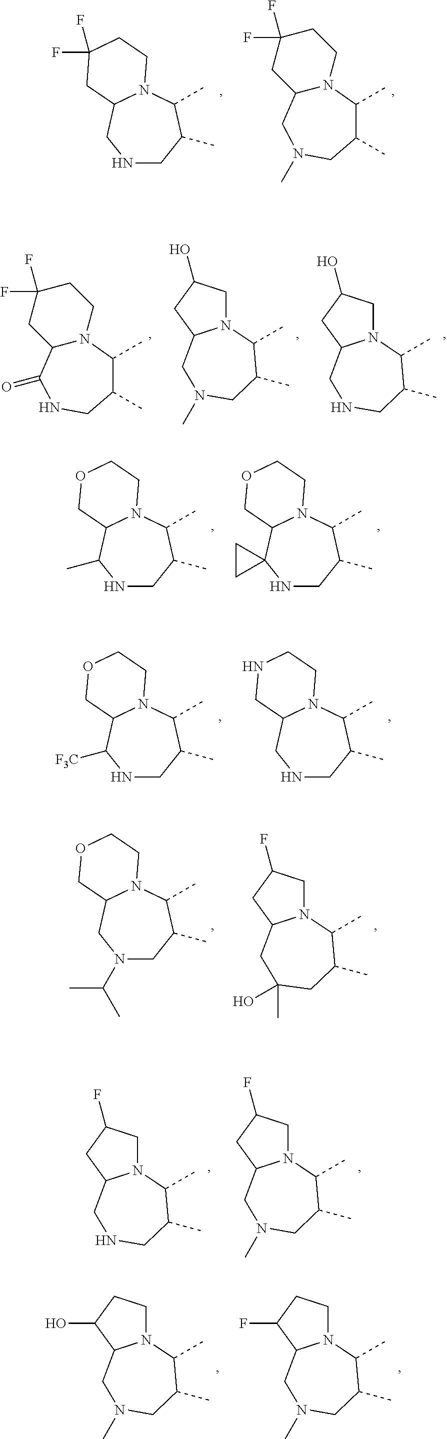

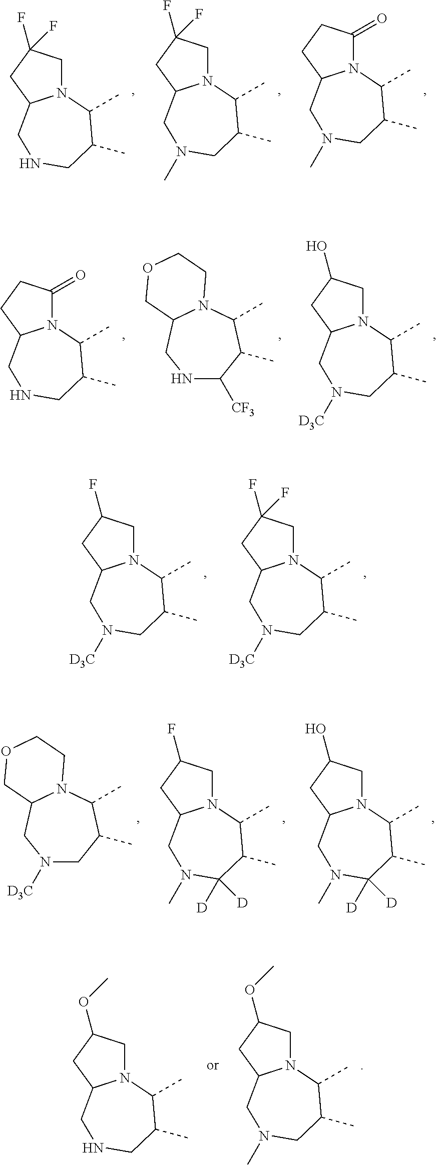

14. The compound according to claim 1, a stereoisomer or a pharmaceutically acceptable salt thereof, which is selected from: 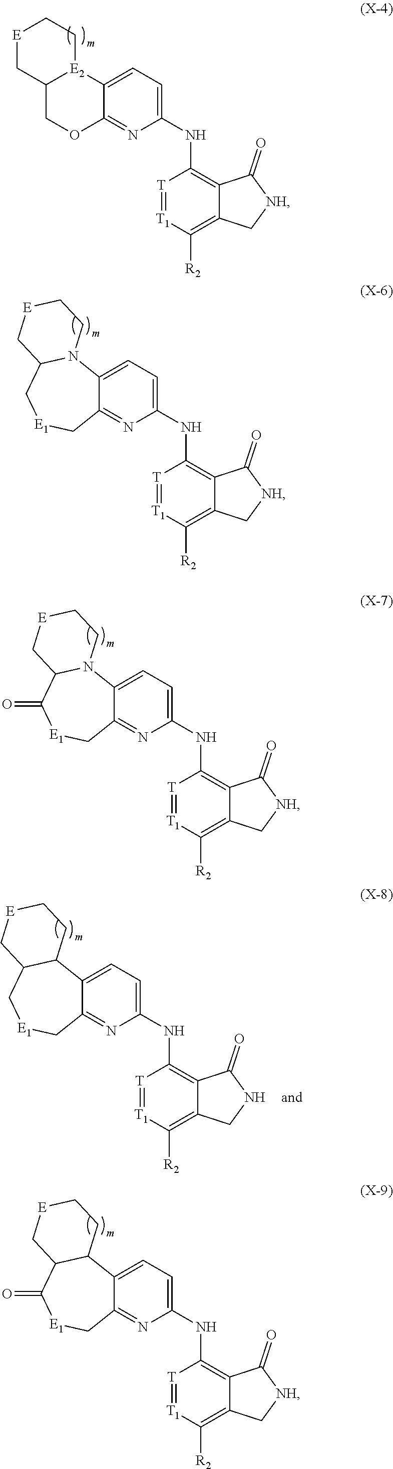
wherein, E, E₁, T, T₁, R₂ and m are as defined in claim 1,
E₂ is selected from N and CH.

15. The compound according to claim 1, a stereoisomer or a pharmaceutically acceptable salt thereof, which is selected from: 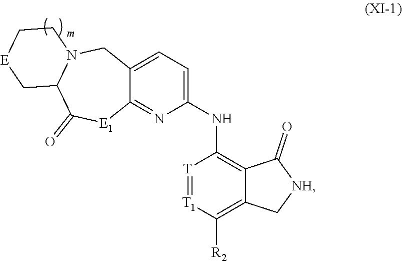
wherein E, E₁, T, T₁, R₂ and m are as defined in claim 1.

16. The following compound, a stereoisomer or a pharmaceutically acceptable salt thereof,

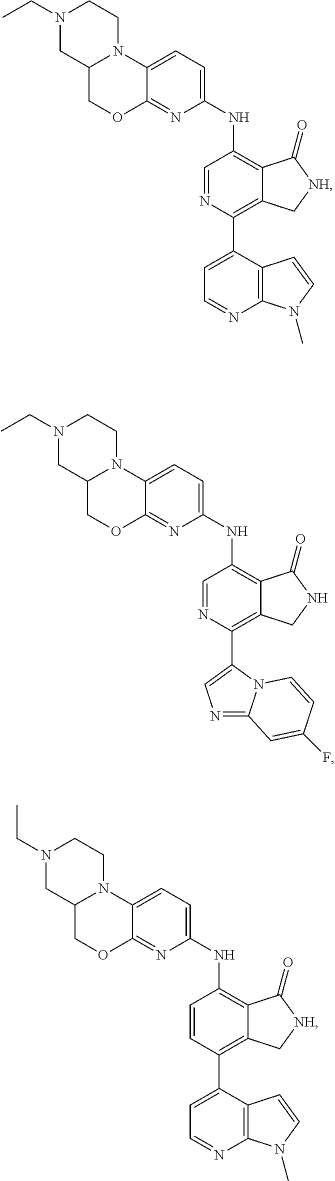 [SMILES: CCN1CCN2c3ccc(Nc4cnc(-c5ccnc6c5ccn6C)c5c4C(=O)NC5)nc3OCC2C1 ; CCN1CCN2c3ccc(Nc4cnc(-c5cnc6cc(F)ccn56)c5c4C(=O)NC5)nc3OCC2C1 ; CCN1CCN2c3ccc(Nc4ccc(-c5ccnc6c5ccn6C)c5c4C(=O)NC5)nc3OCC2C1]

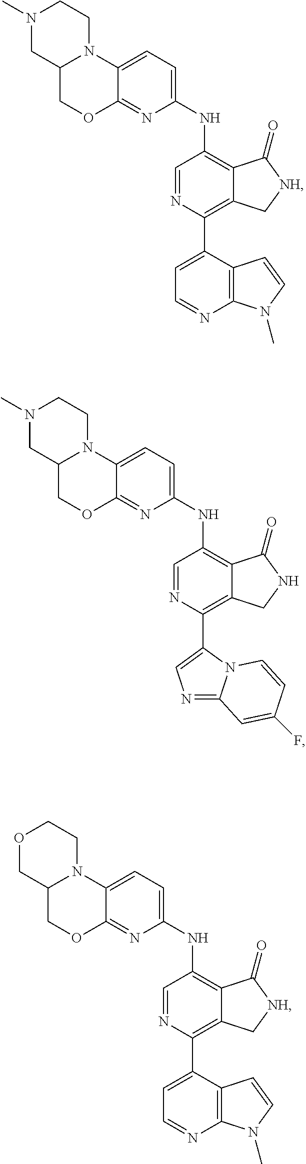 [SMILES: CN1CCN2c3ccc(Nc4cnc(-c5ccnc6c5ccn6C)c5c4C(=O)NC5)nc3OCC2C1 ; CN1CCN2c3ccc(Nc4cnc(-c5cnc6cc(F)ccn56)c5c4C(=O)NC5)nc3OCC2C1 ; Cn1ccc2c(-c3ncc(Nc4ccc5c(n4)OCC4COCCN54)c4c3CNC4=O)ccnc21]

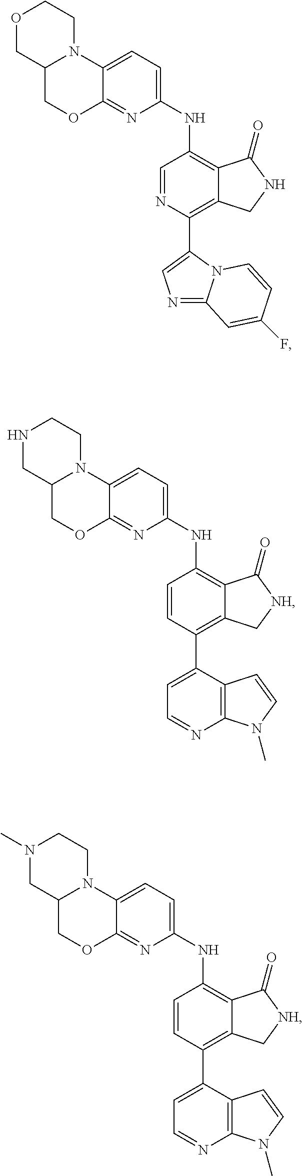 [SMILES: O=C1NCc2c(-c3cnc4cc(F)ccn34)ncc(Nc3ccc4c(n3)OCC3COCCN43)c21 ; Cn1ccc2c(-c3ccc(Nc4ccc5c(n4)OCC4CNCCN54)c4c3CNC4=O)ccnc21 ; CN1CCN2c3ccc(Nc4ccc(-c5ccnc6c5ccn6C)c5c4C(=O)NC5)nc3OCC2C1]

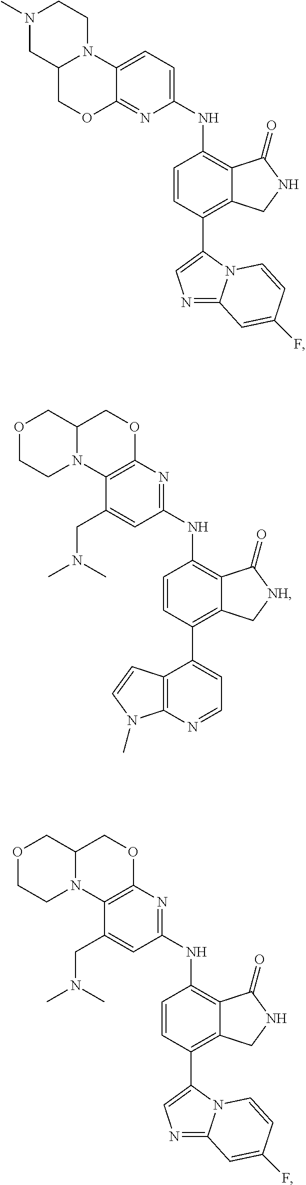 [SMILES: CN1CCN2c3ccc(Nc4ccc(-c5cnc6cc(F)ccn56)c5c4C(=O)NC5)nc3OCC2C1 ; CN(C)Cc1cc(Nc2ccc(-c3ccnc4c3ccn4C)c3c2C(=O)NC3)nc2c1N1CCOCC1CO2 ; CN(C)Cc1cc(Nc2ccc(-c3cnc4cc(F)ccn34)c3c2C(=O)NC3)nc2c1N1CCOCC1CO2]

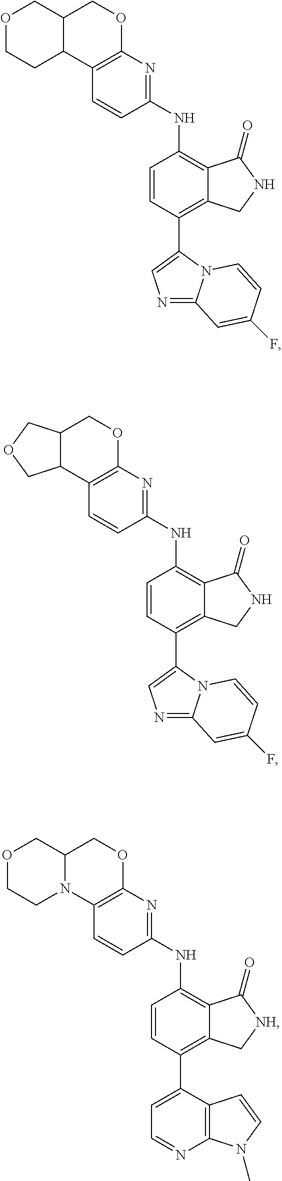 [SMILES: O=C1NCc2c(-c3cnc4cc(F)ccn34)ccc(Nc3ccc4c(n3)OCC3COCCC43)c21 ; O=C1NCc2c(-c3cnc4cc(F)ccn34)ccc(Nc3ccc4c(n3)OCC3COCC43)c21 ; Cn1ccc2c(-c3ccc(Nc4ccc5c(n4)OCC4COCCN54)c4c3CNC4=O)ccnc21]

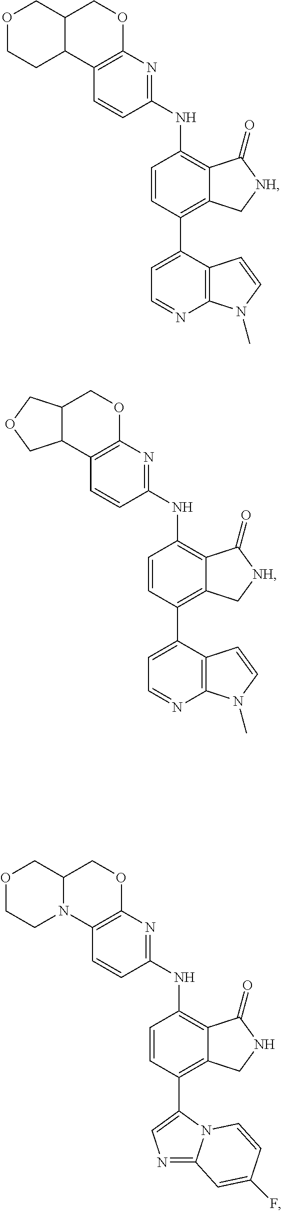 [SMILES: Cn1ccc2c(-c3ccc(Nc4ccc5c(n4)OCC4COCCC54)c4c3CNC4=O)ccnc21 ; Cn1ccc2c(-c3ccc(Nc4ccc5c(n4)OCC4COCC54)c4c3CNC4=O)ccnc21 ; O=C1NCc2c(-c3cnc4cc(F)ccn34)ccc(Nc3ccc4c(n3)OCC3COCCN43)c21]

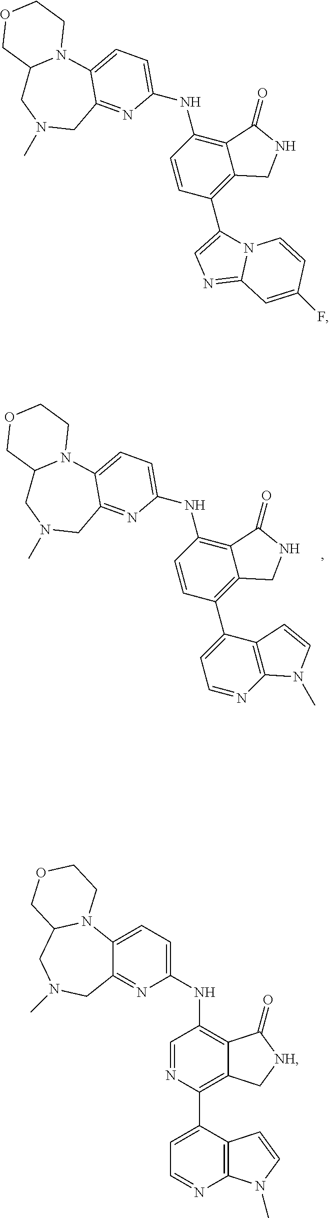

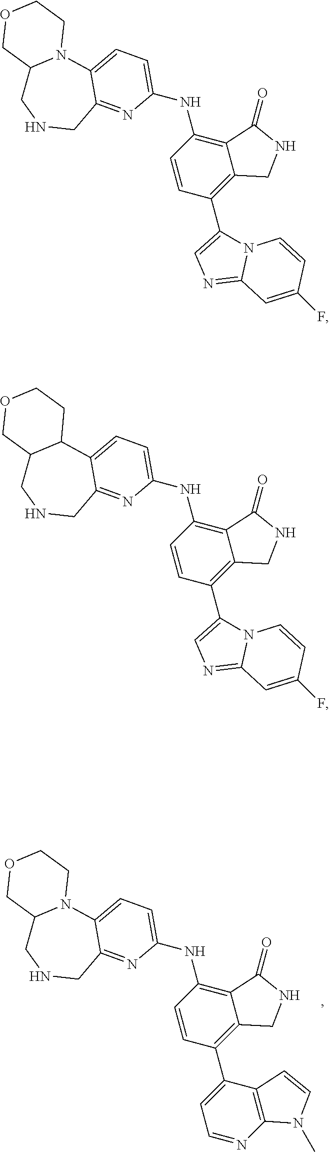 [SMILES: O=C1NCc2c(-c3cnc4cc(F)ccn34)ccc(Nc3ccc4c(n3)CNCC3COCCN43)c21 ; O=C1NCc2c(-c3cnc4cc(F)ccn34)ccc(Nc3ccc4c(n3)CNCC3COCCC43)c21 ; Cn1ccc2c(-c3ccc(Nc4ccc5c(n4)CNCC4COCCN54)c4c3CNC4=O)ccnc21]

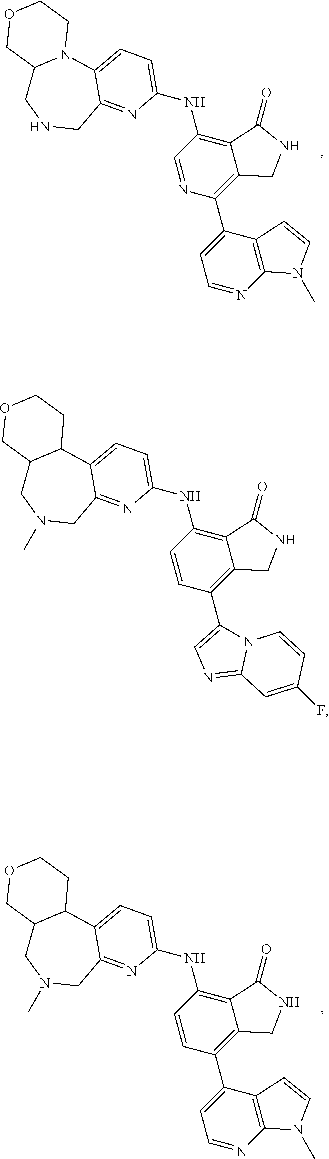 [SMILES: Cn1ccc2c(-c3ncc(Nc4ccc5c(n4)CNCC4COCCN54)c4c3CNC4=O)ccnc21 ; CN1Cc2nc(Nc3ccc(-c4cnc5cc(F)ccn45)c4c3C(=O)NC4)ccc2C2CCOCC2C1 ; CN1Cc2nc(Nc3ccc(-c4ccnc5c4ccn5C)c4c3C(=O)NC4)ccc2C2CCOCC2C1]

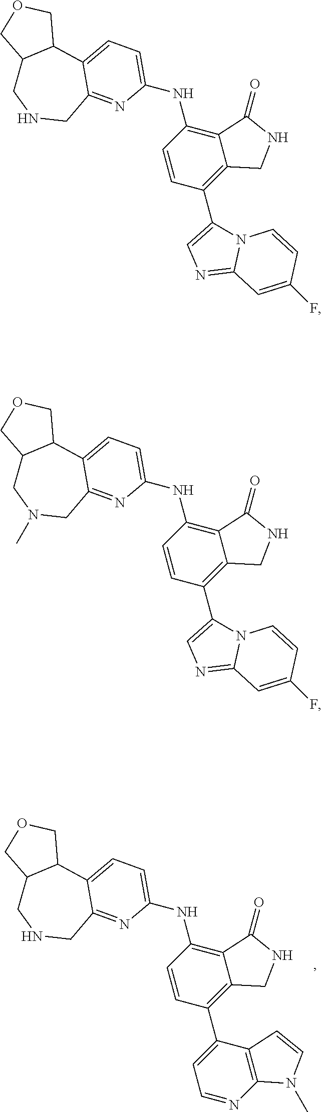 [SMILES: O=C1NCc2c(-c3cnc4cc(F)ccn34)ccc(Nc3ccc4c(n3)CNCC3COCC43)c21 ; CN1Cc2nc(Nc3ccc(-c4cnc5cc(F)ccn45)c4c3C(=O)NC4)ccc2C2COCC2C1 ; Cn1ccc2c(-c3ccc(Nc4ccc5c(n4)CNCC4COCC54)c4c3CNC4=O)ccnc21]

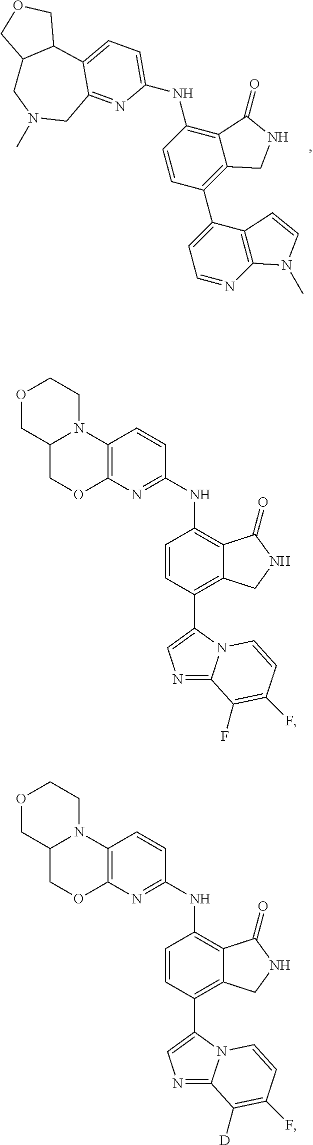 [SMILES: CN1Cc2nc(Nc3ccc(-c4ccnc5c4ccn5C)c4c3C(=O)NC4)ccc2C2COCC2C1 ; O=C1NCc2c(-c3cnc4c(F)c(F)ccn34)ccc(Nc3ccc4c(n3)OCC3COCCN43)c21 ; [2H]c1c(F)ccn2c(-c3ccc(Nc4ccc5c(n4)OCC4COCCN54)c4c3CNC4=O)cnc12]

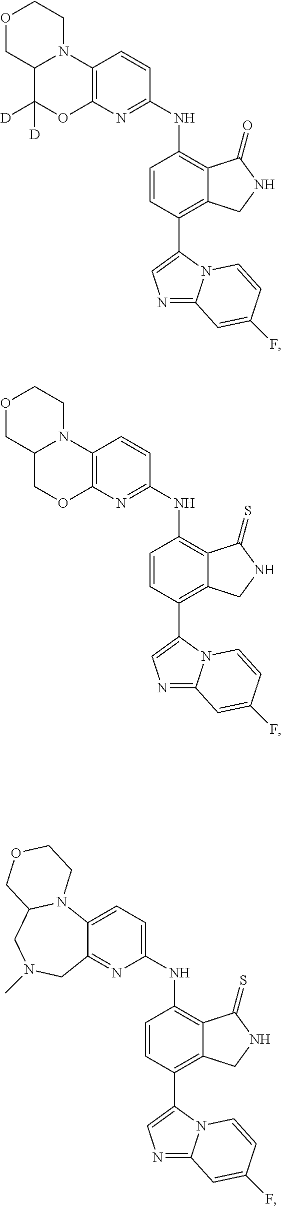 [SMILES: [2H]C1([2H])Oc2nc(Nc3ccc(-c4cnc5cc(F)ccn45)c4c3C(=O)NC4)ccc2N2CCOCC21 ; Fc1ccn2c(-c3ccc(Nc4ccc5c(n4)OCC4COCCN54)c4c3CNC4=S)cnc2c1 ; CN1Cc2nc(Nc3ccc(-c4cnc5cc(F)ccn45)c4c3C(=S)NC4)ccc2N2CCOCC2C1]

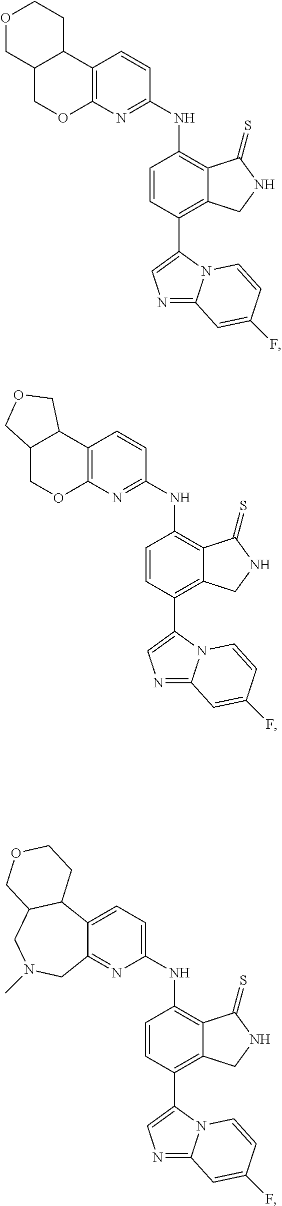 [SMILES: Fc1ccn2c(-c3ccc(Nc4ccc5c(n4)OCC4COCCC54)c4c3CNC4=S)cnc2c1 ; Fc1ccn2c(-c3ccc(Nc4ccc5c(n4)OCC4COCC54)c4c3CNC4=S)cnc2c1 ; CN1Cc2nc(Nc3ccc(-c4cnc5cc(F)ccn45)c4c3C(=S)NC4)ccc2C2CCOCC2C1]

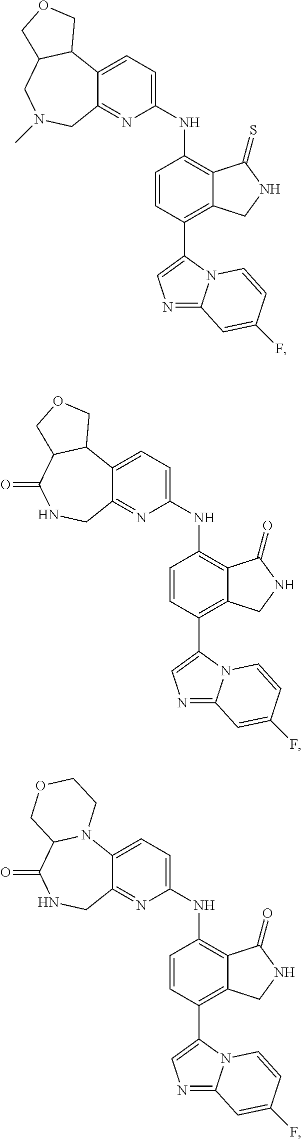 [SMILES: CN1Cc2nc(Nc3ccc(-c4cnc5cc(F)ccn45)c4c3C(=S)NC4)ccc2C2COCC2C1 ; O=C1NCc2c(-c3cnc4cc(F)ccn34)ccc(Nc3ccc4c(n3)CNC(=O)C3COCC43)c21 ; O=C1NCc2c(-c3cnc4cc(F)ccn34)ccc(Nc3ccc4c(n3)CNC(=O)C3COCCN43)c21]

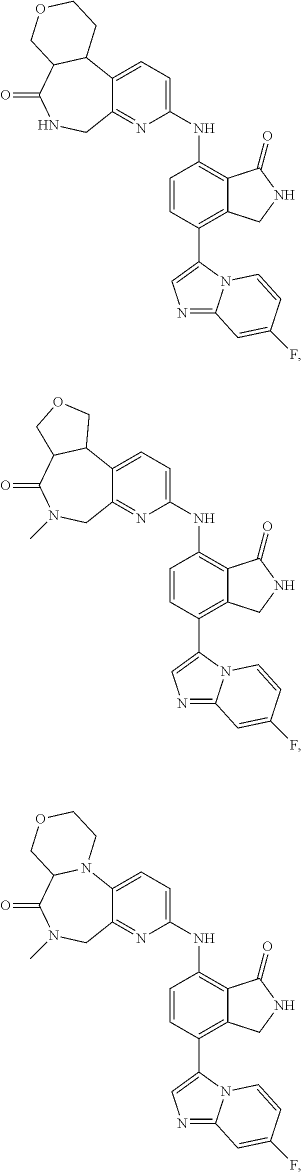 [SMILES: O=C1NCc2c(-c3cnc4cc(F)ccn34)ccc(Nc3ccc4c(n3)CNC(=O)C3COCCC43)c21 ; CN1Cc2nc(Nc3ccc(-c4cnc5cc(F)ccn45)c4c3C(=O)NC4)ccc2C2COCC2C1=O ; CN1Cc2nc(Nc3ccc(-c4cnc5cc(F)ccn45)c4c3C(=O)NC4)ccc2N2CCOCC2C1=O]

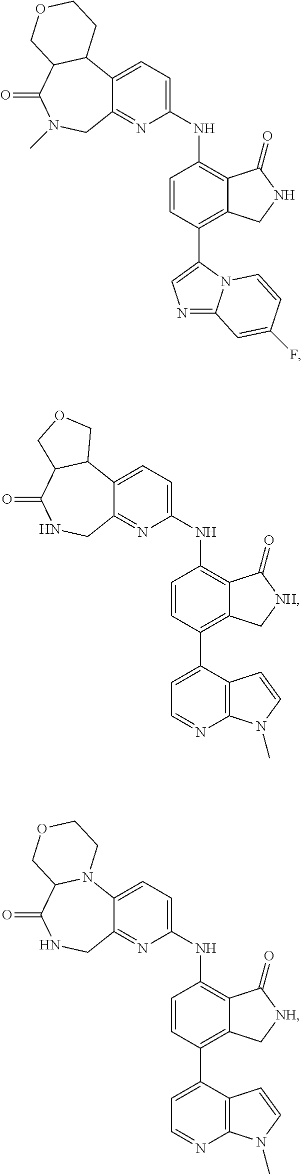 [SMILES: CN1Cc2nc(Nc3ccc(-c4cnc5cc(F)ccn45)c4c3C(=O)NC4)ccc2C2CCOCC2C1=O ; Cn1ccc2c(-c3ccc(Nc4ccc5c(n4)CNC(=O)C4COCC54)c4c3CNC4=O)ccnc21 ; Cn1ccc2c(-c3ccc(Nc4ccc5c(n4)CNC(=O)C4COCCN54)c4c3CNC4=O)ccnc21]

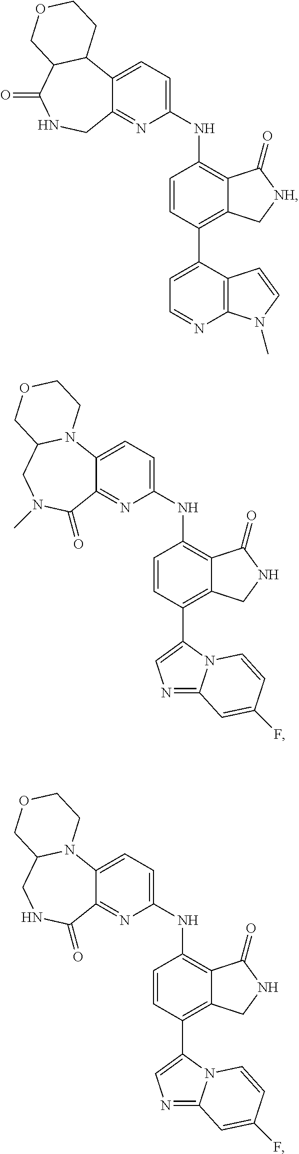 [SMILES: Cn1ccc2c(-c3ccc(Nc4ccc5c(n4)CNC(=O)C4COCCC54)c4c3CNC4=O)ccnc21 ; CN1CC2COCCN2c2ccc(Nc3ccc(-c4cnc5cc(F)ccn45)c4c3C(=O)NC4)nc2C1=O ; O=C1NCC2COCCN2c2ccc(Nc3ccc(-c4cnc5cc(F)ccn45)c4c3C(=O)NC4)nc21]

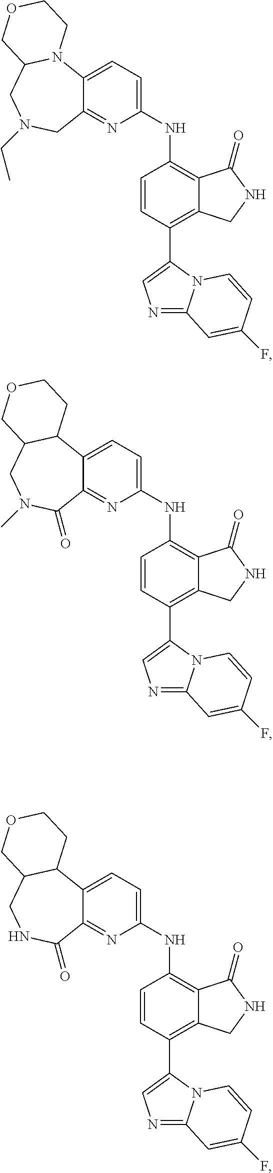 [SMILES: CCN1Cc2nc(Nc3ccc(-c4cnc5cc(F)ccn45)c4c3C(=O)NC4)ccc2N2CCOCC2C1 ; CN1CC2COCCC2c2ccc(Nc3ccc(-c4cnc5cc(F)ccn45)c4c3C(=O)NC4)nc2C1=O ; O=C1NCC2COCCC2c2ccc(Nc3ccc(-c4cnc5cc(F)ccn45)c4c3C(=O)NC4)nc21]

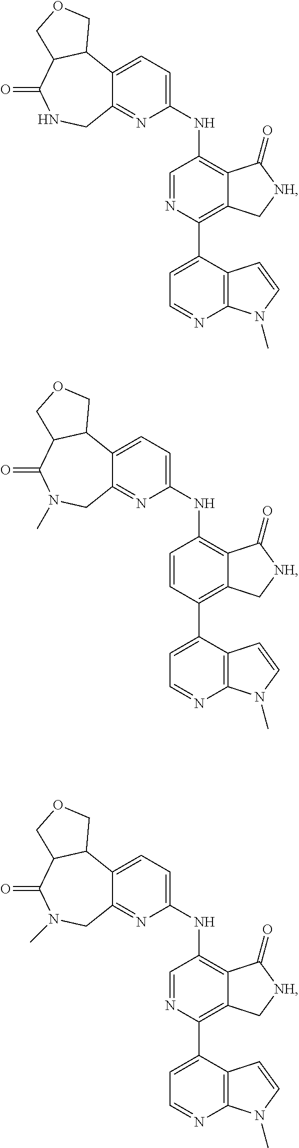 [SMILES: Cn1ccc2c(-c3ncc(Nc4ccc5c(n4)CNC(=O)C4COCC54)c4c3CNC4=O)ccnc21 ; CN1Cc2nc(Nc3ccc(-c4ccnc5c4ccn5C)c4c3C(=O)NC4)ccc2C2COCC2C1=O ; CN1Cc2nc(Nc3cnc(-c4ccnc5c4ccn5C)c4c3C(=O)NC4)ccc2C2COCC2C1=O]

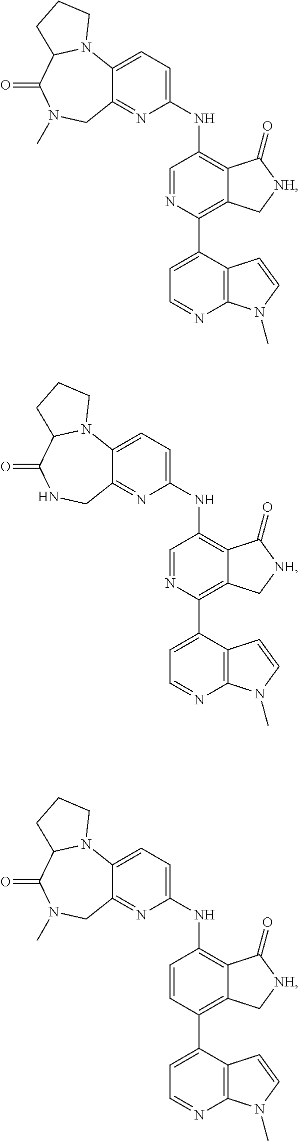 [SMILES: CN1Cc2nc(Nc3cnc(-c4ccnc5c4ccn5C)c4c3C(=O)NC4)ccc2N2CCCC2C1=O ; Cn1ccc2c(-c3ncc(Nc4ccc5c(n4)CNC(=O)C4CCCN54)c4c3CNC4=O)ccnc21 ; CN1Cc2nc(Nc3ccc(-c4ccnc5c4ccn5C)c4c3C(=O)NC4)ccc2N2CCCC2C1=O]

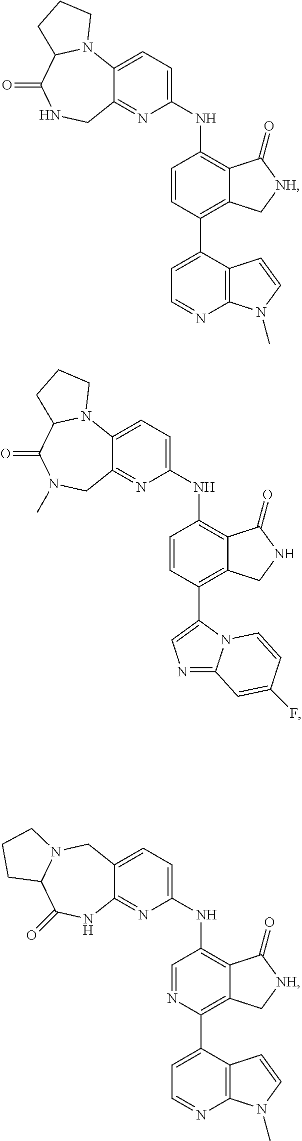 [SMILES: Cn1ccc2c(-c3ccc(Nc4ccc5c(n4)CNC(=O)C4CCCN54)c4c3CNC4=O)ccnc21 ; CN1Cc2nc(Nc3ccc(-c4cnc5cc(F)ccn45)c4c3C(=O)NC4)ccc2N2CCCC2C1=O ; Cn1ccc2c(-c3ncc(Nc4ccc5c(n4)NC(=O)C4CCCN4C5)c4c3CNC4=O)ccnc21]

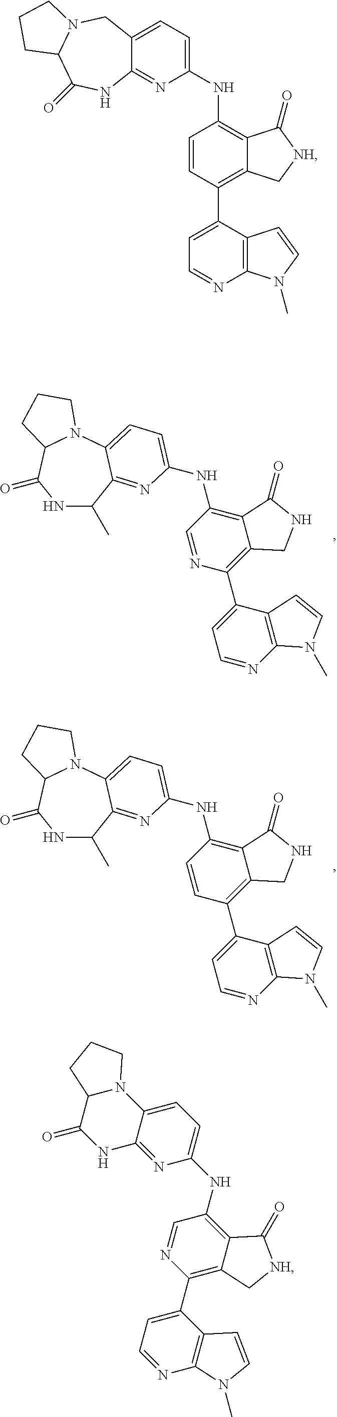 [SMILES: Cn1ccc2c(-c3ccc(Nc4ccc5c(n4)NC(=O)C4CCCN4C5)c4c3CNC4=O)ccnc21 ; CC1NC(=O)C2CCCN2c2ccc(Nc3cnc(-c4ccnc5c4ccn5C)c4c3C(=O)NC4)nc21 ; CC1NC(=O)C2CCCN2c2ccc(Nc3ccc(-c4ccnc5c4ccn5C)c4c3C(=O)NC4)nc21 ; Cn1ccc2c(-c3ncc(Nc4ccc5c(n4)NC(=O)C4CCCN54)c4c3CNC4=O)ccnc21]

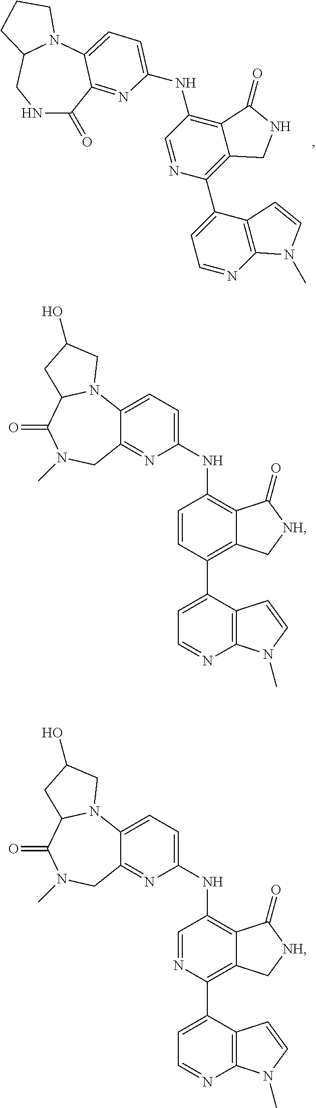 [SMILES: Cn1ccc2c(-c3ncc(Nc4ccc5c(n4)C(=O)NCC4CCCN54)c4c3CNC4=O)ccnc21 ; CN1Cc2nc(Nc3ccc(-c4ccnc5c4ccn5C)c4c3C(=O)NC4)ccc2N2CC(O)CC2C1=O ; CN1Cc2nc(Nc3cnc(-c4ccnc5c4ccn5C)c4c3C(=O)NC4)ccc2N2CC(O)CC2C1=O]

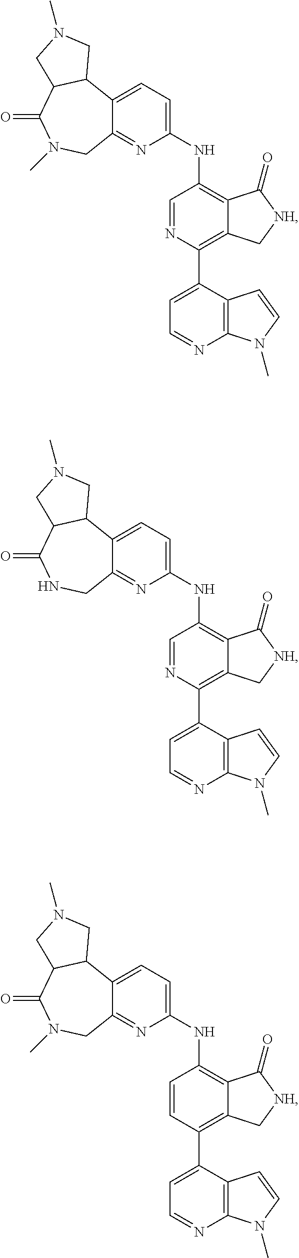 [SMILES: CN1CC2C(=O)N(C)Cc3nc(Nc4cnc(-c5ccnc6c5ccn6C)c5c4C(=O)NC5)ccc3C2C1 ; CN1CC2C(=O)NCc3nc(Nc4cnc(-c5ccnc6c5ccn6C)c5c4C(=O)NC5)ccc3C2C1 ; CN1CC2C(=O)N(C)Cc3nc(Nc4ccc(-c5ccnc6c5ccn6C)c5c4C(=O)NC5)ccc3C2C1]

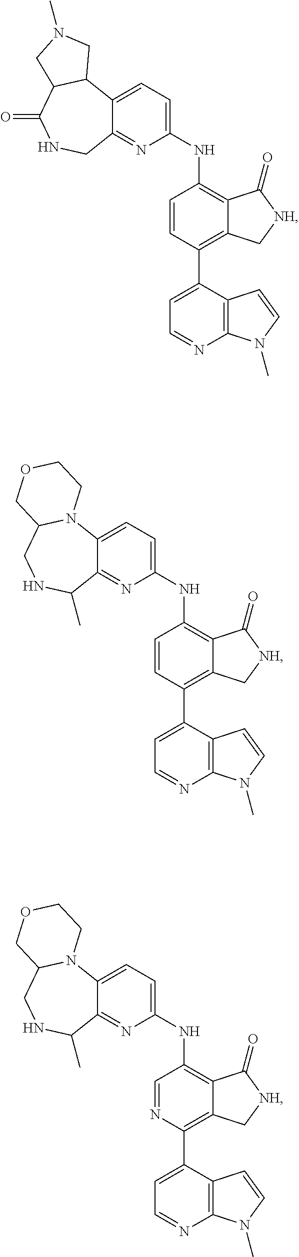 [SMILES: CN1CC2C(=O)NCc3nc(Nc4ccc(-c5ccnc6c5ccn6C)c5c4C(=O)NC5)ccc3C2C1 ; CC1NCC2COCCN2c2ccc(Nc3ccc(-c4ccnc5c4ccn5C)c4c3C(=O)NC4)nc21 ; CC1NCC2COCCN2c2ccc(Nc3cnc(-c4ccnc5c4ccn5C)c4c3C(=O)NC4)nc21]

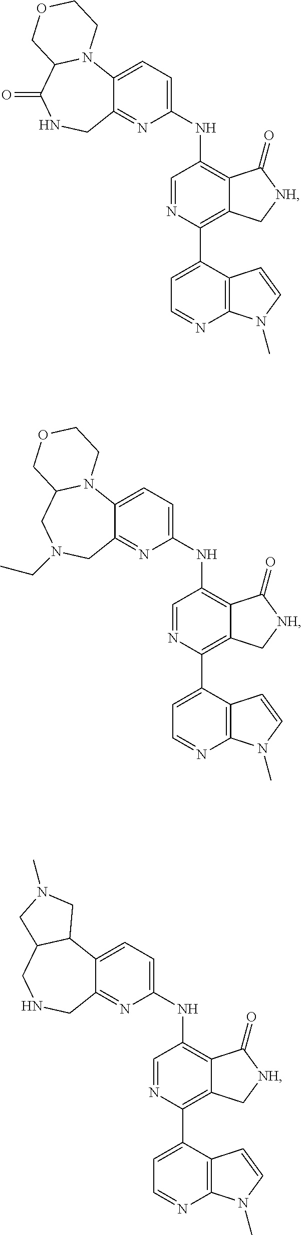 [SMILES: Cn1ccc2c(-c3ncc(Nc4ccc5c(n4)CNC(=O)C4COCCN54)c4c3CNC4=O)ccnc21 ; CCN1Cc2nc(Nc3cnc(-c4ccnc5c4ccn5C)c4c3C(=O)NC4)ccc2N2CCOCC2C1 ; CN1CC2CNCc3nc(Nc4cnc(-c5ccnc6c5ccn6C)c5c4C(=O)NC5)ccc3C2C1]

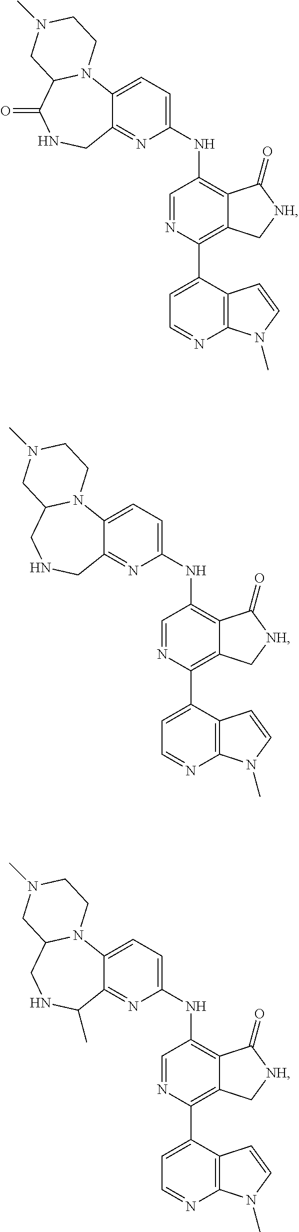 [SMILES: CN1CCN2c3ccc(Nc4cnc(-c5ccnc6c5ccn6C)c5c4C(=O)NC5)nc3CNC(=O)C2C1 ; CN1CCN2c3ccc(Nc4cnc(-c5ccnc6c5ccn6C)c5c4C(=O)NC5)nc3CNCC2C1 ; CC1NCC2CN(C)CCN2c2ccc(Nc3cnc(-c4ccnc5c4ccn5C)c4c3C(=O)NC4)nc21]

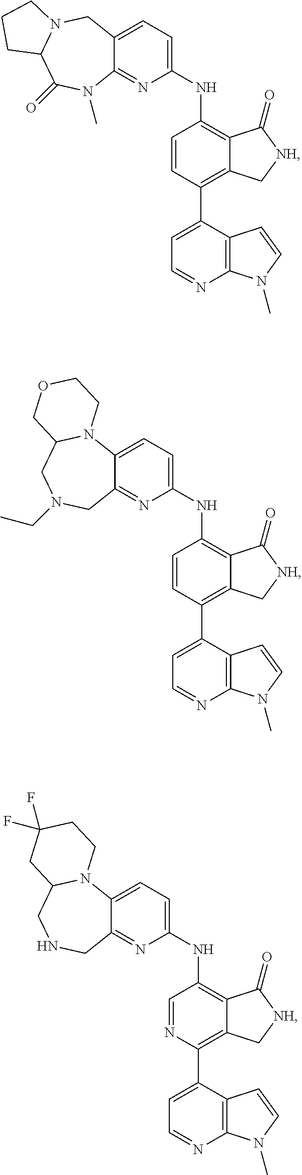 [SMILES: CN1C(=O)C2CCCN2Cc2ccc(Nc3ccc(-c4ccnc5c4ccn5C)c4c3C(=O)NC4)nc21 ; CCN1Cc2nc(Nc3ccc(-c4ccnc5c4ccn5C)c4c3C(=O)NC4)ccc2N2CCOCC2C1 ; Cn1ccc2c(-c3ncc(Nc4ccc5c(n4)CNCC4CC(F)(F)CCN54)c4c3CNC4=O)ccnc21]

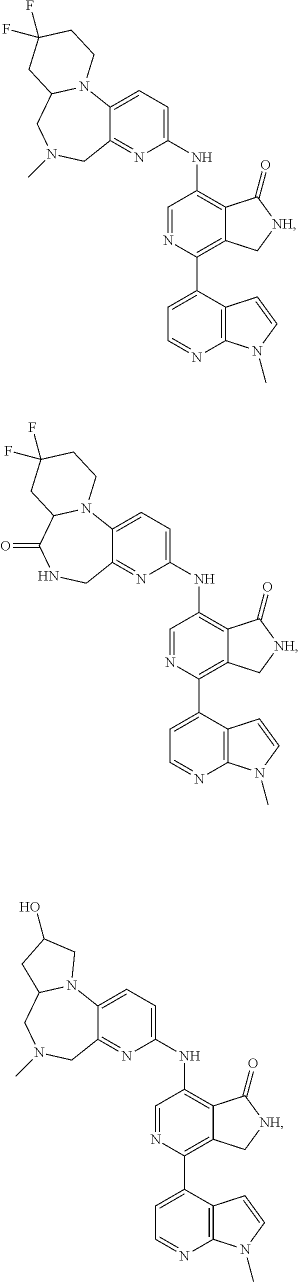 [SMILES: CN1Cc2nc(Nc3cnc(-c4ccnc5c4ccn5C)c4c3C(=O)NC4)ccc2N2CCC(F)(F)CC2C1 ; Cn1ccc2c(-c3ncc(Nc4ccc5c(n4)CNC(=O)C4CC(F)(F)CCN54)c4c3CNC4=O)ccnc21 ; CN1Cc2nc(Nc3cnc(-c4ccnc5c4ccn5C)c4c3C(=O)NC4)ccc2N2CC(O)CC2C1]

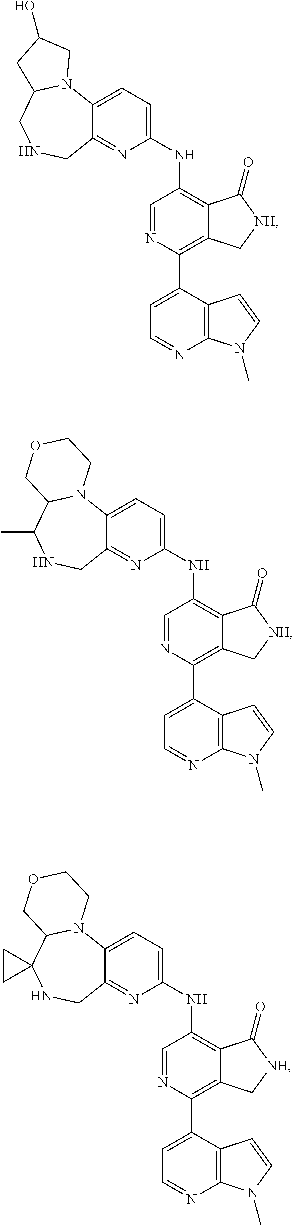 [SMILES: Cn1ccc2c(-c3ncc(Nc4ccc5c(n4)CNCC4CC(O)CN54)c4c3CNC4=O)ccnc21 ; CC1NCc2nc(Nc3cnc(-c4ccnc5c4ccn5C)c4c3C(=O)NC4)ccc2N2CCOCC12 ; Cn1ccc2c(-c3ncc(Nc4ccc5c(n4)CNC4(CC4)C4COCCN54)c4c3CNC4=O)ccnc21]

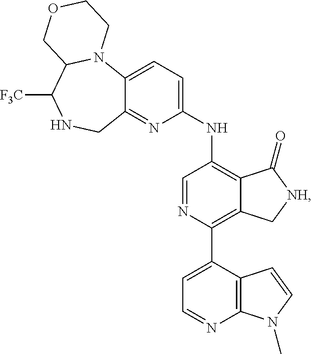 [SMILES: Cn1ccc2c(-c3ncc(Nc4ccc5c(n4)CNC(C(F)(F)F)C4COCCN54)c4c3CNC4=O)ccnc21]

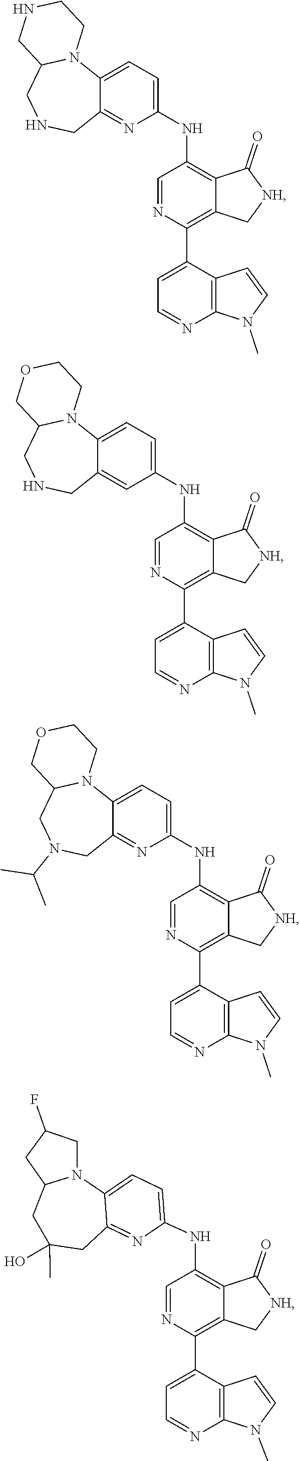 [SMILES: Cn1ccc2c(-c3ncc(Nc4ccc5c(n4)CNCC4CNCCN54)c4c3CNC4=O)ccnc21 ; Cn1ccc2c(-c3ncc(Nc4ccc5c(c4)CNCC4COCCN54)c4c3CNC4=O)ccnc21 ; CC(C)N1Cc2nc(Nc3cnc(-c4ccnc5c4ccn5C)c4c3C(=O)NC4)ccc2N2CCOCC2C1 ; Cn1ccc2c(-c3ncc(Nc4ccc5c(n4)CC(C)(O)CC4CC(F)CN54)c4c3CNC4=O)ccnc21]

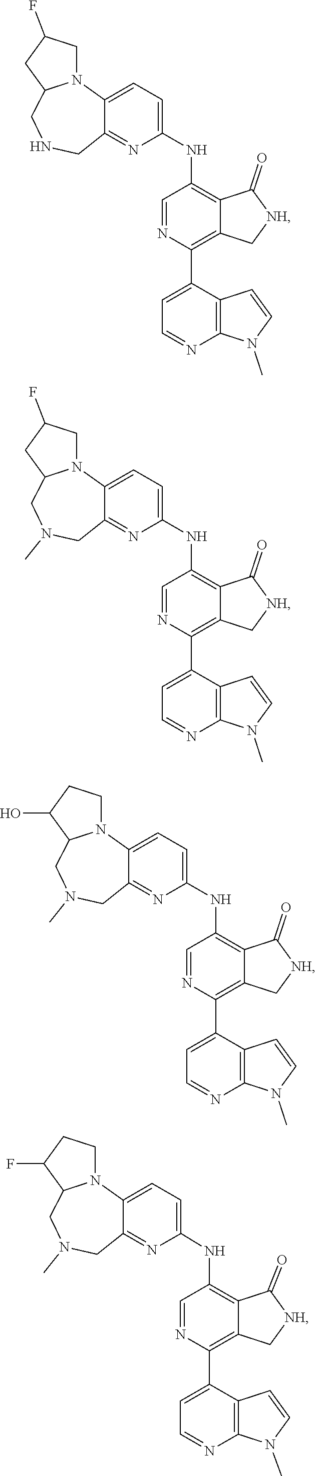 [SMILES: Cn1ccc2c(-c3ncc(Nc4ccc5c(n4)CNCC4CC(F)CN54)c4c3CNC4=O)ccnc21 ; CN1Cc2nc(Nc3cnc(-c4ccnc5c4ccn5C)c4c3C(=O)NC4)ccc2N2CC(F)CC2C1 ; CN1Cc2nc(Nc3cnc(-c4ccnc5c4ccn5C)c4c3C(=O)NC4)ccc2N2CCC(O)C2C1 ; CN1Cc2nc(Nc3cnc(-c4ccnc5c4ccn5C)c4c3C(=O)NC4)ccc2N2CCC(F)C2C1]

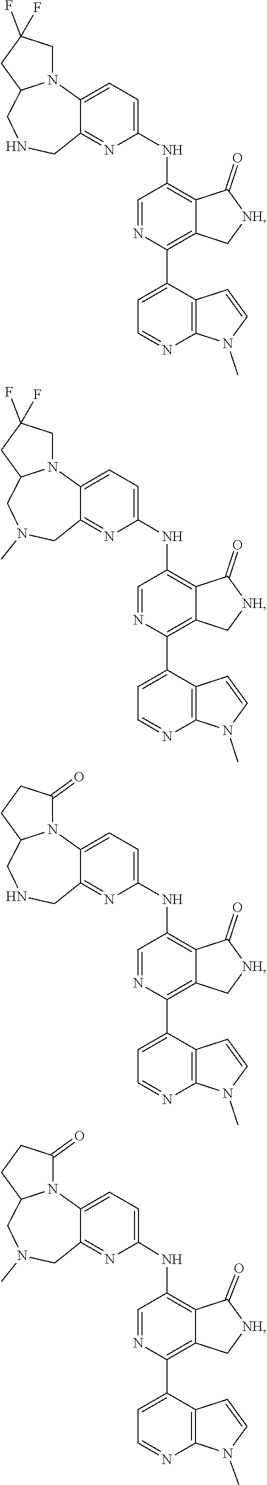 [SMILES: Cn1ccc2c(-c3ncc(Nc4ccc5c(n4)CNCC4CC(F)(F)CN54)c4c3CNC4=O)ccnc21 ; CN1Cc2nc(Nc3cnc(-c4ccnc5c4ccn5C)c4c3C(=O)NC4)ccc2N2CC(F)(F)CC2C1 ; Cn1ccc2c(-c3ncc(Nc4ccc5c(n4)CNCC4CCC(=O)N54)c4c3CNC4=O)ccnc21 ; CN1Cc2nc(Nc3cnc(-c4ccnc5c4ccn5C)c4c3C(=O)NC4)ccc2N2C(=O)CCC2C1]

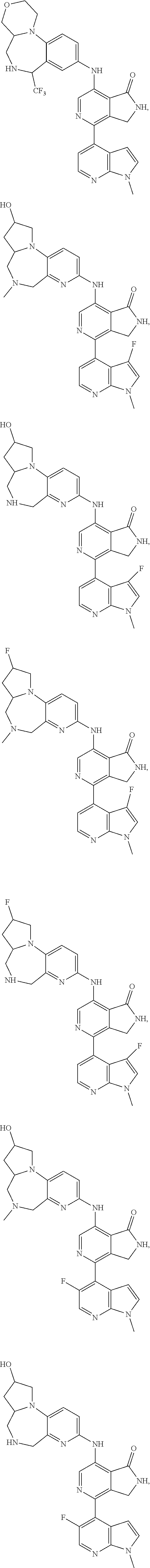

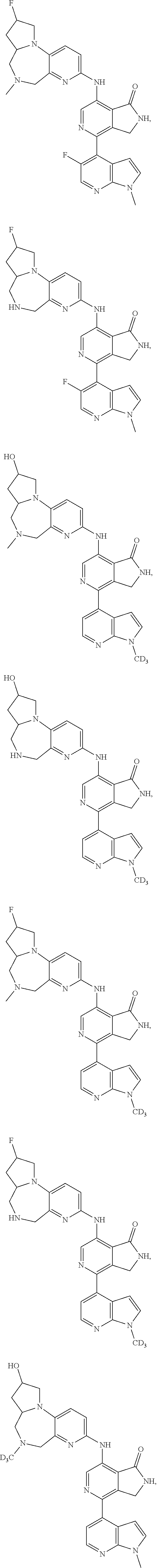

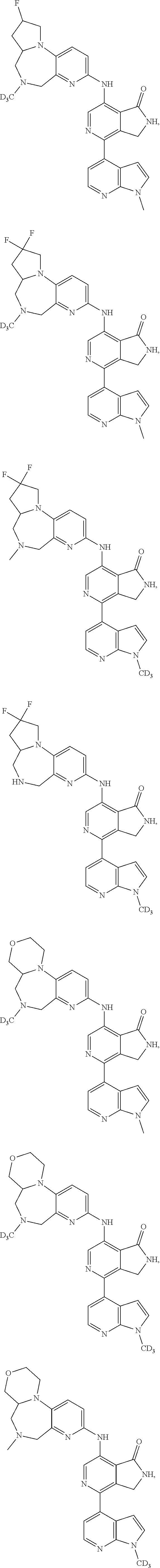

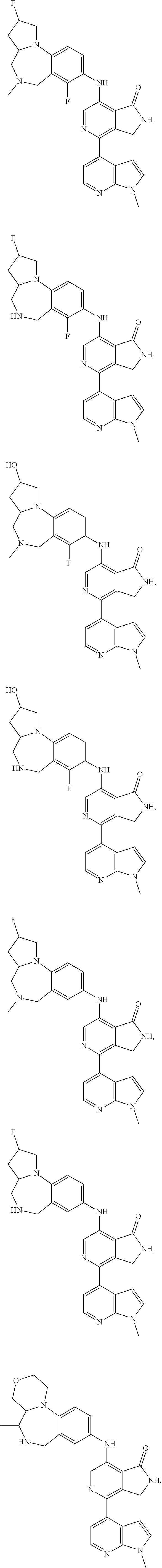

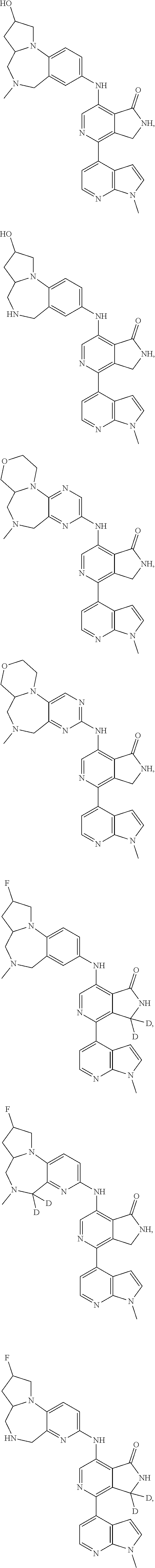

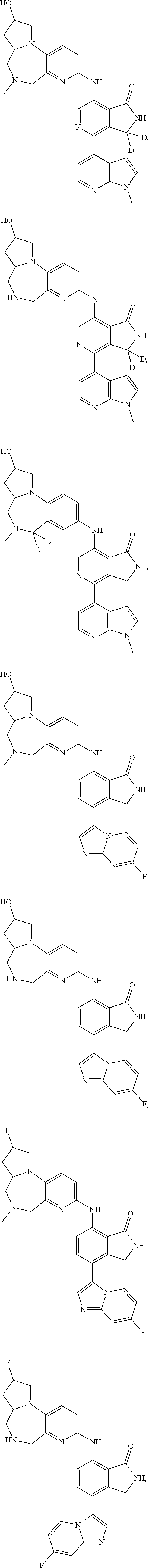

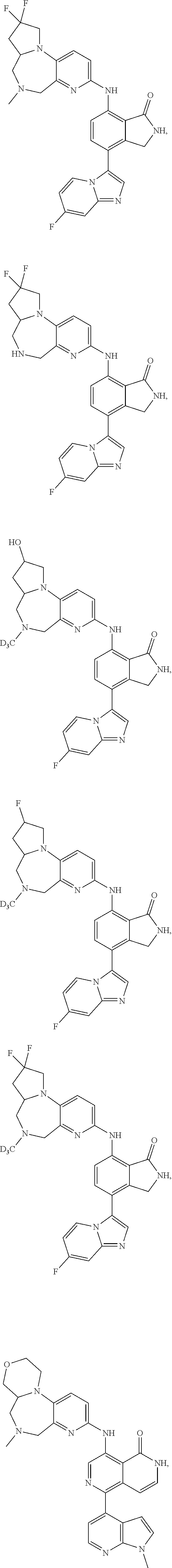 [SMILES: CN1Cc2nc(Nc3ccc(-c4cnc5cc(F)ccn45)c4c3C(=O)NC4)ccc2N2CC(F)(F)CC2C1 ; O=C1NCc2c(-c3cnc4cc(F)ccn34)ccc(Nc3ccc4c(n3)CNCC3CC(F)(F)CN43)c21 ; [2H]C([2H])([2H])N1Cc2nc(Nc3ccc(-c4cnc5cc(F)ccn45)c4c3C(=O)NC4)ccc2N2CC(O)CC2C1 ; [2H]C([2H])([2H])N1Cc2nc(Nc3ccc(-c4cnc5cc(F)ccn45)c4c3C(=O)NC4)ccc2N2CC(F)CC2C1 ; [2H]C([2H])([2H])N1Cc2nc(Nc3ccc(-c4cnc5cc(F)ccn45)c4c3C(=O)NC4)ccc2N2CC(F)(F)CC2C1 ; CN1Cc2nc(Nc3cnc(-c4ccnc5c4ccn5C)c4cc[nH]c(=O)c34)ccc2N2CCOCC2C1]

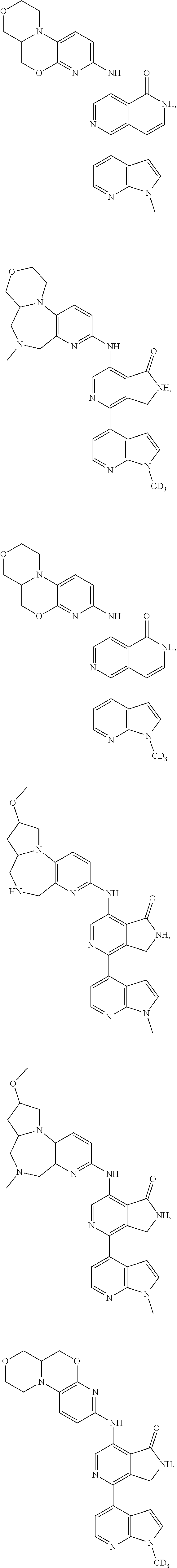 [SMILES: Cn1ccc2c(-c3ncc(Nc4ccc5c(n4)OCC4COCCN54)c4c(=O)[nH]ccc34)ccnc21 ; [2H]C([2H])([2H])n1ccc2c(-c3ncc(Nc4ccc5c(n4)CN(C)CC4COCCN54)c4c3CNC4=O)ccnc21 ; [2H]C([2H])([2H])n1ccc2c(-c3ncc(Nc4ccc5c(n4)OCC4COCCN54)c4c(=O)[nH]ccc34)ccnc21 ; COC1CC2CNCc3nc(Nc4cnc(-c5ccnc6c5ccn6C)c5c4C(=O)NC5)ccc3N2C1 ; COC1CC2CN(C)Cc3nc(Nc4cnc(-c5ccnc6c5ccn6C)c5c4C(=O)NC5)ccc3N2C1 ; [2H]C([2H])([2H])n1ccc2c(-c3ncc(Nc4ccc5c(n4)OCC4COCCN54)c4c3CNC4=O)ccnc21]

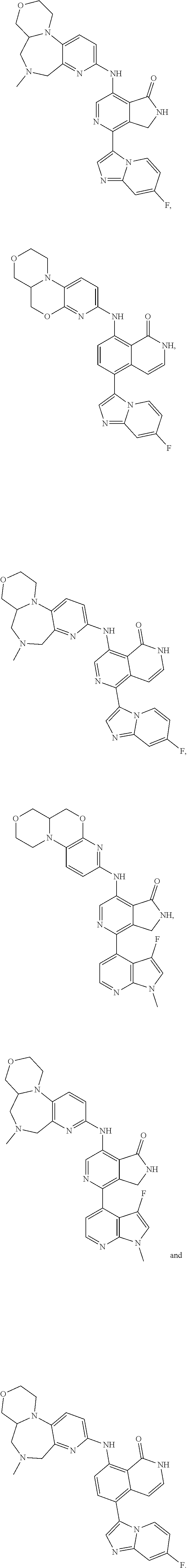 [SMILES: CN1Cc2nc(Nc3cnc(-c4cnc5cc(F)ccn45)c4c3C(=O)NC4)ccc2N2CCOCC2C1 ; O=c1[nH]ccc2c(-c3cnc4cc(F)ccn34)ccc(Nc3ccc4c(n3)OCC3COCCN43)c12 ; CN1Cc2nc(Nc3cnc(-c4cnc5cc(F)ccn45)c4cc[nH]c(=O)c34)ccc2N2CCOCC2C1 ; Cn1cc(F)c2c(-c3ncc(Nc4ccc5c(n4)OCC4COCCN54)c4c3CNC4=O)ccnc21 ; CN1Cc2nc(Nc3cnc(-c4ccnc5c4c(F)cn5C)c4c3C(=O)NC4)ccc2N2CCOCC2C1 ; CN1Cc2nc(Nc3ccc(-c4cnc5cc(F)ccn45)c4cc[nH]c(=O)c34)ccc2N2CCOCC2C1]

17. The compound according to claim 16, a stereoisomer or a pharmaceutically acceptable salt thereof, which is selected from:

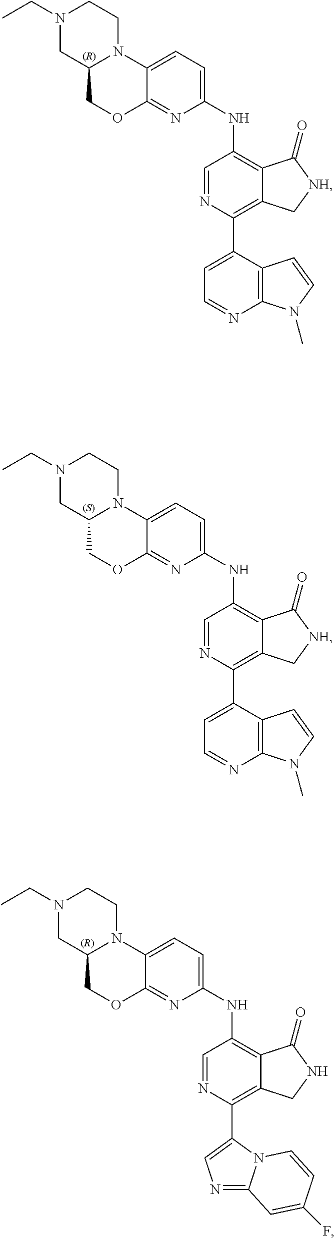

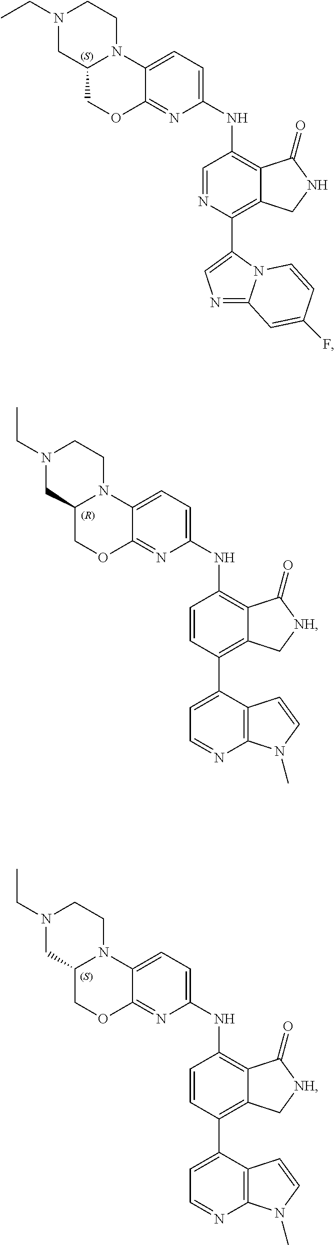

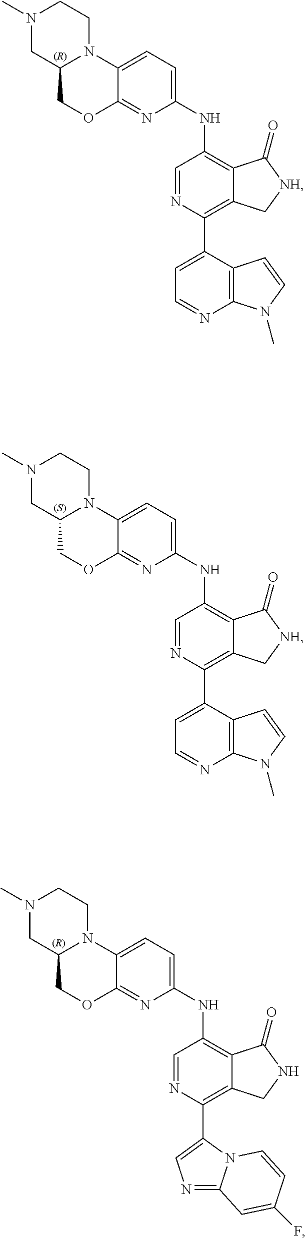

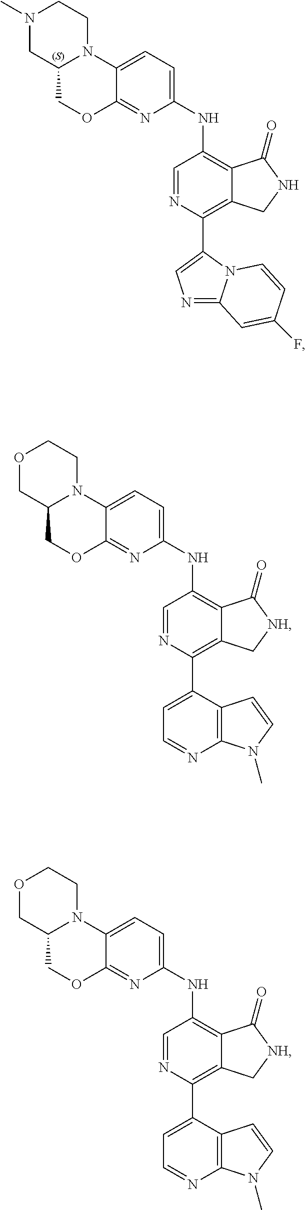

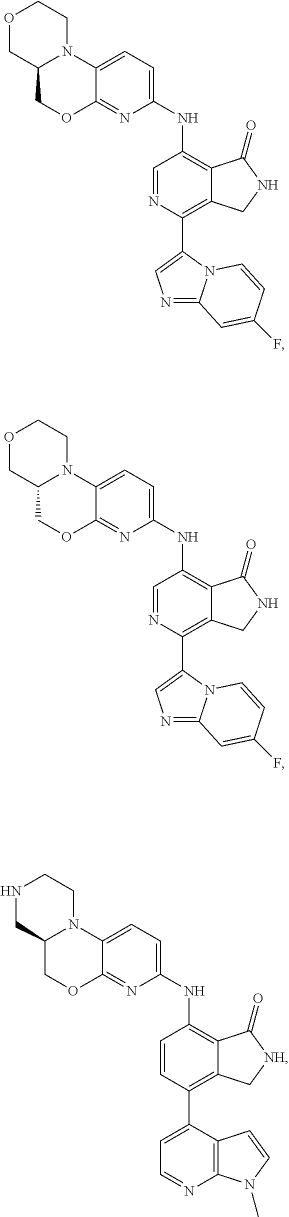 [SMILES: O=C1NCc2c(-c3cnc4cc(F)ccn34)ncc(Nc3ccc4c(n3)OC[C@H]3COCCN43)c21 ; O=C1NCc2c(-c3cnc4cc(F)ccn34)ncc(Nc3ccc4c(n3)OC[C@@H]3COCCN43)c21 ; Cn1ccc2c(-c3ccc(Nc4ccc5c(n4)OC[C@H]4CNCCN54)c4c3CNC4=O)ccnc21]

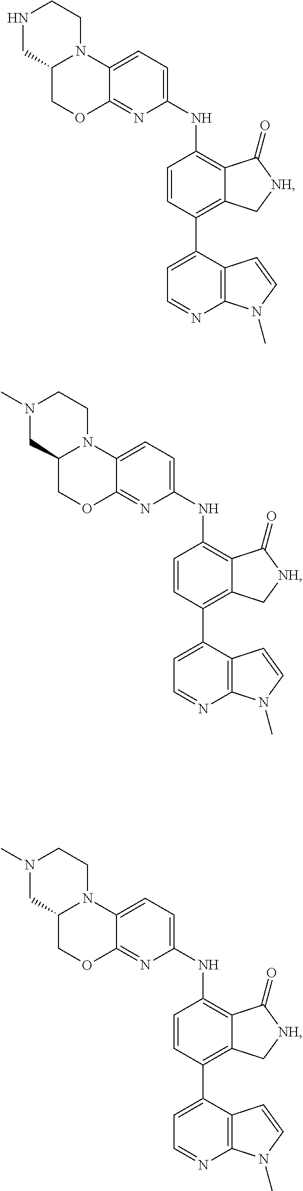 [SMILES: Cn1ccc2c(-c3ccc(Nc4ccc5c(n4)OC[C@@H]4CNCCN54)c4c3CNC4=O)ccnc21 ; CN1CCN2c3ccc(Nc4ccc(-c5ccnc6c5ccn6C)c5c4C(=O)NC5)nc3OC[C@H]2C1 ; CN1CCN2c3ccc(Nc4ccc(-c5ccnc6c5ccn6C)c5c4C(=O)NC5)nc3OC[C@@H]2C1]

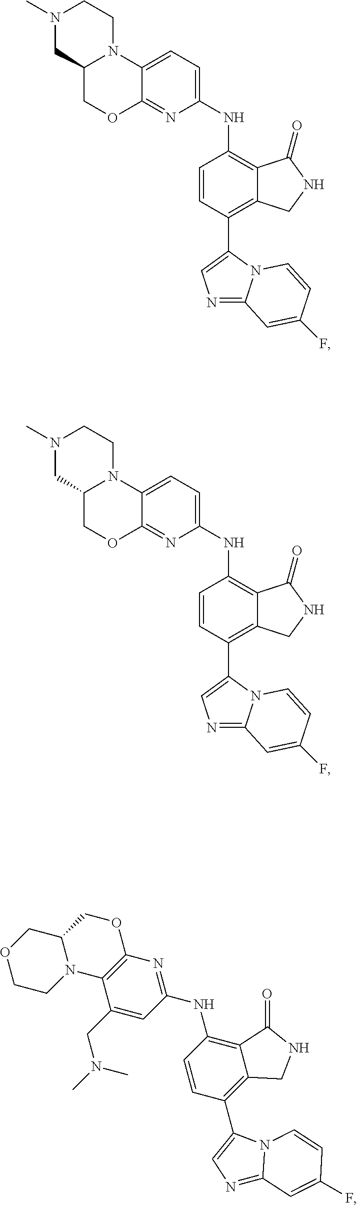 [SMILES: CN1CCN2c3ccc(Nc4ccc(-c5cnc6cc(F)ccn56)c5c4C(=O)NC5)nc3OC[C@H]2C1 ; CN1CCN2c3ccc(Nc4ccc(-c5cnc6cc(F)ccn56)c5c4C(=O)NC5)nc3OC[C@@H]2C1 ; CN(C)Cc1cc(Nc2ccc(-c3cnc4cc(F)ccn34)c3c2C(=O)NC3)nc2c1N1CCOC[C@@H]1CO2]

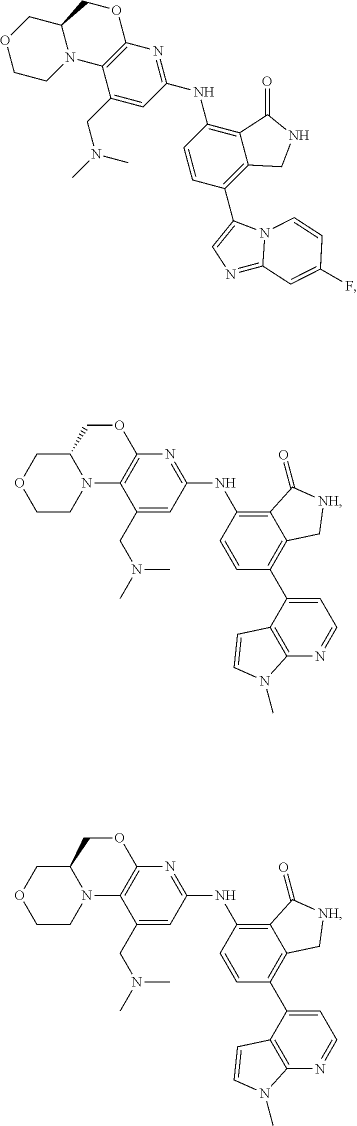 [SMILES: CN(C)Cc1cc(Nc2ccc(-c3cnc4cc(F)ccn34)c3c2C(=O)NC3)nc2c1N1CCOC[C@H]1CO2 ; CN(C)Cc1cc(Nc2ccc(-c3ccnc4c3ccn4C)c3c2C(=O)NC3)nc2c1N1CCOC[C@@H]1CO2 ; CN(C)Cc1cc(Nc2ccc(-c3ccnc4c3ccn4C)c3c2C(=O)NC3)nc2c1N1CCOC[C@H]1CO2]

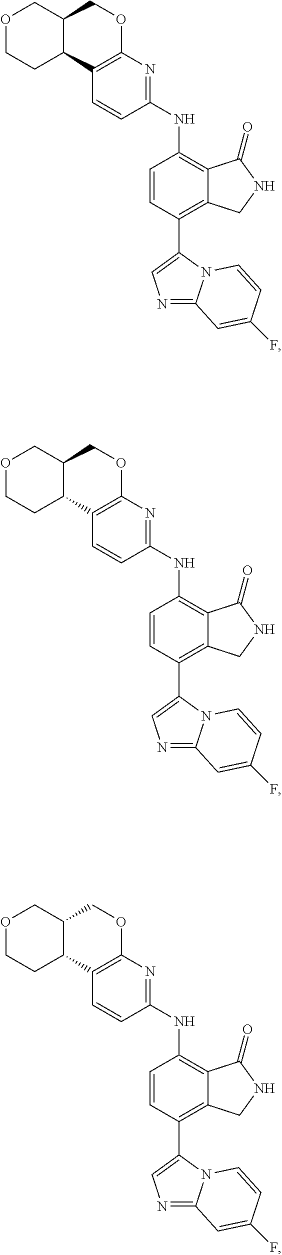 [SMILES: O=C1NCc2c(-c3cnc4cc(F)ccn34)ccc(Nc3ccc4c(n3)OC[C@@H]3COCC[C@H]43)c21 ; O=C1NCc2c(-c3cnc4cc(F)ccn34)ccc(Nc3ccc4c(n3)OC[C@@H]3COCC[C@@H]43)c21 ; O=C1NCc2c(-c3cnc4cc(F)ccn34)ccc(Nc3ccc4c(n3)OC[C@H]3COCC[C@@H]43)c21]

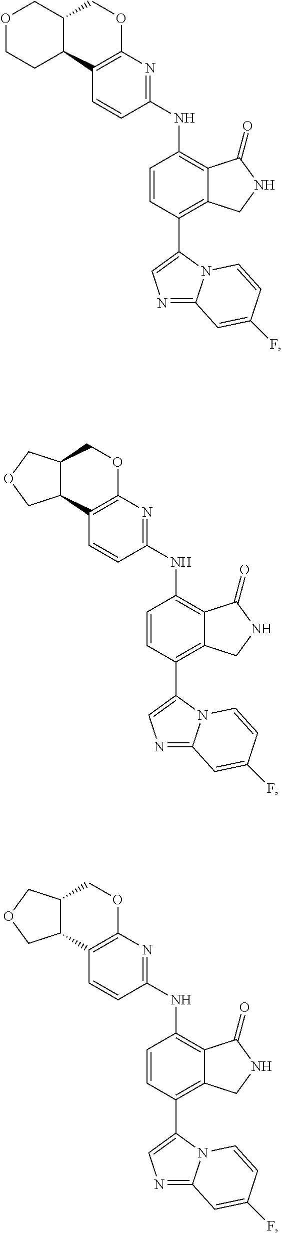 [SMILES: O=C1NCc2c(-c3cnc4cc(F)ccn34)ccc(Nc3ccc4c(n3)OC[C@H]3COCC[C@H]43)c21 ; O=C1NCc2c(-c3cnc4cc(F)ccn34)ccc(Nc3ccc4c(n3)OC[C@@H]3COC[C@H]43)c21 ; O=C1NCc2c(-c3cnc4cc(F)ccn34)ccc(Nc3ccc4c(n3)OC[C@H]3COC[C@@H]43)c21]

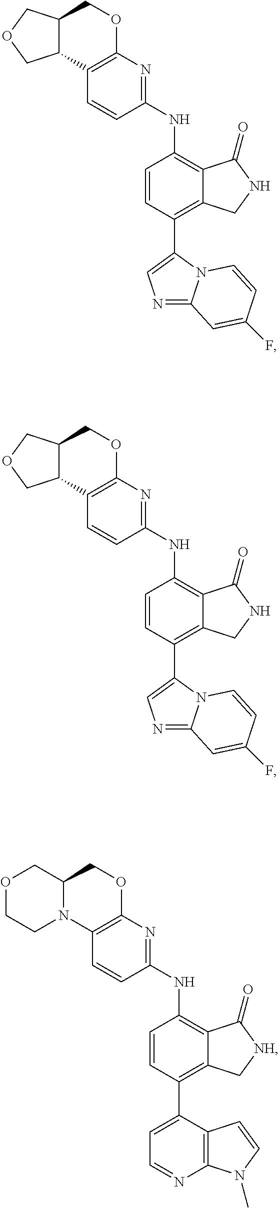 [SMILES: O=C1NCc2c(-c3cnc4cc(F)ccn34)ccc(Nc3ccc4c(n3)OC[C@@H]3COC[C@@H]43)c21 ; O=C1NCc2c(-c3cnc4cc(F)ccn34)ccc(Nc3ccc4c(n3)OC[C@@H]3COC[C@@H]43)c21 ; Cn1ccc2c(-c3ccc(Nc4ccc5c(n4)OC[C@@H]4COCCN54)c4c3CNC4=O)ccnc21]

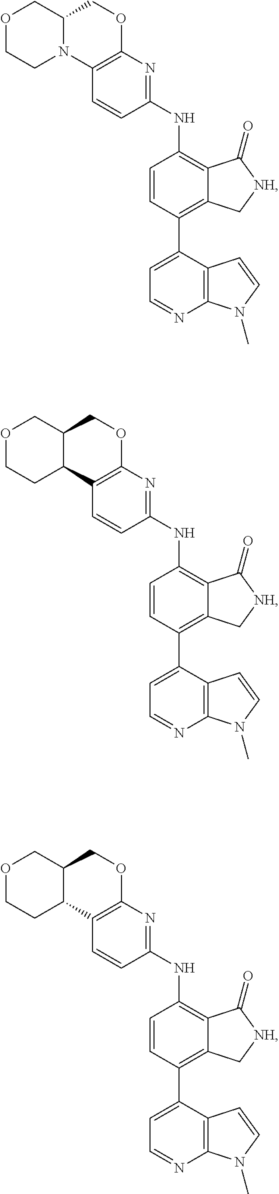 [SMILES: Cn1ccc2c(-c3ccc(Nc4ccc5c(n4)OC[C@H]4COCCN54)c4c3CNC4=O)ccnc21 ; Cn1ccc2c(-c3ccc(Nc4ccc5c(n4)OC[C@@H]4COCC[C@H]54)c4c3CNC4=O)ccnc21 ; Cn1ccc2c(-c3ccc(Nc4ccc5c(n4)OC[C@@H]4COCC[C@@H]54)c4c3CNC4=O)ccnc21]

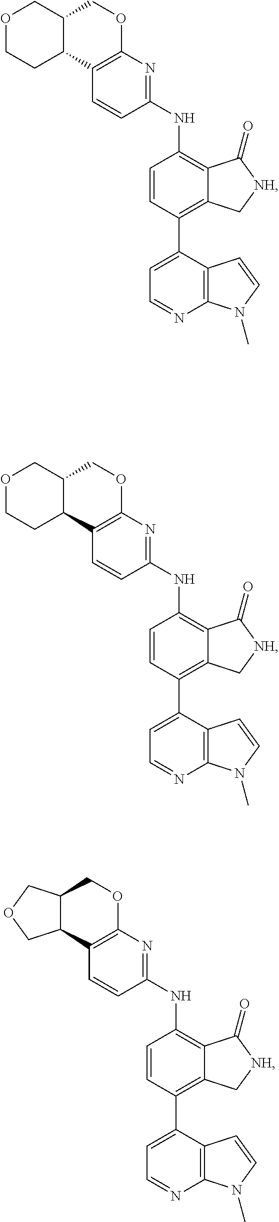 [SMILES: Cn1ccc2c(-c3ccc(Nc4ccc5c(n4)OC[C@H]4COCC[C@@H]54)c4c3CNC4=O)ccnc21 ; Cn1ccc2c(-c3ccc(Nc4ccc5c(n4)OC[C@H]4COCC[C@H]54)c4c3CNC4=O)ccnc21 ; Cn1ccc2c(-c3ccc(Nc4ccc5c(n4)OC[C@@H]4COC[C@H]54)c4c3CNC4=O)ccnc21]

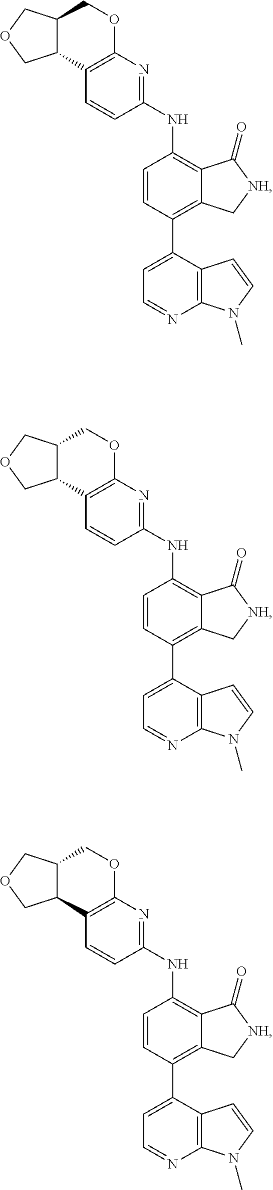 [SMILES: Cn1ccc2c(-c3ccc(Nc4ccc5c(n4)OC[C@@H]4COC[C@@H]54)c4c3CNC4=O)ccnc21 ; Cn1ccc2c(-c3ccc(Nc4ccc5c(n4)OC[C@H]4COC[C@@H]54)c4c3CNC4=O)ccnc21 ; Cn1ccc2c(-c3ccc(Nc4ccc5c(n4)OC[C@H]4COC[C@H]54)c4c3CNC4=O)ccnc21]

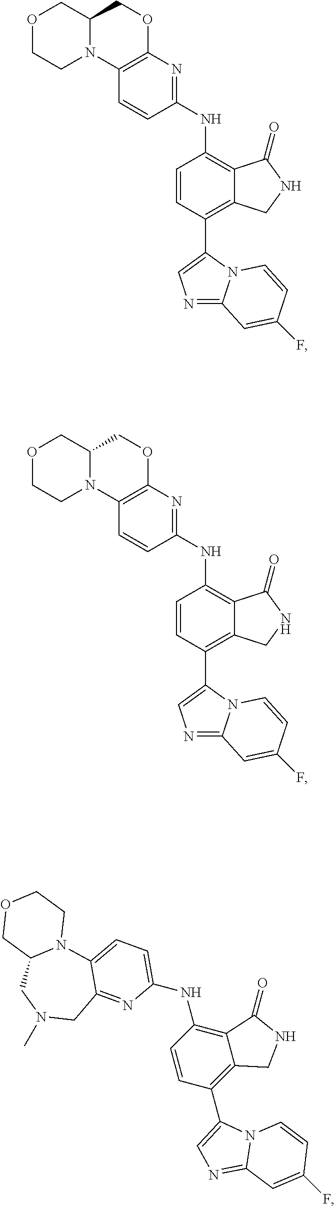 [SMILES: O=C1NCc2c(-c3cnc4cc(F)ccn34)ccc(Nc3ccc4c(n3)OC[C@@H]3COCCN43)c21 ; O=C1NCc2c(-c3cnc4cc(F)ccn34)ccc(Nc3ccc4c(n3)OC[C@H]3COCCN43)c21 ; CN1Cc2nc(Nc3ccc(-c4cnc5cc(F)ccn45)c4c3C(=O)NC4)ccc2N2CCOC[C@H]2C1]

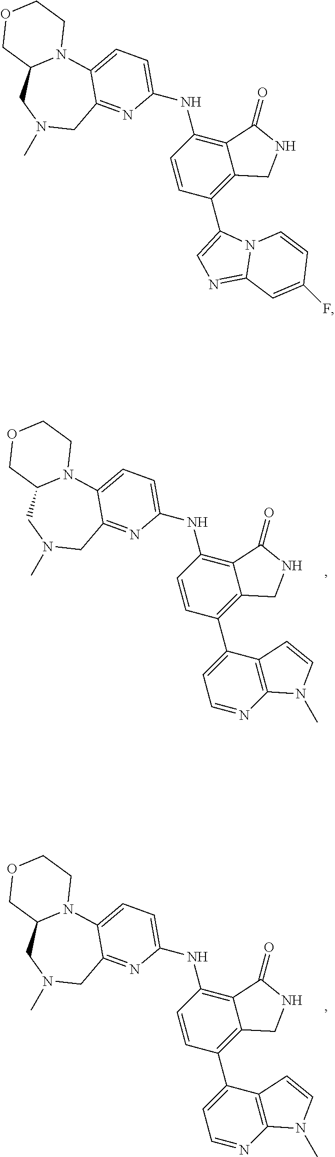 [SMILES: CN1Cc2nc(Nc3ccc(-c4cnc5cc(F)ccn45)c4c3C(=O)NC4)ccc2N2CCOC[C@@H]2C1 ; CN1Cc2nc(Nc3ccc(-c4ccnc5c4ccn5C)c4c3C(=O)NC4)ccc2N2CCOC[C@H]2C1 ; CN1Cc2nc(Nc3ccc(-c4ccnc5c4ccn5C)c4c3C(=O)NC4)ccc2N2CCOC[C@@H]2C1]

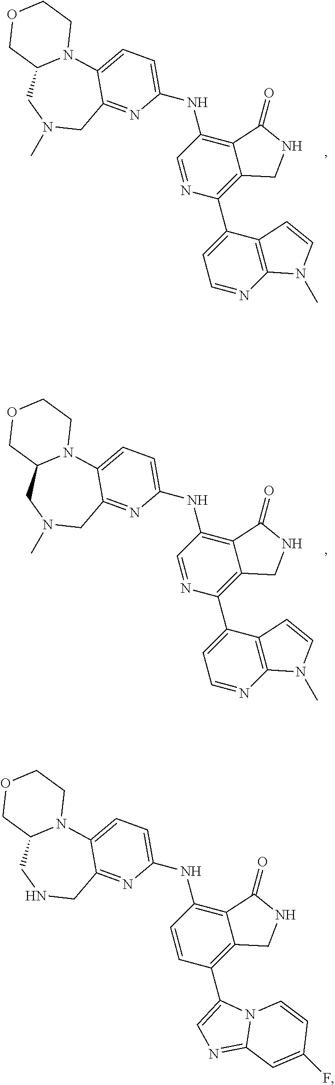 [SMILES: CN1Cc2nc(Nc3cnc(-c4ccnc5c4ccn5C)c4c3C(=O)NC4)ccc2N2CCOC[C@H]2C1 ; CN1Cc2nc(Nc3cnc(-c4ccnc5c4ccn5C)c4c3C(=O)NC4)ccc2N2CCOC[C@@H]2C1 ; O=C1NCc2c(-c3cnc4cc(F)ccn34)ccc(Nc3ccc4c(n3)CNC[C@@H]3COCCN43)c21]

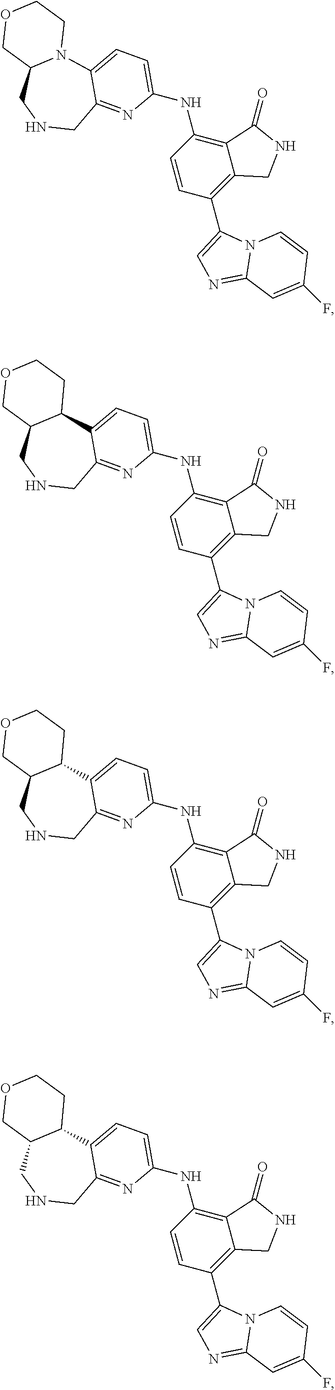 [SMILES: O=C1NCc2c(-c3cnc4cc(F)ccn34)ccc(Nc3ccc4c(n3)CNC[C@H]3COCCN43)c21 ; O=C1NCc2c(-c3cnc4cc(F)ccn34)ccc(Nc3ccc4c(n3)CNC[C@H]3COCC[C@@H]43)c21 ; O=C1NCc2c(-c3cnc4cc(F)ccn34)ccc(Nc3ccc4c(n3)CNC[C@H]3COCC[C@H]43)c21 ; O=C1NCc2c(-c3cnc4cc(F)ccn34)ccc(Nc3ccc4c(n3)CNC[C@@H]3COCC[C@H]43)c21]

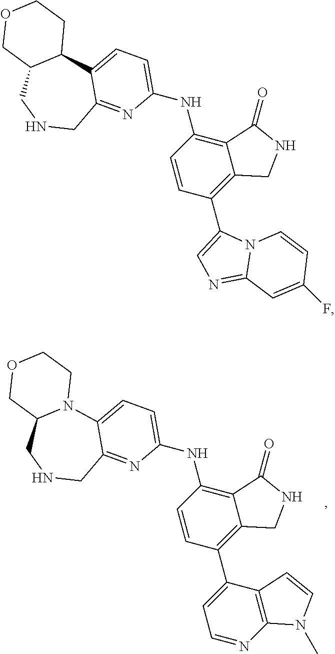 [SMILES: O=C1NCc2c(-c3cnc4cc(F)ccn34)ccc(Nc3ccc4c(n3)CNC[C@@H]3COCC[C@@H]43)c21 ; Cn1ccc2c(-c3ccc(Nc4ccc5c(n4)CNC[C@H]4COCCN54)c4c3CNC4=O)ccnc21]

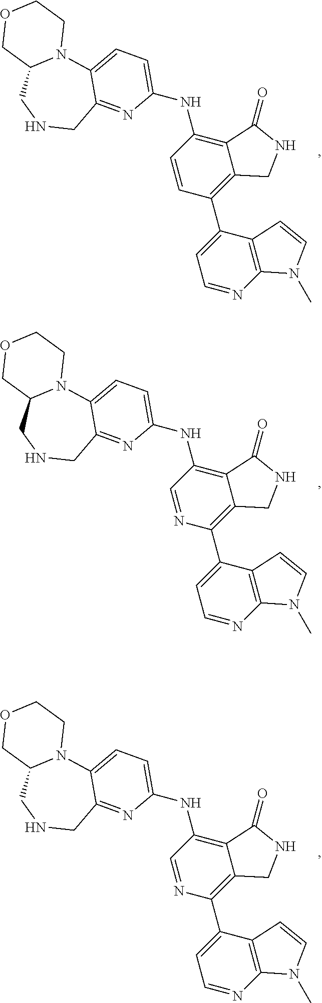 [SMILES: Cn1ccc2c(-c3ccc(Nc4ccc5c(n4)CNC[C@@H]4COCCN54)c4c3CNC4=O)ccnc21 ; Cn1ccc2c(-c3ncc(Nc4ccc5c(n4)CNC[C@H]4COCCN54)c4c3CNC4=O)ccnc21 ; Cn1ccc2c(-c3ncc(Nc4ccc5c(n4)CNC[C@@H]4COCCN54)c4c3CNC4=O)ccnc21]

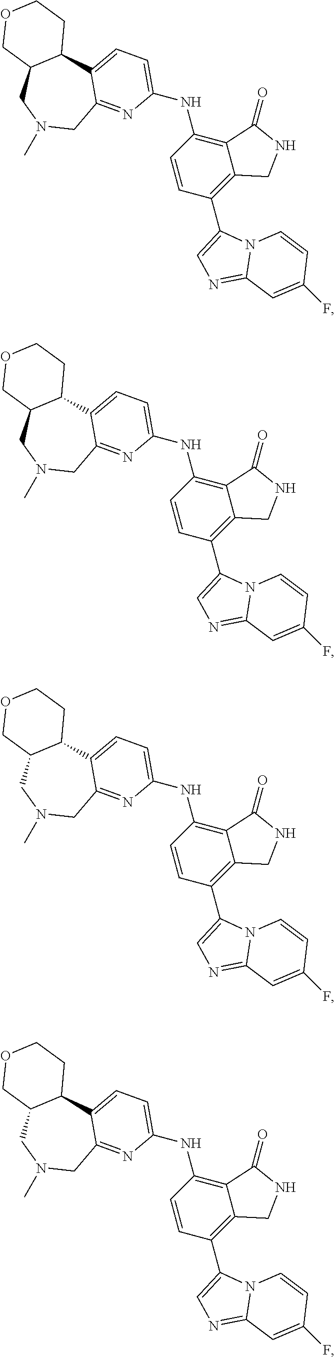 [SMILES: CN1Cc2nc(Nc3ccc(-c4cnc5cc(F)ccn45)c4c3C(=O)NC4)ccc2[C@@H]2CCOC[C@@H]2C1 ; CN1Cc2nc(Nc3ccc(-c4cnc5cc(F)ccn45)c4c3C(=O)NC4)ccc2[C@H]2CCOC[C@@H]2C1 ; CN1Cc2nc(Nc3ccc(-c4cnc5cc(F)ccn45)c4c3C(=O)NC4)ccc2[C@H]2CCOC[C@H]2C1 ; CN1Cc2nc(Nc3ccc(-c4cnc5cc(F)ccn45)c4c3C(=O)NC4)ccc2[C@@H]2CCOC[C@H]2C1]

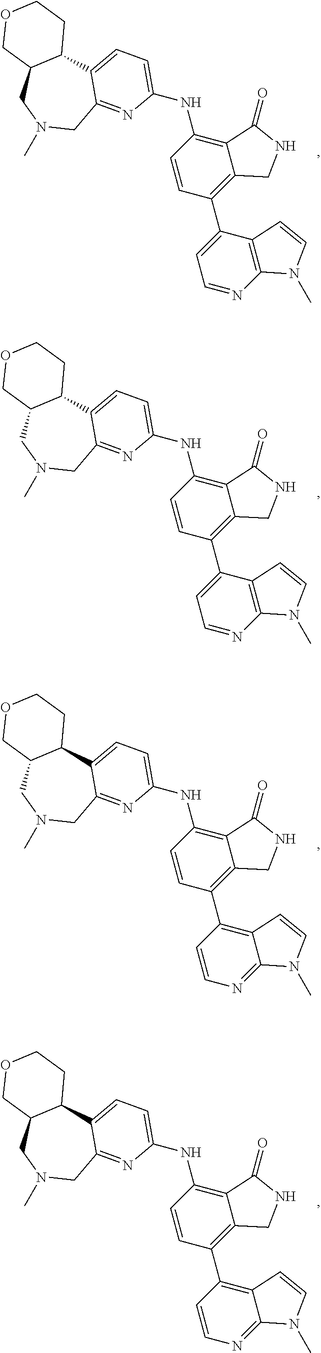 [SMILES: CN1Cc2nc(Nc3ccc(-c4ccnc5c4ccn5C)c4c3C(=O)NC4)ccc2[C@H]2CCOC[C@@H]2C1 ; CN1Cc2nc(Nc3ccc(-c4ccnc5c4ccn5C)c4c3C(=O)NC4)ccc2[C@H]2CCOC[C@H]2C1 ; CN1Cc2nc(Nc3ccc(-c4ccnc5c4ccn5C)c4c3C(=O)NC4)ccc2[C@@H]2CCOC[C@H]2C1 ; CN1Cc2nc(Nc3ccc(-c4ccnc5c4ccn5C)c4c3C(=O)NC4)ccc2[C@@H]2CCOC[C@@H]2C1]

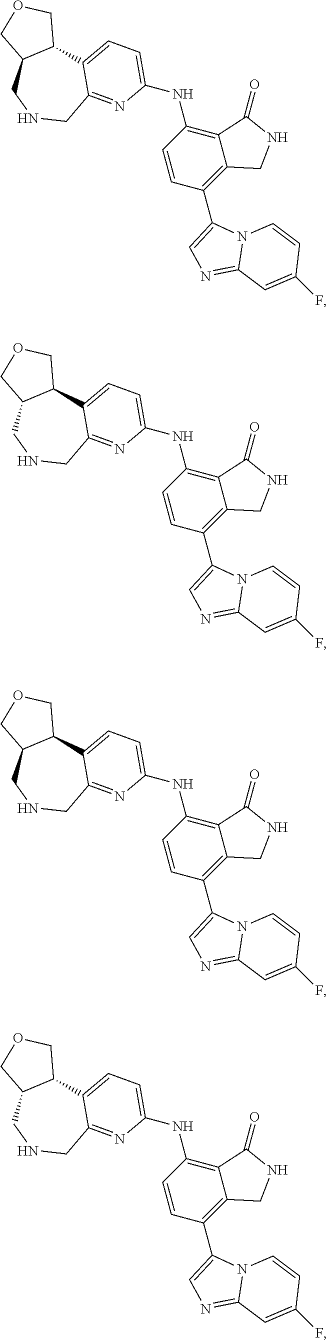 [SMILES: O=C1NCc2c(-c3cnc4cc(F)ccn34)ccc(Nc3ccc4c(n3)CNC[C@H]3COC[C@H]43)c21 ; O=C1NCc2c(-c3cnc4cc(F)ccn34)ccc(Nc3ccc4c(n3)CNC[C@@H]3COC[C@@H]43)c21 ; O=C1NCc2c(-c3cnc4cc(F)ccn34)ccc(Nc3ccc4c(n3)CNC[C@H]3COC[C@@H]43)c21 ; O=C1NCc2c(-c3cnc4cc(F)ccn34)ccc(Nc3ccc4c(n3)CNC[C@@H]3COC[C@H]43)c21]

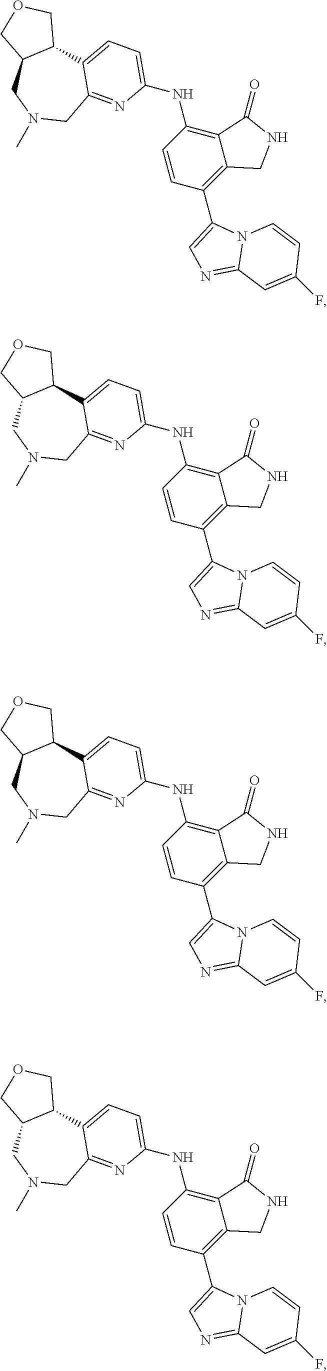 [SMILES: CN1Cc2nc(Nc3ccc(-c4cnc5cc(F)ccn45)c4c3C(=O)NC4)ccc2[C@H]2COC[C@@H]2C1 ; CN1Cc2nc(Nc3ccc(-c4cnc5cc(F)ccn45)c4c3C(=O)NC4)ccc2[C@@H]2COC[C@H]2C1 ; CN1Cc2nc(Nc3ccc(-c4cnc5cc(F)ccn45)c4c3C(=O)NC4)ccc2[C@@H]2COC[C@@H]2C1 ; CN1Cc2nc(Nc3ccc(-c4cnc5cc(F)ccn45)c4c3C(=O)NC4)ccc2[C@H]2COC[C@H]2C1]

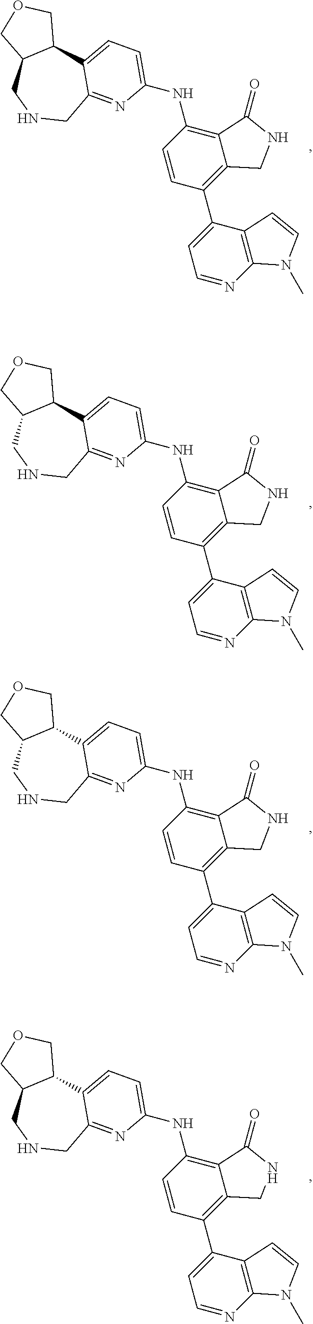 [SMILES: Cn1ccc2c(-c3ccc(Nc4ccc5c(n4)CNC[C@H]4COC[C@@H]54)c4c3CNC4=O)ccnc21 ; Cn1ccc2c(-c3ccc(Nc4ccc5c(n4)CNC[C@@H]4COC[C@@H]54)c4c3CNC4=O)ccnc21 ; Cn1ccc2c(-c3ccc(Nc4ccc5c(n4)CNC[C@@H]4COC[C@H]54)c4c3CNC4=O)ccnc21 ; Cn1ccc2c(-c3ccc(Nc4ccc5c(n4)CNC[C@H]4COC[C@H]54)c4c3CNC4=O)ccnc21]

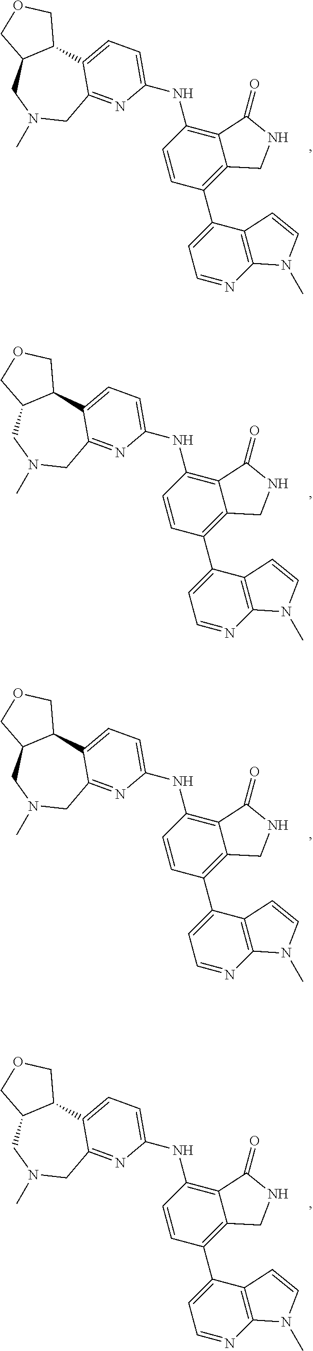 [SMILES: CN1Cc2nc(Nc3ccc(-c4ccnc5c4ccn5C)c4c3C(=O)NC4)ccc2[C@H]2COC[C@@H]2C1 ; CN1Cc2nc(Nc3ccc(-c4ccnc5c4ccn5C)c4c3C(=O)NC4)ccc2[C@@H]2COC[C@H]2C1 ; CN1Cc2nc(Nc3ccc(-c4ccnc5c4ccn5C)c4c3C(=O)NC4)ccc2[C@@H]2COC[C@@H]2C1 ; CN1Cc2nc(Nc3ccc(-c4ccnc5c4ccn5C)c4c3C(=O)NC4)ccc2[C@H]2COC[C@H]2C1]

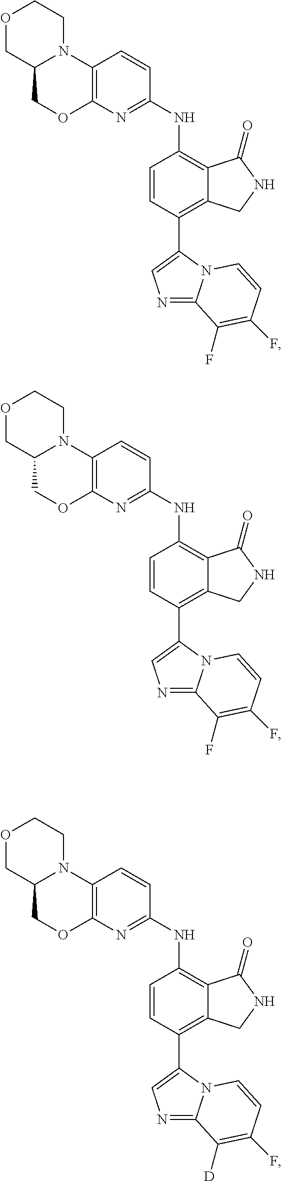 [SMILES: O=C1NCc2c(-c3cnc4c(F)c(F)ccn34)ccc(Nc3ccc4c(n3)OC[C@H]3COCCN43)c21 ; O=C1NCc2c(-c3cnc4c(F)c(F)ccn34)ccc(Nc3ccc4c(n3)OC[C@@H]3COCCN43)c21 ; [2H]c1c(F)ccn2c(-c3ccc(Nc4ccc5c(n4)OC[C@H]4COCCN54)c4c3CNC4=O)cnc12]

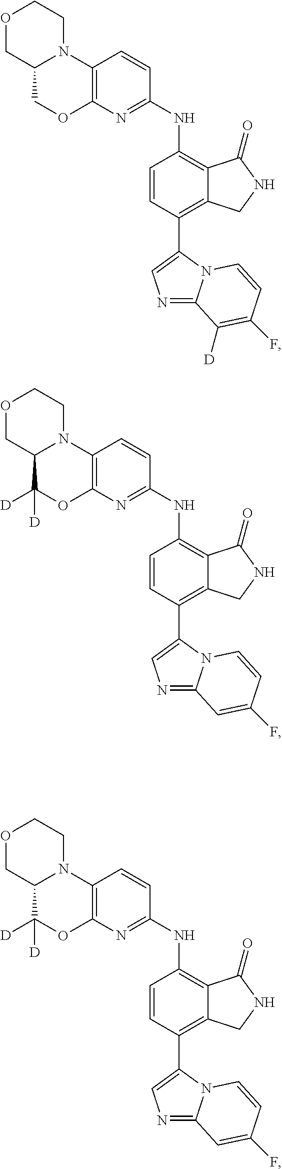 [SMILES: [2H]c1c(F)ccn2c(-c3ccc(Nc4ccc5c(n4)OC[C@@H]4COCCN54)c4c3CNC4=O)cnc12 ; [2H]C1([2H])Oc2nc(Nc3ccc(-c4cnc5cc(F)ccn45)c4c3C(=O)NC4)ccc2N2CCOC[C@@H]21 ; [2H]C1([2H])Oc2nc(Nc3ccc(-c4cnc5cc(F)ccn45)c4c3C(=O)NC4)ccc2N2CCOC[C@H]21]

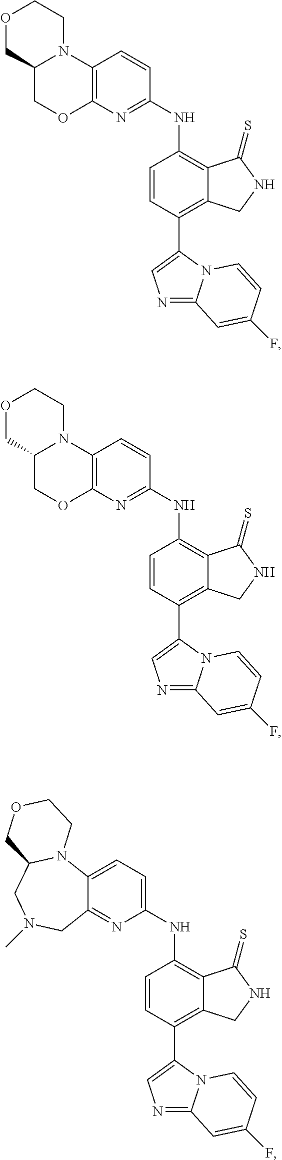 [SMILES: Fc1ccn2c(-c3ccc(Nc4ccc5c(n4)OC[C@H]4COCCN54)c4c3CNC4=S)cnc2c1 ; Fc1ccn2c(-c3ccc(Nc4ccc5c(n4)OC[C@@H]4COCCN54)c4c3CNC4=S)cnc2c1 ; CN1Cc2nc(Nc3ccc(-c4cnc5cc(F)ccn45)c4c3C(=S)NC4)ccc2N2CCOC[C@@H]2C1]

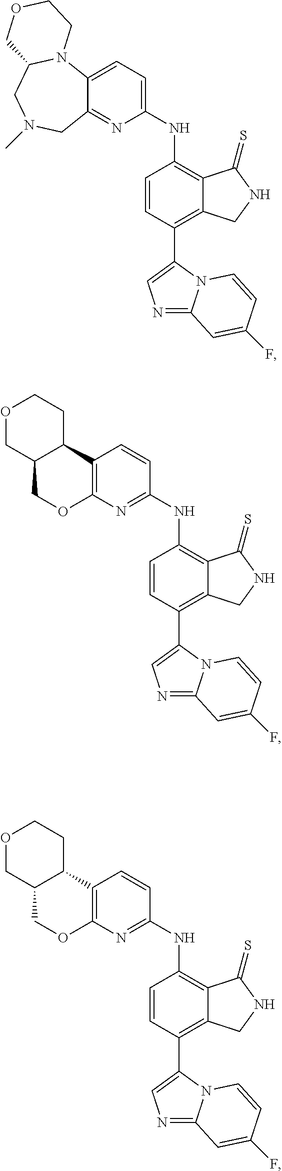 [SMILES: CN1Cc2nc(Nc3ccc(-c4cnc5cc(F)ccn45)c4c3C(=S)NC4)ccc2N2CCOC[C@H]2C1 ; Fc1ccn2c(-c3ccc(Nc4ccc5c(n4)OC[C@H]4COCC[C@@H]54)c4c3CNC4=S)cnc2c1 ; Fc1ccn2c(-c3ccc(Nc4ccc5c(n4)OC[C@@H]4COCC[C@H]54)c4c3CNC4=S)cnc2c1]

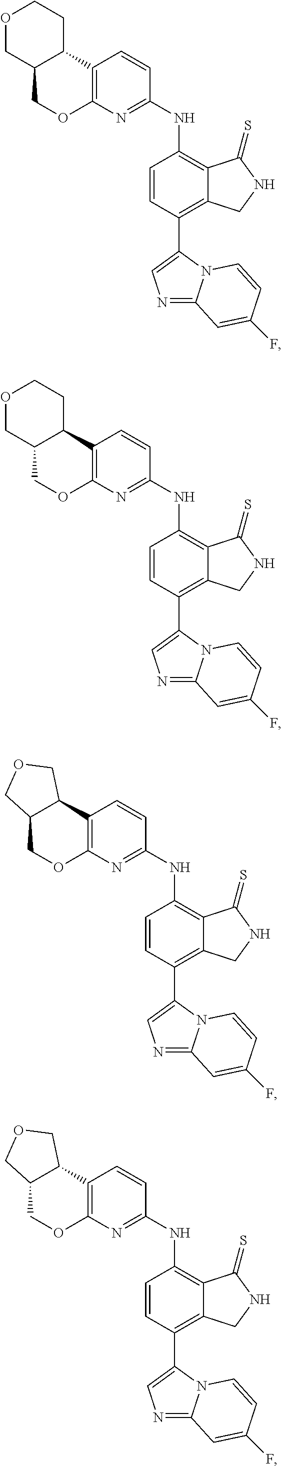 [SMILES: Fc1ccn2c(-c3ccc(Nc4ccc5c(n4)OC[C@H]4COCC[C@H]54)c4c3CNC4=S)cnc2c1 ; Fc1ccn2c(-c3ccc(Nc4ccc5c(n4)OC[C@@H]4COCC[C@@H]54)c4c3CNC4=S)cnc2c1 ; Fc1ccn2c(-c3ccc(Nc4ccc5c(n4)OC[C@H]4COC[C@@H]54)c4c3CNC4=S)cnc2c1 ; Fc1ccn2c(-c3ccc(Nc4ccc5c(n4)OC[C@@H]4COC[C@H]54)c4c3CNC4=S)cnc2c1]

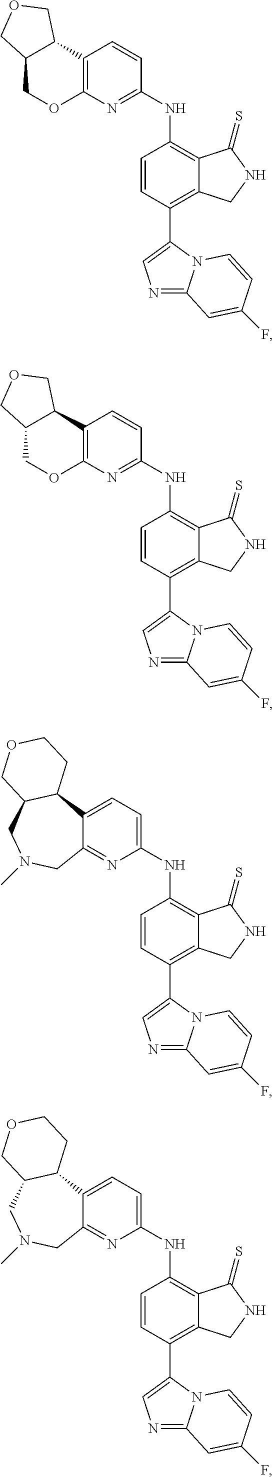 [SMILES: Fc1ccn2c(-c3ccc(Nc4ccc5c(n4)OC[C@H]4COC[C@H]54)c4c3CNC4=S)cnc2c1 ; Fc1ccn2c(-c3ccc(Nc4ccc5c(n4)OC[C@@H]4COC[C@@H]54)c4c3CNC4=S)cnc2c1 ; CN1Cc2nc(Nc3ccc(-c4cnc5cc(F)ccn45)c4c3C(=S)NC4)ccc2[C@@H]2CCOC[C@@H]2C1 ; CN1Cc2nc(Nc3ccc(-c4cnc5cc(F)ccn45)c4c3C(=S)NC4)ccc2[C@H]2CCOC[C@H]2C1]

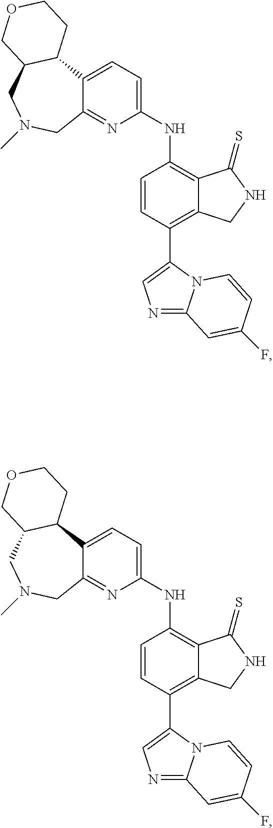 [SMILES: CN1Cc2nc(Nc3ccc(-c4cnc5cc(F)ccn45)c4c3C(=S)NC4)ccc2[C@H]2CCOC[C@@H]2C1 ; CN1Cc2nc(Nc3ccc(-c4cnc5cc(F)ccn45)c4c3C(=S)NC4)ccc2[C@@H]2CCOC[C@H]2C1]

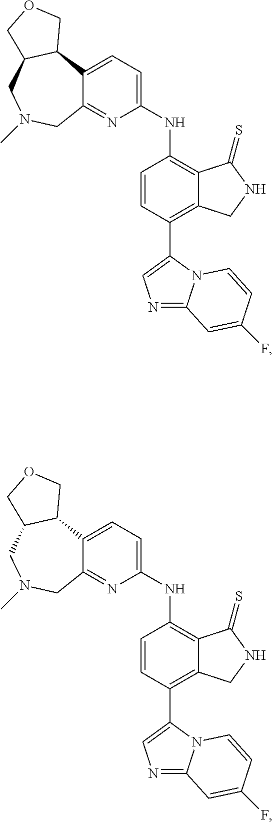 [SMILES: CN1Cc2nc(Nc3ccc(-c4cnc5cc(F)ccn45)c4c3C(=S)NC4)ccc2[C@@H]2COC[C@@H]2C1 ; CN1Cc2nc(Nc3ccc(-c4cnc5cc(F)ccn45)c4c3C(=S)NC4)ccc2[C@H]2COC[C@H]2C1]

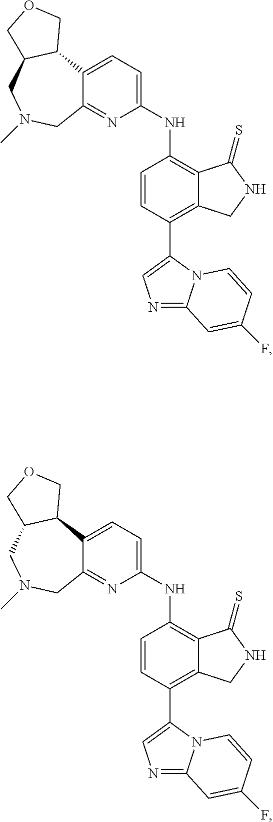 [SMILES: CN1Cc2nc(Nc3ccc(-c4cnc5cc(F)ccn45)c4c3C(=S)NC4)ccc2[C@H]2COC[C@@H]2C1 ; CN1Cc2nc(Nc3ccc(-c4cnc5cc(F)ccn45)c4c3C(=S)NC4)ccc2[C@@H]2COC[C@H]2C1]

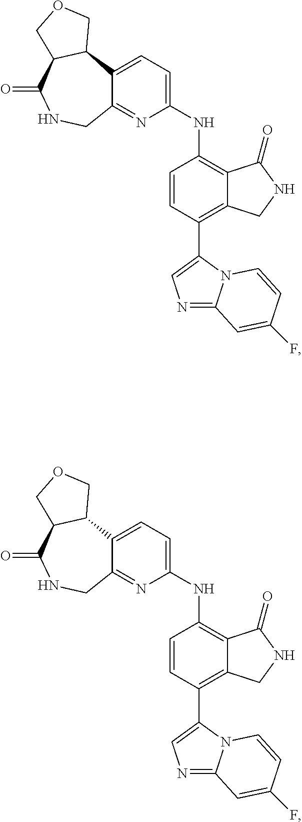 [SMILES: O=C1NCc2c(-c3cnc4cc(F)ccn34)ccc(Nc3ccc4c(n3)CNC(=O)[C@H]3COC[C@@H]43)c21 ; O=C1NCc2c(-c3cnc4cc(F)ccn34)ccc(Nc3ccc4c(n3)CNC(=O)[C@H]3COC[C@H]43)c21]

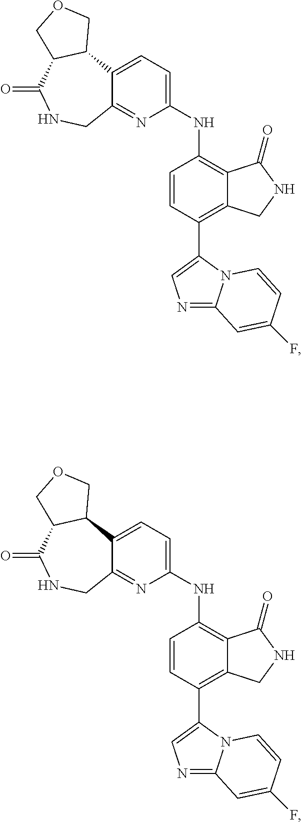 [SMILES: O=C1NCc2c(-c3cnc4cc(F)ccn34)ccc(Nc3ccc4c(n3)CNC(=O)[C@@H]3COC[C@H]43)c21 ; O=C1NCc2c(-c3cnc4cc(F)ccn34)ccc(Nc3ccc4c(n3)CNC(=O)[C@@H]3COC[C@@H]43)c21]

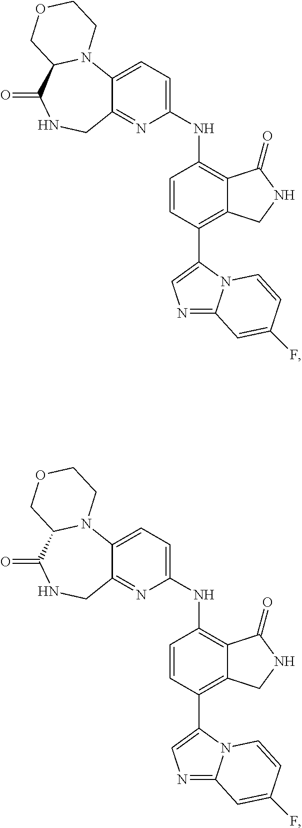 [SMILES: O=C1NCc2c(-c3cnc4cc(F)ccn34)ccc(Nc3ccc4c(n3)CNC(=O)[C@H]3COCCN43)c21 ; O=C1NCc2c(-c3cnc4cc(F)ccn34)ccc(Nc3ccc4c(n3)CNC(=O)[C@@H]3COCCN43)c21]

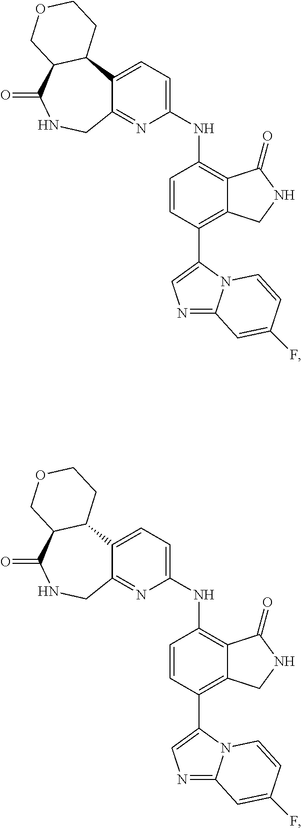 [SMILES: O=C1NCc2c(-c3cnc4cc(F)ccn34)ccc(Nc3ccc4c(n3)CNC(=O)[C@H]3COCC[C@@H]43)c21 ; O=C1NCc2c(-c3cnc4cc(F)ccn34)ccc(Nc3ccc4c(n3)CNC(=O)[C@H]3COCC[C@H]43)c21]

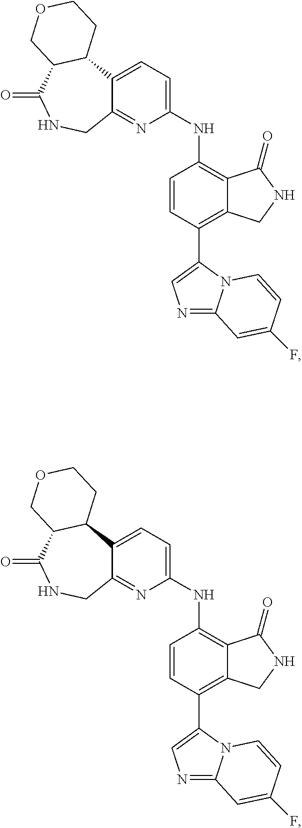 [SMILES: O=C1NCc2c(-c3cnc4cc(F)ccn34)ccc(Nc3ccc4c(n3)CNC(=O)[C@@H]3COCC[C@H]43)c21 ; O=C1NCc2c(-c3cnc4cc(F)ccn34)ccc(Nc3ccc4c(n3)CNC(=O)[C@@H]3COCC[C@@H]43)c21]

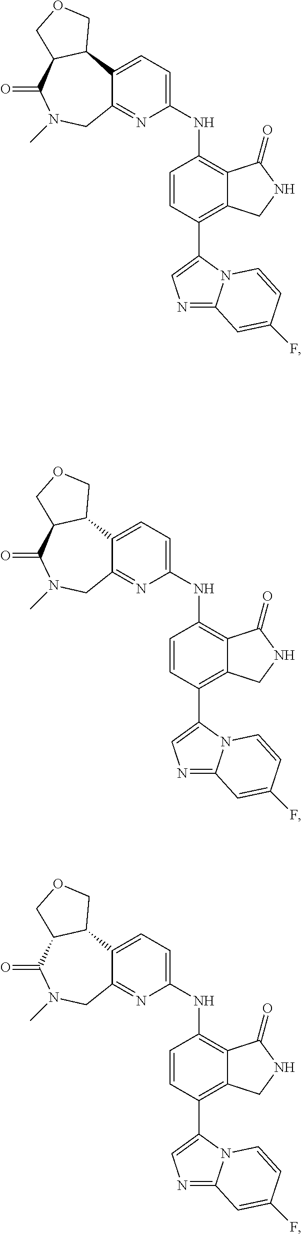 [SMILES: CN1Cc2nc(Nc3ccc(-c4cnc5cc(F)ccn45)c4c3C(=O)NC4)ccc2[C@@H]2COC[C@@H]2C1=O ; CN1Cc2nc(Nc3ccc(-c4cnc5cc(F)ccn45)c4c3C(=O)NC4)ccc2[C@H]2COC[C@@H]2C1=O ; CN1Cc2nc(Nc3ccc(-c4cnc5cc(F)ccn45)c4c3C(=O)NC4)ccc2[C@H]2COC[C@H]2C1=O]

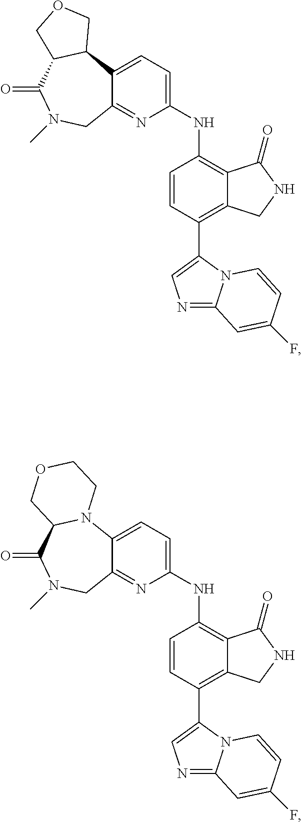 [SMILES: CN1Cc2nc(Nc3ccc(-c4cnc5cc(F)ccn45)c4c3C(=O)NC4)ccc2[C@@H]2COC[C@H]2C1=O ; CN1Cc2nc(Nc3ccc(-c4cnc5cc(F)ccn45)c4c3C(=O)NC4)ccc2N2CCOC[C@@H]2C1=O]

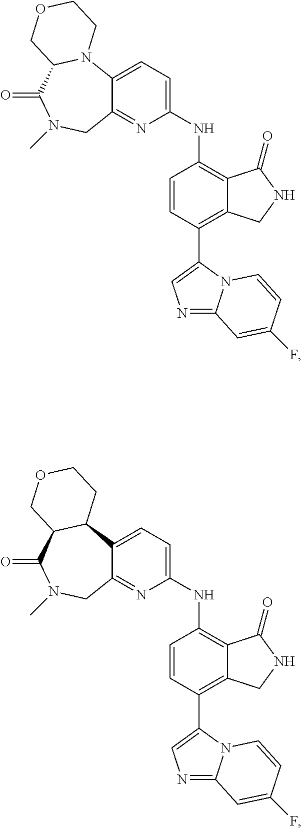 [SMILES: CN1Cc2nc(Nc3ccc(-c4cnc5cc(F)ccn45)c4c3C(=O)NC4)ccc2N2CCOC[C@H]2C1=O ; CN1Cc2nc(Nc3ccc(-c4cnc5cc(F)ccn45)c4c3C(=O)NC4)ccc2[C@@H]2CCOC[C@@H]2C1=O]

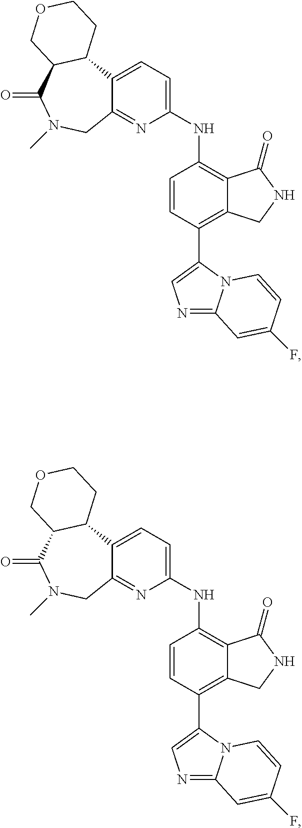 [SMILES: CN1Cc2nc(Nc3ccc(-c4cnc5cc(F)ccn45)c4c3C(=O)NC4)ccc2[C@H]2CCOC[C@@H]2C1=O ; CN1Cc2nc(Nc3ccc(-c4cnc5cc(F)ccn45)c4c3C(=O)NC4)ccc2[C@H]2CCOC[C@H]2C1=O]

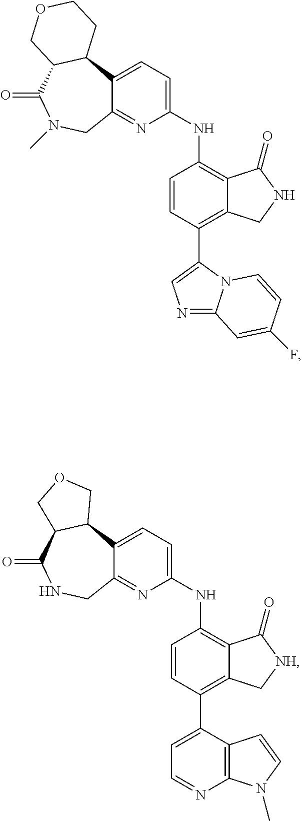 [SMILES: CN1Cc2nc(Nc3ccc(-c4cnc5cc(F)ccn45)c4c3C(=O)NC4)ccc2[C@@H]2CCOC[C@H]2C1=O ; Cn1ccc2c(-c3ccc(Nc4ccc5c(n4)CNC(=O)[C@H]4COC[C@@H]54)c4c3CNC4=O)ccnc21]

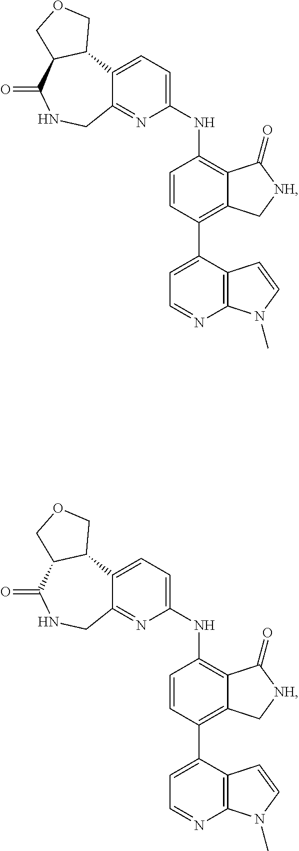 [SMILES: Cn1ccc2c(-c3ccc(Nc4ccc5c(n4)CNC(=O)[C@H]4COC[C@H]54)c4c3CNC4=O)ccnc21 ; Cn1ccc2c(-c3ccc(Nc4ccc5c(n4)CNC(=O)[C@@H]4COC[C@H]54)c4c3CNC4=O)ccnc21]

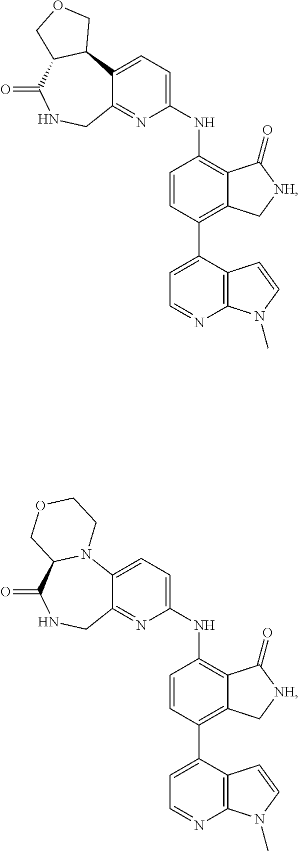 [SMILES: Cn1ccc2c(-c3ccc(Nc4ccc5c(n4)CNC(=O)[C@@H]4COC[C@@H]54)c4c3CNC4=O)ccnc21 ; Cn1ccc2c(-c3ccc(Nc4ccc5c(n4)CNC(=O)[C@H]4COCCN54)c4c3CNC4=O)ccnc21]

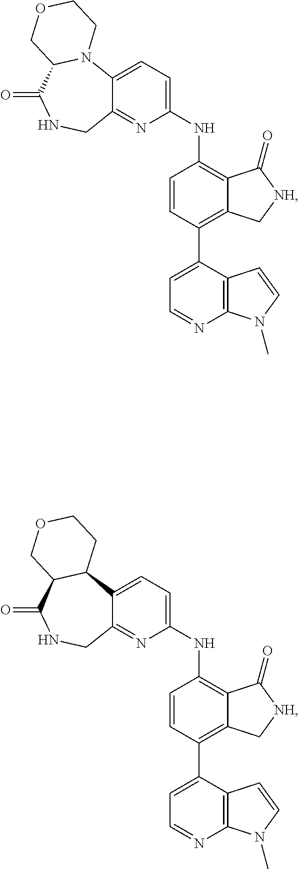 [SMILES: Cn1ccc2c(-c3ccc(Nc4ccc5c(n4)CNC(=O)[C@@H]4COCCN54)c4c3CNC4=O)ccnc21 ; Cn1ccc2c(-c3ccc(Nc4ccc5c(n4)CNC(=O)[C@H]4COCC[C@@H]54)c4c3CNC4=O)ccnc21]

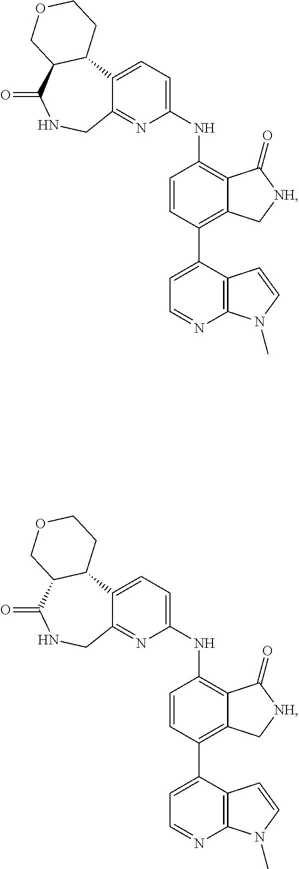 [SMILES: Cn1ccc2c(-c3ccc(Nc4ccc5c(n4)CNC(=O)[C@H]4COCC[C@H]54)c4c3CNC4=O)ccnc21 ; Cn1ccc2c(-c3ccc(Nc4ccc5c(n4)CNC(=O)[C@@H]4COCC[C@H]54)c4c3CNC4=O)ccnc21]

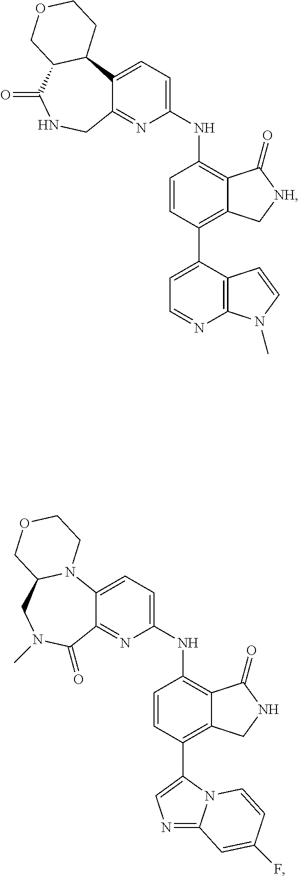 [SMILES: Cn1ccc2c(-c3ccc(Nc4ccc5c(n4)CNC(=O)[C@@H]4COCC[C@@H]54)c4c3CNC4=O)ccnc21 ; CN1C[C@H]2COCCN2c2ccc(Nc3ccc(-c4cnc5cc(F)ccn45)c4c3C(=O)NC4)nc2C1=O]

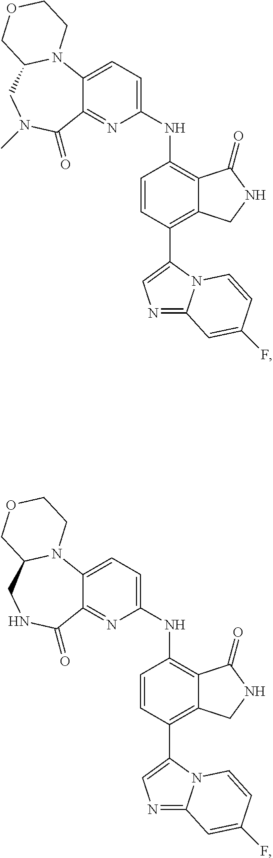 [SMILES: CN1C[C@@H]2COCCN2c2ccc(Nc3ccc(-c4cnc5cc(F)ccn45)c4c3C(=O)NC4)nc2C1=O ; O=C1NC[C@H]2COCCN2c2ccc(Nc3ccc(-c4cnc5cc(F)ccn45)c4c3C(=O)NC4)nc21]

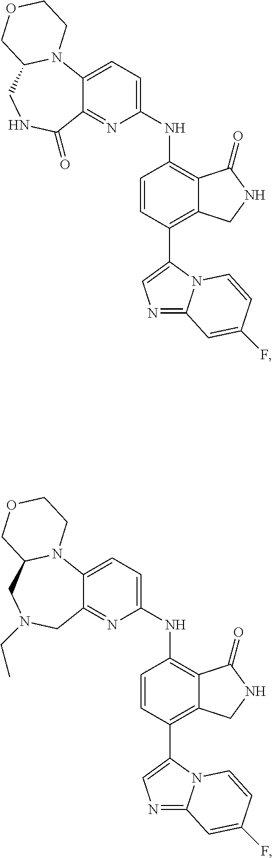 [SMILES: O=C1NC[C@@H]2COCCN2c2ccc(Nc3ccc(-c4cnc5cc(F)ccn45)c4c3C(=O)NC4)nc21 ; CCN1Cc2nc(Nc3ccc(-c4cnc5cc(F)ccn45)c4c3C(=O)NC4)ccc2N2CCOC[C@@H]2C1]

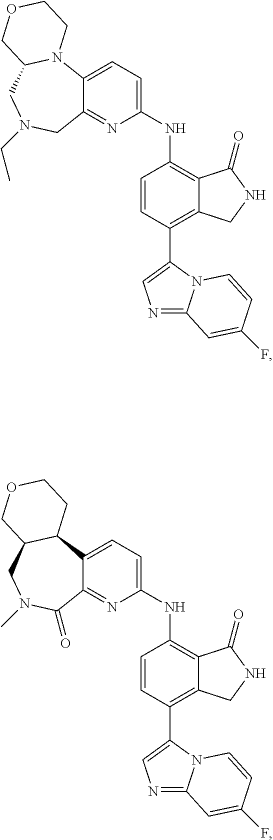 [SMILES: CCN1Cc2nc(Nc3ccc(-c4cnc5cc(F)ccn45)c4c3C(=O)NC4)ccc2N2CCOC[C@H]2C1 ; CN1C[C@H]2COCC[C@H]2c2ccc(Nc3ccc(-c4cnc5cc(F)ccn45)c4c3C(=O)NC4)nc2C1=O]

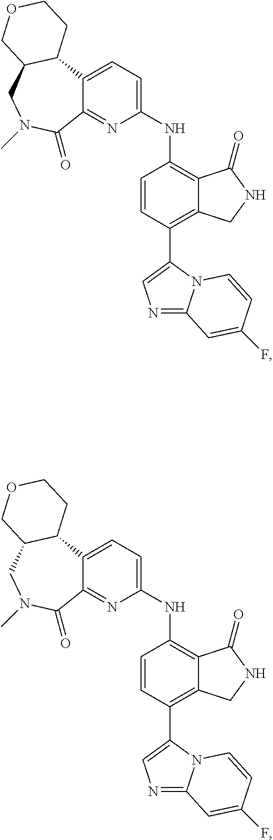 [SMILES: CN1C[C@H]2COCC[C@@H]2c2ccc(Nc3ccc(-c4cnc5cc(F)ccn45)c4c3C(=O)NC4)nc2C1=O ; CN1C[C@@H]2COCC[C@@H]2c2ccc(Nc3ccc(-c4cnc5cc(F)ccn45)c4c3C(=O)NC4)nc2C1=O]

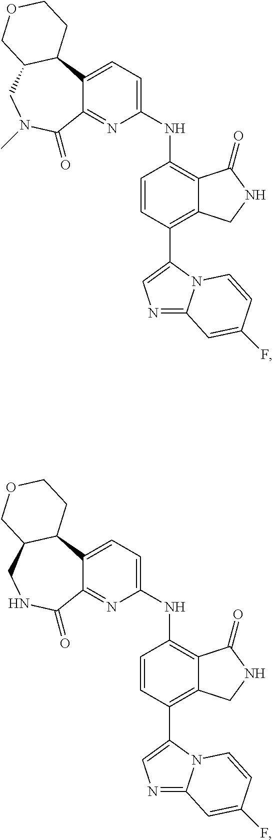 [SMILES: CN1C[C@@H]2COCC[C@H]2c2ccc(Nc3ccc(-c4cnc5cc(F)ccn45)c4c3C(=O)NC4)nc2C1=O ; O=C1NC[C@H]2COCC[C@H]2c2ccc(Nc3ccc(-c4cnc5cc(F)ccn45)c4c3C(=O)NC4)nc21]

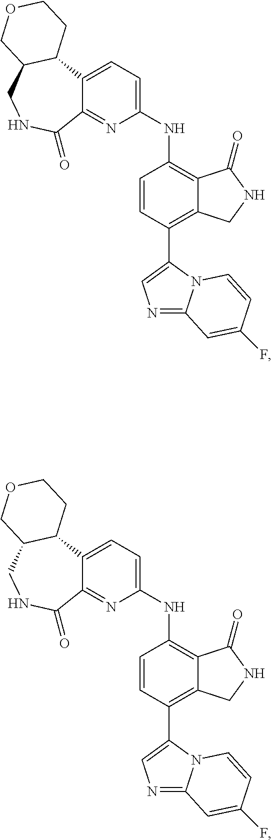 [SMILES: O=C1NC[C@H]2COCC[C@@H]2c2ccc(Nc3ccc(-c4cnc5cc(F)ccn45)c4c3C(=O)NC4)nc21 ; O=C1NC[C@@H]2COCC[C@@H]2c2ccc(Nc3ccc(-c4cnc5cc(F)ccn45)c4c3C(=O)NC4)nc21]

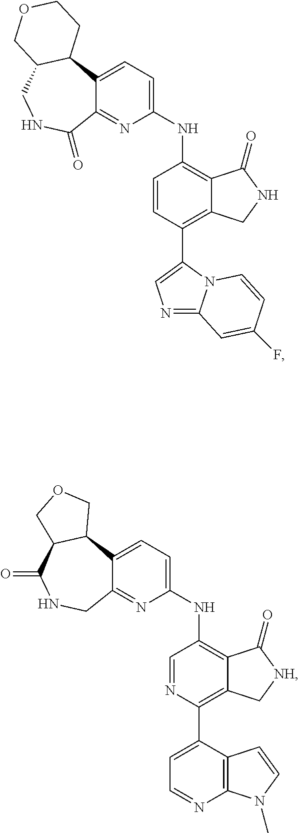 [SMILES: O=C1NC[C@@H]2COCC[C@H]2c2ccc(Nc3ccc(-c4cnc5cc(F)ccn45)c4c3C(=O)NC4)nc21 ; Cn1ccc2c(-c3ncc(Nc4ccc5c(n4)CNC(=O)[C@H]4COC[C@@H]54)c4c3CNC4=O)ccnc21]

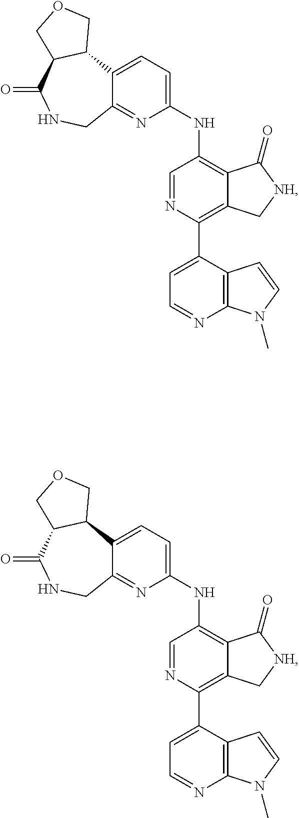 [SMILES: Cn1ccc2c(-c3ncc(Nc4ccc5c(n4)CNC(=O)[C@H]4COC[C@H]54)c4c3CNC4=O)ccnc21 ; Cn1ccc2c(-c3ncc(Nc4ccc5c(n4)CNC(=O)[C@@H]4COC[C@@H]54)c4c3CNC4=O)ccnc21]

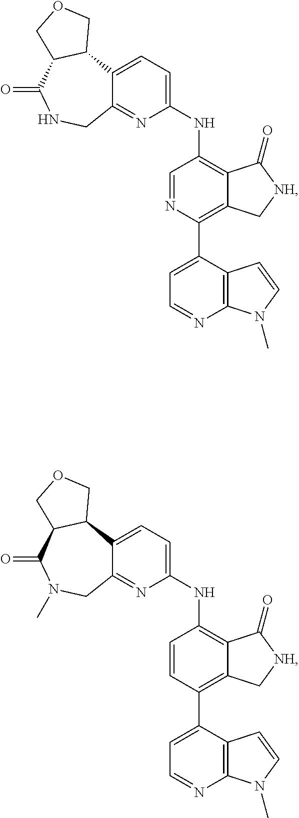 [SMILES: Cn1ccc2c(-c3ncc(Nc4ccc5c(n4)CNC(=O)[C@@H]4COC[C@H]54)c4c3CNC4=O)ccnc21 ; CN1Cc2nc(Nc3ccc(-c4ccnc5c4ccn5C)c4c3C(=O)NC4)ccc2[C@@H]2COC[C@@H]2C1=O]

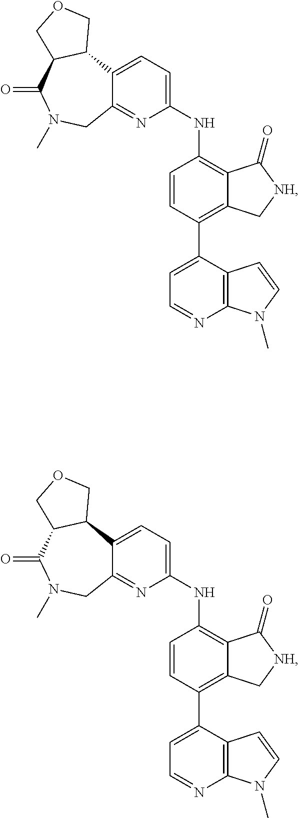 [SMILES: CN1Cc2nc(Nc3ccc(-c4ccnc5c4ccn5C)c4c3C(=O)NC4)ccc2[C@H]2COC[C@@H]2C1=O ; CN1Cc2nc(Nc3ccc(-c4ccnc5c4ccn5C)c4c3C(=O)NC4)ccc2[C@@H]2COC[C@H]2C1=O]

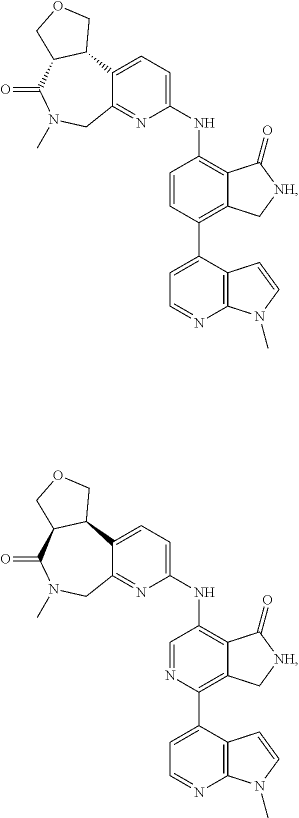 [SMILES: CN1Cc2nc(Nc3ccc(-c4ccnc5c4ccn5C)c4c3C(=O)NC4)ccc2[C@H]2COC[C@H]2C1=O ; CN1Cc2nc(Nc3cnc(-c4ccnc5c4ccn5C)c4c3C(=O)NC4)ccc2[C@@H]2COC[C@@H]2C1=O]

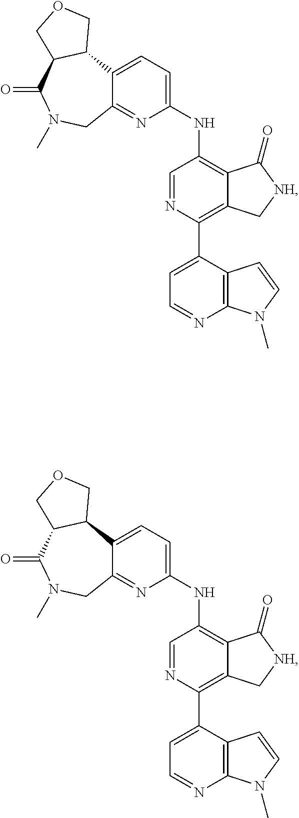 [SMILES: CN1Cc2nc(Nc3cnc(-c4ccnc5c4ccn5C)c4c3C(=O)NC4)ccc2[C@H]2COC[C@@H]2C1=O ; CN1Cc2nc(Nc3cnc(-c4ccnc5c4ccn5C)c4c3C(=O)NC4)ccc2[C@@H]2COC[C@H]2C1=O]

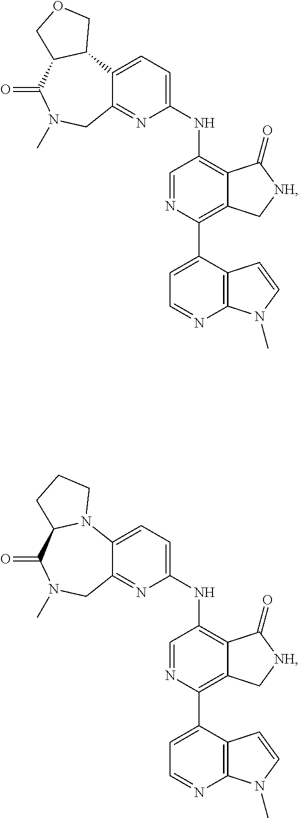 [SMILES: CN1Cc2nc(Nc3cnc(-c4ccnc5c4ccn5C)c4c3C(=O)NC4)ccc2[C@H]2COC[C@H]2C1=O ; CN1Cc2nc(Nc3cnc(-c4ccnc5c4ccn5C)c4c3C(=O)NC4)ccc2N2CCC[C@@H]2C1=O]

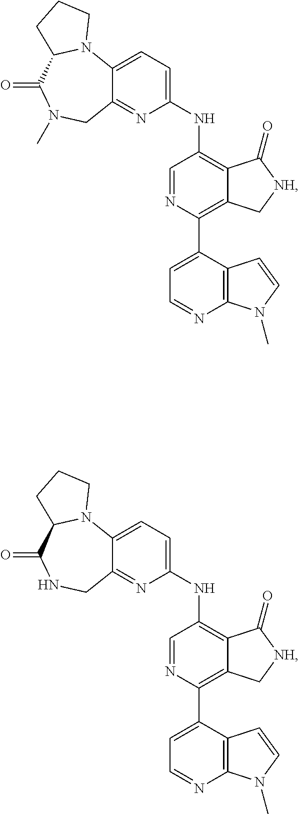 [SMILES: CN1Cc2nc(Nc3cnc(-c4ccnc5c4ccn5C)c4c3C(=O)NC4)ccc2N2CCC[C@H]2C1=O ; Cn1ccc2c(-c3ncc(Nc4ccc5c(n4)CNC(=O)[C@H]4CCCN54)c4c3CNC4=O)ccnc21]

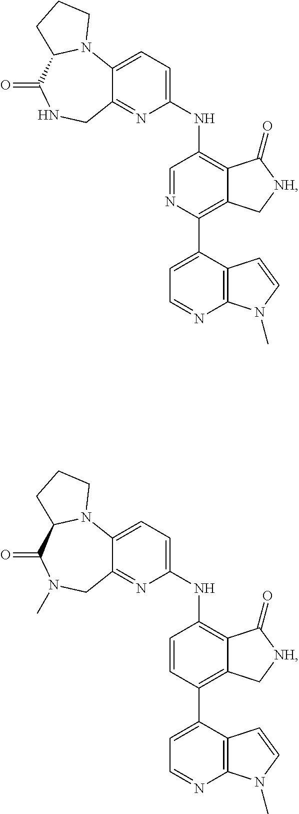 [SMILES: Cn1ccc2c(-c3ncc(Nc4ccc5c(n4)CNC(=O)[C@@H]4CCCN54)c4c3CNC4=O)ccnc21 ; CN1Cc2nc(Nc3ccc(-c4ccnc5c4ccn5C)c4c3C(=O)NC4)ccc2N2CCC[C@@H]2C1=O]

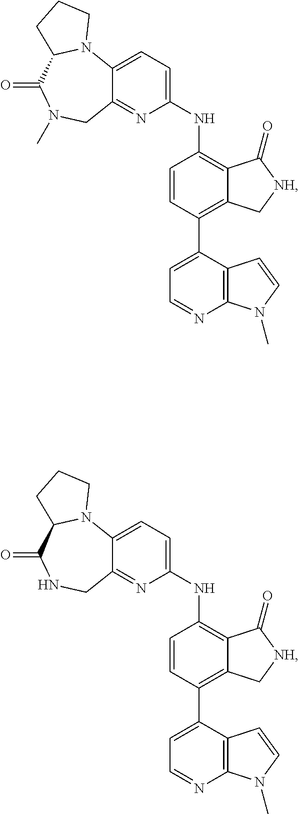 [SMILES: CN1Cc2nc(Nc3ccc(-c4ccnc5c4ccn5C)c4c3C(=O)NC4)ccc2N2CCC[C@H]2C1=O ; Cn1ccc2c(-c3ccc(Nc4ccc5c(n4)CNC(=O)[C@H]4CCCN54)c4c3CNC4=O)ccnc21]

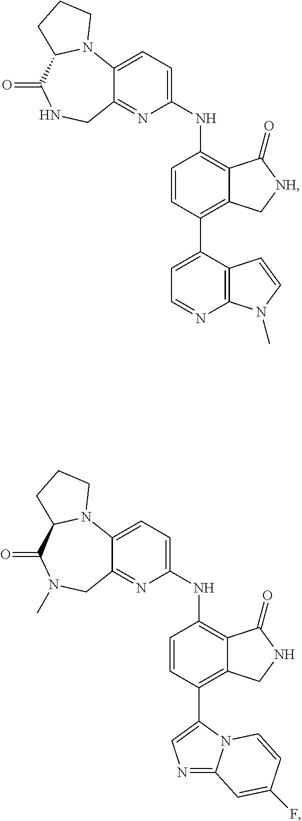 [SMILES: Cn1ccc2c(-c3ccc(Nc4ccc5c(n4)CNC(=O)[C@@H]4CCCN54)c4c3CNC4=O)ccnc21 ; CN1Cc2nc(Nc3ccc(-c4cnc5cc(F)ccn45)c4c3C(=O)NC4)ccc2N2CCC[C@@H]2C1=O]

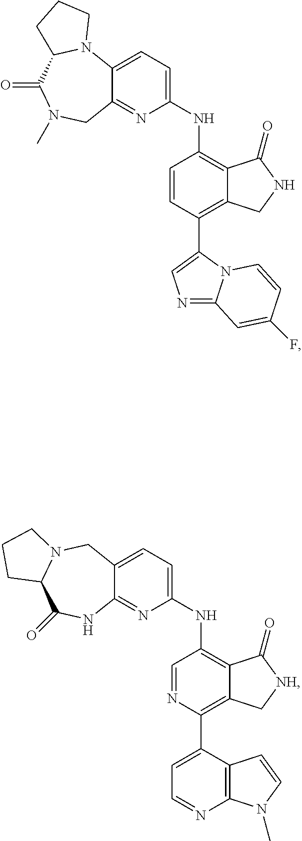 [SMILES: CN1Cc2nc(Nc3ccc(-c4cnc5cc(F)ccn45)c4c3C(=O)NC4)ccc2N2CCC[C@H]2C1=O ; Cn1ccc2c(-c3ncc(Nc4ccc5c(n4)NC(=O)[C@H]4CCCN4C5)c4c3CNC4=O)ccnc21]

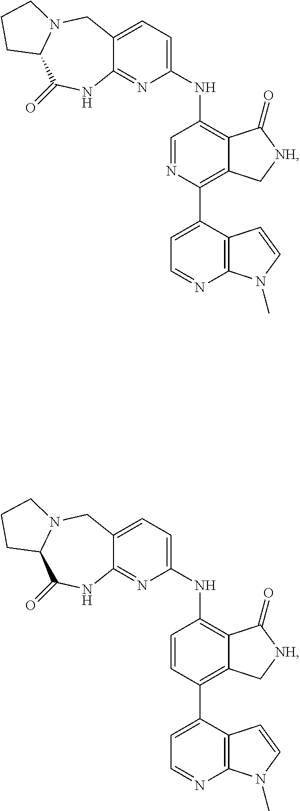 [SMILES: Cn1ccc2c(-c3ncc(Nc4ccc5c(n4)NC(=O)[C@@H]4CCCN4C5)c4c3CNC4=O)ccnc21 ; Cn1ccc2c(-c3ccc(Nc4ccc5c(n4)NC(=O)[C@H]4CCCN4C5)c4c3CNC4=O)ccnc21]

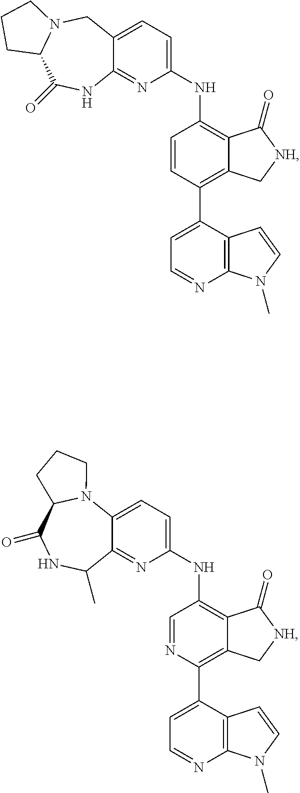

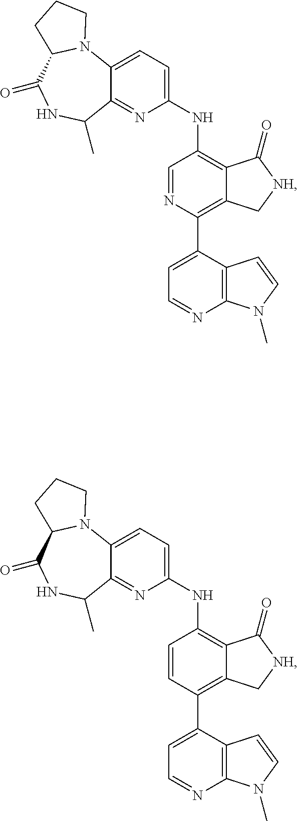

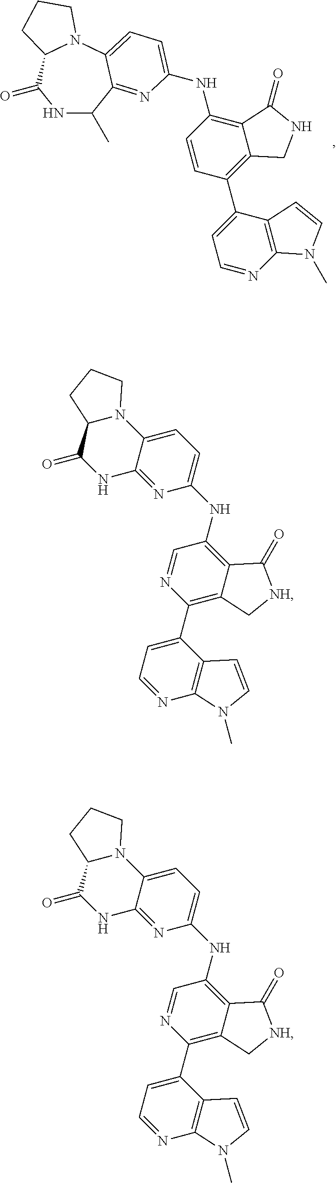

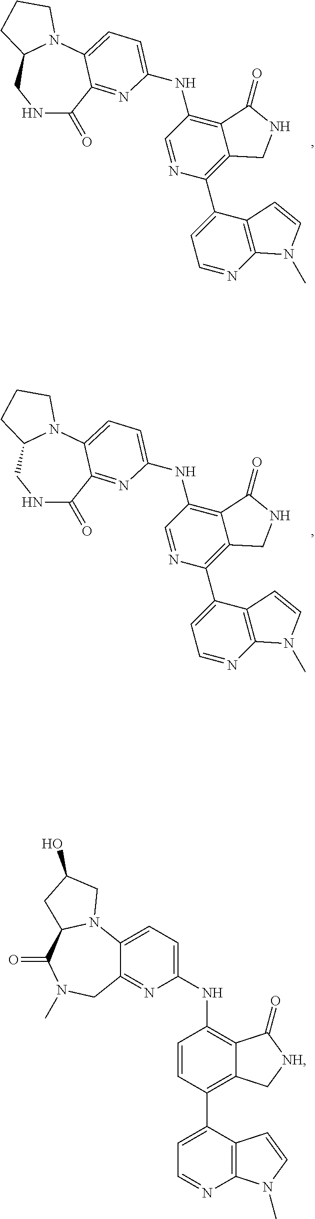 [SMILES: Cn1ccc2c(-c3ncc(Nc4ccc5c(n4)C(=O)NC[C@H]4CCCN54)c4c3CNC4=O)ccnc21 ; Cn1ccc2c(-c3ncc(Nc4ccc5c(n4)C(=O)NC[C@@H]4CCCN54)c4c3CNC4=O)ccnc21 ; CN1Cc2nc(Nc3ccc(-c4ccnc5c4ccn5C)c4c3C(=O)NC4)ccc2N2C[C@H](O)C[C@@H]2C1=O]

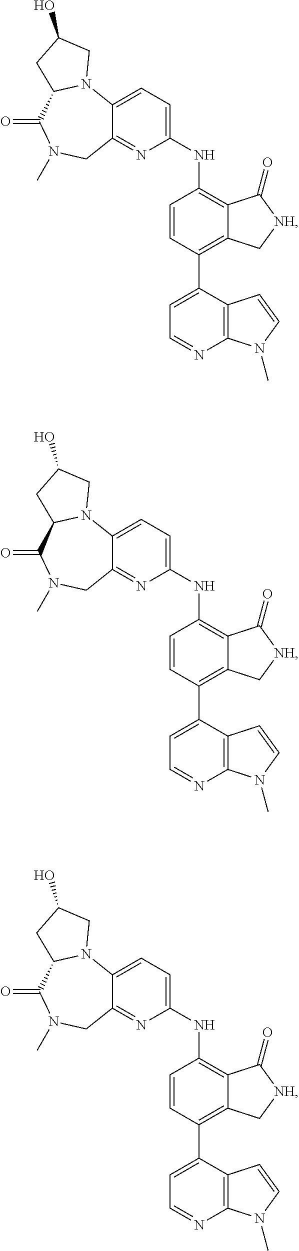 [SMILES: CN1Cc2nc(Nc3ccc(-c4ccnc5c4ccn5C)c4c3C(=O)NC4)ccc2N2C[C@H](O)C[C@H]2C1=O ; CN1Cc2nc(Nc3ccc(-c4ccnc5c4ccn5C)c4c3C(=O)NC4)ccc2N2C[C@@H](O)C[C@@H]2C1=O ; CN1Cc2nc(Nc3ccc(-c4ccnc5c4ccn5C)c4c3C(=O)NC4)ccc2N2C[C@@H](O)C[C@H]2C1=O]

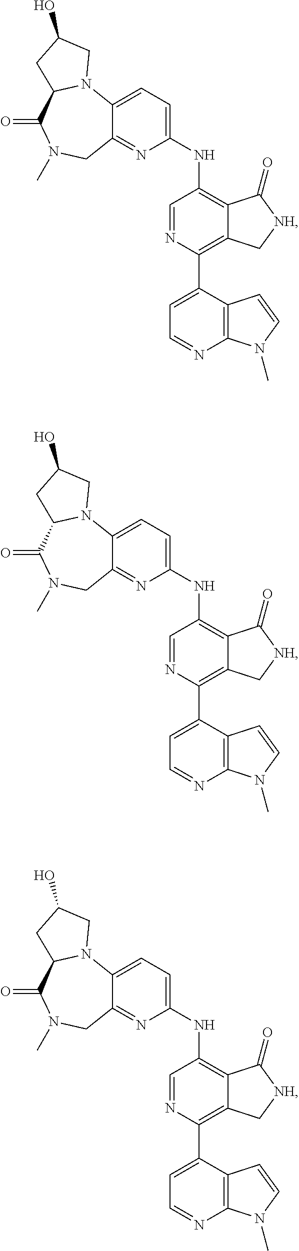 [SMILES: CN1Cc2nc(Nc3cnc(-c4ccnc5c4ccn5C)c4c3C(=O)NC4)ccc2N2C[C@H](O)C[C@@H]2C1=O ; CN1Cc2nc(Nc3cnc(-c4ccnc5c4ccn5C)c4c3C(=O)NC4)ccc2N2C[C@H](O)C[C@H]2C1=O ; CN1Cc2nc(Nc3cnc(-c4ccnc5c4ccn5C)c4c3C(=O)NC4)ccc2N2C[C@@H](O)C[C@@H]2C1=O]

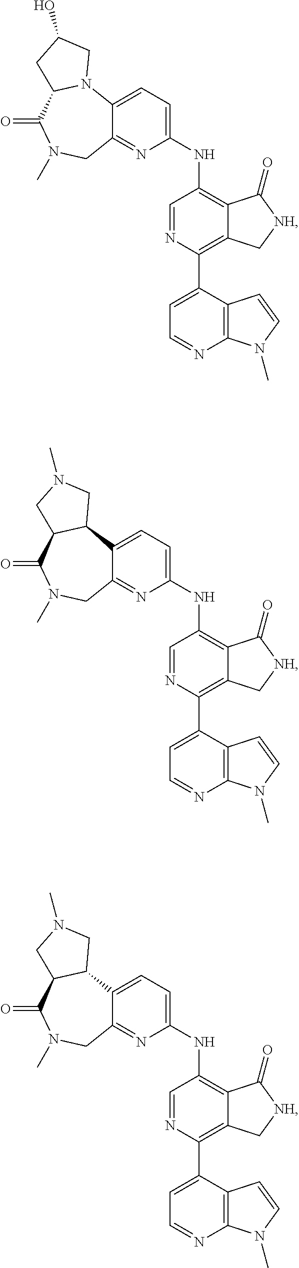 [SMILES: CN1Cc2nc(Nc3cnc(-c4ccnc5c4ccn5C)c4c3C(=O)NC4)ccc2N2C[C@@H](O)C[C@H]2C1=O ; CN1C[C@@H]2C(=O)N(C)Cc3nc(Nc4cnc(-c5ccnc6c5ccn6C)c5c4C(=O)NC5)ccc3[C@@H]2C1 ; CN1C[C@@H]2C(=O)N(C)Cc3nc(Nc4cnc(-c5ccnc6c5ccn6C)c5c4C(=O)NC5)ccc3[C@H]2C1]

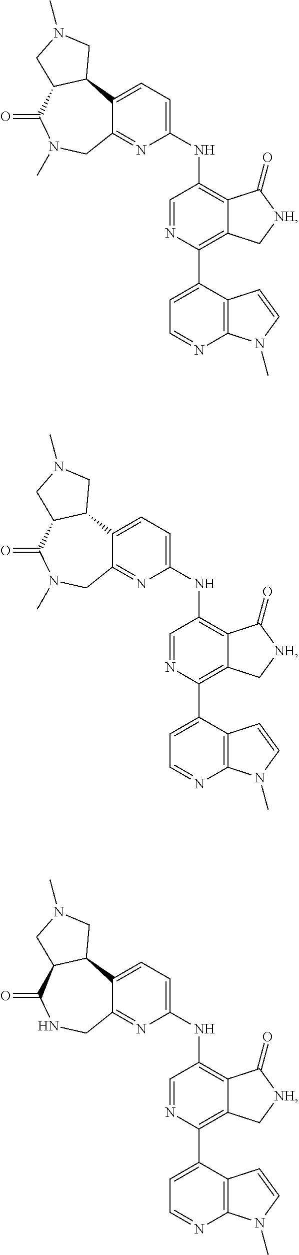 [SMILES: CN1C[C@H]2C(=O)N(C)Cc3nc(Nc4cnc(-c5ccnc6c5ccn6C)c5c4C(=O)NC5)ccc3[C@@H]2C1 ; CN1C[C@@H]2c3ccc(Nc4cnc(-c5ccnc6c5ccn6C)c5c4C(=O)NC5)nc3CN(C)C(=O)[C@@H]2C1 ; CN1C[C@@H]2C(=O)NCc3nc(Nc4cnc(-c5ccnc6c5ccn6C)c5c4C(=O)NC5)ccc3[C@@H]2C1]

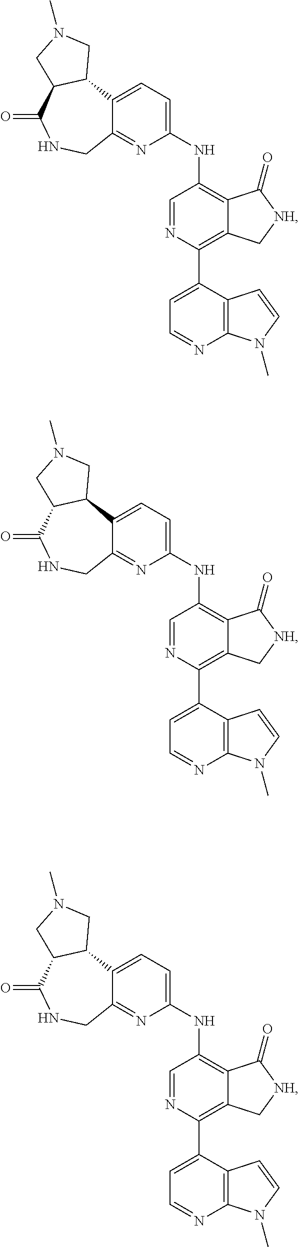 [SMILES: CN1C[C@@H]2C(=O)NCc3nc(Nc4cnc(-c5ccnc6c5ccn6C)c5c4C(=O)NC5)ccc3[C@H]2C1 ; CN1C[C@H]2C(=O)NCc3nc(Nc4cnc(-c5ccnc6c5ccn6C)c5c4C(=O)NC5)ccc3[C@@H]2C1 ; CN1C[C@@H]2c3ccc(Nc4cnc(-c5ccnc6c5ccn6C)c5c4C(=O)NC5)nc3CNC(=O)[C@@H]2C1]

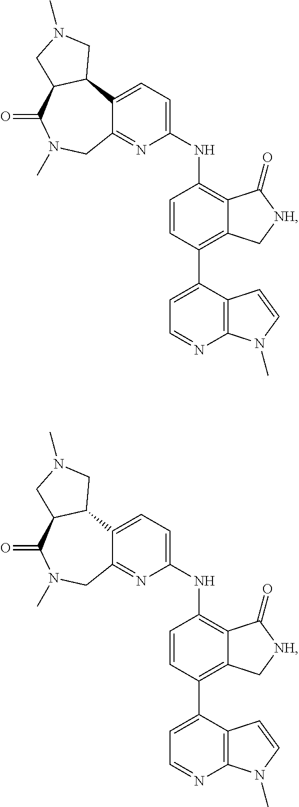 [SMILES: CN1C[C@@H]2C(=O)N(C)Cc3nc(Nc4ccc(-c5ccnc6c5ccn6C)c5c4C(=O)NC5)ccc3[C@@H]2C1 ; CN1C[C@@H]2C(=O)N(C)Cc3nc(Nc4ccc(-c5ccnc6c5ccn6C)c5c4C(=O)NC5)ccc3[C@H]2C1]

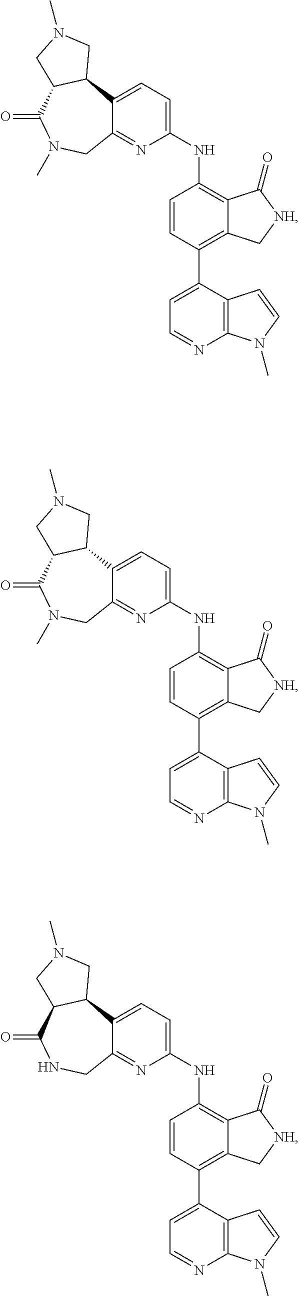 [SMILES: CN1C[C@H]2C(=O)N(C)Cc3nc(Nc4ccc(-c5ccnc6c5ccn6C)c5c4C(=O)NC5)ccc3[C@@H]2C1 ; CN1C[C@@H]2c3ccc(Nc4ccc(-c5ccnc6c5ccn6C)c5c4C(=O)NC5)nc3CN(C)C(=O)[C@@H]2C1 ; CN1C[C@@H]2C(=O)NCc3nc(Nc4ccc(-c5ccnc6c5ccn6C)c5c4C(=O)NC5)ccc3[C@@H]2C1]

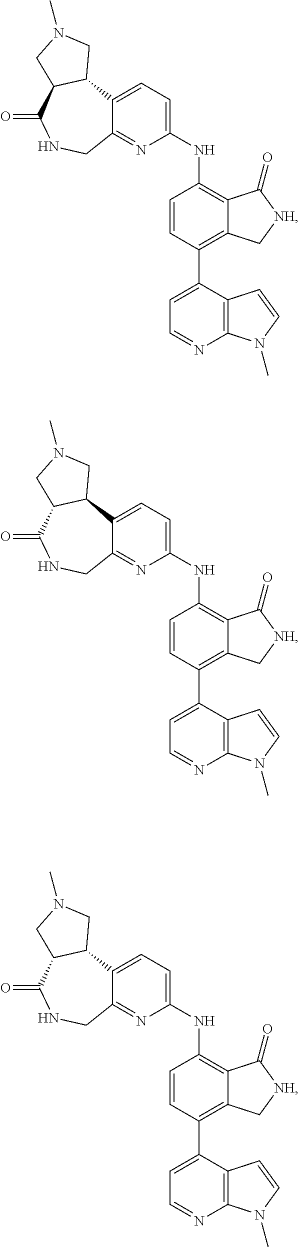 [SMILES: CN1C[C@@H]2C(=O)NCc3nc(Nc4ccc(-c5ccnc6c5ccn6C)c5c4C(=O)NC5)ccc3[C@H]2C1 ; CN1C[C@H]2C(=O)NCc3nc(Nc4ccc(-c5ccnc6c5ccn6C)c5c4C(=O)NC5)ccc3[C@@H]2C1 ; CN1C[C@@H]2c3ccc(Nc4ccc(-c5ccnc6c5ccn6C)c5c4C(=O)NC5)nc3CNC(=O)[C@@H]2C1]

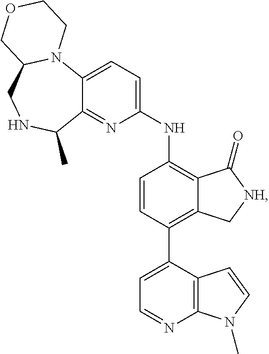 [SMILES: C[C@H]1NC[C@H]2COCCN2c2ccc(Nc3ccc(-c4ccnc5c4ccn5C)c4c3C(=O)NC4)nc21]

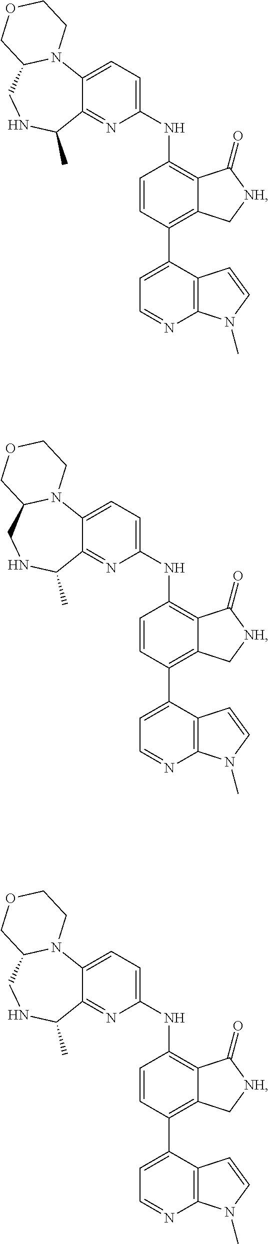 [SMILES: C[C@H]1NC[C@@H]2COCCN2c2ccc(Nc3ccc(-c4ccnc5c4ccn5C)c4c3C(=O)NC4)nc21 ; C[C@@H]1NC[C@H]2COCCN2c2ccc(Nc3ccc(-c4ccnc5c4ccn5C)c4c3C(=O)NC4)nc21 ; C[C@@H]1NC[C@@H]2COCCN2c2ccc(Nc3ccc(-c4ccnc5c4ccn5C)c4c3C(=O)NC4)nc21]

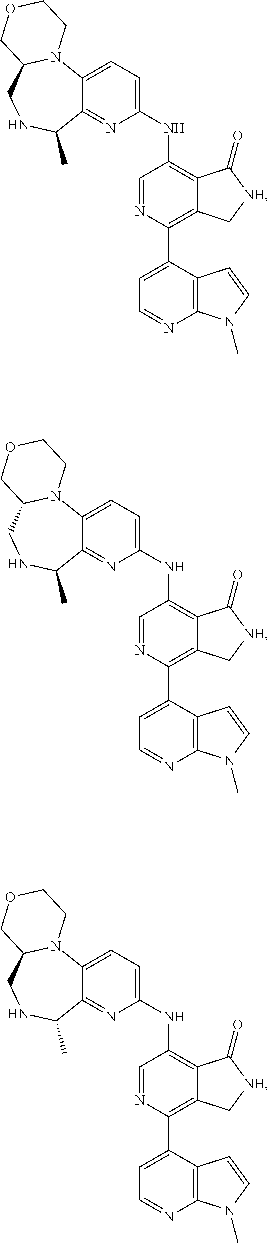 [SMILES: C[C@H]1NC[C@H]2COCCN2c2ccc(Nc3cnc(-c4ccnc5c4ccn5C)c4c3C(=O)NC4)nc21 ; C[C@H]1NC[C@@H]2COCCN2c2ccc(Nc3cnc(-c4ccnc5c4ccn5C)c4c3C(=O)NC4)nc21 ; C[C@@H]1NC[C@H]2COCCN2c2ccc(Nc3cnc(-c4ccnc5c4ccn5C)c4c3C(=O)NC4)nc21]

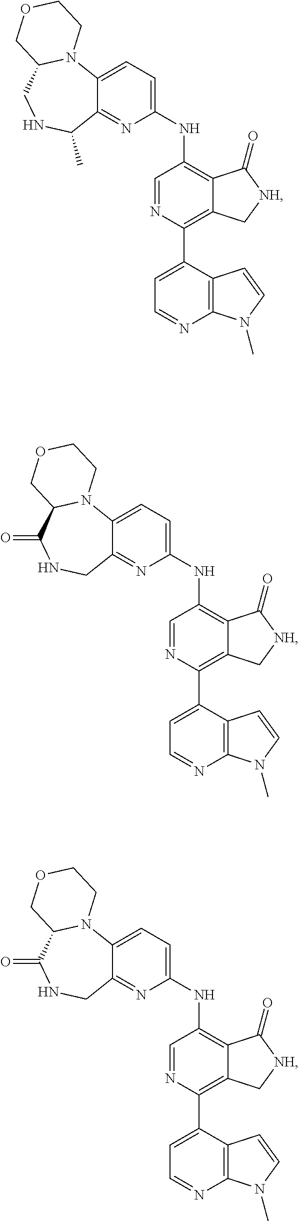 [SMILES: C[C@@H]1NC[C@@H]2COCCN2c2ccc(Nc3cnc(-c4ccnc5c4ccn5C)c4c3C(=O)NC4)nc21 ; Cn1ccc2c(-c3ncc(Nc4ccc5c(n4)CNC(=O)[C@H]4COCCN54)c4c3CNC4=O)ccnc21 ; Cn1ccc2c(-c3ncc(Nc4ccc5c(n4)CNC(=O)[C@@H]4COCCN54)c4c3CNC4=O)ccnc21]

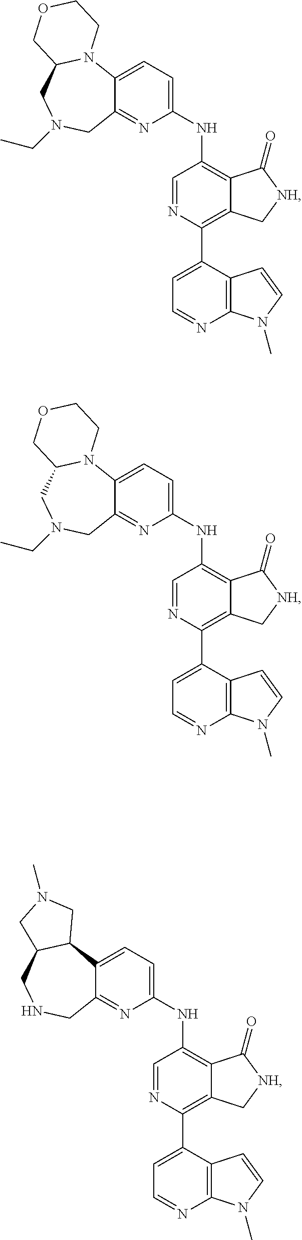 [SMILES: CCN1Cc2nc(Nc3cnc(-c4ccnc5c4ccn5C)c4c3C(=O)NC4)ccc2N2CCOC[C@@H]2C1 ; CCN1Cc2nc(Nc3cnc(-c4ccnc5c4ccn5C)c4c3C(=O)NC4)ccc2N2CCOC[C@H]2C1 ; CN1C[C@@H]2CNCc3nc(Nc4cnc(-c5ccnc6c5ccn6C)c5c4C(=O)NC5)ccc3[C@@H]2C1]

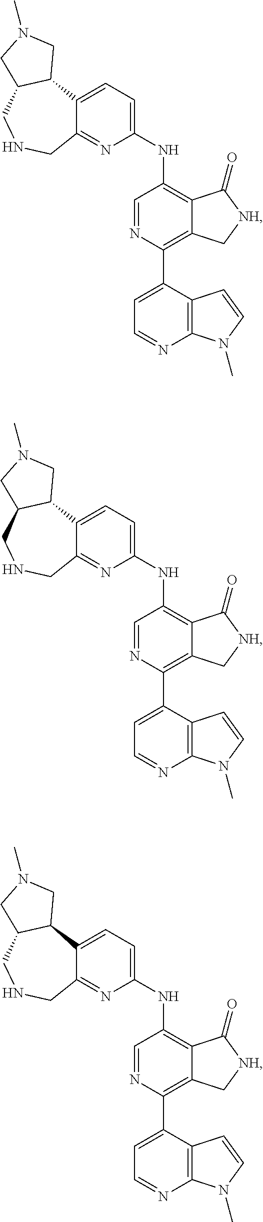 [SMILES: CN1C[C@H]2CNCc3nc(Nc4cnc(-c5ccnc6c5ccn6C)c5c4C(=O)NC5)ccc3[C@H]2C1 ; CN1C[C@@H]2CNCc3nc(Nc4cnc(-c5ccnc6c5ccn6C)c5c4C(=O)NC5)ccc3[C@H]2C1 ; CN1C[C@H]2CNCc3nc(Nc4cnc(-c5ccnc6c5ccn6C)c5c4C(=O)NC5)ccc3[C@@H]2C1]

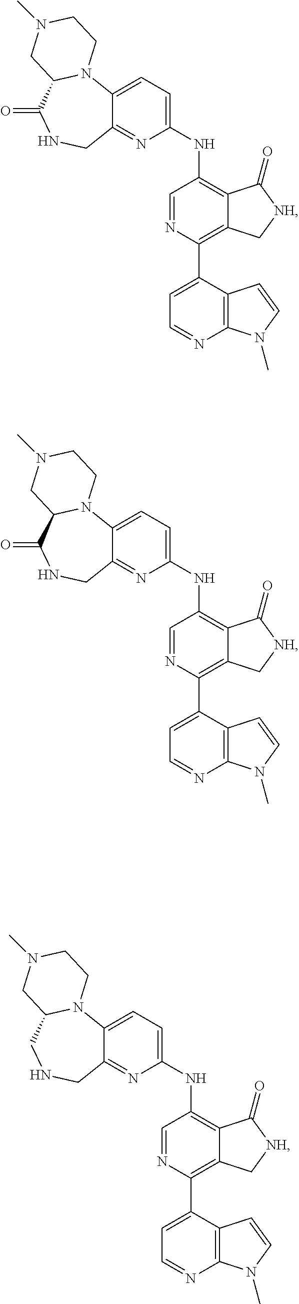 [SMILES: CN1CCN2c3ccc(Nc4cnc(-c5ccnc6c5ccn6C)c5c4C(=O)NC5)nc3CNC(=O)[C@@H]2C1 ; CN1CCN2c3ccc(Nc4cnc(-c5ccnc6c5ccn6C)c5c4C(=O)NC5)nc3CNC(=O)[C@H]2C1 ; CN1CCN2c3ccc(Nc4cnc(-c5ccnc6c5ccn6C)c5c4C(=O)NC5)nc3CNC[C@@H]2C1]

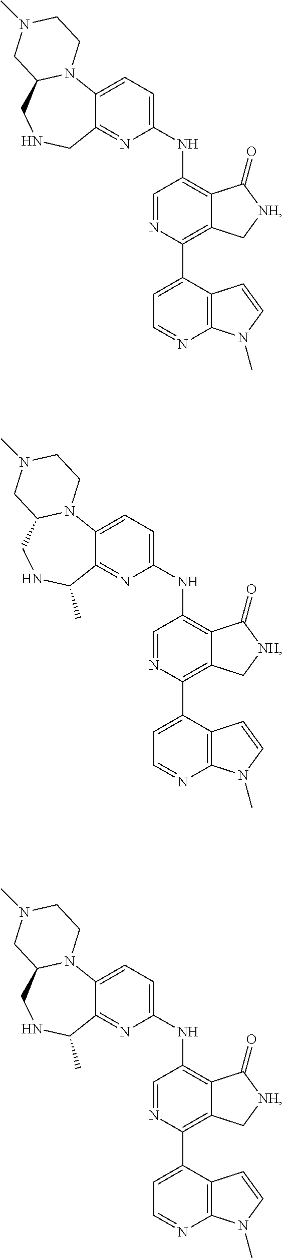 [SMILES: CN1CCN2c3ccc(Nc4cnc(-c5ccnc6c5ccn6C)c5c4C(=O)NC5)nc3CNC[C@H]2C1 ; C[C@@H]1NC[C@@H]2CN(C)CCN2c2ccc(Nc3cnc(-c4ccnc5c4ccn5C)c4c3C(=O)NC4)nc21 ; C[C@@H]1NC[C@H]2CN(C)CCN2c2ccc(Nc3cnc(-c4ccnc5c4ccn5C)c4c3C(=O)NC4)nc21]

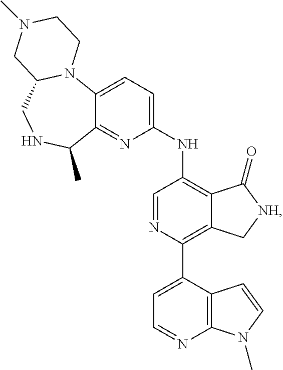 [SMILES: C[C@H]1NC[C@@H]2CN(C)CCN2c2ccc(Nc3cnc(-c4ccnc5c4ccn5C)c4c3C(=O)NC4)nc21]

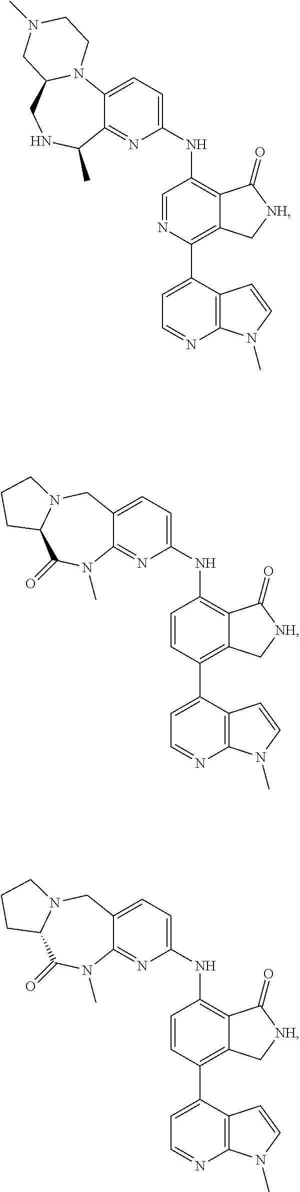 [SMILES: C[C@H]1NC[C@H]2CN(C)CCN2c2ccc(Nc3cnc(-c4ccnc5c4ccn5C)c4c3C(=O)NC4)nc21 ; CN1C(=O)[C@H]2CCCN2Cc2ccc(Nc3ccc(-c4ccnc5c4ccn5C)c4c3C(=O)NC4)nc21 ; CN1C(=O)[C@@H]2CCCN2Cc2ccc(Nc3ccc(-c4ccnc5c4ccn5C)c4c3C(=O)NC4)nc21]

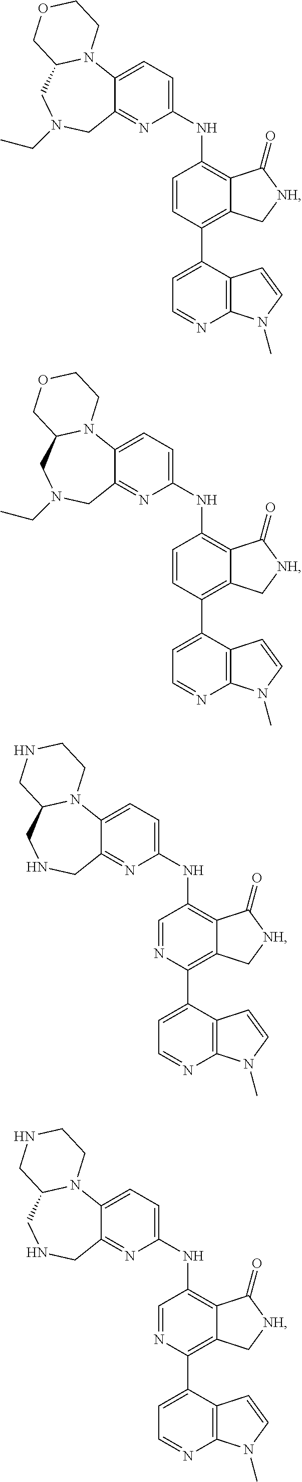 [SMILES: CCN1Cc2nc(Nc3ccc(-c4ccnc5c4ccn5C)c4c3C(=O)NC4)ccc2N2CCOC[C@H]2C1 ; CCN1Cc2nc(Nc3ccc(-c4ccnc5c4ccn5C)c4c3C(=O)NC4)ccc2N2CCOC[C@@H]2C1 ; Cn1ccc2c(-c3ncc(Nc4ccc5c(n4)CNC[C@H]4CNCCN54)c4c3CNC4=O)ccnc21 ; Cn1ccc2c(-c3ncc(Nc4ccc5c(n4)CNC[C@@H]4CNCCN54)c4c3CNC4=O)ccnc21]

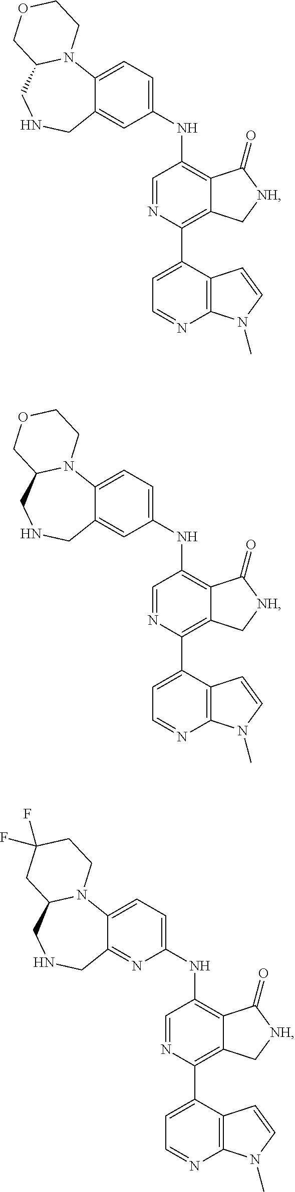 [SMILES: Cn1ccc2c(-c3ncc(Nc4ccc5c(c4)CNC[C@@H]4COCCN54)c4c3CNC4=O)ccnc21 ; Cn1ccc2c(-c3ncc(Nc4ccc5c(c4)CNC[C@H]4COCCN54)c4c3CNC4=O)ccnc21 ; Cn1ccc2c(-c3ncc(Nc4ccc5c(n4)CNC[C@H]4CC(F)(F)CCN54)c4c3CNC4=O)ccnc21]

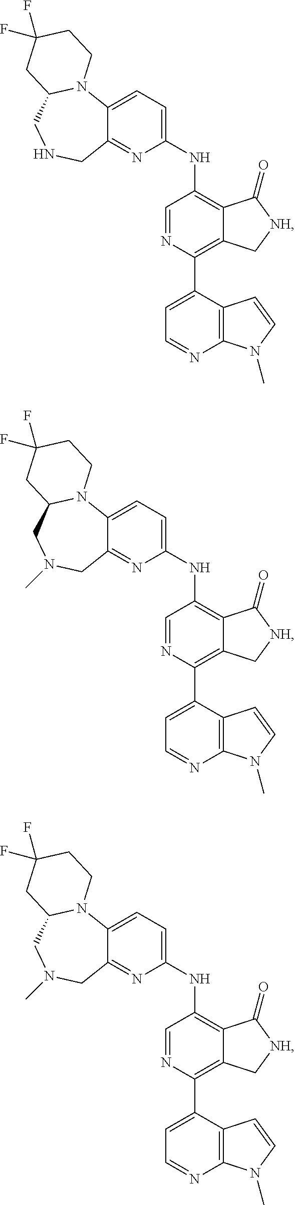 [SMILES: Cn1ccc2c(-c3ncc(Nc4ccc5c(n4)CNC[C@@H]4CC(F)(F)CCN54)c4c3CNC4=O)ccnc21 ; CN1Cc2nc(Nc3cnc(-c4ccnc5c4ccn5C)c4c3C(=O)NC4)ccc2N2CCC(F)(F)C[C@@H]2C1 ; CN1Cc2nc(Nc3cnc(-c4ccnc5c4ccn5C)c4c3C(=O)NC4)ccc2N2CCC(F)(F)C[C@H]2C1]

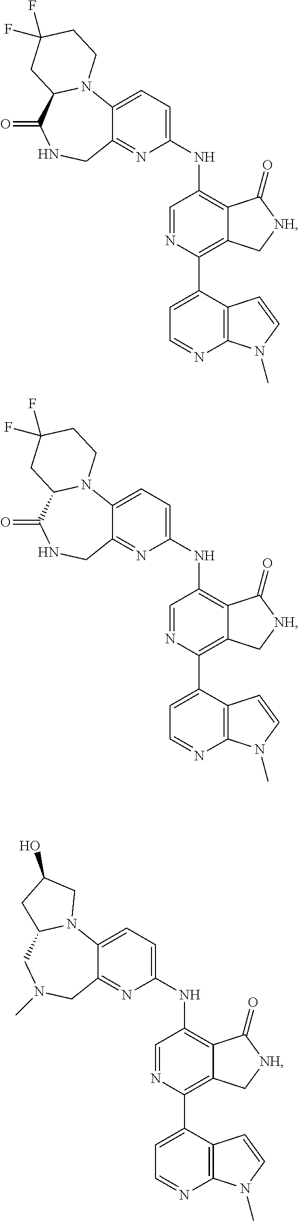 [SMILES: Cn1ccc2c(-c3ncc(Nc4ccc5c(n4)CNC(=O)[C@H]4CC(F)(F)CCN54)c4c3CNC4=O)ccnc21 ; Cn1ccc2c(-c3ncc(Nc4ccc5c(n4)CNC(=O)[C@@H]4CC(F)(F)CCN54)c4c3CNC4=O)ccnc21 ; CN1Cc2nc(Nc3cnc(-c4ccnc5c4ccn5C)c4c3C(=O)NC4)ccc2N2C[C@H](O)C[C@H]2C1]

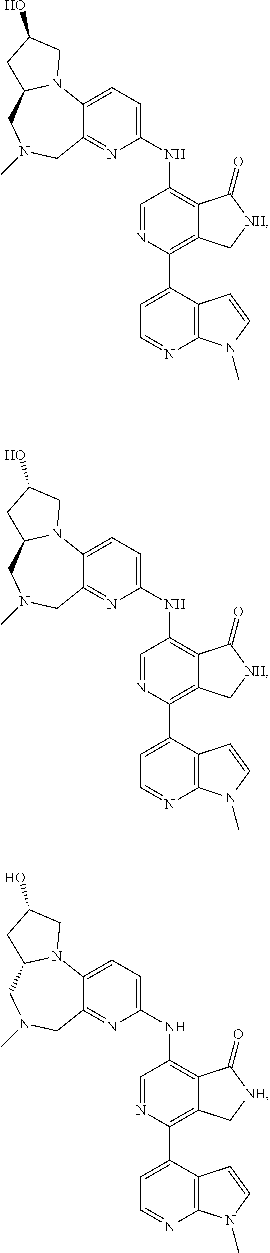 [SMILES: CN1Cc2nc(Nc3cnc(-c4ccnc5c4ccn5C)c4c3C(=O)NC4)ccc2N2C[C@H](O)C[C@@H]2C1 ; CN1Cc2nc(Nc3cnc(-c4ccnc5c4ccn5C)c4c3C(=O)NC4)ccc2N2C[C@@H](O)C[C@@H]2C1 ; CN1Cc2nc(Nc3cnc(-c4ccnc5c4ccn5C)c4c3C(=O)NC4)ccc2N2C[C@@H](O)C[C@H]2C1]

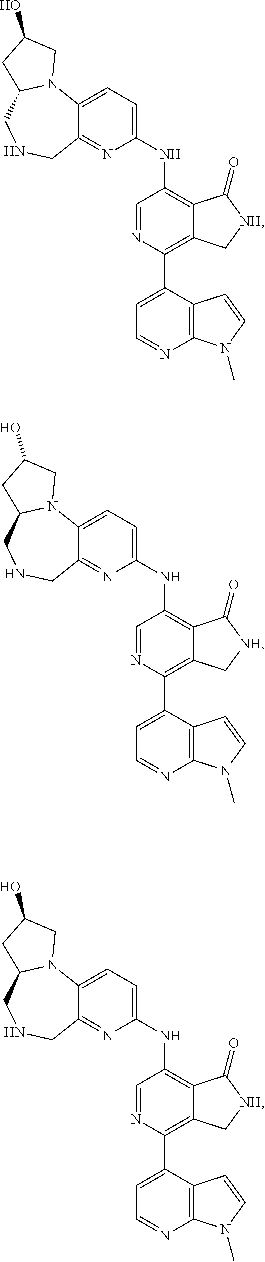 [SMILES: Cn1ccc2c(-c3ncc(Nc4ccc5c(n4)CNC[C@@H]4C[C@@H](O)CN54)c4c3CNC4=O)ccnc21 ; Cn1ccc2c(-c3ncc(Nc4ccc5c(n4)CNC[C@H]4C[C@H](O)CN54)c4c3CNC4=O)ccnc21 ; Cn1ccc2c(-c3ncc(Nc4ccc5c(n4)CNC[C@H]4C[C@@H](O)CN54)c4c3CNC4=O)ccnc21]

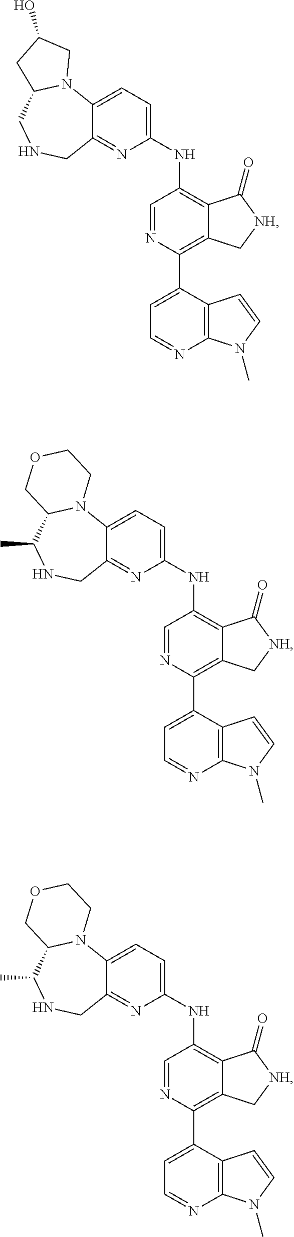 [SMILES: Cn1ccc2c(-c3ncc(Nc4ccc5c(n4)CNC[C@@H]4C[C@H](O)CN54)c4c3CNC4=O)ccnc21 ; C[C@@H]1NCc2nc(Nc3cnc(-c4ccnc5c4ccn5C)c4c3C(=O)NC4)ccc2N2CCOC[C@@H]12 ; C[C@H]1NCc2nc(Nc3cnc(-c4ccnc5c4ccn5C)c4c3C(=O)NC4)ccc2N2CCOC[C@@H]12]

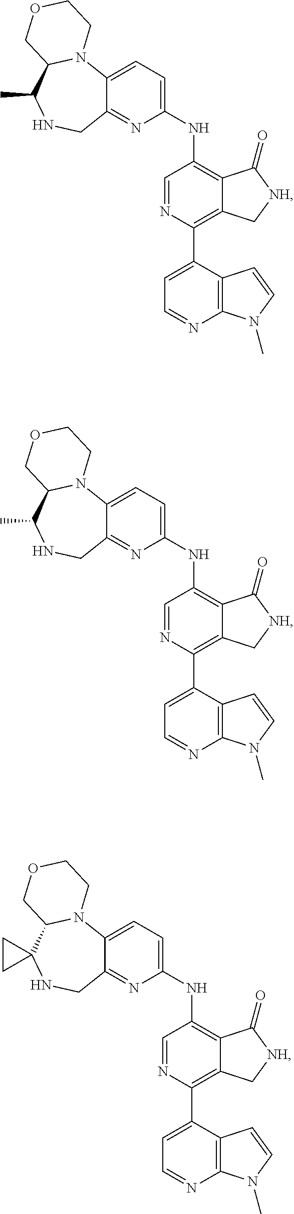 [SMILES: C[C@@H]1NCc2nc(Nc3cnc(-c4ccnc5c4ccn5C)c4c3C(=O)NC4)ccc2N2CCOC[C@H]12 ; C[C@H]1NCc2nc(Nc3cnc(-c4ccnc5c4ccn5C)c4c3C(=O)NC4)ccc2N2CCOC[C@H]12 ; Cn1ccc2c(-c3ncc(Nc4ccc5c(n4)CNC4(CC4)[C@@H]4COCCN54)c4c3CNC4=O)ccnc21]

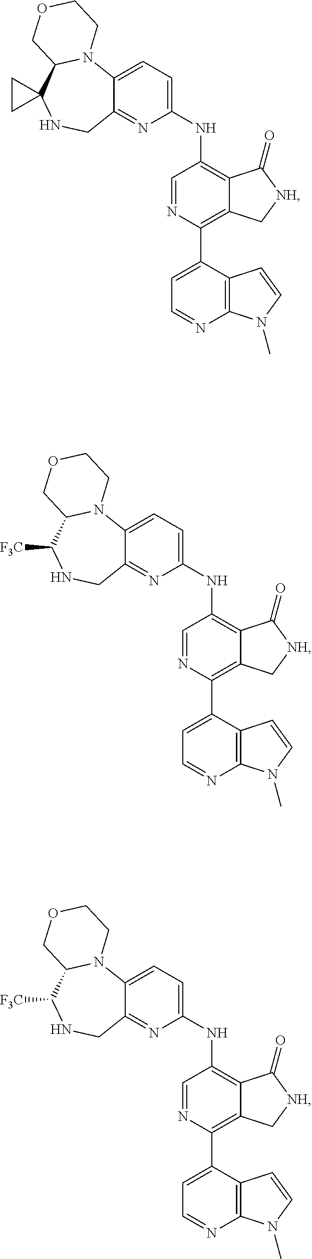 [SMILES: Cn1ccc2c(-c3ncc(Nc4ccc5c(n4)CNC4(CC4)[C@H]4COCCN54)c4c3CNC4=O)ccnc21 ; Cn1ccc2c(-c3ncc(Nc4ccc5c(n4)CN[C@@H](C(F)(F)F)[C@@H]4COCCN54)c4c3CNC4=O)ccnc21 ; Cn1ccc2c(-c3ncc(Nc4ccc5c(n4)CN[C@H](C(F)(F)F)[C@@H]4COCCN54)c4c3CNC4=O)ccnc21]

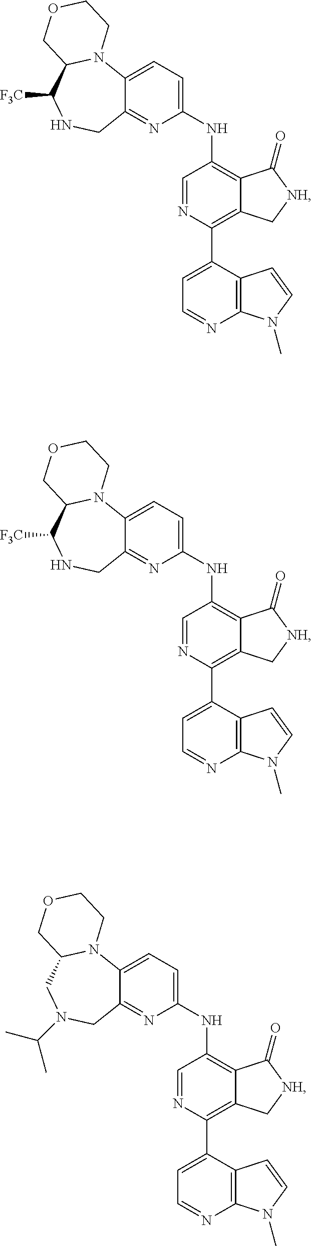 [SMILES: Cn1ccc2c(-c3ncc(Nc4ccc5c(n4)CN[C@@H](C(F)(F)F)[C@H]4COCCN54)c4c3CNC4=O)ccnc21 ; Cn1ccc2c(-c3ncc(Nc4ccc5c(n4)CN[C@H](C(F)(F)F)[C@H]4COCCN54)c4c3CNC4=O)ccnc21 ; CC(C)N1Cc2nc(Nc3cnc(-c4ccnc5c4ccn5C)c4c3C(=O)NC4)ccc2N2CCOC[C@H]2C1]

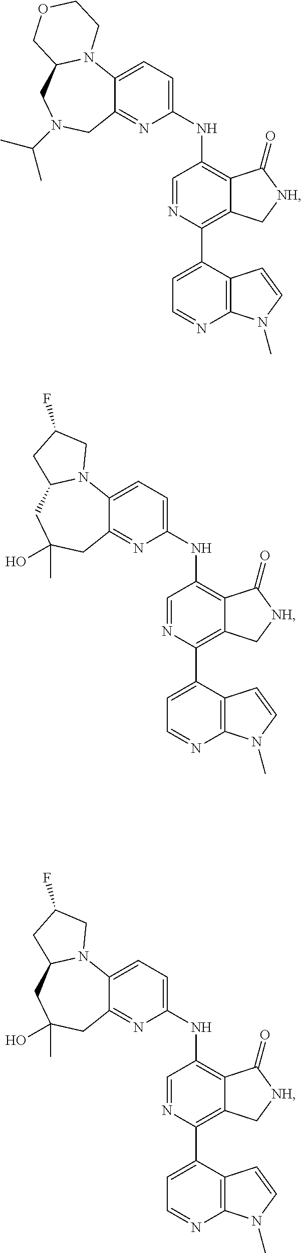

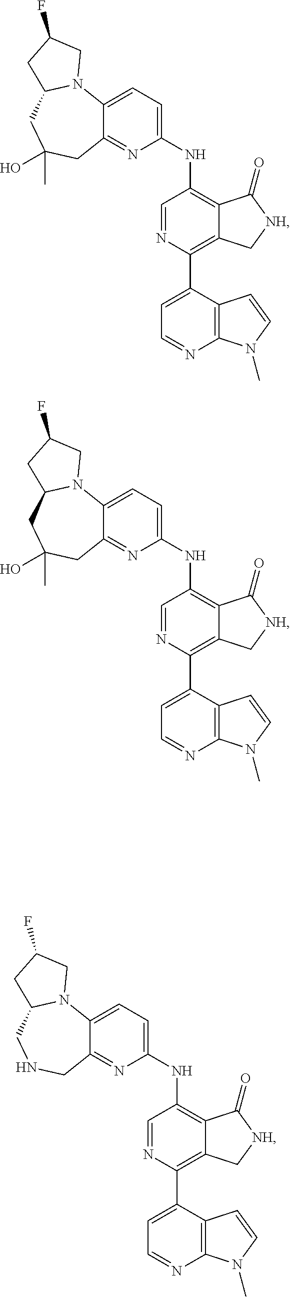

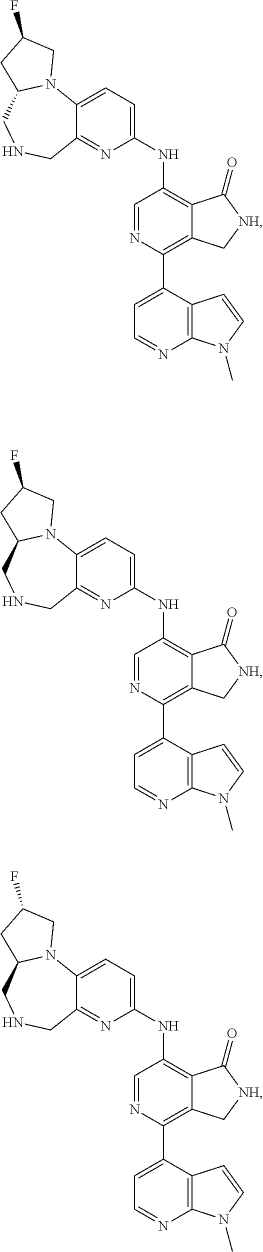 [SMILES: Cn1ccc2c(-c3ncc(Nc4ccc5c(n4)CNC[C@@H]4C[C@@H](F)CN54)c4c3CNC4=O)ccnc21 ; Cn1ccc2c(-c3ncc(Nc4ccc5c(n4)CNC[C@H]4C[C@@H](F)CN54)c4c3CNC4=O)ccnc21 ; Cn1ccc2c(-c3ncc(Nc4ccc5c(n4)CNC[C@H]4C[C@H](F)CN54)c4c3CNC4=O)ccnc21]

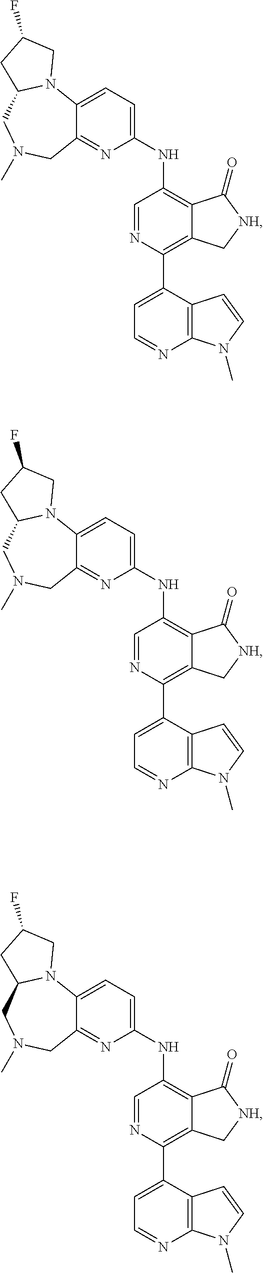 [SMILES: CN1Cc2nc(Nc3cnc(-c4ccnc5c4ccn5C)c4c3C(=O)NC4)ccc2N2C[C@@H](F)C[C@H]2C1 ; CN1Cc2nc(Nc3cnc(-c4ccnc5c4ccn5C)c4c3C(=O)NC4)ccc2N2C[C@H](F)C[C@H]2C1 ; CN1Cc2nc(Nc3cnc(-c4ccnc5c4ccn5C)c4c3C(=O)NC4)ccc2N2C[C@@H](F)C[C@@H]2C1]

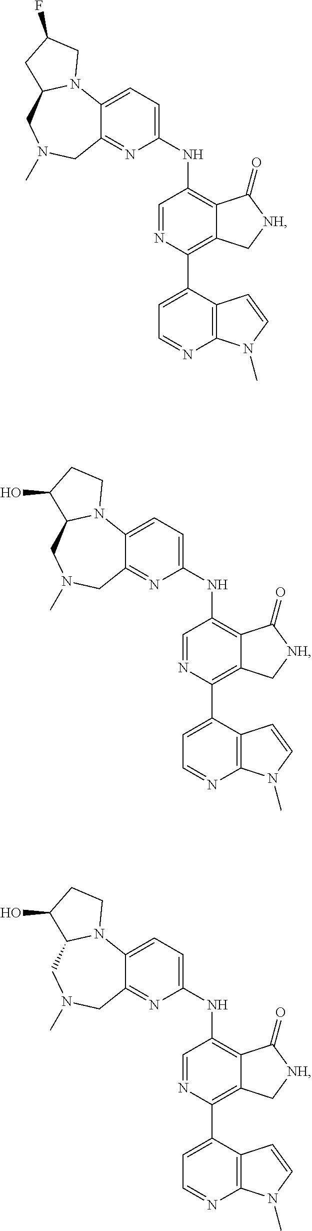 [SMILES: CN1Cc2nc(Nc3cnc(-c4ccnc5c4ccn5C)c4c3C(=O)NC4)ccc2N2C[C@H](F)C[C@@H]2C1 ; CN1Cc2nc(Nc3cnc(-c4ccnc5c4ccn5C)c4c3C(=O)NC4)ccc2N2CC[C@H](O)[C@@H]2C1 ; CN1Cc2nc(Nc3cnc(-c4ccnc5c4ccn5C)c4c3C(=O)NC4)ccc2N2CC[C@H](O)[C@H]2C1]

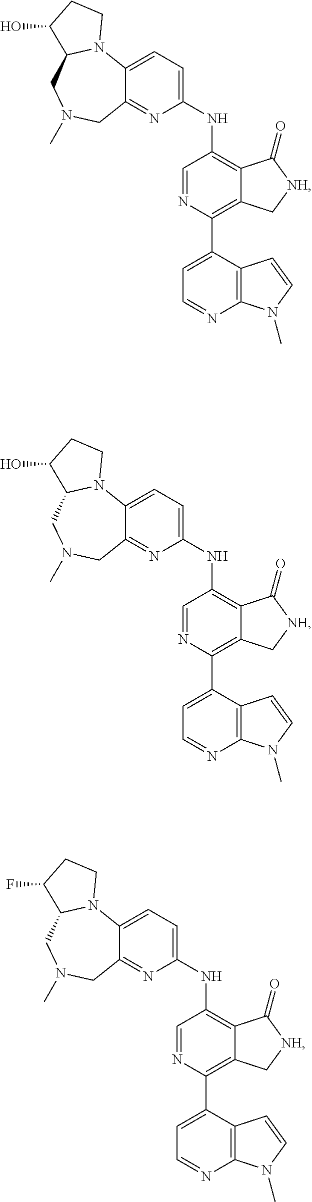 [SMILES: CN1Cc2nc(Nc3cnc(-c4ccnc5c4ccn5C)c4c3C(=O)NC4)ccc2N2CC[C@@H](O)[C@@H]2C1 ; CN1Cc2nc(Nc3cnc(-c4ccnc5c4ccn5C)c4c3C(=O)NC4)ccc2N2CC[C@@H](O)[C@H]2C1 ; CN1Cc2nc(Nc3cnc(-c4ccnc5c4ccn5C)c4c3C(=O)NC4)ccc2N2CC[C@@H](F)[C@H]2C1]

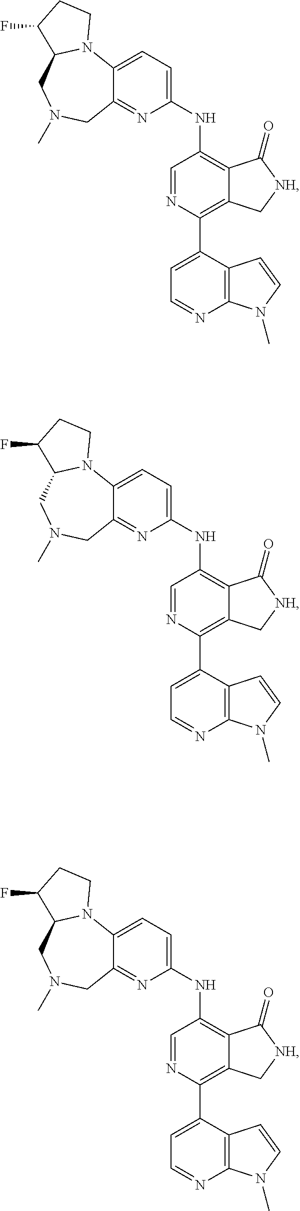 [SMILES: CN1Cc2nc(Nc3cnc(-c4ccnc5c4ccn5C)c4c3C(=O)NC4)ccc2N2CC[C@@H](F)[C@@H]2C1 ; CN1Cc2nc(Nc3cnc(-c4ccnc5c4ccn5C)c4c3C(=O)NC4)ccc2N2CC[C@H](F)[C@H]2C1 ; CN1Cc2nc(Nc3cnc(-c4ccnc5c4ccn5C)c4c3C(=O)NC4)ccc2N2CC[C@H](F)[C@@H]2C1]

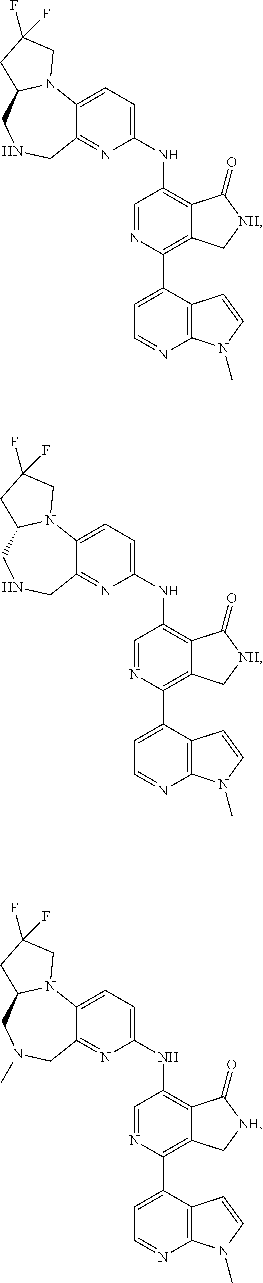 [SMILES: Cn1ccc2c(-c3ncc(Nc4ccc5c(n4)CNC[C@H]4CC(F)(F)CN54)c4c3CNC4=O)ccnc21 ; Cn1ccc2c(-c3ncc(Nc4ccc5c(n4)CNC[C@@H]4CC(F)(F)CN54)c4c3CNC4=O)ccnc21 ; CN1Cc2nc(Nc3cnc(-c4ccnc5c4ccn5C)c4c3C(=O)NC4)ccc2N2CC(F)(F)C[C@@H]2C1]

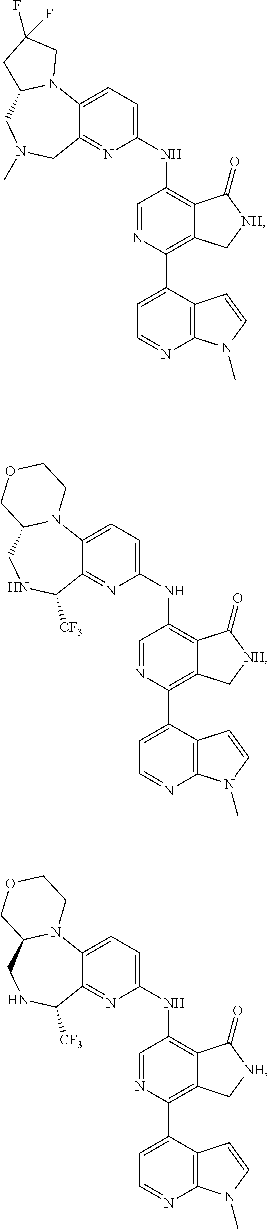 [SMILES: CN1Cc2nc(Nc3cnc(-c4ccnc5c4ccn5C)c4c3C(=O)NC4)ccc2N2CC(F)(F)C[C@H]2C1 ; Cn1ccc2c(-c3ncc(Nc4ccc5c(n4)[C@H](C(F)(F)F)NC[C@@H]4COCCN54)c4c3CNC4=O)ccnc21 ; Cn1ccc2c(-c3ncc(Nc4ccc5c(n4)[C@H](C(F)(F)F)NC[C@H]4COCCN54)c4c3CNC4=O)ccnc21]

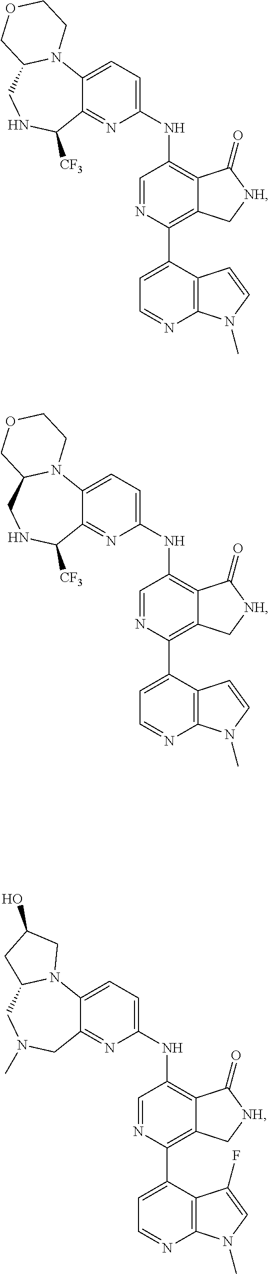 [SMILES: Cn1ccc2c(-c3ncc(Nc4ccc5c(n4)[C@@H](C(F)(F)F)NC[C@@H]4COCCN54)c4c3CNC4=O)ccnc21 ; Cn1ccc2c(-c3ncc(Nc4ccc5c(n4)[C@@H](C(F)(F)F)NC[C@H]4COCCN54)c4c3CNC4=O)ccnc21 ; CN1Cc2nc(Nc3cnc(-c4ccnc5c4c(F)cn5C)c4c3C(=O)NC4)ccc2N2C[C@H](O)C[C@H]2C1]

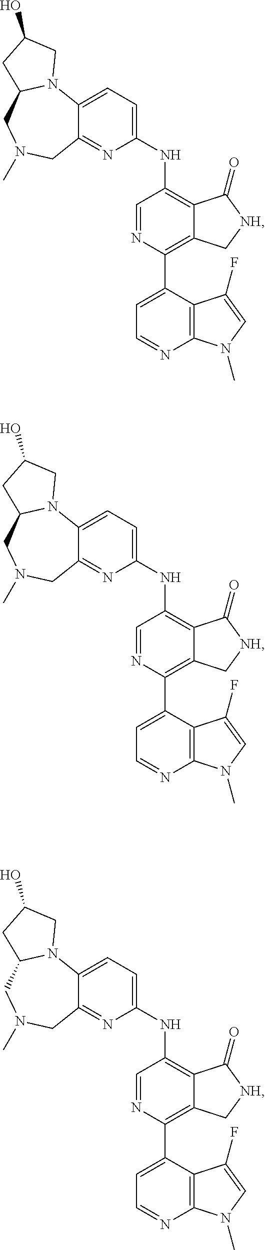 [SMILES: CN1Cc2nc(Nc3cnc(-c4ccnc5c4c(F)cn5C)c4c3C(=O)NC4)ccc2N2C[C@H](O)C[C@@H]2C1 ; CN1Cc2nc(Nc3cnc(-c4ccnc5c4c(F)cn5C)c4c3C(=O)NC4)ccc2N2C[C@@H](O)C[C@@H]2C1 ; CN1Cc2nc(Nc3cnc(-c4ccnc5c4c(F)cn5C)c4c3C(=O)NC4)ccc2N2C[C@@H](O)C[C@H]2C1]

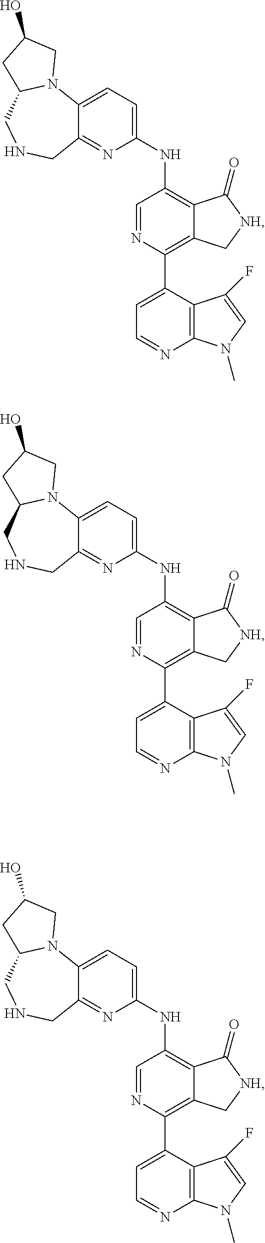 [SMILES: Cn1cc(F)c2c(-c3ncc(Nc4ccc5c(n4)CNC[C@@H]4C[C@@H](O)CN54)c4c3CNC4=O)ccnc21 ; Cn1cc(F)c2c(-c3ncc(Nc4ccc5c(n4)CNC[C@H]4C[C@@H](O)CN54)c4c3CNC4=O)ccnc21 ; Cn1cc(F)c2c(-c3ncc(Nc4ccc5c(n4)CNC[C@@H]4C[C@H](O)CN54)c4c3CNC4=O)ccnc21]

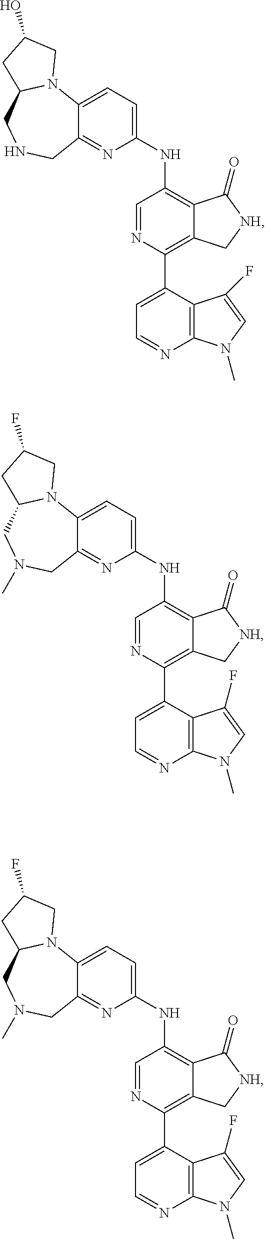 [SMILES: Cn1cc(F)c2c(-c3ncc(Nc4ccc5c(n4)CNC[C@H]4C[C@H](O)CN54)c4c3CNC4=O)ccnc21 ; CN1Cc2nc(Nc3cnc(-c4ccnc5c4c(F)cn5C)c4c3C(=O)NC4)ccc2N2C[C@@H](F)C[C@H]2C1 ; CN1Cc2nc(Nc3cnc(-c4ccnc5c4c(F)cn5C)c4c3C(=O)NC4)ccc2N2C[C@@H](F)C[C@@H]2C1]

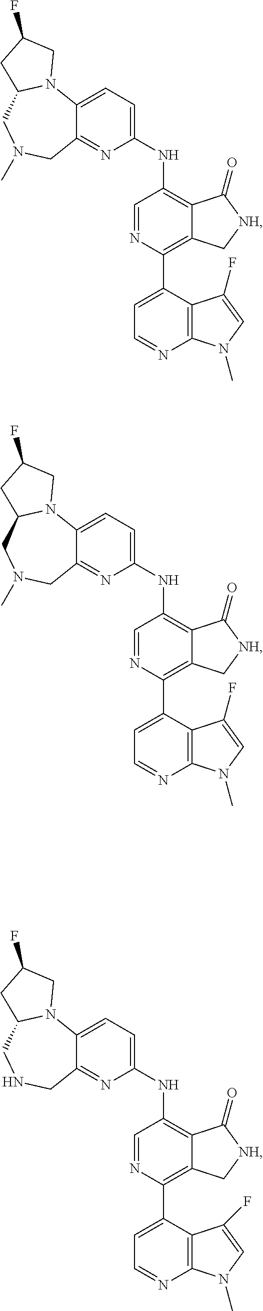 [SMILES: CN1Cc2nc(Nc3cnc(-c4ccnc5c4c(F)cn5C)c4c3C(=O)NC4)ccc2N2C[C@H](F)C[C@H]2C1 ; CN1Cc2nc(Nc3cnc(-c4ccnc5c4c(F)cn5C)c4c3C(=O)NC4)ccc2N2C[C@H](F)C[C@@H]2C1 ; Cn1cc(F)c2c(-c3ncc(Nc4ccc5c(n4)CNC[C@@H]4C[C@@H](F)CN54)c4c3CNC4=O)ccnc21]

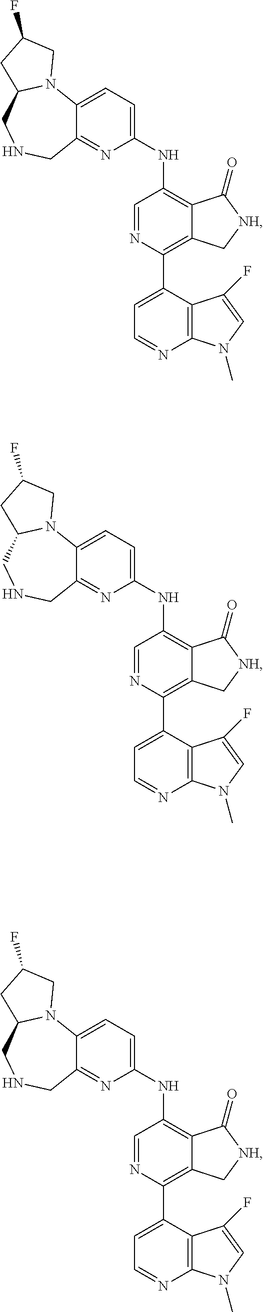 [SMILES: Cn1cc(F)c2c(-c3ncc(Nc4ccc5c(n4)CNC[C@H]4C[C@@H](F)CN54)c4c3CNC4=O)ccnc21 ; Cn1cc(F)c2c(-c3ncc(Nc4ccc5c(n4)CNC[C@@H]4C[C@H](F)CN54)c4c3CNC4=O)ccnc21 ; Cn1cc(F)c2c(-c3ncc(Nc4ccc5c(n4)CNC[C@H]4C[C@H](F)CN54)c4c3CNC4=O)ccnc21]

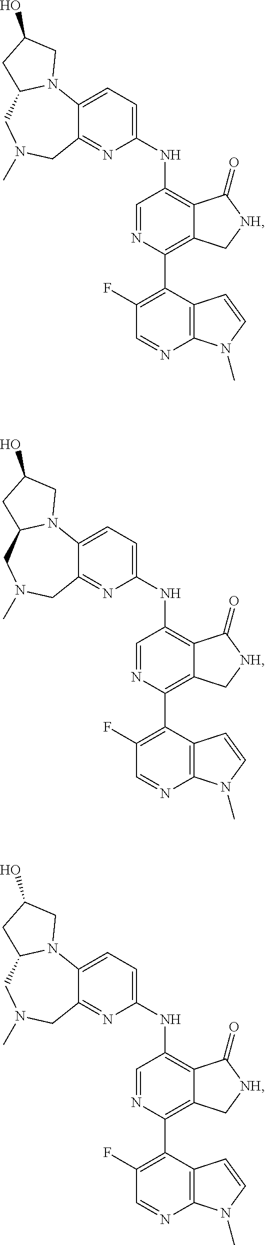 [SMILES: CN1Cc2nc(Nc3cnc(-c4c(F)cnc5c4ccn5C)c4c3C(=O)NC4)ccc2N2C[C@H](O)C[C@H]2C1 ; CN1Cc2nc(Nc3cnc(-c4c(F)cnc5c4ccn5C)c4c3C(=O)NC4)ccc2N2C[C@H](O)C[C@@H]2C1 ; CN1Cc2nc(Nc3cnc(-c4c(F)cnc5c4ccn5C)c4c3C(=O)NC4)ccc2N2C[C@@H](O)C[C@H]2C1]

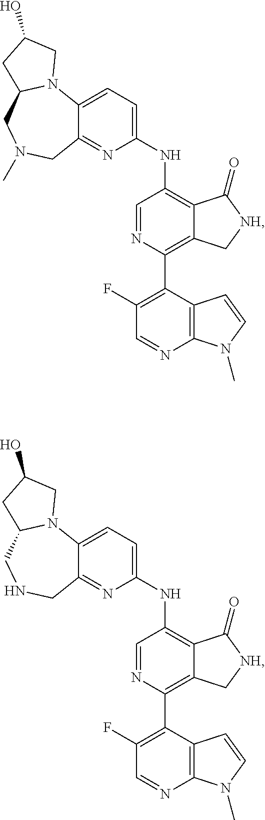 [SMILES: CN1Cc2nc(Nc3cnc(-c4c(F)cnc5c4ccn5C)c4c3C(=O)NC4)ccc2N2C[C@@H](O)C[C@@H]2C1 ; Cn1ccc2c(-c3ncc(Nc4ccc5c(n4)CNC[C@@H]4C[C@@H](O)CN54)c4c3CNC4=O)c(F)cnc21]

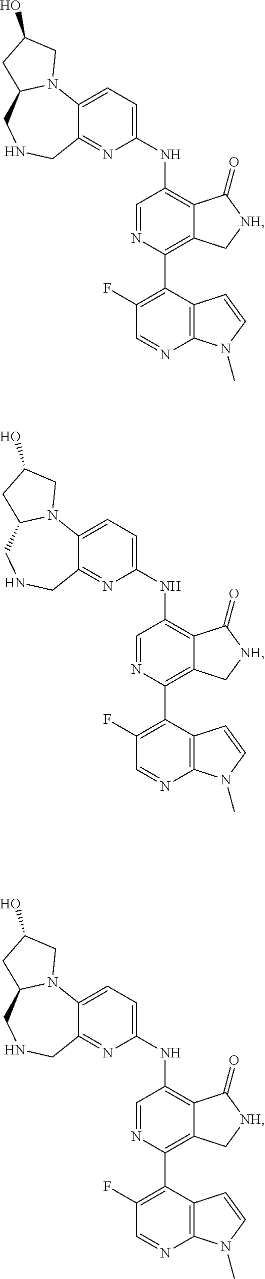 [SMILES: Cn1ccc2c(-c3ncc(Nc4ccc5c(n4)CNC[C@H]4C[C@@H](O)CN54)c4c3CNC4=O)c(F)cnc21 ; Cn1ccc2c(-c3ncc(Nc4ccc5c(n4)CNC[C@@H]4C[C@H](O)CN54)c4c3CNC4=O)c(F)cnc21 ; Cn1ccc2c(-c3ncc(Nc4ccc5c(n4)CNC[C@H]4C[C@H](O)CN54)c4c3CNC4=O)c(F)cnc21]

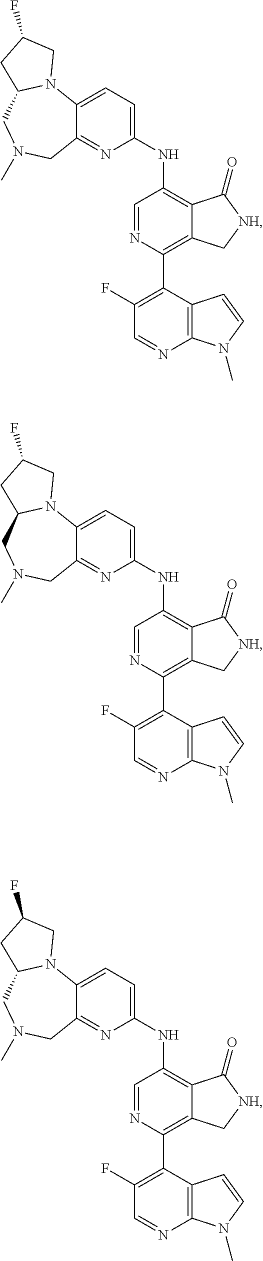 [SMILES: CN1Cc2nc(Nc3cnc(-c4c(F)cnc5c4ccn5C)c4c3C(=O)NC4)ccc2N2C[C@@H](F)C[C@H]2C1 ; CN1Cc2nc(Nc3cnc(-c4c(F)cnc5c4ccn5C)c4c3C(=O)NC4)ccc2N2C[C@@H](F)C[C@@H]2C1 ; CN1Cc2nc(Nc3cnc(-c4c(F)cnc5c4ccn5C)c4c3C(=O)NC4)ccc2N2C[C@H](F)C[C@H]2C1]

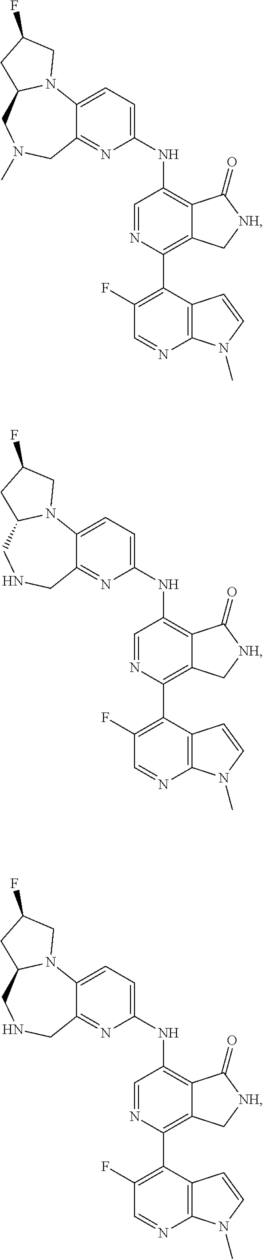 [SMILES: CN1Cc2nc(Nc3cnc(-c4c(F)cnc5c4ccn5C)c4c3C(=O)NC4)ccc2N2C[C@H](F)C[C@@H]2C1 ; Cn1ccc2c(-c3ncc(Nc4ccc5c(n4)CNC[C@@H]4C[C@@H](F)CN54)c4c3CNC4=O)c(F)cnc21 ; Cn1ccc2c(-c3ncc(Nc4ccc5c(n4)CNC[C@H]4C[C@@H](F)CN54)c4c3CNC4=O)c(F)cnc21]

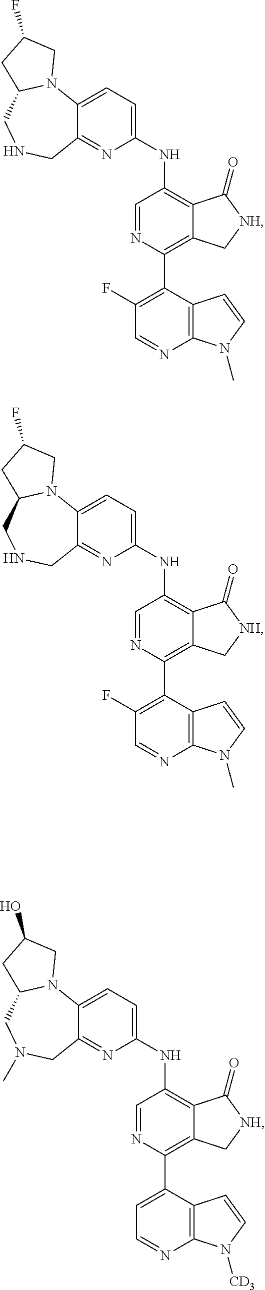 [SMILES: Cn1ccc2c(-c3ncc(Nc4ccc5c(n4)CNC[C@@H]4C[C@H](F)CN54)c4c3CNC4=O)c(F)cnc21 ; Cn1ccc2c(-c3ncc(Nc4ccc5c(n4)CNC[C@H]4C[C@H](F)CN54)c4c3CNC4=O)c(F)cnc21 ; [2H]C([2H])([2H])n1ccc2c(-c3ncc(Nc4ccc5c(n4)CN(C)C[C@@H]4C[C@@H](O)CN54)c4c3CNC4=O)ccnc21]

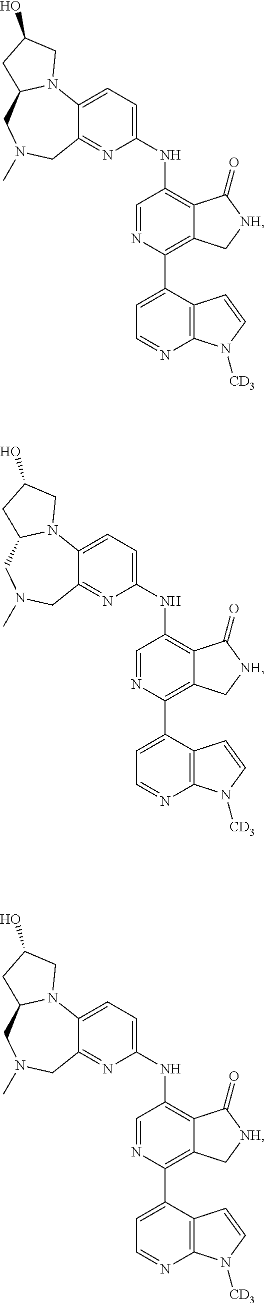 [SMILES: [2H]C([2H])([2H])n1ccc2c(-c3ncc(Nc4ccc5c(n4)CN(C)C[C@H]4C[C@@H](O)CN54)c4c3CNC4=O)ccnc21 ; [2H]C([2H])([2H])n1ccc2c(-c3ncc(Nc4ccc5c(n4)CN(C)C[C@@H]4C[C@H](O)CN54)c4c3CNC4=O)ccnc21 ; [2H]C([2H])([2H])n1ccc2c(-c3ncc(Nc4ccc5c(n4)CN(C)C[C@H]4C[C@H](O)CN54)c4c3CNC4=O)ccnc21]

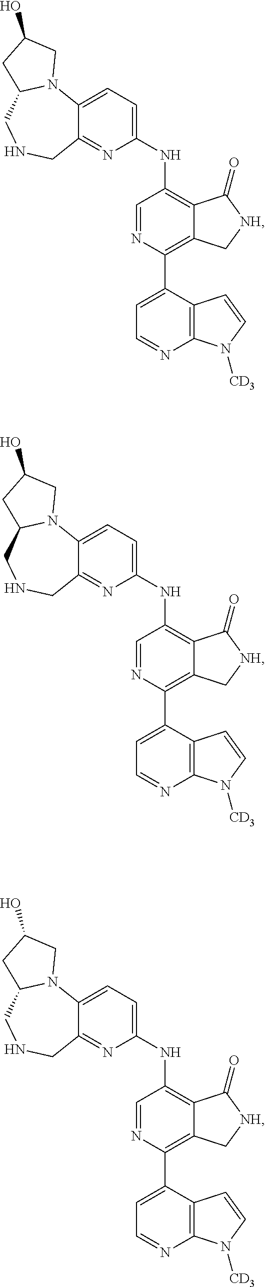 [SMILES: [2H]C([2H])([2H])n1ccc2c(-c3ncc(Nc4ccc5c(n4)CNC[C@@H]4C[C@@H](O)CN54)c4c3CNC4=O)ccnc21 ; [2H]C([2H])([2H])n1ccc2c(-c3ncc(Nc4ccc5c(n4)CNC[C@H]4C[C@@H](O)CN54)c4c3CNC4=O)ccnc21 ; [2H]C([2H])([2H])n1ccc2c(-c3ncc(Nc4ccc5c(n4)CNC[C@@H]4C[C@H](O)CN54)c4c3CNC4=O)ccnc21]

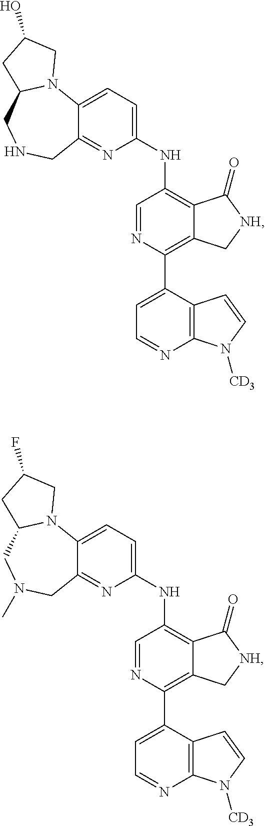 [SMILES: [2H]C([2H])([2H])n1ccc2c(-c3ncc(Nc4ccc5c(n4)CNC[C@H]4C[C@H](O)CN54)c4c3CNC4=O)ccnc21 ; [2H]C([2H])([2H])n1ccc2c(-c3ncc(Nc4ccc5c(n4)CN(C)C[C@@H]4C[C@H](F)CN54)c4c3CNC4=O)ccnc21]

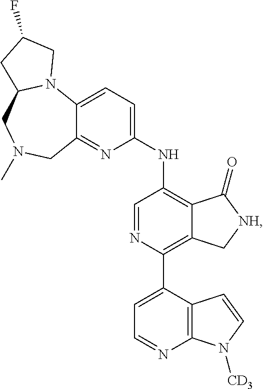 [SMILES: [2H]C([2H])([2H])n1ccc2c(-c3ncc(Nc4ccc5c(n4)CN(C)C[C@H]4C[C@H](F)CN54)c4c3CNC4=O)ccnc21]

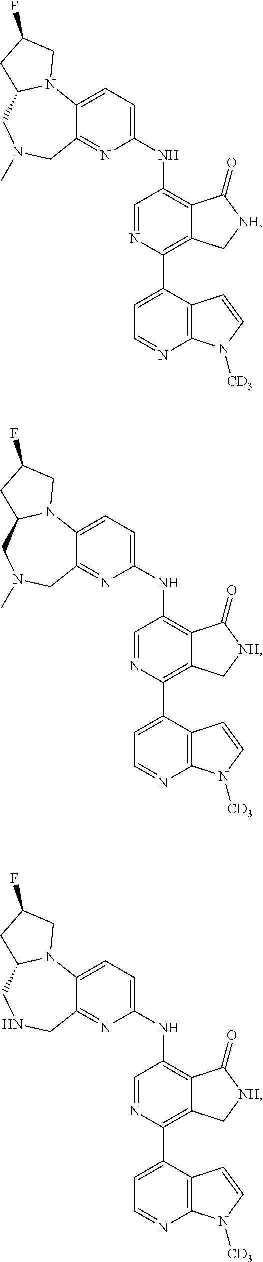 [SMILES: [2H]C([2H])([2H])n1ccc2c(-c3ncc(Nc4ccc5c(n4)CN(C)C[C@@H]4C[C@@H](F)CN54)c4c3CNC4=O)ccnc21 ; [2H]C([2H])([2H])n1ccc2c(-c3ncc(Nc4ccc5c(n4)CN(C)C[C@H]4C[C@@H](F)CN54)c4c3CNC4=O)ccnc21 ; [2H]C([2H])([2H])n1ccc2c(-c3ncc(Nc4ccc5c(n4)CNC[C@@H]4C[C@@H](F)CN54)c4c3CNC4=O)ccnc21]

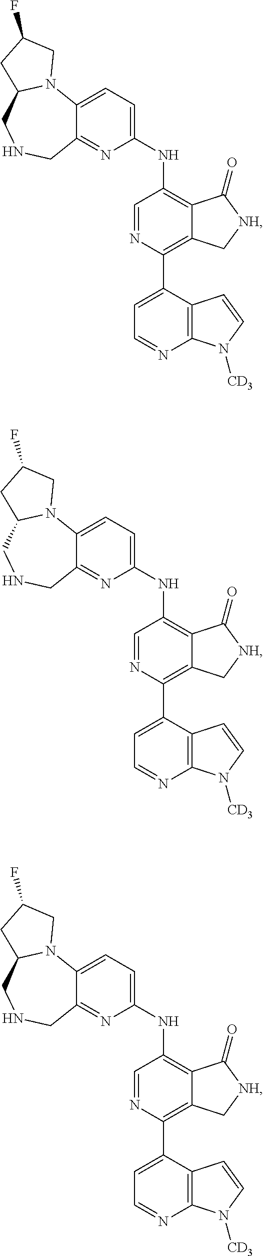 [SMILES: [2H]C([2H])([2H])n1ccc2c(-c3ncc(Nc4ccc5c(n4)CNC[C@H]4C[C@@H](F)CN54)c4c3CNC4=O)ccnc21 ; [2H]C([2H])([2H])n1ccc2c(-c3ncc(Nc4ccc5c(n4)CNC[C@@H]4C[C@H](F)CN54)c4c3CNC4=O)ccnc21 ; [2H]C([2H])([2H])n1ccc2c(-c3ncc(Nc4ccc5c(n4)CNC[C@H]4C[C@H](F)CN54)c4c3CNC4=O)ccnc21]

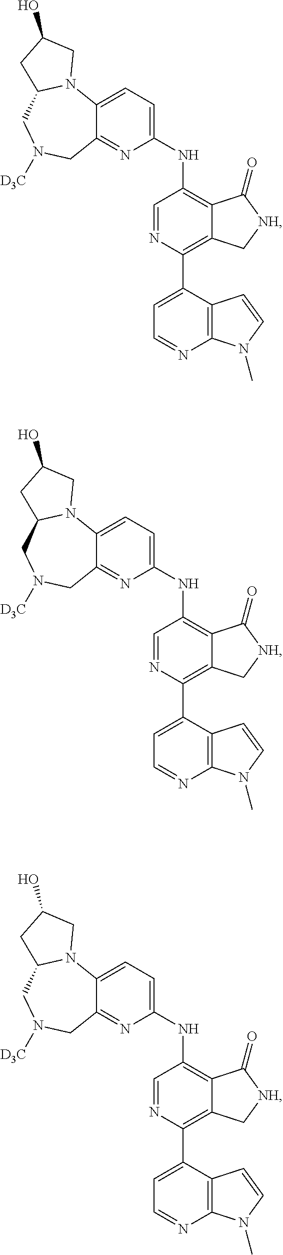 [SMILES: [2H]C([2H])([2H])N1Cc2nc(Nc3cnc(-c4ccnc5c4ccn5C)c4c3C(=O)NC4)ccc2N2C[C@H](O)C[C@H]2C1 ; [2H]C([2H])([2H])N1Cc2nc(Nc3cnc(-c4ccnc5c4ccn5C)c4c3C(=O)NC4)ccc2N2C[C@H](O)C[C@@H]2C1 ; [2H]C([2H])([2H])N1Cc2nc(Nc3cnc(-c4ccnc5c4ccn5C)c4c3C(=O)NC4)ccc2N2C[C@@H](O)C[C@H]2C1]

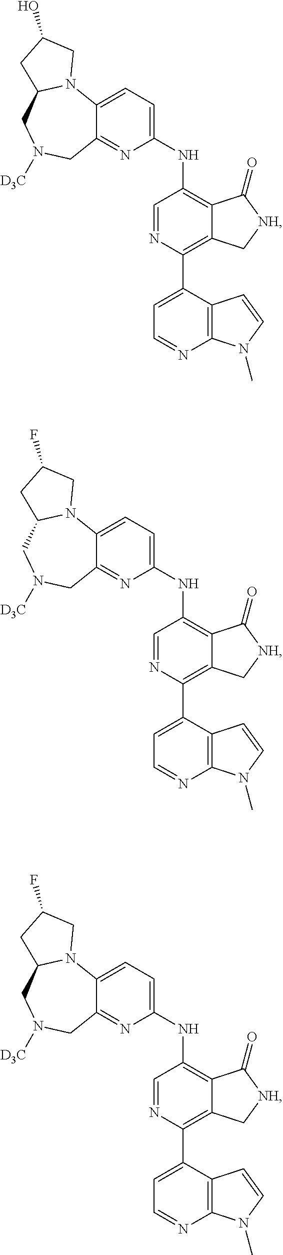 [SMILES: [2H]C([2H])([2H])N1Cc2nc(Nc3cnc(-c4ccnc5c4ccn5C)c4c3C(=O)NC4)ccc2N2C[C@@H](O)C[C@@H]2C1 ; [2H]C([2H])([2H])N1Cc2nc(Nc3cnc(-c4ccnc5c4ccn5C)c4c3C(=O)NC4)ccc2N2C[C@@H](F)C[C@H]2C1 ; [2H]C([2H])([2H])N1Cc2nc(Nc3cnc(-c4ccnc5c4ccn5C)c4c3C(=O)NC4)ccc2N2C[C@@H](F)C[C@@H]2C1]

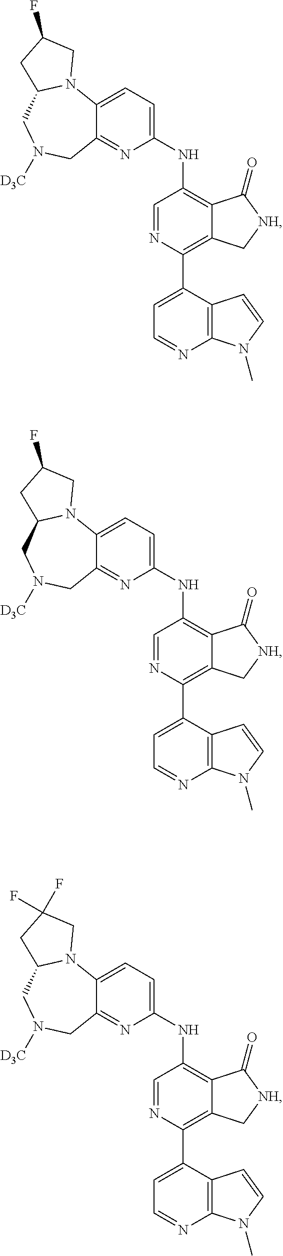 [SMILES: [2H]C([2H])([2H])N1Cc2nc(Nc3cnc(-c4ccnc5c4ccn5C)c4c3C(=O)NC4)ccc2N2C[C@H](F)C[C@H]2C1 ; [2H]C([2H])([2H])N1Cc2nc(Nc3cnc(-c4ccnc5c4ccn5C)c4c3C(=O)NC4)ccc2N2C[C@H](F)C[C@@H]2C1 ; [2H]C([2H])([2H])N1Cc2nc(Nc3cnc(-c4ccnc5c4ccn5C)c4c3C(=O)NC4)ccc2N2CC(F)(F)C[C@H]2C1]

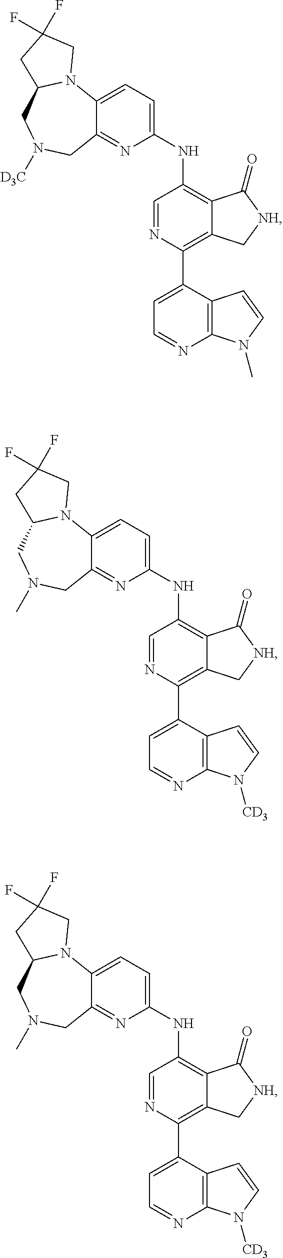 [SMILES: [2H]C([2H])([2H])N1Cc2nc(Nc3cnc(-c4ccnc5c4ccn5C)c4c3C(=O)NC4)ccc2N2CC(F)(F)C[C@@H]2C1 ; [2H]C([2H])([2H])n1ccc2c(-c3ncc(Nc4ccc5c(n4)CN(C)C[C@@H]4CC(F)(F)CN54)c4c3CNC4=O)ccnc21 ; [2H]C([2H])([2H])n1ccc2c(-c3ncc(Nc4ccc5c(n4)CN(C)C[C@H]4CC(F)(F)CN54)c4c3CNC4=O)ccnc21]

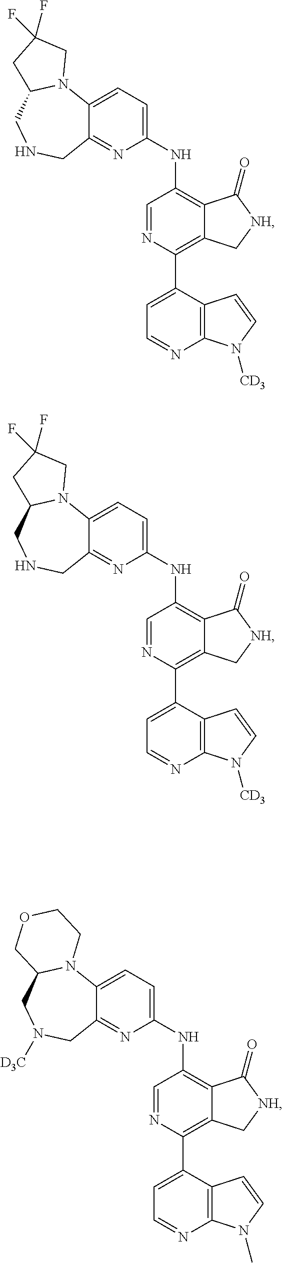 [SMILES: [2H]C([2H])([2H])n1ccc2c(-c3ncc(Nc4ccc5c(n4)CNC[C@@H]4CC(F)(F)CN54)c4c3CNC4=O)ccnc21 ; [2H]C([2H])([2H])n1ccc2c(-c3ncc(Nc4ccc5c(n4)CNC[C@H]4CC(F)(F)CN54)c4c3CNC4=O)ccnc21 ; [2H]C([2H])([2H])N1Cc2nc(Nc3cnc(-c4ccnc5c4ccn5C)c4c3C(=O)NC4)ccc2N2CCOC[C@@H]2C1]

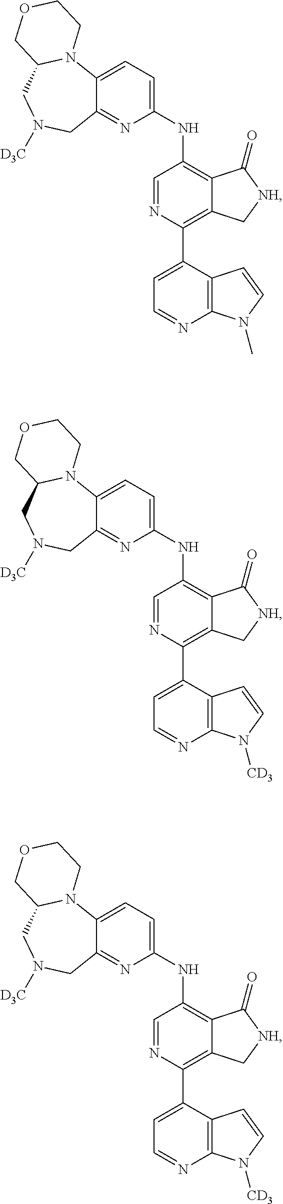 [SMILES: [2H]C([2H])([2H])N1Cc2nc(Nc3cnc(-c4ccnc5c4ccn5C)c4c3C(=O)NC4)ccc2N2CCOC[C@H]2C1 ; [2H]C([2H])([2H])N1Cc2nc(Nc3cnc(-c4ccnc5c4ccn5C([2H])([2H])[2H])c4c3C(=O)NC4)ccc2N2CCOC[C@@H]2C1 ; [2H]C([2H])([2H])N1Cc2nc(Nc3cnc(-c4ccnc5c4ccn5C([2H])([2H])[2H])c4c3C(=O)NC4)ccc2N2CCOC[C@H]2C1]

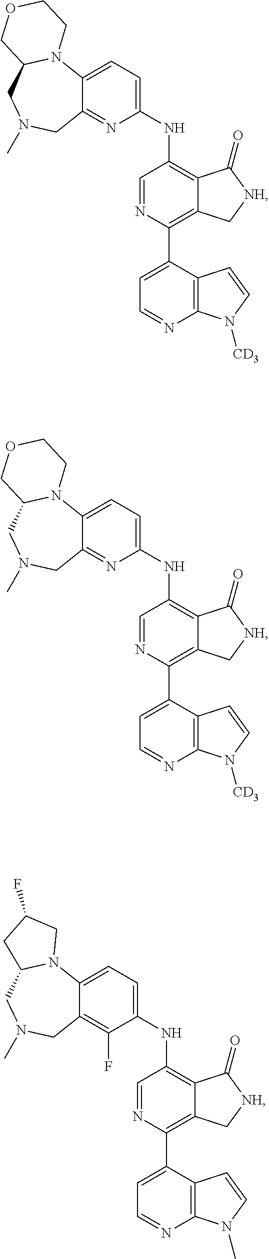 [SMILES: [2H]C([2H])([2H])n1ccc2c(-c3ncc(Nc4ccc5c(n4)CN(C)C[C@H]4COCCN54)c4c3CNC4=O)ccnc21 ; [2H]C([2H])([2H])n1ccc2c(-c3ncc(Nc4ccc5c(n4)CN(C)C[C@@H]4COCCN54)c4c3CNC4=O)ccnc21 ; CN1Cc2c(ccc(Nc3cnc(-c4ccnc5c4ccn5C)c4c3C(=O)NC4)c2F)N2C[C@@H](F)C[C@H]2C1]

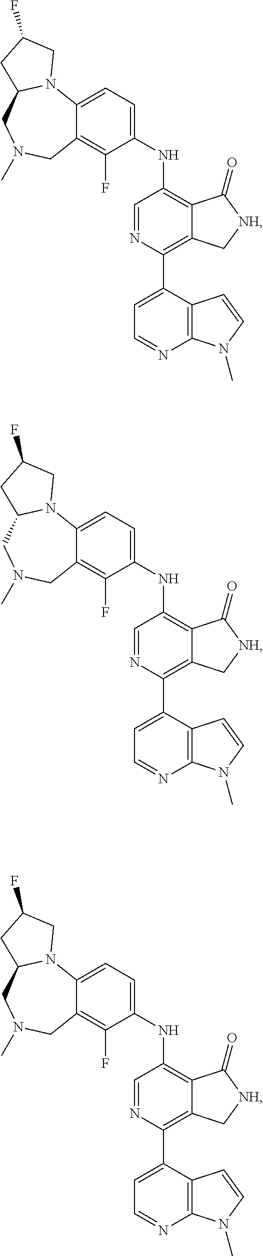 [SMILES: CN1Cc2c(ccc(Nc3cnc(-c4ccnc5c4ccn5C)c4c3C(=O)NC4)c2F)N2C[C@@H](F)C[C@@H]2C1 ; CN1Cc2c(ccc(Nc3cnc(-c4ccnc5c4ccn5C)c4c3C(=O)NC4)c2F)N2C[C@H](F)C[C@H]2C1 ; CN1Cc2c(ccc(Nc3cnc(-c4ccnc5c4ccn5C)c4c3C(=O)NC4)c2F)N2C[C@H](F)C[C@@H]2C1]

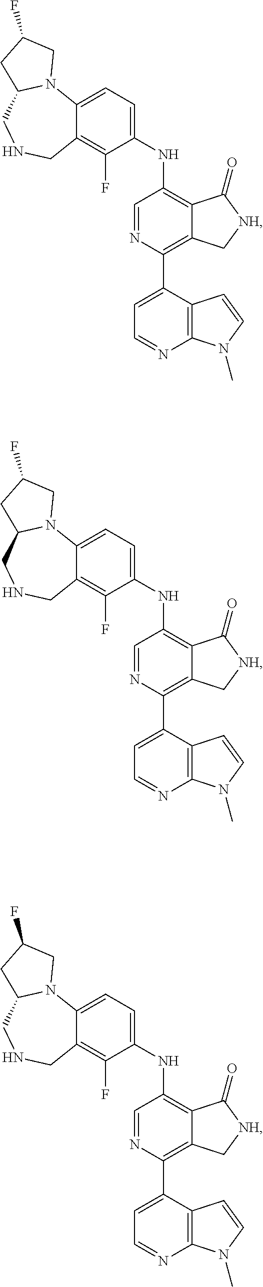 [SMILES: Cn1ccc2c(-c3ncc(Nc4ccc5c(c4F)CNC[C@@H]4C[C@H](F)CN54)c4c3CNC4=O)ccnc21 ; Cn1ccc2c(-c3ncc(Nc4ccc5c(c4F)CNC[C@H]4C[C@H](F)CN54)c4c3CNC4=O)ccnc21 ; Cn1ccc2c(-c3ncc(Nc4ccc5c(c4F)CNC[C@@H]4C[C@@H](F)CN54)c4c3CNC4=O)ccnc21]

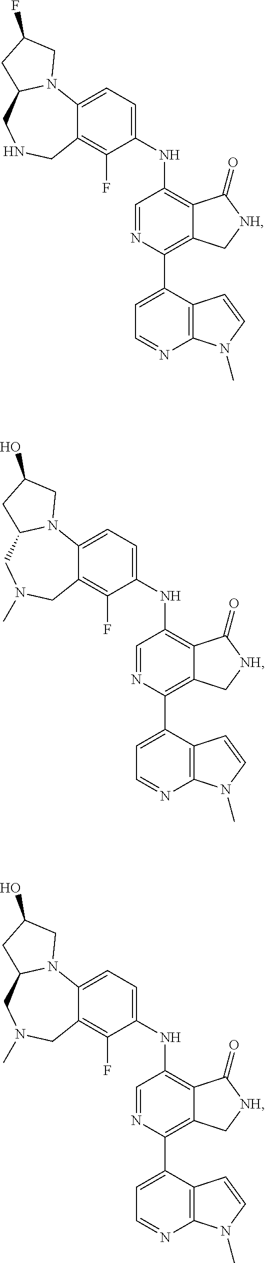 [SMILES: Cn1ccc2c(-c3ncc(Nc4ccc5c(c4F)CNC[C@H]4C[C@@H](F)CN54)c4c3CNC4=O)ccnc21 ; CN1Cc2c(ccc(Nc3cnc(-c4ccnc5c4ccn5C)c4c3C(=O)NC4)c2F)N2C[C@H](O)C[C@H]2C1 ; CN1Cc2c(ccc(Nc3cnc(-c4ccnc5c4ccn5C)c4c3C(=O)NC4)c2F)N2C[C@H](O)C[C@@H]2C1]

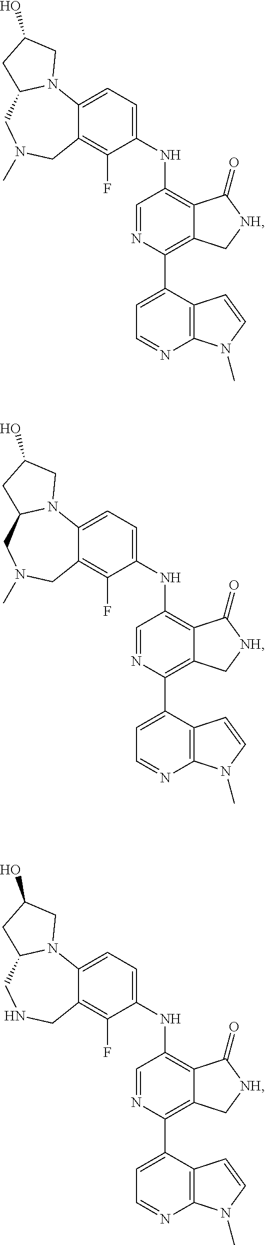 [SMILES: CN1Cc2c(ccc(Nc3cnc(-c4ccnc5c4ccn5C)c4c3C(=O)NC4)c2F)N2C[C@@H](O)C[C@H]2C1 ; CN1Cc2c(ccc(Nc3cnc(-c4ccnc5c4ccn5C)c4c3C(=O)NC4)c2F)N2C[C@@H](O)C[C@@H]2C1 ; Cn1ccc2c(-c3ncc(Nc4ccc5c(c4F)CNC[C@@H]4C[C@@H](O)CN54)c4c3CNC4=O)ccnc21]

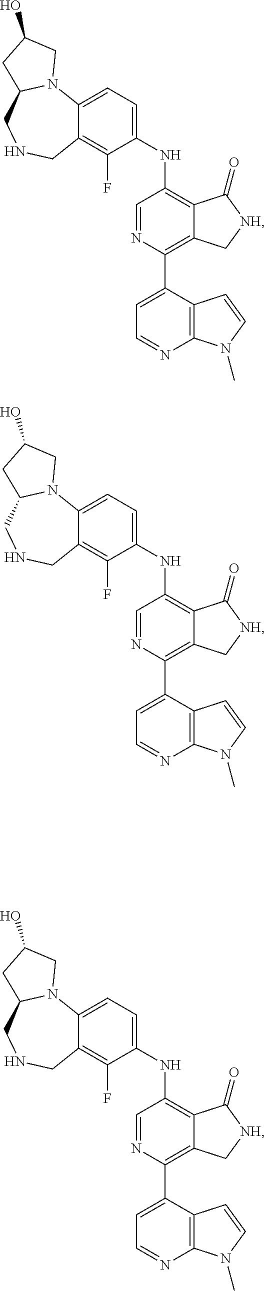 [SMILES: Cn1ccc2c(-c3ncc(Nc4ccc5c(c4F)CNC[C@H]4C[C@@H](O)CN54)c4c3CNC4=O)ccnc21 ; Cn1ccc2c(-c3ncc(Nc4ccc5c(c4F)CNC[C@@H]4C[C@H](O)CN54)c4c3CNC4=O)ccnc21 ; Cn1ccc2c(-c3ncc(Nc4ccc5c(c4F)CNC[C@H]4C[C@H](O)CN54)c4c3CNC4=O)ccnc21]

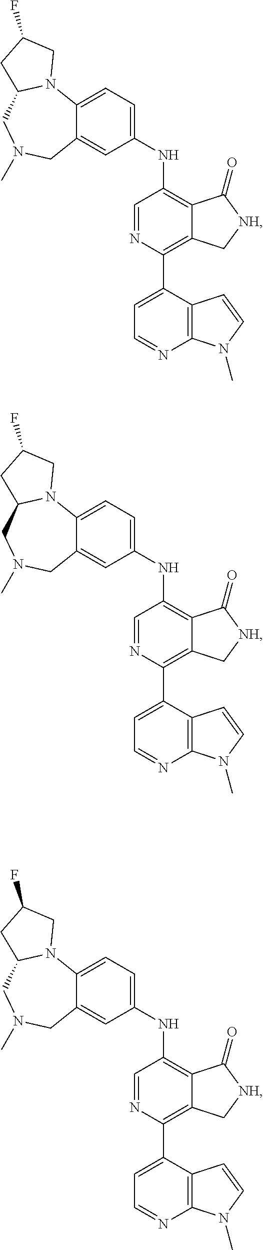 [SMILES: CN1Cc2cc(Nc3cnc(-c4ccnc5c4ccn5C)c4c3C(=O)NC4)ccc2N2C[C@@H](F)C[C@H]2C1 ; CN1Cc2cc(Nc3cnc(-c4ccnc5c4ccn5C)c4c3C(=O)NC4)ccc2N2C[C@@H](F)C[C@@H]2C1 ; CN1Cc2cc(Nc3cnc(-c4ccnc5c4ccn5C)c4c3C(=O)NC4)ccc2N2C[C@H](F)C[C@H]2C1]

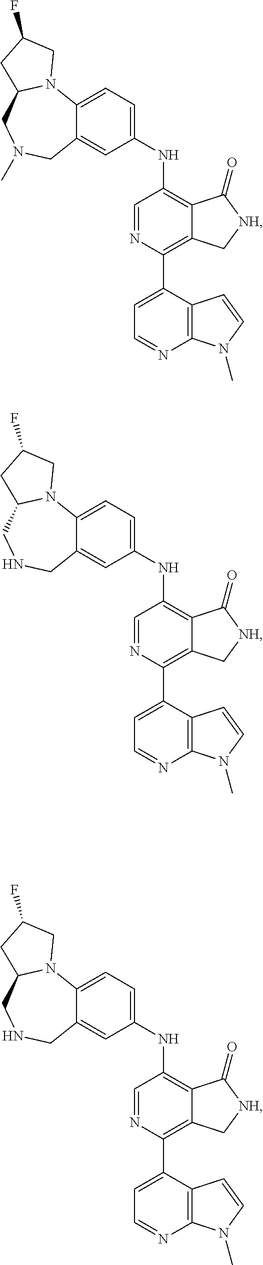 [SMILES: CN1Cc2cc(Nc3cnc(-c4ccnc5c4ccn5C)c4c3C(=O)NC4)ccc2N2C[C@H](F)C[C@@H]2C1 ; Cn1ccc2c(-c3ncc(Nc4ccc5c(c4)CNC[C@@H]4C[C@H](F)CN54)c4c3CNC4=O)ccnc21 ; Cn1ccc2c(-c3ncc(Nc4ccc5c(c4)CNC[C@H]4C[C@H](F)CN54)c4c3CNC4=O)ccnc21]

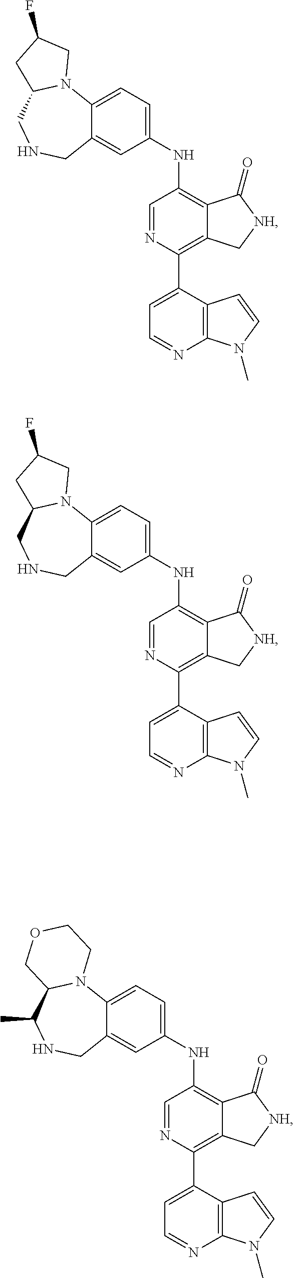 [SMILES: Cn1ccc2c(-c3ncc(Nc4ccc5c(c4)CNC[C@@H]4C[C@@H](F)CN54)c4c3CNC4=O)ccnc21 ; Cn1ccc2c(-c3ncc(Nc4ccc5c(c4)CNC[C@H]4C[C@@H](F)CN54)c4c3CNC4=O)ccnc21 ; C[C@@H]1NCc2cc(Nc3cnc(-c4ccnc5c4ccn5C)c4c3C(=O)NC4)ccc2N2CCOC[C@H]12]

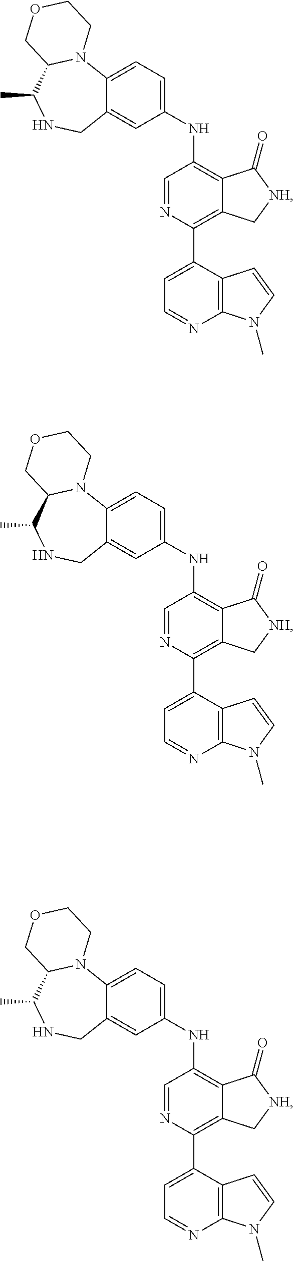 [SMILES: C[C@@H]1NCc2cc(Nc3cnc(-c4ccnc5c4ccn5C)c4c3C(=O)NC4)ccc2N2CCOC[C@@H]12 ; C[C@H]1NCc2cc(Nc3cnc(-c4ccnc5c4ccn5C)c4c3C(=O)NC4)ccc2N2CCOC[C@H]12 ; C[C@H]1NCc2cc(Nc3cnc(-c4ccnc5c4ccn5C)c4c3C(=O)NC4)ccc2N2CCOC[C@@H]12]

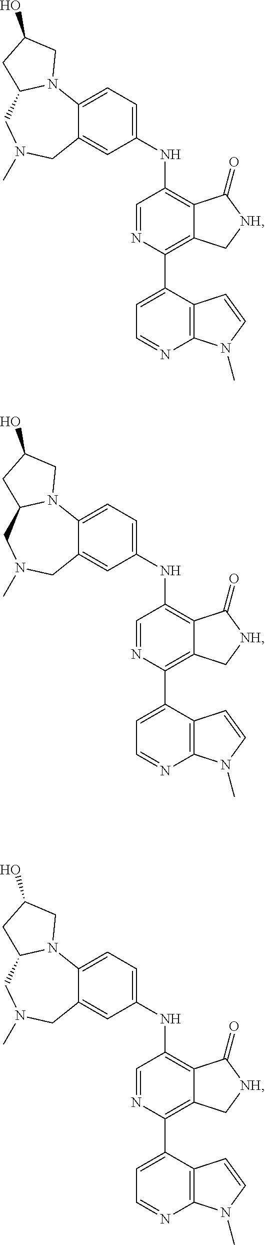 [SMILES: CN1Cc2cc(Nc3cnc(-c4ccnc5c4ccn5C)c4c3C(=O)NC4)ccc2N2C[C@H](O)C[C@H]2C1 ; CN1Cc2cc(Nc3cnc(-c4ccnc5c4ccn5C)c4c3C(=O)NC4)ccc2N2C[C@H](O)C[C@@H]2C1 ; CN1Cc2cc(Nc3cnc(-c4ccnc5c4ccn5C)c4c3C(=O)NC4)ccc2N2C[C@@H](O)C[C@H]2C1]

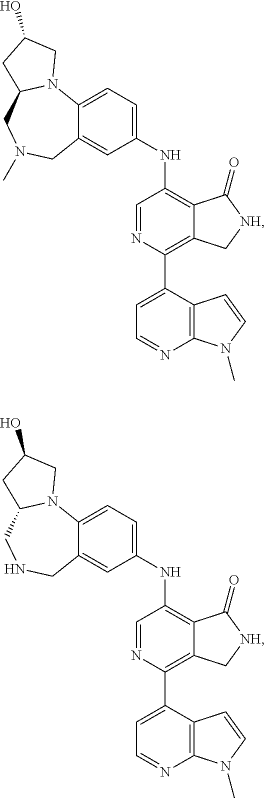 [SMILES: CN1Cc2cc(Nc3cnc(-c4ccnc5c4ccn5C)c4c3C(=O)NC4)ccc2N2C[C@@H](O)C[C@@H]2C1 ; Cn1ccc2c(-c3ncc(Nc4ccc5c(c4)CNC[C@@H]4C[C@@H](O)CN54)c4c3CNC4=O)ccnc21]

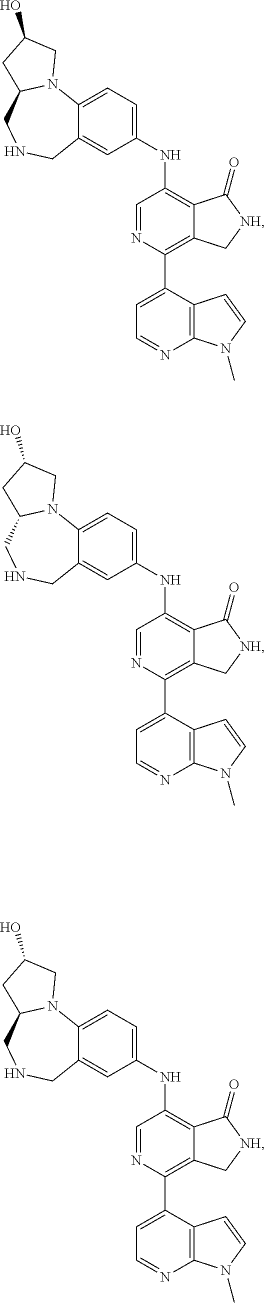 [SMILES: Cn1ccc2c(-c3ncc(Nc4ccc5c(c4)CNC[C@H]4C[C@@H](O)CN54)c4c3CNC4=O)ccnc21 ; Cn1ccc2c(-c3ncc(Nc4ccc5c(c4)CNC[C@@H]4C[C@H](O)CN54)c4c3CNC4=O)ccnc21 ; Cn1ccc2c(-c3ncc(Nc4ccc5c(c4)CNC[C@H]4C[C@H](O)CN54)c4c3CNC4=O)ccnc21]

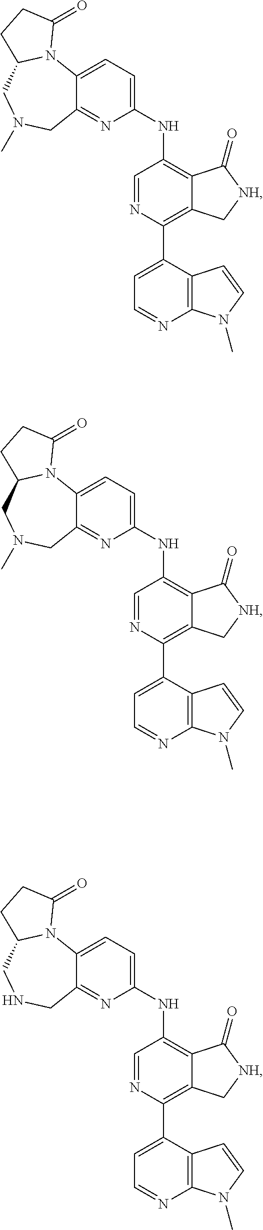 [SMILES: CN1Cc2nc(Nc3cnc(-c4ccnc5c4ccn5C)c4c3C(=O)NC4)ccc2N2C(=O)CC[C@H]2C1 ; CN1Cc2nc(Nc3cnc(-c4ccnc5c4ccn5C)c4c3C(=O)NC4)ccc2N2C(=O)CC[C@@H]2C1 ; Cn1ccc2c(-c3ncc(Nc4ccc5c(n4)CNC[C@@H]4CCC(=O)N54)c4c3CNC4=O)ccnc21]

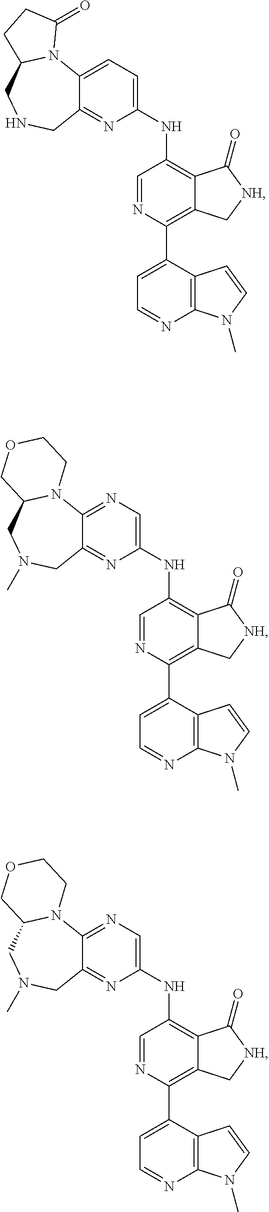 [SMILES: Cn1ccc2c(-c3ncc(Nc4ccc5c(n4)CNC[C@H]4CCC(=O)N54)c4c3CNC4=O)ccnc21 ; CN1Cc2nc(Nc3cnc(-c4ccnc5c4ccn5C)c4c3C(=O)NC4)cnc2N2CCOC[C@@H]2C1 ; CN1Cc2nc(Nc3cnc(-c4ccnc5c4ccn5C)c4c3C(=O)NC4)cnc2N2CCOC[C@H]2C1]

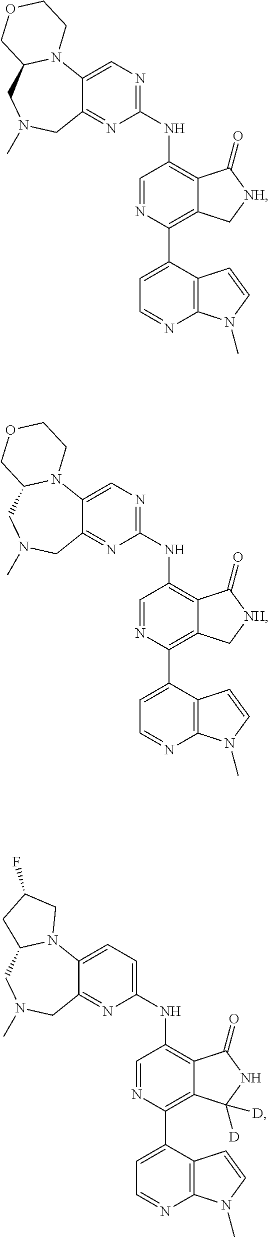

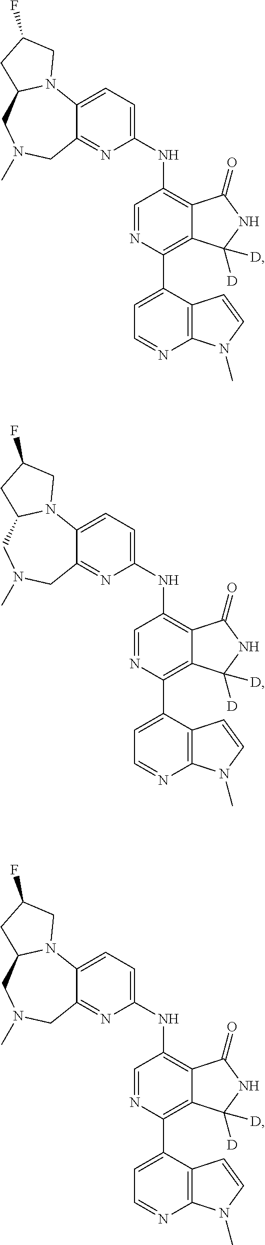

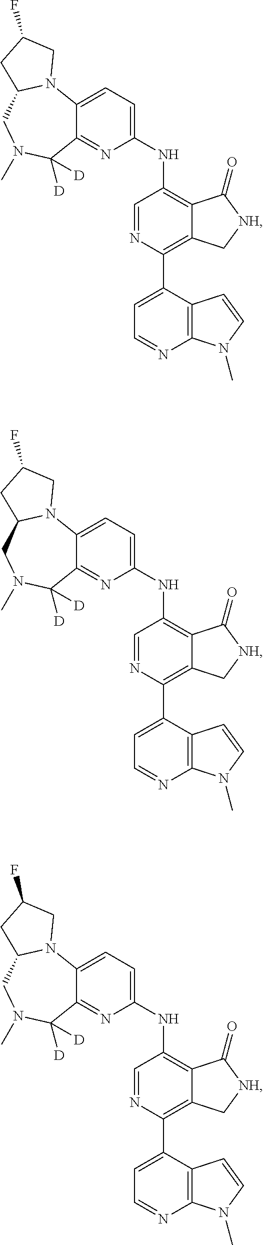 [SMILES: [2H]C1([2H])c2nc(Nc3cnc(-c4ccnc5c4ccn5C)c4c3C(=O)NC4)ccc2N2C[C@@H](F)C[C@H]2CN1C ; [2H]C1([2H])c2nc(Nc3cnc(-c4ccnc5c4ccn5C)c4c3C(=O)NC4)ccc2N2C[C@@H](F)C[C@@H]2CN1C ; [2H]C1([2H])c2nc(Nc3cnc(-c4ccnc5c4ccn5C)c4c3C(=O)NC4)ccc2N2C[C@H](F)C[C@H]2CN1C]

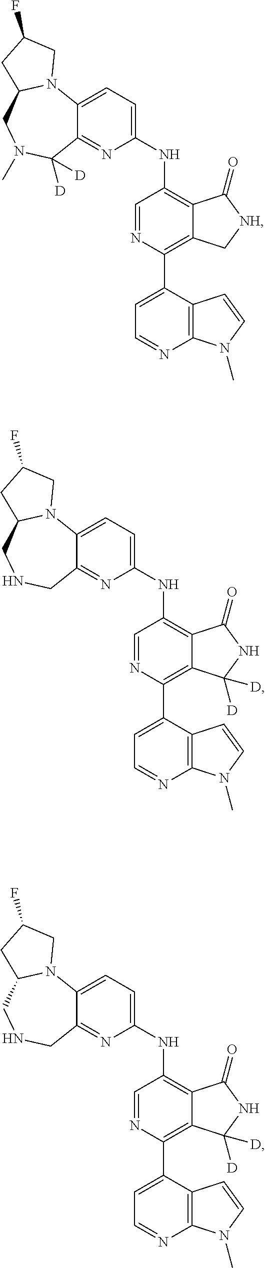

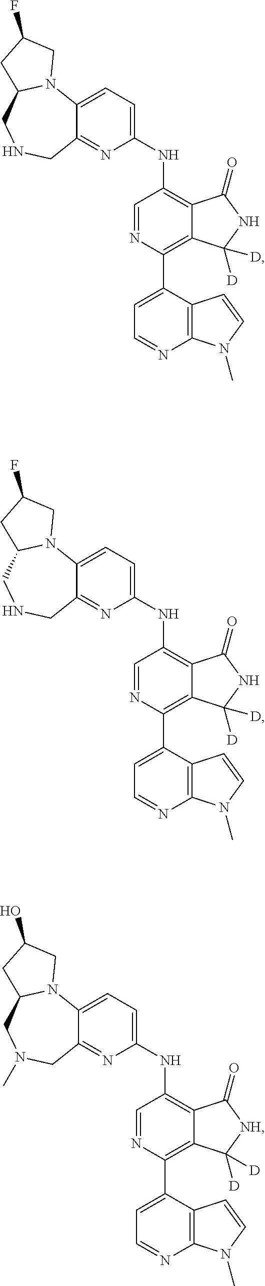

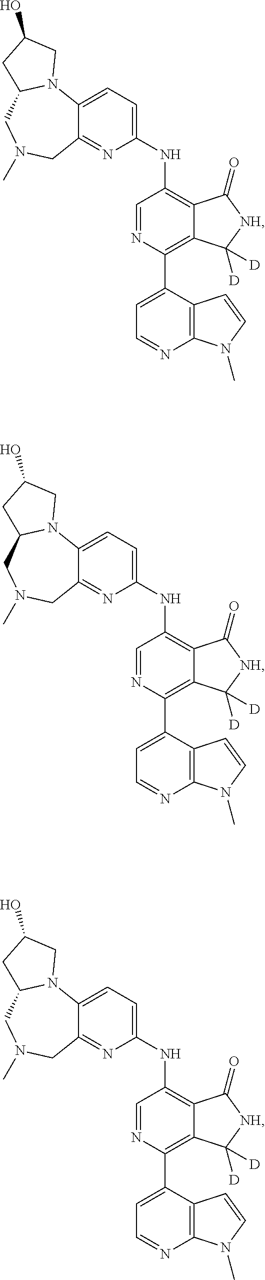 [SMILES: [2H]C1([2H])NC(=O)c2c(Nc3ccc4c(n3)CN(C)C[C@@H]3C[C@@H](O)CN43)cnc(-c3ccnc4c3ccn4C)c21 ; [2H]C1([2H])NC(=O)c2c(Nc3ccc4c(n3)CN(C)C[C@H]3C[C@H](O)CN43)cnc(-c3ccnc4c3ccn4C)c21 ; [2H]C1([2H])NC(=O)c2c(Nc3ccc4c(n3)CN(C)C[C@@H]3C[C@H](O)CN43)cnc(-c3ccnc4c3ccn4C)c21]

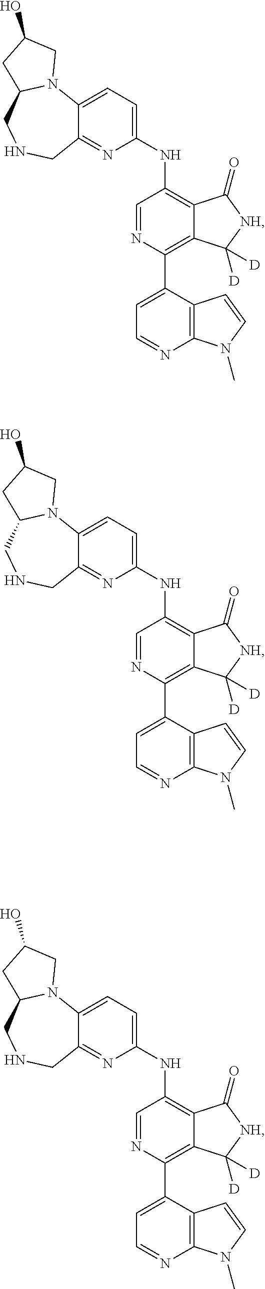 [SMILES: [2H]C1([2H])NC(=O)c2c(Nc3ccc4c(n3)CNC[C@H]3C[C@@H](O)CN43)cnc(-c3ccnc4c3ccn4C)c21 ; [2H]C1([2H])NC(=O)c2c(Nc3ccc4c(n3)CNC[C@@H]3C[C@@H](O)CN43)cnc(-c3ccnc4c3ccn4C)c21 ; [2H]C1([2H])NC(=O)c2c(Nc3ccc4c(n3)CNC[C@H]3C[C@H](O)CN43)cnc(-c3ccnc4c3ccn4C)c21]

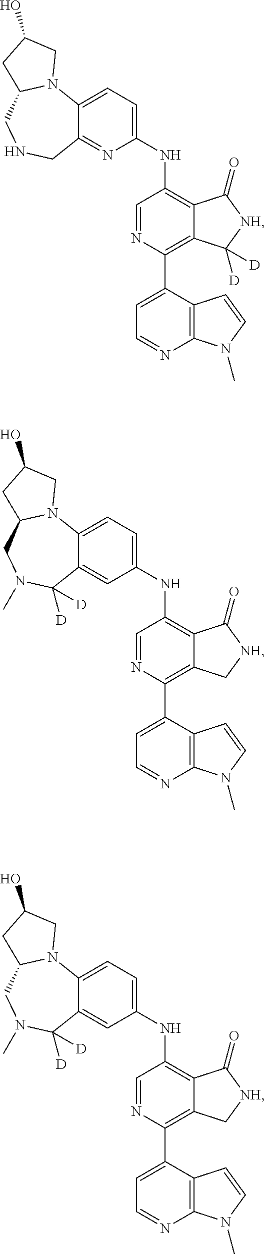 [SMILES: [2H]C1([2H])NC(=O)c2c(Nc3ccc4c(n3)CNC[C@@H]3C[C@H](O)CN43)cnc(-c3ccnc4c3ccn4C)c21 ; [2H]C1([2H])c2cc(Nc3cnc(-c4ccnc5c4ccn5C)c4c3C(=O)NC4)ccc2N2C[C@H](O)C[C@@H]2CN1C ; [2H]C1([2H])c2cc(Nc3cnc(-c4ccnc5c4ccn5C)c4c3C(=O)NC4)ccc2N2C[C@H](O)C[C@H]2CN1C]

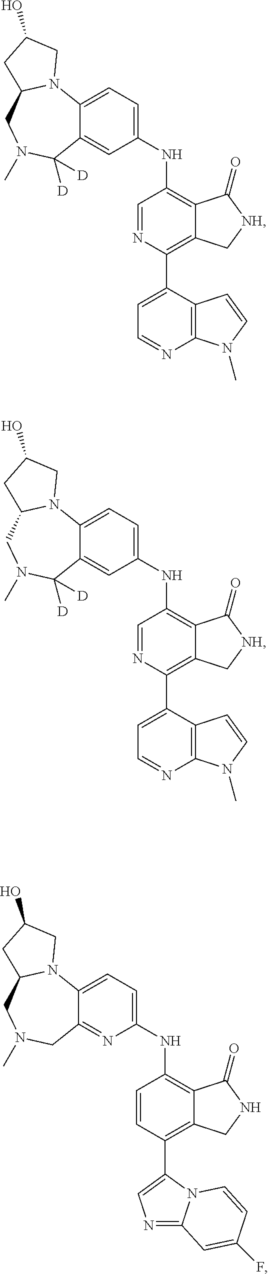 [SMILES: [2H]C1([2H])c2cc(Nc3cnc(-c4ccnc5c4ccn5C)c4c3C(=O)NC4)ccc2N2C[C@@H](O)C[C@@H]2CN1C ; [2H]C1([2H])c2cc(Nc3cnc(-c4ccnc5c4ccn5C)c4c3C(=O)NC4)ccc2N2C[C@@H](O)C[C@H]2CN1C ; CN1Cc2nc(Nc3ccc(-c4cnc5cc(F)ccn45)c4c3C(=O)NC4)ccc2N2C[C@H](O)C[C@@H]2C1]

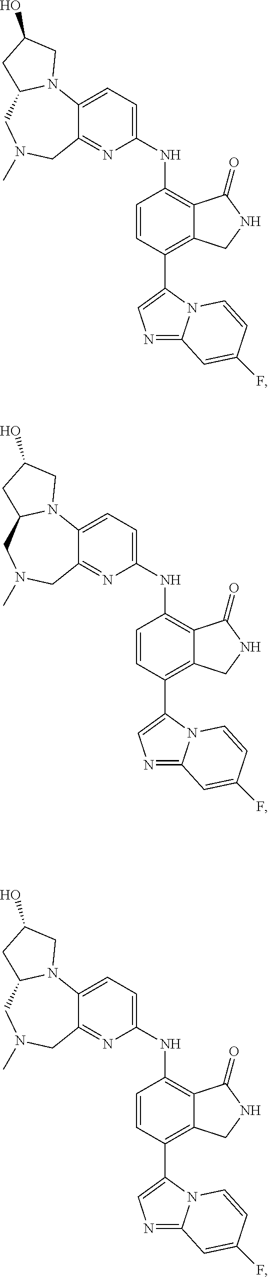 [SMILES: CN1Cc2nc(Nc3ccc(-c4cnc5cc(F)ccn45)c4c3C(=O)NC4)ccc2N2C[C@H](O)C[C@H]2C1 ; CN1Cc2nc(Nc3ccc(-c4cnc5cc(F)ccn45)c4c3C(=O)NC4)ccc2N2C[C@@H](O)C[C@@H]2C1 ; CN1Cc2nc(Nc3ccc(-c4cnc5cc(F)ccn45)c4c3C(=O)NC4)ccc2N2C[C@@H](O)C[C@H]2C1]

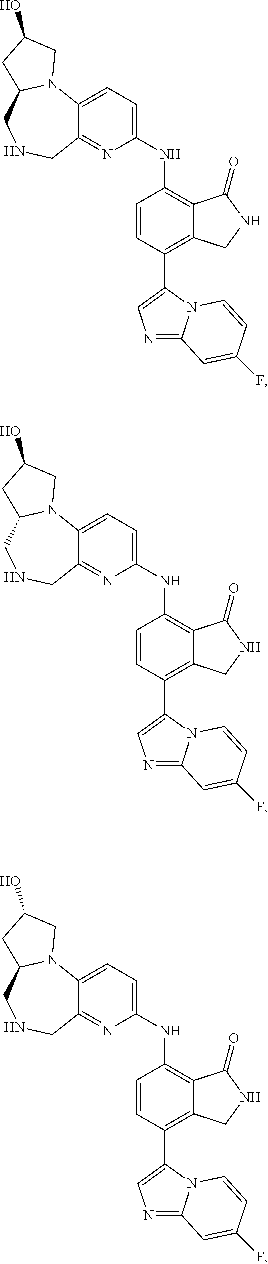 [SMILES: O=C1NCc2c(-c3cnc4cc(F)ccn34)ccc(Nc3ccc4c(n3)CNC[C@H]3C[C@@H](O)CN43)c21 ; O=C1NCc2c(-c3cnc4cc(F)ccn34)ccc(Nc3ccc4c(n3)CNC[C@@H]3C[C@@H](O)CN43)c21 ; O=C1NCc2c(-c3cnc4cc(F)ccn34)ccc(Nc3ccc4c(n3)CNC[C@H]3C[C@H](O)CN43)c21]

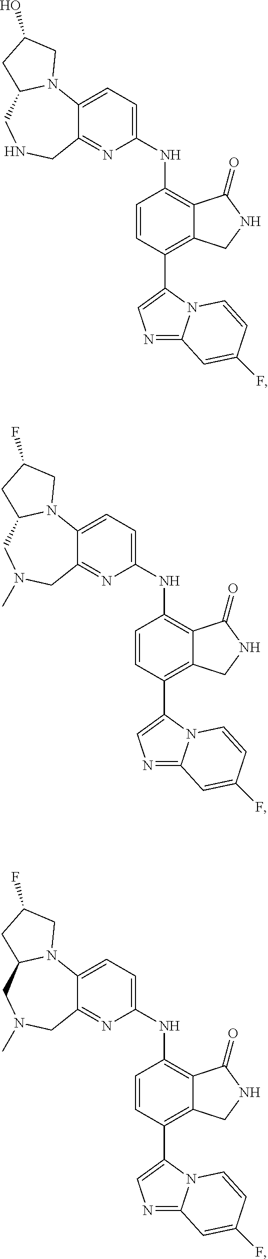 [SMILES: O=C1NCc2c(-c3cnc4cc(F)ccn34)ccc(Nc3ccc4c(n3)CNC[C@@H]3C[C@H](O)CN43)c21 ; CN1Cc2nc(Nc3ccc(-c4cnc5cc(F)ccn45)c4c3C(=O)NC4)ccc2N2C[C@@H](F)C[C@H]2C1 ; CN1Cc2nc(Nc3ccc(-c4cnc5cc(F)ccn45)c4c3C(=O)NC4)ccc2N2C[C@@H](F)C[C@@H]2C1]

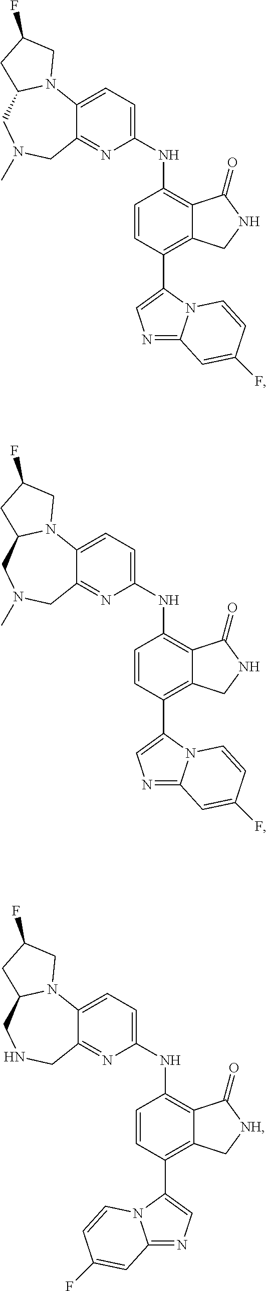 [SMILES: CN1Cc2nc(Nc3ccc(-c4cnc5cc(F)ccn45)c4c3C(=O)NC4)ccc2N2C[C@H](F)C[C@H]2C1 ; CN1Cc2nc(Nc3ccc(-c4cnc5cc(F)ccn45)c4c3C(=O)NC4)ccc2N2C[C@H](F)C[C@@H]2C1 ; O=C1NCc2c(-c3cnc4cc(F)ccn34)ccc(Nc3ccc4c(n3)CNC[C@H]3C[C@@H](F)CN43)c21]

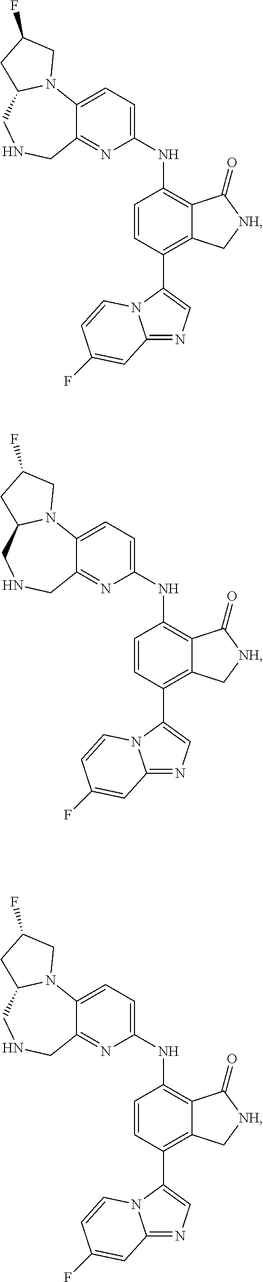 [SMILES: O=C1NCc2c(-c3cnc4cc(F)ccn34)ccc(Nc3ccc4c(n3)CNC[C@@H]3C[C@@H](F)CN43)c21 ; O=C1NCc2c(-c3cnc4cc(F)ccn34)ccc(Nc3ccc4c(n3)CNC[C@H]3C[C@H](F)CN43)c21 ; O=C1NCc2c(-c3cnc4cc(F)ccn34)ccc(Nc3ccc4c(n3)CNC[C@@H]3C[C@H](F)CN43)c21]

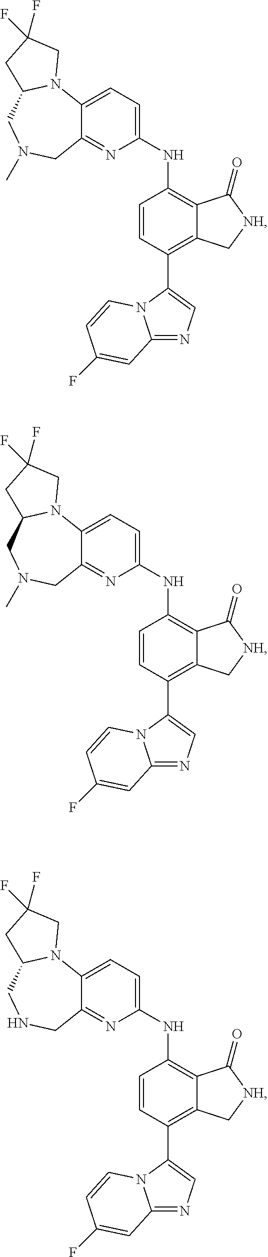 [SMILES: CN1Cc2nc(Nc3ccc(-c4cnc5cc(F)ccn45)c4c3C(=O)NC4)ccc2N2CC(F)(F)C[C@H]2C1 ; CN1Cc2nc(Nc3ccc(-c4cnc5cc(F)ccn45)c4c3C(=O)NC4)ccc2N2CC(F)(F)C[C@@H]2C1 ; O=C1NCc2c(-c3cnc4cc(F)ccn34)ccc(Nc3ccc4c(n3)CNC[C@@H]3CC(F)(F)CN43)c21]

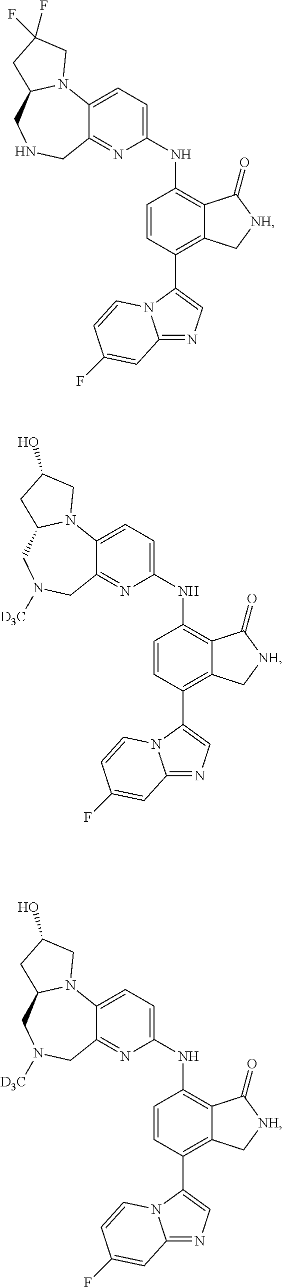 [SMILES: O=C1NCc2c(-c3cnc4cc(F)ccn34)ccc(Nc3ccc4c(n3)CNC[C@H]3CC(F)(F)CN43)c21 ; [2H]C([2H])([2H])N1Cc2nc(Nc3ccc(-c4cnc5cc(F)ccn45)c4c3C(=O)NC4)ccc2N2C[C@@H](O)C[C@H]2C1 ; [2H]C([2H])([2H])N1Cc2nc(Nc3ccc(-c4cnc5cc(F)ccn45)c4c3C(=O)NC4)ccc2N2C[C@@H](O)C[C@@H]2C1]

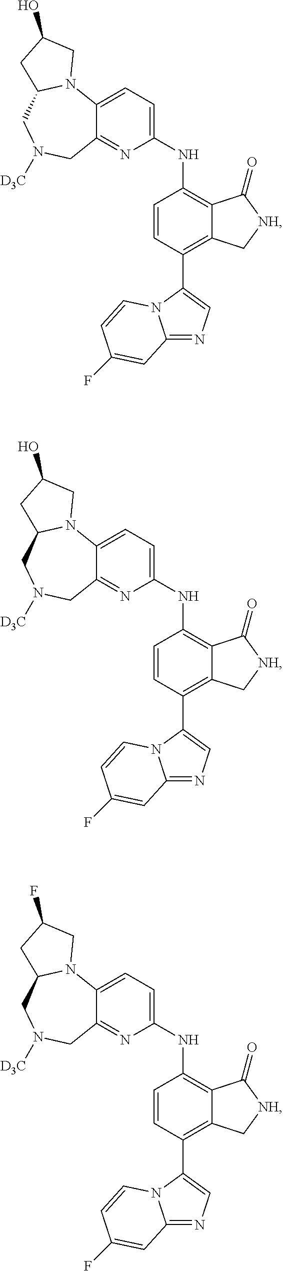 [SMILES: [2H]C([2H])([2H])N1Cc2nc(Nc3ccc(-c4cnc5cc(F)ccn45)c4c3C(=O)NC4)ccc2N2C[C@H](O)C[C@H]2C1 ; [2H]C([2H])([2H])N1Cc2nc(Nc3ccc(-c4cnc5cc(F)ccn45)c4c3C(=O)NC4)ccc2N2C[C@H](O)C[C@@H]2C1 ; [2H]C([2H])([2H])N1Cc2nc(Nc3ccc(-c4cnc5cc(F)ccn45)c4c3C(=O)NC4)ccc2N2C[C@H](F)C[C@@H]2C1]

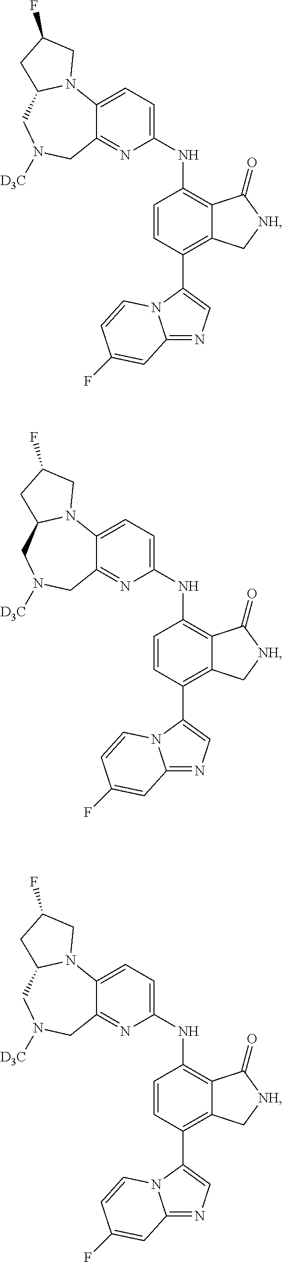 [SMILES: [2H]C([2H])([2H])N1Cc2nc(Nc3ccc(-c4cnc5cc(F)ccn45)c4c3C(=O)NC4)ccc2N2C[C@H](F)C[C@H]2C1 ; [2H]C([2H])([2H])N1Cc2nc(Nc3ccc(-c4cnc5cc(F)ccn45)c4c3C(=O)NC4)ccc2N2C[C@@H](F)C[C@@H]2C1 ; [2H]C([2H])([2H])N1Cc2nc(Nc3ccc(-c4cnc5cc(F)ccn45)c4c3C(=O)NC4)ccc2N2C[C@@H](F)C[C@H]2C1]

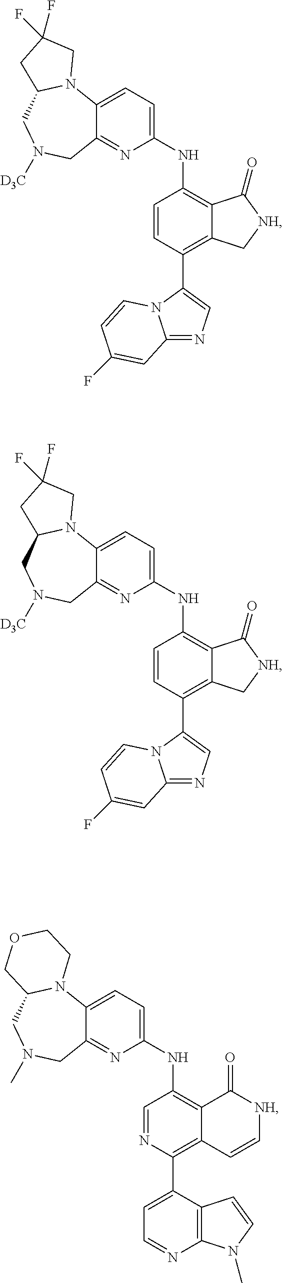 [SMILES: [2H]C([2H])([2H])N1Cc2nc(Nc3ccc(-c4cnc5cc(F)ccn45)c4c3C(=O)NC4)ccc2N2CC(F)(F)C[C@H]2C1 ; [2H]C([2H])([2H])N1Cc2nc(Nc3ccc(-c4cnc5cc(F)ccn45)c4c3C(=O)NC4)ccc2N2CC(F)(F)C[C@@H]2C1 ; CN1Cc2nc(Nc3cnc(-c4ccnc5c4ccn5C)c4cc[nH]c(=O)c34)ccc2N2CCOC[C@H]2C1]

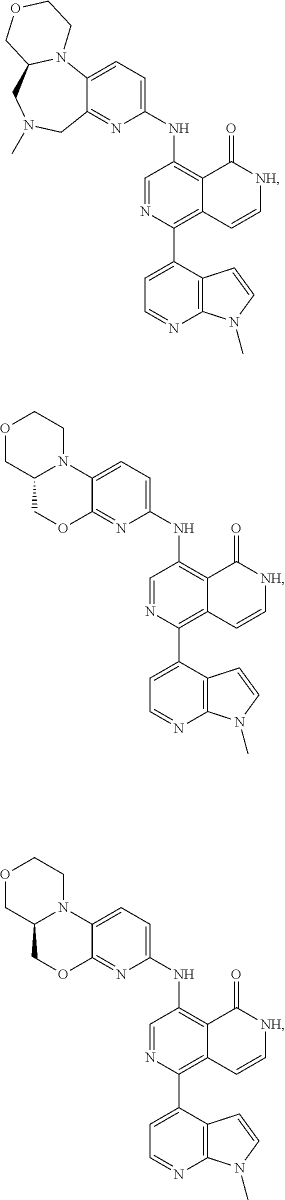 [SMILES: CN1Cc2nc(Nc3cnc(-c4ccnc5c4ccn5C)c4cc[nH]c(=O)c34)ccc2N2CCOC[C@@H]2C1 ; Cn1ccc2c(-c3ncc(Nc4ccc5c(n4)OC[C@@H]4COCCN54)c4c(=O)[nH]ccc34)ccnc21 ; Cn1ccc2c(-c3ncc(Nc4ccc5c(n4)OC[C@H]4COCCN54)c4c(=O)[nH]ccc34)ccnc21]

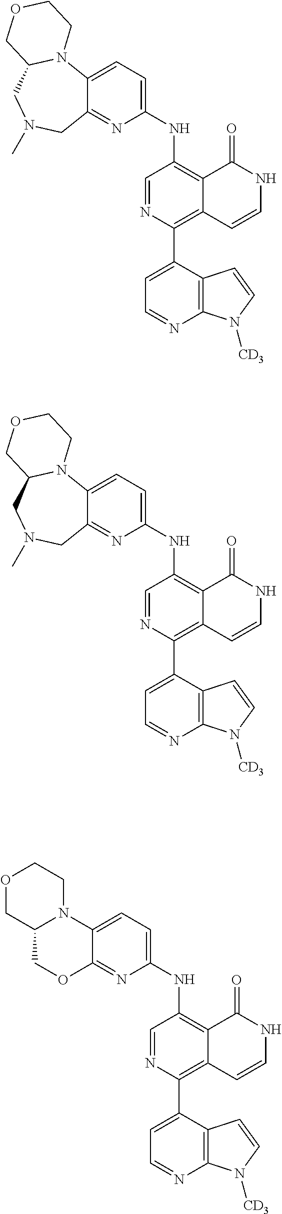 [SMILES: [2H]C([2H])([2H])n1ccc2c(-c3ncc(Nc4ccc5c(n4)CN(C)C[C@@H]4COCCN54)c4c(=O)[nH]ccc34)ccnc21 ; [2H]C([2H])([2H])n1ccc2c(-c3ncc(Nc4ccc5c(n4)CN(C)C[C@H]4COCCN54)c4c(=O)[nH]ccc34)ccnc21 ; [2H]C([2H])([2H])n1ccc2c(-c3ncc(Nc4ccc5c(n4)OC[C@@H]4COCCN54)c4c(=O)[nH]ccc34)ccnc21]

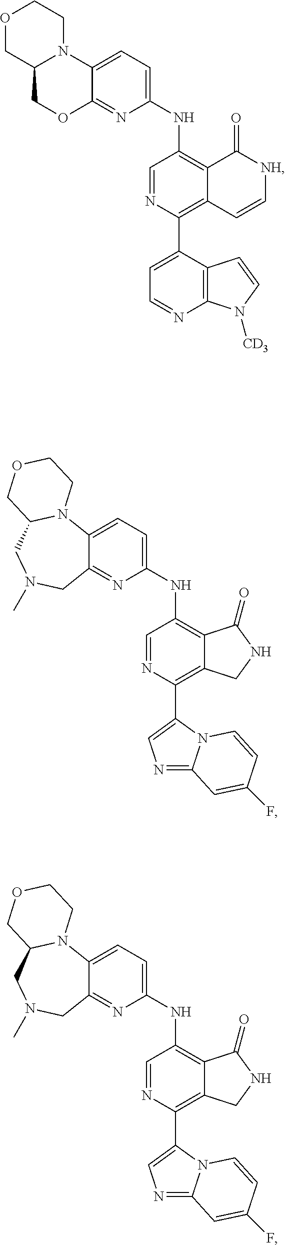 [SMILES: [2H]C([2H])([2H])n1ccc2c(-c3ncc(Nc4ccc5c(n4)OC[C@H]4COCCN54)c4c(=O)[nH]ccc34)ccnc21 ; CN1Cc2nc(Nc3cnc(-c4cnc5cc(F)ccn45)c4c3C(=O)NC4)ccc2N2CCOC[C@H]2C1 ; CN1Cc2nc(Nc3cnc(-c4cnc5cc(F)ccn45)c4c3C(=O)NC4)ccc2N2CCOC[C@@H]2C1]

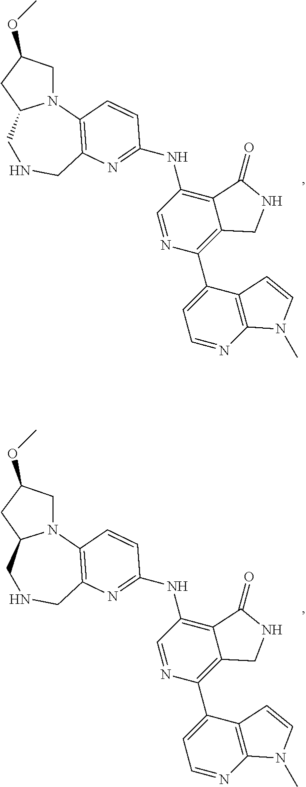 [SMILES: CO[C@@H]1C[C@H]2CNCc3nc(Nc4cnc(-c5ccnc6c5ccn6C)c5c4C(=O)NC5)ccc3N2C1 ; CO[C@@H]1C[C@@H]2CNCc3nc(Nc4cnc(-c5ccnc6c5ccn6C)c5c4C(=O)NC5)ccc3N2C1]

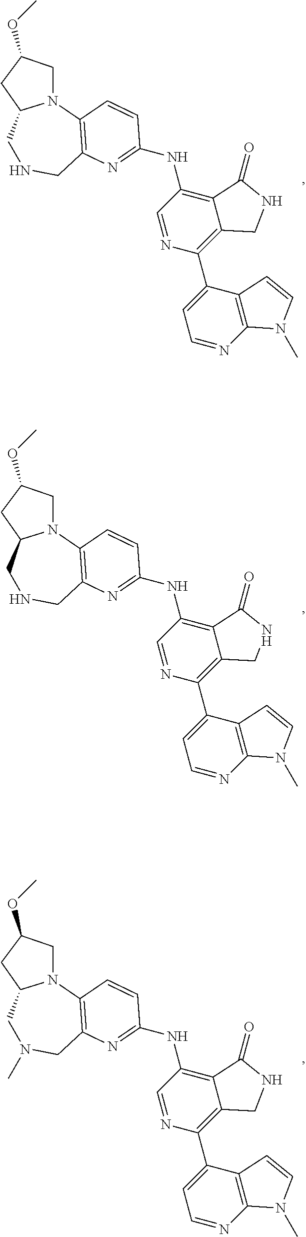 [SMILES: CO[C@H]1C[C@H]2CNCc3nc(Nc4cnc(-c5ccnc6c5ccn6C)c5c4C(=O)NC5)ccc3N2C1 ; CO[C@H]1C[C@@H]2CNCc3nc(Nc4cnc(-c5ccnc6c5ccn6C)c5c4C(=O)NC5)ccc3N2C1 ; CO[C@@H]1C[C@H]2CN(C)Cc3nc(Nc4cnc(-c5ccnc6c5ccn6C)c5c4C(=O)NC5)ccc3N2C1]

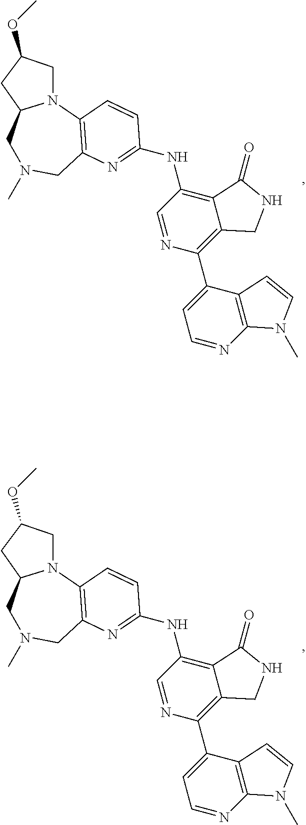 [SMILES: CO[C@@H]1C[C@@H]2CN(C)Cc3nc(Nc4cnc(-c5ccnc6c5ccn6C)c5c4C(=O)NC5)ccc3N2C1 ; CO[C@H]1C[C@@H]2CN(C)Cc3nc(Nc4cnc(-c5ccnc6c5ccn6C)c5c4C(=O)NC5)ccc3N2C1]

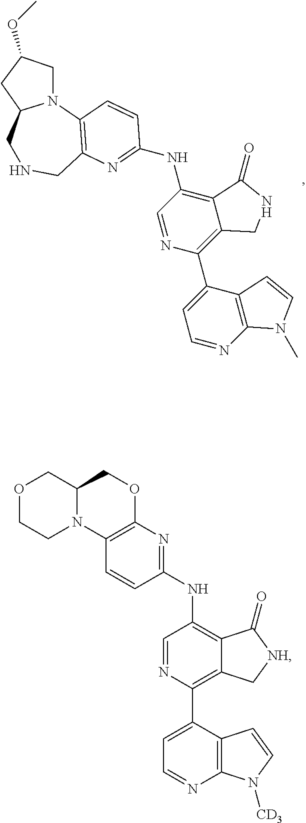 [SMILES: CO[C@H]1C[C@@H]2CNCc3nc(Nc4cnc(-c5ccnc6c5ccn6C)c5c4C(=O)NC5)ccc3N2C1 ; [2H]C([2H])([2H])n1ccc2c(-c3ncc(Nc4ccc5c(n4)OC[C@@H]4COCCN54)c4c3CNC4=O)ccnc21]

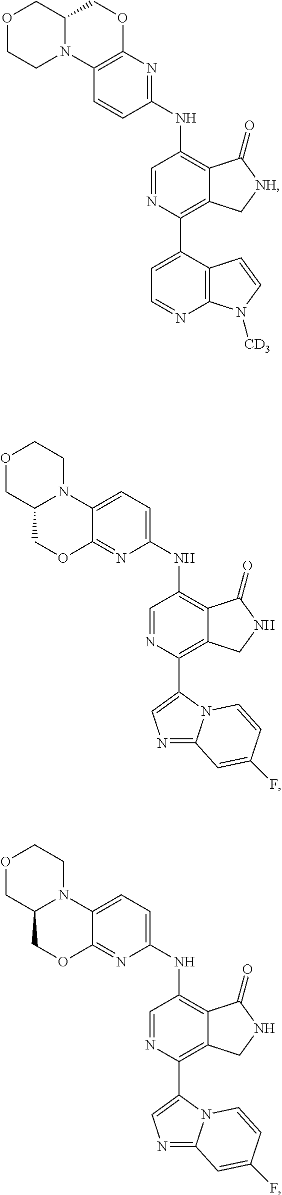 [SMILES: [2H]C([2H])([2H])n1ccc2c(-c3ncc(Nc4ccc5c(n4)OC[C@H]4COCCN54)c4c3CNC4=O)ccnc21 ; O=C1NCc2c(-c3cnc4cc(F)ccn34)ncc(Nc3ccc4c(n3)OC[C@@H]3COCCN43)c21 ; O=C1NCc2c(-c3cnc4cc(F)ccn34)ncc(Nc3ccc4c(n3)OC[C@H]3COCCN43)c21]

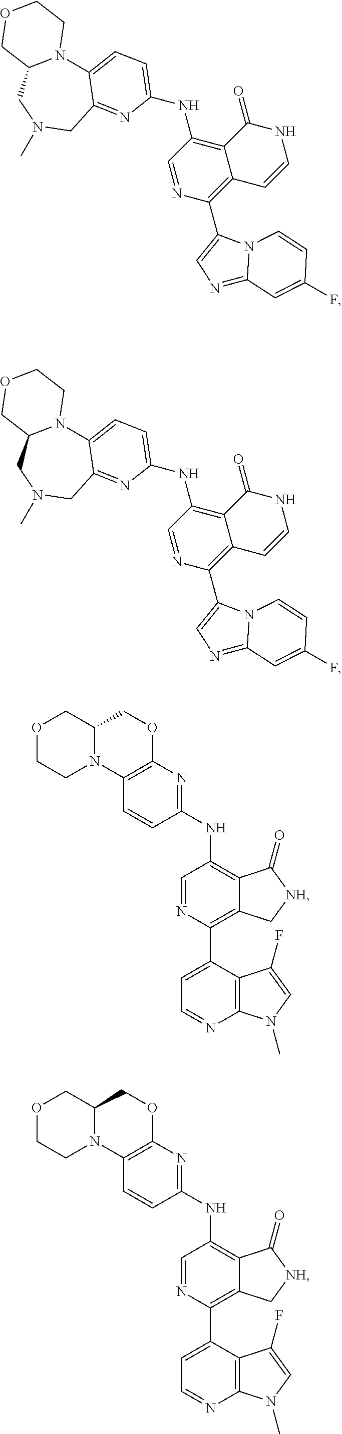 [SMILES: CN1Cc2nc(Nc3cnc(-c4cnc5cc(F)ccn45)c4cc[nH]c(=O)c34)ccc2N2CCOC[C@H]2C1 ; CN1Cc2nc(Nc3cnc(-c4cnc5cc(F)ccn45)c4cc[nH]c(=O)c34)ccc2N2CCOC[C@@H]2C1 ; Cn1cc(F)c2c(-c3ncc(Nc4ccc5c(n4)OC[C@H]4COCCN54)c4c3CNC4=O)ccnc21 ; Cn1cc(F)c2c(-c3ncc(Nc4ccc5c(n4)OC[C@@H]4COCCN54)c4c3CNC4=O)ccnc21]

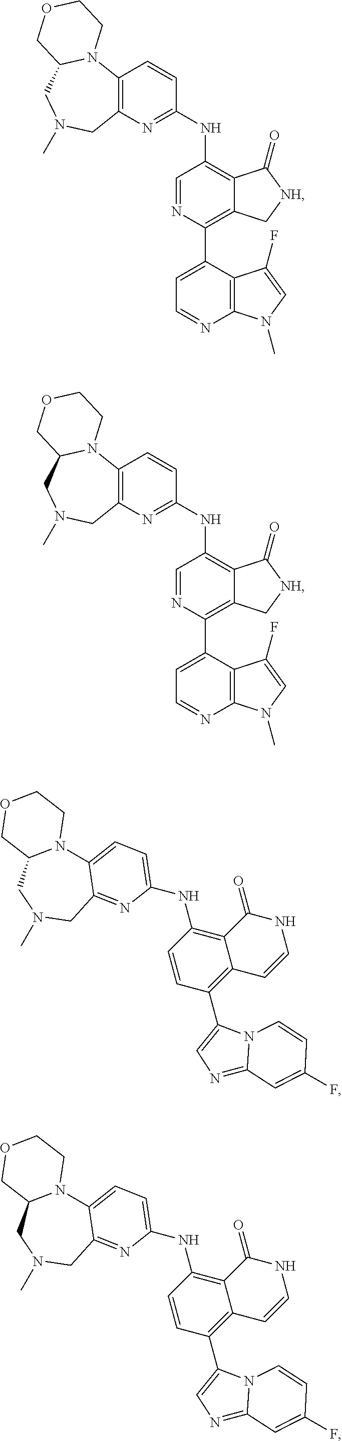 [SMILES: CN1Cc2nc(Nc3cnc(-c4ccnc5c4c(F)cn5C)c4c3C(=O)NC4)ccc2N2CCOC[C@H]2C1 ; CN1Cc2nc(Nc3cnc(-c4ccnc5c4c(F)cn5C)c4c3C(=O)NC4)ccc2N2CCOC[C@@H]2C1 ; CN1Cc2nc(Nc3ccc(-c4cnc5cc(F)ccn45)c4cc[nH]c(=O)c34)ccc2N2CCOC[C@H]2C1 ; CN1Cc2nc(Nc3ccc(-c4cnc5cc(F)ccn45)c4cc[nH]c(=O)c34)ccc2N2CCOC[C@@H]2C1]

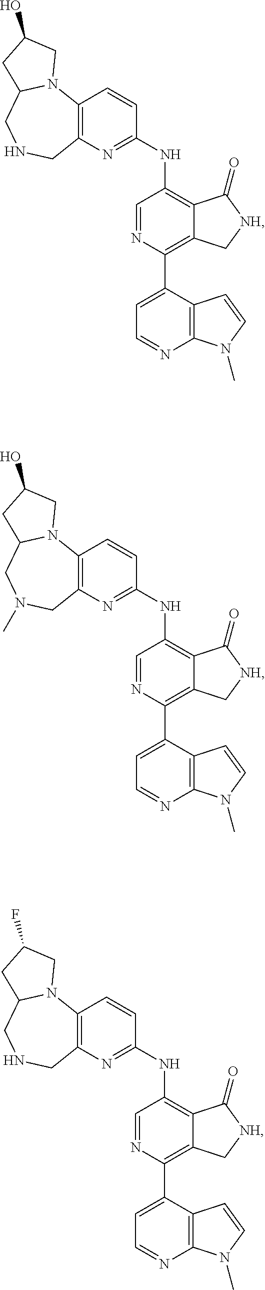 [SMILES: Cn1ccc2c(-c3ncc(Nc4ccc5c(n4)CNCC4C[C@@H](O)CN54)c4c3CNC4=O)ccnc21 ; CN1Cc2nc(Nc3cnc(-c4ccnc5c4ccn5C)c4c3C(=O)NC4)ccc2N2C[C@H](O)CC2C1 ; Cn1ccc2c(-c3ncc(Nc4ccc5c(n4)CNCC4C[C@H](F)CN54)c4c3CNC4=O)ccnc21]

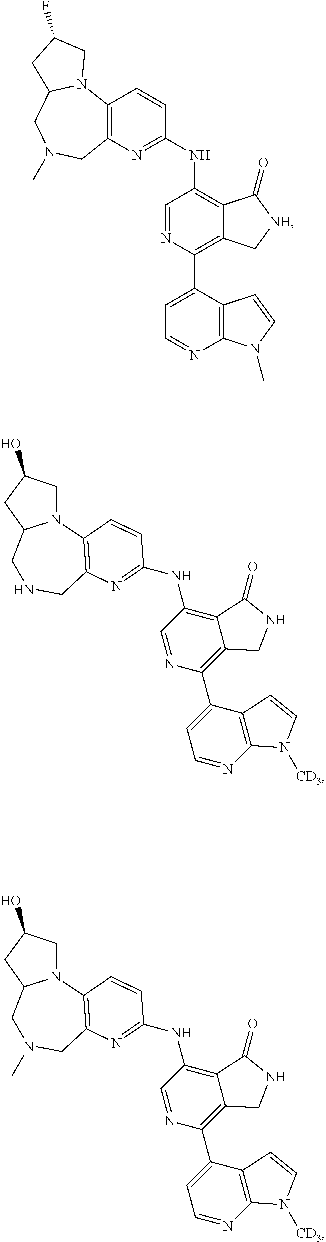 [SMILES: CN1Cc2nc(Nc3cnc(-c4ccnc5c4ccn5C)c4c3C(=O)NC4)ccc2N2C[C@@H](F)CC2C1 ; [2H]C([2H])([2H])n1ccc2c(-c3ncc(Nc4ccc5c(n4)CNCC4C[C@@H](O)CN54)c4c3CNC4=O)ccnc21 ; [2H]C([2H])([2H])n1ccc2c(-c3ncc(Nc4ccc5c(n4)CN(C)CC4C[C@@H](O)CN54)c4c3CNC4=O)ccnc21]

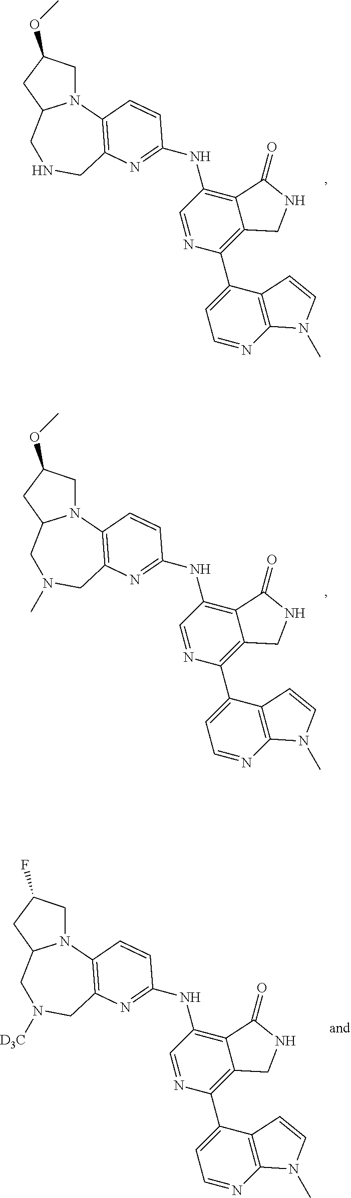 [SMILES: CO[C@@H]1CC2CNCc3nc(Nc4cnc(-c5ccnc6c5ccn6C)c5c4C(=O)NC5)ccc3N2C1 ; CO[C@@H]1CC2CN(C)Cc3nc(Nc4cnc(-c5ccnc6c5ccn6C)c5c4C(=O)NC5)ccc3N2C1 ; [2H]C([2H])([2H])N1Cc2nc(Nc3cnc(-c4ccnc5c4ccn5C)c4c3C(=O)NC4)ccc2N2C[C@@H](F)CC2C1]

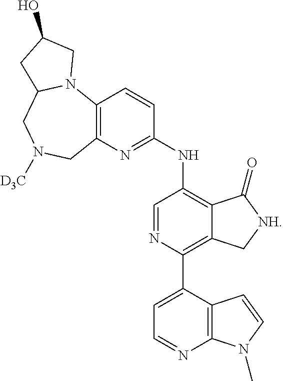 [SMILES: [2H]C([2H])([2H])N1Cc2nc(Nc3cnc(-c4ccnc5c4ccn5C)c4c3C(=O)NC4)ccc2N2C[C@H](O)CC2C1]

18. A method of treating various types of tumors in a subject in need thereof, comprising administering to the subject the compound according to claim 1, a stereoisomer or a pharmaceutically acceptable salt thereof.

19. A method of treating various types of tumors in a subject in need thereof, comprising administering to the subject the compound according to claim 16, a stereoisomer or a pharmaceutically acceptable salt thereof.

20. A method of treating various types of tumors in a subject in need thereof, comprising administering to the subject the compound according to claim 17, a stereoisomer or a pharmaceutically acceptable salt thereof.
